(19) United States
(12) Patent Application Publication
(10) Pub. No.: US 2026/0049143 A1
(43) Pub. Date: Feb. 19, 2026

(54) MULTISPECIFIC ANTIBODY WITH COMBINATION THERAPY FOR IMMUNO-ONCOLOGY

(71) Applicant: KYMAB LIMITED, Cambridge (GB)

(72) Inventors: Jamie Iain CAMPBELL, Cambridge (GB); Nikole SANDY, Cambridge (GB); Cassandra VAN KRINKS, Cambridge (GB); Stephen John ARKINSTALL, Cambridge (GB); Volker GERMASCHEWSKI, Cambridge (GB); Ian KIRBY, Cambridge (GB); Miha KOSMAC, Cambridge (GB); Thomas GALLAGHER, Cambridge (GB); Cecilia DEANTONIO, Cambridge (GB); Stephen Douglas GILLIES, Cambridge (GB); Matthew John MCCOURT, Cambridge (GB); Richard Charles Alfred SAINSON, Cambridge (GB); Mohammed Hanif ALI, Cambridge (GB); E-Chiang LEE, Cambridge (GB)

(21) Appl. No.: 19/282,256

(22) Filed: Jul. 28, 2025

Related U.S. Application Data
(63) Continuation of application No. 16/471,161, now abandoned.

(30) Foreign Application Priority Data
Dec. 20, 2016 (GB) 1621782.0
Feb. 13, 2017 (GB) 1702338.3
Feb. 13, 2017 (GB) 1702339.1
Feb. 24, 2017 (GB) 1703071.9
Jun. 20, 2017 (GB) 1709818.7
Jun. 20, 2017 (TW) 106120562
Jun. 20, 2017 (TW) 106120563
Jun. 20, 2017 (TW) 106120564
Jun. 20, 2017 (WO) PCT/GB2017/051794
Jun. 20, 2017 (WO) PCT/GB2017/051795
Jun. 20, 2017 (WO) PCT/GB2017/051796
Aug. 9, 2017 (TW) 106126908
Aug. 9, 2017 (WO) PCT/GB2017/052352

(51) Int. Cl. C07K 16/28; A61K 38/00; A61K 39/00; A61P 35/00

(52) U.S. Cl. CPC C07K 16/2818; A61P 35/00; C07K 16/2827; A61K 38/00; A61K 2039/507; C07K 2317/21; C07K 2317/31; C07K 2317/32; C07K 2317/33; C07K 2317/55; C07K 2317/565; C07K 2317/64; C07K 2317/732; C07K 2317/75; C07K 2317/76; C07K 2317/92

(57) ABSTRACT

Multispecific antibody having a binding site for ICOS and a binding site for a second antigen, e.g., an immune checkpoint molecule such as PD-L1. Use of the multispecific antibody in immuno-oncology, including for treatment of solid tumours.

### RELATED APPLICATIONS

This application is a continuation of U.S. patent application Ser. No. 16/471,161, filed Jun. 19, 2019, which is a national stage filing under 35 U.S.C. § 371 of International Application No. PCT/GB2017/053826, filed Dec. 19, 2017, which claims priority to Great Britain Patent Application Nos. 1621782.0, filed Dec. 20, 2016, 1702339.1, filed Feb. 13, 2017, 1702338.3, filed Feb. 13, 2017, 1703071.9, filed Feb. 24, 2017, Taiwanese Patent Application No. 106120563, filed Jun. 20, 2017, International Application No. PCT/GB2017/051794, filed Jun. 20, 2017, British Patent Application No. 1709818.7, filed Jun. 20, 2017, International Application No. PCT/GB2017/051795, filed Jun. 20, 2017, Taiwanese Patent Application No. 106120562, filed Jun. 20, 2017, International Application No. PCT/GB2017/051796, filed Jun. 20, 2017, Taiwanese Patent Application No. 106120564, filed Jun. 20, 2017, International Application No. PCT/GB2017/052352, filed Aug. 9, 2017, and Taiwanese Patent Application No. 106126908, filed Aug. 9, 2017. The contents of these applications are each incorporated herein by reference.

### SEQUENCE LISTING

The instant application contains a Sequence Listing which has been submitted electronically in XML format and is hereby incorporated by reference in its entirety. Said XML file, created on Jul. 28, 2025, is named 764716_SA9-622USCON_ST26.xml and is 609,150 bytes in size.

### FIELD OF THE INVENTION

This invention relates to antigen-binding molecules that bind cell surface receptors involved in regulation of the immune response. It relates to antibodies for use in stimulating a patient's immune system, especially the effector T cell response, and has applications in the field of immuno-oncology, especially treatment of tumours.

### BACKGROUND

An adaptive immune response involves activation, selection, and clonal proliferation of two major classes of lymphocytes termed T cells and B cells. After encountering an antigen, T cells proliferate and differentiate into antigen-specific effector cells, while B-cells proliferate and differentiate into antibody-secreting cells. T cell activation is a multi-step process requiring several signalling events between the T cell and an antigen-presenting cell (APC). For T cell activation to occur, two types of signals must be delivered to a resting T cell. The first type is mediated by the antigen-specific T cell receptor (TCR), and confers specificity to the immune response. The second signal, a costimulatory signal, regulates the magnitude of the response and is delivered through accessory receptors on the T cell.

A primary costimulatory signal is delivered through the activating CD28 receptor upon engagement of its ligands B7-1 or B7-2. In contrast, engagement of the inhibitory CTLA-4 receptor by the same B7-1 or B7-2 ligands results in attenuation of a T cell response. Thus, CTLA-4 signals antagonise costimulation mediated by CD28. At high antigen concentrations, CD28 costimulation overrides the CTLA-4 inhibitory effect. Temporal regulation of the CD28 and CTLA-4 expression maintains a balance between activating and inhibitory signals and ensures the development of an effective immune response, while safeguarding against the development of autoimmunity.

Programmed death-1 (PD-1) is a 50-55 kDa type I transmembrane receptor that is a member of the CD28 family. PD-1 is involved in the regulation of T-cell activation and is expressed on T cells, B cells, and myeloid cells. Two ligands for PD-1, PD ligand 1 (PD-L1) and ligand 2 (PD-L2) have been identified and have costimulatory features.

Programmed cell death 1 ligand 1 (PD-L1), also known as cluster of differentiation (CD274) or B7 homolog 1 (B7-H1), is a member of the B7 family that modulates activation or inhibition of the PD-1 receptor. The open reading frame of PD-L1 encodes a putative type 1 transmembrane protein of 290 amino acids, which includes two extracellular Ig domains (an N-terminal V-like domain and an Ig C-like domain), a hydrophobic transmembrane domain and a cytoplasmic tail of 30 amino acids. The 30 amino acid intracellular (cytoplasmic) domain contains no obvious signalling motifs, but does have a potential site for protein kinase C phosphorylation. The complete amino acid sequence for PD-L1 can be found in NCBI Reference Sequence: NP_054862.1 (SEQ ID NO: 1), which refers to many journal articles [1]. The PD-L1 gene is conserved in chimpanzee, Rhesus monkey, dog, cow, mouse, rat, chicken, and zebrafish. The murine form of PD-L1 bears 69% amino acid identity with the human form of PD-L1, and also shares a conserved structure.

In humans, PD-L1 is expressed on a number of immune cell types including activated and anergic/exhausted T cells, on naive and activated B cells, as well as on myeloid dendritic cells (DC), monocytes and mast cells. It is also expressed on non-immune cells including islets of the pancreas, Kupffer cells of the liver, vascular endothelium and selected epithelia, for example airway epithelia and renal tubule epithelia, where its expression is enhanced during inflammatory episodes. PD-L1 expression is also found at increased levels on a number of tumours, such as breast (e.g., triple negative breast cancer and inflammatory breast cancer), ovarian, cervical, colon, colorectal, lung (e.g., non-small cell lung cancer), renal (e.g., renal cell carcinoma), gastric, oesophageal, bladder, hepatocellular cancer, squamous cell carcinoma of the head and neck (SCCHN) and pancreatic cancer, melanoma and uveal melanoma.

PD-1/PD-L1 signalling is believed to serve a critical non-redundant function within the immune system by negatively regulating T cell responses. This regulation is involved in T cell development in the thymus, in regulation of chronic inflammatory responses and in maintenance of both peripheral tolerance and immune privilege. It appears that upregulation of PD-L1 may allow cancers to evade the host immune system and, in many cancers, the expression of PD-L1 is associated with reduced survival and an unfavourable prognosis. Therapeutic monoclonal antibodies that are able to block the PD-1/PD-L1 pathway may enhance anti-tumoural immune responses in patients with cancer. Published clinical data suggest a correlation between clinical responses with tumoural membranous expression of PD-L1 and a stronger correlation between lack of clinical responses and a lack of PD-L1 protein localised to the membrane [2, 3]. Thus, PD-L1 expression in tumours or tumour-infiltrating leukocytes is a candidate molecular marker for use in selecting patients for immunotherapy, for example, immunotherapy using anti-PD-L1 antibodies [4]. Patient enrichment based on surface expression of PD-L1 may significantly enhance the clinical success of treatment with drugs targeting the PD-1/PD-L1 pathway. There is also evidence of an ongoing immune response, such as the tumour infiltrating CD8+ T cells, or the presence of signature of cytokine activation, such as IFNγ.

Further evidence of PD-L1 expression and correlation to disease will emerge from the numerous ongoing clinical trials. Atezolizumab is the most advanced anti-PD-L1 antibody in development, and Phase II trials showed therapeutic effects in metastatic urothelial carcinoma and NSCLC, particularly in patients with PD-L1⁺ immune cells in the tumour microenvironment [5, 6]). Recent results from a Phase III trial of 1225 patients with NSCLC showed improved survival in patients taking atezolizumab, compared with chemotherapy, regardless of tumour expression of PD-L1 (Rittmeyer et al., 2017, The Lancet, 389(10066), 255-265).

Another member of the CD28 gene family, ICOS (Inducible T cell Co-Stimulator), was identified in 1999 [7]. It is a 55 kDa transmembrane protein, existing as a disulphide linked homodimer with two differentially glycosylated subunits. ICOS is exclusively expressed on T lymphocytes, and is found on a variety of T cell subsets. It is present at low levels on naïve T lymphocytes but its expression is rapidly induced upon immune activation, being upregulated in response to pro-inflammatory stimuli such as on engagement of TCR and co-stimulation with CD28 [8, 9]. ICOS plays a role in the late phase of T cell activation, memory T cell formation and importantly in the regulation of humoral responses through T cell dependent B cell responses [10, 11]. Intracellularly, ICOS binds PI3K and activates the kinases phosphoinositide-dependent kinase 1 (PDK1) and protein kinase B (PKB). Activation of ICOS prevents cell death and upregulates cellular metabolism. In the absence of ICOS (ICOS knock-out) or in the presence of anti-ICOS neutralising antibodies there would be a suppression of pro-inflammatory responses.

ICOS binds to ICOS ligand (ICOSL) expressed on B-cells and antigen presenting cells (APC) [12, 13]. As a co-stimulatory molecule it serves to regulate TCR mediated immune responses and antibody responses to antigen. The expression of ICOS on T regulatory cells may be important, as it has been suggested that this cell type plays a negative role in immunosurveillance of cancer cells—there is emerging evidence for this in ovarian cancer [14]. Importantly, ICOS expression has been reported to be higher on intratumoural regulatory T cells (TRegs) compared with CD4+ and CD8+ effector cells that are present in the tumour microenvironment. Depletion of TRegs using antibodies with Fc-mediated cellular effector function has demonstrated strong anti-tumour efficacy in a pre-clinical model [15]. Mounting evidence implicates ICOS in an anti-tumour effect in both animal models as well as patients treated with immune-checkpoint inhibitors. In mice deficient in ICOS or ICOSL the anti-tumor effect of anti-CTLA4 therapy is diminished while in normal mice ICOS ligand increases the effectiveness of anti-CTLA4 treatment in melanoma and prostate cancer [17]. Furthermore, in humans a retrospective study of advanced melanoma patients showed increased levels of ICOS following ipilimumab (anti-CTLA4) treatment [18]. In addition, ICOS expression is upregulated in bladder cancer patients treated with anti-CTLA4 [19]. It has also been observed that in cancer patients treated with anti-CTLA4 therapy the bulk of tumour specific IFNγ producing CD4 T-cells are ICOS positive while sustained elevation of ICOS positive CD4 T cells correlates with survival [18, 19, 20].

WO2016/120789 described anti-ICOS antibodies and proposed their use for activating T cells and for treating cancer, infectious disease and/or sepsis. A number of murine anti-ICOS antibodies were generated, of which a sub-set were reported to be agonists of the human ICOS receptor. The antibody “422.2” was selected as the lead anti-ICOS antibody and was humanised to produce a human “IgG4PE” antibody designated “H2L5”. H2L5 was reported to have an affinity of 1.34 nM for human ICOS and 0.95 nM for cynomolgus ICOS, to induce cytokine production in T cells, and to upregulate T cell activation markers in conjunction with CD3 stimulation. However, mice bearing implanted human melanoma cells were reported to show only minimal tumour growth delay or increase in survival when treated with H2L5 hIgG4PE, compared with control treated group. The antibody also failed to produce significant further inhibition of tumour growth in combination experiments with ipilimumab (anti-CTLA-4) or pembrolizumab (anti-PD-1), compared with ipilimumab or pembrolizumab monotherapy. Finally, in mice bearing implanted colon cancer cells (CT26), low doses of a mouse cross reactive surrogate of H2L5 in combination with a mouse surrogate of ipilimumab or pembrolizumab only mildly improved overall survival compared with anti-CTL4 and anti-PD1 therapy alone. A similar lack of strong therapeutic benefit was shown in mice bearing implanted EMT6 cells. 5

WO2016/154177 described further examples of anti-ICOS antibodies. These antibodies were reported to be agonists of CD4+ T cells, including effector CD8+ T cells (TEff), and to deplete T regulator cells (TRegs). Selective effects of the antibodies on TEff vs TReg cells were described, whereby the antibodies could preferentially deplete TRegs while having minimal effect on TEffs that express a lower level of ICOS. The anti-ICOS antibodies 10 were proposed for use in treating cancer, and combination therapy with anti-PD-1 or anti-PD-L1 antibodies was described.

Although there has been immense progress in the field of immuno-oncology in recent years, current response rates of immuno-oncology drugs remain low. For example, the response rate for the anti-PD-1 antibody nivolumab in melanoma is around 30%, and the response rate for the anti-PD-L1 atezolizumab in its Phase II clinical trial in urothelial carcinoma was around 15% overall in patients regardless of PD-L1 expression or 26% in patients with PD-L1 expressing tumours. Efforts to increase efficacy of immuno-oncology treatment have included combining multiple drugs, for example combinations of antibodies and traditional chemotherapeutic agents or radiation, and the combined use of drugs targeting different immune checkpoint inhibitors. A combination of nivolumab (anti-PD-1) and ipilimumab (anti-CTLA-4) has shown efficacy in previously untreated cases of melanoma, with headline response rates and overall survival being encouraging [21]. However, although combination therapy may generate new or enhanced biological effects in vivo, this carries an associated risk of negative drug interactions and new or worsened side-effects. Immune checkpoint inhibitor therapy is already associated with immune-related adverse events, including neurological events ranging from mild headache to life-threatening encephalitis [22]. Further, on a practical level, treatment regimens involving combinations of multiple therapeutic agents have the drawbacks of complex administration regimens and high cost.

### SUMMARY OF THE INVENTION

The present invention relates to antigen-binding molecules that comprise multiple antigen-binding sites (“multispecific antigen-binding molecules”), including an antigen-binding site for ICOS and an antigen-binding site for another target antigen, e.g., PD-L1.

Both ICOS and PD-L1 are expressed following primary T cell activation. PD-L1 negatively regulates T cell activation, and inhibition of PD-L1 signalling has been clinically validated as an approach to upregulate the T cell immune response against tumour cells. In context, parallel depletion of ICOS-high Tregs and stimulation of ICOS-low effector T cells can enhance T cell activation to promote anti-tumour activity.

A multispecific antigen-binding molecule that blocks the negative regulatory activity of PD-L1 on PD1+ T cells and enhances T cell activation by delivering a positive signal through ICOS offers therapeutic potential in treating cancer and other conditions in which it is desirable to upregulate the T cell immune response. The fate of T cells in the tumour microenvironment and in tumour-draining lymph nodes is influenced by a balance of inhibitory and activatory receptors, and a molecule that binds and inhibits PD-L1 while acting as an ICOS agonist may effectively turn a negative signal (from the inhibitory PD-L1 receptor) into a positive signal (from the ICOS co-activatory receptor). The immune synapse between a T cell and an antigen-presenting cell (APC) or tumour cell can be envisaged as a receptor-dense space in which the balance of receptor occupancy determines signalling within the T cell, this receptor occupancy being governed by the identity and concentration of receptors being presented on the surface of the engaging APC/tumour cell. A multispecific molecule bearing a binding site for ICOS and a binding site for PD-L1 may act directly at this immune synapse to change the balance of signals received by T cells, shifting the balance towards activation of TEffs. Combination of anti-PD-L1 and anti-ICOS in one multispecific antigen-binding molecule, rather than separate antigen-binding molecules, provides a single agent that can act as a molecular switch. The multispecific molecule may cross-link ICOS and PD-L1 on different cells (FIG. 1).

In addition to binding its two cognate antigens, a multi-specific antigen-binding molecule may incorporate other moieties such as antibody effector regions to recruit cell-killing functions, which may further tip the immune balance towards T cell activation and killing of cancer cells, e.g., via depletion of TRegs which highly express ICOS on the cell surface and/or depletion of cancer cells expressing PD-L1. A bispecific antibody binding to ICOS and PD-L1 may trigger ADCC towards PD-L1+ immunosuppressive cells (e.g., MDSC, tumour cells) and/or ADCC towards ICOS+ immunosuppressive cells (e.g., Tregs).

A multispecific antigen-binding molecule according to the present invention may be an antibody (e.g., a bispecific or dual-binding antibody) that binds ICOS and another target antigen. Numerous multispecific antibody formats are possible, and many examples are provided herein. The antibody may be bivalent for both target antigens. For example, the antibody may be a FIT-Ig comprising two ICOS-binding Fab domains and two PD-L1 binding domains (e.g., as illustrated in FIG. 2). Alternatively, the antibody may be a mAb² comprising two ICOS-binding Fab domains and an Fc region comprising two binding sites for PD-L1 (i.e., a PD-L1 binding Fcab), as illustrated in FIG. 3. Examples of ICOS antibodies and PD-L1 antibodies sequences, including VH and VL domain sequences, are set out herein and may be included in the multispecific antibodies.

A multispecific antigen-binding molecule that binds ICOS and PD-L1 may increase response rates of tumours that are already responsive to PD-L1 or ICOS monotherapy, increasing the proportion of patients in whom an anti-tumour response is observed and potentially improving the level of response, reducing tumour growth and extending survival compared with monotherapy. Some tumours are unresponsive to either anti-ICOS or anti-PD-L1 antibody, but may respond to a multispecific antibody that binds ICOS and PD-L1. Anti-ICOS/anti-PD-L1 bispecific binding molecules may also be used for inducing long term memory to antigens, e.g., tumour antigens, thereby providing protection against tumour regrowth. Thus, the multispecific approach described here offers advantages in improving response rates, duration of response, and patient survival, in the context of cancer therapy. Furthermore, a multispecific antigen-binding molecule can be administered to patients using simpler treatment regimens compared with multiple separate formulations of different therapeutic agents.

### BRIEF DESCRIPTION OF THE DRAWINGS

FIG. 1. Redirecting modulation of an immune checkpoint. The multispecific antigen-binding molecule effects a simultaneous blockade of PD-L1 receptors on antigen-presenting cells (APC) or tumour cells and agonism of the ICOS receptor on T effector cells, switching a negative regulatory signal to a positive regulatory signal at the T cell immune synapse.

FIG. 2. FIT-Ig format of bispecific antibodies that bind ICOS and PD-L1. (i) Assembled FIT-Ig antibody (ii) Polypeptide chains included in FIT-Ig antibody. Construct #1 is a polypeptide containing, in the N to C direction, the light variable (VL) and light constant (CL) regions of antibody “A”, fused to the heavy variable (VH) and heavy constant regions (CH1, CH2, CH3) of antibody “B”. Preferably, no linker is included between the CL and VH_{B} domain. Construct #2 is a polypeptide fusion of the heavy variable (VH) region and CH1 of antibody “A”. Construct #3 is a polypeptide fusion of the light variable (VL) and light constant (CL) regions of antibody “B”. The FIT-Ig may be constructed with antibody “A” being anti-ICOS and antibody “B” being anti-PD-L1, or with antibody “A” being anti-PD-L1 and antibody “B” being anti-ICOS.

FIG. 3. Example mAb² IgG format of bispecific antibody that binds ICOS and PD-L1. The mAb² is a homodimeric IgG comprising two anti-ICOS Fab and two CH3 domains each having three binding loops forming a PD-L1 binding site (the anti-PD-L1 Fcab region).

FIG. 4 (A) STIM001 and STIM003 mAb² binding to recombinant human ICOS protein. Data representative of three experiments. (B) STIM001 and STIM003 mAb² binding to recombinant mouse ICOS protein. Data representative of three experiments. (C) Human STIM001 and STIM003 mAb² binding to recombinant human PD-L1 protein. Data representative of three experiments. (D) Mouse STIM001 and STIM003 mAb² binding to recombinant mouse PD-L1 protein. Data representative of 3 experiments.

FIG. 5 Results of ICOS FACS binding assay described in Example 4. A) mAb² binding to human ICOS expressed on CHO cells. Data representative of 3 experiments. B) mAb² binding to mouse ICOS expressed on CHO cells. Data representative of 3 experiments.

FIG. 6 Results of human PD-L1 FACS binding assay described in Example 4. A) Human PD-L1-binding FACS with anti-human IgG detection. Binding profiles of STIM001_289, STIM003_289 and IgG1_289 anti-PD-L1 mAb²s and respective mAb controls. B) Human PD-L1-binding FACS with bound human ICOS labelled AlexaFluor 647 detection. Binding profiles of STIM001_289, STIM003_289 and IgG1_289 anti-PD-L1 mAb²s and respective mAb controls.

FIG. 7 Results of mouse PD-L1 FACS binding assay described in Example 4. A) Mouse PD-L1-binding FACS with anti-human IgG detection. Binding profiles of STIM001_457, STIM003_457 and IgG1_438 anti-PD-L1 mAb²s and respective monospecific mAb controls. B) Mouse PD-L1-binding FACS with bound human ICOS labelled AlexaFluor 647 detection. Binding profiles of STIM001_457, STIM003_457 and IgG1_438 anti-PD-L1 mAb²s and respective monospecific mAb controls.

FIG. 8 (A) Human STIM001 and STIM003 mAb² Fc engagement to human FcγRIIIa on effector cells, as described in Example 5b. Data representative of 3 experiments. (B) Mouse STIM001 and STIM003 mAb² Fc engagement to FcγRIIIa on effector cells, as described in Example 5b. Data representative of 3 experiments.

FIG. 9 Concentration-dependent study of STIM001_289 and STIM003_289 mediated ADCC on ICOS-transfected CCRF-CEM cells using freshly isolated NK cells as effector cells for 3 independent donors (panels A, B and C), as described in Example 5c. The effector cells and target cells (effector:target ratio of 5:1) were incubated together with antibody for 4 hours. Dye release from lysed target cells was measured as described in the kit manufacturer's instructions. Lysis buffer was used to determine the 100% release. Basal killing (no Ab) is indicated by a dotted line at the bottom of each graph.

FIG. 10A) Results of mouse ICOS-Ligand neutralisation HTRF assay with mouse ICOS receptor described in Example 6. Neutralisation profiles of STIM001_289, STIM001_457, STIM003_289 and STIM003_457 mAb². Data representative of three experiments. B) Results of human ICOS-Ligand neutralisation HTRF assay with human ICOS receptor described in Example 6. Neutralisation profiles of STIM001_289, STIM001_457, STIM003_289 and STIM003_457 mAb². Data representative of three experiments.

FIG. 11 Results of PD-L1 neutralisation assay described in Example 7. A) Human PD-L1 Neutralisation FACS to human PD1. Binding profiles of STIM001_289, STIM003_289 and IgG1_289 anti-PD-L1 mAb²s and respective mAb controls. B) Human PD-L1 Neutralisation FACS to human CD80. Binding profiles of STIM001_289, STIM003_289 and IgG1_289 anti-PD-L1 mAb²s and respective mAb controls.

FIG. 12 A) Data from mouse PD-L1 neutralisation assay (FACS) to mouse PD1 as described in Example 7. Binding profiles of STIM001_457, STIM003_457 and IgG1_438 anti-PD-L1 mAb²s and respective controls. B) Data from mouse PD-L1 neutralisation assay (FACS) to mouse CD80. Binding profiles of STIM001_457, STIM003_457 and IgG1_438 anti-PD-L1 mAb²s and respective controls.

FIG. 13 Concentration-dependent study of STIM001_289 and STIM003_289 vs STIM001 and STIM003 agonist effect on isolated human T-cells co-stimulated with CD3/CD28 dynabeads for 3-days. IFN-γ production was used as a read-out of ICOS agonism. All antibodies were tested plate-bound and compared to their isotype controls. Mean±SD values of technical replicates as well as non-linear regression curves (variable slope, 4-parameter) are shown for 1 donor (278) in the panels A and B. In panel C is shown an example set of data comparing the levels of IFN-γ (mean value) induced at one given dose (3.3 μM) for all 4 donors. Each dot represents an independent donor identifiable by its number and the median of 4 donors is marked by a line. Significance was assessed using Friedman statistic test and p-values are indicated on the graph.

FIG. 14 Concentration-dependent study of STIM001_289, STIM003_289 and IgG1_289 vs PD-L1 AbV effect on cytokine production by CD45RO⁺ T-cells co-culture with autologous monocytes in presence of CD3 antibody (TCR activation). In this assay, as described in Example 9, IFN-γ production is used as a read-out of the neutralisation of PD-1/PD-L1 interaction by the test antibody. All antibodies were compared to the isotype control (IgG1). (A) Raw data of one independent donor (288) is shown in the upper panel. The basal IFN-γ levels (mono/T-cells dotted line) was used to normalise the values and calculate the fold increase in IFN-γ. (B) In the lower panel is shown an example of data comparing the increase in IFN-γ induced at one given dose (10 nM) for all 7 donors. Each dot represents an independent donor identifiable by its number and the median is marked by a line. Significance was assessed using Friedman statistic test (*<0.05 and **<0.01).

FIG. 15 Effect of STIM001_457 and STIM003_457 bispecific antibodies in the J558 syngeneic tumour study described in Example 10. Each treatment group is represented by a “spider plot” showing the tumour size of individual animals (n=10 or n=8 per group). (A) Saline treated group. Both bispecific antibodies demonstrated significant anti-tumour efficacy with 5 out 8 animals treated with STIM001_457 (B) and 4 out of 8 of those treated with STIM003_457 (C) cured from their disease at day 37. The number of animals cured of their disease is indicated on the bottom right of the respective graphs. Scheduled dosing days are indicated by dotted lines (day 11, 15, 18, 22, 25 and 29).

FIG. 16 Kaplan-Meier survival curves/time on study of the % mice surviving after the different treatments described in Example 10. Median survival time of animals on saline (open square) and STIM003_457 (triangle) were 18 and 27.5 days, respectively. Median survival for STIM001_457 (black circle) was not reached.

FIG. 17 Data from CT26 in vivo efficacy study described in Example 11a. Each treatment group is represented by a “spider plot” showing the tumour size of individual animals (n=10 per groups). For each group, the number of animals cured of their disease is indicated on the bottom left of the respective graphs. Dosing was on days 6, 8, 10, 13, 15 and 17, and dosing time is indicated by the shaded area. (A) Saline; (B) IgG1_457 LAGA control; (C) STIM003_457; (D) STIM001_457.

FIG. 18 Kaplan Meier plot for CT26 study described in Example 11a. Circles: saline control. Squares: IgG1_487 control. Up triangles: STIM003_457. Down triangles: STIM001_457.

FIG. 19 Treatment with ICOS/PD-L1 antibody results in a long-term anti-tumour memory response in animals previously cured from CT26 tumours. As described in Example 11b, mice cured from CT26 colon cancer were rechallenged s.c. in the left side of their abdomen with either 2.5×10⁵ EMT-6 cells (n=4 mice per group) or 1×10⁵ CT26 cells (n=5 mice per group). The spider plots show the tumour growth during 20 days following (A) EMT-6 or (B) CT26 cell inoculation.

FIG. 20 Results of A20 in vivo efficacy study described in Example 12. Each treatment group is represented by a “spider plot” showing the tumour size of individual animals (n=10 per group). For each group, the number of animals cured of their disease is indicated on the bottom left of the respective graph. Dosing was on days 8, 11, 15, 18, 22 and 25. (A) saline; (B) IgG_457; C) STIM003_457; and STIM001_457.

FIG. 21 Results of A20 in vivo efficacy study described in Example 12. The humane endpoint survival statistics were calculated from the Kaplan-Meier curves using GraphPad Prism V7.0. This approach was used to determine if specific treatments were associated with improved survival.

FIG. 22 Data from EMT6 in vivo efficacy study described in Example 13. Each treatment group is represented by a “spider plot” showing the tumour size of individual animals (n=10 per group). A) Saline B) Anti-PD-L1 mAb² control antibody C) STIM003_457 D) STIM001_457. For each group, the number of animals cured of their disease is indicated on the bottom left of the respective graph. Dosing was on days 6, 9, 13, 16, 20 and 23.

FIG. 23 Survival (time on study) for the animals treated with saline (black circles), anti-PD-L1 mAb² control antibody (squares), STIM003_457 (up triangles), or STIM001_457 (down triangles), as described in Example 13. Both ICOS/PD-L1 bispecific antibodies significantly improved the overall survival of animals compared with those treated with saline.

FIG. 24 Bispecific efficacy in the EMT6 model described in Example 13. A) IgG1 LAGA hybrid control mAb² antibody with anti-PD-L1 457 Fcab; B) combination of STIM003 and anti-PD-L1 antibody (mouse IgG2a format); C) STIM001_457 bispecific antibody; D) STIM003_457 bispecific antibody.

FIG. 25 Kaplan Meier (humane endpoint) showing superior efficacy of treatment with the PD-L1/ICOS bispecific antibodies (down triangles for STIM001_457, up triangles for STIM003_457) compared with combined administration of anti-PD-L1 monospecific antibody and anti-ICOS monospecific antibody (black diamonds) in the EMT6 model described in Example 13. Data from saline control treatments shown in closed circles. Data from IgG1 LAGA hybrid control mAb² antibody with anti-PD-L1 457 Fcab shown in open squares.

FIG. 26 Representative example from two independent experiments in the PD-L1 dependent ICOS agonism assay reported in Example 14. (A) BSA. (B) B7-H1-Fc. (C) Goat anti-human IgG Fcg fragment specific F(ab′)2.

FIG. 27 Identification of four different quadrants on dot plot graph for mAb² antibodies in a PD-L1/ICOS cell recruitment assay by flow cytometry.

FIG. 28 Titration of mAb² and monospecific antibodies in a PD-L1/ICOS cell recruitment assay by flow cytometry. CHO human PD-L1 and CHO human ICOS were stained with CellTrace™ Far Red and CellTrace™ Violet respectively and incubated together in presence of antibodies for an hour prior to the detection of fluorescence and identification of double positive population. Data shown are representative of two independent experiments.

FIG. 29 FACS analysis revealed that STIM003_457 and STIM001_457 significantly deplete regulatory T-cells (T_{Regs}) and increase effector cell:T_{Regs} ratio in tumour. Animals (BALB/c mice) were dosed with saline, STIM003_457 or STIM001_457 (n=8 per group) on days 13 and 15 days post-implantation of CT-26.WT tumour cells s/c. Proportion of T_{Regs} of total live tumour cells (A) and of total CD4+ cells (B) were significantly decreased in response to both antibodies compared to saline. Ratios of CD4+ effector cells to T_{Regs} (C), and of CD8+ cells to T_{Regs} (D) are significantly increased compared to the control. Kruskal-Wallis test was performed followed by post-hoc Dunn's test. *p<0.05. **p<0.01. ***p<0.001. ****p<0.0001.

FIG. 30 FACS analysis shows that STIM003_457 and STIM001_457 have little effect on regulatory T-cell (T_{Regs}) levels in the spleen of a CT-26.WT tumour-bearing mouse. STIM003_457 shows a marginal T_{Regs} depletion as a percentage of total live cells, but STIM001_457 has no effect (A). No clear changes were observed when looking at the effect of the bispecific on T_{Regs} as a percentage of total CD4⁺ cells (B). No significant changes are seen in CD4⁺ effector cell to T_{Regs} (C), and CD8⁺ cell to T_{Regs} ratios (D). Kruskal-Wallis test was performed followed by post-hoc Dunn's test. *p<0.05.

FIG. 31 FACS analysis demonstrates increase in ICOS-Ligand (ICOS-L) expression on B-cells in the spleens of CT26-WT tumour-bearing mice dosed with STIM003_457 and STIM001_457. When compared to saline, both bispecific antibodies caused a significant increase in the percentage of B-cells expressing ICOS-L in the spleen (A). A significant increase in mean fluorescence intensity (relative expression) of ICOS-L on B-cells was also seen in both bi-specific groups compared to the saline group (B). Kruskal-Wallis test was performed followed by post-hoc Dunn's test. *p<0.05. ****p<0.0001.

FIG. 32 Graph showing the average weight of the mice over the 46 days for the different treatment groups. Vertical lines indicate the day the animals were dosed IP. Note that the small decrease in average weight observed from day 35 for group 3 (aCTLA-4 monotherapy) and group 7 (STIM003/aPDL1 combination) is due to some animals coming out of the study for tumour size.

FIG. 33A to G spider plot graphs showing the CT26 tumour size of individual animals over time in response to the different treatments. The “triple combination” of antibodies (against ICOS, PD-L1 and PD1 or CTLA-4) were associated with the most pronounced anti-tumour response in the CT26 model. Vertical lines indicate the day the animals were dosed IP. For the combination the antibodies were injected concomitantly. The numbers at the bottom right end of each graph indicate the number of animals still on study on day 46 (40 days after the treatments were initiated).

### DETAILED DESCRIPTION

### Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In the specification and claims, the term “about” is used to modify, for example, the quantity of an ingredient in a composition, concentration, volume, process temperature, process time, yield, flow rate, pressure, and like values, and ranges thereof, employed in describing the embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an anti-hPD-L1 antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The term “antibody”, “immunoglobulin” or “lg” may be used interchangeably herein and means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)₂, and Fμ fragments), single chain Fμ (scFv) mutants, multispecific antibodies such as bispecific antibodies (including dual binding antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, hIgG2, mIgG2A, mIgG2B, IgG3, IgG4, IgA1 and IgA2. In mammals there are two types of immunoglobulin light chains, λ and κ. The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The antibodies described herein may be oligoclonal, polyclonal, monoclonal (including full-length monoclonal antibodies), camelised, chimeric, CDR-grafted, multi-specific, bi-specific (including dual-binding antibodies), catalytic, chimeric, humanized, fully human, anti-idiotypic, including antibodies that can be labelled in soluble or bound form as well as fragments, variants or derivatives thereof, either alone or in combination with other amino acid sequences provided by known techniques. An antibody may be from any species. Antibodies described herein can be naked or conjugated to other molecules such as toxins, radioisotopes, etc.

The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g., the complementarity determining regions (CDRs)). The antigen binding region can be derived from any animal species, such as rodents (e.g., rabbit, rat or hamster) and humans. Preferably, the antigen binding region will be of human origin. Antigen binding fragments described herein can include single-chain Fvs (scFv), single-chain antibodies, single domain antibodies, domain antibodies, Fv fragments, Fab fragments, F(ab′) fragments, F(ab′)₂ fragments, antibody fragments that exhibit the desired biological activity, disulfide-stabilised variable region (dsFv), dimeric variable region (diabody), anti-idiotypic (anti-Id) antibodies (including, e.g., anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognized by Fc receptors (FcR) found on certain types of cells. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals. The monoclonal antibodies herein can include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies that exhibit the desired biological activity.

The term “humanized antibody” refers to a subset of chimeric antibodies in which a “hypervariable region” from a non-human immunoglobulin (the donor antibody) replaces residues from a hypervariable region in a human immunoglobulin (recipient antibody). In general, a humanized antibody will include substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more substitutions that improve antibody performance, such as binding affinity, isomerization, immunogenicity, etc.

The term “bispecific antibody” means an antibody which comprises specificity for two target molecules, and includes formats such as bispecific IgG (optionally wherein the IgG has a common light chain), DVD-Ig (see DiGiammarino et al., “Design and generation of DVD-Ig™ molecules for dual-specific targeting”, Meth. Mo. Biol., 2012, 889, 145-156), mAb² (see WO2008/003103, the description of the mAb² format is incorporated herein by reference), FIT-Ig (see WO2015/103072, the description of the FIT-Ig scaffold is incorporated herein by reference), mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. For a review of bispecific formats, see Spiess, C., et al., Mol. Immunol. (2015). In another embodiment, the bispecific molecule comprises an antibody which is fused to another non-Ig format, for example a T-cell receptor binding domain; an immunoglobulin superfamily domain; an agnathan variable lymphocyte receptor; a fibronectin domain (e.g., an Adnectin™); an antibody constant domain (e.g., a CH3 domain, e.g., a CH2 and/or CH3 of an Fcab™) wherein the constant domain is not a functional CH1 domain; an scFv; an (scFv) 2; an sc-diabody; an scFab; a centyrin and an epitope binding domain derived from a scaffold selected from CTLA-4 (Evibody™); a lipocalin domain; Protein A such as Z-domain of Protein A (e.g., an Affibody™ or SpA); an A-domain (e.g., an Avimer™ or Maxibody™); a heat shock protein (such as and epitope binding domain derived from GroEI and GroES); a transferrin domain (e.g., a trans-body); ankyrin repeat protein (e.g., a DARPin™); peptide aptamer; C-type lectin domain (e.g., Tetranectin™); human γ-crystallin or human ubiquitin (an affilin); a PDZ domain; scorpion toxin; and a kunitz type domain of a human protease inhibitor.

In one embodiment, the bispecific antibody is a mAb². A mAb² comprises a V_{H} and V_{L} domain from an intact antibody, fused to a modified constant region, which has been engineered to form an antigen-binding site, known as an “Fcab”. The technology behind the Fcab/mAb² format is described in more detail in WO2008/003103, and the description of the mAb² format is incorporated herein by reference.

In one embodiment, a “bispecific antibody” does not include a FIT-Ig format. In one embodiment, a “bispecific antibody” does not include a mAb² format. In one embodiment, a “bispecific antibody” does not include either a FIT-Ig format or a mAb² format.

In another embodiment, the bispecific antibody is a “dual binding antibody”. As used herein, the term “dual binding antibody” is a bispecific antibody wherein both antigen-binding domains are formed by a V_{H}/V_{L} pair, and includes FIT-Ig (see WO2015/103072, incorporated herein by reference), mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv and scFv4-Ig.

The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the VH (CDRH1, CDRH2, CDRH3), and three in the VL (CDRL1, CDRL2, CDRL3). These regions of the heavy and light chains of an antibody confer antigen-binding specificity to the antibody. CDRs may be defined according to the Kabat system (see Kabat, E. A. et al., 1991, “Sequences of Proteins of Immunological Interest”, 5th edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues. The term “specifically binds to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA).

An antibody or a fragment thereof that specifically binds to a hPD-L1 antigen may be cross-reactive with related antigens. Preferably, an antibody or a fragment thereof that specifically binds to a hPD-L1 antigen does not cross-react with other antigens (but may optionally cross-react with PD-L1 of a different species, e.g., rhesus, or murine). An antibody or a fragment thereof that specifically binds to a hPD-L1 antigen can be identified, for example, by immunoassays, BIAcore™, or other techniques known to those of skill in the art. An antibody or a fragment thereof binds specifically to a PD-L1 antigen when it binds to a hPD-L1 antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times (such as more than 15 times, more than 20 times, more than 50 times or more than 100 times) background. See, e.g., Paul, ed., 1989, Fundamental Immunology Second Edition, Raven Press, New York at pages 332-336 for a discussion regarding antibody specificity

The term “aliphatic amino acid” means that the amino acid R groups are nonpolar and hydrophobic. Hydrophobicity increases with increasing number of C atoms in the hydrocarbon chain. Glycine, Alanine, Valine, Leucine and Isoleucine are aliphatic amino acids.

The term “aromatic amino acid” means that the amino acid R groups contain an aromatic ring system. Phenylalanine, Tyrosine and Tryptophan are aromatic amino acids. The term “hydroxyl-containing amino acid” means that the amino acid R groups contain a hydroxyl group, and are hydrophilic. Serine, Cysteine, Threonine and Methionine are hydroxyl-containing amino acids.

The term “basic amino acid” means that the amino acid R groups are nitrogen containing and are basic at neutral pH. Histidine, Lysine and Arginine are basic amino acids.

The term “cyclic amino acid” means that the amino acid R groups have an aliphatic cyclic structure. Proline is the only cyclic aliphatic amino acid.

The term “acidic amino acid” means that the amino acid R groups are polar and are negatively charged at physiological pH. Aspartate and Glutamate are acidic amino acids.

The term “amide amino acid” means that the amino acid R groups contain an amide group. Asparagine and Glutamine are amide amino acids.

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g., the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

As used herein, the term “biomarker” refers to a gene that is differentially expressed in individuals having a disease of interest, for example, a gene that is differentially expressed in individuals having cancer. In one embodiment, PD-L1 is a biomarker whose expression in tumours may be indicative as to whether or not a patient would respond to a particular type of treatment, in particular, whether a patient would response to treatment targeting PD-L1, for example, immunotherapy using anti-PD-L1 antibodies. In one embodiment, PD-L1 is a biomarker whose expression in tumours may be indicative as to whether or not a patient would respond to a particular type of treatment, in particular, whether a patient would response to treatment targeting PD-1, for example, immunotherapy using anti-PD-1 antibodies. In another embodiment, PD-L1 may be free or membrane bound. In another embodiment, PD-L1 may be fixed or unfixed.

As used herein, a “buffer” refers to a chemical agent that is able to absorb a certain quantity of acid or base without undergoing a strong variation in pH.

As used herein, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

The term “chemotherapeutic agent” or “chemotherapy” refers to a therapeutic agent whose primary purpose is to destroy cancer cells, typically by interfering with the tumour cell's ability to grow or multiply. There are many different types of chemotherapeutic agents, with more than 50 approved chemotherapy drugs available. Chemotherapeutic drugs can be classified based on how they work. Alkylating drugs kill cancer cells by directly attacking DNA, the genetic material of the genes. Cyclophosphamide is an alkylating drug. Antimetabolites interfere with the production of DNA and keep cells from growing and multiplying. An example of an antimetabolite is 5-fluorouracil (5-FU). Anti-tumour antibiotics are made from natural substances such as fungi in the soil. They interfere with important cell functions, including production of DNA and cell proteins. Doxorubicin and bleomycin belong to this group of chemotherapy drugs. Plant alkaloids prevent cells from dividing normally. Vinblastine and vincristine are plant alkaloids obtained from the periwinkle plant. Steroid hormones slow the growth of some cancers that depend on hormones. For example, tamoxifen is used to treat breast cancers that depend on the hormone estrogen for growth. DNA damage response (DDR) inhibitors, such as PARP inhibitors, block DNA repair mechanisms following single or double stranded breaks.

Examples of chemotherapeutic agents include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19th Ed. (Mack Publishing Co. 1995), and in Goodman and Gilman's The Pharmacological Basis of Therapeutics, 7th Ed. (MacMillan Publishing Co. 1985). Another example of chemotherapeutic agents is the class of antibody-conjugated toxins, including, but not limited to pyrrolobenzodiazepines, maytansanoids, calicheamicin, etc. Other suitable toxins and/or chemotherapeutic agents are known to those of skill in the art.

As used herein, the term “composition” is intended to encompass a product containing the specified ingredients (e.g., an antibody of the invention) in, optionally, the specified amounts, as well as any product which results, directly or indirectly, from combination of the specified ingredients in, optionally, the specified amounts.

As used herein the term “comprising” or “comprises” is used in reference to antibodies, fragments, uses, compositions, methods, and respective component(s) thereof, that are essential to the method or composition, yet open to the inclusion of unspecified elements, whether essential or not.

The term “consisting of” refers to antibodies, fragments, uses, compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

In the context of a polypeptide, the term “derivative” as used herein refers to a polypeptide that comprises an amino acid sequence of a hPD-L1 polypeptide, a fragment of a hPD-L1 polypeptide, or an antibody that specifically binds to a hPD-L1 polypeptide which has been altered by the introduction of amino acid residue substitutions, deletions or additions. The term “derivative” as used herein also refers to a hPD-L1 polypeptide, a fragment of a hPD-L1 polypeptide, or an antibody that specifically binds to a hPD-L1 polypeptide which has been chemically modified, e.g., by the covalent attachment of any type of molecule to the polypeptide. For example, but not by way of limitation, a hPD-L1 polypeptide, a fragment of a hPD-L1 polypeptide, or a hPD-L1 antibody may be chemically modified, e.g., by glycosylation, acetylation, pegylation, phosphorylation, amidation, derivatization by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a hPD-L1 polypeptide, a fragment of a hPD-L1 polypeptide, or a hPD-L1 antibody may be chemically modified by chemical modifications using techniques known to those of skill in the art, including, but not limited to specific chemical cleavage, acetylation, formulation, metabolic synthesis of tunicamycin, etc. Further, a derivative of a hPD-L1 polypeptide, a fragment of a hPD-L1 polypeptide, or a hPD-L1 antibody may contain one or more non-classical amino acids. A polypeptide derivative possesses a similar or identical function as a hPD-L1 polypeptide, a fragment of a hPD-L1 polypeptide, or a hPD-L1 antibody described herein.

The term “effector function” as used herein is meant to refer to one or more of antibody dependant cell mediated cytotoxic activity (ADCC), complement-dependant cytotoxic activity (CDC) mediated responses, Fc-mediated phagocytosis or antibody dependant cellular phagocytosis (ADCP) and antibody recycling via the FcRn receptor.

An “effective amount” refers to an amount effective, at dosages and for periods of time necessary, to achieve the desired effect, including a therapeutic or prophylactic result. A “therapeutically effective amount” refers to the minimum concentration required to effect a measurable improvement or prevention of a particular disorder. A therapeutically effective amount herein may vary according to factors such as the disease state, age, sex, and weight of the patient, and the ability of the antibody to elicit a desired response in the individual. A therapeutically effective amount is also one in which toxic or detrimental effects of the antibody are outweighed by the therapeutically beneficial effects. A “prophylactically effective amount” refers to an amount effective, at the dosages and for periods of time necessary, to achieve the desired prophylactic result. In some embodiments, the effective amount of an antibody of the invention is from about 0.1 mg/kg (mg of antibody per kg weight of the subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some embodiments, “effective amount” as used herein also refers to the amount of an antibody of the invention to achieve a specified result (e.g., inhibition of a hPD-L1 biological activity of a cell).

The term “epitope” as used herein refers to a localized region on the surface of an antigen, such as hPD-L1 polypeptide or hPD-L1 polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a hPD-L1 epitope is a three-dimensional surface feature of a hPD-L1 polypeptide (e.g., in a trimeric form of a hPD-L1 polypeptide). In other embodiments, a hPD-L1 epitope is linear feature of a hPD-L1 polypeptide (e.g., in a trimeric form or monomeric form of the hPD-L1 polypeptide). Antibodies provided herein may specifically bind to an epitope of the monomeric (denatured) form of hPD-L1, an epitope of the trimeric (native) form of hPD-L1, or both the monomeric (denatured) form and the trimeric (native) form of hPD-L1. In specific embodiments, the antibodies provided herein specifically bind to an epitope of the trimeric form of hPD-L1 but do not specifically bind the monomeric form of hPD-L1.

The term “excipients” as used herein refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

As used herein, the term “fixed” or “fixation” refers to a chemical process by which biological tissues are preserved from decay, to prevent autolysis or putrefaction. In general, fixation involves exposing the tissue to chemical compounds such as alcohols or aldehydes such as formaldehyde to terminate ongoing biochemical reactions. In some instances, fixation may also increase the mechanical strength or stability of the treated tissues. The term “unfixed” refers to a tissue that has not been subjected to a chemical process to prevent tissue decay. As used herein, the term “surface expressed” means that the protein is embedded in or spans a cell membrane or is associated with a protein that is embedded in or spans a cell membrane (i.e., a membrane associated protein). In one embodiment, a surface expressed protein includes one or more transmembrane domains. In another embodiment, the protein is associated with the exterior or interior surface of a cell membrane indirectly via association with another membrane spanning protein (i.e., the surface expressed protein is not spanning the cell membrane itself). In general, surface expressed proteins that are integrated into a cell membrane or expressed endogenously within a cell are more likely to fold in the correct conformation than recombinantly produced free forms of the same protein. In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, PD-L1 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a hPD-L1 polypeptide or an antibody that specifically binds to a hPD-L1 polypeptide. In a specific embodiment, a fragment of a hPD-L1 polypeptide or an antibody that specifically binds to a hPD-L1 antigen retains at least 1, at least 2, or at least 3 functions of the polypeptide or antibody.

The term “free” refers to a polypeptide, for example, PD-L1 or fragments and variants thereof, that is combined with a buffer, wherein the polypeptide is not associated with a cell surface or cell membrane. As such, the term “free” can refer to a polypeptide that is capable of surface expression (i.e., includes one or more transmembrane domains or membrane association domains), but that is not, in its present state, expressed on the surface of a cell or bound to a protein that is expressed on the surface of a cell. A free polypeptide can also refer to a free recombinant or native or unbound polypeptide. In the context of phage display, a free antigen can be selected in solution (referred to herein as a “soluble selection”) or adsorbed to a surface, for example, adsorbed to the surface of a 96 well plate (referred to herein as “biopanning selection”).

The term “fusion protein” as used herein refers to a polypeptide that comprises an amino acid sequence of an antibody and an amino acid sequence of a heterologous polypeptide or protein (i.e., a polypeptide or protein not normally a part of the antibody (e.g., a non-anti-hPD-L1 antigen antibody)). The term “fusion” when used in relation to hPD-L1 or to an anti-hPD-L1 antibody refers to the joining of a peptide or polypeptide, or fragment, variant and/or derivative thereof, with a heterologous peptide or polypeptide. Preferably, the fusion protein retains the biological activity of the hPD-L1 or anti-hPD-L1 antibody. In certain embodiments, the fusion protein comprises a hPD-L1 antibody VH domain, VL domain, VH CDR (one, two or three VH CDRs), and/or VL CDR (one, two or three VL CDRs), wherein the fusion protein specifically binds to a hPD-L1 epitope.

The term “heavy chain” when used with reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG1, IgG3 and IgG4. Preferably the heavy chain is a human heavy chain. In the human population, multiple heavy chain constant region alleles, of each immunoglobulin or immunoglobulin subclass, exist. The nucleotide and amino acid sequences of these allelic variants are accessible on publicly available databases such as IMGT, ENSEMBL Swiss-Prot and Uniprot. Allelic variants may also be identified in various genome sequencing projects. In one embodiment, the antibodies and antibody fragments disclosed herein comprise a heavy chain encoded by a IgG1 constant region allele, which includes, but is not limited to, human IGHG1*01 (Seq ID Nos: 340, 341 & 537), IGHG1*02 (Seq ID Nos: 340, & 341 & 537), IGHG1*03 (Seq ID Nos: 523 & 524), IGHG1*04 (Seq ID Nos: 525 & 526) and IGHG1*05 (Seq ID Nos: 340, 341 & 537). In one embodiment, the antibodies and antibody fragments disclosed herein comprise a protein encoded by a IgG2 constant region allele, which includes, but is not limited to, human IGHG2*01 (Seq ID Nos: 527 & 528), IGHG2*02 (Seq ID Nos: 529 & 530), IGHG2*03 (Seq ID Nos: 527 & 528), IGHG2*04 (Seq ID Nos: 531 & 532), IGHG2*05 (Seq ID Nos: 527 & 528) and IGHG2*06 (Seq ID Nos: 533 & 534). In one embodiment, the antibodies or antibody fragments disclosed herein comprise a protein encoded by an IgG3 constant region allele, which includes but is not limited to human IGHG3*01, IGHG3*02, IGHG3*03, IGHG3*04, IGHG3*05, IGHG3*06, IGHG3*07, IGHG3*08, IGHG3*09, IGHG3*10, IGHG3*11, IGHG3*12, IGHG3*13, IGHG3*14, IGHG3*15, IGHG3*16, IGHG3*17, IGHG3*18 and IGHG3*19. In one embodiment, the antibodies or antibody fragments disclosed herein comprise a protein encoded by a IgG4 constant region allele, which includes but is not limited to human IGHG4*01 (Seq ID Nos: 192 & 193), IGHG4*02 (Seq ID Nos: 194 & 195), IGHG4*03 (Seq ID Nos: 196 & 197) and IGHG4*04 (Seq ID Nos: 192 & 193). In another example, the heavy chain is a disabled IgG isotype, e.g. a disabled IgG4. In certain embodiments, the antibodies of the invention comprise a human gamma 4 constant region. In another embodiment, the heavy chain constant region does not bind Fc-γ receptors, and e.g. comprises a Leu235Glu mutation. In another embodiment, the heavy chain constant region comprises a Ser228Pro mutation to increase stability. In another embodiment, the heavy chain constant region is IgG4-PE (SEQ ID NO: 199). In In another embodiment, the antibodies and antibody fragments disclosed herein comprise a heavy chain constant region encoded by a murine IgG1 constant region allele, which includes but is not limited to mouse IGHG1*01 or IGHG1*02. In one embodiment, the antibodies and antibody fragments disclosed herein comprise a heavy chain constant region encoded by a murine IgG2 constant region allele, which includes, but is not limited to, mouse IGHG2A*01, IGHG2A*02, IGHG2B*01, IGHG2B*02, IGHG2C*01, IGHG2C*02 or IGHG2C*03. In one embodiment, the antibodies or antibody fragments disclosed herein comprise a protein encoded by a murine IgG3 constant region allele, which includes but is not limited to mouse IGHG3*01.

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “an IL-2 cytokine” as used herein refers to a cytokine-like molecule which has a similar activity to a wild-type IL-2. It may have activity at the high (αβγ) affinity IL-2 receptor and/or the intermediate affinity (αβ) IL-2 receptor. The cytokine may be a variant IL-2 cytokine having one or more amino acid deletions, substitutions or additions.

The term “immunomodulatory agent” and variations thereof including, but not limited to, immunomodulatory agents, as used herein refer to an agent that modulates a host's immune system. In certain embodiments, an immunomodulatory agent is an immunosuppressant agent. In certain other embodiments, an immunomodulatory agent is an immunostimulatory agent. In accordance with the invention, an immunomodulatory agent used in the combination therapies of the invention does not include an anti-hPD-L1 antibody or antigen-binding fragment. Immunomodulatory agents include, but are not limited to, small molecules, peptides, polypeptides, proteins, fusion proteins, antibodies, inorganic molecules, mimetic agents, and organic molecules.

The term “in combination” in the context of the administration of other therapies refers to the use of more than one therapy. The use of the term “in combination” does not restrict the order in which therapies are administered to a subject with a disease. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a hPD-L1-mediated disease. Any additional therapy can be administered in any order with the other additional therapies. In certain embodiments, the antibodies of the invention can be administered in combination with one or more therapies (e.g., therapies that are not the antibodies of the invention that are currently administered to prevent, treat, manage, and/or ameliorate a hPD-L1-mediated disease. Non-limiting examples of therapies that can be administered in combination with an antibody of the invention include analgesic agents, anaesthetic agents, antibiotics, or immunomodulatory agents or any other agent listed in the U.S. Pharmacopoeia and/or Physician's Desk Reference.

The term “immunocytokine”, as used herein refers to an antibody format which is fused to a cytokine molecule. The antibody format may be any of those described herein, and the cytokine may be fused directly, or by means of a linker or chemical conjugation to either the N- or C-terminus of the heavy or the light chain of the antibody format.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some embodiments, an injection device can be an intravenous device or IV device, which is a type of injection device used when the target tissue is the blood within the circulatory system, e.g., the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody or protein is one that has been identified, separated and/or recovered from a component of its production environment (e.g., natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a preferred embodiment, antibodies of the invention are isolated or purified.

The terms “Kabat numbering,” and like terms are recognized in the art and refer to a system of numbering amino acid residues which are more variable (i.e. hypervariable) than other amino acid residues in the heavy chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

“Label” or “labelled” as used herein refers to the addition of a detectable moiety to a polypeptide, for example, a radiolabel, fluorescent label, enzymatic label, chemiluminescent label or a biotinyl group or gold. Radioisotopes or radionuclides may include ³H, ¹⁴C, ¹⁵N, ³⁵S, ⁹⁰Y, ⁹⁹Tc, ¹¹⁵In, ¹²⁵I, ¹³¹I, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes (e.g. cyanine dyes, e.g. Cy5™, Cy5.5™. or Cy7™); additional fluorescent labels or fluorescers include, such as fluorescein and its derivatives, fluorochrome, GFP (GFP for “Green Fluorescent Protein”), other fluorescent proteins (e.g. mCherry, mTomato), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “light chain” when used in reference to an antibody refers to the immunoglobulin light chains, of which there are two types in mammals, lambda (A) and kappa (κ). Preferably, the light chain is a human light chain. Preferably the light chain constant region is a human constant region. In the human population, multiple light chain constant region alleles exist. The nucleotide and amino acid sequences of these allelic variants are accessible on publicly available databases such as IMGT, ENSEMBL, Swiss-Prot and Uniprot. In one embodiment, the antibodies or antibody fragments disclosed herein comprise a protein encoded by a human K constant region allele, which includes, but is not limited to, IGKC*01 (Seq ID Nos: 206 & 207), IGKC*02 (Seq ID Nos: 208 & 209), IGKC*03 (Seq ID Nos: 210 & 211), IGKC*04 (Seq ID Nos: 212 & 213) and IGKC*05 (Seq ID Nos: 214 & 215). In one embodiment, the antibodies or antibody fragments disclosed herein comprise a protein encoded by a human A constant region allele, which includes but is not limited to IGLC1*01 (Seq ID Nos: 216 & 217), IGLC1*02 (Seq ID Nos: 218, 219 & 220), IGLC2*01 (Seq ID Nos: 221, 222 & 538), IGLC2*02 (Seq ID Nos: 224 & 225), IGLC2*03 (Seq ID Nos: 224 & 225), IGLC3*01 (Seq ID Nos: 226 & 227), IGLC3*02 (Seq ID Nos: 228 & 229), IGLC3*03 (Seq ID Nos: 230 & 231), IGLC3*04 (Seq ID Nos: 232 & 233), IGLC6*01 (Seq ID Nos: 234 & 235), IGLC7*01 (Seq ID Nos: 236 & 237), IGLC7*02 (Seq ID Nos: 236 & 237), IGLC7*03 (Seq ID Nos: 535 & 536). In another embodiment, the antibodies and antibody fragments disclosed herein comprise a light chain constant region encoded by a mouse K constant region allele, which includes, but is not limited to, IGKC*01, IGKC*03 or IGKC*03. In another embodiment, the antibodies and antibody fragments disclosed herein comprise a light chain constant region encoded by a mouse A constant region allele, which includes, but is not limited to, IGLC1*01, IGLC2*01 or IGLC3*01.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEG ALIGN™ (DNASTAR) software. In one embodiment, the % homology is about 70%. In one embodiment, the % homology is about 75%. In one embodiment, the % homology is about 80%. In one embodiment, the % homology is about 85%. In one embodiment, the % homology is about 90%. In one embodiment, the % homology is about 92%. In one embodiment, the % homology is about 95%. In one embodiment, the % homology is about 97%. In one embodiment, the % homology is about 98%. In one embodiment, the % homology is about 99%. In one embodiment, the % homology is 100%.

The term “naturally occurring” or “native” when used in connection with biological materials such as nucleic acid molecules, polypeptides, host cells, and the like, refers to those which are found in nature and not manipulated by a human being.

As used herein, “packaging” refers to how the components are organized and/or restrained into a unit fit for distribution and/or use. Packaging can include, e.g., boxes, bags, syringes, ampoules, vials, tubes, clamshell packaging, barriers and/or containers to maintain sterility, labelling, etc.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

As used herein, the terms “prevent,” “preventing,” and “prevention” refer to the total or partial inhibition of the development, recurrence, onset or spread of a hPD-L1-mediated disease and/or symptom related thereto, resulting from the administration of a therapy or combination of therapies provided herein (e.g., a combination of prophylactic or therapeutic agents, such as an antibody of the invention).

The term “soluble” refers to a polypeptide, such as PD-L1 and variants or fragments thereof, that is lacking one or more transmembrane or cytoplasmic domains found in the native or membrane-associated form. In one embodiment, the “soluble” form of PD-L1 lacks both the transmembrane domain and the cytoplasmic domain.

The term “subject” or “patient” refers to any animal, including, but not limited to, mammals. As used herein, the term “mammal” refers to any vertebrate animal that suckle their young and either give birth to living young (eutharian or placental mammals) or are egg-laying (metatharian or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like.

As used herein “substantially all” refers to refers to at least about 60%, at least about 70%, at least about 75%, at least about 80%, at least about 85%, at least about 90%, at least about 95%, at least about 98%, at least about 99%, or about 100%.

The term “substantially free of surfactant” as used herein refers to a formulation of an antibody that specifically binds to a hPD-L1 antigen, said formulation containing less than 0.0005%, less than 0.0003%, or less than 0.0001% of surfactants and/or less than 0.0005%, less than 0.0003%, or less than 0.0001% of surfactants.

The term “substantially free of salt” as used herein refers to a formulation of an antibody that specifically binds to a hPD-L1 antigen, said formulation containing less than 0.0005%, less than 0.0003%, or less than 0.0001% of inorganic salts.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and non-ionic surfactants. Surfactants are often used as wetting, emulsifying, solubilizing, and dispersing agents for various pharmaceutical compositions and preparations of biological materials.

As used herein, the term “tag” refers to any type of moiety that is attached to, e.g., a polypeptide and/or a polynucleotide that encodes a hPD-L1 or hPD-L1 antibody or antigen binding fragment thereof. For example, a polynucleotide that encodes a hPD-L1, hPD-L1 antibody or antigen binding fragment thereof can contain one or more additional tag-encoding nucleotide sequences that encode a, e.g., a detectable moiety or a moiety that aids in affinity purification. When translated, the tag and the antibody can be in the form of a fusion protein. The term “detectable” or “detection” with reference to a tag refers to any tag that is capable of being visualized or wherein the presence of the tag is otherwise able to be determined and/or measured (e.g., by quantitation). A non-limiting example of a detectable tag is a fluorescent tag.

As used herein, the term “therapeutic agent” refers to any agent that can be used in the treatment, management or amelioration of a hPD-L1-mediated disease and/or a symptom related thereto. In certain embodiments, the term “therapeutic agent” refers to an antibody of the invention. In certain other embodiments, the term “therapeutic agent” refers to an agent other than an antibody of the invention. Preferably, a therapeutic agent is an agent which is known to be useful for, or has been or is currently being used for the treatment, management or amelioration of a hPD-L1-mediated disease or one or more symptoms related thereto. In specific embodiments, the therapeutic agent is a fully human anti-hPD-L1 antibody, such as a fully human anti-hPD-L1 monoclonal antibody.

As used herein, the term “therapy” refers to any protocol, method and/or agent that can be used in the prevention, management, treatment and/or amelioration of a hPD-L1-mediated disease (e.g. cancer). In certain embodiments, the terms “therapies” and “therapy” refer to a biological therapy, supportive therapy, and/or other therapies useful in the prevention, management, treatment and/or amelioration of a hPD-L1-mediated disease known to one of skill in the art such as medical personnel.

The terms “treat,” “treatment” and “treating” refer to the reduction or amelioration of the progression, severity, and/or duration of a hPD-L1-mediated disease (e.g., cancer) resulting from the administration of one or more therapies (including, but not limited to, the administration of one or more prophylactic or therapeutic agents, such as an antibody of the invention). In specific embodiments, such terms refer to the reduction or inhibition of the binding of hPD-L1 to PD-1, the reduction or inhibition of the binding of hPD-L1 to CD80, and/or the inhibition or reduction of one or more symptoms associated with a hPD-L1-mediated disease, such as cancer. In specific embodiments, such terms refer to the reduction or inhibition of the binding of hPD-L1 to PD-1 and/or CD80, and/or the inhibition or reduction of one or more symptoms associated with a hPD-L1-mediated disease, such as cancer. In an example, the cell is a human cell. In specific embodiments, a prophylactic agent is a fully human anti-hPD-L1 antibody, such as a fully human anti-hPD-L1 monoclonal antibody.

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs of the PD-L1 and heavy chains are primarily responsible for the interaction of the antibody with antigen. Numbering of amino acid positions used herein for PD-L1 antibody sequences, unless otherwise specified, is according to the EU Index, as in Kabat et al. (1991) Sequences of proteins of immunological interest. (U.S. Department of Health and Human Services, Washington, D.C.) 5th ed. (“Kabat et al.”). In preferred embodiments, the variable region is a human variable region.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10:0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

### Antibodies to PD-L1

The antigen-binding site of any anti-PD-L1 antibody may be used in a multispecific antibody according to the present invention. Numerous examples of anti-PD-L1 antibodies are disclosed herein and others are known in the art. Characterisation data for many of the anti-PD-L1 antibodies mentioned here has been published in U.S. Pat. Nos. 9,567,399 and 9,617,338, both incorporated by reference herein.

1D05 has a heavy chain variable region (V_{H}) amino acid sequence of Seq ID No:33, comprising the CDRH1 amino acid sequence of Seq ID No:27 (IMGT) or Seq ID No:30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 has a light chain variable region (V_{L}) amino acid sequence of Seq ID No:43, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No:340, Seq ID No:524, Seq ID No: 526, Seq ID No:528, Seq ID No: 530, Seq ID No: 532 or Seq ID No: 534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No:46).

84G09 has a heavy chain variable (V_{H}) region amino acid sequence of Seq ID No:13, comprising the CDRH1 amino acid sequence of Seq ID No:7 (IMGT) or Seq ID No:10 (Kabat), the CDRH2 amino acid sequence of Seq ID No:8 (IMGT) or Seq ID No:11 (Kabat), and the CDRH3 amino acid sequence of Seq ID No:9 (IMGT) or Seq ID No:12 (Kabat). The heavy chain nucleic acid sequence of the V_{H} domain is Seq ID No:14. 84G09 has a light chain variable region (V_{L}) amino acid sequence of Seq ID No:23, comprising the CDRL1 amino acid sequence of Seq ID No:17 (IMGT) or Seq ID No:20 (Kabat), the CDRL2 amino acid sequence of Seq ID No:18 (IMGT) or Seq ID No:21 (Kabat), and the CDRL3 amino acid sequence of Seq ID No:19 (IMGT) or Seq ID No:22 (Kabat). The light chain nucleic acid sequence of the V_{L} domain is Seq ID No:24. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:15 (heavy chain nucleic acid sequence Seq ID No:16). A full length light chain amino acid sequence is Seq ID No:25 (light chain nucleic acid sequence Seq ID No: 26).

1D05 HC mutant 1 has a heavy chain variable (V_{H}) region amino acid sequence of Seq ID No:47, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 1 has a light chain variable region (V_{L}) amino acid sequence of Seq ID No:43, comprising the CDRL1 amino acid sequence of Seq ID No:37 (IMGT) or Seq ID No: 40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 2 has a heavy chain variable (V_{H}) region amino acid sequence of Seq ID No:48, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 2 has a light chain variable region (V_{L}) amino acid sequence of Seq ID No:43, comprising the CDRL1 amino acid sequence of Seq ID No:37 (IMGT) or Seq ID No: 40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 3 has a heavy chain variable (V_{H}) region amino acid sequence of Seq ID No:49, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 3 has a light chain variable region (V_{L}) amino acid sequence of Seq ID No:43, comprising the CDRL1 amino acid sequence of Seq ID No:37 (IMGT) or Seq ID No: 40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 HC mutant 4 has a heavy chain variable (V_{H}) region amino acid sequence of Seq ID No:342, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). 1D05 HC mutant 4 has a light chain variable region (V_{L}) amino acid sequence of Seq ID No:43, comprising the CDRL1 amino acid sequence of Seq ID No:37 (IMGT) or Seq ID No: 40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No:197, Seq ID No: 199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length light chain amino acid sequence is Seq ID No:45 (light chain nucleic acid sequence Seq ID No:46).

1D05 LC mutant 1 has a heavy chain variable (V_{H}) region amino acid sequence of Seq ID No:33, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 1 has a light chain variable region (V_{L}) amino acid sequence of Seq ID No:50, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 1 is as defined by the Kabat or IMGT systems from the V_{L} sequence of Seq ID No:50. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No: 532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 2 has a heavy chain variable (V_{H}) region amino acid sequence of Seq ID No:33, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 2 has a light chain variable region (V_{L}) amino acid sequence of Seq ID No:51, comprising the CDRL1 amino acid sequence of Seq ID No:37 (IMGT) or Seq ID No:40 (Kabat), the CDRL2 amino acid sequence of Seq ID No:38 (IMGT) or Seq ID No:41 (Kabat), and the CDRL3 amino acid sequence of Seq ID No:39 (IMGT) or Seq ID No:42 (Kabat). The V_{H} domain may be combined with any of the heavy chain constant region sequences described herein, e.g. Seq ID No:193, Seq ID No:195, Seq ID No:197, Seq ID No:199, Seq ID No: 201, Seq ID No:203, Seq ID No:205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36).

1D05 LC mutant 3 has a heavy chain variable (V_{H}) region amino acid sequence of Seq ID No:33, comprising the CDRH1 amino acid sequence of Seq ID No:27 (IMGT) or Seq ID No: 30 (Kabat), the CDRH2 amino acid sequence of Seq ID No:28 (IMGT) or Seq ID No:31 (Kabat), and the CDRH3 amino acid sequence of Seq ID No:29 (IMGT) or Seq ID No:32 (Kabat). The heavy chain nucleic acid sequence of the V_{H} domain is Seq ID No:34. 1D05 LC mutant 3 has a light chain variable region (V_{L}) amino acid sequence of Seq ID No:298, comprising the CDRL1 amino acid sequence of Seq ID No:37 (IMGT) or Seq ID No:40 (Kabat), and the CDRL3 amino acid sequence of Seq ID No:39 (IMGT) or Seq ID No:42 (Kabat). The CDRL2 sequence of 1D05 LC Mutant 3 is as defined by the Kabat or IMGT systems from the V_{L} sequence of Seq ID No:298. The light chain nucleic acid sequence of the V_{L} domain is Seq ID No:44. The V_{H} domain may be combined with any of the heavy chain constant region sequences described herein, e.g. Seq ID No:193, Seq ID No:195, Seq ID No: 197, Seq ID No:199, Seq ID No:201, Seq ID No:203, Seq ID No:205 or Seq ID No:340, Seq ID No:524, Seq ID No:526, Seq ID No:528, Seq ID No:530, Seq ID No:532 or Seq ID No: 534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:35 (heavy chain nucleic acid sequence Seq ID No:36). A full length light chain amino acid sequence is Seq ID No:45 (light chain nucleic acid sequence Seq ID No: 46).

411B08 has a heavy chain variable (V_{H}) region amino acid sequence of Seq ID No: 58, comprising the CDRH1 amino acid sequence of Seq ID No:52 (IMGT) or Seq ID No: 55 (Kabat), the CDRH2 amino acid sequence of Seq ID No:53 (IMGT) or Seq ID No:56 (Kabat), and the CDRH3 amino acid sequence of Seq ID No:54 (IMGT) or Seq ID No:57 (Kabat). The heavy chain nucleic acid sequence of the V_{H} domain is Seq ID No:59. 411B08 has a light chain variable region (V_{L}) amino acid sequence of Seq ID No:68, comprising the CDRL1 amino acid sequence of Seq ID No:62 (IMGT) or Seq ID No:65 (Kabat), the CDRL2 amino acid sequence of Seq ID No:63 (IMGT) or Seq ID No:66 (Kabat), and the CDRL3 amino acid sequence of Seq ID No:64 (IMGT) or Seq ID No:67 (Kabat). The light chain nucleic acid sequence of the V_{L} domain is Seq ID No:69. The V_{H} domain may be combined with any of the heavy chain constant region sequences described herein, e.g. Seq ID No: 193, Seq ID No:195, Seq ID No: 197, Seq ID No: 199, Seq ID No:201, Seq ID No:203, Seq ID No: 205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:60 (heavy chain nucleic acid sequence Seq ID No: 61). A full length light chain amino acid sequence is Seq ID No:70 (light chain nucleic acid sequence Seq ID No:71).

411C04 has a heavy chain variable (V_{H}) region amino acid sequence of Seq ID No: 78, comprising the CDRH1 amino acid sequence of Seq ID No:72 (IMGT) or Seq ID No: 75 (Kabat), the CDRH2 amino acid sequence of Seq ID No:73 (IMGT) or Seq ID No:76 (Kabat), and the CDRH3 amino acid sequence of Seq ID No:74 (IMGT) or Seq ID No:77 (Kabat). The heavy chain nucleic acid sequence of the V_{H} domain is Seq ID No:79. 411C04 has a light chain variable region (V_{L}) amino acid sequence of Seq ID No:88, comprising the CDRL1 amino acid sequence of Seq ID No:82 (IMGT) or Seq ID No:85 (Kabat), the CDRL2 amino acid sequence of Seq ID No:83 (IMGT) or Seq ID No:86 (Kabat), and the CDRL3 amino acid sequence of Seq ID No:84 (IMGT) or Seq ID No:87 (Kabat). The light chain nucleic acid sequence of the V_{L} domain is Seq ID No:89. The V_{H} domain may be combined with any of the heavy chain constant region sequences described herein, e.g. Seq ID No: 193, Seq ID No:195, Seq ID No: 197, Seq ID No: 199, Seq ID No:201, Seq ID No:203, Seq ID No: 205, Seq ID No:340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:80 (heavy chain nucleic acid sequence Seq ID No: 81). A full length light chain amino acid sequence is Seq ID No:90 (light chain nucleic acid sequence Seq ID No:91).

411D07 has a heavy chain variable (V_{H}) region amino acid sequence of Seq ID No: 98, comprising the CDRH1 amino acid sequence of Seq ID No:92 (IMGT) or Seq ID No: 95 (Kabat), the CDRH2 amino acid sequence of Seq ID No:93 (IMGT) or Seq ID No:96 (Kabat), and the CDRH3 amino acid sequence of Seq ID No:94 (IMGT) or Seq ID No:97 (Kabat). The heavy chain nucleic acid sequence of the V_{H} domain is Seq ID No:99. 411D07 has a light chain variable region (V_{L}) amino acid sequence of Seq ID No:108, comprising the CDRL1 amino acid sequence of Seq ID No:102 (IMGT) or Seq ID No:105 (Kabat), the CDRL2 amino acid sequence of Seq ID No:103 (IMGT) or Seq ID No:106 (Kabat), and the CDRL3 amino acid sequence of Seq ID No:104 (IMGT) or Seq ID No:107 (Kabat). The light chain nucleic acid sequence of the V_{L} domain is Seq ID No:109. The V_{H} domain may be combined with any of the heavy chain constant region sequences described herein, e.g. Seq ID No: 193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No: 530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:100 (heavy chain nucleic acid sequence Seq ID No: 101). A full length light chain amino acid sequence is Seq ID No: 110 (light chain nucleic acid sequence Seq ID No:111).

385F01 has a heavy chain variable (V_{H}) region amino acid sequence of Seq ID No: 118, comprising the CDRH1 amino acid sequence of Seq ID No:112 (IMGT) or Seq ID No: 115 (Kabat), the CDRH2 amino acid sequence of Seq ID No:113 (IMGT) or Seq ID No: 116 (Kabat), and the CDRH3 amino acid sequence of Seq ID No:114 (IMGT) or Seq ID No: 117 (Kabat). The heavy chain nucleic acid sequence of the V_{H} domain is Seq ID No:119. 385F01 has a light chain variable region (V_{L}) amino acid sequence of Seq ID No:128, comprising the CDRL1 amino acid sequence of Seq ID No:122 (IMGT) or Seq ID No:125 (Kabat), the CDRL2 amino acid sequence of Seq ID No:123 (IMGT) or Seq ID No:126 (Kabat), and the CDRL3 amino acid sequence of Seq ID No:124 (IMGT) or Seq ID No:127 (Kabat). The light chain nucleic acid sequence of the V_{L} domain is Seq ID No:129. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:120 (heavy chain nucleic acid sequence Seq ID No:121). A full length light chain amino acid sequence is Seq ID No:130 (light chain nucleic acid sequence Seq ID No:131).

386H03 has a heavy chain variable (V_{H}) region amino acid sequence of Seq ID No: 158, comprising the CDRH1 amino acid sequence of Seq ID No:152 (IMGT) or Seq ID No: 155 (Kabat), the CDRH2 amino acid sequence of Seq ID No:153 (IMGT) or Seq ID No: 156 (Kabat), and the CDRH3 amino acid sequence of Seq ID No:154 (IMGT) or Seq ID No: 157 (Kabat). The heavy chain nucleic acid sequence of the V_{H} domain is Seq ID No:159. 386H03 has a light chain variable region (V_{L}) amino acid sequence of Seq ID No:168, comprising the CDRL1 amino acid sequence of Seq ID No:162 (IMGT) or Seq ID No:165 (Kabat), the CDRL2 amino acid sequence of Seq ID No:163 (IMGT) or Seq ID No:166 (Kabat), and the CDRL3 amino acid sequence of Seq ID No:164 (IMGT) or Seq ID No:167 (Kabat). The light chain nucleic acid sequence of the V_{L} domain is Seq ID No:169. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:160 (heavy chain nucleic acid sequence Seq ID No:161). A full length light chain amino acid sequence is Seq ID No:170 (light chain nucleic acid sequence Seq ID No:171).

389A03 has a heavy chain variable (V_{H}) region amino acid sequence of Seq ID No: 178, comprising the CDRH1 amino acid sequence of Seq ID No:172 (IMGT) or Seq ID No: 175 (Kabat), the CDRH2 amino acid sequence of Seq ID No:173 (IMGT) or Seq ID No: 176 (Kabat), and the CDRH3 amino acid sequence of Seq ID No:174 (IMGT) or Seq ID No: 177 (Kabat). The heavy chain nucleic acid sequence of the V_{H} domain is Seq ID No:179. 389A03 has a light chain variable region (V_{L}) amino acid sequence of Seq ID No:188, comprising the CDRL1 amino acid sequence of Seq ID No:182 (IMGT) or Seq ID No:185 (Kabat), the CDRL2 amino acid sequence of Seq ID No:183 (IMGT) or Seq ID No:186 (Kabat), and the CDRL3 amino acid sequence of Seq ID No:184 (IMGT) or Seq ID No:187 (Kabat). The light chain nucleic acid sequence of the V_{L} domain is Seq ID No:189. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:180 (heavy chain nucleic acid sequence Seq ID No:181). A full length light chain amino acid sequence is Seq ID No:190 (light chain nucleic acid sequence Seq ID No:191).

413D08 has a heavy chain variable (V_{H}) region amino acid sequence of Seq ID No: 138, comprising the CDRH1 amino acid sequence of Seq ID No:132 (IMGT) or Seq ID No: 135 (Kabat), the CDRH2 amino acid sequence of Seq ID No:133 (IMGT) or Seq ID No: 136 (Kabat), and the CDRH3 amino acid sequence of Seq ID No:134 (IMGT) or Seq ID No: 137 (Kabat). The heavy chain nucleic acid sequence of the V_{H} domain is Seq ID No:139. 413D08 has a light chain variable region (V_{L}) amino acid sequence of Seq ID No:148, comprising the CDRL1 amino acid sequence of Seq ID No:142 (IMGT) or Seq ID No:145 (Kabat), the CDRL2 amino acid sequence of Seq ID No:143 (IMGT) or Seq ID No:146 (Kabat), and the CDRL3 amino acid sequence of Seq ID No:144 (IMGT) or Seq ID No:147 (Kabat). The light chain nucleic acid sequence of the V_{L} domain is Seq ID No:149. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No: 140 (heavy chain nucleic acid sequence Seq ID No:141). A full length light chain amino acid sequence is Seq ID No:150 (light chain nucleic acid sequence Seq ID No:151).

413G05 has a heavy chain variable (V_{H}) region amino acid sequence of Seq ID No: 244, comprising the CDRH1 amino acid sequence of Seq ID No:238 (IMGT) or Seq ID No: 241 (Kabat), the CDRH2 amino acid sequence of Seq ID No:239 (IMGT) or Seq ID No: 242 (Kabat), and the CDRH3 amino acid sequence of Seq ID No:240 (IMGT) or Seq ID No: 243 (Kabat). The heavy chain nucleic acid sequence of the V_{H} domain is Seq ID No:245. 413G05 has a light chain variable region (V_{L}) amino acid sequence of Seq ID No:254, comprising the CDRL1 amino acid sequence of Seq ID No:248 (IMGT) or Seq ID No:251 (Kabat), the CDRL2 amino acid sequence of Seq ID No:249 (IMGT) or Seq ID No:252 (Kabat), and the CDRL3 amino acid sequence of Seq ID No:250 (IMGT) or Seq ID No:253 (Kabat). The light chain nucleic acid sequence of the V_{L} domain is Seq ID No:255. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:246 (heavy chain nucleic acid sequence Seq ID No:247). A full length light chain amino acid sequence is Seq ID No:256 (light chain nucleic acid sequence Seq ID No:257).

413F09 has a heavy chain variable (V_{H}) region amino acid sequence of Seq ID No: 264, comprising the CDRH1 amino acid sequence of Seq ID No:258 (IMGT) or Seq ID No: 261 (Kabat), the CDRH2 amino acid sequence of Seq ID No:259 (IMGT) or Seq ID No: 262 (Kabat), and the CDRH3 amino acid sequence of Seq ID No:260 (IMGT) or Seq ID No: 263 (Kabat). The heavy chain nucleic acid sequence of the V_{H} domain is Seq ID No:265. 413F09 has a light chain variable region (V_{L}) amino acid sequence of Seq ID No:274, comprising the CDRL1 amino acid sequence of Seq ID No:268 (IMGT) or Seq ID No:271 (Kabat), the CDRL2 amino acid sequence of Seq ID No:269 (IMGT) or Seq ID No:272 (Kabat), and the CDRL3 amino acid sequence of Seq ID No:270 (IMGT) or Seq ID No:273 (Kabat). The light chain nucleic acid sequence of the V_{L} domain is Seq ID No:275. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:266 (heavy chain nucleic acid sequence Seq ID No:267). A full length light chain amino acid sequence is Seq ID No:276 (light chain nucleic acid sequence Seq ID No:277).

414B06 has a heavy chain variable (V_{H}) region amino acid sequence of Seq ID No: 284, comprising the CDRH1 amino acid sequence of Seq ID No:278 (IMGT) or Seq ID No: 281 (Kabat), the CDRH2 amino acid sequence of Seq ID No:279 (IMGT) or Seq ID No: 282 (Kabat), and the CDRH3 amino acid sequence of Seq ID No:280 (IMGT) or Seq ID No: 283 (Kabat). The heavy chain nucleic acid sequence of the V_{H} domain is Seq ID No:285. 414B06 has a light chain variable region (V_{L}) amino acid sequence of Seq ID No:294, comprising the CDRL1 amino acid sequence of Seq ID No:288 (IMGT) or Seq ID No: 291 (Kabat), the CDRL2 amino acid sequence of Seq ID No:289 (IMGT) or Seq ID No:292 (Kabat), and the CDRL3 amino acid sequence of Seq ID No:290 (IMGT) or Seq ID No:293 (Kabat). The light chain nucleic acid sequence of the V_{L} domain is Seq ID No:295. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:286 (heavy chain nucleic acid sequence Seq ID No:287). A full length light chain amino acid sequence is Seq ID No:296 (light chain nucleic acid sequence Seq ID No:297).

416E01 has a heavy chain variable region (V_{H}) amino acid sequence of Seq ID No: 349, comprising the CDRH1 amino acid sequence of Seq ID No:343 (IMGT) or Seq ID No: 346 (Kabat), the CDRH2 amino acid sequence of Seq ID No:344 (IMGT) or Seq ID No: 347 (Kabat), and the CDRH3 amino acid sequence of Seq ID No:345 (IMGT) or Seq ID No: 348 (Kabat). The heavy chain nucleic acid sequence of the V_{H} domain is Seq ID No:350. 416E01 has a light chain variable region (V_{L}) amino acid sequence of Seq ID No:359, comprising the CDRL1 amino acid sequence of Seq ID No:353 (IMGT) or Seq ID No:356 (Kabat), the CDRL2 amino acid sequence of Seq ID No:354 (IMGT) or Seq ID No:357 (Kabat), and the CDRL3 amino acid sequence of Seq ID No:355 (IMGT) or Seq ID No:358 (Kabat). The light chain nucleic acid sequence of the V_{L} domain is Seq ID No:360. The V_{H} domain may be combined with any of the heavy chain constant region sequences described herein, e.g. Seq ID No:193, Seq ID No:195, Seq ID No:197, Seq ID No:199, Seq ID No:201, Seq ID No:203, Seq ID No:205, Seq ID No:340, Seq ID No:524, Seq ID No:526, Seq ID No: 528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:351 (heavy chain nucleic acid sequence Seq ID No:352). A full length light chain amino acid sequence is Seq ID No:361 (light chain nucleic acid sequence Seq ID No:362).

### Concepts Relating to PD-L1 Antibodies

Concept 1. An antibody or a fragment thereof, which specifically binds to hPD-L1 as defined by Seq ID No:1, and competes for binding to said hPD-L1 with the antibody 1D05, wherein the antibody or fragment comprises a V_{H} domain which comprises a CDRH3 comprising the motif X₁GSGX₂YGX₃X₄FD, wherein X₁, X₂ and X₃ are independently any amino acid, and X₄ is either present or absent, and if present, may be any amino acid (SEQ ID NO: 563).

In these concepts, antibodies or fragments may include or may not include bispecific antibodies. In one embodiment, in these concepts, antibodies or fragments includes bispecific antibodies. In one embodiment, a bispecific antibody does not include a FIT-Ig format. In one embodiment, a bispecific antibody does not include a mAb² format. In one embodiment, a bispecific antibody does not include either a FIT-Ig format or a mAb² format. In one embodiment, the antibody or fragment in these concepts includes a bispecific antibody, but does not include a bispecific antibody having a FIT-Ig format. In one embodiment, the antibody or fragment in these concepts includes a bispecific antibody, but does not include a bispecific antibody having a mAb² format. In one embodiment, the antibody or fragment in these concepts includes a bispecific antibody, but does not include a bispecific antibody having a FIT-Ig format or a mAb² format. In another embodiment, in these concepts, antibodies or fragments include dual binding antibodies.

Preferably, an antibody or a fragment thereof that specifically binds to a hPD-L1 antigen does not cross-react with other antigens (but may optionally cross-react with PD-L1 of a different species, e.g., rhesus, cynomolgus, or murine). An antibody or a fragment thereof that specifically binds to a hPD-L1 antigen can be identified, for example, by immunoassays, BIAcore™, or other techniques known to those of skill in the art. An antibody or a fragment thereof binds specifically to a hPD-L1 antigen when it binds to a hPD-L1 antigen with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times background. See, e.g., Paul, ed., 1989, Fundamental Immunology Second Edition, Raven Press, New York at pages 332-336 for a discussion regarding antibody specificity.

In one embodiment, the antibody or fragment is a human antibody. In one embodiment, the antibody or fragment is a human antibody or fragment. In one embodiment, the antibody or fragment is a fully human antibody or fragment. In one embodiment, the antibody or fragment is a fully human monoclonal antibody or fragment.

There is also provided concept 1a: An antibody or a fragment thereof, that specifically binds to hPD-L1 as defined by Seq ID No:1, and competes for binding to said hPD-L1 with the antibody 411B08, wherein the antibody or fragment comprises a V_{H} domain which comprises a CDRH3 comprising the motif ARX₁RX₂X₃SDX₄X₅D, wherein X₁, X₂, X₃, X₄ and X₅ are independently any amino acid (SEQ ID NO: 564).

There is also provided concept 1b: An antibody or a fragment thereof, that specifically binds to hPD-L1 as defined by Seq ID No:1, and competes for binding to said hPD-L1 with the antibody 411B08, wherein the antibody or fragment comprises a V_{H} domain which comprises a CDRH3 comprising the motif X₁RDGSGSY, wherein X₁ is any amino acid (SEQ ID NO: 565).

As provided in the concepts or aspects herein, an anti-PD-L1 antibody or immunocytokine may bind to PD-L1, e.g. human PD-L1 with a K_{D} of less than 50 nM, less than 40 nM, less than 30 nM as determined by surface plasmon resonance. Another embodiment, anti-PD-L1 antibody or immunocytokine may bind to PD-L1, e.g. human PD-L1 with a K_{D} of less than 20 nM, less than 15 nM, less than 10 nM as determined by surface plasmon resonance. anti-PD-L1 antibody or immunocytokine may bind to PD-L1, e.g. human PD-L1 with a K_{D} of less than 8 nM, less than 5 nM, less than 4 nM, less than 3 nM, less than 2 nM or less than 1 nM as determined by surface plasmon resonance. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

In another embodiment, the K_{D} is within a range of 0.01 to 1 nM, or a range of 0.05 to 2 nM, or a range of 0.05 to 1 nM. The K_{D} may be with regard to hPD-L1, cynoPD-L1 and/or mouse PD-L1.

In another embodiment, the anti-PD-L1 antibodies described herein have a K_{ON} rate (e.g. as measured by SPR, e.g. at 25° C. or at 37° C.) of approximately 0.5 to 10 μM, for example approximately 1 to 8 μM or approximately 1 to 7 μM. In another embodiment, the K_{ON} rate is approximately 1 to 5 μM, e.g. approximately 1 μM, approximately 1.5 μM, approximately 2 μM, approximately 2.5 μM or approximately 3 μM. In another embodiment, the K_{ON} rate is approximately 3.5 μM, approximately 4 μM, approximately 4.5 μM, approximately 5 μM or approximately 5.5 μM.

In another embodiment, the anti-PD-L1 antibodies described herein have a K_{OFF} rate (e.g. as measured by SPR, e.g. at 25° C. or at 37° C.) of approximately 0.01 to 100 mM, for example approximately 0.1 to 50 mM or approximately 0.5 to 50 mM. In another embodiment, the K_{OFF} rate is approximately 0.5 to 10 mM, or approximately 0.5 to 10 mM, e.g. approximately 1 mM, approximately 2 mM, approximately 3 mM, approximately 4 mM or approximately 5 mM. In another embodiment, the K_{OFF} rate is approximately 0.6 mM, approximately 0.7 mM, approximately 0.8 mM or approximately 0.9 mM.

In another embodiment, the anti-PD-L1 antibodies (and immunocytokines) described in the concepts and aspects herein provide improved transient expression levels over other anti-PD-L1 antibodies and immunocytokines. Thus, in one embodiment, the anti-PD-L1 antibody (or immunocytokine) is expressed in a HEK293 cell, e.g. a HEK293T cell, at an expression level of approximately 100 μg/mL, or in a range of approximately 100 to 350 μg/mL. In another embodiment, the expression level is above approximately 350 μg/mL. In another embodiment, the anti-PD-L1 antibody (or immunocytokine) is expressed in a CHO cell, e.g. an Expi-CHO cell, at an expression level of approximately 100 g/mL, or in a range of approximately 100 to 350 μg/mL. In another embodiment, the expression level is above approximately 350 μg/mL.

In another embodiment, the anti-PD-L1 antibody (or immunocytokine) is expressed in a CHO cell, e.g. an Expi-CHO cell or a CHO-E7 EBNA cell, at an expression level of approximately 100 μg/mL, or in a range of approximately 100 to 350 μg/mL. In another embodiment, the expression level is above approximately 350 μg/mL. The antibody described herein as 1D05, formatted as a human IgG1 (Seq ID No:340, at 2 L volume in CHO-E7 EBNA cells has an expression level of approximately 115 μg/mL. The antibody described herein as 416E01, formatted as a human IgG1 (Seq ID No:340), at 2 L volume in CHO-E7 EBNA cells has an expression level of approximately 160 μg/mL. The antibody described herein as 1414B06, formatted as a human IgG1 (Seq ID No:340), at 2 L volume in CHO-E7 EBNA cells has an expression level of approximately 783 μg/mL. The antibody described herein as 413G05, formatted as a human IgG1 (Seq ID No:340), at 2 L volume in CHO-E7 EBNA cells has an expression level of approximately 383 μg/mL.

In any of these expression systems, the expression is carried out of a scale of between approximately 0.5 mL and 3 mL, for example between approximately 0.5 mL and 2 mL. In any of these expression systems, the anti-PD-L1 antibody (or immunocytokine) may be expressed from a pTT5 vector. In any of these expression systems, the anti-PD-L1 antibody (or immunocytokine) may be expressed in conjunction with a lipid transfection reagent, and may optionally be expressed in a CHO cell, e.g. an Expi-CHO cell. In any of these expression systems, the anti-PD-L1 antibody (or immunocytokine) may be expressed in conjunction with a PEI transfection reagent, and may optionally be expressed in a CHO cell, e.g. an CHO-E7 EBNA cell. In any of these expression systems, the anti-PD-L1 antibody (or immunocytokine) may be expressed in conjunction with a helper plasmid (e.g. an AKT helper plasmid), and may optionally be expressed in a CHO cell, e.g. an CHO-E7 EBNA cell.

In any of these expression systems, the expression level is between approximately 100 μg/mL and approximately 1500 μg/mL, for example between approximately 100 μg/mL and approximately 1000 μg/mL, or between approximately 200 μg/mL and approximately 1000 μg/mL, or between approximately 350 μg/mL and approximately 1000 μg/mL. In any of these expression systems, the lower limit of expression may be approximately 100 μg/mL, approximately 200 μg/mL, approximately 300 μg/mL, or approximately 400 μg/mL. In another embodiment, the lower limit of expression may be approximately 500 μg/mL, approximately 600 μg/mL, approximately 700 μg/mL, or approximately 800 μg/mL. In any of these expression systems, the upper limit of expression may be approximately 2000 μg/mL, approximately 1800 μg/mL, approximately 1600 μg/mL, or approximately 1500 μg/mL. In another embodiment, the upper limit of expression may be approximately 1250 g/mL, approximately 1000 μg/mL, approximately 900 μg/mL, or approximately 800 μg/mL.

In another embodiment, the expression system is a Lonza expression system, e.g. Lonza X-Ceed® system. In the Lonza expression system, the expression may be carried out at a scale of approximately 30 mL to 2 L, for example 50 mL to 1 L, or 1 L to 2 L. In the Lonza expression system, the anti-PD-L1 antibody (or immunocytokine) may be expressed in conjunction with electroporation, and optionally without any helper plasmids. In the Lonza expression system, the anti-PD-L1 antibody (or immunocytokine) may be expressed at a level of approximately 1 g/L, or approximately 900 mg/L, or approximately 800 mg/L, or approximately 700 mg/L. In another embodiment, In the Lonza expression system, the anti-PD-L1 antibody (or immunocytokine) may be expressed at a level of approximately 600 mg/L or approximately 500 mg/L or approximately 400 mg/L. In the Lonza expression system, the anti-PD-L1 antibody (or immunocytokine) may be expressed at a level of between approximately 400 mg/L and approximately 2 g/L, for example between approximately 500 mg/L and approximately 1.5 g/L, or between approximately 500 mg/L and approximately 1 g/L. In another embodiment, the expression level is above 1 g/L.

Concept 2. The antibody or fragment according to concept 1, wherein X₁ is a hydroxyl-containing amino acid, optionally T. In one embodiment, the hydroxyl-containing amino acid is Serine. In one embodiment, the hydroxyl-containing amino acid is Cysteine. In one embodiment, the hydroxyl-containing amino acid is Threonine. In one embodiment, the hydroxyl-containing amino acid is Methionine. In one embodiment, the hydroxyl-containing amino acid is Serine or Cysteine. In one embodiment, the hydroxyl-containing amino acid is Serine or Threonine. In one embodiment, the hydroxyl-containing amino acid is Serine or Methionine. In one embodiment, the hydroxyl-containing amino acid is Cysteine or Threonine. In one embodiment, the hydroxyl-containing amino acid is Cysteine or Methionine. In one embodiment, the hydroxyl-containing amino acid is Threonine or Methionine. In one embodiment, the hydroxyl-containing amino acid is selected from serine, cysteine, threonine and methionine.

Concept 2a. The antibody or fragment according to concept 1a, wherein X₁ is an aliphatic amino acid or an amide amino acid. In one embodiment, X₁ is selected from Asparagine (N) and valine (V). In one embodiment, X₁ is valine. In one embodiment, X₁ is asparagine.

Concept 2b. The antibody or fragment according to concept 1b, wherein X₁ is an aliphatic amino acid. In one embodiment, X₁ is selected from alanine (A) or valine (V). In one embodiment, X₁ is valine. In one embodiment, X₁ is alanine.

Concept 3. The antibody or fragment according to concept 1 or concept 2, wherein X₂ is a basic amino acid, optionally K. In one embodiment, the hydroxyl-containing amino acid is Histidine. In one embodiment, the hydroxyl-containing amino acid is Lysine. In one embodiment, the hydroxyl-containing amino acid is Arginine. In one embodiment, the hydroxyl-containing amino acid is Histidine or Lysine. In one embodiment, the hydroxyl-containing amino acid is Histidine or Arginine. In one embodiment, the hydroxyl-containing amino acid is Lysine or Arginine. In one embodiment, the hydroxyl-containing amino acid is selected from Histidine, Lysine and Arginine.

Concept 3a. The antibody or fragment according to concept 1a or concept 2a, wherein X₂ is an aliphatic amino acid or an amide amino acid. In one embodiment, X₂ is selected from leucine (L), isoleucine (I), Valine (V), Asparagine (N) and glutamine (Q). In one embodiment, X₂ is selected from leucine (L), isoleucine (I) and Valine (V). In one embodiment, X₂ is selected from Asparagine (N) and glutamine (Q) In one embodiment, X₂ is selected from leucine (L) and glutamine (Q). In one embodiment, X₂ is leucine (L). In one embodiment, X₂ is glutamine (Q).

Concept 4. The antibody or fragment according to any one of concepts 1 to 3, wherein X₂ is a hydroxyl-containing amino acid, optionally S or T. In one embodiment, the hydroxyl-containing amino acid is Serine. In one embodiment, the hydroxyl-containing amino acid is Cysteine. In one embodiment, the hydroxyl-containing amino acid is Threonine. In one embodiment, the hydroxyl-containing amino acid is Methionine. In one embodiment, the hydroxyl-containing amino acid is Serine or Cysteine. In one embodiment, the hydroxyl-containing amino acid is Serine or Threonine. In one embodiment, the hydroxyl-containing amino acid is Serine or Methionine. In one embodiment, the hydroxyl-containing amino acid is Cysteine or Threonine. In one embodiment, the hydroxyl-containing amino acid is Cysteine or Methionine. In one embodiment, the hydroxyl-containing amino acid is Threonine or Methionine. In one embodiment, the hydroxyl-containing amino acid is selected from serine, cysteine, threonine and methionine.

Concept 4a. The antibody or fragment according to any one of concepts 1a, 2a or 3a, wherein X₃ is an aromatic amino acid. In one embodiment, X₃ is selected from Phenylalanine (F), Tyrosine (Y) and Tryptophan (W). In one embodiment, X₃ is selected from Tyrosine (Y) and Tryptophan (W). In one embodiment, X₃ is Tyrosine (Y). In one embodiment, X₃ is Tryptophan (W).

Concept 5. The antibody or fragment according to any one of concepts 1 to 4, wherein X₃ is an aromatic amino acid, optionally W. In one embodiment, the hydroxyl-containing amino acid is Phenylalanine. In one embodiment, the hydroxyl-containing amino acid is Tyrosine. In one embodiment, the hydroxyl-containing amino acid is Tryptophan. In one embodiment, the hydroxyl-containing amino acid is Phenylalanine or Tyrosine. In one embodiment, the hydroxyl-containing amino acid is Phenylalanine or Tryptophan. In one embodiment, the hydroxyl-containing amino acid is Tyrosine or Tryptophan. In one embodiment, the hydroxyl-containing amino acid is selected from Phenylalanine, Tyrosine and Tryptophan.

Concept 5a. The antibody or fragment according to any one of concepts 1a, 2a, 3a or 4a wherein X₄ is an aromatic amino acid. In one embodiment, X₄ is selected from Phenylalanine (F), Tyrosine (Y) and Tryptophan (W). In one embodiment, X₄ is selected from Tyrosine (Y) and Phenylalanine (F). In one embodiment, X₄ is Tyrosine (Y). In one embodiment, X₄ is Phenylalanine (F).

Concept 6. The antibody or fragment according to any one of concepts 1 to 5, wherein X₄ is absent.

Concept 6a. The antibody or fragment according to any one of concepts 1a, 2a, 3a, 4a or 5a wherein X₅ is an aliphatic amino acid or an hydroxyl-containing amino acid. In one embodiment, X₅ is selected from leucine (L), isoleucine (I), Valine (V), Serine(S), Cysteine (C) and Threonine (T). In one embodiment, X₅ is selected from leucine (L), isoleucine (I) and Valine (V). In one embodiment, X₅ is selected from Serine(S), Cysteine (C) and Threonine (T). In one embodiment, X₅ is selected from leucine (L) and Serine(S). In one embodiment, X₅ is Serine(S). In one embodiment, X₅ is leucine (L).

Concept 7. The antibody or fragment according to any one of concepts 1 to 5, wherein X₄ is present.

Concept 8. The antibody or fragment according to concept 7, wherein X₄ is an aliphatic amino acid, optionally G. In one embodiment, the hydroxyl-containing amino acid is selected from Glycine, Alanine, Valine, Leucine and Isoleucine. In one embodiment, the hydroxyl-containing amino acid is selected from Glycine and Alanine. In one embodiment, the hydroxyl-containing amino acid is selected from Glycine and Valine. In one embodiment, the hydroxyl-containing amino acid is selected from Glycine and Leucine. In one embodiment, the hydroxyl-containing amino acid is selected from Glycine and Isoleucine. In one embodiment, the hydroxyl-containing amino acid is selected from Alanine and Valine. In one embodiment, the hydroxyl-containing amino acid is selected from Alanine and Leucine. In one embodiment, the hydroxyl-containing amino acid is selected from Alanine and Isoleucine. In one embodiment, the hydroxyl-containing amino acid is selected from Valine and Leucine. In one embodiment, the hydroxyl-containing amino acid is selected from Valine and Isoleucine. In one embodiment, the hydroxyl-containing amino acid is selected from, Leucine and Isoleucine. In one embodiment, the hydroxyl-containing amino acid selected from three of each of Glycine, Alanine, Valine, Leucine and Isoleucine. In one embodiment, the hydroxyl-containing amino acid selected from four of each of Glycine, Alanine, Valine, Leucine and Isoleucine.

Concept 9. An antibody or a fragment thereof, optionally according to any one of concepts 1 to 8, which specifically binds to hPD-L1, and competes for binding to said hPD-L1 with the antibody 1D05, wherein the antibody or fragment comprises a V_{H} domain which comprises the CDRH3 sequence of SEQ ID NO:29 or 32, or the CDRH3 sequence of SEQ ID NO: 29 or 32 comprising 6 or fewer amino acid substitutions.

Concept 9a: An antibody or a fragment thereof, optionally according to any one of concepts 1 to 8, which specifically binds to hPD-L1, and competes for binding to said hPD-L1 with the antibody 84G09, wherein the antibody or fragment comprises a V_{H} domain which comprises the CDRH3 sequence of SEQ ID NO:9 or 12, or the CDRH3 sequence of SEQ ID NO: 9 or 12 comprising 6 or fewer amino acid substitutions.

Concept 9b: An antibody or a fragment thereof, optionally according to any one of concepts 1 to 8, which specifically binds to hPD-L1, and competes for binding to said hPD-L1 with the antibody 411B08, wherein the antibody or fragment comprises a V_{H} domain which comprises the CDRH3 sequence of SEQ ID NO:54 or 57, or the CDRH3 sequence of SEQ ID NO: 54 or 57 comprising 6 or fewer amino acid substitutions.

Concept 9c: An antibody or a fragment thereof, optionally according to any one of concepts 1 to 8, which specifically binds to hPD-L1, and competes for binding to said hPD-L1 with the antibody 411C04, wherein the antibody or fragment comprises a V_{H} domain which comprises the CDRH3 sequence of SEQ ID No:74 or 77, or the CDRH3 sequence of SEQ ID NO: 74 or 77 comprising 6 or fewer amino acid substitutions.

Concept 9d: An antibody or a fragment thereof, optionally according to any one of concepts 1 to 8, which specifically binds to hPD-L1, and competes for binding to said hPD-L1 with the antibody 411D07, wherein the antibody or fragment comprises a V_{H} domain which comprises the CDRH3 sequence of SEQ ID NO:94 or 97, or the CDRH3 sequence of SEQ ID NO: 94 or 97 comprising 3 or fewer amino acid substitutions.

Concept 9e: An antibody or a fragment thereof, optionally according to any one of concepts 1 to 8, which specifically binds to hPD-L1, and competes for binding to said hPD-L1 with the antibody 385F01, wherein the antibody or fragment comprises a V_{H} domain which comprises the CDRH3 sequence of SEQ ID NO:114 or 117, or the CDRH3 sequence of SEQ ID NO: 114 or 117 comprising 6 or fewer amino acid substitutions.

Concept 9f: An antibody or a fragment thereof, optionally according to any one of concepts 1 to 8, which specifically binds to hPD-L1, and competes for binding to said hPD-L1 with the antibody 386H03, wherein the antibody or fragment comprises a V_{H} domain which comprises the CDRH3 sequence of SEQ ID NO:144 or 147, or the CDRH3 sequence of SEQ ID NO: 144 or 147 comprising 3 or fewer amino acid substitutions.

Concept 9g: An antibody or a fragment thereof, optionally according to any one of concepts 1 to 8, which specifically binds to hPD-L1, and competes for binding to said hPD-L1 with the antibody 389A03, wherein the antibody or fragment comprises a V_{H} domain which comprises the CDRH3 sequence of SEQ ID NO:174 or 177, or the CDRH3 sequence of SEQ ID NO: 174 or 177 comprising 6 or fewer amino acid substitutions.

Concept 9h: An antibody or a fragment thereof, optionally according to any one of concepts 1 to 8, which specifically binds to hPD-L1, and competes for binding to said hPD-L1 with the antibody 413D08, wherein the antibody or fragment comprises a V_{H} domain which comprises the CDRH3 sequence of SEQ ID NO:134 or 137, or the CDRH3 sequence of SEQ ID NO: 134 or 137 comprising 5 or fewer amino acid substitutions.

Concept 9i: An antibody or a fragment thereof, optionally according to any one of concepts 1 to 8, which specifically binds to hPD-L1, and competes for binding to said hPD-L1 with the antibody 413G05, wherein the antibody or fragment comprises a V_{H} domain which comprises the CDRH3 sequence of SEQ ID NO:240 or 243, or the CDRH3 sequence of SEQ ID NO: 240 or 243 comprising 6 or fewer amino acid substitutions.

Concept 9j: An antibody or a fragment thereof, optionally according to any one of concepts 1 to 8, which specifically binds to hPD-L1, and competes for binding to said hPD-L1 with the antibody 413F09, wherein the antibody or fragment comprises a V_{H} domain which comprises the CDRH3 sequence of SEQ ID NO:260 or 263, or the CDRH3 sequence of SEQ ID NO: 260 or 263 comprising 6 or fewer amino acid substitutions.

Concept 9k: An antibody or a fragment thereof, optionally according to any one of concepts 1 to 8, which specifically binds to hPD-L1, and competes for binding to said hPD-L1 with the antibody 414B06, wherein the antibody or fragment comprises a V_{H} domain which comprises the CDRH3 sequence of SEQ ID NO:280 or 283, or the CDRH3 sequence of SEQ ID NO: 280 or 283 comprising 6 or fewer amino acid substitutions.

Concept 91: An antibody or a fragment thereof, optionally according to any one of concepts 1 to 8, which specifically binds to hPD-L1, and competes for binding to said hPD-L1 with the antibody 416E01, wherein the antibody or fragment comprises a V_{H} domain which comprises the CDRH3 sequence of SEQ ID No:345 or 348, or the CDRH3 sequence of SEQ ID No: 345 or 348 comprising 6 or fewer amino acid substitutions.

In all of concepts 9, 9a to l, 17, 17a to l, 18, 18a to l, 19, 19a to l, 22, 22a to l, 23, 23a to 1, 24 and 24a to l, in one embodiment, the CDR comprises one amino acid substitution, which may be a conservative amino acid substitution. In all of concepts 9, 9a to l, 17, 17a to l, 18, 18a to l, 19, 19a to 1, 22, 22a to 1, 23, 23a, 24 and 24a to l, in one embodiment, the CDR comprises two amino acid substitutions, which may be conservative amino acid substitutions. In all of concepts 9, 9a to l, 17, 17a to l, 18, 18a to l, 19, 19a to l, 22, 22a, 22b, 22d, 22f, 22g, 24 and 24a to l, in one embodiment, the CDR comprises three amino acid substitutions, which may be conservative amino acid substitutions. In all of concepts 9, 9a to c, 9e, 9g to k, 17, 17a to c, 17e, 17g to l, 19, 19a, 22, 22d, 22f, 22g, 24 and 24a to l, in one embodiment, the CDR comprises four amino acid substitutions, which may be conservative amino acid substitutions. In all of concepts 9, 9a to c, 9e, 9g to l, 17, 17a to c, 17e, 17g to l, 22d, 22f and 22g, in one embodiment, the CDR comprises five amino acid substitutions, which may be conservative amino acid substitutions. In all of concepts 9, 9a to c, 9e, 9g, 9i to l, 17, 17a to c, 17e, 17g and 17i to l, in one embodiment, the CDR comprises six amino acid substitutions, which may be conservative amino acid substitutions. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g. substituting a charged or hydrophobic amino; acid with alanine), or alternatively, in which a naturally-occurring amino acid is substituted with a non-conventional amino acid.

In one embodiment, the conservative amino acid substitutions are as described herein. For example, the substitution may be of Y with F, T with S or K, P with A, E with D or Q, N with D or G, R with K, G with N or A, T with S or K, D with N or E, I with L or V, F with Y, S with T or A, R with K, G with N or A, K with R, A with S, K or P. In another embodiment, the conservative amino acid substitutions may be wherein Y is substituted with F, T with A or S, I with L or V, W with Y, M with L, N with D, G with A, T with A or S, D with N, I with L or V, F with Y or L, S with A or T and A with S, G, T or V.

Concept 10. An antibody or fragment which specifically binds to hPD-L1 and comprises a V_{H} domain comprising a CDRH3 of from 12 to 20 amino acids and which is derived from the recombination of a human V_{H} gene segment, a human D gene segment and a human J_{H} gene segment, wherein the human J_{H} gene segment is IGHJ5 (e.g. IGHJ5*02). In one embodiment, the CDRH3 is from 14 to 17 amino acids and the human J_{H} gene segment is IGHJ5 (e.g. IGHJ5*02).

There is also provided as concept 10a an antibody or fragment which specifically binds to hPD-L1 and comprises a V_{H} domain comprising a CDRH3 of from 8 to 16 amino acids and which is derived from the recombination of a human V_{H} gene segment, a human D gene segment and a human J_{H} gene segment, wherein the human J_{H} gene segment is selected from IGHJ4 (e.g. IGHJ4*02), IGHJ5 (e.g. IGHJ5*02) and IGHJ6 (e.g. IGHJ6*02). In another embodiment, the human J_{H} gene segment is IGHJ6 (e.g. IGHJ6*02). In another embodiment, the CDRH3 is of from 10 to 17 amino acids and the human J_{H} gene segment is IGHJ6 (e.g. IGHJ6*02). In another embodiment, the human J_{H} gene segment is IGHJ4 (e.g. IGHJ4*02). In another embodiment, the CDRH3 is from 7 to 17 amino acids and the human J_{H} gene segment is IGHJ4 (e.g. IGHJ4*02).

Optionally, the antibody of concept 10 or 10a has any of the features of concepts 1 to 9, including the binding affinities, Kon and Koff rates, expression levels, half-life etc.

Concept 11. The antibody or fragment according to concept 10 or 10a, wherein the human V_{H} gene segment is IGHV3 (e.g. IGHV3-9, such as IGHV3-9*01).

There is also provided as concept 11a an antibody or fragment according to concept 10 or 10a, wherein the human V_{H} gene segment is selected from IGHV3 (e.g. IGHV3-9, such as IGHV3-9*01 or e.g. IGHV3-7, such as IGHV3-7*01 or e.g. IGHV3-33, such as IGHV3-33*01 or e.g. IGHV3-11, such as IGHV3-11*01 or e.g. IGHV3-23, such as IGHV3-23*04), or IGHV4 (e.g. IGHV4-4, such as IGHV4-4*02 or e.g. IGHV4-39, such as IGHV4-39*01). In one embodiment, the human V_{H} gene segment is IGHV3 (e.g. IGHV3-7, such as IGHV3-7*01). In one embodiment, the human V_{H} gene segment is IGHV3 (e.g. IGHV3-33, such as IGHV3-33*01). In one embodiment, the human V_{H} gene segment is IGHV3 (e.g. IGHV3-11, such as IGHV3-11*01). In one embodiment, the human V_{H} gene segment is IGHV3 (e.g. IGHV3-23, such as IGHV3-23*04). In one embodiment, the human V_{H} gene segment is IGHV4 (e.g. e.g. IGHV4-4, such as IGHV4-4*02). In one embodiment, the human V_{H} gene segment is IGHV4 (e.g. IGHV4-39, such as IGHV4-39*01).

There is also provided as concept 11b an antibody or fragment according to concept 10, 10a, 11 or 11a, wherein the human D gene segment is selected from IGHD1 (e.g. IGHD1-20, such as IGHD1-20*01), IGHD3 (e.g. IGHD3-10, such as IGHD3-10*01), IGHD4 (e.g. IGHD4-11, such as IGHD4-11*01), IGHD5 (e.g. IGHD5-7, such as IGHD5-18*01), and IGHD6 (e.g. IGHD6-13, such as IGHD6-13*01). In one embodiment, the human D gene segment is IGHD1 (e.g. IGHD1-20, such as IGHD1-20*01). In one embodiment, the human D gene segment is IGHD3 (e.g. IGHD3-10, such as IGHD3-10*01). In one embodiment, the human D gene segment is IGHD4 (e.g. IGHD4-11, such as IGHD4-11*01). In one embodiment, the human D gene segment is IGHD5 (e.g. IGHD5-18, such as IGHD5-19*01). In one embodiment, the human D gene segment is IGHD6 (e.g. IGHD6-13, such as IGHD6-13*01).

In any of concepts 10, 11 and 11a, the V_{H}, D_{H} and J_{H} gene segments are as described in the combinations for the antibodies in Table 5 hereinbelow. In one embodiment, the antibody heavy chain is derived from a combination of IGHV3 (e.g. IGHV3-7 such as IGHV3-7*01), IGHD4 (e.g. IGHD4-11 such as IGHD4-11*01) and IGHJ4 (e.g. IGHJ4*02). In one embodiment, the antibody heavy chain is derived from a combination of IGHV4 (e.g. IGHV4-4 such as IGHV4-4*02), IGHD3 (e.g. IGHD3-10 such as IGHD3-10*01) and IGHJ4 (e.g. IGHJ4*02). In one embodiment, the antibody heavy chain is derived from a combination of IGHV4 (e.g. IGHV4-39 such as IGHV4-39*01), IGHD6 (e.g. IGHD6-13 such as IGHD6-13*01) and IGHJ1 (e.g. IGHJ1*01). In one embodiment, the antibody heavy chain is derived from a combination of IGHV3 (e.g. IGHV3-33 such as IGHV3-33*01), IGHD5 (e.g. IGHD5-18 such as IGHD5-18*01) and IGHJ6 (e.g. IGHJ6*02). In one embodiment, the antibody heavy chain is derived from a combination of IGHV3 (e.g. IGHV3-11 such as IGHV3-11*01), IGHD1 (e.g. IGHD1-20 such as IGHD1-20*01) and IGHJ6 (e.g. IGHJ6*02). In one embodiment, the antibody heavy chain is derived from a combination of IGHV3 (e.g. IGHV3-23 such as IGHV3-23*04), IGHD5 (e.g. IGHD5-18 such as IGHD5-18*01) and IGHJ4 (e.g. IGHJ4*02). In one embodiment, the antibody heavy chain is derived from a combination of IGHV3 (e.g. IGHV3-7 such as IGHV3-7*01), IGHD5 (e.g. IGHD5-24 such as IGHD5-24*01) and IGHJ4 (e.g. IGHJ4*02). In one embodiment, the antibody heavy chain is derived from a combination of IGHV3 (e.g. IGHV3-23 such as IGHV3-23*04), IGHD6 (e.g. IGHD6-13 such as IGHD6-13*01) and IGHJ4 (e.g. IGHJ4*02).

Concept 12. The antibody or fragment according to concept 10, 10a, 11, 11a or 11b, wherein the antibody or fragment comprises a V_{L} domain which is derived from the recombination of a human Vκ gene segment, and a human Jκ gene segment, wherein the human Vκ gene segment is IGκV1D (e.g. IGκV1D-39, such as IGκV1D-39*01).

There is also provided as concept 12a an antibody or fragment according to any of concepts 10, 10a, 11, 11a or 11b, wherein the human Vκ gene segment is selected from IGκV1 (e.g. IGκV1-17, such as IGκV1-17*01 or e.g. IGκV1-9, such as IGκV1-9*d01 or e.g. IGκV1D-12, such as IGκV1D-12*02 or e.g. IGκV1D-39, such as IGκV1D-39*01), and IGκV4 (e.g. IGκV4-1, such as IGκV4-1*01). In one embodiment, the human Vκ gene segment is IGκV1 (e.g. IGκV1-17, such as IGκV1-17*01). In one embodiment, the human V gene segment is IGκV1 (e.g. IGκV1-9, such as IGκV1-9*d01). In one embodiment, the human Vκ gene segment is IGκV1 (e.g. IGκV1D-12, such as IGκV1D-12*02). In one embodiment, the human Vκ gene segment is IGκV1 (e.g. IGκV1D-39, such as IGκV1D-39*01). In one embodiment, the human Vκ gene segment is IGκV1 IGκV4 (e.g. IGκV4-1, such as IGκV4-1*01)

There is also provided as concept 12b an antibody or fragment according to concept 10, 10a, 11 or 11a, wherein the human Jκ gene segment is selected from IGκJ1 (e.g. IGκJ1*01), IGκJ2 (e.g. IGκJ2*04), IGκJ3 (e.g. IGκJ3*01), IGκJ4 (e.g. IGκJ4*01) or IGκJ5 (e.g. IGκJ5*01). In one embodiment, the human Jκ gene segment is IGκJ1 (e.g. IGκJ1*01). In one embodiment, the human Jκ gene segment is IGκJ2 (e.g. IGκJ2*04). In one embodiment, the human Jκ gene segment is IGκJ3 (e.g. IGκJ3*01). In one embodiment, the human Jκ gene segment is IGκJ4 (e.g. IGκJ4*01). In one embodiment, the human Jκ gene segment is IGκJ5 (e.g. IGκJ5*01).

In any of concepts 12 and 12a, the Vκ and Jκ gene segments are as described in the combinations for the antibodies in Table 5 hereinbelow. In one embodiment, the antibody light chain is derived from a combination of IGκV1D (e.g. IGκV1D-12 such as IGκV1D-12*02) and IGκJ3 (e.g. IGκJ3*01). In one embodiment, the antibody light chain is derived from a combination of IGκV4 (e.g. IGκV4-1 such as IGκV14-1*01) and IGκJ2 (e.g. IGκJ2*04). In one embodiment, the antibody light chain is derived from a combination of IGκV1 (e.g. IGκV1-17 such as IGκV1-17*01) and IGκJ1 (e.g. IGκJ1*01). In one embodiment, the antibody light chain is derived from a combination of IGκV1D (e.g. IGκV1D-12 such as IGκV1D-12*02) and IGκJ4 (e.g. IGκJ4*01). In one embodiment, the antibody light chain is derived from a combination of IGκV1 (e.g. IGκV1-9 such as IGκV1-9*d01) and IGκJ5 (e.g. IGκJ5*01). In one embodiment, the antibody light chain is derived from a combination of IGκV1D (e.g. IGκV1D-12 such as IGκV1D-12*02) and IGκJ5 (e.g. IGκJ5*01).

Concept 13. An antibody or fragment thereof which specifically binds to an epitope that is identical to an epitope to which the antibody 1D05 specifically binds.

Concept 13a. An antibody or fragment thereof which specifically binds to an epitope that is identical to an epitope to which the antibody 84G09 specifically binds.

Concept 13b. An antibody or fragment thereof which specifically binds to an epitope that is identical to an epitope to which the antibody 411B08 specifically binds.

Concept 13c. An antibody or fragment thereof which specifically binds to an epitope that is identical to an epitope to which the antibody 411C04 specifically binds.

Concept 13d. An antibody or fragment thereof which specifically binds to an epitope that is identical to an epitope to which the antibody 411D07 specifically binds.

Concept 13e. An antibody or fragment thereof which specifically binds to an epitope that is identical to an epitope to which the antibody 385F01 specifically binds.

Concept 13f. An antibody or fragment thereof which specifically binds to an epitope that is identical to an epitope to which the antibody 386H03 specifically binds.

Concept 13g. An antibody or fragment thereof which specifically binds to an epitope that is identical to an epitope to which the antibody 389A03 specifically binds.

Concept 13h. An antibody or fragment thereof which specifically binds to an epitope that is identical to an epitope to which the antibody 413D08 specifically binds.

Concept 13i. An antibody or fragment thereof which specifically binds to an epitope that is identical to an epitope to which the antibody 413G05 specifically binds.

Concept 13j. An antibody or fragment thereof which specifically binds to an epitope that is identical to an epitope to which the antibody 413F09 specifically binds.

Concept 13k. An antibody or fragment thereof which specifically binds to an epitope that is identical to an epitope to which the antibody 414B06 specifically binds.

Concept 13l. An antibody or fragment thereof which specifically binds to an epitope that is identical to an epitope to which the antibody 416E01 specifically binds.

The antibodies described in these concepts have the sequences as described hereinabove.

In one embodiment, there is provided an antibody which specifically binds to an epitope which is substantially similar to an epitope to which any of the antibodies in concept 13, 13 a to 13l bind.

Contact amino acid residues involved in the interaction of antibody and antigen may be determined by various known methods to those skilled in the art. In one embodiment, sequential replacement of the amino acids of the antigen sequence (using standard molecular biology techniques to mutate the DNA of the coding sequence of the antigen), in this case hPD-L1 with Alanine (a.k.a Alanine scan), or another unrelated amino acid, may provide residues whose mutation would reduce or ablate the ability of the antibody to recognise the antigen in question. Binding may be assessed using standard techniques, such as, but not limited to, SPR, HTRF, ELISA (which are described elsewhere herein). Other substitutions could be made to enhance the disruption of binding such as changing the charge on the side chain of antigen sequence amino acids (e.g. Lysine change to glutamic acid), switching polar and non-polar residues (e.g. Serine change to leucine). The alanine scan or other amino substitution method may be carried out either with recombinant soluble antigen, or where the target is a cell membrane target, directly on cells using transient or stable expression of the mutated versions. In one embodiment, protein crystallography may be used to determine contact residues between antibody and antigen (i.e. to determine the epitope to which the antibody binds), crystallography allows the direct visualisation of contact residues involved in the antibody-antigen interaction. As well as standard X-ray crystallography, cryo-electro microscopy has been used to determine contact residues between antibodies and HIV capsid protein (see Lee, Jeong Hyun, et al. “Antibodies to a conformational epitope on gp41 neutralize HIV-1 by destabilizing the Env spike.”, Nature communications, 6, (2015)). In one embodiment, if the antibody recognises a linear epitope, short peptides based on the antigen sequence can be produced and binding of the antibody to these peptides can be assessed using standard techniques, such as, but not limited to, SPR, HTRF, ELISA (which are described elsewhere herein). Further investigation of the epitope could be provided by performing an Alanine scan on any peptides that show binding. Alternative to linear peptides, conformational scans could be carried out using Pepscan technology (http://www.pepscan.com/) using their chemical linkage of peptides onto scaffolds, which has been used to determine discontinuous epitopes on CD20 targeting antibodies (Niederfellner, Gerhard, et al. “Epitope characterization and crystal structure of GA101 provide insights into the molecular basis for type I/II distinction of CD20 antibodies.”, Blood, 118.2, (2011), 358-367.). In one embodiment, limited proteolytic digestion and mass spectrophotometry can be used to identify binding epitopes. The antibody-antigen complex is digested by a protease, such as, but not limited to, trypsin. The digested complex peptides are compared to antibody-alone and antigen-alone digestion mass spectrophotometry to determine if a particular epitope is protected by the complexation. Further work involving amino acid substitution, competition binding, may then be employed to narrow down to individual amino acid residues involved in the interaction (see, for example, Suckau, Detlev, et al. “Molecular epitope identification by limited proteolysis of an immobilized antigen-antibody complex and mass spectrometric peptide mapping.”, Proceedings of the National Academy of Sciences, 87.24, (1990), 9848-9852). Thus, in one embodiment, the contact residues of the epitope are identified with an unrelated amino acid scan (e.g. alanine scan). In another embodiment, an unrelated amino acid scan (e.g. alanine scan) is carried out using a technique selected from SPR, HTRF, ELISA, X-ray crystallography, cryo-electro microscopy and a combination of limited proteolytic digestion and mass spectrometry. In one embodiment, the unrelated amino acid scan (e.g. alanine scan) is carried out using HTRF. In one embodiment, the unrelated amino acid scan (e.g. alanine scan) is carried out using ELISA. When the alanine scan is carried out with either ELISA or HTRF, an amino acid residue is identified as contributing to the epitope if the reduction in signal is at least 25%. In one embodiment, the reduction in signal is at least 30%. In one embodiment, the reduction in signal is at least 35%. In one embodiment, the reduction in signal is at least 40%. In one embodiment, the reduction in signal is at least 45%. In one embodiment, the reduction in signal is at least 50%. In one embodiment, the reduction in signal is at least 55%. In one embodiment, the reduction in signal is at least 60%. In one embodiment, the reduction in signal is at least 70%. In one embodiment, the reduction in signal is at least 75%. In one embodiment, the reduction in signal is at least 80%. In one embodiment, the reduction in signal is at least 85%. In one embodiment, the reduction in signal is at least 90%.

When the alanine scan is carried out with SPR, an amino acid residue is identified as contributing to the epitope if there is at least a 10-fold reduction in affinity. In one embodiment, the reduction in affinity is at least 15 fold. In one embodiment, the reduction in affinity is at least 20 fold. In one embodiment, the reduction in affinity is at least 30 fold. In one embodiment, the reduction in affinity is at least 40 fold. In one embodiment, the reduction in affinity is at least 50 fold. In one embodiment, the reduction in affinity is at least 100 fold. In one embodiment, the contact residues of the epitope are identified by X-ray crystallography. In one embodiment, the contact residues of the epitope are identified by cryo-electro microscopy. In one embodiment, the contact residues of the epitope are identified by a combination of limited proteolytic digestion and mass spectrometry.

Concept 14. The antibody or fragment according to concept 13, wherein the epitope is identified by unrelated amino acid scanning, or by X-ray crystallography.

Concept 15. The antibody or fragment according to concept 14, wherein the contact residues of the epitope are defined by a reduction in affinity of at least 10-fold in an unrelated amino acid scan, e.g. an alanine scan as determined by SPR. In one embodiment, the reduction in affinity is at least 15 fold. In one embodiment, the reduction in affinity is at least 20 fold. In one embodiment, the reduction in affinity is at least 30 fold. In one embodiment, the reduction in affinity is at least 40 fold. In one embodiment, the reduction in affinity is at least 50 fold. In one embodiment, the reduction in affinity is at least 100 fold. SPR may be carried out as described hereinabove.

Concept 16. An antibody or fragment thereof which competes for binding to hPD-L1 with the antibody 1D05.

Competition may be determined by surface plasmon resonance (SPR), such techniques being readily apparent to the skilled person. SPR may be carried out using Biacore™, Proteon™ or another standard SPR technique. Such competition may be due, for example, to the antibodies or fragments binding to identical or overlapping epitopes of hPD-L1. In one embodiment, competition is determined by ELISA, such techniques being readily apparent to the skilled person. In one embodiment, competition is determined by homogenous time resolved fluorescence (HTRF), such techniques being readily apparent to the skilled person. In one embodiment, competition is determined by fluorescence activated cell sorting (FACS), such techniques being readily apparent to the skilled person. In one embodiment, competition is determined by ForteBio Octet® Bio-Layer Interferometry (BLI) such techniques being readily apparent to the skilled person.

In one embodiment, the antibody or fragment competes (e.g., in a dose-dependent manner) with hPD-1 (or a fusion protein thereof) for binding to cell surface-expressed hPD-L1. In one embodiment, the antibody or fragment competes (e.g., in a dose-dependent manner) with hPD-1 (or a fusion protein thereof) for binding to soluble hPDL-1. In one embodiment, the antibody or fragment partially or completely inhibits binding of PD-1 and/or CD80 to cell surface-expressed PD-L1, such as hPD-L1. In another embodiment, the antibody or fragment partially or completely inhibits binding of hPD-1 and/or CD80 to soluble hPD-L1. In some embodiments, the antibody or fragment partially or completely increases the secretion of IFNγ, CD25 and IL-2 from a cell having cell surface-expressed PD-1. In one embodiment, the antibody or fragment partially or completely inhibits binding of CD80 to soluble hPD-L1, but does not show any detectable inhibition of the binding of PD-1 to cell surface-expressed PD-L1. In one embodiment, the antibody or fragment partially or completely inhibits binding of CD80 to soluble hPD-L1, but does not show any detectable inhibition of the binding of PD-1 to soluble PD-L1.

As used herein, “inhibits”, “inhibition”, “inhibiting” and the like, as used herein refers to the ability of an antagonist (e.g. an antibody or fragment thereof) to bind to an epitope which either partially or completely prevents the binding of the receptor (e.g. CD80 or PD-1) to the ligand (e.g. PD-L1). If the epitope to which the antagonist binds completely blocks the binding site of the ligand, then ligand binding is completely prevented (which may be a physical blocking—in the case of overlapping epitopes—or steric blocking—where the antagonist is large such that it prevents the ligand binding to its distinct epitope), and the ligand is not removed from circulation. The concentration of circulating ligand may therefore appear to be increased. If the epitope to which the antagonist binds partially blocks the binding site of the ligand, the ligand may be able to bind, but only weakly (in the case of partial inhibition), or in a different orientation to the natural binding interaction. In this case, some of the ligand may be removed from circulation, but not as much as when the ligand binding site is completely free and available for binding. Inhibition thus refers to the physical interaction of ligand and receptor. Inhibition can be measured by HTRF, which is described in more detail elsewhere herein and in Mathis (1995) Clinical Chemistry 41(9), 1391-1397. Inhibition can also be measured by flow cytometry, where receptor is expressed on cells, or by ELISA, where receptor is adsorbed onto plates.

Concept 16a. An antibody or fragment thereof which competes for binding to hPD-L1 with the antibody 84G09.

Concept 16b. An antibody or fragment thereof which competes for binding to hPD-L1 with the antibody 411B08.

Concept 16c. An antibody or fragment thereof which competes for binding to hPD-L1 with the antibody 411C04.

Concept 16d. An antibody or fragment thereof which competes for binding to hPD-L1 with the antibody 411D07.

Concept 16e. An antibody or fragment thereof which competes for binding to hPD-L1 with the antibody 385F01.

Concept 16f. An antibody or fragment thereof which competes for binding to hPD-L1 with the antibody 386H03.

Concept 16g. An antibody or fragment thereof which competes for binding to hPD-L1 with the antibody 389A03.

Concept 16h. An antibody or fragment thereof which competes for binding to hPD-L1 with the antibody 413D08.

Concept 16i. An antibody or fragment thereof which competes for binding to hPD-L1 with the antibody 413G05.

Concept 16j. An antibody or fragment thereof which competes for binding to hPD-L1 with the antibody 413F09.

Concept 16k. An antibody or fragment thereof which competes for binding to hPD-L1 with the antibody 414B06.

Concept 16l. An antibody or fragment thereof which competes for binding to hPD-L1 with the antibody 416E01.

The antibodies have the sequences as described hereinabove.

Concept 17. The antibody or fragment according to any one of concepts 10 to 16, wherein the V_{H} domain comprises the CDRH3 sequence of SEQ ID NO:29 or 32, or the CDRH3 sequence of SEQ ID NO:29 or 32 comprising 6 or fewer amino acid substitutions. Concept 17a: An antibody or a fragment thereof according to any one of concepts 10 to 16 (but when dependent on concept 13, it is dependent on concept 13a, and when dependent on concept 16, it is dependent on concept 16a), wherein the V_{H} domain comprises the CDRH3 sequence of SEQ ID NO:9 or 12, or the CDRH3 sequence of SEQ ID NO:9 or 12 comprising 6 or fewer amino acid substitutions.

Concept 17b: An antibody or a fragment thereof according to any one of concepts 10 to 16 (but when dependent on concept 13, it is dependent on concept 13b, and when dependent on concept 16, it is dependent on concept 16b), wherein the V_{H} domain comprises the CDRH3 sequence of SEQ ID NO:54 or 57, or the CDRH3 sequence of SEQ ID NO: 54 or 57 comprising 6 or fewer amino acid substitutions.

Concept 17c: An antibody or a fragment thereof according to any one of concepts 10 to 16 (but when dependent on concept 13, it is dependent on concept 13c, and when dependent on concept 16, it is dependent on concept 16c), wherein the a V_{H} domain comprises the CDRH3 sequence of SEQ ID NO:74 or 77, or the CDRH3 sequence of SEQ ID NO: 74 or 77 comprising 6 or fewer amino acid substitutions.

Concept 17d: An antibody or a fragment thereof according to any one of concepts 10 to 16 (but when dependent on concept 13, it is dependent on concept 13d, and when dependent on concept 16, it is dependent on concept 16d), wherein the V_{H} domain comprises the CDRH3 sequence of SEQ ID NO:94 or 97, or the CDRH3 sequence of SEQ ID NO: 94 or 97 comprising 3 or fewer amino acid substitutions.

Concept 17e: An antibody or a fragment thereof according to any one of concepts 10 to 16 (but when dependent on concept 13, it is dependent on concept 13e, and when dependent on concept 16, it is dependent on concept 16e), wherein the V_{H} domain comprises the CDRH3 sequence of SEQ ID NO:114 or 117, or the CDRH3 sequence of SEQ ID NO: 114 or 117 comprising 6 or fewer amino acid substitutions.

Concept 17f: An antibody or a fragment thereof according to any one of concepts 10 to 16 (but when dependent on concept 13, it is dependent on concept 13f, and when dependent on concept 16, it is dependent on concept 16f), wherein the V_{H} domain comprises the CDRH3 sequence of SEQ ID NO:144 or 147, or the CDRH3 sequence of SEQ ID NO: 144 or 147 comprising 3 or fewer amino acid substitutions.

Concept 17g: An antibody or a fragment thereof according to any one of concepts 10 to 16 (but when dependent on concept 13, it is dependent on concept 13g, and when dependent on concept 16, it is dependent on concept 16g), wherein the V_{H} domain comprises the CDRH3 sequence of SEQ ID NO:174 or 177, or the CDRH3 sequence of SEQ ID NO: 174 or 177 comprising 6 or fewer amino acid substitutions.

Concept 17h: An antibody or a fragment thereof according to any one of concepts 10 to 16 (but when dependent on concept 13, it is dependent on concept 13h, and when dependent on concept 16, it is dependent on concept 16h), wherein the V_{H} domain comprises the CDRH3 sequence of SEQ ID NO134 or 137, or the CDRH3 sequence of SEQ ID NO: 134 or 137 comprising 5 or fewer amino acid substitutions.

Concept 17i: An antibody or a fragment thereof according to any one of concepts 10 to 16 (but when dependent on concept 13, it is dependent on concept 13i, and when dependent on concept 16, it is dependent on concept 16i), wherein the V_{H} domain comprises the CDRH3 sequence of SEQ ID NO:240 or 243, or the CDRH3 sequence of SEQ ID NO: 240 or 243 comprising 6 or fewer amino acid substitutions.

Concept 17j: An antibody or a fragment thereof according to any one of concepts 10 to 16 (but when dependent on concept 13, it is dependent on concept 13j, and when dependent on concept 16, it is dependent on concept 16j), wherein the a V_{H} domain comprises the CDRH3 sequence of SEQ ID NO:260 or 263, or the CDRH3 sequence of SEQ ID NO: 260 or 263 comprising 6 or fewer amino acid substitutions.

Concept 17k: An antibody or a fragment thereof according to any one of concepts 10 to 16 (but when dependent on concept 13, it is dependent on concept 13k, and when dependent on concept 16, it is dependent on concept 16k), wherein the V_{H} domain comprises the CDRH3 sequence of SEQ ID NO:280 or 283, or the CDRH3 sequence of SEQ ID NO: 280 or 283 comprising 6 or fewer amino acid substitutions.

Concept 17l: An antibody or a fragment thereof according to any one of concepts 10 to 16 (but when dependent on concept 13, it is dependent on concept 13l, and when dependent on concept 16, it is dependent on concept 16l), wherein the V_{H} domain comprises the CDRH3 sequence of SEQ ID NO:345 or 348, or the CDRH3 sequence of SEQ ID NO: 345 or 348 comprising 6 or fewer amino acid substitutions.

Concept 18. The antibody or fragment according to any preceding concept, wherein the V_{H} domain comprises the CDRH1 sequence of SEQ ID NO:27 or 30 or the CDRH1 sequence of SEQ ID NO:27 or 30 comprising 3, 2 or 1 amino acid substitution(s).

Concept 18a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, and when dependent on concept 17, it is dependent on concept 17a), wherein the V_{H} domain comprises the CDRH1 sequence of SEQ ID NO: 7 or 10, or the CDRH1 sequence of SEQ ID NO: 7 or 10 comprising 3, 2 or 1 amino acid substitution(s).

Concept 18b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, and when dependent on concept 17, it is dependent on concept 17b), wherein the V_{H} domain comprises the CDRH1 sequence of SEQ ID NO: 52 or 55, or the CDRH1 sequence of SEQ ID NO: 52 or 55 comprising 3, 2 or 1 amino acid substitution(s).

Concept 18c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, and when dependent on concept 17, it is dependent on concept 17c), wherein the V_{H} domain comprises the CDRH1 sequence of SEQ ID NO: 72 or 75, or the CDRH1 sequence of SEQ ID NO: 72 or 75 comprising 3, 2 or 1 amino acid substitution(s).

Concept 18d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, and when dependent on concept 17, it is dependent on concept 17d), wherein the V_{H} domain comprises the CDRH1 sequence of SEQ ID NO: 92 or 95, or the CDRH1 sequence of SEQ ID NO: 92 or 95 comprising 3, 2 or 1 amino acid substitution(s).

Concept 18e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, and when dependent on concept 17, it is dependent on concept 17e), wherein the V_{H} domain comprises the CDRH1 sequence of SEQ ID NO: 112 or 115, or the CDRH1 sequence of SEQ ID NO:112 or 115 comprising 3, 2 or 1 amino acid substitution(s).

Concept 18f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, and when dependent on concept 17, it is dependent on concept 17f), wherein the V_{H} domain comprises the CDRH1 sequence of SEQ ID NO: 142 or 145, or the CDRH1 sequence of SEQ ID NO: 142 or 145 comprising 3, 2 or 1 amino acid substitution(s).

Concept 18g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, and when dependent on concept 17, it is dependent on concept 17g), wherein the V_{H} domain comprises the CDRH1 sequence of SEQ ID NO: 172 or 175, or the CDRH1 sequence of SEQ ID NO: 172 or 175 comprising 3, 2 or 1 amino acid substitution(s).

Concept 18h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, and when dependent on concept 17, it is dependent on concept 17h), wherein the V_{H} domain comprises the CDRH1 sequence of SEQ ID NO:132 or 135, or the CDRH1 sequence of SEQ ID NO:132 or 135 comprising 3, 2 or 1 amino acid substitution(s).

Concept 18i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, and when dependent on concept 17, it is dependent on concept 17i), wherein the V_{H} domain comprises the CDRH1 sequence of SEQ ID NO: 238 or 241, or the CDRH1 sequence of SEQ ID NO: 238 or 241 comprising 3, 2 or 1 amino acid substitution(s).

Concept 18j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, and when dependent on concept 17, it is dependent on concept 17j), wherein the V_{H} domain comprises the CDRH1 sequence of SEQ ID NO: 258 or 261, or the CDRH1 sequence of SEQ ID NO: 258 or 261 comprising 3, 2 or 1 amino acid substitution(s).

Concept 18k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, and when dependent on concept 17, it is dependent on concept 17k), wherein the V_{H} domain comprises the CDRH1 sequence of SEQ ID NO: 278 or 281, or the CDRH1 sequence of SEQ ID NO: 278 or 281 comprising 3, 2 or 1 amino acid substitution(s).

Concept 18l: An antibody or a fragment thereof according to any preceding concept (but when dependent on concept 9, it is dependent on concept 91, when dependent on concept 13, it is dependent on concept 13l, when dependent on concept 16, it is dependent on concept 16l, and when dependent on concept 17, it is dependent on concept 17l), wherein the V_{H} domain comprises the CDRH1 sequence of SEQ ID NO: 343 or 346, or the CDRH1 sequence of SEQ ID NO: 343 or 346 comprising 3, 2 or 1 amino acid substitution(s).

Concept 19. The antibody or fragment according to any preceding concept, wherein the V_{H} domain comprises the CDRH2 sequence of SEQ ID NO:28 or 31, or the CDRH2 sequence of SEQ ID NO:28 or 31 comprising 4 or fewer amino acid substitutions.

Concept 19a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, and when dependent on concept 18, it is dependent on concept 18a), wherein the V_{H} domain comprises the CDRH2 sequence of SEQ ID NO: 8 or 11, or the CDRH2 sequence of SEQ ID NO: 8 or 11 comprising 4 or fewer amino acid substitutions.

Concept 19b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, and when dependent on concept 18, it is dependent on concept 18b), wherein the V_{H} domain comprises the CDRH2 sequence of SEQ ID NO:53 or 56, or the CDRH2 sequence of SEQ ID NO: 53 or 56 comprising 3, 2 or 1 amino acid substitution(s).

Concept 19c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, and when dependent on concept 18, it is dependent on concept 18c), wherein the V_{H} domain comprises the CDRH2 sequence of SEQ ID NO:73 or 76, or the CDRH2 sequence of SEQ ID NO:73 or 76 comprising 3, 2 or 1 amino acid substitution(s).

Concept 19d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, and when dependent on concept 18, it is dependent on concept 18d), wherein the V_{H} domain comprises the CDRH2 sequence of SEQ ID NO:93 or 96, or the CDRH2 sequence of SEQ ID NO: 93 or 96 comprising 3, 2 or 1 amino acid substitution(s).

Concept 19e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, and when dependent on concept 18, it is dependent on concept 18e), wherein the V_{H} domain comprises the CDRH2 sequence of SEQ ID NO:113 or 116, or the CDRH2 sequence of SEQ ID NO: 113 or 116 comprising 3, 2 or 1 amino acid substitution(s).

Concept 19f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, and when dependent on concept 18, it is dependent on concept 18f), wherein the V_{H} domain comprises the CDRH2 sequence of SEQ ID NO:143 or 146, or the CDRH2 sequence of SEQ ID NO: 143 or 146 comprising 3, 2 or 1 amino acid substitution(s).

Concept 19g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, and when dependent on concept 18, it is dependent on concept 18g), wherein the V_{H} domain comprises the CDRH2 sequence of SEQ ID NO:173 or 176, or the CDRH2 sequence of SEQ ID NO: 173 or 176 comprising 3, 2 or 1 amino acid substitution(s).

Concept 19h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, and when dependent on concept 18, it is dependent on concept 18h), wherein the V_{H} domain comprises the CDRH2 sequence of SEQ ID NO:133 or 136, or the CDRH2 sequence of SEQ ID NO: 133 or 136 comprising 3, 2 or 1 amino acid substitution(s).

Concept 19i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, and when dependent on concept 18, it is dependent on concept 18i), wherein the V_{H} domain comprises the CDRH2 sequence of SEQ ID NO:239 or 242, or the CDRH2 sequence of SEQ ID NO: 239 or 242 comprising 3, 2 or 1 amino acid substitution(s).

Concept 19j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, and when dependent on concept 18, it is dependent on concept 18j), wherein the V_{H} domain comprises the CDRH2 sequence of SEQ ID NO:259 or 262, or the CDRH2 sequence of SEQ ID NO: 259 or 262 comprising 3, 2 or 1 amino acid substitution(s).

Concept 19k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, and when dependent on concept 18, it is dependent on concept 18k), wherein the V_{H} domain comprises the CDRH2 sequence of SEQ ID NO:279 or 282, or the CDRH2 sequence of SEQ ID NO: 279 or 282 comprising 3, 2 or 1 amino acid substitution(s).

Concept 19l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, and when dependent on concept 18, it is dependent on concept 18l), wherein the V_{H} domain comprises the CDRH2 sequence of SEQ ID NO:344 or 347, or the CDRH2 sequence of SEQ ID NO: 344 or 347 comprising 3, 2 or 1 amino acid substitution(s).

Concept 20. The antibody or fragment according to any preceding concept, wherein the V_{H} domain comprises an amino acid sequence of SEQ ID NO:33, or a heavy chain variable domain amino acid sequence that is at least 80% (e.g. at least 85%, or at least 90%) identical to SEQ ID NO:33.

Concept 20a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, when dependent on concept 18, it is dependent on concept 18a, and when dependent on concept 19, it is dependent on concept 19a), wherein the V_{H} domain comprises an amino acid sequence of SEQ ID NO:13, or a heavy chain variable domain amino acid sequence that is at least 80% (e.g. at least 85%, or at least 90%) identical to SEQ ID NO:13.

Concept 20b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, when dependent on concept 18, it is dependent on concept 18b, and when dependent on concept 19, it is dependent on concept 19b), wherein the V_{H} domain comprises an amino acid sequence of SEQ ID NO:58, or a heavy chain variable domain amino acid sequence that is at least 80% (e.g. at least 85%, or at least 90%) identical to SEQ ID NO:58.

Concept 20c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, when dependent on concept 18, it is dependent on concept 18c, and when dependent on concept 19, it is dependent on concept 19c), wherein the V_{H} domain comprises an amino acid sequence of SEQ ID NO:78, or a heavy chain variable domain amino acid sequence that is at least 80% (e.g. at least 85%, or at least 90%) identical to SEQ ID NO:78.

Concept 20d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, when dependent on concept 18, it is dependent on concept 18d, and when dependent on concept 19, it is dependent on concept 19d), wherein the V_{H} domain comprises an amino acid sequence of SEQ ID NO:98, or a heavy chain variable domain amino acid sequence that is at least 80% (e.g. at least 85%, or at least 90%) identical to SEQ ID NO:98.

Concept 20e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, when dependent on concept 18, it is dependent on concept 18e, and when dependent on concept 19, it is dependent on concept 19e), wherein the V_{H} domain comprises an amino acid sequence of SEQ ID NO:118, or a heavy chain variable domain amino acid sequence that is at least 80% (e.g. at least 85%, or at least 90%) identical to SEQ ID NO:118.

Concept 20f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, when dependent on concept 18, it is dependent on concept 18f, and when dependent on concept 19, it is dependent on concept 19f), wherein the V_{H} domain comprises an amino acid sequence of SEQ ID NO:158, or a heavy chain variable domain amino acid sequence that is at least 80% (e.g. at least 85%, or at least 90%) identical to SEQ ID NO:158.

Concept 20g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, when dependent on concept 18, it is dependent on concept 18g, and when dependent on concept 19, it is dependent on concept 19g), wherein the V_{H} domain comprises an amino acid sequence of SEQ ID NO:178, or a heavy chain variable domain amino acid sequence that is at least 80% (e.g. at least 85%, or at least 90%) identical to SEQ ID NO:178.

Concept 20h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, when dependent on concept 18, it is dependent on concept 18h, and when dependent on concept 19, it is dependent on concept 19h), wherein the V_{H} domain comprises an amino acid sequence of SEQ ID NO:138, or a heavy chain variable domain amino acid sequence that is at least 80% (e.g. at least 85%, or at least 90%) identical to SEQ ID NO:138.

Concept 20i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, when dependent on concept 18, it is dependent on concept 18i, and when dependent on concept 19, it is dependent on concept 19i), wherein the V_{H} domain comprises an amino acid sequence of SEQ ID NO:244, or a heavy chain variable domain amino acid sequence that is at least 80% (e.g. at least 85%, or at least 90%) identical to SEQ ID NO:244.

Concept 20j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, when dependent on concept 18, it is dependent on concept 18j, and when dependent on concept 19, it is dependent on concept 19j), wherein the V_{H} domain comprises an amino acid sequence of SEQ ID NO:264, or a heavy chain variable domain amino acid sequence that is at least 80% (e.g. at least 85%, or at least 90%) identical to SEQ ID NO:264.

Concept 20k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, when dependent on concept 18, it is dependent on concept 18k, and when dependent on concept 19, it is dependent on concept 19k), wherein the V_{H} domain comprises an amino acid sequence of SEQ ID NO:284, or a heavy chain variable domain amino acid sequence that is at least 80% (e.g. at least 85%, or at least 90%) identical to SEQ ID NO:284.

Concept 20l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, when dependent on concept 18, it is dependent on concept 18l, and when dependent on concept 19, it is dependent on concept 19l), wherein the V_{H} domain comprises an amino acid sequence of SEQ ID NO:349, or a heavy chain variable domain amino acid sequence that is at least 80% (e.g. at least 85%, or at least 90%) identical to SEQ ID NO:349.

In one embodiment, the amino acid sequence is at least 70% identical to the specified Seq ID No. In one embodiment, the amino acid sequence is at least 75% identical to the specified Seq ID No. In one embodiment, the amino acid sequence is at least 95% identical to the specified Seq ID No. In one embodiment, the amino acid sequence is at least 96% identical to the specified Seq ID No. In one embodiment, the amino acid sequence is at least 97% identical to the specified Seq ID No. In one embodiment, the amino acid sequence is at least 98% identical to the specified Seq ID No. In one embodiment, the amino acid sequence is at least 99% identical to the specified Seq ID No. In one embodiment, the amino acid sequence is at least 99.5% identical to the specified Seq ID No.

Concept 21. The antibody or fragment according to any preceding concept comprising first and second copies of said V_{H} domain.

Concept 22. The antibody or fragment according to any preceding concept, comprising a V_{L} domain which comprises the CDRL1 sequence of SEQ ID NO:37 or 40, or the CRDL1 sequence of SEQ ID NO:37 or 40 comprising 3 or fewer amino acid substitutions.

Concept 22a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, when dependent on concept 18, it is dependent on concept 18a, when dependent on concept 19, it is dependent on concept 19a, and when dependent on concept 20, it is dependent on concept 20a), comprising a V_{L} domain, which comprises the CDRL1 sequence of SEQ ID NO: 17 or 20, or the CDRL1 sequence of SEQ ID NO:17 or 20 comprising 3 or fewer amino acid substitutions.

Concept 22b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, when dependent on concept 18, it is dependent on concept 18b, when dependent on concept 19, it is dependent on concept 19b, and when dependent on concept 20, it is dependent on concept 20b), comprising a V_{L} domain which comprises the CDRL1 sequence of SEQ ID NO: 62 or 65, or the CDRL1 sequence of SEQ ID NO:62 or 65 comprising 3 or fewer amino acid substitutions.

Concept 22c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, when dependent on concept 18, it is dependent on concept 18c, when dependent on concept 19, it is dependent on concept 19c, and when dependent on concept 20, it is dependent on concept 20c), comprising a V_{L} domain which comprises the CDRL1 sequence of SEQ ID NO: 82 or 85, or the CDRL1 sequence of SEQ ID NO:82 or 85 comprising 2 or 1 amino acid substitution(s).

Concept 22d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, when dependent on concept 18, it is dependent on concept 18d, when dependent on concept 19, it is dependent on concept 19d, and when dependent on concept 20, it is dependent on concept 20d), comprising a V_{L} domain which comprises the CDRL1 sequence of SEQ ID NO: 102 or 10⁵, or the CDRL1 sequence of SEQ ID NO:102 or 10⁵ comprising 5 or fewer amino acid substitutions.

Concept 22e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, when dependent on concept 18, it is dependent on concept 18e, when dependent on concept 19, it is dependent on concept 19e, and when dependent on concept 20, it is dependent on concept 20e), comprising a V_{L} domain which comprises the CDRL1 sequence of SEQ ID NO: 122 or 125, or the CDRL1 sequence of SEQ ID NO:122 or 125 comprising 2 or 1 amino acid substitution(s).

Concept 22f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, when dependent on concept 18, it is dependent on concept 18f, when dependent on concept 19, it is dependent on concept 19f, and when dependent on concept 20, it is dependent on concept 20f), comprising a V_{L} domain which comprises the CDRL1 sequence of SEQ ID NO:162 or 165, or the CDRL1 sequence of SEQ ID NO:162 or 165 comprising 5 or fewer amino acid substitutions.

Concept 22g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, when dependent on concept 18, it is dependent on concept 18g, when dependent on concept 19, it is dependent on concept 19g, and when dependent on concept 20, it is dependent on concept 20g), comprising a V_{L} domain which comprises the CDRL1 sequence of SEQ ID NO: 182 or 185, or the CDRL1 sequence of SEQ ID NO:182 or 185 comprising 5 or fewer amino acid substitutions.

Concept 22h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, when dependent on concept 18, it is dependent on concept 18h, when dependent on concept 19, it is dependent on concept 19h, and when dependent on concept 20, it is dependent on concept 20h), comprising a V_{L} domain which comprises the CDRL1 sequence of SEQ ID NO: 142 or 145, or the CDRL1 sequence of SEQ ID NO:142 or 145 comprising 2 or 1 amino acid substitution(s).

Concept 22i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, when dependent on concept 18, it is dependent on concept 18i, when dependent on concept 19, it is dependent on concept 19i, and when dependent on concept 20, it is dependent on concept 20i), comprising a V_{L} domain which comprises the CDRL1 sequence of SEQ ID NO:248 or 251, or the CDRL1 sequence of SEQ ID NO:248 or 251 comprising 2 or 1 amino acid substitution(s).

Concept 22j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, when dependent on concept 18, it is dependent on concept 18j, when dependent on concept 19, it is dependent on concept 19j, and when dependent on concept 20, it is dependent on concept 20j), comprising a V_{L} domain which comprises the CDRL1 sequence of SEQ ID NO:268 or 271, or the CDRL1 sequence of SEQ ID NO:268 or 271 comprising 2 or 1 amino acid substitution(s).

Concept 22k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, when dependent on concept 18, it is dependent on concept 18k, when dependent on concept 19, it is dependent on concept 19k, and when dependent on concept 20, it is dependent on concept 20k), comprising a V_{L} domain which comprises the CDRL1 sequence of SEQ ID NO: 288 or 291, or the CDRL1 sequence of SEQ ID NO:288 or 291 comprising 2 or 1 amino acid substitution(s).

Concept 22l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, when dependent on concept 18, it is dependent on concept 18l, when dependent on concept 19, it is dependent on concept 19l, and when dependent on concept 20, it is dependent on concept 201), comprising a V_{L} domain which comprises the CDRL1 sequence of SEQ ID NO:353 or 356, or the CDRL1 sequence of SEQ ID NO:353 or 356 comprising 2 or 1 amino acid substitution(s).

Concept 23. The antibody or fragment according to any preceding concept, comprising a or said V_{L} domain, which V_{L} domain comprises the CDRL2 sequence of SEQ ID NO: 38 or 41, or the CRDL2 sequence of SEQ ID NO:38 or 41 comprising 2 or 1 amino acid substitution(s), for example a CDRL2 sequence of Seq ID No:50.

Concept 23a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, when dependent on concept 18, it is dependent on concept 18a, when dependent on concept 19, it is dependent on concept 19a, when dependent on concept 20, it is dependent on concept 20a, and when dependent on concept 22, it is dependent on concept 22a), comprising a or said V_{L} domain, which V_{L} domain comprises the CDRL2 sequence of SEQ ID NO:18 or 21, or the CDRL2 sequence of SEQ ID NO:18 or 21 comprising 2 or 1 amino acid substitution(s).

Concept 23b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, when dependent on concept 18, it is dependent on concept 18b, when dependent on concept 19, it is dependent on concept 19b, when dependent on concept 20, it is dependent on concept 20b, and when dependent on concept 22, it is dependent on concept 22b), comprising a or said V_{L} domain, which V_{L} domain comprises the CDRL2 sequence of SEQ ID NO:63 or 66, or the CDRL2 sequence of SEQ ID NO:63 or 66 comprising one amino acid substitution.

Concept 23c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, when dependent on concept 18, it is dependent on concept 18c, when dependent on concept 19, it is dependent on concept 19c, when dependent on concept 20, it is dependent on concept 20c, and when dependent on concept 22, it is dependent on concept 22c), comprising a or said V_{L} domain, which V_{L} domain comprises the CDRL2 sequence of SEQ ID NO:83 or 86, or the CDRL2 sequence of SEQ ID NO:83 or 86 comprising one amino acid substitution.

Concept 23d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, when dependent on concept 18, it is dependent on concept 18d, when dependent on concept 19, it is dependent on concept 19d, when dependent on concept 20, it is dependent on concept 20d, and when dependent on concept 22, it is dependent on concept 22d), comprising a or said V_{L} domain, which V_{L} domain comprises the CDRL2 sequence of SEQ ID NO:103 or 106, or the CDRL2 sequence of SEQ ID NO:103 or 106 comprising one amino acid substitution.

Concept 23e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, when dependent on concept 18, it is dependent on concept 18e, when dependent on concept 19, it is dependent on concept 19e, when dependent on concept 20, it is dependent on concept 20e, and when dependent on concept 22, it is dependent on concept 22e), comprising a or said V_{L} domain, which V_{L} domain comprises the CDRL2 sequence of SEQ ID NO:123 or 126, or the CDRL2 sequence of SEQ ID NO:123 or 126 comprising one amino acid substitution.

Concept 23f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, when dependent on concept 18, it is dependent on concept 18f, when dependent on concept 19, it is dependent on concept 19f, when dependent on concept 20, it is dependent on concept 20f, and when dependent on concept 22, it is dependent on concept 22f), comprising a or said V_{L} domain, which V_{L} domain comprises the CDRL2 sequence of SEQ ID NO:153 or 156, or the CDRL2 sequence of SEQ ID NO:153 or 156 comprising one amino acid substitution.

Concept 23g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, when dependent on concept 18, it is dependent on concept 18g, when dependent on concept 19, it is dependent on concept 19g, when dependent on concept 20, it is dependent on concept 20g, and when dependent on concept 22, it is dependent on concept 22g), comprising a or said V_{L} domain, which V_{L} domain comprises the CDRL2 sequence of SEQ ID NO:183 or 186, or the CDRL2 sequence of SEQ ID NO:183 or 186 comprising one amino acid substitution.

Concept 23h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, when dependent on concept 18, it is dependent on concept 18h, when dependent on concept 19, it is dependent on concept 19h, when dependent on concept 20, it is dependent on concept 20h, and when dependent on concept 22, it is dependent on concept 22h), comprising a or said V_{L} domain, which V_{L} domain comprises the CDRL2 sequence of SEQ ID NO:143 or 146, or the CDRL2 sequence of SEQ ID NO:143 or 146 comprising one amino acid substitution.

Concept 23i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, when dependent on concept 18, it is dependent on concept 18i, when dependent on concept 19, it is dependent on concept 19i, when dependent on concept 20, it is dependent on concept 20i, and when dependent on concept 22, it is dependent on concept 22i), comprising a or said V_{L} domain, which V_{L} domain comprises the CDRL2 sequence of SEQ ID NO:249 or 252, or the CDRL2 sequence of SEQ ID NO:249 or 252 comprising one amino acid substitution.

Concept 23j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, when dependent on concept 18, it is dependent on concept 18j, when dependent on concept 19, it is dependent on concept 19j, when dependent on concept 20, it is dependent on concept 20j, and when dependent on concept 22, it is dependent on concept 22j), comprising a or said V_{L} domain, which V_{L} domain comprises the CDRL2 sequence of SEQ ID NO:269 or 272, or the CDRL2 sequence of SEQ ID NO:269 or 272 comprising one amino acid substitution.

Concept 23k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, when dependent on concept 18, it is dependent on concept 18k, when dependent on concept 19, it is dependent on concept 19k, when dependent on concept 20, it is dependent on concept 20k, and when dependent on concept 22, it is dependent on concept 22k), comprising a or said V_{L} domain, which V_{L} domain comprises the CDRL2 sequence of SEQ ID NO:289 or 292, or the CDRL2 sequence of SEQ ID NO:289 or 292 comprising one amino acid substitution.

Concept 23l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, when dependent on concept 18, it is dependent on concept 18l, when dependent on concept 19, it is dependent on concept 19l, when dependent on concept 20, it is dependent on concept 20l, and when dependent on concept 22, it is dependent on concept 22l), comprising a or said V_{L} domain, which V_{L} domain comprises the CDRL2 sequence of SEQ ID NO:354 or 357, or the CDRL2 sequence of SEQ ID NO:354 or 357 comprising one amino acid substitution.

Concept 24. The antibody or fragment according to any preceding concept, comprising a or said V_{L} domain, which V_{L} domain comprises the CDRL3 sequence of SEQ ID NO: 39 or 42, or the CRDL3 sequence of SEQ ID NO:39 or 42 comprising 4 or fewer amino acid substitutions.

Concept 24a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, when dependent on concept 18, it is dependent on concept 18a, when dependent on concept 19, it is dependent on concept 19a, when dependent on concept 20, it is dependent on concept 20a, when dependent on concept 22, it is dependent on concept 22a, and when dependent on concept 23, it is dependent on concept 23a), comprising a or said V_{L} domain, which V_{L} domain comprises the CDRL3 sequence of SEQ ID NO:19 or 22, or the CDRL3 sequence of SEQ ID NO: 19 or 22 comprising 4 or fewer amino acid substitutions.

Concept 24b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, when dependent on concept 18, it is dependent on concept 18b, when dependent on concept 19, it is dependent on concept 19b, when dependent on concept 20, it is dependent on concept 20b, when dependent on concept 22, it is dependent on concept 22b, and when dependent on concept 23, it is dependent on concept 23b), comprising a or said V_{L} domain, which V_{L} domain comprises the CDRL3 sequence of SEQ ID NO:64 or 67, or the CDRL3 sequence of SEQ ID NO:64 or 67 comprising 4 or fewer amino acid substitutions.

Concept 24c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, when dependent on concept 18, it is dependent on concept 18c, when dependent on concept 19, it is dependent on concept 19c, when dependent on concept 20, it is dependent on concept 20c, when dependent on concept 22, it is dependent on concept 22c, and when dependent on concept 23, it is dependent on concept 23c), comprising a or said V_{L} domain, which V_{L} domain comprises the CDRL3 sequence of SEQ ID NO:84 or 87, or the CDRL3 sequence of SEQ ID NO:84 or 87 comprising 4 or fewer amino acid substitutions.

Concept 24d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, when dependent on concept 18, it is dependent on concept 18d, when dependent on concept 19, it is dependent on concept 19d, when dependent on concept 20, it is dependent on concept 20d, when dependent on concept 22, it is dependent on concept 22d, and when dependent on concept 23, it is dependent on concept 23d), comprising a or said V_{L} domain, which V_{L} domain comprises the CDRL3 sequence of SEQ ID NO:104 or 107, or the CDRL3 sequence of SEQ ID NO:104 or 107 comprising 4 or fewer amino acid substitutions.

Concept 24e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, when dependent on concept 18, it is dependent on concept 18e, when dependent on concept 19, it is dependent on concept 19e, when dependent on concept 20, it is dependent on concept 20e, when dependent on concept 22, it is dependent on concept 22e, and when dependent on concept 23, it is dependent on concept 23e), comprising a or said V_{L} domain, which V_{L} domain comprises the CDRL3 sequence of SEQ ID NO:124 or 127, or the CDRL3 sequence of SEQ ID NO:124 or 127 comprising 4 or fewer amino acid substitutions.

Concept 24f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, when dependent on concept 18, it is dependent on concept 18f, when dependent on concept 19, it is dependent on concept 19f, when dependent on concept 20, it is dependent on concept 20f, when dependent on concept 22, it is dependent on concept 22f, and when dependent on concept 23, it is dependent on concept 23f), comprising a or said V_{L} domain, which V_{L} domain comprises the CDRL3 sequence of SEQ ID NO:164 or 167, or the CDRL3 sequence of SEQ ID NO:164 or 167 comprising 4 or fewer amino acid substitutions.

Concept 24g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, when dependent on concept 18, it is dependent on concept 18g, when dependent on concept 19, it is dependent on concept 19g, when dependent on concept 20, it is dependent on concept 20g, when dependent on concept 22, it is dependent on concept 22g, and when dependent on concept 23, it is dependent on concept 23g), comprising a or said V_{L} domain, which V_{L} domain comprises the CDRL3 sequence of SEQ ID NO:184 or 187, or the CDRL3 sequence of SEQ ID NO:184 or 187 comprising 4 or fewer amino acid substitutions.

Concept 24h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, when dependent on concept 18, it is dependent on concept 18h, when dependent on concept 19, it is dependent on concept 19h, when dependent on concept 20, it is dependent on concept 20h, when dependent on concept 22, it is dependent on concept 22h, and when dependent on concept 23, it is dependent on concept 23h), comprising a or said V_{L} domain, which V_{L} domain comprises the CDRL3 sequence of SEQ ID NO:144 or 147, or the CDRL3 sequence of SEQ ID NO:144 or 147 comprising 4 or fewer amino acid substitutions.

Concept 24i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, when dependent on concept 18, it is dependent on concept 18i, when dependent on concept 19, it is dependent on concept 19i, when dependent on concept 20, it is dependent on concept 20i, when dependent on concept 22, it is dependent on concept 22i, and when dependent on concept 23, it is dependent on concept 23i), comprising a or said V_{L} domain, which V_{L} domain comprises the CDRL3 sequence of SEQ ID NO:250 or 253, or the CDRL3 sequence of SEQ ID NO:250 or 253 comprising 4 or fewer amino acid substitutions.

Concept 24j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, when dependent on concept 18, it is dependent on concept 18j, when dependent on concept 19, it is dependent on concept 19j, when dependent on concept 20, it is dependent on concept 20j, when dependent on concept 22, it is dependent on concept 22j, and when dependent on concept 23, it is dependent on concept 23j), comprising a or said V_{L} domain, which V_{L} domain comprises the CDRL3 sequence of SEQ ID NO:270 or 273, or the CDRL3 sequence of SEQ ID NO:270 or 273 comprising 4 or fewer amino acid substitutions.

Concept 24k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, when dependent on concept 18, it is dependent on concept 18k, when dependent on concept 19, it is dependent on concept 19k, when dependent on concept 20, it is dependent on concept 20k, when dependent on concept 22, it is dependent on concept 22k, and when dependent on concept 23, it is dependent on concept 23k), comprising a or said V_{L} domain, which V_{L} domain comprises the CDRL3 sequence of SEQ ID NO:290 or 293, or the CDRL3 sequence of SEQ ID NO:290 or 293 comprising 4 or fewer amino acid substitutions.

Concept 24l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, when dependent on concept 18, it is dependent on concept 18l, when dependent on concept 19, it is dependent on concept 19l, when dependent on concept 20, it is dependent on concept 20l, when dependent on concept 22, it is dependent on concept 22l, and when dependent on concept 23, it is dependent on concept 23l), comprising a or said V_{L} domain, which V_{L} domain comprises the CDRL3 sequence of SEQ ID NO:355 or 358, or the CDRL3 sequence of SEQ ID NO:355 or 358 comprising 4 or fewer amino acid substitutions.

Concept 25. The antibody or fragment according to any preceding concept, comprising a or said V_{L} domain, which V_{L} domain comprises an amino acid sequence of SEQ ID NO: 43, or a light chain variable domain amino acid sequence that is at least 80% (e.g. at least 85%, or at least 90%) identical to SEQ ID NO:43 (for example the V_{L} domain sequence in the light chain sequence of Seq ID No:50, 51 or 298).

Concept 25a: An antibody or a fragment thereof according to any preceding concept (but when dependent on concept 9, it is dependent on concept 9a, when dependent on concept 13, it is dependent on concept 13a, when dependent on concept 16, it is dependent on concept 16a, when dependent on concept 17, it is dependent on concept 17a, when dependent on concept 18, it is dependent on concept 18a, when dependent on concept 19, it is dependent on concept 19a, when dependent on concept 20, it is dependent on concept 20a, when dependent on concept 22, it is dependent on concept 22a, when dependent on concept 23, it is dependent on concept 23a, and when dependent on concept 24, it is dependent on concept 24a), wherein the V_{L} domain comprises an amino acid sequence of SEQ ID NO:23, or a light chain variable domain amino acid sequence that is at least 80% (e.g. at least 85%, or at least 90%) identical to SEQ ID NO:23.

Concept 25b: An antibody or a fragment thereof according to any preceding concept (but when dependent on concept 9, it is dependent on concept 9b, when dependent on concept 13, it is dependent on concept 13b, when dependent on concept 16, it is dependent on concept 16b, when dependent on concept 17, it is dependent on concept 17b, when dependent on concept 18, it is dependent on concept 18b, when dependent on concept 19, it is dependent on concept 19b, when dependent on concept 20, it is dependent on concept 20b, when dependent on concept 22, it is dependent on concept 22a, when dependent on concept 23, it is dependent on concept 23b, and when dependent on concept 24, it is dependent on concept 24b), wherein the V_{L} domain comprises an amino acid sequence of SEQ ID NO:68, or a light chain variable domain amino acid sequence that is at least 80% (e.g. at least 85%, or at least 90%) identical to SEQ ID NO:68.

Concept 25c: An antibody or a fragment thereof according to any preceding concept (but when dependent on concept 9, it is dependent on concept 9c, when dependent on concept 13, it is dependent on concept 13c, when dependent on concept 16, it is dependent on concept 16c, when dependent on concept 17, it is dependent on concept 17c, when dependent on concept 18, it is dependent on concept 18c, when dependent on concept 19, it is dependent on concept 19c, when dependent on concept 20, it is dependent on concept 20c, when dependent on concept 22, it is dependent on concept 22c, when dependent on concept 23, it is dependent on concept 23c, and when dependent on concept 24, it is dependent on concept 24c), wherein the V_{L} domain comprises an amino acid sequence of SEQ ID NO:88, or a light chain variable domain amino acid sequence that is at least 80% (e.g. at least 85%, or at least 90%) identical to SEQ ID NO:88.

Concept 25d: An antibody or a fragment thereof according to any preceding concept (but when dependent on concept 9, it is dependent on concept 9d, when dependent on concept 13, it is dependent on concept 13d, when dependent on concept 16, it is dependent on concept 16d, when dependent on concept 17, it is dependent on concept 17d, when dependent on concept 18, it is dependent on concept 18d, when dependent on concept 19, it is dependent on concept 19d, when dependent on concept 20, it is dependent on concept 20d, when dependent on concept 22, it is dependent on concept 22d, when dependent on concept 23, it is dependent on concept 23d, and when dependent on concept 24, it is dependent on concept 24d), wherein the V_{L} domain comprises an amino acid sequence of SEQ ID NO:108, or a light chain variable domain amino acid sequence that is at least 80% (e.g. at least 85%, or at least 90%) identical to SEQ ID NO:108.

Concept 25e: An antibody or a fragment thereof according to any preceding concept (but when dependent on concept 9, it is dependent on concept 9e, when dependent on concept 13, it is dependent on concept 13e, when dependent on concept 16, it is dependent on concept 16e, when dependent on concept 17, it is dependent on concept 17e, when dependent on concept 18, it is dependent on concept 18e, when dependent on concept 19, it is dependent on concept 19e, when dependent on concept 20, it is dependent on concept 20e, when dependent on concept 22, it is dependent on concept 22e, when dependent on concept 23, it is dependent on concept 23e, and when dependent on concept 24, it is dependent on concept 24e), wherein the V_{L} domain comprises an amino acid sequence of SEQ ID NO:128, or a light chain variable domain amino acid sequence that is at least 80% (e.g. at least 85%, or at least 90%) identical to SEQ ID NO:128.

Concept 25f: An antibody or a fragment thereof according to any preceding concept (but when dependent on concept 9, it is dependent on concept 9f, when dependent on concept 13, it is dependent on concept 13f, when dependent on concept 16, it is dependent on concept 16f, when dependent on concept 17, it is dependent on concept 17f, when dependent on concept 18, it is dependent on concept 18f, when dependent on concept 19, it is dependent on concept 19f, when dependent on concept 20, it is dependent on concept 20f, when dependent on concept 22, it is dependent on concept 22f, when dependent on concept 23, it is dependent on concept 23f, and when dependent on concept 24, it is dependent on concept 24f), wherein the V_{L} domain comprises an amino acid sequence of SEQ ID NO:168, or a light chain variable domain amino acid sequence that is at least 80% (e.g. at least 85%, or at least 90%) identical to SEQ ID NO:168.

Concept 25g: An antibody or a fragment thereof according to any preceding concept (but when dependent on concept 9, it is dependent on concept 9g, when dependent on concept 13, it is dependent on concept 13g, when dependent on concept 16, it is dependent on concept 16g, when dependent on concept 17, it is dependent on concept 17g, when dependent on concept 18, it is dependent on concept 18g, when dependent on concept 19, it is dependent on concept 19g, when dependent on concept 20, it is dependent on concept 20g, when dependent on concept 22, it is dependent on concept 22g, when dependent on concept 23, it is dependent on concept 23g, and when dependent on concept 24, it is dependent on concept 24g), wherein the V_{L} domain comprises an amino acid sequence of SEQ ID NO:188, or a light chain variable domain amino acid sequence that is at least 80% (e.g. at least 85%, or at least 90%) identical to SEQ ID NO:188.

Concept 25h: An antibody or a fragment thereof according to any preceding concept (but when dependent on concept 9, it is dependent on concept 9h, when dependent on concept 13, it is dependent on concept 13h, when dependent on concept 16, it is dependent on concept 16h, when dependent on concept 17, it is dependent on concept 17h, when dependent on concept 18, it is dependent on concept 18h, when dependent on concept 19, it is dependent on concept 19h, when dependent on concept 20, it is dependent on concept 20h, when dependent on concept 22, it is dependent on concept 22h, when dependent on concept 23, it is dependent on concept 23h, and when dependent on concept 24, it is dependent on concept 24h), wherein the V_{L} domain comprises an amino acid sequence of SEQ ID NO:148, or a light chain variable domain amino acid sequence that is at least 80% (e.g. at least 85%, or at least 90%) identical to SEQ ID NO:148.

Concept 25i: An antibody or a fragment thereof according to any preceding concept (but when dependent on concept 9, it is dependent on concept 9i, when dependent on concept 13, it is dependent on concept 13i, when dependent on concept 16, it is dependent on concept 16i, when dependent on concept 17, it is dependent on concept 17i, when dependent on concept 18, it is dependent on concept 18i, when dependent on concept 19, it is dependent on concept 19i, when dependent on concept 20, it is dependent on concept 20i, when dependent on concept 22, it is dependent on concept 22i, when dependent on concept 23, it is dependent on concept 23i, and when dependent on concept 24, it is dependent on concept 24i), wherein the V_{L} domain comprises an amino acid sequence of SEQ ID NO:254, or a light chain variable domain amino acid sequence that is at least 80% (e.g. at least 85%, or at least 90%) identical to SEQ ID NO:254.

Concept 25j: An antibody or a fragment thereof according to any preceding concept (but when dependent on concept 9, it is dependent on concept 9j, when dependent on concept 13, it is dependent on concept 13j, when dependent on concept 16, it is dependent on concept 16j, when dependent on concept 17, it is dependent on concept 17j, when dependent on concept 18, it is dependent on concept 18j, when dependent on concept 19, it is dependent on concept 19j, when dependent on concept 20, it is dependent on concept 20j, when dependent on concept 22, it is dependent on concept 22j, when dependent on concept 23, it is dependent on concept 23j, and when dependent on concept 24, it is dependent on concept 24j), wherein the V_{L} domain comprises an amino acid sequence of SEQ ID NO:274, or a light chain variable domain amino acid sequence that is at least 80% (e.g. at least 85%, or at least 90%) identical to SEQ ID NO:274.

Concept 25k: An antibody or a fragment thereof according to any preceding concept (but when dependent on concept 9, it is dependent on concept 9k, when dependent on concept 13, it is dependent on concept 13k, when dependent on concept 16, it is dependent on concept 16k, when dependent on concept 17, it is dependent on concept 17k, when dependent on concept 18, it is dependent on concept 18k, when dependent on concept 19, it is dependent on concept 19k, when dependent on concept 20, it is dependent on concept 20k, when dependent on concept 22, it is dependent on concept 22k, when dependent on concept 23, it is dependent on concept 23k, and when dependent on concept 24, it is dependent on concept 24k), wherein the V_{L} domain comprises an amino acid sequence of SEQ ID NO:294, or a light chain variable domain amino acid sequence that is at least 80% (e.g. at least 85%, or at least 90%) identical to SEQ ID NO:294. In one embodiment, the amino acid sequence is at least 70% identical to the specified Seq ID No. In one embodiment, the amino acid sequence is at least 75% identical to the specified Seq ID No. In one embodiment, the amino acid sequence is at least 95% identical to the specified Seq ID No. In one embodiment, the amino acid sequence is at least 96% identical to the specified Seq ID No. In one embodiment, the amino acid sequence is at least 97% identical to the specified Seq ID No. In one embodiment, the amino acid sequence is at least 98% identical to the specified Seq ID No. In one embodiment, the amino acid sequence is at least 99% identical to the specified Seq ID No. In one embodiment, the amino acid sequence is at least 99.5% identical to the specified Seq ID No.

Concept 25l: An antibody or a fragment thereof according to any preceding concept (but when dependent on concept 9, it is dependent on concept 91, when dependent on concept 13, it is dependent on concept 13l, when dependent on concept 16, it is dependent on concept 16l, when dependent on concept 17, it is dependent on concept 17l, when dependent on concept 18, it is dependent on concept 18l, when dependent on concept 19, it is dependent on concept 19l, when dependent on concept 20, it is dependent on concept 20l, when dependent on concept 22, it is dependent on concept 22l, when dependent on concept 23, it is dependent on concept 23l, and when dependent on concept 24, it is dependent on concept 24l), wherein the V_{L} domain comprises an amino acid sequence of SEQ ID NO:359, or a light chain variable domain amino acid sequence that is at least 80% (e.g. at least 85%, or at least 90%) identical to SEQ ID NO:359.

Concept 26. The antibody or fragment according to any one of concepts 12 to 21, comprising first and second copies of a or said V_{L} domain.

Concept 27. The antibody or fragment according to any preceding concept which specifically binds to cynomolgus PD-L1 as defined by Seq ID No:2. In one embodiment, the antibody or fragment binds to cynomolgus PDL-1 with an affinity of less than 1 nM (e.g. from 1 nM to 0.01 pM or from 1 nM to 0.1 pM, or from 1 nM to 1 pM). In one embodiment, the antibody or fragment binds to cynomolgus PDL-1 with an affinity of less than 10 nM (e.g. from 10 nM to 0.01 pM or from 10 nM to 0.1 pM, or from 10 nM to 1 pM). In one embodiment, the antibody or fragment binds to cynomolgus PDL-1 with an affinity of less than 0.1 nM (e.g. from 0.1 nM to 0.01 pM or from 0.1 nM to 0.1 pM, or from 0.1 nM to 1 pM). In one embodiment, the antibody or fragment binds to cynomolgus PDL-1 with an affinity of less than 0.01 nM (e.g. from 0.011 nM to 0.01 pM or from 0.01 nM to 0.1 pM). In one embodiment, the antibody or fragment binds to cynomolgus PD-L1 with an affinity of within 2-fold of the affinity to hPD-L1. In one embodiment, the antibody or fragment binds to cynomolgus PD-L1 with an affinity of within 4-fold of the affinity to hPD-L1. In one embodiment, the antibody or fragment binds to cynomolgus PD-L1 with an affinity of within 5-fold of the affinity to hPD-L1. In one embodiment, the antibody or fragment binds to cynomolgus PD-L1 with an affinity of within 6-fold of the affinity to hPD-L1. In one embodiment, the antibody or fragment binds to cynomolgus PD-L1 with an affinity of within 8-fold of the affinity to hPD-L1. In one embodiment, the antibody or fragment binds to cynomolgus PD-L1 with an affinity of within 10-fold of the affinity to hPD-L1.

In one embodiment, the antibody or fragment does not detectably bind to cynomolgus PD-L1. In one embodiment, the antibody or fragment does not detectably bind to murine PD-L1. In one embodiment, the antibody or fragment binds to murine PDL-1 with an affinity of less than 1 nM (e.g. from 1 nM to 0.01 pM or from 1 nM to 0.1 pM, or from 1 nM to 1 pM). In one embodiment, the antibody or fragment binds to murine PDL-1 with an affinity of less than 10 nM (e.g. from 10 nM to 0.01 pM or from 10 nM to 0.1 pM, or from 10 nM to 1 pM). In one embodiment, the antibody or fragment binds to murine PDL-1 with an affinity of less than 0.1 nM (e.g. from 0.1 nM to 0.01 pM or from 0.1 nM to 0.1 pM, or from 0.1 nM to 1 pM). In one embodiment, the antibody or fragment binds to murine PDL-1 with an affinity of less than 0.01 nM (e.g. from 0.011 nM to 0.01 pM or from 0.01 nM to 0.1 pM).

Concept 28. The antibody or fragment according to any preceding concept, wherein the antibody or fragment comprises a kappa light chain. Kappa light chain constant region amino acid and nucleotide sequences can be found in Seq ID Nos: 206 to 215. In one embodiment, the light chain may be a lambda light chain. Lambda light chain constant region amino acid and nucleotide sequences can be found in Seq ID Nos: 216 to 237 and Seq ID No: 535, Seq ID No:536 and Seq ID No:538.

Concept 29. The antibody or fragment according to any one of concepts 9 to 28, wherein the amino acid substitutions are conservative amino acid substitutions, optionally wherein the conservative substitutions are from one of six groups (each group containing amino acids that are conservative substitutions for one another) selected from:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Conservative substitutions may be as described above in concept 9.

Concept 30. The antibody or fragment according to any preceding concept, wherein the antibody or fragment comprises a constant region, such as a human constant region, for example an effector-null human constant region, e.g. an IgG4 constant region or an IgG1 constant region, optionally wherein the constant region is IgG4-PE (Seq ID No:199), or a disabled IgG1 as defined in Seq ID No:205. In other embodiments, the antibody or fragment is any of the isotypes or constant regions as defined hereinabove. In one embodiment, the constant region is wild-type human IgG1 (Seq ID No:340). For example, the constant region is an effector-enabled IgG1 constant region, optionally having ADCC and/or CDC activity. In one embodiment, the constant region is engineered for enhanced ADCC and/or CDC and/or ADCP. In another embodiment, the constant region is engineered for enhanced effector function.

The IgG4 constant region may be any of the IgG4 constant region amino acid sequences, or encoded by any of the nucleic acid sequences of Seq ID Nos: 192 to 203. A heavy chain constant region may be an IgG4 comprising both the Leu235Glu mutation and the Ser228Pro mutation. This “IgG4-PE” heavy chain constant region (Seq ID Nos: 198, encoded by Seq ID Nos: 199, 200 and 201) is effector null.

An alternative effector null human constant region is a disabled IgG1 being an IgG1*01 allele comprising the L235A and/or G237A mutations (e.g. LAGA, Seq ID No:204, encoded by Seq ID No:205). In one embodiment, the antibodies or antibody fragments disclosed herein comprise an IgG1 heavy chain constant region, wherein the sequence contains alanine at position 235 and/or 237 (EU index numbering).

The antibody-dependent cell phagocytosis (ADCP) mechanism is discussed in Gül et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015.

The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues (e.g. as described in Lazar et al., 2006, Proc. Natl. Acad. Sci. U.S.A., March 14; 103(11):4005-10; the modifications disclosed therein are incorporated herein by reference). Human IgG1 constant regions containing specific mutations or altered glycosylation on residue Asn297 (e.g. N297Q, EU index numbering) have been shown to enhance binding to Fc receptors. In one embodiment, such mutations are one or more of the residues selected from 239, 332 and 330 for human IgG1 constant regions (or the equivalent positions in other IgG isotypes). In one embodiment, the antibody or fragment comprises a human IgG1 constant region having one or more mutations independently selected from N297Q, S239D, 1332E and A330L (EU index numbering).

In another embodiment, the increase in affinity for Fc-receptors is achieved by altering the natural glycosylation profile of the Fc domain by, for example, generating under fucosylated or de-fucosylated variants (as described in Natsume et al., 2009, Drug Des. Devel. Ther., 3:7-16 or by Zhou Q., Biotechnol. Bioeng., 2008 Feb. 15, 99(3):652-65), the modifications described therein are incorporated herein by reference). Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. For example, to increase ADCC, residues in the hinge region can be altered to increase binding to Fc-gamma RIII (see, for example, Shields et al., 2001, J. Biol. Chem., March 2; 276(9):6591-604; the modifications described therein are incorporated herein by reference). Thus, in one embodiment, the antibody or fragment comprises a human IgG heavy chain constant region that is a variant of a wild-type human IgG heavy chain constant region, wherein the variant human IgG heavy chain constant region binds to human Fcγ receptors selected from the group consisting of FcγRIIB and FcγRIIA with higher affinity than the wild type human IgG heavy chain constant region binds to the human Fcγ receptors. In one embodiment, the antibody or fragment comprises a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. In one embodiment, the variant human IgG heavy chain constant region is a variant human IgG1, a variant human IgG2, or a variant human IgG4 heavy chain constant region. In one embodiment, the variant human IgG heavy chain constant region comprises one or more amino acid mutations selected from G236D, P238D, S239D, S267E, L328F, and L328E (EU index numbering system). In another embodiment, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). In another embodiment, the variant human IgG heavy chain constant region further comprises one or more amino acid mutations that reduce the affinity of the IgG for human FcγRIIIA, human FcγRIIA, or human FcγRI. In one embodiments, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V3051, A330L, 1332E, E333A, K334A, A339T, and P396L (EU index numbering system). In one embodiment, the variant human IgG heavy chain constant region comprises a set of amino acid mutations selected from the group consisting of: S239D; T256A; K290A; S298A; 1332E; E333A; K334A; A339T; S239D and 1332E; S239D, A330L, and 1332E; S298A, E333A, and K334A; G236A, S239D, and 1332E; and F243L, R292P, Y300L, V3051, and P396L (EU index numbering system). In one embodiment, the variant human IgG heavy chain constant region comprises a S239D, A330L, or 1332E amino acid mutations (EU index numbering system). In one embodiment, the variant human IgG heavy chain constant region comprises an S239D and 1332E amino acid mutations (EU index numbering system). In one embodiment, the variant human IgG heavy chain constant region is a variant human IgG1 heavy chain constant region comprising the S239D and 1332E amino acid mutations (EU index numbering system). In one embodiment, the antibody or fragment comprises an afucosylated Fc region. In another embodiment, the antibody or fragment thereof is defucosylated. In another embodiment, the antibody or fragment is under fucosylated.

In another embodiment, the antibodies and fragments disclosed herein may comprise a triple mutation (M252Y/S254T/T256E) which enhances binding to FcRn. See Dall et al., Immunol 2002; 169:5171-5180 for a discussion of mutations affection FcRn binding in table 2, the mutations described therein are incorporated herein by reference.

Equally, the enhancement of CDC may be achieved by amino acid changes that increase affinity for C1q, the first component of the classic complement activation cascade (see Idusogie et al., J. Immunol., 2001, 166:2571-2575; the modifications described are incorporated herein by reference). Another approach is to create a chimeric Fc domain created from human IgG1 and human IgG3 segments that exploit the higher affinity if IgG3 for C1q (Natsume et al., 2008, Cancer Res., 68:3863-3872; the modifications are incorporated herein by reference). In another embodiment, the antibody or antibody fragments disclosed herein may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a constant region comprising one or more mutations selected from E345K, E430G, R344D and D356R, in particular a double mutation comprising R344D and D356R (EU index numbering system).

An antibody may have a heavy chain constant region that binds one or more types of Fc receptor but does not induce cellular effector functions, i.e. which does not mediate ADCC, CDC or ADCP activity. Such a constant region may be unable to bind the particular Fc receptor(s) responsible for triggering ADCC, CDC or ADCP activity. An antibody may have a heavy chain constant region that does not bind Fcγ receptors. Thus, in one embodiment, the constant region may comprise a Leu235Glu mutation (EU index numbering system).

In another embodiment, the antibodies and fragments disclosed herein are modified to increase or decrease serum half-life. In one embodiment, one or more of the following mutations: T252L, T254S or T256F are introduced to increase biological half-life of the antibody. Biological half-life can also be increased by altering the heavy chain constant region CH₁ domain or CL region to contain a salvage receptor binding epitope taken from two loops of a CH₂ domain of an Fc region of an IgG, as described in U.S. Pat. Nos. 5,869,046 and 6,121,022, the modifications described therein are incorporated herein by reference. In another embodiment, the Fc hinge region of an antibody or antigen-binding fragment of the invention is mutated to decrease the biological half-life of the antibody or fragment. One or more amino acid mutations are introduced into the CH₂-CH₃ domain interface region of the Fc-hinge fragment such that the antibody or fragment has impaired Staphylococcyl protein A (SpA) binding relative to native Fc-hinge domain SpA binding. Other methods of increasing serum half-life are known to those skilled in the art. Thus, in one embodiment, the antibody or fragment is PEGylated. In another embodiment, the antibody or fragment is fused to an albumin-binding domain, e.g. an albumin binding single domain antibody (dAb). In another embodiment, the antibody or fragment is PASylated (i.e. genetic fusion of polypeptide sequences composed of PAS (XL-Protein GmbH) which forms uncharged random coil structures with large hydrodynamic volume). In another embodiment, the antibody or fragment is XTENylated®/rPEGylated (i.e. genetic fusion of non-exact repeat peptide sequence (Amunix, Versartis) to the therapeutic peptide). In another embodiment, the antibody or fragment is ELPylated (i.e. genetic fusion to ELP repeat sequence (PhaseBio)). These various half-life extending fusions are described in more detail in Strohl, BioDrugs (2015) 29:215-239, which fusions, e.g. in Tables 2 and 6, are incorporated herein by reference.

The antibody may have a modified constant region which increases stability. Thus, in one embodiment, the heavy chain constant region comprises a Ser228Pro mutation. In another embodiment, the antibodies and fragments disclosed herein comprise a heavy chain hinge region that has been modified to alter the number of cysteine residues. This modification can be used to facilitate assembly of the light and heavy chains or to increase or decrease the stability of the antibody.

Concept 31. The antibody or fragment according to concept 30, wherein the constant region is a murine constant region. In other embodiments, the constant region may be of any non-human mammalian origin, e.g. rat, mouse, hamster, guinea pig, dog, cat, horse, chicken, llama, dromedary, etc. In one embodiment, the constant region is a rat constant region. In another embodiment, the constant region is a llama constant region. The murine constant region may be any of the isotypes or alleles described hereinabove.

Concept 32. The antibody or fragment according to concept 30 or concept 31, wherein the constant region has CDC and/or ADCC activity.

Concept 33. The antibody according to any preceding concept wherein the:
a) V_{H} domain comprises an amino acid sequence of SEQ ID No:33 and the V_{L} domain comprises an amino acid sequence of SEQ ID No:43;
b) V_{H} domain comprises an amino acid sequence that is at least 85% identical to SEQ ID No:33, and the V_{L} domain comprises an amino acid sequence that is at least 85% identical to SEQ ID No:43;
c) V_{H} domain comprises an amino acid sequence of the V_{H} domain of SEQ ID No: 47 and the V_{L} domain comprises an amino acid sequence of SEQ ID No:43;
d) V_{H} domain comprises an amino acid sequence of the V_{H} domain of SEQ ID No: 48 and the V_{L} domain comprises an amino acid sequence of SEQ ID No:43;
e) V_{H} domain comprises an amino acid sequence of the V_{H} domain of SEQ ID No: 49 and the V_{L} domain comprises an amino acid sequence of SEQ ID No:43;
f) V_{H} domain comprises an amino acid sequence of the V_{H} domain of SEQ ID No: 342 and the V_{L} domain comprises an amino acid sequence of SEQ ID No: 43;
g) V_{H} domain comprises an amino acid sequence of SEQ ID No:33 and the V_{L} domain comprises an amino acid sequence of the V_{L} domain of SEQ ID No:50;
h) V_{H} domain comprises an amino acid sequence of the V_{H} domain of SEQ ID No: 47 and the V_{L} domain comprises an amino acid sequence of the V_{L} domain of SEQ ID No:50;
i) V_{H} domain comprises an amino acid sequence of the V_{H} domain of SEQ ID No: 48 and the V_{L} domain comprises an amino acid sequence of the V_{L} domain of SEQ ID No:50;
j) V_{H} domain comprises an amino acid sequence of the V_{H} domain of SEQ ID No: 49 and the V_{L} domain comprises an amino acid sequence of the V_{L} domain of SEQ ID No:50;
k) V_{H} domain comprises an amino acid sequence of the V_{H} domain of SEQ ID No: 342 and the V_{L} domain comprises an amino acid sequence of the V_{L} domain of SEQ ID No:50;
l) V_{H} domain comprises an amino acid sequence of SEQ ID No:33 and the V_{L} domain comprises an amino acid sequence of the V_{L} domain of SEQ ID No:51;
m) V_{H} domain comprises an amino acid sequence of the V_{H} domain of SEQ ID No: 47 and the V_{L} domain comprises an amino acid sequence of the V_{L} domain of SEQ ID No:51;
n) V_{H} domain comprises an amino acid sequence of the V_{H} domain of SEQ ID No: 48 and the V_{L} domain comprises an amino acid sequence of the V_{L} domain of SEQ ID No:51;
o) V_{H} domain comprise an amino acid sequence of the V_{H} domain of SEQ ID No:49 and the V_{L} domain comprises an amino acid sequence of the V_{L} domain of SEQ ID No: 51;
p) V_{H} domain comprise an amino acid sequence of the V_{H} domain of SEQ ID No: 342 and the V_{L} domain comprises an amino acid sequence of the V_{L} domain of SEQ ID No:51;
q) V_{H} domain comprises an amino acid sequence of SEQ ID No:33 and the V_{L} domain comprises an amino acid sequence of the V_{L} domain of SEQ ID No:298;
r) V_{H} domain comprises an amino acid sequence of the V_{H} domain of SEQ ID No: 47 and the V_{L} domain comprises an amino acid sequence of the V_{L} domain of SEQ ID No:298;
s) V_{H} domain comprises an amino acid sequence of the V_{H} domain of SEQ ID No: 48 and the V_{L} domain comprises an amino acid sequence of the V_{L} domain of SEQ ID No:298;
t) V_{H} domain comprise an amino acid sequence of the V_{H} domain of SEQ ID No:49 and the V_{L} domain comprises an amino acid sequence of the V_{L} domain of SEQ ID No: 298;
u) V_{H} domain comprise an amino acid sequence of the V_{H} domain of SEQ ID No: 342 and the V_{L} domain comprises an amino acid sequence of the V_{L} domain of SEQ ID No:298;
v) V_{H} domain comprises an amino acid sequence of SEQ ID No:58 and the V_{L} domain comprises an amino acid sequence of SEQ ID No:68;
w) V_{H} domain comprises an amino acid sequence that is at least 85% identical to SEQ ID No:58, and the V_{L} domain comprise an amino acid sequence that is at least 85% identical to SEQ ID No:68;
x) V_{H} domain comprises an amino acid sequence of SEQ ID No:78 and the V_{L} domain comprises an amino acid sequence of SEQ ID No:88;
y) V_{H} domain comprises an amino acid sequence that is at least 85% identical to SEQ ID No:78, and the V_{L} domain comprises an amino acid sequence that is at least 85% identical to SEQ ID No:88;
z) V_{H} domain comprises an amino acid sequence of SEQ ID No:98 and the V_{L} domain comprises an amino acid sequence of SEQ ID No:108;
aa) V_{H} domain comprises an amino acid sequence that is at least 85% identical to SEQ ID No:98, and the V_{L} domain comprises an amino acid sequence that is at least 85% identical to SEQ ID No:108;
bb) V_{H} domain comprises an amino acid sequence of SEQ ID No:118 and the V_{L} domain comprises an amino acid sequence of SEQ ID No:128;
cc) V_{H} domain comprises an amino acid sequence that is at least 85% identical to SEQ ID No:118, and the V_{L} domain comprises an amino acid sequence that is at least 85% identical to SEQ ID No:128;
dd) V_{H} domain comprises an amino acid sequence of SEQ ID No:158 and the V_{L} domain comprises an amino acid sequence of SEQ ID No:168;
ee) V_{H} domain comprises an amino acid sequence that is at least 85% identical to SEQ ID No:158, and the V_{L} domain comprises an amino acid sequence that is at least 85% identical to SEQ ID No:168;
ff) V_{H} domain comprises an amino acid sequence of SEQ ID No:178 and the V_{L} domain comprises an amino acid sequence of SEQ ID No:188;
gg) V_{H} domain comprises an amino acid sequence that is at least 85% identical to SEQ ID No:178, and the V_{L} domain comprises an amino acid sequence that is at least 85% identical to SEQ ID No:188;
hh) V_{H} domain comprises an amino acid sequence of SEQ ID No:138 and the V_{L} domain comprises an amino acid sequence of SEQ ID No:148;
ii) V_{H} domain comprises an amino acid sequence that is at least 85% identical to SEQ ID No:138 and the V_{L} domain comprises an amino acid sequence that is at least 85% identical to SEQ ID No:148;
jj) V_{H} domain comprises an amino acid sequence of SEQ ID No:244 and the V_{L} domain comprises an amino acid sequence of SEQ ID No:254;
kk) V_{H} domain comprises an amino acid sequence that is at least 85% identical to SEQ ID No:244, and the V_{L} domain comprises an amino acid sequence that is at least 85% identical to SEQ ID No:254;
ll) V_{H} domain comprises an amino acid sequence of SEQ ID No:264 and the V_{L} domain comprises an amino acid sequence of SEQ ID No:274;
mm) V_{H} domain comprises an amino acid sequence that is at least 85% identical to SEQ ID No:264, and the V_{L} domain comprises an amino acid sequence that is at least 85% identical to SEQ ID No:274;
nn) V_{H} domain comprises an amino acid sequence of SEQ ID No:284 and the V_{L} domain comprises an amino acid sequence of SEQ ID No:294; and
oo) V_{H} domain comprises an amino acid sequence that is at least 85% identical to SEQ ID No:284, and the V_{L} domain comprises an amino acid sequence that is at least 85% identical to SEQ ID No:294;
pp) V_{H} domain comprises an amino acid sequence of SEQ ID No:349 and the V_{L} domain comprises an amino acid sequence of SEQ ID No:359; and
qq) V_{H} domain comprises an amino acid sequence that is at least 85% identical to SEQ ID No:349, and the V_{L} domain comprises an amino acid sequence that is at least 85% identical to SEQ ID No:359.

In one embodiment, the amino acid sequence is at least 70% identical to the specified Seq ID No. In one embodiment, the amino acid sequence is at least 75% identical to the specified Seq ID No. In one embodiment, the amino acid sequence is at least 95% identical to the specified Seq ID No. In one embodiment, the amino acid sequence is at least 96% identical to the specified Seq ID No. In one embodiment, the amino acid sequence is at least 97% identical to the specified Seq ID No. In one embodiment, the amino acid sequence is at least 98% identical to the specified Seq ID No. In one embodiment, the amino acid sequence is at least 99% identical to the specified Seq ID No. In one embodiment, the amino acid sequence is at least 99.5% identical to the specified Seq ID No.

Concept 34. The antibody according to any preceding concept wherein the antibody comprises a heavy chain and a light chain, and
a) the heavy chain amino acid sequence comprises an amino acid sequence of SEQ ID No: 35 and the light chain amino acid sequence comprises an amino acid sequence of SEQ ID No:45;
b) the heavy chain amino acid sequence comprises an amino acid sequence that is at least 85% identical to SEQ ID No:35 and the light chain amino acid sequence comprises an amino acid sequence that is at least 85% identical to SEQ ID No:45;
c) the heavy chain amino acid sequence comprises an amino acid sequence of SEQ ID No: 47 and the light chain amino acid sequence comprises an amino acid sequence of SEQ ID No:45;
d) the heavy chain amino acid sequence comprises an amino acid sequence of SEQ ID No: 48 and the light chain amino acid sequence comprises an amino acid sequence of SEQ ID No:45;
e) the heavy chain amino acid sequence comprises an amino acid sequence of SEQ ID No: 49 and the light chain amino acid sequence comprises an amino acid sequence of SEQ ID No:45;
f) the heavy chain amino acid sequence comprises an amino acid sequence of SEQ ID No: 342 and the light chain amino acid sequence comprises an amino acid sequence of SEQ ID No:45;
g) the heavy chain amino acid sequence comprises an amino acid sequence of SEQ ID No: 35 and the light chain amino acid sequence comprises an amino acid sequence of SEQ ID No:50;
h) the heavy chain amino acid sequence comprises an amino acid sequence of SEQ ID No: 47 and the light chain amino acid sequence comprises an amino acid sequence of SEQ ID No:50;
i) the heavy chain amino acid sequence comprises an amino acid sequence of SEQ ID No: 48 and the light chain amino acid sequence comprises an amino acid sequence of SEQ ID No:50;
j) the heavy chain amino acid sequence comprises an amino acid sequence of SEQ ID No: 49 and the light chain amino acid sequence comprises an amino acid sequence of SEQ ID No:50;
k) the heavy chain amino acid sequence comprises an amino acid sequence of SEQ ID No: 342 and the light chain amino acid sequence comprises an amino acid sequence of SEQ ID No:50;
l) the heavy chain amino acid sequence comprises an amino acid sequence of SEQ ID No: 35 and the light chain amino acid sequence comprises an amino acid sequence of SEQ ID No:51;
m) the heavy chain amino acid sequence comprises an amino acid sequence of SEQ ID No: 47 and the light chain amino acid sequence comprises an amino acid sequence of SEQ ID No:51;
n) the heavy chain amino acid sequence comprises an amino acid sequence of SEQ ID No: 48 and the light chain amino acid sequence comprises an amino acid sequence of SEQ ID No:51;
o) the heavy chain amino acid sequence comprises an amino acid sequence of SEQ ID No: 49 and the light chain amino acid sequence comprises an amino acid sequence of SEQ ID No:51;
p) the heavy chain amino acid sequence comprises an amino acid sequence of SEQ ID No: 342 and the light chain amino acid sequence comprises an amino acid sequence of SEQ ID No:51;
q) the heavy chain amino acid sequence comprises an amino acid sequence of SEQ ID No: 35 and the light chain amino acid sequence comprises an amino acid sequence of SEQ ID No:298;
r) the heavy chain amino acid sequence comprises an amino acid sequence of SEQ ID No: 47 and the light chain amino acid sequence comprises an amino acid sequence of SEQ ID No:298;
s) the heavy chain amino acid sequence comprises an amino acid sequence of SEQ ID No: 48 and the light chain amino acid sequence comprises an amino acid sequence of SEQ ID No:298;
t) the heavy chain amino acid sequence comprises an amino acid sequence of SEQ ID No: 49 and the light chain amino acid sequence comprises an amino acid sequence of SEQ ID No:298;
u) the heavy chain amino acid sequence comprises an amino acid sequence of SEQ ID No: 342 and the light chain amino acid sequence comprises an amino acid sequence of SEQ ID No:298;
v) the heavy chain amino acid sequence comprises an amino acid sequence of SEQ ID No: 60 and the light chain amino acid sequence comprises an amino acid sequence of SEQ ID No:70;
w) the heavy chain amino acid sequence comprises an amino acid sequence that is at least 85% identical to SEQ ID No:60, and the light chain amino acid sequence comprises an amino acid sequence that is at least 85% identical to SEQ ID No:70;
x) the heavy chain amino acid sequence comprises an amino acid sequence of SEQ ID No: 80 and the light chain amino acid sequence comprises an amino acid sequence of SEQ ID No:90;
y) the heavy chain amino acid sequence comprises an amino acid sequence that is at least 85% identical to SEQ ID No:80, and the light chain amino acid sequence comprises an amino acid sequence that is at least 85% identical to SEQ ID No:90;
z) the heavy chain amino acid sequence comprises an amino acid sequence of SEQ ID No: 100 and the light chain amino acid sequence comprises an amino acid sequence of SEQ ID No:110;
aa) the heavy chain amino acid sequence comprises an amino acid sequence that is at least 85% identical to SEQ ID No:100, and the light chain amino acid sequence comprises an amino acid sequence that is at least 85% identical to SEQ ID No: 110;
bb) the heavy chain amino acid sequence comprises an amino acid sequence of SEQ ID No: 120 and the light chain amino acid sequence comprises an amino acid sequence of SEQ ID No:130;
cc) the heavy chain amino acid sequence comprises an amino acid sequence that is at least 85% identical to SEQ ID No:120, and the light chain amino acid sequence comprises an amino acid sequence that is at least 85% identical to SEQ ID No: 130;
dd) the heavy chain amino acid sequence comprises an amino acid sequence of SEQ ID No: 160 and the light chain amino acid sequence comprises an amino acid sequence of SEQ ID No:170;
ee) the heavy chain amino acid sequence comprises an amino acid sequence that is at least 85% identical to SEQ ID No:160, and the light chain amino acid sequence comprises an amino acid sequence that is at least 85% identical to SEQ ID No: 170;
ff) the heavy chain amino acid sequence comprises an amino acid sequence of SEQ ID No: 180 and the light chain amino acid sequence comprises an amino acid sequence of SEQ ID No:190;
gg) the heavy chain amino acid sequence comprises an amino acid sequence that is at least 85% identical to SEQ ID No:180, and the light chain amino acid sequence comprises an amino acid sequence that is at least 85% identical to SEQ ID No: 190
hh) the heavy chain amino acid sequence comprises an amino acid sequence of SEQ ID No: 140 and the light chain amino acid sequence comprises an amino acid sequence of SEQ ID No:150;
ii) the heavy chain amino acid sequence comprises an amino acid sequence that is at least 85% identical to SEQ ID No:140, and the light chain amino acid sequence comprises an amino acid sequence that is at least 85% identical to SEQ ID No: 150;
jj) the heavy chain amino acid sequence comprises an amino acid sequence of SEQ ID No: 246 and the light chain amino acid sequence comprises an amino acid sequence of SEQ ID No:256;
kk) the heavy chain amino acid sequence comprises an amino acid sequence that is at least 85% identical to SEQ ID No:246, and the light chain amino acid sequence comprises an amino acid sequence that is at least 85% identical to SEQ ID No: 256;
ll) the heavy chain amino acid sequence comprises an amino acid sequence of SEQ ID No: 266 and the light chain amino acid sequence comprises an amino acid sequence of SEQ ID No:276;
mm) the heavy chain amino acid sequence comprises an amino acid sequence that is at least 85% identical to SEQ ID No:266, and the light chain amino acid sequence comprises an amino acid sequence that is at least 85% identical to SEQ ID No:276;
nn) the heavy chain amino acid sequence comprises an amino acid sequence of SEQ ID No: 286 and the light chain amino acid sequence comprises an amino acid sequence of SEQ ID No:296; and
oo) the heavy chain amino acid sequence comprises an amino acid sequence that is at least 85% identical to SEQ ID No:286, and the light chain amino acid sequence comprises an amino acid sequence that is at least 85% identical to SEQ ID No: 296;
pp) the heavy chain amino acid sequence comprises an amino acid sequence of SEQ ID No:351 and the light chain amino acid sequence comprises an amino acid sequence of SEQ ID No:361; and
qq) the heavy chain amino acid sequence comprises an amino acid sequence that is at least 85% identical to SEQ ID No:351, and the light chain amino acid sequence comprises an amino acid sequence that is at least 85% identical to SEQ ID No:361.

In one embodiment, the amino acid sequence is at least 70% identical to the specified Seq ID No. In one embodiment, the amino acid sequence is at least 75% identical to the specified Seq ID No. In one embodiment, the amino acid sequence is at least 95% identical to the specified Seq ID No. In one embodiment, the amino acid sequence is at least 96% identical to the specified Seq ID No. In one embodiment, the amino acid sequence is at least 97% identical to the specified Seq ID No. In one embodiment, the amino acid sequence is at least 98% identical to the specified Seq ID No. In one embodiment, the amino acid sequence is at least 99% identical to the specified Seq ID No. In one embodiment, the amino acid sequence is at least 99.5% identical to the specified Seq ID No.

Concept 35. The antibody or fragment according to any preceding concept which competes for binding to hPD-L1 with the antibody 1D05, optionally wherein the competition for binding to hPDL-1 is conducted using SPR. SPR may be carried out as described hereinabove, or as described in concept 16.

Concept 36. The antibody or fragment according to any preceding concept wherein the antibody or fragment is capable of inhibiting PD-L1-mediated suppression of T-cells, optionally wherein the suppression of T-cells is measured by an increase in one or more of IFNγ, IL-2, CD25 or proliferation of T-cells in an assay that provides co-stimulation by either direct CD3/CD28 stimulation, superantigen stimulation or provides co-stimulation by co-incubation with cells capable of inducing a T-cell response. The measurements may be carried out with any suitable technique. For example, the measurements may be taken with ELISA, HTRF, BRDU incorporation (proliferation), electrochemiluminescence (ECL) or flow cytometry (e.g. FACS). These techniques are well-known to those skilled in the art and are described elsewhere herein. In one embodiment, the assay is flow cytometry. In one embodiment, the assay is ELISA. In one embodiment, the assay is HTRF. In one embodiment, the suppression of T-cells is measured by an increase in IFNγ. In one embodiment, the suppression of T-cells is measured by an increase in IL-2. In one embodiment, the suppression of T-cells is measured by an increase in CD25. In one embodiment, the suppression of T-cells is measured by an increase in IFNγ and IL-2. In one embodiment, the suppression of T-cells is measured by an increase in IFNγ and CD25. In one embodiment, the suppression of T-cells is measured by an increase in CD25 and IL-2. In one embodiment, the suppression of T-cells is measured by an increase in IFNγ, IL-2 and CD25. In one embodiment, the co-stimulation is provided by direct CD3/CD28 stimulation. In one embodiment, the co-stimulation is provided by a superantigen, such as staphylococcal enterotoxin B (SEB). In one embodiment, the assay provides co-stimulation by co-incubation with cells capable of inducing a T-cell response. Such cells may be antigen-presenting cells (APCs), for example monocytes, B-cells or dendritic cells. In one embodiment, the assay provides co-stimulation by co-incubation with APCs. In one embodiment, the assay provides co-stimulation by co-incubation with monocytes. In one embodiment, the assay provides co-stimulation by co-incubation with B-cells. In one embodiment, the assay provides co-stimulation by co-incubation with dendritic cells.

Concept 37. A bispecific antibody or fusion protein comprising an antibody or fragment thereof as defined in any preceding concept.

Concept 37a. A dual binding antibody or fusion protein comprising an antibody or fragment thereof as defined in any preceding concept. A dual binding antibody has the meaning as set out above.

Concept 38. The bispecific antibody according to concept 37, wherein the bispecific format is selected from DVD-Ig, mAb², FIT-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, minibody, knobs-in-holes, knobs-in-holes with common light chain, knobs-in-holes with common light chain and charge pairs, charge pairs, charge pairs with common light chain, in particular mAb², knob-in-holes, knob-in-holes with common light chain, knobs-in-holes with common light chain and charge pairs and FIT-Ig, e.g. mAb² and FIT-Ig.

In one embodiment, the bispecific format is selected from DVD-Ig, mAb², FIT-Ig, mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody.

In one embodiment, the bispecific format is selected from DVD-Ig, FIT-Ig, mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, FIT-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, minibody, knobs-in-holes, knobs-in-holes with common light chain, knobs-in-holes with common light chain and charge pairs, charge pairs, charge pairs with common light chain, in particular knob-in-holes, knob-in-holes with common light chain, knobs-in-holes with common light chain and charge pairs and FIT-Ig, e.g. FIT-Ig.

In one embodiment, the bispecific format is selected from DVD-Ig, mAb², mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, mAb², mAb-dAb, dock and lock, SEEDbody, scDiabody-Fc, diabody-Fc, tandem scFv-Fc, Fab-scFv-Fc, Fab-scFv, intrabody, BITE, diabody, DART, TandAb, scDiabody, scDiabody-CH3, Diabody-CH3, minibody, knobs-in-holes, knobs-in-holes with common light chain, knobs-in-holes with common light chain and charge pairs, charge pairs, charge pairs with common light chain, in particular mAb², knob-in-holes, knobs-in-holes with common light chain and charge pairs, and knob-in-holes with common light chain, e.g. mAb².

In one embodiment, the bispecific format is selected from DVD-Ig, mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)₂-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody, for example DVD-Ig, mAb-dAb, dock and lock, SEEDbody, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, minibody, knobs-in-holes, knobs-in-holes with common light chain, knobs-in-holes with common light chain and charge pairs, charge pairs, charge pairs with common light chain, in particular knob-in-holes, knobs-in-holes with common light chain and charge pairs, and knob-in-holes with common light chain.

Concept 39. The bispecific antibody according to concept 37 or concept 38, wherein the bispecific antibody specifically binds to hPD-L1 and another target antigen selected from immune checkpoint inhibitors (such as PD-1, CTLA-4, TIGIT, TIM-3, LAG-3 and VISTA, e.g. TIGIT, TIM-3 and LAG-3), immune modulators (such as BTLA, hHVEM, CSF1R, CCR4, CD39, CD40, CD73, CD96, CXCR2, CXCR4, CD200, GARP, SIRPα, CXCL9, CXCL10, CXCL11 and CD155, e.g. GARP, SIRPα, CXCR4, BTLA, hVEM and CSF1R), immune activators (such as CD137, GITR, OX40, CD40, CXCR3 (e.g. agonistic anti-CXCR3 antibodies), CD27, CD3, ICOS (e.g. agonistic anti-ICOS antibodies), for example ICOS, CD137, GITR and OX40).

Concept 39a. A bispecific antibody which binds to hPD-L1 with a V_{H}, a V_{L}, or a paired V_{H} and V_{L} comprising one or more of the CDRs (e.g. CDRH3 and CDRL3) or variable region sequences of any of the antibodies described in Aspect 1a hereinbelow, and another target antigen selected from immune checkpoint inhibitors (such as PD-1, CTLA-4, TIGIT, TIM-3, LAG-3 and VISTA, e.g. TIGIT, TIM-3 and LAG-3), immune modulators (such as BTLA, hHVEM, CSF1R, CCR4, CD39, CD40, CD73, CD96, CXCR2, CXCR4, CD200, GARP, SIRPα, CXCL9, CXCL10, CXCL11 and CD155, e.g. GARP, SIRPα, CXCR4, BTLA, hVEM and CSF1R), immune activators (such as CD137, GITR, OX40, CD40, CXCR3 (e.g. agonistic anti-CXCR3 antibodies), CD27, CD3, ICOS (e.g. agonistic anti-ICOS antibodies), for example ICOS, CD137, GITR and OX40).

Concept 39b. The bispecific antibody according to concept 37 or concept 38, wherein the bispecific antibody specifically binds to hPD-L1 and another target antigen selected from immune checkpoint inhibitors (such as PD-1, CTLA-4, TIGIT, TIM-3, LAG-3 and VISTA, e.g. TIGIT, TIM-3 and LAG-3), immune modulators (such as BTLA, hHVEM, CSF1R, CCR4, CD39, CD40, CD73, CD96, CXCR2, CXCR4, CD200, GARP, SIRPα, CXCL9, CXCL10, CXCL11 and CD155, e.g. GARP, SIRPα, CXCR4, BTLA, hVEM and CSF1R), immune activators (such as CD137, GITR, OX40, CD40, CXCR3 (e.g. agonistic anti-CXCR3 antibodies), CD3, ICOS (e.g. agonistic anti-ICOS antibodies), for example ICOS, CD137, GITR and OX40).

In one embodiment, the another target antigen is an immune checkpoint inhibitor, such as PD-1, CTLA-4, TIGIT, TIM-3, LAG-3 and VISTA, e.g. TIGIT, CTLA-4, TIM-3 and LAG-3. In one embodiment, the another target antigen is an immune modulator, such as BTLA, hHVEM, CSF1R, CCR4, CD39, CD40, CD73, CD96, CXCR2, CXCR4, CD200, GARP, SIRPα, CXCL9, CXCL10, CXCL11 and CD155, e.g. GARP, SIRPα, CXCR4, BTLA, hVEM and CSF1R. In one embodiment, the another target antigen is an immune activator, such as CD137, GITR, OX40, CD40, CXCR3 (e.g. agonistic anti-CXCR3 antibodies), CD3, CD27 and ICOS (e.g. agonistic anti-ICOS antibodies), or CD137, GITR, OX40, CD40, CXCR3 (e.g. agonistic anti-CXCR3 antibodies), CD3 and ICOS (e.g. agonistic anti-ICOS antibodies), for example ICOS, CD137, GITR and OX40).

In one embodiment, the another target antigen is CTLA-4. In one embodiment, the another target antigen is TIGIT. In one embodiment, the another target antigen is TIM-3. In one embodiment, the another target antigen is LAG-3. In one embodiment, the another target antigen is GITR. In one embodiment, the another target antigen is VISTA. In one embodiment, the another target antigen is CD137. In one embodiment, the another target antigen is SIRPα. In one embodiment, the another target antigen is CXCL10. In one embodiment, the another target antigen is CD155. In one embodiment, the another target antigen is CD40.

In another embodiment, the bispecific antibody binds another target antigen which is PD-1 and the binding to PD-1 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CTLA4 and the binding to CTLA4 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is TIGIT and the binding to TIGIT is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is TIM-3 and the binding to TIM-3 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is LAG3 and the binding to LAG3 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is VISTA and the binding to VISTA is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is BTLA and the binding to BTLA is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is hHVEM and the binding to hHVEM is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CSF1R and the binding to CSF1R is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CCR4 and the binding to CCR4 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD39 and the binding to CD39 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD40 and the binding to CD40 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD73 and the binding to CD73 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD96 and the binding to CD96 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCR2 and the binding to CXCR2 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCR4 and the binding to CXCR4 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD200 and the binding to CD200 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is GARP and the binding to GARP is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is SIRPα and the binding to SIRPα is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCL9 and the binding to CXCL9 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCL10 and the binding to CXCL10 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCL11 and the binding to CXCL11 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD155 and the binding to CD155 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD137 and the binding to CD137 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is GITR and the binding to GITR is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is OX40 and the binding to OX40 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD40 and the binding to CD40 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CXCR3 and the binding to CXCR3 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD27 and the binding to CD27 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is CD3 and the binding to CD3 is provided by an antigen-binding domain (for example, a V_{H}, a V_{L} or a paired V_{H} and V_{L}) having any of the sequences, including CDR sequences (for example CDRH3 and/or CDRL3) or variable region sequences as described in Aspect 1A hereinbelow.

In another embodiment, the bispecific antibody binds another target antigen which is ICOS and the binding to ICOS is provided by an antigen-binding domain (for example, a V_{H}, a V_{L} or a paired V_{H} and V_{L}) having any of the sequences, including CDR sequences (for example CDRH3 and/or CDRL3) or variable region sequences as described in arrangement 5 and arrangement 5a hereinbelow, and any of the anti-ICOS antibodies described in sentences 1 to 102 and sentences 1a to 21a.

In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds hPD-L1 (optionally wherein the antibody has a structure as defined in any one of concepts 1 to 40, or wherein the antibody has a sequence-including CDRs and variable regions—as defined in Aspect 1a hereinbelow) and a Fab which binds GITR (optionally wherein the GITR Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds GITR (optionally wherein the GITR antibody has a sequence—including CDRs and variable regions—as defined in Aspect 1a hereinbelow) and a Fab which binds hPD-L1 (optionally wherein the antibody has a structure as defined in any one of concepts 1 to 40, or wherein the antibody has a sequence—including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 to 31).

In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds ICOS (e.g. binds with agonistic activity and optionally wherein the ICOS Fab has a sequence—including CDRs and variable regions—as defined in arrangement 5, or in arrangement 5a, or in sentences 1 to 102, or in sentences 1a to 21a hereinbelow). In one embodiment, the ICOS Fab has a sequence of any of the ICOS antibodies described herein in sentences 1 to 102 or in sentences 1a to 21a) In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds ICOS (e.g. binds with agonistic activity or optionally wherein the ICOS antibody has a sequence—including CDRs and variable regions—as defined in arrangement 5, or in arrangement 5a, or in sentences 1 to 102, or in sentences 1a to 21a hereinbelow) and a Fab which binds hPD-L1 (optionally wherein the antibody has a structure as defined in any one of concepts 1 to 40, or wherein the antibody has a sequence—including CDRs and variable regions—as defined in Aspect 1A hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds TIM-3 (optionally wherein the TIM-3 Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds TIM-3 (optionally wherein the TIM-3 antibody has a sequence-including CDRs and variable regions—as defined in Aspect 1a hereinbelow) and a Fab which binds hPD-L1 (optionally wherein the antibody has a structure as defined in any one of concepts 1 to 40, or wherein the antibody has a sequence—including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds CD137 (optionally wherein the CD137 Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds CD137 (optionally wherein the CD137 antibody has a sequence-including CDRs and variable regions—as defined in Aspect 1a hereinbelow) and a Fab which binds hPD-L1 (optionally wherein the antibody has a structure as defined in any one of concepts 1 to 40, or wherein the antibody has a sequence—including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds CD3 (optionally wherein the CD3 Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds CD3 (optionally wherein the CD3 antibody has a sequence—including CDRs and variable regions—as defined in Aspect 1a hereinbelow) and a Fab which binds hPD-L1 (optionally wherein the antibody has a structure as defined in any one of concepts 1 to 40, or wherein the antibody has a sequence—including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

Any of the targets listed above (and the Fabs and/or full antibodies described in more detail in Aspect 1A) may be applied to the FIT-Ig structure.

Concept 40. The bispecific antibody according to concept 39, wherein the another target antigen is TIGIT or LAG3.

In any of concepts 37 to 40, if the antibody or fragment thereof has the heavy and light variable region sequences of 84G09, then the bispecific antibody shall be interpreted as not including a mAb² format wherein the Fcab has binding affinity to LAG3.

In one embodiment, the bispecific antibody has a FIT-Ig format which comprises a full antibody (e.g. an antibody comprising a light chain comprising a V_{L} and C_{L} and a heavy chain comprising V_{H}, C_{H}1, C_{H}2 and C_{H}3) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds TIGIT (optionally wherein the TIGIT Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH1, CH2 and CH3) which binds TIGIT (optionally wherein the TIGIT antibody has a sequence—including CDRs and variable regions—as defined in Aspect 1a hereinbelow) and a Fab which binds hPD-L1 (optionally wherein the antibody has a structure as defined in any one of concepts 1 to 40, or wherein the antibody has a sequence-including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

In one embodiment, the bispecific antibody has a FIT-Ig format which comprises a full antibody (e.g. an antibody comprising a light chain comprising a V_{L} and C_{L} and a heavy chain comprising V_{H}, C_{H}1, C_{H}2 and C_{H}3) which binds hPD-L1 (optionally wherein the antibody has a structure as defined in any one of concepts 1 to 40, or wherein the antibody has a sequence—including CDRs and variable regions—as defined in Aspect 1a hereinbelow) and a Fab which binds LAG3 (optionally wherein the LAG3 Fab has a sequence—including CDRs and variable regions—as defined in Aspect 1a hereinbelow). In one embodiment, the bispecific antibody has a FIT-Ig format which comprises a full antibody (e.g. an antibody comprising a light chain comprising a V_{L} and C_{L} and a heavy chain comprising V_{H}, CH₁, CH₂ and CH₃) which binds LAG3 (optionally wherein the LAG3 antibody has a sequence—including CDRs and variable regions—as defined in Aspect 1a hereinbelow) and a Fab which binds hPD-L1 (optionally wherein the antibody has a structure as defined in any one of concepts 1 to 40, or wherein the antibody has a sequence-including CDRs and variable regions—as defined in Aspect 1a hereinbelow). In one embodiment, the FIT-Ig is effector-enabled (e.g. as described in any of concepts 30 to 32). In another embodiment, the FIT-Ig is effector-disabled (e.g. is an IgG4 format, or as described in any of concepts 30 or 31).

Concept 41. An antibody or fragment as defined in any preceding concept for use in treating or preventing a hPD-L1-mediated disease or condition, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

Concept 42. Use of an antibody or fragment as defined in any one of concepts 1 to 40 in the manufacture of a medicament for administration to a human for treating or preventing a hPD-L1 mediated disease or condition in the human, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

Concept 43. A method of treating or preventing a hPD-L1 mediated disease or condition, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas) in a human, comprising administering to said human a therapeutically effective amount of an antibody or fragment as defined in any one of concepts 1 to 40, wherein the hPD-L1 mediated disease or condition is thereby treated or prevented.

In any of concepts 41 to 43, the hPD-L1 mediated disease may be any of those as described herein. In one embodiment, in any of concepts 41 to 43, the hPD-L1 mediated disease is a virally induced cancer, such as cervical cancer and nasopharyngeal cancer, for example cervical cancers caused by HPV infection. In one embodiment, in any of concepts 41 to 43, the hPD-L1 mediated disease is a chronic viral infection. In one embodiment, in any of concepts 41 to 43, the hPD-L1 mediated disease is a neoplastic disease. In one embodiment, in any of concepts 41 to 43, the hPD-L1 mediated disease is a non-neoplastic disease. In one embodiment, in any of concepts 41 to 43, the hPD-L1 mediated disease is a malignant tumour. In one embodiment, in any of concepts 41 to 43, the hPD-L1 mediated disease is a cancer which is known to be responsive to PD-L1 therapy, such as melanoma, Merkel cell carcinoma, non-small cell lung cancer (squamous and non-squamous), renal cell cancer, bladder cancer, head and neck squamous cell carcinoma, mesothelioma. In one embodiment, in any of concepts 41 to 43, the hPD-L1 mediated disease is a cancer which is a soft tissue sarcoma.

Concept 44. The antibody or fragment according to concept 41, the use according to concept 42 or the method according to concept 43, wherein the hPD-L1-mediated disease or condition is cancer.

Concept 44a. The antibody or fragment according to concept 41, the use according to concept 42 or the method according to concept 43, wherein the hPD-L1-mediated disease or condition is a neurodegenerative disease, disorder or condition, optionally wherein the neurodegenerative disease, disorder or condition is selected from Alzheimer's disease, amyotrophic lateral sclerosis, Parkinson's disease, Huntington's disease, primary progressive multiple sclerosis, secondary progressive multiple sclerosis, corticobasal degeneration, Rett syndrome, a retinal degeneration disorder selected from age-related macular degeneration and retinitis pigmentosa; anterior ischemic optic neuropathy, glaucoma, uveitis, depression, trauma-associated stress or post-traumatic stress disorder, frontotemporal dementia, Lewy body dementias, mild cognitive impairments, posterior cortical atrophy, primary progressive aphasia and progressive supranuclear palsy or aged-related dementia, in particular Alzheimer's disease, amyotrophic lateral sclerosis, Parkinson's disease and Huntington's disease, and e.g. Alzheimer's disease.

In concept 44a, the therapeutically effective amount of an antibody or fragment may comprise an antigen-binding site that specifically binds PD-L1, e.g. hPD-L1.

In one embodiment, the PD-L1 antigen-binding site comprises the CDRH1, CDRH2, CDR3, CDRL1, CDRL2 and CDRL3, or the V_{H}, or the V_{L} or the V_{H} and V_{L} region from any one of the anti-PD-L1 antibodies selected from atezolizumab (Roche), avelumab (Merck), BMS-936559/MDX-1105 (BMS), durvalumab/Medi4736 (Medimmune), KN-035, CA-170, FAZ-053, M7824, ABBV-368, LY-3300054, GNS-1480, YW243.55.S70, REGN3504 and any of the PD-L1 antibodies disclosed in WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 or WO2007/005874, which antibodies and sequences are incorporated herein by reference.

Concept 45. The antibody or fragment, the use or the method according to concept 44, wherein the cancer is selected from melanoma, Merkel cell carcinoma, non-small cell lung cancer (squamous and non-squamous), renal cell cancer, bladder cancer, head and neck squamous cell carcinoma and mesothelioma or is selected from virally induced cancers (such as cervical cancer and nasopharyngeal cancer) and soft tissue sarcomas.

Concept 46. The antibody or fragment, use or the method according to any one of concepts 41 to 45, further comprising administering to the human a further therapy, for example a further therapeutic agent, optionally wherein the further therapeutic agent is independently selected from the group consisting of:
a. other immune checkpoint inhibitors (such as anti-TIM-3 antibodies, anti-CTLA-4 antibodies, anti-TIGIT antibodies and anti-LAG-3 antibodies);
b. immune stimulators (such as anti-OX40 antibodies, anti-GITR antibodies, anti-CD137 antibodies, anti-ICOS antibodies and anti-CD40 antibodies);
c. chemokine receptor antagonists (such as CXCR4, CCR4 and CXCR2);
d. targeted kinase inhibitors (such as CSF-1R or VEGFR inhibitors);
e. angiogenesis inhibitors (such as anti-VEGF-A or Delta-like Ligand-4);
f. immune stimulating peptides or chemokines (such as CXCL9 or CXCL10);
g. cytokines (such as IL-15 and IL-21);
h. bispecific T-cell engagers (BiTEs) having at least one specificity against CD3 (e.g. CD3/CD19 BITE);
i. other bi-specific molecules (for example IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
j. oncolytic viruses (such as HSV virus (optionally which secretes GMCSF), Newcastle disease virus and Vaccinia virus);
k. vaccination with tumour associated antigens (such as New York Esophageal Cancer-1 [NY-ESO-1], Melanoma Associated Antigen-3 [MAGE-3]);
l. cell-based therapies (such as chimeric Antigen Receptor-T cells (CAR-T) for example expressing anti-CD19, anti-EpCam or anti-mesothelin);
m. bispecific NK cell engagers having a specificity against an activating MK receptor such as NKG2D or CD16a; and
n. adoptive transfer of tumour specific T-cells or LAK cells,
   or optionally wherein the further therapy is chemotherapy, radiotherapy and surgical removal of tumours. Radiotherapy may be single dose or in fractionated doses, either delivered to affected tissues directly or to the whole body.

Chemotherapeutic agents may any as described hereinabove, in particular agents that induce immunogenic cell death, for example platinum therapies, such as oxaliplatin. In one embodiment, the chemotherapy is a standard of care cytotoxic chemotherapy for the cancer being treated.

In this aspect, the bispecific molecules include “bispecific antibodies” and antibody fusion proteins, including those formats and molecules described in concepts 37 to 40. The antibodies may be any of the sequences or antibodies described in arrangement 5, 5a or detailed in aspect 1a.

The further therapeutic agents of this concept may be delivered by any method, which methods are well-known to those skilled in the art. For example, the further therapeutic agents may be delivered orally, systemically or locally (to the tumour environment). In one embodiment, the further therapeutic agent is delivered orally. In one embodiment, the further therapeutic agent is delivered systemically (e.g. intravenously). In one embodiment, the further therapeutic agent is delivered locally to the tumour environment.

Compositions and routes of administration are described in more detail hereinbelow.

Concept 47. The antibody or fragment, use or the method according to concept 46, wherein the further therapeutic agent is administered sequentially or simultaneously with the anti-hPD-L1 antibody or fragment.

Concept 48. A pharmaceutical composition comprising an antibody of fragment as defined in any one of concepts 1 to 40 and a pharmaceutically acceptable excipient, diluent or carrier and optionally further comprising a further therapeutic agent independently selected from the group consisting of:
a) other immune checkpoint inhibitors (such as anti-TIM-3 antibodies, anti-CTLA-4 antibodies, anti-TIGIT antibodies and anti-LAG-3 antibodies);
b) immune stimulators (such as anti-OX40 antibodies, anti-GITR antibodies, anti-CD137 antibodies, anti-ICOS antibodies and anti-CD40 antibodies);
c) chemokine receptor antagonists (such as CXCR4, CCR4 and CXCR2);
d) targeted kinase inhibitors (such as CSF-1R or VEGFR inhibitors);
e) angiogenesis inhibitors (such as anti-VEGF-A or Delta-like Ligand-4);
f) immune stimulating peptides or chemokines (such as CXCL9 or CXCL10);
g) cytokines (such as IL-15 and IL-21);
h) bispecific T-cell engagers (BiTEs) having at least one specificity against CD3 (e.g. CD3/CD19 BiTE);
i) other bi-specific molecules (for example IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
j) oncolytic viruses (such as HSV virus (optionally which secretes GMCSF), Newcastle disease virus and Vaccinia virus);
k) vaccination with tumour associated antigens (such as New York Esophageal Cancer-1 [NY-ESO-1], Melanoma Associated Antigen-3 [MAGE-3]);
l) cell-based therapies (such as chimeric Antigen Receptor-T cells (CAR-T) for example expressing anti-CD19, anti-EpCam or anti-mesothelin);
m) bispecific NK cell engagers having a specificity against an activating MK receptor such as NKG2D or CD16a;
and
n) adoptive transfer of tumour specific T-cells or LAK cells.

Pharmaceutical formulations are well-known to those skilled in the art. In one embodiment, the antibody or fragment is administered intravenously. In one embodiment, the antibody or fragment is administered subcutaneously.

In an example, an antibody or fragment as disclosed herein is contained in a medical container, e.g., a vial, syringe, IV container or an injection device (such as an intraocular or intravitreal injection device). In an example, the antibody or fragment is in vitro, for example, in a sterile container.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous. Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In this aspect, the bispecific molecules include “bispecific antibodies” and antibody fusion proteins, including those formats and molecules described in concepts 37 to 40. The further therapeutic agents of this concept may be delivered by any method, which methods are well-known to those skilled in the art. For example, the further therapeutic agents may be delivered orally, systemically or locally (to the tumour environment). In one embodiment, the further therapeutic agent is delivered orally. In one embodiment, the further therapeutic agent is delivered systemically (e.g. intravenously). In one embodiment, the further therapeutic agent is delivered locally to the tumour environment.

The antibodies may have any of the sequences or may be any of the antibodies described in arrangement 5, 5a or detailed in aspect 1a.

Concept 49. A pharmaceutical composition according to concept 48, or a kit comprising a pharmaceutical composition as defined in concept 48, wherein the composition is for treating and/or preventing a hPD-L1-mediated condition or disease, e.g. selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease, diffuse large B-cell lymphoma.

Concept 50. A pharmaceutical composition according to concept 48 or concept 49 in combination with, or kit according to concept 49 comprising, a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody or fragment.

Concept 51. A method of modulating PD-1/PD-L1 interaction in a patient, comprising administering an effective amount of an antibody or fragment as defined in any one of concepts 1 to 40 to said patient.

In another embodiment, there is provided a method of modulating CD80/PD-L1 interaction in a patient, comprising administering an effective amount of an antibody or fragment as defined in any one of concepts 1 to 40 to said patient. In another embodiment, the antibody or fragment modulates CD80/PD-L1 interaction, but does not modulate PD-1/PD-L1 interaction. In another embodiment, the antibody or fragment blocks CD80/PD-L1 interaction, but does not block PD-1/PD-L1 interaction. In another embodiment, the antibody or fragment inhibits CD80/PD-L1 interaction, but does not inhibit PD-1/PD-L1 interaction.

Concept 52. A method of inhibiting PD-L1 activity in a patient, comprising administering an effective amount of an antibody or fragment as defined in any one of concepts 1 to 40 to said patient. In one embodiment, the antibody or fragment blocks or inhibits PD-1 binding to PD-L1. In one embodiment, the antibody or fragment blocks or inhibits CD80 binding to PD-L1.

Concept 53. A method of treating a proliferative disease in an animal (e.g. a human), comprising administering an effective amount of an antibody or fragment as defined in any one of concepts 1 to 40 to said patient. Proliferative diseases may be any as described elsewhere herein.

Concept 54. A method of detecting PD-L1 expression in a sample, comprising contacting the sample with an antibody or fragment as defined in any one of concepts 1 to 40.

Concept 55. A method comprising contacting a biological sample with an antibody or fragment as defined in any one of concepts 1 to 40 to form a complex with PD-L1 present in the sample and measuring the presence, absence or level of the complex in the biological sample.

Concept 56. The method according to concept 55, wherein the presence, absence and/or level of PD-L1 expression is detected prior to treatment and a high level of surface expressed PD-L1 is indicative of successful treatment.

Concept 57. The method according to concept 55, wherein the presence, absence and/or level of PD-L1 expression is detected during treatment as an early response biomarker.

Concept 58. The method according to concept 55 or concept 57, wherein the presence, absence and/or level of PD-L1 expression is detected during or after treatment to help determine one or more of: whether treatment has been successful, whether treatment should continue, and/or whether treatment should be modified.

Concept 59. The method according to any one of concepts 55 to 58, wherein therapy comprises treatment with an anti-PD-L1 antibody, optionally as defined in any one of concepts 1 to 40.

Concept 60. A method for monitoring therapy efficacy, the method comprising detecting expression of surface expressed PD-L1 in a patient prior to therapy, and during or after therapy, wherein an antibody or fragment as defined in any one of concepts 1 to 40 is used to detect expression of surface expressed PD-L1.

Concept 61. The method according to concept 60, wherein surface expressed PD-L1 expression is detected in vivo.

Concept 62. The method according to concept 60, wherein surface expressed PD-L1 expression is detected in a tissue sample in vitro.

Concept 63. A method for identifying binding partners for PD-L1, the method comprising immunoprecipitating an intact protein complex comprising PD-L1 using an antibody or fragment as defined in any one of concepts 1 to 40.

Concept 64. A method of diagnosing a disease in a human subject associated with altered PD-L1 expression comprising the steps of contacting a biological sample from the human subject with an antibody as defined in concepts 1 to 40 to form a complex between the antibody and PD-L1 present in the sample; and detecting the amount of the complex.

Concept 65. A nucleic acid that encodes the CDRH3 of an antibody or fragment as defined in any one of concepts 1 to 40.

Concept 65a. There is also provided a nucleic acid that encodes the CDRH2 of an antibody or fragment as defined in any one of concepts 1 to 40.

Concept 65b. There is also provided a nucleic acid that encodes the CDRH1 of an antibody or fragment as defined in any one of concepts 1 to 40.

Concept 65c. There is also provided a nucleic acid that encodes the CDRL1 of an antibody or fragment as defined in any one of concepts 1 to 40.

Concept 65d. There is also provided a nucleic acid that encodes the CDRL2 of an antibody or fragment as defined in any one of concepts 1 to 40.

Concept 65e. There is also provided a nucleic acid that encodes the CDRL3 of an antibody or fragment as defined in any one of concepts 1 to 40. In one embodiment, the nucleic acid is an isolated and purified nucleic acid.

Concept 66. A nucleic acid that encodes a V_{H} domain and/or a V_{L} domain of an antibody or fragment as defined in any one of concepts 1 to 40. The V_{H} and V_{L} domain nucleic acid sequences of the invention are provided in the sequence listing. In one embodiment, the nucleic acid sequence is at least 70% identical to the specified Seq ID No. In one embodiment, the nucleic acid sequence is at least 75% identical to the specified Seq ID No. In one embodiment, the nucleic acid sequence is at least 95% identical to the specified Seq ID No. In one embodiment, the nucleic acid sequence is at least 96% identical to the specified Seq ID No. In one embodiment, the nucleic acid sequence is at least 97% identical to the specified Seq ID No. In one embodiment, the nucleic acid sequence is at least 98% identical to the specified Seq ID No. In one embodiment, the nucleic acid sequence is at least 99% identical to the specified Seq ID No. In one embodiment, the nucleic acid sequence is at least 99.5% identical to the specified Seq ID No.

Concept 67. The nucleic acid according to concept 66 comprising a nucleotide sequence that is at least 80% identical to the sequence of SEQ ID NO:36 and/or SEQ ID NO: 46.

Concept 67a. A nucleic acid according to concept 66 comprising a nucleotide sequence that is at least 80% identical to the sequence of SEQ ID NO:16 and/or SEQ ID NO: 26.

Concept 67b. A nucleic acid according to concept 66 comprising a nucleotide sequence that is at least 80% identical to the sequence of SEQ ID NO:61 and/or SEQ ID NO: 71.

Concept 67c. A nucleic acid according to concept 66 comprising a nucleotide sequence that is at least 80% identical to the sequence of SEQ ID NO:81 and/or SEQ ID NO: 91.

Concept 67d. A nucleic acid according to concept 66 comprising a nucleotide sequence that is at least 80% identical to the sequence of SEQ ID NO:101 and/or SEQ ID NO: 111.

Concept 67e. A nucleic acid according to concept 66 comprising a nucleotide sequence that is at least 80% identical to the sequence of SEQ ID NO:121 and/or SEQ ID NO: 131.

Concept 67f. A nucleic acid according to concept 66 comprising a nucleotide sequence that is at least 80% identical to the sequence of SEQ ID NO:161 and/or SEQ ID NO: 171.

Concept 67g. A nucleic acid according to concept 66 comprising a nucleotide sequence that is at least 80% identical to the sequence of SEQ ID NO:181 and/or SEQ ID NO: 191.

Concept 67h. A nucleic acid according to concept 66 comprising a nucleotide sequence that is at least 80% identical to the sequence of SEQ ID NO:141 and/or SEQ ID NO: 151.

Concept 67i. A nucleic acid according to concept 66 comprising a nucleotide sequence that is at least 80% identical to the sequence of SEQ ID NO:247 and/or SEQ ID NO: 257.

Concept 67j. A nucleic acid according to concept 66 comprising a nucleotide sequence that is at least 80% identical to the sequence of SEQ ID NO:267 and/or SEQ ID NO: 277.

Concept 67k. A nucleic acid according to concept 66 comprising a nucleotide sequence that is at least 80% identical to the sequence of SEQ ID NO:287 and/or SEQ ID NO: 297.

Concept 67l. A nucleic acid according to concept 66 comprising a nucleotide sequence that is at least 80% identical to the sequence of SEQ ID NO:352 and/or SEQ ID NO: 362.

In one embodiment, the nucleic acid sequence is at least 70% identical to the specified Seq ID No. In one embodiment, the nucleic acid sequence is at least 75% identical to the specified Seq ID No. In one embodiment, the nucleic acid sequence is at least 95% identical to the specified Seq ID No. In one embodiment, the nucleic acid sequence is at least 96% identical to the specified Seq ID No. In one embodiment, the nucleic acid sequence is at least 97% identical to the specified Seq ID No. In one embodiment, the nucleic acid sequence is at least 98% identical to the specified Seq ID No. In one embodiment, the nucleic acid sequence is at least 99% identical to the specified Seq ID No. In one embodiment, the nucleic acid sequence is at least 99.5% identical to the specified Seq ID No.

Concept 68. A nucleic acid that encodes a heavy chain or a light chain of an antibody as defined in any one of concepts 1 to 40.

Concept 69. A vector comprising the nucleic acid of any one of concepts 65 to 68; optionally wherein the vector is a CHO or HEK293 vector.

Concept 70. A host comprising the nucleic acid of any one of concepts 65 to 68 or the vector of concept 69.

### Immunocytokines

In a first configuration, there is provided an immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
wherein the V_{H} domain and V_{L} domain are comprised by an antigen-binding site that specifically binds to hPD-L1 as defined by Seq ID No:1, and competes for binding to said hPD-L1 with the antibody 1D05; and
   wherein the immunocytokine comprises a V_{H} domain which comprises a CDRH3 comprising the motif X₁GSGX₂YGX₃X₄FD, wherein X₁, X₂ and X₃ are independently any amino acid, and X₄ is either present or absent, and if present, may be any amino acid (SEQ ID NO: 563). In a second configuration, there is provided an immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
   wherein the V_{H} domain and V_{L} domain are comprised by an antigen-binding site that specifically binds to hPD-L1, and competes for binding to said hPD-L1 with the antibody 1D05, wherein the antibody or fragment comprises a V_{H} domain which comprises the CDRH3 sequence of SEQ ID NO:29 or 32, or the CDRH3 sequence of SEQ ID NO:29 or 32 comprising 6 or fewer amino acid substitutions.

In a third configuration, there is provided an immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
wherein the V_{H} domain and V_{L} domain are comprised by an antigen-binding site that specifically binds to hPD-L1; and
wherein the V_{H} domain comprises a CDRH3 of from 12 to 20 amino acids and which is derived from the recombination of a human V_{H} gene segment, a human D gene segment and a human J_{H} gene segment, wherein the human J_{H} gene segment is IGHJ5 (e.g. IGHJ5*02).

In a fourth configuration, there is provided an immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
   wherein the V_{H} domain and V_{L} domain are comprised by an antigen-binding site that specifically binds to an epitope that is identical to an epitope to which the antibody 1D05 specifically binds.

In a fifth configuration, there is provided an immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
   wherein the V_{H} domain and V_{L} domain are comprised by an antigen-binding site which competes for binding to hPD-L1 with the antibody 1D05.

In a sixth configuration, there is provided an immunocytokine as defined in any other configuration, embodiment or aspect for use in treating or preventing a hPD-L1-mediated disease or condition.

In a seventh configuration, there is provided the use of an immunocytokine as defined in any other configuration, embodiment or aspect in the manufacture of a medicament for administration to a human for treating or preventing a hPD-L1 mediated disease or condition in the human.

In an eighth configuration, there is provided a method of treating or preventing a hPD-L1 mediated disease or condition in a human, comprising administering to said human a therapeutically effective amount of an immunocytokine as defined in any other configuration, embodiment or aspect, wherein the hPD-L1 mediated disease or condition is thereby treated or prevented.

In a ninth configuration, there is provided a pharmaceutical composition comprising an immunocytokine as defined in any other configuration, embodiment or aspect, and a pharmaceutically acceptable excipient, diluent or carrier.

In a tenth configuration, there is provided a kit comprising a pharmaceutical composition comprising an immunocytokine as defined in any other configuration, embodiment or aspect, and a pharmaceutically acceptable excipient, diluent or carrier. In an eleventh configuration, there is provided a nucleic acid that encodes a heavy chain and/or a light chain of an immunocytokine as defined in any other configuration, embodiment or aspect.

In a twelfth configuration, there is provided a vector comprising the nucleic acid that encodes a heavy chain and/or a light chain of an immunocytokine as defined in any other configuration, embodiment or aspect.

In a thirteenth configuration, there is provided a host comprising the nucleic acid of any other configuration, embodiment or aspect or the vector as defined in any other configuration, embodiment or aspect.

The immunocytokines comprise a cytokine molecule, which may be IL-2 or a variant thereof (including variant having a 1 to 10 amino acid deletion at the N-terminus). The antibodies as described hereinabove may be used in any immunocytokine described herein. Without being bound by theory, immunocytokines of the invention may provide one or more of the following advantageous properties:
synergistic activity (by virtue of the therapeutic activity of antibody Fab portion in combination with the cytokine)
improved tumour targeting
ability to retain effector functions such as CDC, ADCC and/or ADCP
reduced off-target effects
reduced toxicity (e.g. compared to free cytokine or cytokine when fused to the heavy chain of an immunocytokine)
reduced immunogenicity
lower dose/frequency of dosing, in particular due to improved half life of light chain cytokine fusions as compared to heavy chain fusion equivalents
Specificity for blocking only one of the ligands of PD-L1 (e.g. blocks CD80/PD-L1 interaction, but not PD-1/PD-L1 interaction)
Solubility
Stability
Ease of formulation
Frequency of dosing and/or route of administration
Manufacturability (e.g. expression, ease of purification, isoforms)

1D05 ICK comprises a heavy chain amino acid sequence of Seq ID No:299, and a light chain amino acid sequence of Seq ID No:300. The light chain comprises a V_{L} domain comprising the CDRs and V_{L} sequence of antibody 1D05 described hereinabove, fused at the heavy chain to full length, wild-type, human IL-2 cytokine. It does not contain a linker peptide. The heavy chain comprises a V_{H} domain comprising the CDRs and V_{H} sequence of antibody 1D05 described hereinabove, fused to a disabled IgG constant region (Seq ID No: 205).

The IL-2 binding portion of an immunocytokine may be a variant IL-2, in particular an IL-2 having an R38A mutation (as described in amino acids 21-133 of the variant IL-2 described as SEQ ID NO:517) or an R38Q mutation (as described in amino acids 21-133 of the variant IL-2 described as SEQ ID NO:518).

Immunocytokines may be described in the following sentences or aspects. Unless otherwise apparent, the features of any of the concepts described hereinabove apply mutatis mutandis to any of the aspects hereinbelow.

Aspect 1. An immunocytokine comprising an immunoglobulin heavy chain and an immunoglobulin light chain, wherein the heavy chain comprises in N- to C-terminal direction:
A V_{H} domain comprising CDRH1, CDRH2 and CDRH3; and
A heavy chain constant region;
and wherein the light chain comprises in N- to C-terminal direction:
A V_{L} domain comprising CDRL1, CDRL2 and CDRL3;
A light chain constant region, (C_{L});
Optionally, a linker, (L); and
An IL-2 cytokine;
wherein the V_{H} domain and V_{L} domain are comprised by an antigen-binding site that specifically binds to hPD-L1 as defined by Seq ID No:1, and competes for binding to said hPD-L1 with the antibody 1D05; and
wherein the immunocytokine comprises a V_{H} domain which comprises a CDRH3 comprising the motif X₁GSGX₂YGX₃X₄FD, wherein X₁, X₂ and X₃ are independently any amino acid, and X₄ is either present or absent, and if present, may be any amino acid (SEQ ID NO: 563).

In the aspects described herein, CDR sequences may be determined according to any method known to those skilled in the art, such as using the Kabat method, the IMGT method or the Chothia method, each of which are described in more detail herein. In one embodiment, the CDR regions are human CDR regions.

In addition to the CDR regions, the V_{H} and/or V_{L} domains may further comprise framework regions, such as FW1, FW2 and FW3. The V_{H} and/or V_{L} domains may be of any origin described herein, and may be for example, fully human, humanised, murine or camelid. In one embodiment, the V_{H} and/or V_{L} domains are human V_{H} and/or V_{L} domains. CDRs may be of a non-human origin (e.g. mouse origin) and be grafted onto human framework regions. In another embodiment, the CDRs are synthetic.

In another embodiment, V_{H} regions may be selected from the group consisting of an antibody variable domain (e.g., a V_{L} or a V_{H}, an antibody single variable domain (domain antibody or dAb), a camelid V_{HH} antibody single variable domain, a shark immunoglobulin single variable domain (NARV), a Nanobody™ or a camelised V_{H} single variable domain); a T-cell receptor binding domain; an immunoglobulin superfamily domain; an agnathan variable lymphocyte receptor; a fibronectin domain (e.g., an Adnectin™); an antibody constant domain (e.g., a CH3 domain, e.g., a CH2 and/or CH3 of an Fcab™) wherein the constant domain is not a functional CH1 domain; an scFv; an (scFv) 2; an sc-diabody; an scFab; a centyrin and an epitope binding domain derived from a scaffold selected from CTLA-4 (Evibody™); a lipocalin domain; Protein A such as Z-domain of Protein A (e.g., an Affibody™ or SpA); an A-domain (e.g., an Avimer™ or Maxibody™); a heat shock protein (such as and epitope binding domain derived from GroEI and GroES); a transferrin domain (e.g., a trans-body); ankyrin repeat protein (e.g., a DARPin™); peptide aptamer; C-type lectin domain (e.g., Tetranectin™); human γ-crystallin or human ubiquitin (an affilin); a PDZ domain; scorpion toxin; and a kunitz type domain of a human protease inhibitor.

The constant region comprises at least two heavy chain constant region domains selected from CH1, CH2, CH3 and CH4. In one embodiment, the constant region comprises (or consists of) a CH1 domain and a CH2 domain. In one embodiment, the constant region comprises (or consists of) a CH1 domain, a hinge region and a CH2 domain. In one embodiment, the constant region comprises (or consists of) a CH1 domain and a CH3 domain, and optionally a hinge region. In one embodiment, the constant region comprises (or consists of) a CH1 domain and a CH4 domain, and optionally a hinge region. In one embodiment, the constant region comprises (or consists of) a CH1 domain, a CH2 domain and a CH3 domain, and optionally a hinge region. In one embodiment, the constant region comprises (or consists of) a CH1 domain, a CH2 domain and a CH4 domain, and optionally a hinge region. In one embodiment, the constant region comprises (or consists of) a CH1 domain, a CH3 domain and a CH4 domain, and optionally a hinge region. In one embodiment, the constant region comprises (or consists of) a full constant region.

The constant region may be of any isotype described herein, e.g. IgA, IgD, IgE, IgG, and IgM. In one embodiment, the constant region is of any origin described herein, and may be for example, human, murine or camelid. In one embodiment, the constant region is a (full) human constant region. In one embodiment, the constant region is a human IgG constant region. In one embodiment, the constant region is a (full) human IgG1 constant region. In one embodiment, the constant region is an effector null (full) human IgG1 constant region. In one embodiment, the constant region has CDC and/or ADCC and/or ADCP activity. In one embodiment, the constant region is engineered to enhance the CDC and/or ADCC and/or ADCP activity. The constant region may be any of the constant regions described in concepts 30 to 32 hereinabove.

The light chain constant region may be a kappa or lambda light chain constant region. The light chain constant region may be as described in concept 28 hereinabove.

An IL-2 cytokine is a cytokine molecule which confers IL-2 activity on one or both of the intermediate affinity IL-2 Receptor (αβ) and the high affinity IL-2 receptor (αβγ). An IL-2 cytokine includes variant IL-2 cytokines. An IL-2 cytokine may be of human origin or of non-human origin, for example of a non-human mammal, including, but not limit to, primates (e.g. monkeys such a rhesus macaque or cynomolgus), rodents (such as mice, rats and guinea pigs) farm animals, (such as cattle, sheep, pigs, goats, horses, chickens, turkeys, ducks and geese), and domestic mammals (such as dogs and cats). In one embodiment, an IL-2 cytokine is a human IL-2 cytokine.

As used herein, a “variant IL-2 cytokine” is a cytokine having up to 10 amino acids deleted from the N terminal sequence, in combination with up to 5 amino acid substitutions, deletions or additions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 20, or first 15, or first 10 amino acids of the wild-type IL-2 sequence in question), in combination with up to 5 (e.g. 1, 2, 3, 4 or 5) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 15 amino acids of the wild-type IL-2 sequence in question), in combination with up to 5 (e.g. 1, 2, 3, 4 or 5) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 10 amino acids of the wild-type IL-2 sequence in question), in combination with up to 5 (e.g. 1, 2, 3, 4 or 5) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 10 amino acids of the wild-type IL-2 sequence in question), in combination with up to 3 (e.g. 1, 2 or 3) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 10 amino acids of the wild-type IL-2 sequence in question), in combination with up to 2 (e.g. 1 or 2) amino acid substitutions elsewhere in the IL-2 cytokine. In one embodiment, the variant IL-2 cytokine comprises (or consists of) up to 10 (e.g. 1, 2, 3, 4, 5, 6, 7, 8, 9 or 10) amino acid deletions from the N-terminal sequence (e.g. within the first 10 amino acids of the wild-type IL-2 sequence in question), in combination with 1 amino acid substitution elsewhere in the IL-2 cytokine.

In one embodiment, the variant IL-2 cytokine comprises (or consists of) up to 9 (e.g. 1, 2, 3, 4, 5, 6, 7, 8 or 9) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 9 (e.g. 1, 2, 3, 4, 5, 6, 7, 8 or 9) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 9 (e.g. 1, 2, 3, 4, 5, 6, 7, 8 or 9) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 8 (e.g. 1, 2, 3, 4, 5, 6, 7 or 8) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 8 (e.g. 1, 2, 3, 4, 5, 6, 7 or 8) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 8 (e.g. 1, 2, 3, 4, 5, 6, 7 or 8) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 7 (e.g. 1, 2, 3, 4, 5, 6 or 7) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 7 (e.g. 1, 2, 3, 4, 5, 6 or 7) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 7 (e.g. 1, 2, 3, 4, 5, 6 or 7) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 6 (e.g. 1, 2, 3, 4, 5 or 6) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 6 (e.g. 1, 2, 3, 4, 5 or 6) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 6 (e.g. 1, 2, 3, 4, 5 or 6) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 5 (e.g. 1, 2, 3, 4 or 5) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 5 (e.g. 1, 2, 3, 4 or 5) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 5 (e.g. 1, 2, 3, 4 or 5) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 4 (e.g. 1, 2, 3 or 4) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 4 (e.g. 1, 2, 3 or 4) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 4 (e.g. 1, 2, 3 or 4) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) up to 3 (e.g. 1, 2 or 3) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 3 (e.g. 1, 2 or 3) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) up to 3 (e.g. 1, 2 or 3) amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

In one embodiment, the variant IL-2 cytokine comprises (or consists of) 1 or 2 amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) 1 or 2 amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine. In one embodiment, the variant IL-2 cytokine comprises (or consists of) 1 or 2 amino acid deletions from the N-terminal sequence (e.g. within the first 20, or first 15, or first 10 amino acids of the wild-type IL-2 sequence in question), in combination with up to 4 (e.g. 1, 2, 3 or 4) amino acid substitutions elsewhere in the IL-2 cytokine.

Substitutions elsewhere in the IL-2 cytokine are defined further in aspect 44 hereinbelow.

Particular IL-2 cytokines and variant IL-2 cytokines are further defined in aspects 40 to 45 hereinbelow.

The amino acid sequence of the α-chain of human IL-2 is provided in Seq ID No:327. The amino acid sequence of the β-chain of human IL-2 is provided in Seq ID No:328. The amino acid sequence of the γ-chain of human IL-2 is provided in Seq ID No:239.

Aspect 1a. An immunocytokine comprising an immunoglobulin heavy chain and an immunoglobulin light chain, wherein the heavy chain comprises in N- to C-terminal direction:
a) A V_{H} domain comprising CDRH1, CDRH2 and CDRH3; and
b) A heavy chain constant region;
and wherein the light chain comprises in N- to C-terminal direction:
c) A V_{L} domain comprising CDRL1, CDRL2 and CDRL3;
d) A light chain constant region, (C_{L});
e) Optionally, a linker, (L); and
f) An IL-2 cytokine;
wherein the V_{H} domain and V_{L} domain are comprised by an antigen-binding site that specifically binds to an antigen selected from: an immune checkpoint inhibitor (such as PD-1, CTLA-4, TIGIT, TIM-3, LAG-3 and VISTA, e.g. TIGIT, TIM-3 and LAG-3), an immune modulator (such as BTLA, hHVEM, CSF1R, CCR4, CD39, CD40, CD73, CD96, CXCR2, CXCR4, CD200, GARP, SIRPα, CXCL9, CXCL10 and CD155, e.g. GARP, SIRPα, CXCR4, BTLA, hVEM and CSF1R), and an immune activator (such as CD137, GITR, OX40, CD40, CXCR3 (e.g. agonistic activity against CXCR3), CD27, CD3 and ICOS (e.g. agonistic activity against ICOS), for example, ICOS, CD137, GITR and OX40).

Any of the embodiments of aspect 1 apply mutatis mutandis to aspect 1a. Any of the features or embodiments of aspects 2 to 54 apply mutatis mutandis to aspect 1a. Any of the features of the antibodies or other embodiments or features of concepts 1 to 70 apply mutatis mutandis to aspect 1a.

In one embodiment, the antigen-binding site specifically binds PD-L1, e.g. hPD-L1. In one embodiment, the PD-L1 antigen-binding site comprises the CDRH1, CDRH2, CDRH3, CDRL1, CDRL2 and CDRL3, or the V_{H}, or the V_{L} or the V_{H} and V_{L} region from any one of the anti-PD-L1 antibodies selected from atezolizumab/MPDL3280A (Roche), avelumab/MSB0010718C (Merck), BMS-936559/MDX-1105 (BMS), durvalumab/Medi4736 (Medimmune), KN-035, CA-170, FAZ-053 M7824, ABBV-368, LY-3300054, GNS-1480, YW243.55.S70, REGN3504 and any of the PD-L1 antibodies disclosed in WO2017/034916, WO2017/020291, WO2017/020858, WO2017/020801, WO2016/111645, WO2016/050721, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO2015/195163, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 or WO2007/005874, which antibodies and sequences are incorporated herein by reference.

In one embodiment, the antigen-binding site specifically binds ICOS, e.g. hICOS. In one embodiment, the antigen-binding site specifically binds ICOS, e.g. hICOS and is an agonist to ICOS, e.g. hICOS. In one embodiment, the antigen-binding site specifically binds ICOS, e.g. hICOS and is an antagonist to ICOS, e.g. hICOS. In one embodiment, the ICOS antigen-binding site comprises the CDRH1, CDRH2, CDRH3, CDRL1, CDRL2 and CDRL3, or the VH, or the VL or the VH and VL region from any one of the anti-ICOS antibodies described in arrangement 5 and arrangement 5a, and any of the anti-ICOS antibodies described in sentences 1 to 102 and sentences 1a to 21a.

In any of the following embodiments, a particular antigen-binding site specifically binds to a human target. In one embodiment, the antigen-binding site specifically binds an immune checkpoint inhibitor. In one embodiment, the antigen-binding site specifically binds an immune checkpoint inhibitor selected from PD-1, CTLA-4, TIGIT, TIM-3, LAG-3 and VISTA. In one embodiment, the antigen-binding site specifically binds an immune checkpoint inhibitor selected from TIGIT, CTLA-4, TIM-3 and LAG-3.

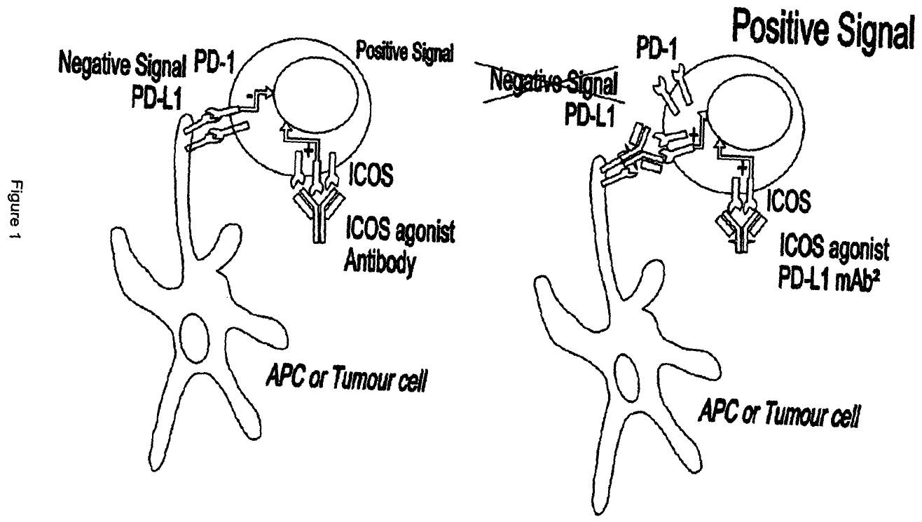

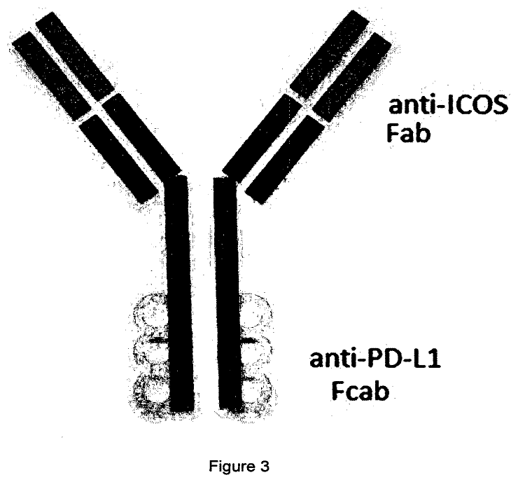

In one embodiment, the antigen-binding site specifically binds PD-1, e.g. human PD-1. In one embodiment, the PD-1 antigen-binding site comprises the CDRH1, CDRH2, CDRH3, CDRL1, CDRL2 and CDRL3, or the VH, or the VL or the VH and VL region from pembrolizumab (Keytruda®/MK-3475), nivolumab (Opdivo®/BMS-936558/MDX-1106), MEDI-0680/AMP514, PDR001, Lambrolizumab, BMS-936558, REGN2810, BGB-A317, BGB-108, PDR-001, SHR-1210, JS-001, JNJ-63723283, AGEN-2034, PF-06801591, genolimzumab, MGA-012, IBI-308, BCD-100, TSR-042 ANA011, AUNP-12, KD033, MCLA-134, mDX400, muDX400, STI-A1110, AB011, 244C8, 388D4, XCE853, or pidilizumab/CT-011, or from any one of the anti-PD-1 antibodies described in WO2015/112800 & US2015/0203579 (including the antibodies in Tables 1 to 3), U.S. Pat. Nos. 9,394,365, 5,897,862 and 7,488,802, WO2017/087599 (including antibody SSI-361 and SHB-617), WO2017/079112, WO2017/071625 (including deposit C2015132, hybridoma LT004, and antibodies 6F5/6 F5 (Re), 6F5H1 L1 and 6F5 H2L2), WO2017/058859 (including PD1AB-1 to PD1AB-6), WO2017/058115 (including 67D9, c67D9, and hu67D9), WO2017/055547 (including 12819.15384, 12748.15381, 12748.16124, 12865.15377, 12892.15378, 12796.15376, 12777.15382, 12760.15375 and 13112.15380), WO2017/040790 (including AGEN2033w, AGEN2034w, AGEN2046w, AGEN2047w, AGEN2001w and AGEN2002w), WO2017/025051 & WO2017/024515 (including 1.7.3 hAb, 1.49.9 hAb, 1.103.11 hAb, 1.103.11-v2 hAb, 1.139.15 hAb and 1.153.7 hAb), WO2017/025016 & WO2017/024465 (including antibody A to antibody I), WO2017/020858 & WO2017/020291 (including 1.4.1, 1.14.4, 1.20.15 and 1.46.11), WO2017/019896 & WO2015/112900 & US2015/0210769 (including BAP049-hum01 to BAP049-hum16 and BAP049-Clone-A to BAP049-Clone-E), WO2017/019846 (including PD-1 mAb 1 to PD-1 mAb 15), WO2017/016497 (including MHC723, MHC724, MHC725, MHC728, MHC729, m136-M13, m136-M19, m245-M3, m245-M5 and m136-M14), WO2016/201051 (including antibody EH12.2H7, antibody hPD-1 mAb2, antibody hPD-1 mAb7, antibody hPD-1 mAb9, antibody hPD-1 mAb15, or an anti-PD-1 antibody selected from Table 1), WO2016/197497 (including DFPD1-1 to DFPD1-13), WO2016/197367 (including 2.74.15 and 2.74.15.hAb4 to 2.74.15.hAb8), WO2016/196173 (including the antibodies in Table 5, and FIGS. 1-5), WO2016/127179 (including R3A1, R3A2, R4B3, and R3D6), WO2016/077397 (including the antibodies described in Table 1 of Example 9), WO2016/106159 (including the murine antibodies in Table 3 of Example 2 and the humanised antibodies in Tables 7, 8 and 9 of Example 3), WO2016/092419 (including C1, C2, C3, EH12.1, mAb7-G4, mAb15-G4, mAb-AAA, mAb15-AAA), WO2016/068801 (including clone A3 and its variants and the other antibodies described in FIGS. 1 to 4), WO2016/014688 (including 10D1, 4C10, 7D3, 13F1, 15H5, 14A6, 22A5, 6E1, 5A8, 7A4, and 7A4D and the humanised antibodies of Examples 9/10), WO2016/015685 (including 10F8, BA08-1, BA-08-2 and 15H6), WO2015/091911 & WO2015/091910 (including the anti-canine PD-1 antibodies in Examples 2, 3 and 4), WO2015/091914 (including the anti-canine PD-1 antibodies in Table 3), WO2015/085847 (including mAb005, H005-1 to H005-4), WO2015/058573 (including cAB7), WO2015/036394 (including LOPD180), WO2015/035606 (including the antibodies in Table 1 of Example 2, in Tables 14, 15 and 16 of Example 7 and in tables 20, 21 and 22 of Example 11), WO2014/194302 (including GA2, RG1B3, RG1H10, RG2A7, RG2H10, SH-A4, RG4A6, GA1, GB1, GB6, GH1, A2, C7, H7, SH-A4, SH-A9, RG1H11, and RG6B), WO2014/179664 (including 9A2, 10B11, 6E9, APE1922, APE1923, APE1924, APE1950, APE1963 and APE2058), WO2014/206107 (including clone 1, 10, 11, 55, 64, 38, 39, 41 and 48), WO2012/135408 (including h409A11, h409A16, and h409A17), WO2012/145493 (including antibodies 1E3, 1E8, 1H3 and h1H3 Var 1 to h1H3 Var 14), WO2011/110621 (including antibody 949 and the modified versions disclosed in FIGS. 1 to 11), WO2011/110604 (including antibody 948 and the modified versions disclosed in FIGS. 3 to 11), WO2010/089411 (including CNCM deposit number 1-4122, 1-4080 or 1-4081), WO2010/036959 (including the antibodies in Table 1 of Example 1), WO2010/029435 & WO2010/029434 (including clones 2, 10 and 19), WO2008/156712 (including hPD-1.08A, hPD-1.09A, h409A11, h409A16 and h409A17 and the antibodies described in Example 2, Table H, Example 4 and table IV), WO2006/121168 (including clones 17D8, 4H1, 5C4, 4A11, 7D3, 5F4, and 2D3), WO2004/004771 or WO2004/056875 (including PD1-17, PD1-28, PD1-33, PD1-35, PD1-F2 and the Abs described in Table 1); the sequences and features of the anti-PD-1 antibodies are incorporated herein by reference.

In one embodiment, the antigen-binding site specifically binds TIGIT, e.g. human TIGIT. In one embodiment, the TIGIT antigen-binding site comprises the CDRH1, CDRH2, CDR3, CDRL1, CDRL2 and CDRL3, or the VH, or the VL or the VH and VL region from RG-6058 (MTIG-7192A) or from any one of the anti-TIGIT antibodies described in WO2017/053748 (including 1A4, 1D3, 4A3, 10A7, 4.1D3.Q1E, h10A7.K4G3, 4.1D3 and the other antibodies described in Examples 1 and 2), WO2017/037707 (including VSIG9 #1 and 258-csl #4), WO2017/030823 (including 14D7, 26B10 and humanized versions in Example 3), WO2016/191643 (including 313R11, 313R12, 313R14, 313R19, 313R20, ATCC PTA-122180 and ATCC PTA-122181), WO2016/106302 (including 14B2, 13E6, 6F9, 11G11, 10C9, 16F6, 11C9, 27A9, 10D7, 20G6, 24E8, 24G1, 27F1, 15A6, 4E4, 13D1, 9B11, 10B8, 22G2, 19H2, 8C8, 17G4, 25E7, 26D8 and 16A8), WO2016/028656 (including 14A6, 28H5 or 31C6 and humanized versions from Example 6), and WO2009/126688 (US2013/0251720, including 10A7 and 1F4); the sequences and features of the anti-TIGIT antibodies are incorporated herein by reference.

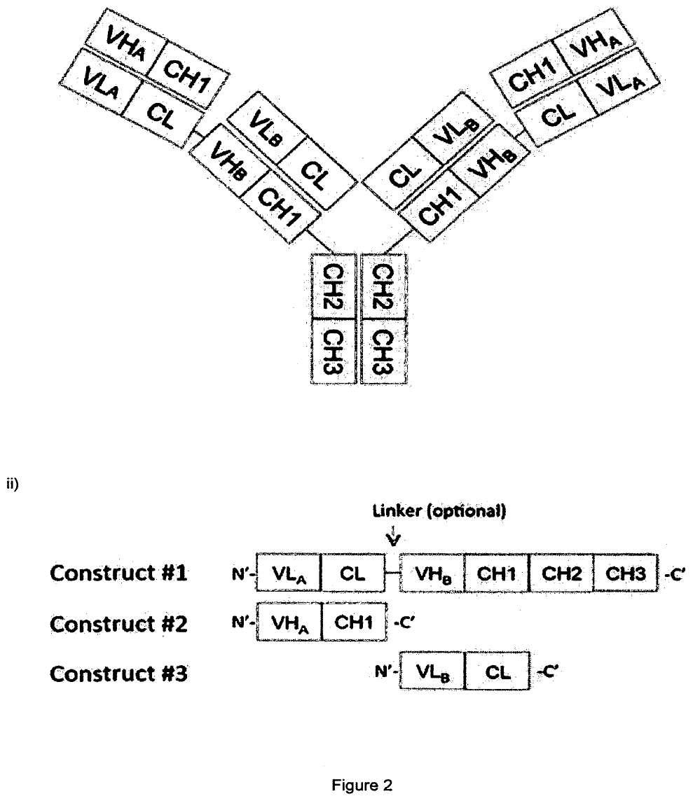

In one embodiment, the antigen-binding site specifically binds TIM-3, e.g. human TIM-3. In one embodiment, the TIM-3 antigen-binding site comprises the CDRH1, CDRH2, CDR3, CDRL1, CDRL2 and CDRL3, or the VH, or the VL or the VH and VL region from F38-2E2 (BioLegend), clone 2E2 (Merck Millipore), clone 6B6E2, clone 024 (Sino Biological) clone 344801 (R&D Systems), clone E-18, clone H-191 (Santa Cruz Biotechnology), or clone 13A224 (United States Biological), TSR-022 (Tesaro) or from any one of the anti-TIM-3 antibodies described in WO2017/079115 (including anti-TIM3 antibodies listed in tables 30-38), WO2017/055404 (including PD1TIM3-0389, PD1TIM3-0168, PD1TIM3-0166, TIM3-0038, TIM3-0018, TIM3-0028, TIM3-0438-Table C), WO2017/031242 (Table 10), WO2016/179194 (including antibodies in FIG. 1b, including mAb F38-2E2 and 2E2), WO2016/171722 (including 344823 and antibodies from the hybridomas 7D11, 10G12, 11G8, 8B.2C12 and 25F.1D6), WO2016/161270 (including APE5137 and APE5121), WO2016/111947 (including mAb5, mAb13, mAb15, mAb17, mAb21, mAb22, mAb26, mAb27, mAb48, mAb58 and mAb91), WO2016/071448 (including TIM3-0016, TIM3-0018, TIM3-0021, TIM3-0022, TIM3-0026, TIM3-0028, TIM3-0030, TIM3-0033, TIM3-0038, TIM3-0433, TIM3-0434, TIM3-0438 and TIM3-0443), WO2016/068802 (including 1B9, 1H9, 1H10, 2C7, 2F4, 2G6, 1D9, 1F4 and 2C8—FIGS. 1, 2 & 3), WO2016/068803 (including A3, B10, G6, G7, G9, A11 and A11_gl—FIGS. 1, 2 & 3), WO2015/117002 (including ABTIM3, ABTIM3-hum02, ABTIM3-hum05, ABTIM3-hum06, ABTIM3-hum09, ABTIM3-hum10, ABTIM3-hum12, ABTIM-hum01, ABTIM-hum04, ABTIM3-hum07, ABTIM3-hum08, ABTIM3-hum04, ABTIM3-hum21, ABTIM3-hum03, ABTIM3-hum11 and antibodies listed in Table 9), WO2015/048312 (including 5D12), WO2014/022332 (including 2C12), WO2013/006490 (including antibodies in Table 1), WO2011/155607 (including 512, 644, 4545, 4177, 8213, 344823 and 34823), WO2003/063792 (including antibody 8B.2C12 and 25F.1D6), WO2017/019897 (including antibody molecules disclosed in Tables 1-4, including ABTIM3, ABTIM3-hum20, ABTIM3-hum22 and ABTIM3-hum23), WO2016/079050 & WO2016/079050 (including Tim3_0022, Tim3_0016, Tim3_0018, Tim3_00122, Tim3_0022, Tim3_0021, Tim3_0028, Tim3_0026, Tim3_0033, Tim3_0038, Tim3_0030, 1.7.E10, F38-2EL and 27-12E12); the sequences and features of the anti-TIM-3 antibodies are incorporated herein by reference.

In one embodiment, the antigen-binding site specifically binds LAG-3, e.g. human LAG-3. In one embodiment, the LAG-3 antigen-binding site comprises the CDRH1, CDRH2, CDR3, CDRL1, CDRL2 and CDRL3, or the V_{H}, or the V_{L} or the V_{H} and V_{L} region from antibody clone 17B4 (Enzo Life Sciences), or clone 333210 (R&D Systems), or clone 14L676 (United States Biological), or C9B7W (PharMingen), or 11E, or IMO321, or mAb C9B7W (BioXcell) or from any one of the anti-LAG-3 antibodies described in WO95/30750, WO2004/078928, WO2008/132601 (including IMP731 Lag-3 Ab, IMP321, A9H12 Lag-3 mAb and 31G11), WO2010/019570 (including 25F7, 26H10, 25E3, 8B7, 11F2 and 17E5), WO2014/140180 (including H5L7, H5L7BW, IMP731 and antibodies in Tables 3 & Table 7), WO2014/179664 (including APE03109), WO2014/008218 (including Lag3.1, Lag3.5, Lag3.6, Lag3.7 and Lag3.8), WO2015/042246, WO2015/116539 (including BMS-986016), WO2015/138920 (including BAP050-hum01 to BAP050-hum20, huBAP050 (Ser), BAP050-hum01-Ser to BAP050-hum20-Ser, BAP050-Clone-F, BAP050-Clone-G, BAP050-Clone-H, BAP050-Clone-I, BAP050-Clone-J, BAP050 and BAP050-chi), WO2015/198312, WO2016/028672 (including Ab1, Ab2, Ab3, Ab4, Ab5, Ab6, Ab7, Ab8 and Ab9), WO2016/126858, WO2016/200782 (including LAG-3 mAb1 to LAG-3 mAb6), WO2017/015560 (including L32D10, L3E3, L3C5, L35D4, L35G6, L33H11, L32A9, L32A4, L3A1 and the antibodies listed in Table 3), WO2017/062888 (including mAb1, H4H15477P, H4H15483P, H4H15484P, H4H15491, H4H17823P, H4H17826P2, H4H17828P2, H4sH15460P, H4sH15462P, H4sH15463P, H4sH15464P, H4sH15466P, H4sH15467P, H4sH15470P, H4sH15475P, H4sH15479P, H4sH15480P, H4sH15482P, H4sH15488P, H4sH15496P2, H4sH15498P2, H4sH15505P2, H4sH15518P2, H4sH15523P2, H4sH15530P2, H4sH15555P2, H4sH15558P2, H4sH15567P2 and H4H17819P), WO2017/019894, WO2017/037203 (including 8E2, 13E2, 34F4, 17B4 and IMP761), WO2017/087589 (including 11B09) or WO2017/087901; the sequences and features of the anti-LAG-3 antibodies are incorporated herein by reference.

In one embodiment, the antigen-binding site specifically binds VISTA, e.g. human VISTA. In one embodiment, the VISTA antigen-binding site comprises the CDRH1, CDRH2, CDR3, CDRL1, CDRL2 and CDRL3, or the V_{H}, or the V_{L} or the V_{H} and V_{L} region from any one of the anti-VISTA antibodies described in WO2016/207717 & WO2015/097536 (including VSTB50, VSTB53, VSTB60, VSTB95, VSTB112, VSTB116, VSTB174, VSTB175, VSTB149, VSTB140 and the antibodies in Table 1A and Examples 7 and 8) and WO2014/190356 (including clone 2D3 and 18C3); the sequences and features of the anti-VISTA antibodies are incorporated herein by reference.

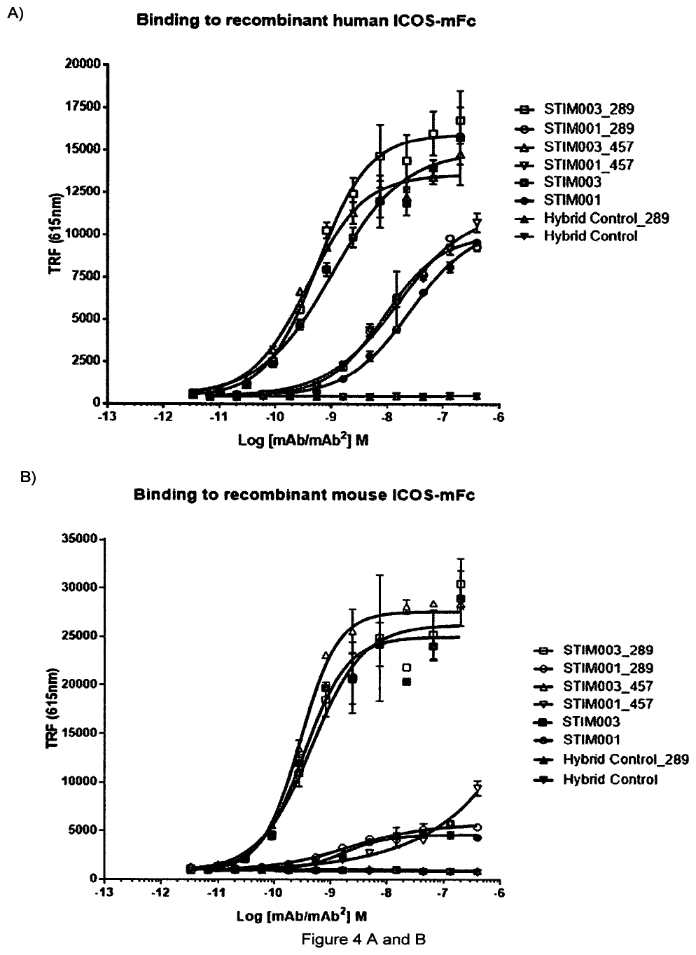

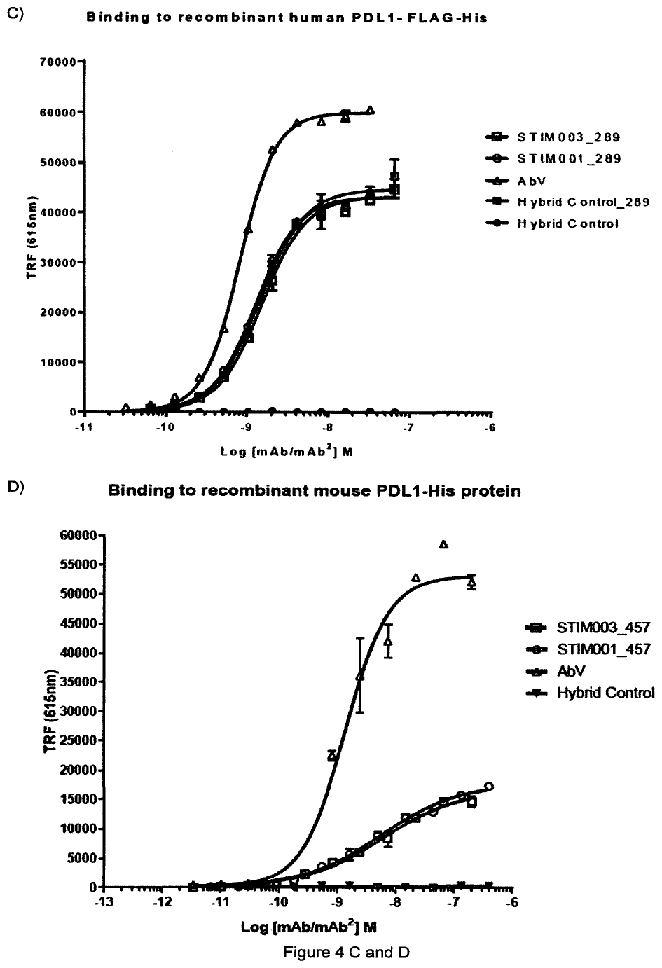

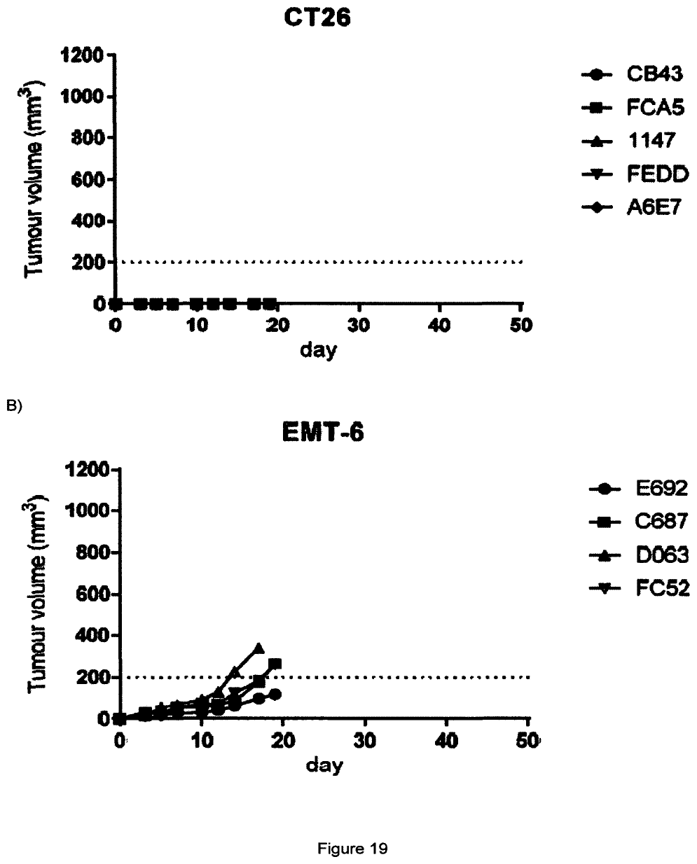

In one embodiment, the antigen-binding site specifically binds CTLA-4, e.g. hCTLA-4. In one embodiment, the CTLA-4 antigen-binding site comprises the CDRH1, CDRH2, CDR3, CDRL1, CDRL2 and CDRL3, or the V_{H}, or the V_{L} or the V_{H} and V_{L} region from ipilimumab (MDX-010, CAS No. 477202-00-9), tremelimumab (ticilimumab/CP-675,206), antibody clone 2F1, clone 1F4 (Abnova Corporation), clone 9H10 (EMD Millipore), clone BNU3 (GeneTex), clone 1 E2, clone AS32 (Lifespan Biosciences) clone A3.4H2.H12 (Acris Antibodies), clone 060 (Sino Biological), clone BU5G3 (Creative Diagnostics), clone MIH8 (MBL International), clone A3.6B10.G1, or clone L3D10 (BioLegend) or from any one of the anti-CTLA-4 antibodies described in WO2017/087588 (ISVs disclosed in FIG. 2), WO2017/084078 (clones C2, C4, C10, C11, C12 and C13, and FIGS. 4-7), WO2016/196237 (including AGEN1884w, AGEN2041w, the sequences in FIGS. 19A, 19B and Tables 1-6), WO2016/130986 & WO2016/130898 (including E8, F7 and the Abs described in Table 4), WO2016/015675 (including hybridoma LT001 and antibodies 8D2, 8D2H1L1, 8D2H2L2, 8D2H3L3, 8D2H2L15 and 8D2H2L17), WO2012/120125 (including 3B10, 8H5, and the Abs identified in Examples 1, 2, 3 and 5), WO2010/097597 (including JMW-3B3 and the variants and fragments disclosed), WO2009/100140 (including 10D1, 1H5, 3A4, 6C10 and the antibodies described in FIGS. 1 to 6), WO2007/008463 & WO2006/101692 & WO2006/101691 & WO2006/048749 & WO2005/09238, (including 4.1.1, 4.8.1, 4.10.2, 4.13.1, 4.14.3, 6.1.1, 11.2.1, 11.6.1, 11.7.1, 12.3.1.1, 12.9.1.1, and 10D1), WO2006/096491 (including ATCC Deposit No. 11.2.1 11.2.1.4 PTA-5169 and 4.1.1 4.1.1.1 PTA-5166), WO2006/066568 (including TGN2122.C, TGN2422.C, 4.8H10H5 and 4.3F6B5 and the antibodies described in tables 3 to 14), WO2006/029219 (including L3D10, L1B11, K4G4, KM10, and YL2), WO2004/029069 (including ATCC deposit number PTA-4537), WO01/54732 (including antibodies 25, 26, 27, 29, 33, 34, 35, 36 and 38), WO01/14424 (including 3A4, 9A5, 2E2, 2E7, 4B6, 4E10, 5C4, 5G1, 11E8, and 11G1 and the antibodies identified in Examples 3 and 4 and table 3) and WO00/37504 (including 3.1.1, 4.1.1, 4.8.1, 4.10.2, 4.13.1, 4.14.3, 6.1.1, 11.2.1, 11.6.1, 11.7.1, 12.3.1.1, and 12.9.1.1); the sequences and features of the anti-CTLA-4 antibodies are incorporated herein by reference.

In one embodiment, the antigen-binding site specifically binds an immune modulator. In one embodiment, the antigen-binding site specifically binds an immune modulator selected from BTLA, hHVEM, CSF1R, CCR4, CD39, CD40, CD73, CD96, CXCR2, CXCR4, CD200, GARP, SIRPα, CXCL9, CXCL10, CXCL11 and CD155, or from BTLA, hHVEM, CSF1R, CCR4, CD39, CD40, CD73, CD96, CXCR2, CXCR4, CD200, GARP, SIRPα, CXCL9, CXCL10 and CD155. In one embodiment, the antigen-binding site specifically binds an immune modulator selected from GARP, SIRPα, CXCR4, BTLA, hVEM and CSF1R.

In one embodiment, the antigen-binding site specifically binds GARP, e.g. human GARP. In one embodiment, the GARP antigen-binding site comprises the CDRH1, CDRH2, CDR3, CDRL1, CDRL2 and CDRL3, or the V_{H}, or the V_{L} or the V_{H} and V_{L} region from G14D9, Plato-1, 272, G6, 50 G10 or 7B11 or from any one of the anti-GARP antibodies described in WO2007/113301 & WO2015/015003 (including MHGARP8, LHG-10, LHG-10-D, LHG-10.3-D, LHG-10.4-D, LHG-10.5-D, LHG-10.6-D, LHG-10.3, LHG-10.4, LHG-10.5, LHG-10.6, 27E10, MHGARP1, MHGARP2, MHGARP3, MHGARP4, MHGARP5, MHGARP6, MHGARP7 and MHGARP9), WO2017/051888 (including 110F, 105F, c151D, c198D, h198D, h151D, h151D-H1L1 and h198D-H3L4); the sequences and features of the anti-GARP antibodies are incorporated herein by reference.

In one embodiment, the antigen-binding site specifically binds SIRPα, e.g. human SIRP□. In one embodiment, the SIRPα antigen-binding site comprises the CDRH1, CDRH2, CDR3, CDRL1, CDRL2 and CDRL3, or the V_{H}, or the V_{L} or the V_{H} and V_{L} region from ED9 (ThermoFisher), or 602411 (Novus Biologicals), or from any one of the anti-SIRPα antibodies described in WO97/48723, WO00/24869 (including 10C4), WO00/66159 (including ED9 and ED17), WO01/40307, WO02/092784 (including SE5A5, SE7C2 and SE12C3), WO2004/108923 (including SE12C3 and 2F34), WO2009/046541 (including P84), WO2011/076781, WO2012/172521, WO2012/040207 (including SE5A5 and mouse P84), WO2013/056352 (including 29-AM4-5, Ab AM4-5, AM5-1, AM5-3, AM5-5, AM5-6, SIRPalpha-AM3-35, AM4-1, SIRP29-AM3-35, SIRP29-AM4-5, SIRP29-AM4-1, 29-AM2-2, 29-AM4-4, 29-AM4-1, 29-AM4-5, 29-AM3-35 and SIRP29-AM3-63), WO2016/063233, WO2016/205042 (including P362) or WO2015/138600 (including KWAR23); the sequences and features of the anti-SIRPα antibodies are incorporated herein by reference.

In one embodiment, the antigen-binding site specifically binds CXCR4, e.g. human CXCR4. In one embodiment, the CXCR4 antigen-binding site comprises the CDRH1, CDRH2, CDR3, CDRL1, CDRL2 and CDRL3, or the V_{H}, or the V_{L} or the V_{H} and V_{L} region of ulocuplumab/BMS-936564, clone 44717.111 or PF-06747143 or from any one of the anti-CXCR4 antibodies described in WO97/49424 (including MAB12G5), WO99/50461, WO01/42308, WO03/066830 & WO2003/066830 (including Ab124 and Ab125), WO2004/059285 (including ALX40-4C), WO2006/089141 (including mAbs 2N, 6R, 18, 19, 20, 33 and 48), WO2007/005605, WO2008/142303 (including MAB170, MAB171, MAB173 and MAB172), WO2008/060367 & WO2013/071068 & WO2015/015401 (including BMS-936564/MDX-1338), WO2009/140124 (including antibody I, II, III, IV and V), WO2009/117706 (including 701, 708, 716, 717, 718 and 4G10), WO2011/161266 (including 4CXCR100, 4CXCR103, 4CXCR104, 4CXCR101, 4CXCR238D2 and 4CXCR238D4), WO2011/098762 (including C-9P21 (Table 1), B-1M22 (Table 2), C1124 (Table 3), D-1K21 (Table 4) and 9N10 (Table 5)), WO2012/175576, WO2013/013025 (including 2A4, 6C7, 4C1, 7C8, 5C9 and 5E1), WO2013/017566 (including Mab 427aB1 and 515H7), WO2013/017562 (including 1-3859 Mab and 515H7), WO2015/069874 (including antibodies corresponding to Seq ID numbers 25 and 29), WO2015/015401 (including 12A11, 6B6, 3G10, m3G10.hIgG1, m3G10.hIgG4, h3G10.A57.hIgG1, h3G10.A57.A58A.hIgG1, h3G10.1.91.A58A.hIgG1, h3G10.1.91.A58B.hIgG1 and h3G10.2.37.2.72.hIgG1), WO2016/156570 (including 281F12, 281A6 and 281D4), WO2016/109872 (including antibodies listed in tables 1, 2, 9 & 12, M3-114-6H, AM4-272-6H, AM3-523-6H, AM4-272, AM3-114, AM3-523, AM4-746 and AM4-1121), WO2017/071625, WO2012/175576, WO2010/125162 & WO2012/055980 & WO2011/121040 & WO2010/037831 (including c414H5 (414H5), c515H7 (515H7) and 301aE5), WO2009/138519 (including ALX40-4C, 238D2, 238D4, 237B5 antibodies and sequences listed in table 1, table 1.1, table A-I, table B-1.1 & B-5), WO2011/042398 (including 238D2 and 238D4), WO2011/083140 (including those disclosed in Tables C-2, C-3, C-4 & C-5, FIG. 2 and ALX-0651, 15H3, 10E12, 10G10, 238B6, 10E9, 281E10, 10A10, 14A2 and 15A1) or WO2011/083141); the sequences and features of the anti-CXCR4 antibodies are incorporated herein by reference.

In one embodiment, the antigen-binding site specifically binds BTLA, e.g. hBTLA. In one embodiment, the BTLA antigen-binding site comprises the CDRH1, CDRH2, CDR3, CDRL1, CDRL2 and CDRL3, or the V_{H}, or the V_{L} or the V_{H} and V_{L} region from antibody clone 1B7, clone 2G8, clone 4C5 (Abnova Corporation), clone 4B8 (antibodies-online), clone MIH26 (Thermo Scientific Pierce Antibodies), clone UMAB61 (OriGene Technologies), clone 330104 (R&D Systems), clone 1B4 (Lifespan Biosciences), clone 440205, clone 5E7 (Creative Diagnostics) or from any one of the anti-BTLA antibodies described in WO2016/176583 (including clone 6F4), WO2011/014438 (including 8D5, 8A3, 20H4, 21H6, 15C5, 19A7 and 4C7), WO2010/106051 (including CNCM deposit number 1-4123) and WO2008/076560 (including 1B4, E4H9, 3C2, 3C2a, 6A5, 11E2, E8D9, 10H6 and 4C9 as detailed in Example 2); the sequences and features of the anti-BTLA antibodies are incorporated herein by reference.

In one embodiment, the antigen-binding site specifically binds hVEM, e.g. human hVEM. In one embodiment, the HVEM antigen-binding site comprises the CDRH1, CDRH2, CDRH3, CDRL1, CDRL2 and CDRL3, or the V_{H}, or the V_{L} or the V_{H} and V_{L} region from any one of the anti-HVEM antibodies described in WO2008/083169 (including LBH1); the sequences and features of the anti-BTLA antibodies are incorporated herein by reference.

In one embodiment, the antigen-binding site specifically binds CSF1R. In one embodiment, the CSF1R antigen-binding site comprises the CDRH1, CDRH2, CDRH3, CDRL1, CDRL2 and CDRL3, or the V_{H}, or the V_{L} or the V_{H} and V_{L} region from any one of the anti-CSF1R antibodies described in WO2009/026303 (including 1.2, 1.109, 2.360 and 1.2.SM and the antibodies in FIGS. 1 and 2), WO2009/112245 (including CXIIG6), WO2011/070024 (including Mab 2F11, 2E10, 2H7 and 1G10, and their derivatives), WO2011/107553 (including 7H5.2G10/DSM ACC2922), WO2011/123381 (including antibody 1 and antibody 2), WO2011/131407 (including 7G5.3B6/DSM ACC2921), WO2011/140249 (including 0301, 0302, and 0311 their derivatives and the antibodies in tables 2, 3 and 5), WO2013/169264 & WO2014/036357 & WO2016/106180 & WO2016/168149 (including huAb1 to huAb16), WO2012/110360 & WO2013/057281 (including CXIIG6, H19K12, H27K5 and H27K15 and the humanised antibodies of tables 1 and 2), WO2013/087699 (including 9D11.2E8 and 10H2.2F12), WO2014/072441 (including H27K15), WO2014/173814 & WO2013/132044 (including Mab 2F11, Mab 2E10, Mab 2H7, Mab 1G10 and sc2-4A5 and the antibodies in Table 3 and 3b), WO2015/028455 & WO2015/028454 (including Ab535, Ab969, and derivatives, e.g. Ab969.g2), WO2015/036511 & WO2016/207312 (including 2F11, 2E10 and the derivatives described in embodiment 33) and WO2017/049038 (including ALM-423 and the antibodies listed in Table 2); the sequences and features of the anti-CSF1R antibodies are incorporated herein by reference.

In one embodiment, the antigen-binding site specifically binds CD39. In one embodiment, the CD39 antigen-binding site comprises the CDRH1, CDRH2, CDR3, CDRL1, CDRL2 and CDRL3, or the V_{H}, or the V_{L} or the V_{H} and V_{L} region from BY40, BY12, BA54g (Biolegend), BU61 (Santa Cruz Biotech), A1 (Ebiosciences), AC2 (Immunotech), 22A9 (Abcam), 24DMS1 or any one of the anti-CD39 antibodies described in WO96/32471, WO00/04041, WO01/10205 (including CD39L4), WO2009/09547 (including CNCM-I-3889/BY40), WO2014/169255, WO2012/085132 (including antibodies VY12, BY40 and BA54g), WO2016/073845 (including R29-5-13A, R29-5-71A, R29-5-165C and R29-9-8B), WO2017/089334 (including 1-391, 1-392 and antibodies produced from hybridomas I-3889 and CNCM I-41171) and WO2009/095478; the sequences and features of the anti-CD39 antibodies are incorporated herein by reference.

In one embodiment, the antigen-binding site specifically binds CD40, e.g. human CD40. In one embodiment, the CD40 antigen-binding site comprises the CDRH1, CDRH2, CDR3, CDRL1, CDRL2 and CDRL3, or the V_{H}, or the V_{L} or the V_{H} and V_{L} region from BMS3h-56-269, CP-870,893, dacetuzumab, SEA-CD40, ADC-1013, RO7009789 and Chi Lob 7/4, or from any one of the anti-CD40 antibodies described in WO2017/059243, WO2017/059196, WO2017/040932, WO2017/040566, WO2017/004016, WO2017/004006, WO2016/196314, WO2016/028810, WO2016/023960, WO2016/023875, WO2015/134988, WO2015/091853, WO2014/070934, WO2014/065403, WO2014/065402, WO2014/04298, WO2013/164789, WO2013/034904, WO2012/149356, WO2012/145673, WO2012/125569, WO2012/111762, WO2012/075111, WO2012/065950, WO2012/041635, WO2011/123489, WO2010/123012, WO2010/104761, WO2010/121231, WO2009/062125, WO2010/104747, WO2010/104748, WO2010/104749, WO2010/024676, WO2009/094391, WO2009/062054, WO2008/091954, WO2007/130493, WO2007/129895, WO2007/124299, WO2007/053767, WO2007/053661, WO2006/128103, WO2006/073443, WO2005/063981, WO2005/063289 (US2012/0263732), WO2005/044855, WO2005/044306, WO2005/044294, WO2005/044307, WO2005/044304, WO2005/044854, WO2005/044305, WO03/040170 (U.S. Pat. Nos. 7,563,442B, 7,618,633B, 7,338,660B, 7,288,251B, 7,626,012B, 8,388,971B, US2013/0024956), WO03/029296, WO02/088186, WO01/83755, WO02/28905, WO02/28480, WO02/28481, WO02/28904, WO01/37870, WO01/16180, WO00/75348 and WO99/42075, WO97/31025, WO95/17202 and WO95/09653; the sequences and features of the anti-CD40 antibodies are incorporated herein by reference.

In one embodiment, the antigen-binding site specifically binds CD73. In one embodiment, the CD73 antigen-binding site comprises the CDRH1, CDRH2, CDR3, CDRL1, CDRL2 and CDRL3, or the V_{H}, or the V_{L} or the V_{H} and V_{L} region from 1E9 (Santa Cruz Biotechnology), AD2, 7G2, 4G4 or from any one of the anti-CD73 antibodies described in WO2017/064043 (including 7H10, 12F9, 15D7, 4B11, 11D9 and 9D2), WO2016/081748 (including 4C3, 7A11, 6E11, 5F8, 4C3, 11F11, 11A6, CD73.4-1, CD73.4-2, CD73.3, 11F11-1, 11F11-2, 11F11, 4C3-1, 4C3-2, 4C3-3, 4D4, 10D2-1, 10D2-2, 11A6, 24H2, 5F8-1, 5F8-2 and 5F8-3), WO2016/131950 (including 11E1, 8C7, 3C12 and 6E1), WO2016/075176 (including MEDI9447, clone 10.3 and clone 2C5) & WO2016/075099 (including CD730004, CD730008, CD7300011, CD730021, CD730042, CD730046, CD730047, CD730068 and CD730069), WO2016/055609 (including 11E1, 6E1, 3C12 and 8C7); the sequences and features of the anti-CD73 antibodies are incorporated herein by reference.

In one embodiment, the antigen-binding site specifically binds CD96. In one embodiment, the CD96 antigen-binding site comprises the CDRH1, CDRH2, CDR3, CDRL1, CDRL2 and CDRL3, or the V_{H}, or the V_{L} or the V_{H} and V_{L} region of 6A6, or NK92.39 (E bioscience), 1C8, 3H8, MAA6359 or from any one of the anti-CD96 antibodies described in WO2008/073316, WO2009/007124, WO2013/184912, WO2014/089169, WO2014/149310 (including antibody 3.3), WO2015/024060 or WO2015/024042, WO2015/024060 (including mAb 3.3); the sequences and features of the anti-CD96 antibodies are incorporated herein by reference.

In one embodiment, the antigen-binding site specifically binds CXCR2. In one embodiment, the CXCR2 antigen-binding site comprises the CDRH1, CDRH2, CDR3, CDRL1, CDRL2 and CDRL3, or the V_{H}, or the V_{L} or the V_{H} and V_{L} region from any one of the anti-CXCR2 antibodies described in WO2015/169811 (including HY29 and HY29GL), WO2014/170317 (including CX2-Mab #1 to #19), WO2012/062713, WO2013/168108 (including 163D2-127D1, 163E3-127D1, 163E3-54B12, 163D2-54B12, 2B2-163E3, 2B2-163D2, 97A9-2B2, 97A9-54B12, 127D1-163D2, 127D1-163E3, 2B2-97A9, 54B12-163D2, 54B12-163E3, 163D2-2B2, 163E3-2B2, 127D1-97A9, 54B12-97A9, 97A9-127D1 and derivatives thereof), WO2009/117706 (including 48311.211, 5E8/CXCR2, clone 19 and derivatives thereof), WO2009/120186 (including RII115, 48311 and derivatives thereof) and WO2002/26249; the sequences and features of the anti-CXCR2 antibodies are incorporated herein by reference.

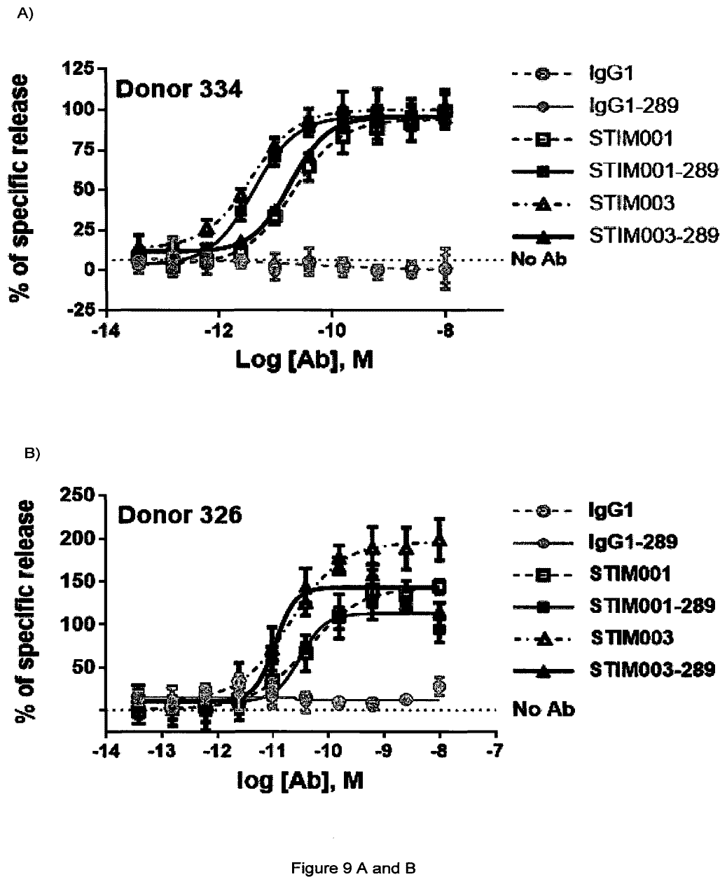

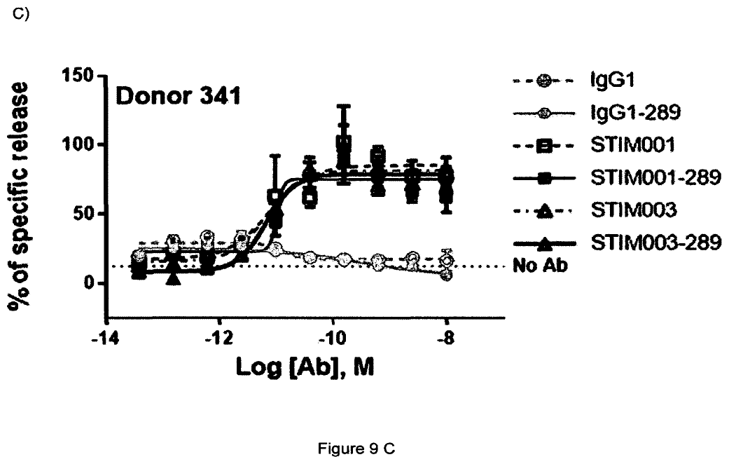

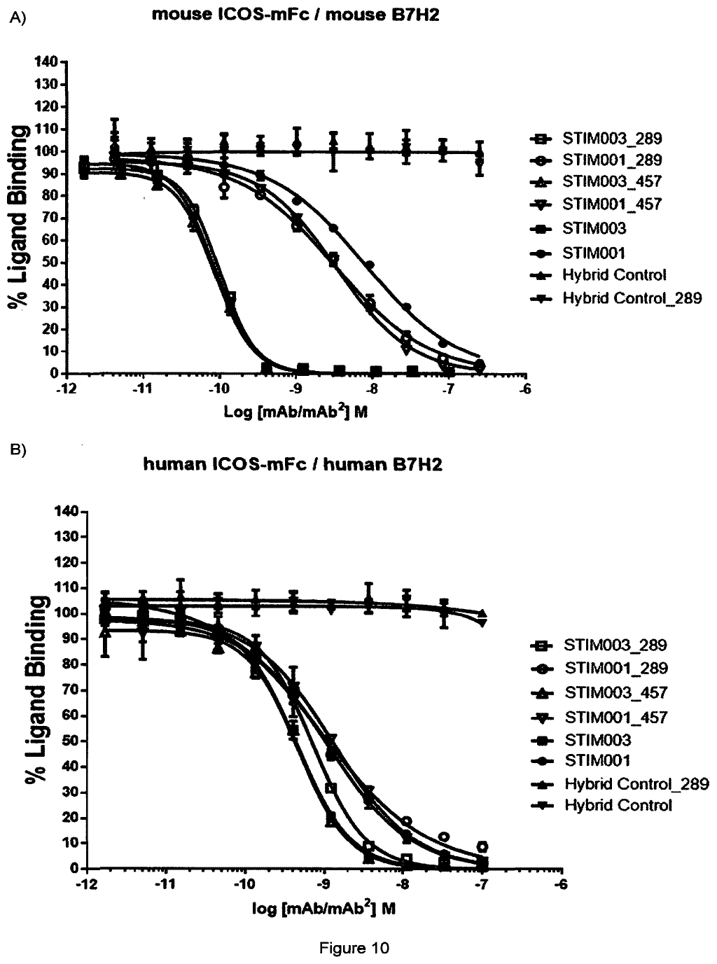

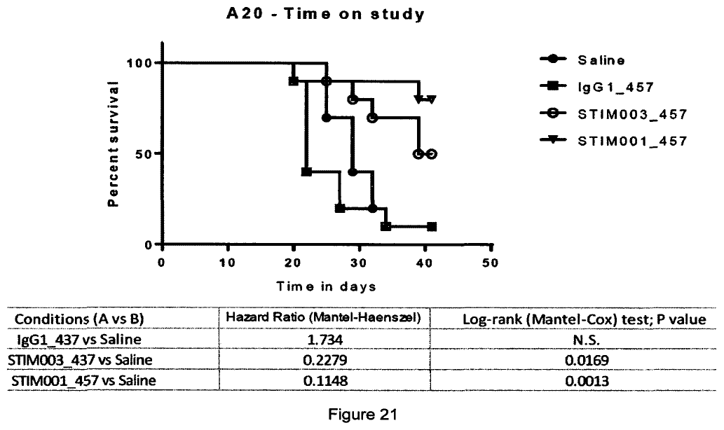

In one embodiment, the antigen-binding site specifically binds CD200. In one embodiment, the CD200 antigen-binding site comprises the CDRH1, CDRH2, CDR3, CDRL1, CDRL2 and CDRL3, or the V_{H}, or the V_{L} or the V_{H} and V_{L} region DX-109, samalizumab/ALXN-6000, TTI-200.7 or from any one of the anti-CD200 antibodies described in WO99/24565 (including M3B5 and the antibodies in Examples 4 and 5), WO02/11762 (including 3B6 and the antibodies in the Examples), WO2004/060295 (US2004/0213783), WO2004/078938 (including scFv-9), WO2006/020266 (U.S. Pat. No. 8,840,885B2, including CG1R3A10, cG2aR3A10, cG2aR3B7, dGIR3A5, dGIR3B5, and dGIR3B10 and the antibodies described in FIGS. 9A-9C, FIGS. 21A and 21B), WO2007/084321 (U.S. Pat. No. 8,709,415B2, including ALXN5200, hB7VH3VL2, C2aB7G1, C2aB7G2/G4, V3V2-G1 and V3V2-G2/G4), WO2009/014745 (including OX90mG2a (FIG. 10), OX90NE and OX90NE-AG), and WO2011/100538 & US2013/0189258 (including Antibody 1 and Antibody 2); the sequences and features of the anti-CD200 antibodies are incorporated herein by reference.

In one embodiment, the antigen-binding site specifically binds CCR4, e.g. human CCR4. In one embodiment, the CCR4 antigen-binding site comprises the CDRH1, CDRH2, CDR3, CDRL1, CDRL2 and CDRL3, or the V_{H}, or the V_{L} or the V_{H} and V_{L} region from mogamulizumab, KM3060 (see Niwa et al., 2004, Cancer Research 64, 2127-2133), and KW-0761 (see Ishida et al., Annals of Oncology 2008, vol 19, supplement 4, 513) or from any one of the anti-CCR4 antibodies described in WO2016/178779 & WO2016/057488 (including mAb2-3, 1-44, 1-49, 2-1 and 2-2), WO2015/179236 (including KW-0761), WO2013/166500 (including mAb1567, c1567, h1567, mAb 1-4 and 2-3 and the antibodies in Examples 6 and 13), WO2012/076883 (including antibodies 208, 306, 308, 406, 501, 503, 601, 603 and 803-Tables 1-9), WO2010/142952 (including 17G, 9E, 11F, 9E10, 9E10J and 9E1D-see Tables 1-16), WO2009/086514 (including mAb1567 and the humanised mAbs in Example 14), WO2005/035582 (including the DG44/CCR4 antibody and the Ms705/CCR4 antibody (FERM BP-8467)), WO2005/053741 & WO01/64754 (U.S. Pat. Nos. 6,989,145B, 7,666,418B, 8,197,814B, 8,632,996B, including KM2160 (FERM BP-10090), KM2760 (FERM deposit BP-7054)), WO2003/018635 (including KM2160, KM8759 (FERM BP-8129) and KM8760 (FERM BP-8130), WO00/42074 (U.S. Pat. Nos. 6,488,930B, 7,138,117B, including 2B10, 10E4, 1G1 and the antibodies deposited as ATCC accession number HB-12624 and HB-12625) and WO00/41724 (U.S. Pat. Nos. 6,881,406B, 6,245,332B, including 1G1 and the antibody deposited under ATCC accession number HB-12624); the sequences and features of the anti-CCR4 antibodies are incorporated herein by reference.

In one embodiment, the antigen-binding site specifically binds CXCL9, e.g. human CXCL9. In one embodiment, the CXCL9 antigen-binding site comprises the CDRH1, CDRH2, CDRH3, CDRL1, CDRL2 and CDRL3, or the V_{H}, or the V_{L} or the V_{H} and V_{L} region from mAb 392-100 or AF392 (R&D Systems).

In one embodiment, the antigen-binding site specifically binds CXCL10. In one embodiment, the CXCL10 antigen-binding site comprises the CDRH1, CDRH2, CDR3, CDRL1, CDRL2 and CDRL3, or the V_{H}, or the V_{L} or the V_{H} and V_{L} region of mAb266 (R & D systems) or from any one of the anti-CXCL10 antibodies described in WO017/8708 (including CR.G (IP-10) (IgG1) (PharMingen) ande IP-10 (IgG) (A.Luster), WO02/15932, WO03/006045, WO2004/082714, WO2004/045525, WO2004/045526, WO2004/101511 (including antibodies in table 1 and AIP12, HuAIP12, MuAIP12, AIP13, HuAIP13, MuAIP13, AIP6, AIP8, AIP14, AIP18, AIP21, AIP22, AIP5 and AIP17), WO2005/060457 (including AIP5, AIP6, AIP8, AIP10, AIP12, AIP13, AIP14, AIP17, AIP18, AIP21, AIP22, AIP32 and AIP36), WO2005/011605, WO2005/023201, WO2005/058815 (including 1D4, 1E1, 2G1, 3C4, 6A5, 6A8, 6B10, 7C10, 8F6, 10A12 and 10A12S13C4), WO2005/084708, WO2006/039819, WO2006/118085, WO2008/047486, WO2008/044824 (including antibodies #124, #31, #28, #43 and #137), WO2008/106200, WO2009/023566, WO2012/149320 (including MSX-1100 and 6A5), WO2014/003742 (including the antibody of Example 14), WO2013/170735, WO2014/189306, WO2015/063187; the sequences and features of the anti-CXCL10 antibodies are incorporated herein by reference.

In one embodiment, the antigen-binding site specifically binds CD155, e.g. human CD155. In one embodiment, the CD155 antigen-binding site comprises the CDRH1, CDRH2, CDRH3, CDRL1, CDRL2 and CDRL3, or the V_{H}, or the V_{L} or the V_{H} and V_{L} region from clone SKII.4 (BioLegend).

In one embodiment, the antigen-binding site specifically binds an immune activator. In one embodiment, the antigen-binding site specifically binds an immune activator selected from CD137, GITR, OX40, CD40, CXCR3 (e.g. agonistic activity against CXCR3), CD3 and ICOS (e.g. agonistic activity against ICOS). In one embodiment, the antigen-binding site specifically binds an immune activator selected from ICOS, CD137, GITR and OX40.

In one embodiment, the antigen-binding site specifically binds CD137, e.g. hCD137. In one embodiment, the CD137 antigen-binding site comprises the CDRH1, CDRH2, CDRH3, CDRL1, CDRL2 and CDRL3, or the V_{H}, or the V_{L} or the V_{H} and V_{L} region from urelumab, BMS-663513, PF-05082566 (Pfizer), 1D8 and 3E1, 4B4 (BioLegend 309809), H4-1BB-M127 (BD Pharmingen 552532), BBK.2 (Thermo Fisher M S621PABX), 145501 (Leinco Technologies B591), the antibody produced by cell line deposited as ATCC No. HB-11248 (U.S. Pat. No. 6,974,863) or XmAb-5592, or from any one of the anti-CD137 antibodies described in WO2017/04945, WO2016/134358, WO2015/179236, WO2012/177788, WO2012/145183, WO2012/032433, WO2009/135019, WO2005/035584, U.S. Pat. No. 6,974,863, WO2004/055513 and WO2004/010947; the sequences and features of the anti-CD137 antibodies are incorporated herein by reference.

In one embodiment, the antigen-binding site specifically binds GITR, e.g. hGITR. In one embodiment, the GITR antigen-binding site comprises the CDRH1, CDRH2, CDR3, CDRL1, CDRL2 and CDRL3, or the V_{H}, or the V_{L} or the V_{H} and V_{L} region from MK4166, TRX518, TRX385, MAB689 (R & D Systems), YGITR765 (Novus Biologicals) or 1D8 (Novus Biologicals), or from any one of the anti-GITR antibodies described in WO2015/187835 (including 28F3, 3C3-1, 3C3-2, 2G6, 8A6, 9G7-1, 9G7-2, 14E3, 19H8-1, 19H8-2, 19D3, 18E10, and 6G10), WO2015/184099 (including 1042-7, 32-15, 1039-45, 1333-21, 231-1039-45, 231-32-15, Hum231 #1, Hum231 #2, m6C8, pab1964, to pab1973, pab1975 to pab1977, pab1979 to pab1981, pab1983, pab2159, pab2160, pab2161 and the antibodies in tables 1 and 2), WO2015/031667 (including antibodies Ab1 to Ab59 in table 1), WO2015/026684 (including an antibody with a CDR sequence of Seq ID 1-66), WO2013/039954 (including, 2155, 1718, 1649, 1362, 954, 827, 698, 706 and antibodies listed in Tables 1 & 3), WO2011/051726 (including antibodies containing CDRs a-f listed on page 17), WO2011/028683 (including antibodies 36E5, 61F6, 61G6, 3D6, 6H6, 1D8, 17F10, 35D8, 49A1, 9E5, 31H6 and antibodies from hybridomas PTA-9889, PTA-9890, PTA-9891, PTA-9892, PTA-9893, PTA-10286, PTA-10287, PTA-10288, PTA-10289, PTA-10290, and PTA-10291), WO2009/009116 (including antibody 2F8), WO2007/133822 (including antibodies listed in Table 1), WO2006/105021 (including 6C8, 2F8, HuN6C8-Agly, HuQ6C8-Gly, and HuQ6C8-Agly), WO2006/050172 & WO2004/084942 (including DTA-1), WO03/006058 (including anti-GITR/TNFRSF18 #AF524), WO2016/054638 (including mAb #1-81, #3-167, #5-139, #7-192, #10-116, #11-126, #12-46, #13-169, #14-182, #15-68 and #17-60), WO2016/196792 (including 6G10, 28F3, 19D3, 18E10, 3C3, 2G6, 8A6, 9G7, 14E3 and 19H8), WO2017/087678 (including 28F3, 19D3, 18E10, 3C3-1, 3C3-2, 2G6, 8A6, 9G7-1, 9G7-2, 14E3, 19H8-1, 19H8-2 and 6G10); the sequences and features of the anti-GITR antibodies are incorporated herein by reference.

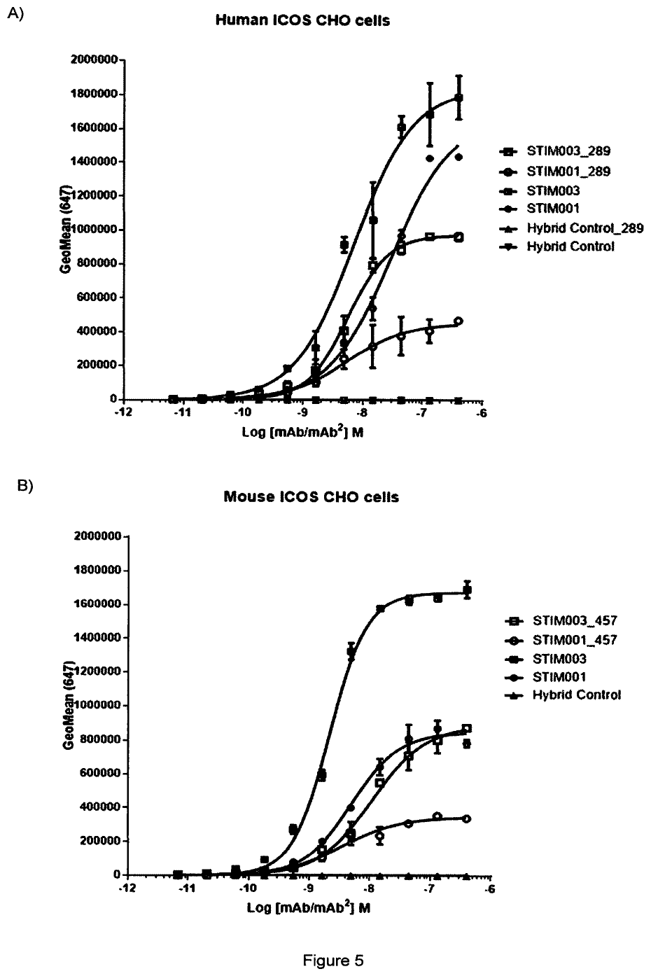

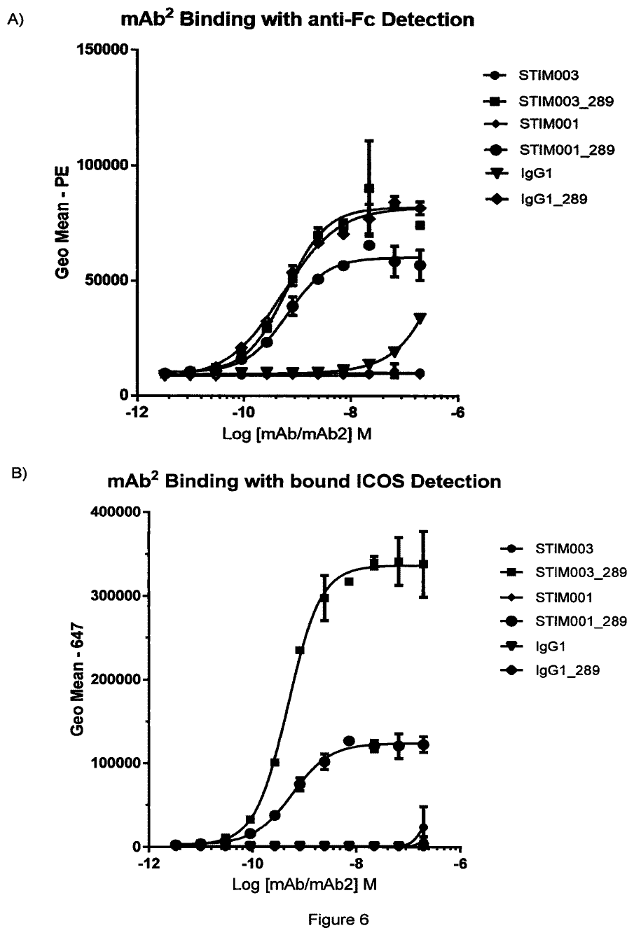

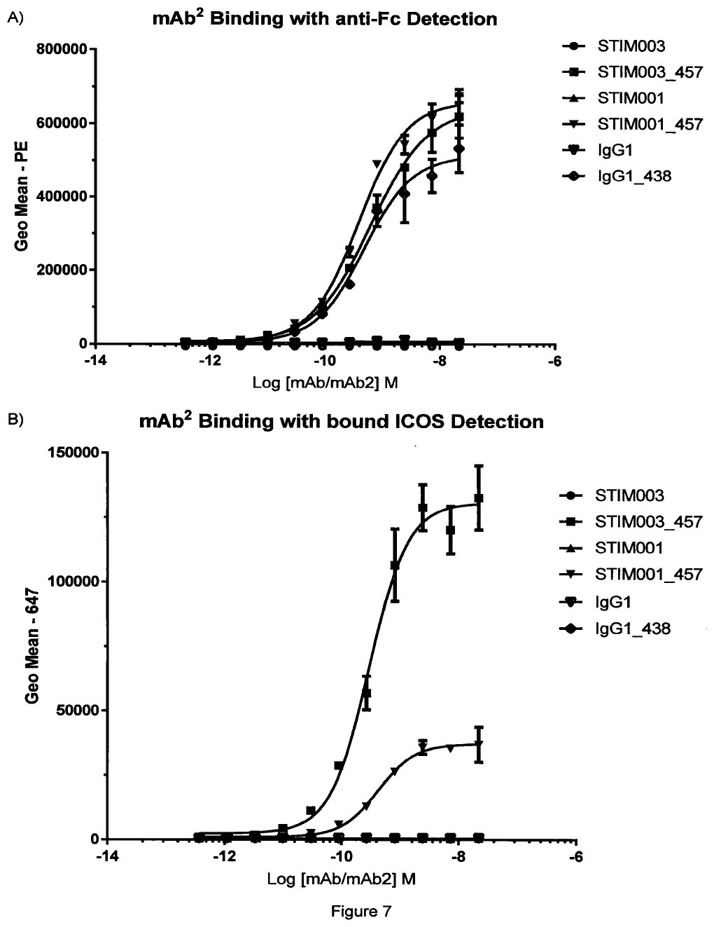

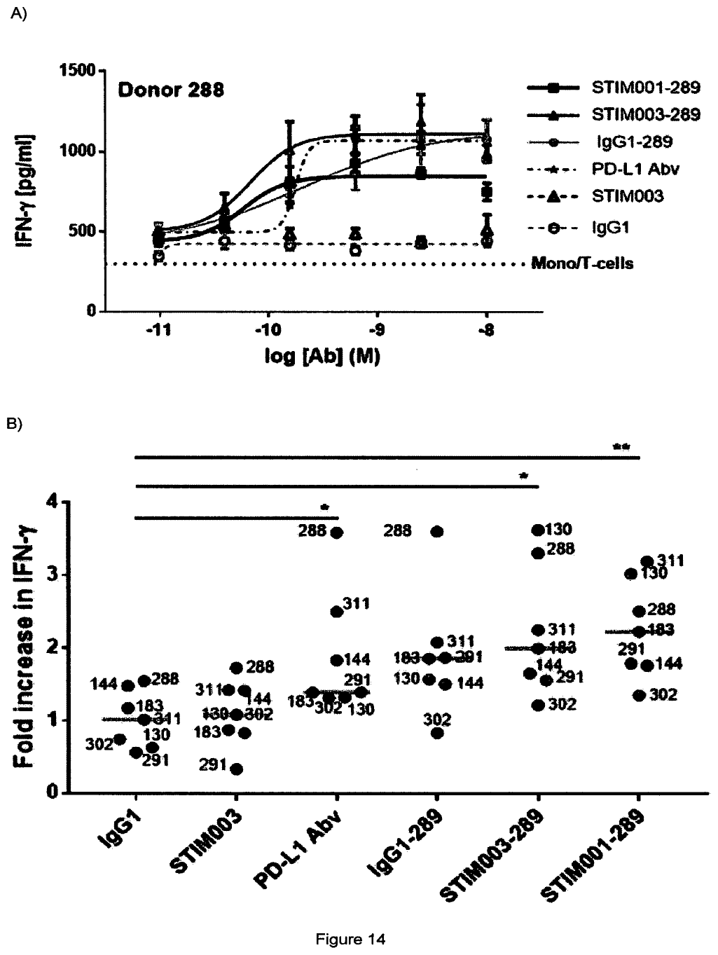

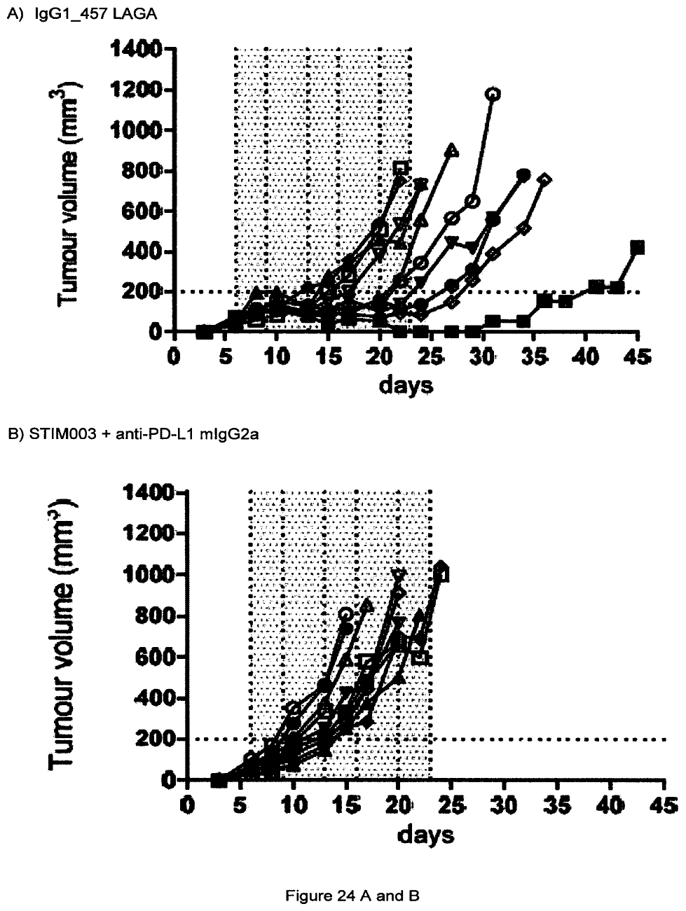

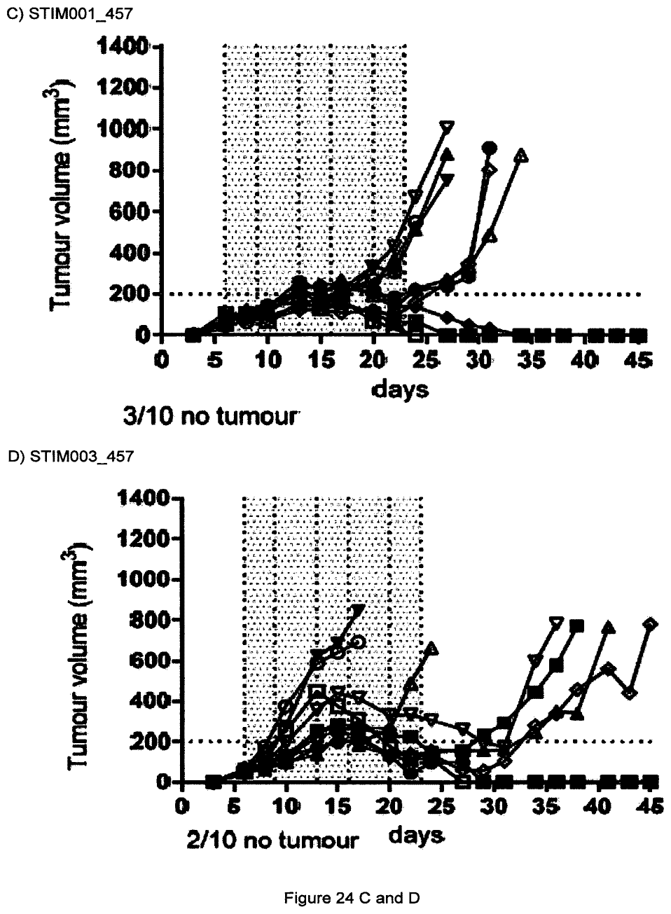

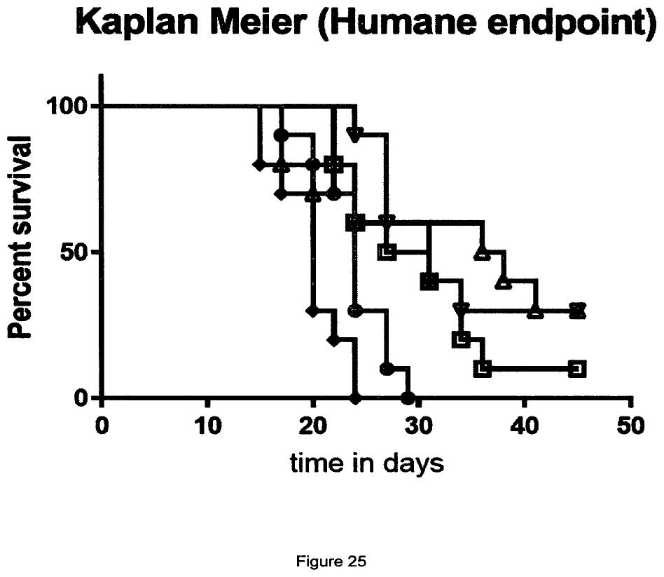

In one embodiment, the antigen-binding site specifically binds OX40, e.g. hOX40. In one embodiment, the OX40 antigen-binding site comprises the CDRH1, CDRH2, CDR3, CDRL1, CDRL2 and CDRL3, or the V_{H}, or the V_{L} or the V_{H} and V_{L} region from GSK3174998, L106 BD (Pharmingen Product #340420), ACT35 (Santa Cruz Biotechnology, Catalog #20073), MOXR0916, MEDI-6469, MEDI-0562, 9B12 (Weinberg, A. D., et al., J Immunother 29, 575-585 (2006)), the humanised anti-OX40 Ab described in Morris et al., Mol Immunol. May 2007; 44(12):3112-3121, or from any one of the anti-OX40 antibodies described in WO2017/077085 (including SAP9, SAP28.2, SAP15.3, SAP29-50, SAP25-29 and SAP29-23 and humanised versions described in Examples 4 and 5), WO2017/063162 (including O3, O19, O21 and the affinity matured version in Example 5-Table 2, including 21 #H28H33, 21 #H65, 21 #H96, 21 #VHnew-L80, 21 #H96-L80), WO2017/050729 (including SP197), WO2017/021912 & WO2017/021910 (including ANTIBODY 106-222, OX86, and the antibodies described in FIGS. 6 and 7), WO2016/200836 & WO2016/200835 (including MOXR0916/1A7.gr1 IgG1), WO2016/196228 (including 3F4, 14B6-1, 14B6-2, 23H3, 18E9, 8B11, 20B3, 20C1, 6E1-1, 6E1-2, 14A2, 14A2-1, 14A2-2, L106, OX40.1, OX40.5, OX40.8, OX40.6, and OX40.16 and OX40.21—FIGS. 1 to 10), WO2016/179517 (including 11D4, pab1949, pab1949-1, pab2044, pab2193-1, Tables 1 to 4), WO2016/057667 (including 9B12 and OX40mAb24), WO2015/153513 (including 3C8, 1D2, 1A7 and their variants described in the sequence listing, including A1A7.gr1 and 3C8.gr.5, the antibodies described in FIG. 1), WO2014/148895 (including ACT35, 12H3, 12H3 (FIG. 25)—and humanised versions VL1H1, VL1VH2, VL1VH3, VL2H1, VL2VH2 and VL2VH3 (FIGS. 43 & 44) and 20E5 (FIG. 24)), WO2013/068563 (including A26 [FIG. 2]), WO2013/038191 (including ACT35, 12H3 and 12H3), WO2013/028231 (including 119-122, 119-43-1, 106-222 and the antibodies in Table 1), WO2013/008171 (including 2F8, 1D4 and their derivatives, including VH6/VL9, and the antibodies in FIGS. 4 and 5 and tables 6 and 7), WO2012/027328 (including 119-122, 119-43-1, Hu106 and Hu106-222), WO2010/096418 (including A26), WO2008/106116 (including the antibodies in Tables 1 and 2, and A10 (inc A10A-F), B66—FIG. 14—B2, B24, B36, B37, and B39) and WO2007/062245 (including 112V8 (ATCC No. PTA-7219), 112Y55 (ATCC No. PTA-7220), 112Y131 (ATCC No. PTA-7218), 112F32 (ATCC No. PTA-7217) and 112Z5 (ATCC No. PTA-7216); the sequences and features of the anti-OX40 antibodies are incorporated herein by reference.

In one embodiment, the antigen-binding site specifically binds CXCR3, e.g. CXCR3. In one embodiment, the CXCR3 antigen-binding site comprises the CDRH1, CDRH2, CDRH3, CDRL1, CDRL2 and CDRL3, or the V_{H}, or the V_{L} or the V_{H} and V_{L} region from GSK3174998 or from any one of the anti-CXCR3 antibodies described in WO2016/200836, WO2016/200835, WO2016/196228, WO2016/179517, WO2016/057667, WO2015/153513, WO2014/148895, WO2013/068563, WO2013/038191, WO2013/028231, WO2013/008171, WO2012/027328, WO2010/096418, WO2011/073180, WO2008/106116 and WO2007/062245; the sequences and features of the anti-CXCR3 antibodies are incorporated herein by reference.

In one embodiment, the antigen-binding site specifically binds CD27, e.g. hCD27. In one embodiment, the CD27 antigen-binding site comprises the CDRH1, CDRH2, CDRH3, CDRL1, CDRL2 and CDRL3, or the V_{H}, or the V_{L} or the V_{H} and V_{L} region from any one of the anti-CD27 antibodies described in WO2016/145085 (including 1F5), WO2015/016718 (including hCD27.15 and 1F5), WO2014/140374 (including 2F2, 5F24, 5F32, 10F13, 10F31, 11F26, 1052 to 015, F2A4B2 and their derivatives, including hz5F24VH+V5Q, hz5F24VL+K45Q), WO2013/138586 (including C2177, C2186, C2191, and C2192 and the derivatives in Examples 8 to 12, and tables 7 to 42), WO2012/004367 (including hCD27.15/ATCC number PTA-11008), WO2011/130434 (including 1G5, 1H8, 3H12, 3H8, 2G9, 1F5, 3A10, 2C2, ms 1A4, ms 9F4 and ms M-T271), WO2011/081164 & WO2010/001908 (including KM4027, KM4028, KM4026, KM4030, KM4032 and derivatives thereof), WO2008/051424 (including LG3A10 and AT124-1); the sequences and features of the anti-CD27 antibodies are incorporated herein by reference.

In one embodiment, the antigen-binding site specifically binds CD3, e.g. hCD3. In one embodiment, the CD3 antigen-binding site comprises the CDRH1, CDRH2, CDRH3, CDRL1, CDRL2 and CDRL3, or the V_{H}, or the V_{L} or the V_{H} and V_{L} region from OKT3 antibody, otelixizumab, teplizumab or visilizumab, or from any one of the anti-CD3 antibodies described in WO2017/010874, WO2017/009442, WO2016/204966, WO2016/180721, WO2016/179003, WO2016/116626, WO2016/014974, WO2015/104346, WO2015/095392, WO2015/001085, WO2014/047231, WO2013/188693, WO2013/186613, WO2013/158856, WO2012/173819, WO2012/162067, WO2005/118635, WO2004/108158, WO2004/052397, WO2004/024771, WO01/51644, WO00/05268, WO97/44362, WO93/19196, WO92/06193 and WO91/09968; the sequences and features of the anti-CD3 antibodies are incorporated herein by reference.

### Configurations Relating to PD-L1 Antibodies

Disclosed herein are antibodies and antigen binding fragments thereof that specifically bind to PD-L1. In one embodiment, the antibody or antigen binding fragment thereof specifically binds to surface expressed PD-L1.

In a first configuration, there is provided an antibody or a fragment thereof, that specifically binds to hPD-L1 as defined by Seq ID No:1, and competes for binding to said hPD-L1 with the antibody 1D05, wherein the antibody or fragment comprises a V_{H} domain which comprises a CDRH3 comprising the motif X₁GSGX₂YGX₃X₄FD, wherein X₁, X₂ and X₃ are independently any amino acid, and X₄ is either present or absent, and if present, may be any amino acid (SEQ ID NO: 563).

In a second configuration, there is provided an antibody or a fragment thereof which specifically binds to hPD-L1, and competes for binding to said hPD-L1 with the antibody 1D05, wherein the antibody or fragment comprises a V_{H} domain which comprises the CDRH3 sequence of SEQ ID NO:29 or 32, or the CDRH3 sequence of SEQ ID NO:29 or 32 comprising 6 or fewer amino acid substitutions.

In a third configuration, there is provided an antibody or fragment thereof which specifically binds to an epitope that is identical to an epitope to which the antibody 1D05 specifically binds.

In a fourth configuration, there is provided an antibody or fragment thereof which competes for binding to hPD-L1 with the antibody 1D05.

In a fifth configuration, there is provided a bispecific antibody or fusion protein comprising an antibody or fragment thereof as defined in any other configuration, embodiment or concept.

In a sixth configuration, there is provided an antibody or fragment as defined in any other configuration, embodiment or concept for use in treating or preventing a hPD-L1-mediated disease or condition.

In a seventh configuration, there is provided the use of an antibody or fragment as defined in any other configuration, embodiment or concept in the manufacture of a medicament for administration to a human for treating or preventing a hPD-L1 mediated disease or condition in the human.

In an eighth configuration, there is provided a method of treating or preventing a hPD-L1 mediated disease or condition in a human, comprising administering to said human a therapeutically effective amount of an antibody or fragment as defined in any other configuration, embodiment or concept, wherein the hPD-L1 mediated disease or condition is thereby treated or prevented.

In a ninth configuration, there is provided a pharmaceutical composition comprising an antibody of fragment as defined in any other configuration, embodiment or concept and a pharmaceutically acceptable excipient, diluent or carrier.

In a tenth configuration, there is provided a kit comprising a pharmaceutical composition comprising an antibody of fragment as defined in any other configuration, embodiment or concept and a pharmaceutically acceptable excipient, diluent or carrier.

In an eleventh configuration, there is provided a method of modulating PD-1/PD-L1 interaction in a patient, comprising administering an effective amount of an antibody or fragment as defined in any other configuration, embodiment or concept to said patient.

In a twelfth configuration, there is provided a method of inhibiting PD-L1 activity in a patient, comprising administering an effective amount of an antibody or fragment as defined in any other configuration, embodiment or concept to said patient.

In a thirteenth configuration, there is provided a method of treating a proliferative disease in an animal (e.g. a human), comprising administering an effective amount of an antibody or fragment as defined in any other configuration, embodiment or concept to said patient.

In a fourteenth configuration, there is provided a method of detecting PD-L1 expression in a sample, comprising contacting the sample with an antibody or fragment as defined in any other configuration, embodiment or concept.

In a fifteenth configuration, there is provided a method comprising contacting a biological sample with an antibody or fragment as defined in any other configuration, embodiment or concept to form a complex with PD-L1 present in the sample and measuring the presence, absence or level of the complex in the biological sample.

In a sixteenth configuration, there is provided a method of detecting PD-L1 expression in a sample, comprising contacting the sample with an antibody or fragment as defined in any other configuration, embodiment or concept.

In a seventeenth configuration, there is provided a method comprising contacting a biological sample with an antibody or fragment as defined in any other configuration, embodiment or concept to form a complex with PD-L1 present in the sample and measuring the presence, absence or level of the complex in the biological sample.

In a eighteenth configuration, there is provided a method for identifying binding partners for PD-L1, the method comprising immunoprecipitating an intact protein complex comprising PD-L1 using an antibody or fragment as defined in any other configuration, embodiment or concept.

In a nineteenth configuration, there is provided a method of diagnosing a disease in a human subject associated with altered PD-L1 expression comprising the steps of contacting a biological sample from the human subject with an antibody as defined in other configuration, embodiment or concept to form a complex between the antibody and PD-L1 present in the sample; and detecting the amount of the complex.

In a twentieth configuration, there is provided a nucleic acid that encodes the CDRH3 of an antibody or fragment as defined in any other configuration, embodiment or concept. In a twenty-first configuration, there is provided a nucleic acid that encodes a VH domain and/or a VL domain of an antibody or fragment as defined in any other configuration, embodiment or concept.

In a twenty-second configuration, there is provided a vector comprising the nucleic acid of any other configuration, embodiment or concept; optionally wherein the vector is a CHO or HEK293 vector.

In a twenty-third configuration, there is provided a host comprising the nucleic acid of any other configuration, embodiment or concept or the vector of any other configuration, embodiment or concept.

### ICOS

“ICOS” or “the ICOS receptor” referred to herein may be human ICOS, unless the context dictates otherwise. Sequences of human, cynomolgus and mouse ICOS are shown in the appended sequence listing, and are available from NCBI as human NCBI ID: NP_036224.1, mouse NCBI ID: NP_059508.2 and cynomolgus GenBank ID: EHH55098.1.

The ICOS ligand (ICOSL, also known as B7-H2) is a cell surface expressed molecule that binds to the ICOS receptor [23]. This intercellular ligand-receptor interaction promotes multimerisation of ICOS on the T cell surface, activating the receptor and stimulating downstream signalling in the T cell. In effector T cells, this receptor activation stimulates the effector T cell response.

### Antibodies to ICOS

Anti-ICOS antibodies may act as agonists of ICOS, mimicking and even surpassing the stimulatory effect of the native ICOS ligand on the receptor. Such agonism may result from ability of the antibody to promote multimerisation of ICOS on the T cell. One mechanism for this is where the antibodies form intercellular bridges between ICOS on the T cell surface and receptors on an adjacent cell (e.g., B cell, antigen-presenting cell, or other immune cell), such as Fc receptors and/or receptors to which the multi-specific antibody binds. Another mechanism is where antibodies having multiple (e.g., two) antigen-binding sites (e.g., two VH-VL domain pairs) bridge multiple ICOS receptor molecules and so promote multimerisation. A combination of these mechanisms may occur.

A bispecific antibody combining both ICOS agonism with PD-L1 antagonism may act via its PD-L1 binding arm (e.g., Fcab) to inhibit the negative co-regulatory signals generated by PD-L1 expressed on APCs, myeloid-derived suppressor cells (MDSC) or tumour cells, and may instead deliver a positive agonistic signal via its ICOS binding arm (e.g., Fab). See FIG. 1.

An antibody to ICOS that acts to increase effector T cell activity represents a therapeutic approach in immunooncology and in other medical contexts where a CD8+ T cell response is beneficial. In many diseases and conditions involving an immune component, a balance exists between effector T cells (TEff) which exert the CD8+ T cell immune response, and regulatory T cells (TReg) which suppress that immune response by downregulating TEffs. The present invention relates to antibodies that modulate this TEff/TReg balance in favour of effector T cell activity. Antibodies that trigger the depletion of ICOS highly positive regulatory T cells would relieve the suppression of TEffs, and thus have a net effect of promoting the effector T cell response. An additional or complementary mechanism for an anti-ICOS antibody is via agonistic activity at the ICOS receptor level, to stimulate the effector T cell response.

The relative expression of ICOS on effector T cells (TEff) compared with regulatory T cells (TReg), and the relative activities of these cell populations, will influence the overall effect of an anti-ICOS antibody in vivo. An envisaged mode of action combines agonism of effector T cells with depletion of ICOS positive regulatory T cells. Differential and even opposing effects on these two different T cell populations may be achievable due to their different levels of ICOS expression. Dual-engineering of the variable and constant regions respectively of an anti-ICOS antibody can provide a molecule that exerts a net positive effect on effector T cell response by affecting the CD8/TReg ratio. An antigen-binding domain of an agonist antibody, which activates the ICOS receptor, may be combined with an antibody constant (Fc) region that promotes downregulation and/or clearance of highly expressing cells to which the antibody is bound. An effector positive constant region may be used to recruit cellular effector functions against the target cells (TRegs), e.g., to promote antibody-dependent cell-mediated cytotoxicity (ADCC) or antibody dependent cell phagocytosis (ADCP). A bispecific antibody binding to ICOS and PD-L1 may trigger ADCC of PD-L1+ immunosuppressive cells (e.g., MDSC, tumour cells) and ICOS+ immunosuppressive cells (Tregs). The antibody may thus act both to promote effector T cell activation and to downregulate immunosuppressive T Regulatory cells. Since ICOS is more highly expressed on TRegs than on TEffs, a therapeutic balance may be achieved whereby Teff function is promoted while TRegs are depleted, resulting in a net increase in the T cell immune response (e.g, anti-tumour response or other therapeutically beneficial T cell response).

The ICOS binding site of multi-specific antibodies described herein may bind human ICOS. The antibodies target the ICOS extracellular domain and thereby bind to T cells expressing ICOS. Examples are provided of antibodies that have been designed to have an agonistic effect on ICOS, thus enhancing the function of effector T cells, as indicated by an ability to increase IFNγ expression and secretion. As noted, anti-ICOS antibodies may also be engineered to deplete cells to which they bind, which should have the effect of preferentially downregulating regulatory T cells, lifting the suppressive effect of these cells on the effector T cell response and thus promoting the effector T cell response overall. Regardless of their mechanism of action, it is demonstrated empirically herein that anti-ICOS antibodies stimulate T cell response and have anti-tumour effects in vivo, as shown in the Examples. Through selection of appropriate antibody formats such as those including constant regions with a desired level of Fc effector function, or absence of such effector function where appropriate, the anti-ICOS antibodies may be tailored for use in a variety of medical contexts including treatment of diseases and conditions in which an effector T cell response is beneficial and/or where suppression of regulatory T cells is desired.

ICOS antibodies are provided herein. The ICOS antibodies may be any of those described in GB patent application 1620414.1 (filed 1 Dec. 2016), the sequences of the anti-ICOS antibodies disclosed therein are incorporated herein by reference.

STIM001 has a heavy chain variable region (V_{H}) amino acid sequence of Seq ID No: 366, comprising the CDRH1 amino acid sequence of Seq ID No:363, the CDRH2 amino acid sequence of Seq ID No:364, and the CDRH3 amino acid sequence of Seq ID No:365. The heavy chain nucleic acid sequence of the V_{H} domain is Seq ID No:367. STIM001 has a light chain variable region (V_{L}) amino acid sequence of Seq ID No:373, comprising the CDRL1 amino acid sequence of Seq ID No:370, the CDRL2 amino acid sequence of Seq ID No: 371, and the CDRL3 amino acid sequence of Seq ID No:372. The light chain nucleic acid sequence of the V_{L} domain is Seq ID No:374. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:368 (heavy chain nucleic acid sequence Seq ID No:369). A full length light chain amino acid sequence is Seq ID No:375 (light chain nucleic acid sequence Seq ID No: 376).

STIM002 has a heavy chain variable region (V_{H}) amino acid sequence of Seq ID No: 380, comprising the CDRH1 amino acid sequence of Seq ID No:377, the CDRH2 amino acid sequence of Seq ID No:378, and the CDRH3 amino acid sequence of Seq ID No:379. The heavy chain nucleic acid sequence of the V_{H} domain is Seq ID No:381. STIM002 has a light chain variable region (V_{L}) amino acid sequence of Seq ID No:387, comprising the CDRL1 amino acid sequence of Seq ID No:384, the CDRL2 amino acid sequence of Seq ID No: 385, and the CDRL3 amino acid sequence of Seq ID No:386. The light chain nucleic acid sequence of the VL domain is Seq ID No:388 or Seq ID No:519. The V_{H} domain may be combined with any of the heavy chain constant region sequences described herein, e.g. Seq ID No: 193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No: 530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:382 (heavy chain nucleic acid sequence Seq ID No: 383). A full length light chain amino acid sequence is Seq ID No:389 (light chain nucleic acid sequence Seq ID No:390 or Seq ID NO:520).

STIM002-B has a heavy chain variable region (V_{H}) amino acid sequence of Seq ID No: 394, comprising the CDRH1 amino acid sequence of Seq ID No:391, the CDRH2 amino acid sequence of Seq ID No:392, and the CDRH3 amino acid sequence of Seq ID No:393. The heavy chain nucleic acid sequence of the V_{H} domain is Seq ID No:395. STIM002-B has a light chain variable region (V_{L}) amino acid sequence of Seq ID No:401, comprising the CDRL1 amino acid sequence of Seq ID No:398, the CDRL2 amino acid sequence of Seq ID No: 399, and the CDRL3 amino acid sequence of Seq ID No:400. The light chain nucleic acid sequence of the VL domain is Seq ID No:402. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:396 (heavy chain nucleic acid sequence Seq ID No:397). A full length light chain amino acid sequence is Seq ID No:403 (light chain nucleic acid sequence Seq ID No: 404).

STIM003 has a heavy chain variable region (V_{H}) amino acid sequence of Seq ID No:408, comprising the CDRH1 amino acid sequence of Seq ID No:405, the CDRH2 amino acid sequence of Seq ID No:406, and the CDRH3 amino acid sequence of Seq ID No:407. The heavy chain nucleic acid sequence of the V_{H} domain is Seq ID No:409 or Seq ID No:521. STIM003 has a light chain variable region (V_{L}) amino acid sequence of Seq ID No:415, comprising the CDRL1 amino acid sequence of Seq ID No:412, the CDRL2 amino acid sequence of Seq ID No:413, and the CDRL3 amino acid sequence of Seq ID No:414. The light chain nucleic acid sequence of the V_{L} domain is Seq ID No:4416. The V_{H} domain may be combined with any of the heavy chain constant region sequences described herein, e.g. Seq ID No:193, Seq ID No:195, Seq ID No:197, Seq ID No:199, Seq ID No:201, Seq ID No: 203, Seq ID No:205, Seq ID No:340, Seq ID No:524, Seq ID No:526, Seq ID No:528, Seq ID No: 530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:410 (heavy chain nucleic acid sequence Seq ID No: 411 or Seq ID No:522). A full length light chain amino acid sequence is Seq ID No:417 (light chain nucleic acid sequence Seq ID No:418). STIM003 comprises a V_{H} domain having HCDR1, HCDR2, and HCDR3 sequences wherein HCDR1 is SEQ ID NO: 405, optionally comprising a conservative substitution at residue 28; HCDR2 is SEQ ID NO: 406, optionally comprising a substitution at residue 59, residue 63 and/or residue 64; and HCDR3 is SEQ ID NO: 407, optionally comprising a substitution at residue 108, residue 109 and/or residue 112, and comprises a VL domain having LCDR1, LCDR2, and LCDR3 sequences wherein LCDR1 is SEQ ID NO: 412, optionally comprising a substitution at residue 36, LCDR2 is SEQ ID NO: 413, and LCDR3 is the SEQ ID NO 414, optionally comprising a substitution at residue 108 or residue 109, and the residue numbering is according to Kabat. The CDR sequence of SEQ ID NO: 405 has a conservative substitution at residue 28 of HCDR1 of V28F. The CDR sequence of SEQ ID NO: 406 has a conservative substitution at residue 59 of HCDR2 of N59I, wherein the substitution at residue 63 of HCDR2 is G63D, and/or wherein the substitution at residue 64 of HCDR2 is D64N. The CDR sequence of SEQ ID NO: 407 has a conservative substitution at residue 108 of HCDR3 is F108Y, wherein the substitution at residue 109 of HCDR3 is Y109F, and/or wherein the substitution at residue 112 of HCDR3 is H112N. The CDR sequence of SEQ ID NO: 412 has a conservative substitution at residue 36 of LCDR1 is R36S. The CDR sequence of SEQ ID NO: 414 has a conservative substitution at residue 108 of LCDR3 is D108G and/or wherein the substitution at residue 109 of LCDR3 is M109N.

STIM004 has a heavy chain variable region (V_{H}) amino acid sequence of Seq ID No: 422, comprising the CDRH1 amino acid sequence of Seq ID No:419, the CDRH2 amino acid sequence of Seq ID No:420, and the CDRH3 amino acid sequence of Seq ID No:421. The heavy chain nucleic acid sequence of the V_{H} domain is Seq ID No:423. STIM004 has a light chain variable region (V_{L}) amino acid sequence of Seq ID No:429, comprising the CDRL1 amino acid sequence of Seq ID No:426, the CDRL2 amino acid sequence of Seq ID No: 427, and the CDRL3 amino acid sequence of Seq ID No:428. The light chain nucleic acid sequence of the VL domain is Seq ID No:430 or Seq ID No:431. The V_{H} domain may be combined with any of the heavy chain constant region sequences described herein, e.g. Seq ID No: 193, Seq ID No:195, Seq ID No:197, Seq ID No:199, Seq ID No:201, Seq ID No:203, Seq ID No:205, Seq ID No:340, Seq ID No:524, Seq ID No:526, Seq ID No:528, Seq ID No: 530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:424 (heavy chain nucleic acid sequence Seq ID No: 425). A full length light chain amino acid sequence is Seq ID No:432 (light chain nucleic acid sequence Seq ID No:433 or Seq ID no: 434).

STIM005 has a heavy chain variable region (V_{H}) amino acid sequence of Seq ID No: 438, comprising the CDRH1 amino acid sequence of Seq ID No:435, the CDRH2 amino acid sequence of Seq ID No:436, and the CDRH3 amino acid sequence of Seq ID No:437. The heavy chain nucleic acid sequence of the V_{H} domain is Seq ID No:439. STIM005 has a light chain variable region (V_{L}) amino acid sequence of Seq ID No:445, comprising the CDRL1 amino acid sequence of Seq ID No:442, the CDRL2 amino acid sequence of Seq ID No: 443, and the CDRL3 amino acid sequence of Seq ID No:444. The light chain nucleic acid sequence of the V_{L} domain is Seq ID No:446. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:440 (heavy chain nucleic acid sequence Seq ID No:441). A full length light chain amino acid sequence is Seq ID No:447 (light chain nucleic acid sequence Seq ID No: 448).

STIM006 has a heavy chain variable region (V_{H}) amino acid sequence of Seq ID No: 452, comprising the CDRH1 amino acid sequence of Seq ID No:449, the CDRH2 amino acid sequence of Seq ID No:450, and the CDRH3 amino acid sequence of Seq ID No:451. The heavy chain nucleic acid sequence of the V_{H} domain is Seq ID No:453. STIM006 has a light chain variable region (V_{L}) amino acid sequence of Seq ID No:459, comprising the CDRL1 amino acid sequence of Seq ID No:456, the CDRL2 amino acid sequence of Seq ID No: 457, and the CDRL3 amino acid sequence of Seq ID No:458. The light chain nucleic acid sequence of the VL domain is Seq ID No:460. The V_{H} domain may be combined with any of the heavy chain constant region sequences described herein, e.g. Seq ID No:193, Seq ID No: 195, Seq ID No:197, Seq ID No: 199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:454 (heavy chain nucleic acid sequence Seq ID No:455). A full length light chain amino acid sequence is Seq ID No:461 (light chain nucleic acid sequence Seq ID No: 462).

STIM007 has a heavy chain variable region (V_{H}) amino acid sequence of Seq ID No: 466, comprising the CDRH1 amino acid sequence of Seq ID No:463, the CDRH2 amino acid sequence of Seq ID No:464, and the CDRH3 amino acid sequence of Seq ID No:465. The heavy chain nucleic acid sequence of the V_{H} domain is Seq ID No:467. STIM007 has a light chain variable region (V_{L}) amino acid sequence of Seq ID No:473, comprising the CDRL1 amino acid sequence of Seq ID No:470, the CDRL2 amino acid sequence of Seq ID No: 471, and the CDRL3 amino acid sequence of Seq ID No:472. The light chain nucleic acid sequence of the VL domain is Seq ID No:474. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:468 (heavy chain nucleic acid sequence Seq ID No:469). A full length light chain amino acid sequence is Seq ID No:475 (light chain nucleic acid sequence Seq ID No: 476).

STIM008 has a heavy chain variable region (V_{H}) amino acid sequence of Seq ID No: 480, comprising the CDRH1 amino acid sequence of Seq ID No:477, the CDRH2 amino acid sequence of Seq ID No:478, and the CDRH3 amino acid sequence of Seq ID No:479. The heavy chain nucleic acid sequence of the V_{H} domain is Seq ID No:481. STIM008 has a light chain variable region (V_{L}) amino acid sequence of Seq ID No:487, comprising the CDRL1 amino acid sequence of Seq ID No:484, the CDRL2 amino acid sequence of Seq ID No: 485, and the CDRL3 amino acid sequence of Seq ID No:486. The light chain nucleic acid sequence of the VL domain is Seq ID No:488. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The VL domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:482 (heavy chain nucleic acid sequence Seq ID No:483). A full length light chain amino acid sequence is Seq ID No:489 (light chain nucleic acid sequence Seq ID No: 490).

STIM009 has a heavy chain variable region (V_{H}) amino acid sequence of Seq ID No: 494, comprising the CDRH1 amino acid sequence of Seq ID No:491, the CDRH2 amino acid sequence of Seq ID No:492, and the CDRH3 amino acid sequence of Seq ID No:493. The heavy chain nucleic acid sequence of the V_{H} domain is Seq ID No:495. STIM009 has a light chain variable region (V_{L}) amino acid sequence of Seq ID No:501, comprising the CDRL1 amino acid sequence of Seq ID No:498, the CDRL2 amino acid sequence of Seq ID No: 499, and the CDRL3 amino acid sequence of Seq ID No:500. The light chain nucleic acid sequence of the V_{L} domain is Seq ID No:502. The V_{H} domain may be combined with any of the heavy chain constant region sequences described herein, e.g. Seq ID No:193, Seq ID No: 195, Seq ID No:197, Seq ID No:199, Seq ID No:201, Seq ID No:203, Seq ID No:205, Seq ID No: 340, Seq ID No:524, Seq ID No:526, Seq ID No:528, Seq ID No:530, Seq ID No:532 or Seq ID No:534. The V_{L} domain may be combined with any of the light chain constant region sequences described herein, e.g. Seq ID Nos: 207, 209, 211, 213, 215, 217, 219, 221, 223, 225, 227, 229, 231, 233, 235, 237, 536 and 538. A full length heavy chain amino acid sequence is Seq ID No:496 (heavy chain nucleic acid sequence Seq ID No:497). A full length light chain amino acid sequence is Seq ID No:503 (light chain nucleic acid sequence Seq ID No: 504).

Antibodies STIM001-009 are described in more detail in GB patent application 1620414.1 (filed 1 Dec. 2016), the contents of which are incorporated herein by reference. ICOS antibodies may also be described as in the following numbered sentences below:

Sentence 1. An isolated antibody that binds the extracellular domain of human and/or mouse ICOS, comprising:
an antibody VH domain comprising complementarity determining regions (CDRs) HCDR1, HCDR2 and HCDR3, and
an antibody VL domain comprising complementarity determining regions LCDR1, LCDR2 and LCDR3, wherein
   HCDR1 is the HCDR1 of STIM001, STIM002, STIM002-B, STIM003, STIM004, STIM005, STIM006, STIM007, STIM008 or STIM009, or comprises that HCDR1 with 1, 2, 3, 4 or 5 amino acid alterations,
   HCDR2 is the HCDR2 of STIM001, STIM002, STIM002-B, STIM003, STIM004, STIM005, STIM006, STIM007, STIM008 or STIM009, or comprises that HCDR2 with 1, 2, 3, 4 or 5 amino acid alterations, and/or
   HCDR3 is the HCDR3 of STIM001, STIM002, STIM002-B, STIM003, STIM004, STIM005, STIM006, STIM007, STIM008 or STIM009 or comprises that HCDR3 with 1, 2, 3, 4 or 5 amino acid alterations.

Sentence 2. An antibody according to sentence 1, wherein the antibody heavy chain CDRs are those of STIM001, STIM002, STIM002-B, STIM003, STIM004, STIM005, STIM006, STIM007, STIM008 or STIM009 or comprise the STIM001, STIM002, STIM002-B, STIM003, STIM004, STIM005, STIM006, STIM007, STIM008 or STIM009 heavy chain CDRs with 1, 2, 3, 4 or 5 amino acid alterations.

Sentence 3. An antibody according to sentence 2, wherein the antibody VH domain has the heavy chain CDRs of STIM003.

Sentence 4. An isolated antibody that binds the extracellular domain of human and/or mouse ICOS, comprising:
an antibody VH domain comprising complementarity determining regions HCDR1, HCDR2 and HCDR3, and
an antibody VL domain comprising complementarity determining regions LCDR1, LCDR2 and LCDR3,
   wherein LCDR1 is the LCDR1 of STIM001, STIM002, STIM002-B, STIM003, STIM004 STIM005, STIM006, STIM007, STIM008 or STIM009, or comprises that LCDR1 with 1, 2, 3, 4 or 5 amino acid alterations,
   LCDR2 is the LCDR2 of STIM001, STIM002, STIM002-B, STIM003, STIM004, STIM005, STIM006, STIM007, STIM008 or STIM009, or comprises that LCDR2 with 1, 2, 3, 4 or 5 amino acid alterations, and/or
   LCDR3 is the LCDR3 of STIM001, STIM002, STIM002-B, STIM003, STIM004, STIM005, STIM006, STIM007, STIM008 or STIM009 or comprises that LCDR3 with 1, 2, 3, 4 or 5 amino acid alterations.

Sentence 5. An antibody according to any preceding sentence, wherein the antibody light chain CDRs are those of STIM001, STIM002, STIM002-B, STIM003, STIM004, STIM005, STIM006, STIM007, STIM008 or STIM009, or comprise the STIM001, STIM002, STIM002-B, STIM003, STIM004, STIM005, STIM006, STIM007, STIM008 or STIM009 light chain CDRs with 1, 2, 3, 4 or 5 amino acid alterations.

Sentence 6. An antibody according to sentence 5, wherein the antibody VL domain has the light chain CDRs of STIM003.

Sentence 7. An antibody according to any of the preceding sentences, comprising VH and/or VL domain framework regions of human germline gene segment sequences.

Sentence 8. An antibody according to any of the preceding sentences, comprising a VH domain which
(i) is derived from recombination of a human heavy chain V gene segment, a human heavy chain D gene segment and a human heavy chain J gene segment, wherein
   the V segment is IGHV1-18 (e.g., V1-18*01), IGVH3-20 (e.g. V3-20*d01), IGVH3-11 (e.g, V3-11*01) or IGVH2-5 (e.g., V2-5*10);
   the D gene segment is IGHD6-19 (e.g., IGHD6-19*01), IGHD3-10 (e.g., IGHD3-10*01) or IGHD3-9 (e.g., IGHD3-9*01); and/or
   the J gene segment is IGHJ6 (e.g., IGHJ6*02), IGHJ4 (e.g., IGHJ4*02) or IGHJ3 (e.g., IGHJ3*02), or
(ii) comprises framework regions FR1, FR2, FR3 and FR4, wherein
   FR1 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR2 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations,
   FR3 aligns with human germline V gene segment IGHV1-18 (e.g., V1-18*01), IGVH3-20 (e.g. V3-20*d01), IGVH3-11 (e.g, V3-11*01) or IGVH2-5 (e.g., V2-5*10), optionally with 1, 2, 3, 4 or 5 amino acid alterations, and/or
   FR4 aligns with human germline J gene segment IGJH6 (e.g., J_{H}6*02), IGJH4 (e.g., J_{H}4*02) or IGJH3 (e.g., J_{H}3*02), optionally with 1, 2, 3, 4 or 5 amino acid alterations.

Sentence 9. An antibody according to any of the preceding sentences, comprising an antibody VL domain which
(i) is derived from recombination of a human light chain V gene segment and a human light chain J gene segment, wherein
   the V segment is IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), and/or
   the J gene segment is IGKJ4 (e.g., IGKJ4*01), IGKJ2 (e.g., IGKJ2*04), IGLJ3 (e.g., IGKJ3*01) or IGKJ1 (e.g., IGKJ1*01); or
(ii) comprises framework regions FR1, FR2, FR3 and FR4, wherein
   FR1 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR2 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations,
   FR3 aligns with human germline V gene segment IGKV2-28 (e.g., IGKV2-28*01), IGKV3-20 (e.g., IGKV3-20*01), IGKV1D-39 (e.g., IGKV1D-39*01) or IGKV3-11 (e.g., IGKV3-11*01), optionally with 1, 2, 3, 4 or 5 amino acid alterations, and/or
   FR4 aligns with human germline J gene segment IGKJ4 (e.g., IGKJ4*01), IGKJ2 (e.g., IGKJ2*04), IGKJ3 (e.g., IGKJ3*01) or IGKJ1 (e.g., IGKJ1*01), optionally with 1, 2, 3, 4 or 5 amino acid alterations.

Sentence 10. An antibody according to any of the preceding sentences, comprising an antibody VH domain which is the VH domain of STIM001, STIM002, STIM002-B, STIM003, STIM004, STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009.

Sentence 11. An antibody according to any of the preceding sentences, comprising an antibody VL domain which is the VL domain of STIM001, STIM002, STIM002-B, STIM003, STIM004, STIM005, STIM006, STIM007, STIM008 or STIM009, or which has an amino acid sequence at least 90% identical to the antibody VL domain sequence of STIM001, STIM002, STIM002-B, STIM003, STIM004, STIM005, STIM006, STIM007, STIM008 or STIM009.

Sentence 12. An antibody according to sentence 11, comprising
an antibody VH domain which is selected from the VH domain of STIM001, STIM002, STIM002-B, STIM003, STIM004, STIM005, STIM006, STIM007, STIM008 or STIM009, or which has an amino acid sequence at least 90% identical to the antibody VH domain sequence of STIM001, STIM002, STIM002-B, STIM003, STIM004, STIM005, STIM006, STIM007, STIM008 or STIM009, and
an antibody VL domain which is the VL domain of said selected antibody, or which has an amino acid sequence at least 90% identical to the antibody VL domain sequence of said selected antibody.

Sentence 13. An antibody according to sentence 12, comprising the STIM003 VH domain and the STIM003 VL domain.

Sentence 14. An antibody according to any of the preceding sentences, comprising an antibody constant region.

Sentence 15. An antibody according to sentence 14, wherein the constant region comprises a human heavy and/or light chain constant region.

Sentence 16. An antibody according to sentence 14 or sentence 15, wherein the constant region is Fc effector positive.

Sentence 17. An antibody according to sentence 16, comprising an Fc region that has enhanced ADCC, ADCP and/or CDC function compared with a native human Fc region.

Sentence 18. An antibody according to any of sentences 14 to 17, wherein the antibody is an IgG1.

Sentence 19. An antibody according to sentence 17 or sentence 18, wherein the antibody is afucosylated.

Sentence 20. An antibody according to any of the preceding sentences which is conjugated to a cytotoxic drug or pro-drug.

Sentence 21. An antibody according to any of the preceding sentences, which is a multispecific antibody.

Sentence 22. An isolated antibody that competes for binding to human ICOS with a human IgG1 antibody comprising the heavy and light chain complementarity determining regions of STIM001, STIM002, STIM002-B, STIM003, STIM004, STIM005, STIM006, STIM007, STIM008 or STIM009.

Sentence 23. An isolated antibody that binds the extracellular domain of human and mouse ICOS with an affinity (KD) of less than 50 nM as determined by surface plasmon resonance.

Sentence 24. An antibody according to sentence 23, wherein the antibody binds the extracellular domain of human and mouse ICOS with an affinity (KD) of less than 5 nM as determined by surface plasmon resonance.

Sentence 25. An antibody according to sentence 23 or sentence 24, wherein the KD of binding the extracellular domain of human ICOS is within 10-fold of the KD of binding the extracellular domain of mouse ICOS.

Sentence 26. A composition comprising an isolated antibody according to any of the preceding sentences and a pharmaceutically acceptable excipient.

Sentence 27. A composition comprising isolated nucleic acid encoding an antibody according to any of sentences 1 to 25 and a pharmaceutically acceptable excipient.

Sentence 28. A method of depleting regulatory T-cells and/or increasing effector T-cell response in a patient comprising administering a composition according to sentence 26 to the patient.

Sentence 29. A method of treating a disease or condition amenable to therapy by depleting regulatory T-cells and/or increasing effector T-cell response in a patient, the method comprising administering a composition according to sentence 26 to the patient.

Sentence 30. A composition according to sentence 26 for use in a method of treatment of the human body by therapy.

Sentence 31. A composition for use according to sentence 30, for use in depleting regulatory T-cells and/or increasing effector T-cell response in a patient.

Sentence 32. A composition for use according to sentence 30, for use in treating a disease or condition amenable to therapy by depleting regulatory T-cells and/or increasing effector T-cell response in a patient.

Sentence 33. A method according to sentence 29, or a composition for use according to sentence 32, wherein the disease is a cancer or a solid tumour.

Sentence 34. A method or a composition for use according to any of sentences 29 to 33, wherein the method comprises administering the antibody and another therapeutic agent to the patient.

Sentence 35. A method or composition for use according to sentence 34, wherein the therapeutic agent is an anti-PD-L1 antibody.

Sentence 36. A method or composition for use according to sentence 35, wherein the anti-ICOS antibody and the anti-PD-L1 antibody are each able to mediate ADCC, ADCP and/or CDC.

Sentence 37. A method or composition for use according to sentence 35, wherein the anti-ICOS antibody is a human IgG1 antibody and the anti-PD-L1 antibody is a human IgG1 antibody.

Sentence 38. A method or composition for use according to sentence 34, wherein the other therapeutic agent is IL-2.

Sentence 39. A method or composition for use according to any of sentences 34 to 38, wherein the method comprises administering the anti-ICOS antibody after administering the other therapeutic agent.

Sentence 40. A method or a composition for use according to any of sentences 28 to 39, wherein
the anti-ICOS antibody is conjugated to a pro-drug, and wherein
the method or use comprises
administering the anti-ICOS antibody to a patient and
selectively activating the pro-drug at a target tissue site.

Sentence 41. A method or a composition for use according to sentence 40, wherein the patient has a solid tumour and the method comprises selectively activating the pro-drug in the tumour.

Sentence 42. A method or a composition for use according to sentence 40 or sentence 41, comprising selectively activating the pro-drug through photoactivation.

Sentence 43. Combination of anti-ICOS human IgG1 antibody and anti-PD-L1 human IgG1 antibody for use in a method of treating cancer.

Sentence 44. Combination according to sentence 43, wherein the anti-ICOS antibody and the anti-PD-L1 antibody are provided in separate compositions for administration.

Sentence 45. A method or composition for use according to sentence 37, or a combination according to sentence 43 or sentence 44, wherein the human IgG1 constant region has a wild type amino acid sequence shown in the appended sequence listing.

Sentence 46. Anti-ICOS antibody for use in a method of reducing or reversing a surge in ICOS-positive regulatory T-cells in a patient, wherein the surge results from treatment of the patient with another therapeutic agent.

Sentence 47. A method of treating a patient, the method comprising reducing or reversing a surge in ICOS-positive regulatory T-cells in the patient, wherein the surge results from treatment of the patient with another therapeutic agent.

Sentence 48. Anti-ICOS antibody for use in a method of treating a patient, the method comprising administering the anti-ICOS antibody to a patient who has an increased level of ICOS-positive regulatory T-cells following treatment with another therapeutic agent.

Sentence 49. A method of treating a patient, the method comprising administering an anti-ICOS antibody to a patient who has an increased level of ICOS-positive regulatory T-cells following treatment with another therapeutic agent.

Sentence 50. An anti-ICOS antibody for use according to sentence 46 or sentence 48, or a method according to sentence 47 or sentence 49, wherein the method comprises administering a therapeutic agent to the patient, determining that the patient has an increased level of ICOS-positive regulatory T-cells following the treatment with said agent, and administering an anti-ICOS antibody to the patient to reduce the level of regulatory T-cells.

Sentence 51. An anti-ICOS antibody for use or a method according to any of sentences 46 to 50, wherein the therapeutic agent is IL-2 or an immunomodulatory antibody (e.g., anti-PDL-1, anti-PD-1 or anti-CTLA-4).

Sentence 52. An anti-ICOS antibody for use or a method according to any of sentences 46 to 51, wherein the method comprises treating a tumour, e.g., melanoma, such as metastatic melanoma.

Sentence 53. Anti-ICOS antibody for use in a method of treating cancer in a patient by in vivo vaccination of the patient against their cancer cells, the method comprising
treating the patient with a therapy that causes immunological cell death of the cancer cells, resulting in presentation of antigen to antigen-specific effector T-cells, and
administering an anti-ICOS antibody to the patient, wherein the anti-ICOS antibody enhances the antigen-specific effector T-cell response.

Sentence 54. A method of treating cancer in a patient by in vivo vaccination of the patient against their cancer cells, the method comprising
treating the patient with a therapy that causes immunological cell death of the cancer cells, resulting in presentation of antigen to antigen-specific effector T-cells, and
administering an anti-ICOS antibody to the patient, wherein the anti-ICOS antibody enhances the antigen-specific effector T-cell response.

Sentence 55. A method of treating cancer in a patient by in vivo vaccination of the patient against their cancer cells, the method comprising administering an anti-ICOS antibody to the patient, wherein
the patient is one who has been previously treated with a therapy that causes immunological cell death of the cancer cells, resulting in presentation of antigen to antigen-specific effector T-cells, and wherein
the anti-ICOS antibody enhances the antigen-specific effector T-cell response.

Sentence 56. Anti-ICOS antibody for use or a method according to any of sentences 53 to 55, wherein the therapy that causes immunological cell death is radiation of the cancer cells, administration of a chemotherapeutic agent and/or administration of an antibody directed to a tumour-associated antigen.

Sentence 57. Anti-ICOS antibody for use or a method according to sentence 56, wherein the chemotherapeutic agent is oxaliplatin.

Sentence 58. Anti-ICOS antibody for use or a method according to sentence 56, wherein the tumour-associated antigen is HER2 or CD20.

Sentence 59. Anti-ICOS antibody for use in a method of vaccinating a patient, the method comprising administering the antibody and a vaccine composition to the patient.

Sentence 60. A method of vaccinating a patient, the method comprising administering an anti-ICOS antibody and a vaccine composition to the patient.

Sentence 61. Anti-ICOS antibody for use according to sentence 59, or a method according to sentence 60, wherein the vaccine composition is a vaccine against hepatitis B, malaria or HIV.

Sentence 62. Anti-ICOS antibody for use in a method of treating a cancer in a patient, wherein the cancer is or has been characterised as being positive for expression of ICOS ligand and/or FOXP3.

Sentence 63. A method of treating a cancer in a patient, wherein the cancer is or has been characterised as being positive for expression of ICOS ligand and/or FOXP3, the method comprising administering an anti-ICOS antibody to the patient.

Sentence 64. Anti-ICOS antibody for use according to sentence 62, or a method according to sentence 63, wherein the method comprises:
testing a sample from a patient to determine that the cancer expresses ICOS ligand and/or FOXP3;
selecting the patient for treatment with the anti-ICOS antibody; and
administering the anti-ICOS antibody to the patient.

Sentence 65. Anti-ICOS antibody for use according to sentence 62, or a method according to sentence 63, wherein the method comprises administering an anti-ICOS antibody to a patient from whom a test sample has indicated that the cancer is positive for expression of ICOS ligand and/or FOXP3.

Sentence 66. Anti-ICOS antibody for use or a method according to sentence 64 or sentence 65, wherein the sample is biopsy sample of a solid tumour.

Sentence 67. Anti-ICOS antibody for use in a method of treating a cancer in a patient, wherein the cancer is or has been characterised as being refractory to treatment with an immunooncology drug, e.g., anti-CTLA-4 antibody, anti-PD1 antibody, anti-PD-L1 antibody, anti-CD137 antibody or anti-GITR antibody.

Sentence 68. A method of treating a cancer in a patient, wherein the cancer is or has been characterised as being refractory to treatment with an immunooncology drug, e.g., anti-CTLA-4 antibody, anti-PD1 antibody, anti-PD-L1 antibody, anti-CD137 antibody or anti-GITR antibody, the method comprising administering an anti-ICOS antibody to the patient.

Sentence 69. Anti-ICOS antibody for use according to sentence 67 or a method according to sentence 68, wherein the method comprises:
treating the patient with the immunooncology drug;
determining that the cancer is not responsive to the drug;
selecting the patient for treatment with the anti-ICOS antibody; and
administering the anti-ICOS antibody to the patient.

Sentence 70. Anti-ICOS antibody for use according to sentence 67, or a method according to sentence 68, wherein the method comprises administering an anti-ICOS antibody to a patient whose cancer was not responsive to prior treatment with the immunooncology drug.

Sentence 71. Anti-ICOS antibody for use or a method according to any of sentences 62 to 70, wherein the cancer is a tumour derived from cells that have acquired ability to express ICOS ligand.

Sentence 72. Anti-ICOS antibody for use or a method according to sentence 71, wherein the cancer is melanoma.

Sentence 73. Anti-ICOS antibody for use or a method according to any of sentences 62 to 70, wherein the cancer is derived from an antigen-presenting cell, such as a B lymphocyte (e.g., B cell lymphoma, such as diffused large B cell lymphoma) or a T lymphocyte.

Sentence 74. Anti-ICOS antibody for use or a method according to any of sentences 62 to 70, wherein the cancer is resistant to treatment with an anti-CD20 antibody.

Sentence 75. Anti-ICOS antibody for use or a method according to sentence 74, wherein the cancer is B cell lymphoma.

Sentence 76. Anti-ICOS antibody for use or a method according to sentence 75, wherein the anti-CD20 antibody is rituximab.

Sentence 77. Anti-ICOS antibody for use or a method according to any of sentences 74 to 76, wherein the method comprises treating the patient with the anti-CD20 antibody;
determining that the cancer is not responsive to the anti-CD20 antibody;
testing a sample from a patient to determine that the cancer expresses ICOS ligand;
selecting the patient for treatment with the anti-ICOS antibody; and
administering the anti-ICOS antibody to the patient.

Sentence 78. Anti-ICOS antibody for use or a method according to any of sentences 74 to 76, wherein the method comprises administering an anti-ICOS antibody to a patient whose cancer was not responsive to prior treatment with anti-CD20 antibody.

Sentence 79. Anti-ICOS antibody for use or a method according to any of sentences 52 to 78, wherein the cancer is a solid tumour.

Sentence 80. Anti-ICOS antibody for use or a method according to any of sentences 52 to 78, wherein the cancer is a haemotological liquid tumour.

Sentence 81. Anti-ICOS antibody for use or a method according to sentence 79 or 80, wherein the tumour is high in regulatory T-cells.

Sentence 82. Anti-ICOS antibody for use or a method according to any of sentences 43 to 81, wherein the anti-ICOS antibody is as defined in any of sentences 1 to 25 or is provided in a composition according to sentence 26.

Sentence 83. A transgenic non-human mammal having a genome comprising a human or humanised immunoglobulin locus encoding human variable region gene segments, wherein the mammal does not express ICOS.

Sentence 84. A method of producing an antibody that binds the extracellular domain of human and non-human ICOS, comprising
(a) immunising a mammal according to sentence 83 with human ICOS antigen;
(b) isolating antibodies generated by the mammal;
(c) testing the antibodies for ability to bind human ICOS and non-human ICOS; and
(d) selecting one or more antibodies that binds both human and non-human ICOS.

Sentence 85. A method according to sentence 84, comprising immunising the mammal with cells expressing human ICOS.

Sentence 86. A method according to sentence 84 or sentence 85, comprising
(c) testing the antibodies for ability to bind human ICOS and non-human ICOS using surface plasmon resonance and determining binding affinities; and
(d) selecting one or more antibodies for which the KD of binding to human ICOS is less than 50 nM and the KD of binding to non-human ICOS is less than 500 nM.

Sentence 87. A method according to sentence 86, comprising
(d) selecting one or more antibodies for which the KD of binding to human ICOS is less than 10 nM and the KD of binding to non-human ICOS is less than 100 nM.

Sentence 88. A method according to any of sentences 84 to 87, comprising
(c) testing the antibodies for ability to bind human ICOS and non-human ICOS using surface plasmon resonance and determining binding affinities; and
(d) selecting one or more antibodies for which the KD of binding to human ICOS is within 10-fold of the KD of binding to non-human ICOS.

Sentence 89. A method according to sentence 88, comprising
(d) selecting one or more antibodies for which the KD of binding to human ICOS is within 5-fold of the KD of binding to non-human ICOS.

Sentence 90. A method according to any of sentences 84 to 89, comprising testing the antibodies for ability to bind non-human ICOS from the same species as the mammal.

Sentence 91. A method according to any of sentences 84 to 90, comprising testing the antibodies for ability to bind non-human ICOS from a different species as the mammal.

Sentence 92. A method according to any of sentences 84 to 91, wherein the mammal is a mouse or a rat.

Sentence 93. A method according to any of sentences 84 to 92, wherein the non-human ICOS is mouse ICOS or rat ICOS.

Sentence 94. A method according to any of sentences 84 to 93, wherein the human or humanised immunoglobulin locus comprises human variable region gene segments upstream of an endogenous constant region.

Sentence 95. A method according to sentence 94, comprising
(a) immunising a mammal according to sentence 83 with human ICOS antigen, wherein the mammal is a mouse;
(b) isolating antibodies generated by the mouse;
(c) testing the antibodies for ability to bind human ICOS and mouse ICOS; and
(d) selecting one or more antibodies that binds both human and mouse ICOS.

Sentence 96. A method according to any of sentences 84 to 95, comprising isolating nucleic acid encoding an antibody heavy chain variable domain and/or an antibody light chain variable domain.

Sentence 97. A method according to any of sentences 84 to 96, wherein the mammal generates antibodies through recombination of human variable region gene segments and an endogenous constant region.

Sentence 98. A method according to sentence 96 or sentence 97, comprising conjugating the nucleic acid encoding the heavy and/or light chain variable domain to a nucleotide sequence encoding a human heavy chain constant region and/or human light chain constant region respectively.

Sentence 99. A method according to any of sentences 96 to 98, comprising introducing the nucleic acid into a host cell.

Sentence 100.A method according to sentence 99, comprising culturing the host cell under conditions for expression of the antibody, or of the antibody heavy and/or light chain variable domain.

Sentence 101.An antibody, or antibody heavy and/or light chain variable domain, produced by the method according to any of sentences 84 to 100.

Sentence 102.A method of selecting an antibody that binds ICOS, optionally for selecting an ICOS agonist antibody, the assay comprising:
providing an array of antibodies immobilised (attached or adhered) to a substrate in a test well;
adding ICOS-expressing cells (e.g., activated primary T-cells, or MJ cells) to the test well;
observing morphology of the cells;
detecting shape change in the cells from rounded to flattened against the substrate within the well; wherein the shape change indicates that the antibody is an antibody that binds ICOS, optionally an ICOS agonist antibody;
selecting the antibody from the test well;
   expressing nucleic acid encoding the CDRs of the selected antibody; and
   formulating the antibody into a composition comprising one or more additional components. Alternative sentences describing anti-ICOS antibodies are described below:

Sentence 1a. An antibody or a fragment thereof which specifically binds to human ICOS (hICOS) (SEQ ID NO: 508, 507 and/or 506), and:
a) competes for binding to said hICOS with the antibody STIM001, wherein the antibody or fragment comprises a VH domain which comprises the CDRH3 sequence of SEQ ID NO: 365, or the CDRH3 sequence of SEQ ID NO:365 comprising 3, 2 or 1 amino acid substitution(s);
b) competes for binding to said hICOS with the antibody STIM002, wherein the antibody or fragment comprises a VH domain which comprises the CDRH3 sequence of SEQ ID NO: 379, or the CDRH3 sequence of SEQ ID NO:379 comprising 3, 2 or 1 amino acid substitution(s);
c) competes for binding to said hICOS with the antibody STIM002-B, wherein the antibody or fragment comprises a VH domain which comprises the CDRH3 sequence of SEQ ID NO:393, or the CDRH3 sequence of SEQ ID NO:393 comprising 3, 2 or 1 amino acid substitution(s);
d) competes for binding to said hICOS with the antibody STIM003, wherein the antibody or fragment comprises a VH domain which comprises the CDRH3 sequence of SEQ ID NO: 407, or the CDRH3 sequence of SEQ ID NO:407 comprising 3, 2 or 1 amino acid substitution(s);
e) competes for binding to said hICOS with the antibody STIM004, wherein the antibody or fragment comprises a VH domain which comprises the CDRH3 sequence of SEQ ID NO: 421, or the CDRH3 sequence of SEQ ID NO:421 comprising 3, 2 or 1 amino acid substitution(s);
f) competes for binding to said hICOS with the antibody STIM005, wherein the antibody or fragment comprises a VH domain which comprises the CDRH3 sequence of SEQ ID NO: 437, or the CDRH3 sequence of SEQ ID NO:437 comprising 3, 2 or 1 amino acid substitution(s);
g) competes for binding to said hICOS with the antibody STIM006, wherein the antibody or fragment comprises a VH domain which comprises the CDRH3 sequence of SEQ ID NO: 451, or the CDRH3 sequence of SEQ ID NO:451 comprising 3, 2 or 1 amino acid substitution(s);
h) competes for binding to said hICOS with the antibody STIM007, wherein the antibody or fragment comprises a VH domain which comprises the CDRH3 sequence of SEQ ID NO: 465, or the CDRH3 sequence of SEQ ID NO:465 comprising 3, 2 or 1 amino acid substitution(s);
i) competes for binding to said hICOS with the antibody STIM008, wherein the antibody or fragment comprises a VH domain which comprises the CDRH3 sequence of SEQ ID NO: 479, or the CDRH3 sequence of SEQ ID NO:479 comprising 3, 2 or 1 amino acid substitution(s); or
j) competes for binding to said hICOS with the antibody STIM009, wherein the antibody or fragment comprises a VH domain which comprises the CDRH3 sequence of SEQ ID NO: 493, or the CDRH3 sequence of SEQ ID NO:493 comprising 3, 2 or 1 amino acid substitution(s).

Sentence 2a. The antibody or a fragment thereof according to sentence 1a, wherein the VH domain comprises the CDRH1 sequence of:
a) SEQ ID NO:363, or the CDRH1 sequence of SEQ ID NO:363 comprising 1 amino acid substitution;
b) SEQ ID NO:377, or the CDRH1 sequence of SEQ ID NO:377 comprising 1 amino acid substitution;
c) SEQ ID NO:391, or the CDRH1 sequence of SEQ ID NO:391 comprising 1 amino acid substitution;
d) SEQ ID NO:405, or the CDRH1 sequence of SEQ ID NO:405 comprising 1 amino acid substitution;
e) SEQ ID NO:419, or the CDRH1 sequence of SEQ ID NO:419 comprising 1 amino acid substitution;
f) SEQ ID NO:435, or the CDRH1 sequence of SEQ ID NO:435 comprising 1 amino acid substitution;
g) SEQ ID NO:449, or the CDRH1 sequence of SEQ ID NO:449 comprising 1 amino acid substitution;
h) SEQ ID NO:463, or the CDRH1 sequence of SEQ ID NO:463 comprising 1 amino acid substitution; or
i) SEQ ID NO:477, or the CDRH1 sequence of SEQ ID NO:477 comprising 1 amino acid substitution.

j) SEQ ID NO:491, or the CDRH1 sequence of SEQ ID NO:491 comprising 1 amino acid substitution.

Sentence 3a. The antibody or a fragment thereof according to sentence 1a or sentence 2a, wherein the VH domain comprises the CDRH2 sequence of:
a) SEQ ID NO:364, or the CDRH2 sequence of SEQ ID NO:364 comprising 2 or 1 amino acid substitution(s);
b) SEQ ID NO:378, or the CDRH2 sequence of SEQ ID NO:378 comprising 2 or 1 amino acid substitution(s);
c) SEQ ID NO:392, or the CDRH2 sequence of SEQ ID NO:392 comprising 2 or 1 amino acid substitution(s);
d) SEQ ID NO:406, or the CDRH2 sequence of SEQ ID NO:406 comprising 2 or 1 amino acid substitution(s);
e) SEQ ID NO:420, or the CDRH2 sequence of SEQ ID NO:420 comprising 2 or 1 amino acid substitution(s);
f) SEQ ID NO:436, or the CDRH2 sequence of SEQ ID NO:436 comprising 2 or 1 amino acid substitution(s);
g) SEQ ID NO:450, or the CDRH2 sequence of SEQ ID NO:450 comprising 2 or 1 amino acid substitution(s);
h) SEQ ID NO:464, or the CDRH2 sequence of SEQ ID NO:464 comprising 2 or 1 amino acid substitution(s);
i) SEQ ID NO:478, or the CDRH2 sequence of SEQ ID NO:478 comprising 2 or 1 amino acid substitution(s); or
j) SEQ ID NO:492, or the CDRH2 sequence of SEQ ID NO:492 comprising 2 or 1 amino acid substitution(s).

Sentence 4a. The antibody or a fragment thereof according to any preceding sentence, wherein the VH domain comprises:
a) an amino acid sequence of SEQ ID NO:366, or a heavy chain variable domain amino acid sequence that is at least 98% identical to SEQ ID NO:366;
b) an amino acid sequence of SEQ ID NO:380, or a heavy chain variable domain amino acid sequence that is at least 98% identical to SEQ ID NO:380;
c) an amino acid sequence of SEQ ID NO:394, or a heavy chain variable domain amino acid sequence that is at least 98% identical to SEQ ID NO:394;
d) an amino acid sequence of SEQ ID NO:408, or a heavy chain variable domain amino acid sequence that is at least 98% identical to SEQ ID NO:408;
e) an amino acid sequence of SEQ ID NO:422, or a heavy chain variable domain amino acid sequence that is at least 98% identical to SEQ ID NO:422;
f) an amino acid sequence of SEQ ID NO:438, or a heavy chain variable domain amino acid sequence that is at least 98% identical to SEQ ID NO:438;
g) an amino acid sequence of SEQ ID NO:452, or a heavy chain variable domain amino acid sequence that is at least 98% identical to SEQ ID NO:452;
h) an amino acid sequence of SEQ ID NO:466, or a heavy chain variable domain amino acid sequence that is at least 98% identical to SEQ ID NO:466;
i) an amino acid sequence of SEQ ID NO:480, or a heavy chain variable domain amino acid sequence that is at least 98% identical to SEQ ID NO:480; or
j) an amino acid sequence of SEQ ID NO:494, or a heavy chain variable domain amino acid sequence that is at least 98% identical to SEQ ID NO:494.

Sentence 5a. The antibody or fragment according to any preceding sentence comprising first and second copies of said VH domain.

Sentence 6a. The antibody or a fragment thereof according to any preceding sentence comprising a VL domain, which comprises the CDRL1 sequence of:
a) SEQ ID NO:370, or the CDRL1 sequence of SEQ ID NO:370 comprising one amino acid substitution;
b) SEQ ID NO:384, or the CDRL1 sequence of SEQ ID NO:384 comprising one amino acid substitution;
c) SEQ ID NO:398, or the CDRL1 sequence of SEQ ID NO:398 comprising one amino acid substitution;
d) SEQ ID NO:412, or the CDRL1 sequence of SEQ ID NO:412 comprising one amino acid substitution;
e) SEQ ID NO:426, or the CDRL1 sequence of SEQ ID NO:426 comprising one amino acid substitution;
f) SEQ ID NO:442, or the CDRL1 sequence of SEQ ID NO:442 comprising one amino acid substitution;
g) SEQ ID NO:456, or the CDRL1 sequence of SEQ ID NO:456 comprising one amino acid substitution;
h) SEQ ID NO:470, or the CDRL1 sequence of SEQ ID NO:470 comprising one amino acid substitution; or
i) SEQ ID NO:484, or the CDRL1 sequence of SEQ ID NO:484 comprising one amino acid substitution.

j) SEQ ID NO:498, or the CDRL1 sequence of SEQ ID NO:498 comprising one amino acid substitution.

Sentence 7a. The antibody or a fragment thereof according to any preceding sentence comprising a or said VL domain, which VL domain comprises the CDRL2 sequence of:
a) SEQ ID NO:371, or the CDRL2 sequence of SEQ ID NO:371 comprising 1 amino acid substitution;
b) SEQ ID NO:385, or the CDRL2 sequence of SEQ ID NO:385 comprising 1 amino acid substitution;
c) SEQ ID NO:399, or the CDRL2 sequence of SEQ ID NO:399 comprising 1 amino acid substitution;
d) SEQ ID NO:413, or the CDRL2 sequence of SEQ ID NO:413 comprising 1 amino acid substitution;
e) SEQ ID NO:427, or the CDRL2 sequence of SEQ ID NO:427 comprising 1 amino acid substitution;
f) SEQ ID NO:443, or the CDRL2 sequence of SEQ ID NO:443 comprising 1 amino acid substitution;
g) SEQ ID NO:457, or the CDRL2 sequence of SEQ ID NO:457 comprising 1 amino acid substitution;
h) SEQ ID NO:471, or the CDRL2 sequence of SEQ ID NO:471 comprising 1 amino acid substitution;
i) SEQ ID NO:485, or the CDRL2 sequence of SEQ ID NO:485 comprising 1 amino acid substitution; or
j) SEQ ID NO:499, or the CDRL2 sequence of SEQ ID NO:499 comprising 1 amino acid substitution.

Sentence 8a. The antibody or a fragment thereof according to any preceding sentence comprising a or said VL domain, which V_{L} domain comprises the CDRL3 sequence of:
a) SEQ ID NO:372, or the CDRL3 sequence of SEQ ID NO:372 comprising 2 or 1 amino acid substitution(s);
b) SEQ ID NO:386, or the CDRL3 sequence of SEQ ID NO:386 comprising 2 or 1 amino acid substitution(s);
c) SEQ ID NO:400, or the CDRL3 sequence of SEQ ID NO:400 comprising 2 or 1 amino acid substitution(s);
d) SEQ ID NO:414, or the CDRL3 sequence of SEQ ID NO:414 comprising 2 or 1 amino acid substitution(s);
e) SEQ ID NO:428, or the CDRL3 sequence of SEQ ID NO:428 comprising 2 or 1 amino acid substitution(s);
f) SEQ ID NO:444, or the CDRL3 sequence of SEQ ID NO:444 comprising 2 or 1 amino acid substitution(s);
g) SEQ ID NO:458, or the CDRL3 sequence of SEQ ID NO:458 comprising 2 or 1 amino acid substitution(s);
h) SEQ ID NO:472, or the CDRL3 sequence of SEQ ID NO:472 comprising 2 or 1 amino acid substitution(s);
i) SEQ ID NO:486, or the CDRL3 sequence of SEQ ID NO:486 comprising 2 or 1 amino acid substitution(s); or
j) SEQ ID NO:500, or the CDRL3 sequence of SEQ ID NO:500 comprising 2 or 1 amino acid substitution(s).

Sentence 9a. The antibody or a fragment thereof according to any preceding sentence, comprising a or said VL domain, wherein the VL domain comprises an amino acid sequence of:
a) SEQ ID NO:373, or a light chain variable domain amino acid sequence that is at least 98% identical to SEQ ID NO:373;
b) SEQ ID NO:387, or a light chain variable domain amino acid sequence that is at least 98% identical to SEQ ID NO:387;
c) SEQ ID NO:401, or a light chain variable domain amino acid sequence that is at least 98% identical to SEQ ID NO:401;
d) SEQ ID NO:415, or a light chain variable domain amino acid sequence that is at least 98% identical to SEQ ID NO:415;
e) SEQ ID NO:429, or a light chain variable domain amino acid sequence that is at least 98% identical to SEQ ID NO:429;
f) SEQ ID NO:445, or a light chain variable domain amino acid sequence that is at least 98% identical to SEQ ID NO:445;
g) SEQ ID NO:459, or a light chain variable domain amino acid sequence that is at least 98% identical to SEQ ID NO:459;
h) SEQ ID NO:473, or a light chain variable domain amino acid sequence that is at least 98% identical to SEQ ID NO:473;
i) SEQ ID NO:487, or a light chain variable domain amino acid sequence that is at least 98% identical to SEQ ID NO:487; or
j) SEQ ID NO:501, or a light chain variable domain amino acid sequence that is at least 98% identical to SEQ ID NO:501.

Sentence 10a. The antibody or fragment according to any one of sentences 6a to 9a, comprising first and second copies of the a or said VL domain.

Sentence 11. The antibody or fragment according to any preceding sentence, wherein the amino acid substitutions are conservative amino acid substitutions, optionally wherein the conservative substitutions are from one of six groups (each group containing amino acids that are conservative substitutions for one another) selected from:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Sentence 12a. An antibody or fragment thereof which specifically binds to an epitope that is:

a) Identical to an epitope to which the antibody STIM001 specifically binds;
b) Identical to an epitope to which the antibody STIM002 specifically binds;
c) Identical to an epitope to which the antibody STIM002-B specifically binds;
d) Identical to an epitope to which the antibody STIM003 specifically binds;
e) Identical to an epitope to which the antibody STIM004 specifically binds;
f) Identical to an epitope to which the antibody STIM005 specifically binds;
g) Identical to an epitope to which the antibody STIM006 specifically binds;
h) Identical to an epitope to which the antibody STIM007 specifically binds;
i) Identical to an epitope to which the antibody STIM008 specifically binds; or
j) Identical to an epitope to which the antibody STIM009 specifically binds.

Sentence 13a. The antibody or fragment according to sentence 12a, wherein the epitope is identified by unrelated amino acid scanning, or by X-ray crystallography.

Sentence 14a. The antibody or fragment according to sentence 13a, wherein the contact residues of the epitope are defined by a reduction in affinity of at least 10-fold in an unrelated amino acid scan, e.g. an alanine scan as determined by SPR.

Sentence 15a. An antibody or fragment thereof which:
a) Competes for binding to hICOS with the antibody STIM001;
b) Competes for binding to hICOS with the antibody STIM002;
c) Competes for binding to hICOS with the antibody STIM002-B;
d) Competes for binding to hICOS with the antibody STIM003;
e) Competes for binding to hICOS with the antibody STIM004;
f) Competes for binding to hICOS with the antibody STIM005;
g) Competes for binding to hICOS with the antibody STIM006;
h) Competes for binding to hICOS with the antibody STIM007;
i) Competes for binding to hICOS with the antibody STIM008; or
j) Competes for binding to hICOS with the antibody STIM009.

Sentence 16a. The antibody or fragment according to any preceding sentence which specifically binds to cynomolgus ICOS (Seq ID No:513, Seq ID NO: 513 or Seq ID No: 514) and/or mouse ICOS (Seq ID No:510, Seq ID No:511 or Seq ID No:512).

Sentence 17a. The antibody or fragment according to any preceding sentence which specifically binds to a hICOS isoform or natural variant, a mouse ICOS isoform or natural variant and/or a cynomolgus ICOS isoform or natural variant.

Sentence 18a. The antibody or fragment according to sentence 17a, wherein the hICOS isoform comprises an amino acid sequence as defined by Seq ID no:509.

Sentence 19a. The antibody or fragment according to any preceding sentence, wherein the antibody or fragment comprises a constant region, such as a human constant region, for example an effector-null human constant region, e.g. an IgG4 constant region or an IgG1 constant region, optionally wherein the constant region is IgG4-PE (Seq ID No:199), or a disabled IgG1 (Seq ID No:205).

Sentence 20a. The antibody or fragment according to sentence 19a, wherein the constant region is a murine constant region.

Sentence 21a. The antibody or fragment according to sentence 19a or sentence 20a, wherein the constant region has CDC and/or ADCC activity.

### Configurations and Arrangements Relating to Anti-ICOS Bispecific Antibodies

In a first configuration, there is provided a multispecific antibody (e.g. bispecific antibody or a dual-binding antibody) which binds (and optionally has specificity for) ICOS (e.g. human ICOS) and another target antigen.

In a second configuration, there is provided a composition comprising a multispecific, bispecific or dual-binding antibody as described herein and a pharmaceutically acceptable excipient, diluent or carrier.

In a third configuration, there is provided a multispecific, bispecific or dual-binding antibody as described herein for use in treating or preventing a disease or condition, selected from neurological disease, neoplastic or non-neoplastic disease, chronic viral infections, and malignant tumours; such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

In a fourth configuration, there is provided a use of a multispecific, bispecific or dual-binding antibody as described herein in the manufacture of a medicament for administration to a human for treating or preventing a disease or condition in the human selected from neurological disease, neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

In a fifth configuration, there is provided a method of treating or preventing a disease or condition selected from neurological disease, neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas) in a human, comprising administering to said human a therapeutically effective amount of a multispecific, bispecific or dual-binding antibody as described herein, wherein the disease or condition is thereby treated or prevented.

In a sixth configuration, there is provided a nucleic acid that encodes a heavy chain and/or a light chain of a multispecific antibody as described herein.

In a seventh configuration, there is provided a vector comprising the nucleic acid that encodes a heavy chain and/or a light chain of a multispecific antibody as described herein.

As previously described, the PD-L1 antibodies as provided herein, may be formatted as a multispecific (e.g. bispecific) antibody, as disclosed hereinabove in concepts 37 to 40. In one embodiment disclosed therein, the PD-L1 antibodies as disclosed herein may be formatted in a bispecific antibody which has specificity for both PD-L1 (e.g. human PD-L1) and for ICOS (e.g. an agonist to ICOS, such as human ICOS).

Thus, there is provided a multispecific (e.g. bispecific antibody or a dual-binding antibody) which has specificity for PD-L1 (e.g. human PD-L1) and ICOS (e.g. human ICOS). In one embodiment the multispecific (e.g. bispecific or dual-binding) antibody has agonistic activity against ICOS (e.g. human ICOS).

Various ICOS-containing multispecific antibodies are described in the arrangements below:

Arrangement 1. A multispecific antibody (e.g. bispecific antibody or a dual-binding antibody) which binds (and optionally has specificity for) ICOS (e.g. human ICOS) and another target antigen.

In one embodiment, there is provided a bispecific antibody or a dual-binding antibody which binds ICOS (e.g. human ICOS) and another target antigen. In one embodiment, there is provided a bispecific antibody or a dual-binding antibody which has specificity for ICOS (e.g. human ICOS) and another target antigen. In one embodiment, there is provided a bispecific antibody antibody which binds ICOS (e.g. human ICOS) and another target antigen, and wherein the bispecific antibody format is a mAb². In one embodiment, there is provided a bispecific antibody antibody which binds ICOS (e.g. human ICOS) and another target antigen, and wherein the bispecific antibody format is a mAb², and the binding to another target antigen is provided by a modified constant region (i.e. an Fcab). In one embodiment, there is provided a bispecific antibody antibody which binds ICOS (e.g. human ICOS) and another target antigen which is PD-L1 (e.g. human PD-L1), and wherein the bispecific antibody format is a mAb², and the binding to ICOS is provided by a modified constant region (i.e. an Fcab). In one embodiment, there is provided a bispecific antibody antibody which binds ICOS (e.g. human ICOS) and another target antigen which is PD-L1 (e.g. human PD-L1), and wherein the bispecific antibody format is a mAb², and the binding to ICOS is provided by a modified constant region (i.e. an Fcab) and the binding to PD-L1 is provided by any of the antibodies described in concepts 1 to 70, or by any of the PD-L1 antibodies described in arrangement 5 or 5a below. In one embodiment, there is provided a bispecific antibody antibody which binds ICOS (e.g. human ICOS) and another target antigen which is PD-L1 (e.g. human PD-L1), and wherein the bispecific antibody format is a mAb², and the binding to PD-L1 is provided by a modified constant region (i.e. an Fcab). In one embodiment, there is provided a bispecific antibody antibody which binds ICOS (e.g. human ICOS) and another target antigen which is PD-L1 (e.g. human PD-L1), and wherein the bispecific antibody format is a mAb², and the binding to PD-L1 is provided by a modified constant region (i.e. an Fcab) and the binding to ICOS is provided by any of the antibodies described in sentences 1 to 102 or sentences 1a to 21a.

In one embodiment, the multispecific (e.g. bispecific or dual-binding) antibody has agonistic activity against ICOS (e.g. human ICOS). The another target antigen may be any of the target antigens specified in concept 39. In one embodiment, the another target antigen is an immune checkpoint inhibitor, such as PD-1, PD-L1, CTLA-4, TIGIT, TIM-3, LAG-3 and VISTA, e.g. PD-L1, TIGIT, CTLA-4, TIM-3 and LAG-3. In one embodiment, the another target antigen is an immune modulator, such as BTLA, hHVEM, CSF1R, CCR4, CD39, CD40, CD73, CD96, CXCR2, CXCR4, CD200, GARP, SIRPα, CXCL9, CXCL10, CXCL11 and CD155, e.g. GARP, SIRPα, CXCR4, BTLA, hVEM and CSF1R. In one embodiment, the another target antigen is an immune activator, such as CD137, GITR, OX40, CD40, CXCR3 (e.g. agonistic anti-CXCR3 antibodies), CD27 and CD3, or CD137, GITR, OX40, CD40, CXCR3 (e.g. agonistic anti-CXCR3 antibodies) and CD3, for example CD137, GITR and OX40). In one embodiment, the another target antigen is PD-L1. In one embodiment, the another target antigen is CTLA-4. In one embodiment, the another target antigen is TIGIT. In one embodiment, the another target antigen is TIM-3. In one embodiment, the another target antigen is LAG-3. In one embodiment, the another target antigen is GITR. In one embodiment, the another target antigen is VISTA. In one embodiment, the another target antigen is CD137. In one embodiment, the another target antigen is SIRPα. In one embodiment, the another target antigen is CXCL10. In one embodiment, the another target antigen is CD155. In one embodiment, the another target antigen is CD40. The antibodies against these another target antigens may be any of those described in aspect 1a.

The format of the multispecific, bispecific or dual-binding antibody may be any of the formats disclosed herein, for example as set out in concepts 37 to 40. In particular, the binding and/or specificity for ICOS may be provided by a non-immunoglobulin format, for example, a T-cell receptor binding domain; an immunoglobulin superfamily domain; an agnathan variable lymphocyte receptor; a fibronectin domain (e.g., an Adnectin™); an antibody constant domain (e.g., a CH3 domain, e.g., a CH2 and/or CH3 of an Fcab™) wherein the constant domain is not a functional CH1 domain; an scFv; an (scFv) 2; an sc-diabody; an scFab; a centyrin and an epitope binding domain derived from a scaffold selected from CTLA-4 (Evibody™); a lipocalin domain; Protein A such as Z-domain of Protein A (e.g., an Affibody™ or SpA); an A-domain (e.g., an Avimer™ or Maxibody™); a heat shock protein (such as and epitope binding domain derived from GroEI and GroES); a transferrin domain (e.g., a trans-body); ankyrin repeat protein (e.g., a DARPin™); peptide aptamer; C-type lectin domain (e.g., Tetranectin™); human γ-crystallin or human ubiquitin (an affilin); a PDZ domain; scorpion toxin; and a kunitz type domain of a human protease inhibitor. The binding and/or specificity for another target antigen may be provided by an immunoglobulin-derived antigen-binding protein.

“Specifically binds” has the meaning provided hereinabove. Binding constants, e.g. KD may be determined as described elsewhere herein, and particular KDs of interest are described in arrangement 2 below, and in concept 1 hereinabove (although specified for PD-L1 binding, the values of KD may be equally applied to anti-ICOS binding).

Arrangement 2. A multispecific antibody according to arrangement 1, wherein the ICOS is human ICOS.

Sequences of human ICOS are provided in Seq ID Nos: 506, 507 and 508. In one embodiment, the multispecific antibody is specific for wild type human ICOS. In another embodiment, the multispecific antibody is cross-reactive to an isoform or natural variant of hICOS, for example the isoform of Seq ID No:509. Other isoforms and natural variants are well known to those skilled in the art. In another embodiment, the multispecific antibody is specific for the isoform or natural variant (e.g. the ICOS isoform having the amino acid sequence of Seq ID No:509) over wild type hICOS.

One way to quantify the extent of species cross-reactivity of an antibody, e.g. a multispecific, bispecific or dual-binding antibody is as the fold-difference in its affinity for antigen compared with a different antigen (e.g. fold difference in affinity for human ICOS vs mouse ICOS or fold difference in affinity for wild-type hICOS vs an isoform of hICOS). Affinity may be quantified as KD, referring to the equilibrium dissociation constant of the antibody-antigen reaction as determined by SPR (optionally with the antibody in Fab format as described elsewhere herein). A species or isoform cross-reactive anti-ICOS antibody may have a fold-difference in affinity for binding human and mouse ICOS that is 30-fold or less, 25-fold or less, 20-fold or less, 15-fold or less, 10-fold or less or 5-fold or less. To put it another way, the KD of binding the extracellular domain of hICOS may be within 30-fold, 25-fold, 20-fold, 15-fold, 10-fold or 5-fold of the KD of binding the extracellular domain of mouse ICOS. Antibodies can also be considered cross-reactive if the KD for binding antigen of both species meets a threshold value, e.g., if the KD of binding hICOS and the KD of binding mouse ICOS are both 10 mM or less, preferably 5 mM or less, more preferably 1 mM or less. The KD may be 10 nM or less, 5 nM or less, 2 nM or less, or 1 nM or less. The KD may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less.

An alternative measure of cross-reactivity for binding hICOS and mouse ICOS, or WT hICOS and an isoform of hICOS is the ability of an antibody to neutralise ICOS ligand binding to ICOS receptor, such as in an HTRF assay (as described elsewhere herein). Examples of species cross-reactive antibodies are provided herein, including STIM001, STIM002, STIM002-B, STIM003, STIM005 and STIM006, each of which was confirmed as neutralising binding of human B7-H2 (ICOS ligand) to hICOS and neutralising binding of mouse B7-H2 to mouse ICOS in an HTRF assay. Any of these antibodies or their variants may be selected when an antibody cross-reactive for human and mouse ICOS is desired. A species cross-reactive anti-ICOS antibody may have an IC50 for inhibiting binding of hICOS to human ICOS receptor that is within 25-fold, 20-fold, 15-fold, 10-fold or 5-fold of the IC50 for inhibiting mouse ICOS to mouse ICOS receptor as determined in an HTRF assay. Antibodies can also be considered cross-reactive if the IC50 for inhibiting binding of hICOS to human ICOS receptor and the IC50 for inhibiting binding of mouse ICOS to mouse ICOS receptor are both 1 mM or less, preferably 0.5 mM or less, e.g., 30 nM or less, 20 nM or less, 10 nM or less. The IC50s may be 5 nM or less, 4 nM or less, 3 nM or less or 2 nM or less. In some cases, the IC50s will be at least 0.1 nM, at least 0.5 nM or at least 1 nM.

Affinities may also be as disclosed in concept 27 hereinabove.

Arrangement 3. A multispecific antibody according to arrangement 2, which comprises a VH domain comprising a CDRH1, a CDRH2 and a CDRH3 which VH domain binds (and optionally has specificity for) hICOS.

In one embodiment, the multispecific antibody comprises at least one VH domain which binds to hICOS. For example, the multispecific antibody may comprise a single-chain Fv (scFv), single-chain antibody, a single domain antibody or a domain antibody compositing only the VH¬ region which binds to (and optionally has specificity for) hICOS.

Arrangement 4. A multispecific antibody according to arrangement 2 or arrangement 3, which comprises a VL domain comprising a CDRL1, a CDRL2 and a CDRL3, which VL domain binds (and optionally has specificity for) hICOS.

In one embodiment, the multispecific antibody comprises at least one VL domain which binds to hICOS. For example, the multispecific antibody may comprise a single-chain Fv (scFv), single-chain antibody, a single domain antibody or a domain antibody compositing only the VL region which binds to (and optionally has specificity for) hICOS.

In another embodiment, the multispecific antibody comprises a paired VH and VL domain, including, but not limited to, an intact or full-length antibody, a Fab fragment, a Fab′ fragment, a F(ab′)2 fragment or a Fv fragment.

Arrangement 5. A multispecific antibody according to arrangement 3 or 4, wherein the VH and/or VL domain is any of VH and/or VL domains:
a. of the antibody 7F12, 37A10, 35A9, 36E10, 16G10, 37A10S713, 37A10S714, 37A10S715, 37A10S716, 37A10S717, 37A10S718, 16G10S71, 16G10S72, 16G10S73, 16G10S83, 35A9S79, 35A9S710, 35A9S89 or any other antibody described in WO2016/154177 and US2016/0304610;
b. of the antibody 422.2, H2L5, or any other antibody described in WO2016/120789 and US2016/0215059;
c. of the antibody 314-8, the antibody produced from hybridoma CNCM I-4180, or any other antibody described in WO2014/033327 and US2015/0239978;
d. of the antibody Icos145-1, the antibody produced by hybridoma CNCM I-4179, or any other antibody described in WO2012/131004, U.S. Pat. No. 9,376,493 and US2016/0264666;
e. of the antibody JMAb 136, “136”, or any other antibody described in WO2010/056804;
f. of the antibody MIC-944, 9F3 or any other antibody described in WO99/15553, U.S. Pat. Nos. 7,259,247, 7,132,099, 7,125,551, 7,306,800, 7,722,872, WO05/103086, U.S. Pat. Nos. 8,318,905 and 8,916,155;
g. of any JMAb antibody, e.g., any of JMAb-124, JMAb-126, JMAb-127, JMAb-128, JMAb-135, JMAb-136, JMAb-137, JMAb-138, JMAb-139, JMAb-140, JMAb-141, e.g., JMAb136, or any other antibodye described in WO98/3821, U.S. Pat. No. 7,932,358B2, US2002/156242, U.S. Pat. Nos. 7,030,225, 7,045,615, 7,279,560, 7,226,909, 7,196,175, 7,932,358, 8,389,690, WO02/070010, U.S. Pat. Nos. 7,438,905, 7,438,905, WO01/87981, U.S. Pat. Nos. 6,803,039, 7,166,283, 7,988,965, WO01/15732, U.S. Pat. Nos. 7,465,445 and 7,998,478;
h. of the antibody 17G9 or any other antibody described in WO2014/08911;
i. of any antibody described in WO2012/174338;
j. of any antibody described in US2016/0145344;
k. of any antibody described in WO2011/020024, US2016/002336, US2016/024211 and U.S. Pat. No. 8,840,889; or
l. of any antibody described in U.S. Pat. No. 8,497,244;
m. of the antibody known as GSK3359609;
n. of the antibody known as JTX-2011; or
o. of antibody clone ISA-3 (eBioscience), clone SP98 (Novus Biologicals), clone 1 G1, clone 3G4 (Abnova Corporation), clone 669222 (R&D Systems), clone TQ09 (Creative Diagnostics), or clone C398.4A (BioLegend).

Arrangement 5a. A multispecific antibody according to any preceding arrangement, which comprises the CDRH1, CDRH2, CDR3, CDRL1, CDRL2 and CDRL3, or the VH, or the VL or the VH and VL region:
a. of the antibody 7F12, 37A10, 35A9, 36E10, 16G10, 37A10S713, 37A10S714, 37A10S715, 37A10S716, 37A10S717, 37A10S718, 16G10S71, 16G10S72, 16G10S73, 16G10S83, 35A9S79, 35A9S710, 35A9S89 or any other antibody described in WO2016/154177 and US2016/0304610;
b. of the antibody 422.2, H2L5, or any other antibody described in WO2016/120789 and US2016/0215059;
c. of the antibody 314-8, the antibody produced from hybridoma CNCM I-4180, or any other antibody described in WO2014/033327 and US2015/0239978;
d. of the antibody Icos145-1, the antibody produced by hybridoma CNCM I-4179, or any other antibody described in WO2012/131004, U.S. Pat. No. 9,376,493 and US2016/0264666;
e. of the antibody JMAb 136, “136”, or any other antibody described in WO2010/056804;
f. of the antibody MIC-944, 9F3 or any other antibody described in WO99/15553, U.S. Pat. Nos. 7,259,247, 7,132,099, 7,125,551, 7,306,800, 7,722,872, WO05/103086, U.S. Pat. Nos. 8,318,905 and 8,916,155;
g. of any JMAb antibody, e.g., any of JMAb-124, JMAb-126, JMAb-127, JMAb-128, JMAb-135, JMAb-136, JMAb-137, JMAb-138, JMAb-139, JMAb-140, JMAb-141, e.g., JMAb136, or any other antibodye described in WO98/3821, U.S. Pat. No. 7,932,358B2, US2002/156242, U.S. Pat. Nos. 7,030,225, 7,045,615, 7,279,560, 7,226,909, 7,196,175, 7,932,358, 8,389,690, WO02/070010, U.S. Pat. Nos. 7,438,905, 7,438,905, WO01/87981, U.S. Pat. Nos. 6,803,039, 7,166,283, 7,988,965, WO01/15732, U.S. Pat. Nos. 7,465,445 and 7,998,478;
h. of the antibody 17G9 or any other antibody described in WO2014/08911;
i. of any antibody described in WO2012/174338;
j. of any antibody described in US2016/0145344;
k. of any antibody described in WO2011/020024, US2016/002336, US2016/024211 and U.S. Pat. No. 8,840,889; or
l. of any antibody described in U.S. Pat. No. 8,497,244;
m. of the antibody known as GSK3359609;
n. of the antibody known as JTX-2011; or
o. of antibody clone ISA-3 (eBioscience), clone SP98 (Novus Biologicals), clone 1 G1, clone 3G4 (Abnova Corporation), clone 669222 (R&D Systems), clone TQ09 (Creative Diagnostics), or clone C398.4A (BioLegend).

Arrangement 6. A multispecific antibody according to arrangement 3 or 4, wherein the VH and/or VL domain is any of VH and/or VL domains defined in sentences 1 to 102 or sentences 1a to 21a.

In one embodiment, the anti-ICOS VH and/or VL is as described in GB patent application 1620414.1 (filed 1 Dec. 2016), the contents of which are incorporated herein by reference.

Arrangement 7. A multispecific antibody according to any preceding arrangement, which has agonistic activity against ICOS.

Agonism can be tested for in an in vitro T-cell activation assays, using antibody in soluble form (e.g. in immunoglobulin format or other antibody format comprising two spatially separated antigen-binding sites, e.g., two VH-VL pairs), either including or excluding a cross-linking agent, or using antibody (e.g. multispecific antibody) bound to a solid surface to provide a tethered array of antigen-binding sites. Agonism assays may use a hICOS positive T-lymphocyte cell line such as MJ cells (ATCC CRL-8294) as the target T-cell for activation in such assays. One or more measures of T-cell activation can be determined for a test antibody and compared with a reference molecule or a negative control to determine whether there is a statistically significant (p<0.05) difference in T-cell activation effected by the test antibody (e.g. multispecific antibody) compared with the reference molecule or the control. One suitable measure of T-cell activation is production of cytokines, e.g., IFNγ, TNFα or IL-2. A skilled person will include suitable controls as appropriate, standardising assay conditions between test antibody and control. A suitable negative control is an antibody in the same format (e.g., isotype control) that does not bind ICOS, e.g., an antibody (e.g. multispecific antibody) specific for an antigen that is not present in the assay system. A significant difference is observed for test antibody relative to a cognate isotype control within the dynamic range of the assay is indicative that the antibody acts as an agonist of the ICOS receptor in that assay.

An agonist antibody may be defined as one which, when tested in a T-cell activation assay:
has a significantly lower EC50 for induction of IFNγ production compared with control antibody;
induces significantly higher maximal IFNγ production compared with control antibody;
has a significantly lower EC50 for induction of IFNγ production compared with ICOSL-Fc;
induces significantly higher maximal IFNγ production compared with ICOSL-Fc;
has a significantly lower EC50 for induction of IFNγ production compared with reference antibody C398.4A; and/or
induces significantly higher maximal IFNγ production compared with reference antibody C398.4A.

A significantly lower or significantly higher value may for example be up to 0.5-fold different, up to 0.75-fold different, up to 2-fold different, up to 3-fold different, up to 4-fold different or up to 5-fold different, compared with the reference or control value.

Thus, in one example, an antibody (e.g. a multispecific antibody) provided herein has a significantly lower, e.g., at least 2-fold lower, EC50 for induction of IFNγ in an MJ cell activation assay using the antibody in bead-bound format, compared with control.

The bead-bound assay uses the antibody (e.g. multispecific antibody) (and, for control or reference experiments, the control antibody, reference antibody or ICOSL-Fc) bound to the surface of beads. Magnetic beads may be used, and various kinds are commercially available, e.g., Tosyl-activated DYNABEADS M-450 (DYNAL Inc, 5 Delaware Drive, Lake Success, N.Y. 11042 Prod No. 140.03, 140.04). Beads may be coated (coating methods are well-known to those skilled in the art), or generally by dissolving the coating material in carbonate buffer (pH 9.6, 0.2 M) or other method known in the art. Use of beads conveniently allows the quantity of protein bound to the bead surface to be determined with a good degree of accuracy. Standard Fc-protein quantification methods can be used for coupled protein quantification on beads. Any suitable method can be used, with reference to a relevant standard within the dynamic range of the assay. DELFIA, ELISA or other methods could be used.

Agonism activity of an antibody can also be measured in primary human T-lymphocytes ex vivo. The ability of an antibody (e.g. multispecific antibody) to induce expression of IFNγ in such T-cells is indicative of ICOS agonism. Preferably, an antibody will show significant (p<0.05) induction of IFNγ at 5 μg/mL compared with control antibody in a T-cell activation assay. An anti-ICOS antibody may stimulate T-cell activation to a greater degree than ICOS-L or C398.4 in such an assay. Thus, the antibody may show significantly (p<0.05) greater induction of IFNγ at 5 μg/mL compared with the control or reference antibody in a T-cell activation assay. TNFα or IL-2 induction may be measured as an alternative assay readout.

Agonism of an anti-ICOS antibody may contribute to its ability to change the balance between populations of TReg and TEff cells in vivo, e.g., in a site of pathology such as a tumour microenvironment, in favour of TEff cells. The ability of an antibody to enhance tumour cell killing by activated ICOS-positive effector T-cells may be determined, as discussed elsewhere herein.

Arrangement 8. A multispecific antibody according to any preceding arrangement, which binds (and optionally has specificity for) mouse ICOS and/or cynomolgus ICOS.

The multispecific antibodies described herein may be cross-reactive, and may for example bind the extracellular domain of mouse ICOS as well as human ICOS. The multispecific antibodies may bind other non-human ICOS, including ICOS of primates, such as cynomolgus monkey. An anti-ICOS multispecific antibody intended for therapeutic use in humans must bind human ICOS, whereas binding to ICOS of other species would not have direct therapeutic relevance in the human clinical context. Regardless of the underlying theory, however, cross-reactive antibodies are of high value and are excellent candidates as therapeutic molecules for pre-clinical and clinical studies. Cross-reactivity may be determined as set out for arrangement 2 hereinabove.

Arrangement 9. A multispecific antibody according to any preceding arrangement which is a bispecific antibody.

A bispecific antibody has any of the meanings set out hereinabove.

Arrangement 10. A bispecific antibody according to arrangement 9, wherein the bispecific antibody format is selected from DVD-Ig, mAb², FIT-Ig, mAb-dAb, dock and lock, SEEDbody, scDiabody-Fc, diabody-Fc, tandem scFv-Fc, Fab-scFv-Fc, Fab-scFv, intrabody, BITE, diabody, DART, TandAb, scDiabody, scDiabody-CH3, Diabody-CH3, minibody, knobs-in-holes, knobs-in-holes with common light chain, knobs-in-holes with common light chain and charge pairs, charge pairs, charge pairs with common light chain, in particular mAb², knob-in-holes, knob-in-holes with common light chain, knobs-in-holes with common light chain and charge pairs and FIT-Ig, e.g. mAb² and FIT-Ig.

In one embodiment, the bispecific antibody format is as described in any of concepts 37 to 40 described hereinabove, or as described in the definitions section. In one embodiment, the bispecific antibody format is a mAb², wherein the ICOS binding is provided by the Fcab portion of the bispecific antibody. In another embodiment, the bispecific antibody format is a mAb², wherein the ICOS binding is provided by the Fab portion of the bispecific antibody.

In another embodiment, the bispecific antibody is not a mAb² bispecific antibody.

Arrangement 11. A multispecific antibody according to any one of arrangements 1 to 8 which is a dual binding antibody.

A dual-binding antibody has any of the meanings set out hereinabove.

Arrangement 12. A multispecific, bispecific or dual binding antibody according to any one of arrangements 1 to 11, wherein the another target antigen is selected from immune checkpoint inhibitors, immune modulators and immune activators.

Arrangement 13. A multispecific, bispecific or dual-binding antibody according to arrangement 12, wherein the another target antigen is selected from PD-1, PD-L1, CTLA-4, TIGIT, TIM-3, LAG-3, VISTA, BTLA, HVEM, CSF1R, CCR4, CD39, CD40, CD73, CD96, CXCR2, CXCR4, CD200, GARP, SIRPα, CXCL9, CXCL10, CXCL11, CD155, CD137, GITR, OX40, CXCR3, CD27 and CD3.

In one embodiment, the antigen-binding site which binds the another target antigen is provided for by any of the CDRH1, CDRH2, CDR3, CDRL1, CDRL2 and CDRL3, or the VH, or the VL or the VH and VL regions from any one of the antibodies against the targets listed in arrangement 13.

Arrangement 13a. A multispecific, bispecific or dual-binding antibody according to arrangement 12, wherein the another target antigen is selected from PD-1, PD-L1, CTLA-4, TIGIT, TIM-3, LAG-3, VISTA, BTLA, HVEM, CSF1R, CCR4, CD39, CD40, CD73, CD96, CXCR2, CXCR4, CD200, GARP, SIRPα, CXCL9, CXCL10, CD155, CD137, GITR, OX40, CXCR3 and CD3.

Arrangement 14. A multispecific, bispecific or dual-binding antibody according to arrangement 13, wherein the another target antigen is selected from PD-L1, TIGIT, TIM-3, LAG-3, GARP, SIRPα, CXCR4, BTLA, HVEM, CSF1R, agonistic anti-CXCR3 antibodies), CD137, GITR and OX40.

Arrangement 15. A multispecific, bispecific or dual-binding antibody according to arrangement 14, wherein the another target antigen is PD-L1 (e.g. human PD-L1).

Arrangement 16. A multispecific, bispecific or dual-binding antibody according to arrangement 15, wherein the binding (and optionally specificity for) PD-L1 is provided by any of the antibodies or fragments as defined in concepts 1 to 70.

Arrangement 17. A multispecific, bispecific or dual-binding antibody according to arrangement 15 or arrangement 16, which comprises a VH domain comprising a CDRH1, a CDRH2 and a CDRH3 which VH domain has specificity for human PD-L1.

Arrangement 18. A multispecific, bispecific or dual-binding antibody according to any one of arrangements 15 to 17, which comprises a VL domain comprising a CDRL1, a CDRL2 and a CDRL3, which VL domain as specificity for human PD-L1.

Arrangement 19. A multispecific, bispecific or dual-binding antibody according to arrangement 17 or arrangement 18, wherein the VH and/or VL domain is any of VH and/or VL domains from atezolizumab (Roche), avelumab (Merck), BMS-936559 (BMS), durvalumab (Medimmune) or from any of the PD-L1 antibodies disclosed in WO2016/061142, WO2016/022630, WO2016/007235, WO2015/173267, WO2015/181342, WO2015/109124, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959 or WO2007/005874.

Arrangement 20. A multispecific, bispecific or dual-binding antibody according to arrangement 17 or arrangement 18, wherein the VH and/or VL domain is any of VH and/or VL domains described in concepts 1 to 70.

Arrangement 21. A multispecific, bispecific or dual-binding antibody according to any one of arrangements 15 to 20, which binds (and optionally has specificity for) mouse PD-L1 and/or cynomolgus PD-L1.

Cross reactivity may be as described hereinabove for arrangement 2 or concept 27.

Arrangement 22. A composition comprising a multispecific, bispecific or dual-binding antibody as defined in any preceding arrangement and a pharmaceutically acceptable excipient, diluent or carrier and optionally further comprising a further therapeutic agent independently selected from the group consisting of:
a) other immune checkpoint inhibitors (such as anti-TIM-3 antibodies, anti-PD-1 antibodies, anti-CTLA-4 antibodies, anti-TIGIT antibodies and anti-LAG-3 antibodies);
b) immune stimulators (such as anti-OX40 antibodies, anti-GITR antibodies, anti-CD137 antibodies, anti-ICOS antibodies and anti-CD40 antibodies);
c) chemokine receptor antagonists (such as CXCR4, CCR4 and CXCR2);
d) targeted kinase inhibitors (such as CSF-1R or VEGFR inhibitors);
e) angiogenesis inhibitors (such as anti-VEGF-A or Delta-like Ligand-4);
f) immune stimulating peptides or chemokines (such as CXCL9 or CXCL10);
g) cytokines (such as IL-15 and IL-21);
h) bispecific T-cell engagers (BiTEs) having at least one specificity against CD3 (e.g. CD3/CD19 BiTE);
i) other bi-specific molecules (for example IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
j) oncolytic viruses (such as HSV virus (optionally which secretes GMCSF), Newcastle disease virus and Vaccinia virus);
k) vaccination with tumour associated antigens (such as New York Esophageal Cancer-1 [NY-ESO-1], Melanoma Associated Antigen-3 [MAGE-3]);
l) cell-based therapies (such as chimeric Antigen Receptor-T-cells (CAR-T) for example expressing anti-CD19, anti-EpCam or anti-mesothelin);
m) bi-specific NK cell engagers having a specificity against an activating MK receptor such as NKG2D or CD16a; and
n) adoptive transfer of tumour specific T-cells or LAK cells.

The antibodies may be any of the sequences or antibodies described in arrangement 5. Other features of this arrangement may be as described in concept 49.

Arrangement 22a. A pharmaceutical composition according to arrangement 22, or a kit comprising a pharmaceutical composition as defined in arrangement 22, wherein the composition is for treating and/or preventing a condition or disease selected from neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease, diffuse large B-cell lymphoma.

Arrangement 22b. A pharmaceutical composition according to arrangement 22 or arrangement 22a in combination with, or kit according to arrangement 22a comprising, a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the multispecific, bispecific or dual-binding antibody.

Arrangement 23. A multispecific, bispecific or dual-binding antibody as defined in any one of arrangements 1 to 21 for use in treating or preventing a disease or condition, selected from neurological disease, neoplastic or non-neoplastic disease, chronic viral infections, and malignant tumours; such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

Arrangement 24. Use of a multispecific, bispecific or dual-binding antibody as defined in any one of arrangements 1 to 21 in the manufacture of a medicament for administration to a human for treating or preventing a disease or condition in the human selected from neurological disease, neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas).

Arrangement 25. A method of treating or preventing a disease or condition selected from neurological disease, neoplastic or non-neoplastic disease, chronic viral infections, and malignant tumours, such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas) in a human, comprising administering to said human a therapeutically effective amount of a multispecific, bispecific or dual-binding antibody as defined in any one of arrangements 1 to 21, wherein the disease or condition is thereby treated or prevented.

The diseases and conditions which may be treated or prevented by the multispecific, bispecific or dual-binding antibodies provided for in these arrangements may be any of the diseases provided for in, for example concepts 41 to 45 or in any of the sentences described herein.

Arrangement 26. The multispecific, bispecific or dual-binding antibody according to arrangement 23, the use according to arrangement 24 or the method according to arrangement 25, wherein the neurological disease is a neurodegenerative disease, disorder or condition, optionally wherein the neurodegenerative disease, disorder or condition is selected from Alzheimer's disease, amyotrophic lateral sclerosis, Parkinson's disease, Huntington's disease, primary progressive multiple sclerosis, secondary progressive multiple sclerosis, corticobasal degeneration, Rett syndrome, a retinal degeneration disorder selected from age-related macular degeneration and retinitis pigmentosa; anterior ischemic optic neuropathy, glaucoma, uveitis, depression, trauma-associated stress or post-traumatic stress disorder, frontotemporal dementia, Lewy body dementias, mild cognitive impairments, posterior cortical atrophy, primary progressive aphasia and progressive supranuclear palsy or aged-related dementia, in particular Alzheimer's disease, amyotrophic lateral sclerosis, Parkinson's disease and Huntington's disease, and e.g. Alzheimer's disease.

Arrangement 27. The multispecific, bispecific or dual-binding antibody according to arrangement 23, the use according to arrangement 24 or the method according to arrangement 25, wherein the cancer is selected from melanoma, Merkel cell carcinoma, non-small cell lung cancer (squamous and non-squamous), renal cell cancer, bladder cancer, head and neck squamous cell carcinoma and mesothelioma or is selected from virally induced cancers (such as cervical cancer and nasopharyngeal cancer) and soft tissue sarcomas.

Arrangement 28. The multispecific, bispecific or dual-binding antibody, the use or the method according to any one of arrangements 23 to 27, further comprising administering to the human a further therapy, for example a further therapeutic agent, optionally wherein the further therapeutic agent is independently selected from the group consisting of:
a. other immune checkpoint inhibitors (such as anti-TIM-3 antibodies, anti-PD-1 antibodies, anti-CTLA-4 antibodies, anti-TIGIT antibodies and anti-LAG-3 antibodies);
b. immune stimulators (such as anti-OX40 antibodies, anti-GITR antibodies, anti-CD137 antibodies and anti-CD40 antibodies);
c. chemokine receptor antagonists (such as CXCR4, CCR4 and CXCR2);
d. targeted kinase inhibitors (such as CSF-1R or VEGFR inhibitors);
e. angiogenesis inhibitors (such as anti-VEGF-A or Delta-like Ligand-4);
f. immune stimulating peptides or chemokines (such as CXCL9 or CXCL10);
g. cytokines (such as IL-15 and IL-21);
h. bispecific T-cell engagers (BiTEs) having at least one specificity against CD3 (e.g. CD3/CD19 BiTE);
i. other bi-specific molecules (for example IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
j. oncolytic viruses (such as HSV virus (optionally which secretes GMCSF), Newcastle disease virus and Vaccinia virus);
k. vaccination with tumour associated antigens (such as New York Esophageal Cancer-1 [NY-ESO-1], Melanoma Associated Antigen-3 [MAGE-3]);
l. cell-based therapies (such as chimeric Antigen Receptor-T-cells (CAR-T) for example expressing anti-CD19, anti-EpCam or anti-mesothelin);
m. bi-specific NK cell engagers having a specificity against an activating MK receptor such as NKG2D or CD16a; and
n. adoptive transfer of tumour specific T-cells or LAK cells, or optionally wherein the further therapy is chemotherapy, radiotherapy and surgical removal of tumours. Radiotherapy may be single dose or in fractionated doses, either delivered to affected tissues directly or to the whole body.

In this arrangement, any of the features and embodiments of concept 46 apply mutatis mutandis.

In this aspect, the bispecific molecules include “bispecific antibodies” and antibody fusion proteins, including those formats and molecules described in concepts 37 to 40.

Arrangement 29. A nucleic acid that encodes a heavy chain and/or a light chain of a multispecific, bispecific or dual-binding antibody as defined in any one of arrangements 1 to 21.

Arrangement 30. A vector comprising the nucleic acid as defined in arrangement 29; optionally wherein the vector is a CHO or HEK293 vector.

Arrangement 31. A host comprising the nucleic acid as defined in arrangement 29 or the vector as defined in arrangement 30.

Multispecific antibodies that bind ICOS and PD-L1 may be used in any of the medical treatment methods described herein with reference to anti-PD-L1 antibodies, and may be manufactured and formulated as described with reference to anti-PD-L1 antibodies.

### Therapeutic Uses for Anti-PD-L1 Antibodies

In one embodiment, the PD-L1 specific antibodies described herein and antigen binding fragments thereof can be used for therapeutic modulation of the PD-1/PD-L1 pathway. In one embodiment, the PD-L1 specific antibody or fragment thereof is as described in any concept, aspect or embodiment herein.

In one embodiment, the antibody or antibody binding fragment specifically binds to PD-L1 and thereby inhibits PD-L1 activity. In another embodiment, the antibody or antibody binding fragment specifically binds to PD-L1 and thereby inhibits binding of PD-L1 to PD-1. In another embodiment, the antibody or antibody binding fragment specifically binds to PD-L1 and thereby inhibits binding of PD-L1 to B7-1. In yet another embodiment, the antibody or antigen binding fragment thereof blocks PD-L1 induced T-cell suppression and thereby enhance anti-tumour immunity.

In yet another embodiment, the antibody or antigen binding fragment thereof is capable of stimulating one or more of the following activities: T-cell proliferation, IFN-γ, CD25 and/or IL-2 secretion in mixed lymphocyte reactions.

In one embodiment, the antibody or antigen binding fragment thereof specifically binds PD-L1 and inhibits PD-L1 induced cell proliferation, for example, tumour cell proliferation and/or inhibits tumour cell survival. In another embodiment, the antibody or antigen binding fragment thereof specifically binds PD-L1 and thereby inhibits PD-L1 mediated suppression of T-cells, including, but not limited to, tumour reactive T-cells, thereby enhancing anti-tumour cytolytic T-cell activity. In other embodiments, the antibodies or binding fragments thereof as described herein inhibit tumour cell adhesion, motility, invasion and cellular metastasis, and reduce tumour growth. In other embodiments, the antibodies or binding fragments thereof can bind to cells expressing PD-L1, including tumour and non-tumour cells, and recruit, by means of interaction with the Fc portion of the antibody, cellular effector functions against the target cells by mechanisms including but not limited to antibody dependent cellular cytotoxicity (ADCC) and antibody dependent cellular phagocytosis (ADCP).

Still further embodiments include methods of treating a proliferative or invasion-related disease in a mammal by administering to the animal a therapeutically effective dose of an antibody or antigen binding fragment thereof. In another embodiment, the antibodies or antigen binding fragments thereof can be used in a method for treating a mammal suffering from a disease selected from: neoplastic or non-neoplastic disease, chronic viral infection, and a malignant tumour, wherein the method includes administering to the mammal a therapeutically effective dose of an antibody or antigen binding fragment thereof.

Still further embodiments include methods of treating a disease of immunological dysfunction in a mammal by administering to the animal a therapeutically effective dose of an antibody or antigen binding fragment thereof as described herein. Exemplary immunological dysfunction in humans includes diseases of neurological deficit, such as Alzheimer's disease.

It has further been proposed that an immune response, particularly an IFNγ-dependent systemic immune response, could be beneficial for treatment of Alzheimer's disease and other CNS pathologies that share a neuroinflammatory component. WO2015/136541 proposes treatment of Alzheimer's disease using an anti-PD-1 antibody (also see Baruch K. et al., PD-1 immune checkpoint blockade reduces pathology and improves memory in mouse models of Alzheimer's disease, Nature Medicine, 2016, 22(2):137-137).

Thus, the PD-L1 mediated disease or condition is a neurodegenerative disease, disorder or condition. In one embodiment, the neurodegenerative disease, disorder or condition is Alzheimer's disease. In another embodiment, the neurodegenerative disease, disorder or condition is selected from amyotrophic lateral sclerosis, Parkinson's disease, Huntington's disease, primary progressive multiple sclerosis, secondary progressive multiple sclerosis, corticobasal degeneration, Rett syndrome, a retinal degeneration disorder selected from age-related macular degeneration and retinitis pigmentosa; anterior ischemic optic neuropathy, glaucoma, uveitis, depression, trauma-associated stress or post-traumatic stress disorder, frontotemporal dementia, Lewy body dementias, mild cognitive impairments, posterior cortical atrophy, primary progressive aphasia and progressive supranuclear palsy or aged-related dementia. In another embodiment, the neurodegenerative disease, disorder or condition is selected from Alzheimer's disease, amyotrophic lateral sclerosis, Parkinson's disease and Huntington's disease.

Anti-PD-L1 antibodies as described herein may be used in the treatment of Alzheimer's disease or other neurodegenerative diseases, optionally in combination with one or more other immune checkpoint inhibitors (such as anti-TIM-3 antibodies, anti-CTLA-4 antibodies, anti-TIGIT antibodies and anti-LAG-3 antibodies) or one or more other immune stimulators (such as anti-OX40 antibodies, anti-GITR antibodies, anti-CD137 antibodies, anti-ICOS antibodies and anti-CD40 antibodies, including those which are specifically described in Aspect 1a herein). Other combination partners include any of the active agents as listed in claim 10 of WO2015/136541, which is incorporated herein by reference.

Any of the PD-L1 antibodies described herein (including at least the antibodies described in any of concepts 1 to 40) may be used for the treatment of the neurodegenerative diseases, disorders or conditions described above.

Exemplary cancers in humans include a Merkel cell carcinoma, breast cancer, prostate cancer, basal cell carcinoma, biliary tract cancer, bladder cancer, bone cancer, brain and CNS cancer (e.g. glioblastoma), cervical cancer, choriocarcinoma, colon and rectum cancer, connective tissue cancer, cancer of the digestive system; endometrial cancer, esophageal cancer; eye cancer; cancer of the head and neck; nasopharyngeal cancer; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma including but not limited to DLBCL; Chronic lymphocytic leukaemia, melanoma; uveal melanoma, myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer (renal cell carcinoma (RCC)), cancer of the respiratory system; sarcoma, skin cancer; stomach cancer, testicular cancer, thyroid cancer; uterine cancer, cancer of the urinary system, as well as other carcinomas and sarcomas. Further examples of virally induced cancers including; Nasopharyngeal carcinoma, certain Types of NHL (for example but not limited to EBV+CNS lymphomas, DLBCL and BL, Hodgkins lymphoma (thought to be EBV driven) HPV-related cervical and head an neck squamous cell carcinomas); HBV hepatocellular carcinoma.

Exemplary chronic infections in humans include HIV, hepatitis B virus (HBV), and hepatitis C virus (HCV).

Proliferative or invasion-related diseases that can be treated with the antibodies or antigen binding fragments described herein include neoplastic diseases, and the metastasis associated with such neoplastic disease, such as, melanoma, uveal melanoma, skin cancer, small cell lung cancer, non-small cell lung cancer, salivary gland, glioma, hepatocellular (liver) carcinoma, gallbladder cancer, thyroid tumour, bone cancer, gastric (stomach) cancer, prostate cancer, breast cancer (including triple negative breast cancer), ovarian cancer, cervical cancer, uterine cancer, vulval cancer, endometrial cancer, testicular cancer, bladder cancer, lung cancer, glioblastoma, thyroid cancer, endometrial cancer, kidney cancer, colon cancer, colorectal cancer, pancreatic cancer, esophageal carcinoma, brain/CNS cancers, neuronal cancers, head and neck cancers (including but not limited to squamous cell carcinoma of the head and neck (SCCHN)), mesothelioma, sarcomas, biliary (cholangiocarcinoma), small bowel adenocarcinoma, pediatric malignancies, epidermoid carcinoma, sarcomas, cancer of the pleural/peritoneal membranes and leukaemia, including acute myeloid leukaemia, acute lymphoblastic leukaemia, and multiple myeloma. Treatable chronic viral infections include HIV, hepatitis B virus (HBV), and hepatitis C virus (HCV) in humans, simian immunodeficiency virus (SIV) in monkeys, and lymphocytic choriomeningitis virus (LCMV) in mice.

The antibody or antigen binding fragment thereof can be administered alone, or in combination with other antibodies or chemo therapeutic drugs, radiation therapy or therapeutic vaccines. In one embodiment, the antibody or antigen binding fragment thereof is administered as an antibody-drug conjugate in which the antibody or antigen binding fragment thereof is linked to a drug moiety such as a cytotoxic or cytostatic agent. The use of antibody-drug conjugates for the local delivery of cytotoxic or cytostatic agents in the treatment of cancer allows targeted delivery of the drug moiety to tumours, and intracellular accumulation therein, where systemic administration of unconjugated drug may result in unacceptable levels of toxicity. Drugs in antibody drug conjugates can include, but are not limited to, daunomycin, doxorubicin, methotrexate, and vindesine. Toxins can also be used in antibody-toxin conjugates, including, for example, bacterial toxins such as diphtheria toxin, plant toxins such as ricin, small molecule toxins such as geldanamycin. The toxins may effect their cytotoxic and cytostatic effects by mechanisms including tubulin binding, DNA binding, or topoisomerase.

### Pharmaceutical Compositions and Formulations of Anti-PD-L1 Antibodies

In one embodiment, there is provided a pharmaceutical composition comprising an effective amount of an antibody or antigen binding fragment and a pharmaceutically acceptable carrier. An effective amount of antibody to be employed therapeutically will depend, for example, upon the therapeutic objectives, the route of administration, and the condition of the patient. In one embodiment, the composition includes other excipients or stabilizers.

Pharmaceutically acceptable carriers are known and include carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or nonionic surfactants such as TWEEN™, polyethylene glycol (PEG), and PLURONICS™

The antibodies or antigen binding fragments can be administered intravenously or through the nose, lung, for example, as a liquid or powder aerosol (lyophilized). The composition can also be administered parenterally or subcutaneously. When administered systemically, the composition should be sterile, pyrogen-free and in a physiologically acceptable solution having due regard for pH, isotonicity and stability. These conditions are known to those skilled in the art.

Methods of administering a prophylactic or therapeutic agent (e.g., an antibody as disclosed herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody as disclosed herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration. In one embodiment, an anti-PD-L1 antibody or fragment as disclosed herein may be administered via multiple routes of administration simultaneously or subsequently to other doses of the same or a different anti-PD-L1 antibody or fragment as disclosed herein.

Various delivery systems are known and can be used to administer a prophylactic or therapeutic agent (e.g., an antibody or fragment as disclosed herein), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO92/19244, WO97/32572, WO97/44013, WO98/31346, and WO99/66903, each of which is incorporated herein by reference their entirety.

In a specific embodiment, it may be desirable to administer a prophylactic or therapeutic agent, or a pharmaceutical composition as described herein locally to the area in need of treatment. This may be achieved by, for example, local infusion, by topical administration (e.g., by intranasal spray), by injection, or by means of an implant, said implant being of a porous, non-porous, or gelatinous material, including membranes, such as sialastic membranes, or fibres. When administering an anti-PD-L1 antibody or fragment, care must be taken to use materials to which the antibody does not absorb.

In another embodiment, a kit for treating diseases involving the expression of PD-L1 is provided, wherein the kit includes an antibody or antigen binding fragment described herein and instructions to administer the antibody or antigen binding fragment to a subject in need of treatment. There is also provided a pharmaceutical or diagnostic pack or kit comprising one or more containers filled with one or more of the ingredients of the pharmaceutical compositions as disclosed herein, such as one or more anti-PD-L1 antibodies or fragments provided herein. Optionally associated with such container(s) can be a notice in the form prescribed by a governmental agency regulating the manufacture, use or sale of pharmaceuticals or biological products, which notice reflects approval by the agency of manufacture, use or sale for human administration, e.g., an authorisation number. In another embodiment, an article of manufacture that includes a container in which a composition containing an antibody or antigen binding fragment described herein and a package insert or label indicating that the composition can be used to treat diseases characterized by the expression or overexpression of PD-L1 is provided. In one embodiment, there is provided a kit for treating and/or preventing a PD-L1-mediated condition or disease, the kit comprising an antibody or fragment as disclosed herein in any embodiment or combination of embodiments (and optionally a further therapeutic agent as described elsewhere herein) optionally in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody or fragment. In another embodiment, the kit comprises an antibody or antigen binding fragment thereof contained within a container or an IV bag. In another embodiment, the container or IV bag is a sterile container or a sterile IV bag. In another embodiment, the antibody or antigen binding fragment therefore is formulated into a pharmaceutical composition contained within a (sterile) container or contained within a (sterile) IV bag. In a further embodiment, the kit further comprises instructions for use.

### EXAMPLES

A number of bispecific antibodies were generated, using different antibody formats, and with different anti-ICOS and anti-PD-L1 binding sites. Bispecific antibodies were shown to bind and induce an agonistic signal at ICOS, to bind to PD-L1 inhibiting the interaction with PD-1, and to deplete cells expressing high levels of ICOS.

Anti-ICOS Fv regions of antibodies, and anti-PD-L1 Fv regions of antibodies, can be generated using the Kymouse—a transgenic mouse technology platform. Kymouse covers the entire human immunoglobulin (lg) repertoire of V, D and J genes required to make fully human antibodies. On selection and recovery of recombined variable regions, the antibodies are re-formatted to yield antibodies with isotypes or Fc-domains of choice. STIM001 and STIM003 are anti-ICOS antibodies originating from the Kymouse. Their Fv regions were included in bispecific antibody formats as described in these Examples.

The example anti-PD-L1 binding domains that were used in the mAb² bispecific antibodies described in these Examples were produced as Fcabs, utilising permissive residues in the CH3 domain of the constant chain of human IgG1 termed the AB, CD and EF loops to generate IgG-based Fcabs which bind PD-L1.

### Example 1 Generation of ICOS/PD-L1 Bispecific Antibody

### FIT-Ig

Bispecific antibodies for ICOS and PD-L1 were generated in FIT-Ig format, as illustrated in FIG. 2.

The anti-PD-L1/ICOS tetravalent bispecific FIT-Ig molecule combines the variable regions of an anti-ICOS antibody with the variable regions of an anti-PD-L1 antibody. The bispecific molecule presents two Fabs in tandem, fused with an Fc. The molecules are symmetrical.

In this example, the anti-ICOS antibody domains were those of STIM003 and the anti-PD-L1 antibody domains were those of Antibody W. The FIT-Ig molecules were generated with anti-ICOS binding specificity as the “outer” Fab (antibody A in FIG. 2) or with anti-ICOS binding specificity as the “inner” Fab (antibody B in FIG. 2). Fc domains were either human IgG1 or mouse IgG2a. Thus a total of four different FIT-Ig molecules were produced using the STIM003 and AbW binding specificities:
ICOS/PD-L1 hIgG1
ICOS/PD-L1 mIgG2a
PD-L1/ICOS hIgG1
PD-L1/ICOS mIgG2a

For each FIT-Ig molecule, the three constructs shown in FIG. 2(ii) were generated, and cloned into an expression vector (pTT5) via restriction enzymes and sequences were verified. A 30 mL HEK transient transfection was then performed, using the three constructs in 1:1:1 ratio. After 6 days, supernatant was analysed to assess the level of expression, quantifying using human IgG1 and mouse IgG2a as standards. Expression data are shown in Table E1-1 below.

TABLE E1-1
Expression of FIT-Ig in μg/mL.

| | | |
|---|---|---|
| Standard human IgG1 | STIM003_AbW_hIgG1 | 11.1 |
| | AbW_STIM003_hIgG1 | 52.5 |
| Standard mouse IgG2a | STIM003_AbW_mIgG2a | 44.2 |
| | AbW_STIM003_mIgG2a | 42.8 |

Sequences of further example FIT-Ig molecules are shown in Table S3.

mAb²

Bispecific antibodies for ICOS and PD-L1 were generated in mAb² format, as illustrated in FIG. 3. The following molecules were generated and expressed:

| | |
|---|---|
| STIM001_289. | A mAb² IgG1 in which the two Fab regions comprise the |
| | VH and VL domains of anti-ICOS antibody STIM001 and |
| | the Fcab region binds human PD-L1. |
| STIM001_457. | A mAb² IgG1 in which the two Fab regions comprise the |
| | VH and VL domains of anti-ICOS antibody STIM001 and |
| | the Fcab region binds mouse PD-L1. |
| STIM003 289. | A mAb² IgG1 in which the two Fab regions comprise the |
| | VH and VL domains of anti-ICOS antibody STIM003 and |
| | the Fcab region binds human PD-L1. |
| STIM003_457. | A mAb² IgG1 in which the two Fab regions comprise the |
| | VH and VL domains of anti-ICOS antibody STIM003 and |
| | the Fcab region binds mouse PD-L1. |

The following control antibodies were also generated for use in the experiments:

| | |
|---|---|
| IgG1_289. | A mAb² IgG1 in which the two Fab regions do not bind |
| | ICOS or PD-L1, and the Fcab region binds human PD-L1. |
| IgG1_457. | A mAb² IgG1 in which the two Fab regions do not bind |
| | ICOS or PD-L1, and the Fcab region binds mouse PD-L1. |
| IgG1_438. | A mAb² IgG1 in which the two Fab regions do not bind |
| | ICOS or PD-L1, and the Fcab region binds mouse PD-L1. |
| | IgG1_438 and IgG1_457 have minor variation in the |
| | amino acid sequences of the antigen-binding loops |
| | in the Fcab and are functionally equivalent for the |
| | purposes of the assays described below. |

The mAb² antibodies were generated as IgG1, containing IgG1 CH1, CH2 and CH3 constant regions, with the Fcab binding loops in the IgG1 CH3 constant region. Unless otherwise stated, IgG1 was wild type human IgG1. The “LAGA” variant IgG1 sequence was used where specified. The “LAGA” variant includes mutations L235A and G237A which disable Fc-mediated effects ADCC and CDC as described in WO99/58679 and in Shields et al. J. Biol. Chem., March 2; 276(9):6591-604 2001.

### Example 2 Kinetic Surface Plasmon Resonance (SPR) Assay for Characterisation of Bispecific Antibody Binding to ICOS and PD-L1

Analysis of mAb² Binding to Human PD-L1 and Mouse PD-L1

This kinetic SPR assay confirmed that addition of human variable regions to anti-PD-L1 Fcab molecules to create mAb² constructs did not affect the ability of the Fcab to recognise PD-L1. Six different mAb²_289 constructs exhibited similar binding to human PD-L1, and six different mAb²_457 constructs exhibited similar binding to mouse PD-L1.

### Method:

An anti-human IgG capture surface was created on a Series S C1 chip (GE Healthcare, cat No BR100535). A cocktail of three anti-human IgG antibodies were covalently coupled to the biosensor chip surface (Jackson Labs: cat No 109-005-008; cat No 309-006-008 and cat No 109-006-008), using 10 mM Na Acetate pH 4.5 buffer as the diluent for the antibody.

For the kinetic assay, mAb² constructs were diluted to 5 μg/mL in running buffer (1×HBS-EP+Buffer Technova, cat. No. H8022) and captured on the anti-human capture surface. The human recombinant extra cellular domain PD-L1 protein was used as analyte at 81 nM, 27 nM, 9 nM, 3 nM, 1 nM and 0 nM, and injected over the mAb²_289 constructs. The mouse recombinant extra cellular domain PD-L1 protein was injected as analyte at 243 nM, 81 nM, 27 nM, 9 nM, 3 nM, 1 nM and 0 nM over the mAb²_457 constructs. Finally, the surface was regenerated between each mAb² construct using 100 mM PO₄. The assay was carried out at 25° C.

The buffer injection (i.e. 0 nM) was used to double reference the sensorgrams. The analysis was carried out using the 1:1 binding model inherent to the Biacore 8K's analysis software.

### Results

Data for binding to human PD-L1 are shown in Table E2-1 below.

TABLE E2-1
mAb²_289 affinities for binding to human PD-L1. Five concentrations
of human PD-L1 (81, 27, 9, 3 and 1 nM) were used as analyte over
each mAb²_289 construct, captured at 5 μg/mL.

| | ka (1/Ms) | kd (1/s) | KD (nM) |
|---|---|---|---|
| hybrid control_289 | 8.99E+06 | 1.84E−03 | 0.20 |
| hybrid control_289_LAGA | 8.14E+06 | 1.40E−03 | 0.17 |
| STIM003_289 | 5.31E+06 | 1.38E−03 | 0.26 |
| STIM003_289_LAGA | 8.69E+06 | 1.63E−03 | 0.19 |
| STIM001_289 | 8.94E+06 | 1.87E−03 | 0.21 |
| STIM001_289_LAGA | 7.68E+06 | 1.47E−03 | 0.19 |

With respect to the Ko values, the binding of the mAb²_289 constructs to human PD-L1 was comparable and within the variance expected with this type of assay.

Data for binding to mouse PD-L1 are shown in Table E2-2 below.

TABLE E2-2
mAb²_457 affinities for binding to mouse PD-L1. Six concentrations
of mouse PD-L1 (243, 81, 27, 9, 3 and 1 nM) were used as analyte over
each mAb²_457 construct, captured at 5 μg/mL.

| | ka (1/Ms) | kd (1/s) | KD (nM) |
|---|---|---|---|
| STIM003_457 | 1.31E+06 | 1.07E−01 | 82.0 |
| STIM003_457_LAGA | 1.36E+06 | 1.30E−01 | 95.9 |
| STIM001_457 | 1.32E+06 | 1.29E−01 | 97.8 |
| STIM001_457_LAGA | 2.11E+06 | 2.27E−01 | 107.8 |
| hybrid control_457 | 1.35E+06 | 1.34E−01 | 99.3 |
| hybrid control_457_LAGA | 1.32E+06 | 1.43E−01 | 108.7 |

With respect to the Ko values, the binding of the mAb²_457 constructs to mouse PD-L1 is comparable. However, given that the apparent affinity of the interaction is in the 80-110 nM range and the top concentration of mouse recombinant extra cellular domain PD-L1 was only 243 nM, it is unlikely that a true saturated Rmax was achieved in this assay, hence the actual affinity may be lower than indicated by these data. Nevertheless it can be concluded that the constructs are comparable in their binding and the anti-PD-L1 Fcab retains its binding for mouse PD-L1 when incorporated into a mAb² format.

Avidity Surface Plasmon Resonance Analysis of mAb² Binding to Human ICOS

This avidity SPR assay confirmed that the bispecific mAb² constructs were able to bind ICOS. Values obtained for binding were similar across all samples tested and within experimental variance for this type of assay, where capture level, concentration and biophysical form can have an impact on values.

### Method:

Biotinylated human ICOS protein diluted at 7.5 μg/mL in running buffer (1×HBS-EP+Buffer Technova, cat. No. H8022) was captured on a NLC sensor chip (Bio-Rad, cat No 1765021). The surface was then blocked using biocytin (Sigma Aldrich, cat No B1758) at 1 mg/mL.

The ICOS/PD-L1 bispecific mAb² and the corresponding human anti-ICOS IgG1 constructs were injected as analyte at 500, 167, 56, 18.5 and 0 nM for the STIM001_mAb² and STIM001, and at 40, 10, 2.5, 0.625 and 0 nM for the STIM003_mAb² and STIM003. The assay was carried out at 25° C.

The buffer injection (i.e. 0 nM) was used to double reference the sensorgrams. The analysis was carried out using the equilibrium model inherent to the ProteOn's analysis software for each mAb² construct.

### Results

Data for STIM003 mAb² constructs binding to human ICOS are shown in Table E2-3.

TABLE E2-3
Apparent affinities of STIM003_mAb² for binding to human ICOS.
Four concentrations of STIM003_mAb² and STIM003 (40, 10, 2.5
and 0.625 nM) were used as analyte over the human ICOS protein,
captured at 7.5 μg/mL.

| | | Apparent | | |
|---|---|---|---|---|
| | Capture | KD (nM) | | Calculated |
| | level | from | Off-rate | On-rate |
| mAb² | (RU) | equilibrium | (1/s) | (1/M · s) |
| ST1M003_289 | 528 | 0.7 | 2.65E−04 | 3.55E+05 |
| STIM003_289_LAGA | 548 | 0.6 | 2.78E−04 | 4.77E+05 |
| ST1M003_457 | 1122 | 0.9 | 2.44E−04 | 2.75E+05 |
| STIM003_457_LAGA | 935 | 0.8 | 2.32E−04 | 2.84E+05 |
| STIM003 | 543 | 1.7 | 4.49E−04 | 2.67E+05 |

Data for STIM001 mAb² constructs binding to human ICOS are shown in Table E2-4.

TABLE E2-4
Apparent affinities of STIM001_mAb² for binding to human ICOS.
Four concentrations of STIM001_mAb² and STIM001 (500, 167,
56 and 18.5 nM) were used as analyte over the human ICOS protein,
captured at 7.5 μg/mL.

| | | Apparent | | |
|---|---|---|---|---|
| | Capture | KD (nM) | | Calculated |
| | level | from | Off-rate | On-rate |
| mAb² | (RU) | equilibrium | (1/s) | (1/M · s) |
| STIM001_289 | 712 | 19.1 | 4.18E−04 | 2.19E+04 |
| STIM001_289_LAGA | 1166 | 25.4 | 3.54E−04 | 1.39E+04 |
| STIM001_457 | 1011 | 9.2 | 3.46E−04 | 3.76E+04 |
| STIM001_457_LAGA | 716 | 10.6 | 3.32E−04 | 3.13E+04 |
| STIM001 | 730 | 57.1 | 4.56E−04 | 7.99E+03 |

In conclusion, these data indicate that the presence of the PD-L1 binding site in the bispecific molecule does not affect binding of the anti-ICOS Fab arms.

### Example 3 ELISA Characterisation of Bispecific Antibody Binding to ICOS and PD-L1

Antibodies in mAb² format were assessed for binding to recombinant human ICOS, mouse ICOS, human PD-L1 and mouse PD-L1 proteins. Binding of the ICOS/PD-L1 bispecific mAb² antibodies to recombinant ICOS protein and recombinant PD-L1 protein was confirmed in this assay.

### DELFIA ELISA Method:

Recombinant ICOS proteins, human ICOS-mFc or mouse ICOS-mFc (Chimerigen) were diluted in 1×PBS and added to Black Hi-bind plates (Griener) at 1 μg/ml, 50 μl/well. Recombinant PD-L1 proteins, human PD-L1-Flag-His or mouse-His were diluted in 1×PBS and added to Black Hi-bind plates (Griener) at 4 μg/ml, 50 μl/well. The plates were left overnight at 4° C. The next day plates were washed 3× with 300 μl/well of 1×PBS+0.1% Tween and blocked with 200 μl/well of 1×PBS+1% BSA blocking buffer for 1 hr at RT on a plate shaker. Plates were washed 3× with 300 μl/well of 1×PBS+0.1% Tween.

In general, antibodies in monoclonal and mAb² format were diluted in 1×PBS+0.1% BSA buffer and diluted 1 in 3 from starting working concentration of either 0.399 μM, 0.199 μM over a 11 point titration. However, human-PD-L1 mAb² antibodies were diluted in 1×PBS+0.1% BSA buffer and diluted 1 in 2 from a starting working concentration of 0.066 μM over an 11 point titration. Titrated antibodies were added to the plates, 50 μl/well and left to incubate for 1 hr at R.T on a plate shaker. Plates were washed 3× with 300 μl/well of 1×PBS+0.1% Tween.

DELFIA® Eu-labelled Anti-human IgG(Perkin-Elmer) was diluted 1:500 in DELFIA Assay buffer (Perkin-Elmer) and added to the assay plate (50 μl/well), left to incubate for 1 hr at RT on a plate shaker. Plates were then washed 3× with 300 μl/well 1×DELFIA wash buffer before the addition of 50 μl/well of DELFIA Enhancement Solution (Perkin-Elmer), incubated for a minimum of 5 minutes in the dark. After incubation, assay was read on Envision plate reader (Perkin Elmer), Time-resolved fluorescence (TRF) was measured at 615 nm.

Titration curves and EC50 values [M] were plotted using Graphpad (Prism). EC50 values were calculated by first transforming the data using equation X=Log(X). The transformed data was then fitted using nonlinear regression, using fitting algorithm, log (agonist) vs. response-variable slope (four parameters).

### Results

Data are summarised in Tables E3-1 to E3-4 and shown in FIG. 4.

In the human ICOS ELISA assay STIM003_289 and STIM003_457 produced similar EC50 values to STIM003 (mean EC50 values; 0.63 nM±0.18 nM and 0.43±0.069 nM and 0.75±0.27 nM respectively).

In the human ICOS ELISA assay STIM001_289 and STIM001_457 produced similar

EC50 values to STIM001 (mean EC50 values; 13.1 nM±6.5 nM and 12.5±3.12 nM and 28.9±11 nM respectively).

In the mouse ICOS ELISA assay STIM003_289 and STIM003_457 produced similar EC50 values to STIM003 (mean EC50 values; 0.42 nM±0.075 nM and 0.28±0.037 nM and 0.37±0.039 nM respectively).

STIM001_289 and STIM001_457 produced similar EC50 values in the human ICOS ELISA assay (mean EC50 values; 13.16 nM±6.50 nM and 12.55 nM±3.12 nM respectively).

STIM003_289 and STIM003_457 produced similar EC50 values in the mouse ICOS ELISA assay (mean EC50 values; 0.42 nM±0.075 nM and 0.28±0.037 nM respectively).

STIM003_289 and STIM003_457 gave more robust N=3 EC50 values due to a higher max assay signal and better sigmoidal curve, not observed for STIM001_289 and STIM001_457 in the mouse ICOS ELISA assay.

STIM001_289, STIM003_289 and hybrid control_289 produced similar EC50 values in the human PD-L1 ELISA assay (mean EC50 values; 1.57 nM±0.32 nM, 1.43 nM±0.16 nM and 1.45 nM±0.28 nM respectively).

STIM001_457, STIM003_457 produced similar EC50 values in the mouse PD-L1 ELISA assay (mean EC50 values; 3.84 nM±1.87 nM, 6.83 nM±1.38 nM respectively).

TABLE E3-1

| | hICOS | | | | | |
|---|---|---|---|---|---|---|
| | N = 1 | N = 2 | N = 3 | | | |
| Best-Fit values | EC50 (M) | EC50 (M) | EC50 (M) | Mean (M) | STDEV (M) | SEM (M) |
| STIM003_289 | 4.908E−10 | 5.622E−10 | 8.406E−10 | 6.312E−10 | 1.84804E−10 | 1.06697E−10 |
| STIM001_289 | 9.812E−09 | 9.013E−09 | 2.066E−08 | 1.316E−08 | 6.50816E−09 | 3.75749E−09 |
| STIM003_457 | 5.098E−10 | 3.800E−10 | 4.006E−10 | 4.301E−10 | 6.97426E−11 | 4.02659E−11 |
| STIM001_457 | 1.315E−08 | 1.533E−08 | 9.169E−09 | 1.255E−08 | 3.12347E−09 | 1.80333E−09 |
| STIM003 | 4.880E−10 | 1.037E−09 | 7.270E−10 | 7.507E−10 | 2.75385E−10 | 1.58994E−10 |
| STIM001 | 2.002E−08 | 2.549E−08 | 4.128E−08 | 2.893E−08 | 1.10422E−08 | 6.3752E−09 |

TABLE E3-2

| | mICOS | | | | | |
|---|---|---|---|---|---|---|
| | (N = 1) | (N = 2) | (N = 3) | | | |
| Best-Fit values | EC50 (M) | EC50 (M) | EC50 (M) | Mean (M) | STDEV (M) | SEM (M) |
| STIM003_289 | 3.358E−10 | 4.775E−10 | 4.529E−10 | 4.221E−10 | 7.56844E−11 | 4.36964E−11 |
| STIM001_289 | 2.057* | 2.270E−09 | 2.163E−09 | 2.217E−09 | 7.55591E−11 | 4.36241E−11 |
| STIM003_457 | 3.265E−10 | 2.518E−10 | 2.881E−10 | 2.888E−10 | 3.73468E−11 | 2.15622E−11 |
| STIM001_457 | 2.360E−08 | 5.80E−07* | 437.58* | 2.360E−08 | | |
| STIM003 | 4.16E−10 | 3.86E−10 | 3.38E−10 | 3.799E−10 | 3.94138E−11 | 2.27555E−11 |
| STIM001 | 1.14E−08 | 1.87E−08 | 1.73E−09 | 1.140E−08 | 8.4999E−09 | 4.90742E−09 |

*data excluded due to incomplete curve fit.

TABLE E3-3

| | hPD-L1 | | | | | |
|---|---|---|---|---|---|---|
| | N = 1 | N = 2 | N = 3 | | | |
| Best-Fit values | EC50 (M) | EC50 (M) | EC50 (M) | Mean (M) | STDEV (M) | SEM (M) |
| STIM003_289 | 1.23E−09 | 1.53E−09 | 1.52E−09 | 1.43E−09 | 1.69084E−10 | 9.76206E−11 |
| STIM001_289 | 1.48E−09 | 1.30E−09 | 1.93E−09 | 1.57E−09 | 3.25612E−10 | 1.87992E−10 |
| Hybrid Control_289 | 1.16E−09 | 1.46E−09 | 1.73E−09 | 1.45E−09 | 2.89106E−10 | 1.66915E−10 |
| AbV | 8.34E−10 | 8.18E−10 | 1.13E−09 | 9.28E−10 | 1.76778E−10 | 1.02063E−10 |

TABLE E3-4

| | mPD-L1 | | | | | |
|---|---|---|---|---|---|---|
| | N = 1 | N = 2 | N = 3 | | | |
| Best-values | EC50 (M) | EC50 (M) | EC50 (M) | Mean (M) | STDEV (M) | SEM (M) |
| STIM003_457 | 5.81743E−09 | 8.40577E−09 | 6.28E−09 | 6.836E−09 | 1.380E−09 | 7.965E−10 |
| STIM001_457 | 5.6504E−09 | 3.97431E−09 | 1.91E−09 | 3.845E−09 | 1.874E−09 | 1.082E−09 |
| AbV | 1.38E−09 | 9.99E−10 | 8.66E−10 | 1.080E−09 | 2.644E−10 | 1.527E−10 |

### Example 4 FACS Characterisation of Bispecific Antibody Binding to Cells Expressing ICOS or PD-L1

### CHO Human ICOS and CHO Mouse ICOS Binding Assay (Flow Cytometry)

Ability of the ICOS/PD-L1 mAb² to bind human ICOS and mouse ICOS on the surface of CHO cells was confirmed in this assay. STIM001_289 mAb² and STIM003_289 mAb² were assessed for binding to transfected human ICOS and mouse ICOS CHO cells. Antibody binding to cells was detected with anti-human IgG labelled AlexaFluor 647.

### Method:

CHO-S cells transfected with either human ICOS or mouse ICOS were resuspended in FACS buffer (PBS+1% w/v BSA+0.1% w/v sodium azide) and transferred to a 96-well V-bottom plate (Greiner) at a density of 1×10⁵ cells per well. mAb and mAb² were titrated in FACS buffer, 1 in 3 dilution across 11 points from a starting working concentration of 400 nM. Plates were centrifuged at 300×g for 3 minutes, supernatant discarded and 50 μL mAb or mAb² solution were added to cells and incubated at 4° C. for 1 hour. Cells were washed with 150 μL of PBS and centrifuged at 300×g for 3 minutes, supernatant was discarded and cell pellet resuspended in 150 μL PBS added. This wash step was repeated twice. Bound mAb or mAb² was detected by addition of 50 μL of anti-human 647 (Jackson ImmunoResearch) diluted to 3 ug/ml in FACS buffer. Cells were incubated for 1 hour at 4° C. in the dark. Cells were washed with 150 μL of PBS and centrifuged at 300 g for 3 minutes, supernatant was discarded and cell pellet resuspended in 150 μL PBS added. This wash step was repeated twice. Cells were fixed with 25 μL 4% v/v paraformaldehyde, incubated for 20 minutes at 4° C., cells were pelleted by centrifugation at 300×g and the supernatant discarded. Cells were washed with 150 μL of PBS and centrifuged at 300 g for 3 minutes, supernatant was discarded and cell pellet resuspended in 150 μL PBS added. This wash step was repeated. Pelleted cells were resuspended in 110 μL 1×PBS. AlexaFluor 647 signal intensity (geometric mean) was measured by flow cytometry using a Beckman Coulter CytoFLEX instrument.

### Results

EC50 data for binding to human ICOS are shown in Table E4-1 below and in FIG. 5 (A).

TABLE E4-1

| | hICOS | | | | | |
|---|---|---|---|---|---|---|
| | N = 1 | N = 2 | N = 3 | | | |
| Best-Fit value | EC50 (M) | EC50 (M) | EC50 (M) | Mean (M) | STDEV (M) | SEM (M) |
| STIM003_289 | 5.696E−09 | 2.282E−09 | 6.792E−09 | 4.924E−09 | 2.352E−09 | 1.358E−09 |
| STIM001_289 | 5.529E−09 | 2.982E−09 | 3.134E−09 | 3.882E−09 | 1.429E−09 | 8.249E−10 |
| STIM003 | 7.220E−09 | 4.890E−09 | 4.485E−09 | 5.532E−09 | 1.476E−09 | 8.522E−10 |
| STIM001 | 3.009E−08 | 1.380E−08 | 1.973E−08 | 2.121E−08 | 8.244E−09 | 4.760E−09 |

EC50 data for binding to mouse ICOS are shown in Table E4-2 below and in FIG. 5 (B)

TABLE E4-2

| | mICOS | | | | | |
|---|---|---|---|---|---|---|
| | N = 1 | N = 2 | N = 3 | | | |
| Best-Fit values | EC50 (M) | EC50 (M) | EC50 (M) | Mean (M) | STDEV (M) | SEM (M) |
| STIM003_457 | 8.923E−09 | 1.099E−08 | 1.187E−08 | 1.059E−08 | 1.514E−09 | 8.741E−10 |
| STIM001_457 | 5.138E−09 | 3.530E−09 | 6.974E−09 | 5.214E−09 | 1.724E−09 | 9.951E−10 |
| STIM003 | 6.803E−09 | 2.195E−09 | 7.275E−09 | 5.424E−09 | 2.807E−09 | 1.620E−09 |
| STIM001 | 2.567E−08 | 5.063E−09 | 2.008E−08 | 1.694E−08 | 1.066E−08 | 6.152E−09 |

### CHO Human PD-L1 Binding Assay (Flow Cytometry)

Ability of the ICOS/PD-L1 mAb² to bind human PD-L1 expressed on the surface of CHO cells was confirmed in this assay. Binding of the bispecific antibody to PD-L1 could be detected using labelled ICOS recombinant protein, confirming the ability of the bispecific antibodies to bind both PD-L1 and ICOS.

STIM001_289 and STIM003_289 and one isotype control (IgG1_289) were assessed for human PD-L1 binding using FACS. These were characterised using anti-human IgG and human ICOS labelled AlexaFluor 647 detection.

### Method:

CHO-S cells untransfected (referred to as WT) or transfected with the cDNA coding for human PD-L1 were diluted in FACS buffer (PBS+1% w/v BSA+0.1% w/v sodium azide) and were distributed to a 96-well V-bottom plate (Greiner) at a density of 5×10⁴ cells per well. Antibody and mAb² titrations were prepared from 198 nM working concentration as a 1/3 dilution series in FACS buffer. Plates were centrifuged at 300×g for 3 minutes to supernatant aspirated. 50 μL antibody or mAb² solution were added to cells and incubated at 4° C. for 1 hour. Cells were washed with 150 μL of PBS and centrifuged at 300 g for 3 minutes. Supernatant was aspirated and 150 μL PBS added. This wash step was repeated. Presence of bound antibody or mAb² was detected by addition of 50 μL of Anti-Human PE (Jackson ImmunoResearch) diluted 1/500 in FACS buffer or human ICOS labelled AlexaFluor 647 diluted to 225 nM to each well. Cells were incubated for 1 hour at 4° C. in the dark. Cells were washed as previously described. To fix cells, 100 μL 4% v/v paraformaldehyde was added and cells incubated for 20 minutes at 4° C., cells were pelleted by centrifugation at 300×g and the plates resuspended in 100 μL FACS buffer. AlexaFluor 647 and PE (R-Phycoerythrin) signal intensity (geometric mean) was measured by flow cytometry using a Beckman Coulter CytoFLEX instrument.

### Results

Bispecific antibodies and isotype control produced similar EC50 values to each other in the anti-human IgG detection system (0.64 nM, 0.64 nM and 0.55 nM respectively)-FIG. 6 (A). Bispecific antibodies produced similar EC50 values to each other in the human ICOS labelled AlexaFluor 647 system (0.48 nM and 0.58 nM respectively)-FIG. 6 (B). As expected, the isotype control antibody did not bind ICOS.

Also as expected, monospecific antibodies STIM001, STIM003 and control IgG1 did not show binding to human PD-L1 with either anti-human IgG detection or human ICOS labelled AlexaFluor 647.

### CHO Mouse PD-L1 Binding Assay (Flow Cytometry)

Ability of the ICOS/PD-L1 mAb² to bind mouse PD-L1 on the surface of CHO cells was confirmed in this assay. Binding of the bispecific antibody to PD-L1 could be detected using ICOS, confirming the ability of the bispecific antibodies to bind both PD-L1 and ICOS.

STIM001_457 and STIM003_457 and one isotype control (IgG1_438) were assessed for human PD-L1 binding using FACS. These were characterised using anti-human IgG and human ICOS labelled AlexaFluor 647 detection.

### Method:

CHO-S cells untransfected (referred to as WT) or transfected with mPD-L1 were diluted in FACS buffer (PBS+1% w/v BSA+0.1% w/v sodium azide) and were distributed to a 96-well V-bottom plate (Greiner) at a density of 5×10⁴ cells per well. Monospecific mAb and bispecific mAb² titrations were prepared from 22 nM working concentration as a 1/3 dilution series in FACS buffer. Plates were centrifuged at 300×g for 3 minutes to supernatant aspirated. 50 μL antibody or mAb² solution were added to cells and incubated at 4° C. for 1 hour. Cells were washed with 150 μL of PBS and centrifuged at 300 g for 3 minutes. Supernatant was aspirated and 150 μL PBS added. This wash step was repeated. Presence of bound mAb or mAb² was detected by addition of 50 μL of Anti-Human IgG PE (Jackson ImmunoResearch) diluted 1/500 in FACS buffer or human ICOS labelled AlexaFluor 647 diluted to 25 nM to each well. Cells were incubated for 1 hour at 4° C. in the dark. Cells were washed as previously described. To fix cells, 100 μL 4% v/v paraformaldehyde was added and cells incubated for 20 minutes at 4° C., cells were pelleted by centrifugation at 300×g and the plates resuspended in 100 μL FACS buffer. AlexaFluor 647 and PE (R-Phycoerythrin) signal intensity (geometric mean) was measured by flow cytometry using a Beckman Coulter CytoFLEX instrument.

### Results

Bispecific antibodies and isotype control produced similar EC50 values to each other in the anti-human IgG detection system (0.89±0.64 nM, 0.47±0.23 nM and 0.59±0.20 nM respectively)—FIG. 7 (A)—and in the human ICOS labelled AlexaFluor 647 system (1.29±1.26 nM and 0.81±0.56 nM respectively)—FIG. 7 (B). The isotype control did show binding.

Monospecific antibodies STIM001, STIM003 and IgG1 did not show binding to human PD-L1 with either anti-human IgG detection or human ICOS labelled AlexaFluor 647.

### Example 5a Biolayer Interferometry Determination of mAb² Binding to Fcγ Receptors

### Materials

Sensors coated with a commercial anti-human FAb-CH1 ligand (Pall ForteBio, cat No 18-5125) were hydrated for 10 min in running buffer (1×HBS-EP+Buffer: Technova, cat. No. H8022).

The mAb²_289 constructs STIM001_289 and STIM003_289 were diluted to 45 μg/mL in running buffer.

The recombinant human FcγRI (1257-FC-050, R&D Systems), mouse FcγRI (2074-FC-050, R&D Systems) and mouse FcγRIV (1974-CD-050, R&D Systems) were diluted to 1 UM in running buffer. The recombinant human FcγRIIIa (4325-FC-050, R&D Systems) and mouse FcγRIII (1960-FC-050, R&D Systems) were diluted to 2 μM in running buffer. Finally, the recombinant human FcγRIIa (1330-CD-050, R&D Systems), human FcγRIIb/c (1875-CD-050, R&D Systems) and mouse FcγRIIb (1460-CD-050, R&D Systems) were diluted to 3 μM in running buffer.

The human recombinant extracellular domain PD-L1 protein was diluted to 1 μM in running buffer.

### Method

The anti-human FAb-CH1 sensors were regenerated with 100 mM PO₄ then equilibrated in running buffer. STIM001_289 and STIM003_289 were captured at 45 μg/mL on the sensors and then loaded with 1 μM of human PD-L1. Finally, the sensors were dipped into the Fcγ receptor solution. The sensors were then regenerated and equilibrated again, and the same protocol was repeated for each Fcγ receptor. The assay was carried out at 25° C.

### Results

| Fc gamma Receptor | STIM001_289 | STIM003_289 |
|---|---|---|
| Human FcγR I | Binding | Binding |
| Human FcγR IIa | Binding | Binding |
| Human FcγR IIb/c | Binding | Binding |
| Human FcγR IIIa | Binding | Binding |
| Mouse FcγR I | Binding | Binding |
| Mouse FcγR IIb | Binding | Binding |
| Mouse FcγR III | Binding | Binding |
| Mouse FcγR IV | Binding | Binding |

In this assay, both mAb²_289 constructs (STIM001_289 and STIM003_289) demonstrated expected binding to all the individual Fcγ receptors.

### Example 5b Engagement of Fc Receptor on ADCC Effector Cells by Fc Region of Bispecific Antibody to ICOS and PD-L1

This assay determines ability of the Fc region of mAb² to engage FcγRIIIa on effector cells.

### Method

Immediately prior to the assay CHO (target) cells expressing human ICOS or mouse ICOS were centrifuged and resuspended in RPMI 1640 (Promega)+4% Low IgG serum (Promega) and plated at 50000 cells/well (25 μl/well) in 96-well white bottom TC-treated plates (Costar).

All antibodies were serially diluted 1 in 3 over 9 points, in RPMI 1640+4% Low IgG serum. For assays with human ICOS expressing target cells, the antibodies were diluted from starting working concentration of 10 nM and for mouse ICOS expressing target cells the mAb² and mAb were diluted from a starting working concentration of 20 nM and 35 nM, respectively. Diluted antibodies (25 μl/well) were added to the target cells and left to incubate for 0.5 hrs at room temperature. Thawed Jurkat NFAT luciferase v-variant effector cells (Promega) were resuspended in RPMI 1640+4% low IgG serum and added to the target cell/antibody mixture at 10000 cells/well (25 μl/well). After overnight incubation at 37° C., 5% CO2, luciferase activity was measured by adding luminogenic BioGlo substrate at 75 μl/well (Promega) directly to the wells, plates incubated for 10 min in the dark and read on an Envision (Perkin Elmer) plate reader.

Relative light unit (RLU) values from the raw data (Envision reads) were first normalised to ‘fold of induction’ using the following equation.

$$Fold of induction = \frac{RLU ( induced ) - RLU ( background )}{RLU ( background )}$$

Mean and standard deviation ‘fold of induction’ values for each antibody concentration were plotted and curves were fitted using the GraphPad Prism 4-parameter log-logistic curve. The average ‘fold of induction’ values for each experiment were then used to plot the inter-experimental values (+/−SD) from all three experiments.

### Results

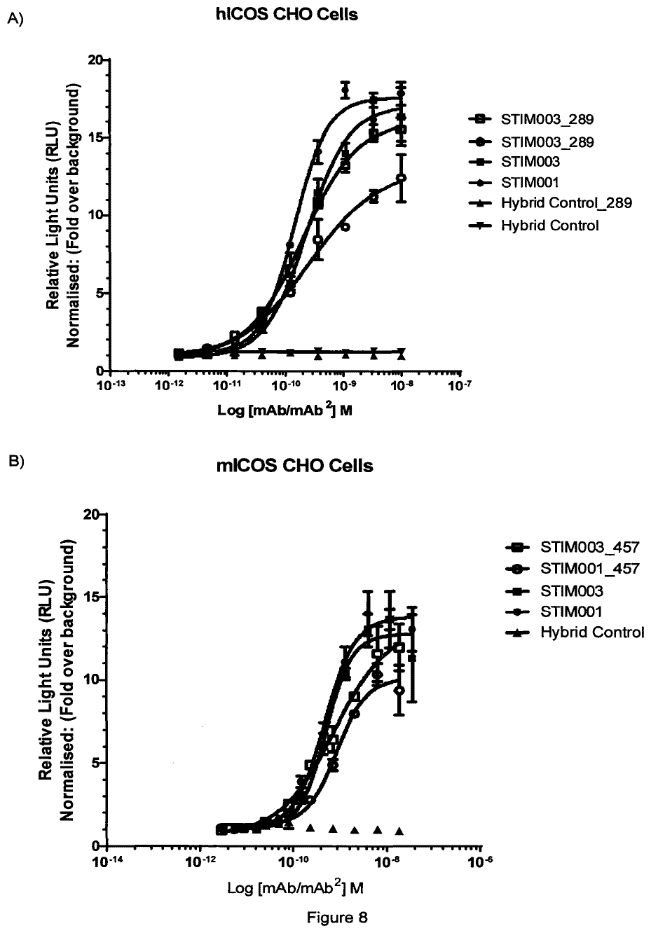

Data are summarised in Table E5-1 and in FIGS. 8A and B. STIM003_289 and STIM001_289 produced similar EC50 values in the human and mouse ICOS ADCC assay.

TABLE E5-1

| | hICOS | | | | | |
|---|---|---|---|---|---|---|
| | N = 1 | N = 2 | N = 3 | | | |
| Best-Fit value | EC50 (M) | EC50 (M) | EC50 (M) | Mean (M) | STDEV (M) | SEM (M) |
| STIM003_289 | 2.309E−10 | 4.009E−10 | 2.069E−10 | 2.796E−10 | 1.058E−10 | 6.108E−11 |
| STIM001_289 | 1.419E−09 | 4.019E−10 | 2.493E−10 | 6.900E−10 | 6.358E−10 | 3.671E−10 |
| STIM003 | 3.258E−10 | 4.769E−10 | 2.397E−10 | 3.474E−10 | 1.201E−10 | 6.933E−11 |
| STIM001 | 4.559E−10 | 2.467E−10 | 1.467E−10 | 2.831E−10 | 1.578E−10 | 9.112E−11 |

| | mICOS | | | | | |
|---|---|---|---|---|---|---|
| | N = 1 | N = 2 | N = 3 | | | |
| Best-Fit values | EC50 (M) | EC50 (M) | EC50 (M) | Mean (M) | STDEV (M) | SEM (M) |
| STIM003_457 | 7.702E−10 | 1.353E−09 | 1.894E−09 | 1.339E−09 | 5.621E−10 | 3.245E−10 |
| STIM001_457 | 8.627E−10 | 4.888E−09* | 1.077E−08* | 8.627E−10 | | |
| STIM003 | 5.079E−10 | 7.544E−10 | 7.025E−10 | 6.549E−10 | 1.299E−10 | 7.502E−11 |
| STIM001 | 4.790E−10 | 5.337E−10 | 5.786E−10 | 5.304E−10 | 4.988E−11 | 2.880E−11 |

*EC50 values excluded due to incomplete curve fit.

### Example 5c ADCC Assay Using Peripheral Blood Mononuclear Cells (PBMC)

The potential to kill via ADCC (“antibody-dependent cell-mediated cytotoxicity”) of STIM001_289 and STIM003_289 was compared with that of STIM001 and STIM003 in the Delfia BATDA cytotoxicity assay (Perkin Elmer) using human primary NK cells as effector and ICOS-transfected CCRF-CEM cells as target cells. This method is based on loading target cells with an acetoxymethyl ester of fluorescence enhancing ligand (BATDA) which quickly penetrates the cell membrane. Within the cell the ester bonds are hydrolysed to form a hydrophilic ligand (TDA) which no longer passes the membrane. After cytolysis, the ligand is released and can be detected by addition of Europium which forms with the TDA a highly fluorescent and stable chelate (EuTDA). The measured signal correlates directly with the amount of lysed cells.

Isolation of Mononuclear Cells from Human Peripheral Blood:

Leukocyte cones were collected from healthy donors and their content was diluted up to 50 ml with phosphate buffered saline (PBS, from Gibco) and layered into 2 centrifuge tubes on top of 15 mL Ficoll-Paque (from GE Healthcare). PBMC were separated by density gradient centrifugation (400 g for 40 min without brake), transfer in a clean centrifuge tube and then wash with 50 mL PBS, twice by centrifuging at 300 g for 5 min and twice by centrifuging at 200g for 5 min. PBMC were then resuspended in R10 media (RPMI+10% heat-inactivated Fetal Bovine Serum, both from Gibco) and their cell count and viability assess with EVE™ Automated Cell Counter (from NanoEnTek).

### ADCC Assay Methods:

Labelling of target cells was performed according to manufacturer's instruction. Briefly, CCRF-CEM cells were resuspended at 1×10⁶/mL in assay media (RPMI+10% ultra-low IgG FBS, from Gibco) and loaded with 5 μl/mL of Delfia BATDA reagent (Perkin Elmer) for 30 min at 37° C. Cells were then washed 3 times with 50 mL PBS (300 g for 5 min) and resuspended at 8×10⁵/ml (3×) in assay media.

STIM001_289, STIM003_289, STIM001, STIM003 and their isotype controls, IgG1_289 and IgG1 were serially diluted 1:4 in assay media to give final 3× antibody concentrations ranging from 30 nM to 0.12 pM (10-point curve).

NK cells were negatively isolated from PBMC using the EasySep Human NK Cell Isolation Kit (from Stemcell Technologies) and resuspended at 4×10⁶/ml (3×) in assay media.

BATDA-loaded CCRF-CEM and primary NK cells were co-cultured for 4-hours at 37° C. and 5% CO2 at a 5:1 Effector:Target ratio in assay media in the presence of the antibodies under investigation (from 10 nM to 0.04 PM final concentration). Wells containing CCRF-CEM cells only or CCRF-CEM+Delfia lysis buffer (Perkin Elmer) were used to determine spontaneous and 100% BATDA release, respectively. Cell-free supernatants were then transferred into a DELFIA Microtitration Plates and incubated for 15 min at Room Temperature with the Delfia Europium solution (Perkin Elmer). Fluorescent signal at 615 nM was then quantified with Envision Multilabel Reader (PerkinElmer).

Specific dye release induced by the Abs was calculated as: [(Experimental release−Spontaneous release)/(Maximum release−Spontaneous release)]*100.

This experiment was repeated with NK-cells from 2 independent donors and 3 technical replicates were included for each assay condition.

### Results

The ability of STIM001_289 and STIM003_289 bispecific antibodies to induce ADCC was assessed using primary NK cells from 3 independent donors, as effector cells and ICOS-transfected CCRF-CEM as target cells. STIM001 and STIM003 and the relevant isotype controls (IgG1 and IgG1_289) were run in the same experiments. Target cells were loaded with BADTA dye and incubated for 4 hrs either alone (spontaneous release), with lysis buffer (100% release) or with NK cells plus increasing concentration of antibody. Dye release correlates directly with the number of lysed cells. Data are showed as % of specific dye release and plotted against the log of antibody concentrations (FIG. 9). In all donors, STIM001_289, STIM003_289, STIM001 and STIM003 increased the % of specific dye release in a concentration dependent manner. Non-linear regression curves (variable slope, 4-parameter) were extrapolated from the data obtained. These data demonstrate that the bispecific constructs have the ability to kill ICOS positive cells in a primary NK dependent ADCC assay. The EC50s of the bispecific antibodies in the ADCC assay were comparable to those of STIM001 and STIM003 monoclonal IgG1 antibodies. See Table E5-2 and FIG. 9 A-C.

TABLE E5-2
EC50 (pM) of STIM001_289 and STIM003_289 antibodies in the
ADCC assay using ICOS-transfected CCRF-CEM cells and freshly
isolated NK cells as effector cells (non-linear fit, 4-parameters, n = 2).

| EC50 (pM) | Donor 326 | Donor 334 | Donor 341 | Median | SD |
|---|---|---|---|---|---|
| STIM001 | 42.6 | 20.5 | 7.6 | 23.6 | 17.7 |
| STIM001_289 | 30.9 | 4.1 | ~10.3 | 17.5 | 19.0 |
| STIM003 | 21.2 | 3.7 | 6.7 | 10.5 | 9.4 |
| STIM003_289 | 11.1 | 18.3 | 6.6 | 12.0 | 5.9 |

### Example 6 Ability of ICOS/PD-L1 Bispecific Antibody to Neutralise ICOS Binding to ICOS Ligand

Anti-ICOS antibodies in monoclonal and mAb² format were assessed for ICOS ligand (B7-H2) neutralisation using HTRF. These antibodies are capable of neutralising both human and mouse ICOS B7-H2 ligand and were assessed in both Human ICOS Receptor/Human Ligand and Mouse ICOS Receptor/Mouse Ligand HTRF based Neutralisation assays.

### Method

Antibodies were diluted in assay buffer (0.53M Potassium Fluoride (KF), 0.1% Bovine Serum Albumin (BSA) in 1×PBS) from a starting working concentration of either 1 μM or 0.4 UM and serially diluted 1 in 3 over 11 points. 5 μl of titrated antibody were added to 384w solid white assay plate (Greiner Bio-One). Positive and negative control wells received 5 μl of assay buffer only.

5 μl of human ICOS-mFc (Chimerigen) at 20 nM (5 nM final) or 5 μl of mouse ICOS-mFc (Chimerigen) at 4 nM (1 nM final) were added to relevant assay wells. Plate was incubated for 1 hour (hr) at room temperature (RT).

After incubation, 5 μl of ICOS ligand, (B7-H2, R&D Systems) conjugated to Alexa 647 (Innova Bioscience) was diluted to either 14.08 nM (3.52 nM final) for human B7-H2 or 36.08 nM (9.02 nM final) for mouse B7-H2 and added to all wells of assay plate except negative control wells which instead received 5 μl of assay buffer.

Finally, 5 μl of 4.32 nM anti-mouse IgG donor mAb (Southern Biotech) labelled with europium cryptate (Cis Bio), was added to each well and the assay was left in the dark at RT to incubate for a further 2 hours. After incubation, assay was read on Envision plate reader (Perkin Elmer) using a standard HTRF protocol. 620 nm and 665 nm channel values were exported to Microsoft Excel and % Delta-F and % Neutralisation calculations performed. Titration curves and IC50 values [M] were plotted using Graphpad (Prism). IC50 values were calculated by first transforming the data using equation X=Log(X). The transformed data was then fitted using nonlinear regression, using fitting algorithm, log (inhibitor) vs. response-variable slope (four parameters).

$$\begin{matrix}\% Delta - F Calculation & \\ 665 / 620 nm ratio for ratio metric data reduction . & \end{matrix}$$

$$\% Delta F = \frac{( 665 / 620 nm Well Signal Ratio - Signal Negative Control )}{( Signal Negative Control )} {}^{*} 100$$

$$Signal Negative control = average of minimum signal ratio .$$

$$\begin{matrix}\% Neutralisation & \\ \% Max ( neutralisation ) = \frac{( \% Delta - F of sample well - Negative Control )}{( Positive Control - Negative Control )} {}^{*} 100 & \end{matrix}$$

### Results

In the human ICOS ligand neutralisation system, STIM003_289 and STIM003_457 produced similar IC50 values to STIM003 (mean IC50 values, 0.81±0.28 nM, 0.56±0.18 nM and 0.53±0.15 nM respectively).

In the human ICOS ligand neutralisation system, STIM001_289 and STIM001_457 produced similar IC50 values to STIM001 (mean IC50 values, 2.0±1.7 nM, 1.6±6.9 nM and 1.5±0.75 nM respectively).

In the mouse ICOS ligand neutralisation system, STIM003_289 and STIM003_457 produced similar IC50 values to STIM003 (mean IC50 values, 0.14±0.037 nM, 0.11±0.027 nM and 0.12±0.027 nM and respectively).

In the mouse ICOS ligand neutralisation system, STIM001_289 and STIM001_457 produced similar IC50 values to STIM001 (mean IC50 values, 4.8±1.7 nM, 5.16±1.5 nM and 8.7±6.6 nM and respectively).

In the human ICOS ligand neutralisation system, STIM003 produced similar IC50 values to STIM001 (mean IC50 values, 0.53±0.15 nM, 1.5±0.75 nM respectively).

In the mouse ICOS ligand neutralisation system, STIM003 produced more potent IC50 values to STIM001 (mean IC50 values, 0.12±0.027 nM and 8.7±0.66 nM respectively).

Data are summarised in Table E6-1 and Table E6-2 and FIGS. 10A and B.

TABLE E6-1

| | IC50 Human ICOS Receptor/ | | | | |
|---|---|---|---|---|---|
| | Human B7-H2 | | | Average | SD |
| Ab | n1 (nM) | n2 (nM) | n3 (nM) | IC50 (nM) | (nM) |
| STIM003_289 | 0.58 | 0.73 | 1.11 | 0.81 | 0.27 |
| STIM001_289 | 1.00 | 0.94 | 3.95 | 1.96 | 1.72 |
| STIM003_457 | 0.44 | 0.47 | 0.77 | 0.56 | 0.18 |
| STIM001_457 | 1.10 | 1.32 | 2.38 | 1.60 | 0.68 |
| STIM003 | 0.43 | 0.45 | 0.71 | 0.53 | 0.16 |
| STIM0001 | 0.96 | 1.12 | 2.33 | 1.47 | 0.75 |

TABLE E6-2

| | IC50 Mouse ICOS Receptor/Mouse B7-H2 | | | | |
|---|---|---|---|---|---|
| | | | | Average | SD |
| Patent Ab | n1 (nM) | n2 (nM) | n3 (nM) | IC50 (nM) | (nM) |
| STIM003_289 | 0.10 | 0.17 | 0.13 | 0.13 | 0.04 |
| STIM001_289 | 3.25 | 6.53 | 4.57 | 4.78 | 1.65 |
| STIM003_457 | 0.087 | 0.13 | 0.10 | 0.11 | 0.02 |
| STIM001_457 | 3.39 | 5.49 | 6.32 | 5.07 | 1.51 |
| STIM003 | 0.87 | 0.14 | 0.11 | 0.37 | 0.43 |
| STIM001 | 7.98 | 8.91 | 9.25 | 8.71 | 0.66 |

### Example 7 Ability of ICOS/PD-L1 Bispecific Antibody to Neutralise PD-L1 Binding to PD1 or CD80

### Neutralisation of Human PD-L1 Binding

Bispecific mAb² antibodies STIM001_289 and STIM003_289, two anti-PD-L1 antibodies AbW and AbV, and one isotype control (IgG1_289), were assessed for ability to neutralise human PD-L1 binding to its receptors human PD1 and human CD80 on CHO cells, using flow cytometry (FACS). Ability of the mAb² to neutralise binding of human PD-L1 to its receptors was confirmed in this assay.

### Method

CHO-S cells untransfected (referred to as WT) or transfected with human PD-L1 were diluted in FACS buffer (PBS+1% w/v BSA+0.1% w/v sodium azide) and were distributed to a 96-well V-bottom plate (Greiner) at a density of 5×10⁴ cells per well. Biotinylated human CD80-Fc (R&D Systems) or PD-1-Fc were prepared as a standard curve titration from 1 μM final assay concentration (FAC), 1/3 dilution series in FACS buffer. Antibody and mAb² titrations were prepared from 396 nM working concentration, 198 nM FAC, as a 1/3 dilution series in FACS buffer. Biotinylated PD-1 or CD80 were diluted in FACS buffer to 80 nM working concentration, 40 nM FAC. Plates were centrifuged at 300×g for 3 minutes to supernatant aspirated. 25 μL receptor and 25 μL mAb² solution (or 50 μL of receptor standard curve titration) were added to cells and incubated at 4° C. for 1 hour. Cells were washed with 150 μL of PBS and centrifuged at 300 g for 3 minutes. Supernatant was aspirated and 150 μL PBS added. This wash step was repeated. Presence of bound CD80 or PD-1 was detected by addition of 50 μL of streptavidin-AlexaFluor 647 (Jackson ImmunoResearch) diluted 1/500 in FACS buffer to each well. Cells were incubated 1 hr at 4° C. in the dark. Cells were washed as previously described. To fix cells, 100 μL 4% v/v paraformaldehyde was added and cells incubated for 20 minutes at 4° C., cells were pelleted by centrifugation at 300×g and the plates resuspended in 100 μL FACS buffer. AlexaFluor 647 signal intensity (geometric mean) was measured by flow cytometry using a Beckman Coulter CytoFLEX.

$$Percentage of receptor binding ( flow cytometry ) : \qquad Equation X$$

$$Based on geometric mean fluorescence$$

$$\% of specific binding = \frac{sample value - non - specific binding}{total binding - non - specific binding} \times 100$$

$$Total binding = biotinylated PD - 1 or CD 80 only ( Isotype antibody at 198 nM )$$

$$Non - specific binding = {mAb}^{2} at concentration of 198 nM$$

### Results

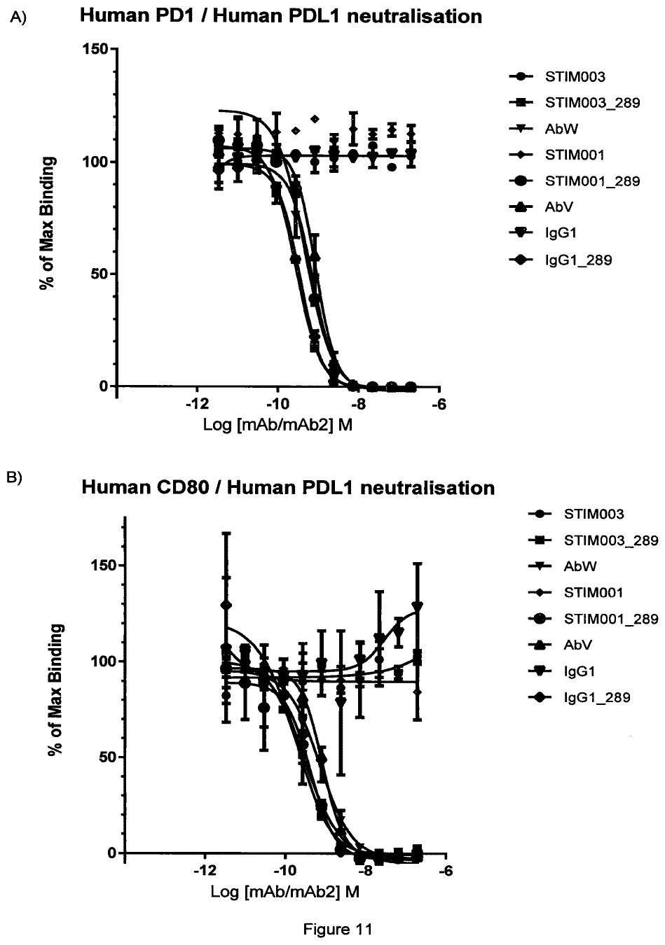

Data are shown in Tables E7-1 and E7-2 below and in FIGS. 11A and B.

TABLE E7-1
IC50 (nM) values for human PD1/PD-L1 neutralisation, detected with
streptavidin-AlexaFluor 647. Not calculated: Ab was used in assay
with no complete IC50 curve being calculable. See FIG. 11 A.

| | 1050 Human PD1/PD-L1 Neutralisation |
|---|---|
| | (nM) |
| STIM003 | Not Calculated |
| STIM003_289 | 0.28 |
| AbW | 0.70 |
| STIM001 | Not Calculated |
| STIM001_289 | 0.47 |
| AbV | 0.86 |
| IgG1 | Not Calculated |
| IgG1_289 | 0.34 |

TABLE E7-2
IC50 (nM) values for human CD80/PD-L1 neutralisation, detected
with streptavidin-AlexaFluor 647. Not calculated: Ab was used in
assay with no complete IC50 curve being calculable. See FIG. 11 B.

| | IC50 Human CD80/PD-L1 |
|---|---|
| | Neutralisation (nM) |
| STIM003 | Not Calculated |
| STIM003_289 | 0.26 |
| AbW | 0.74 |
| STIM001 | Not Calculated |
| STIM001_289 | 0.43 |
| AbV | 0.85 |
| IgG1 | Not Calculated |
| IgG1_289 | 0.21 |

### Neutralisation of Mouse PD-L1 Binding

Ability of the ICOS/PD-L1 bispecific antibody to neutralise binding of mouse PD-L1 to its receptors was confirmed in this assay.

### Method

CHO-S cells untransfected (referred to as WT) or transfected with mouse PD-L1 were diluted in FACS buffer (PBS+1% w/v BSA+0.1% w/v sodium azide) and were distributed to a 96-well V-bottom plate (Greiner) at a density of 5×104 cells per well. Biotinylated mouse PD-1-Fc (R&D Systems) or CD80-Fc (R&D Systems) were prepared as a standard curve titration from 1 μM final assay concentration (FAC), 1/3 dilution series in FACS buffer. Antibody and mAb² titrations were prepared from 44 nM working concentration, 22 nM FAC, as a 1/3 dilution series in FACS buffer. Biotinylated PD-1 or CD80 were diluted in FACS buffer to 80 nM working concentration, 40 nM FAC. Plates were centrifuged at 300×g for 3 minutes to supernatant aspirated. 25 μL receptor and 25 μL mAb² solution (or 50 μL of receptor standard curve titration) were added to cells and incubated at 4° C. for 1 hour. Cells were washed with 150 μL of PBS and centrifuged at 300 g for 3 minutes. Supernatant was aspirated and 150 μL PBS added. This wash step was repeated. Presence of bound CD80 or PD-1 was detected by addition of 50 μL of streptavidin-AlexaFluor 647 (Jackson ImmunoResearch) diluted 1/500 in FACS buffer to each well. Cells were incubated 1 hr at 4° C. in the dark. Cells were washed as previously described. To fix cells, 100 μL 4% v/v paraformaldehyde was added and cells incubated for 20 minutes at 4° C., cells were pelleted by centrifugation at 300×g and the plates resuspended in 100 μL FACS buffer. AlexaFluor 647 signal intensity (geometric mean) was measured by flow cytometry using a Beckman Coulter CytoFLEX.

$$Percentage of receptor binding ( flow cytometry ) \qquad Equation X$$

$$Based on geometric mean fluorescence$$

$$\% of specific binding = \frac{sample value - non - specific binding}{total binding - non - specific binding} \times 100$$

$$Total binding = biotinylated PD - 1 or CD 80 only ( Isotype antibody at 22 nM FAC )$$

$$Non - specific binding = {mAb}^{2} at concentration of 22 nM FAC$$

### Results

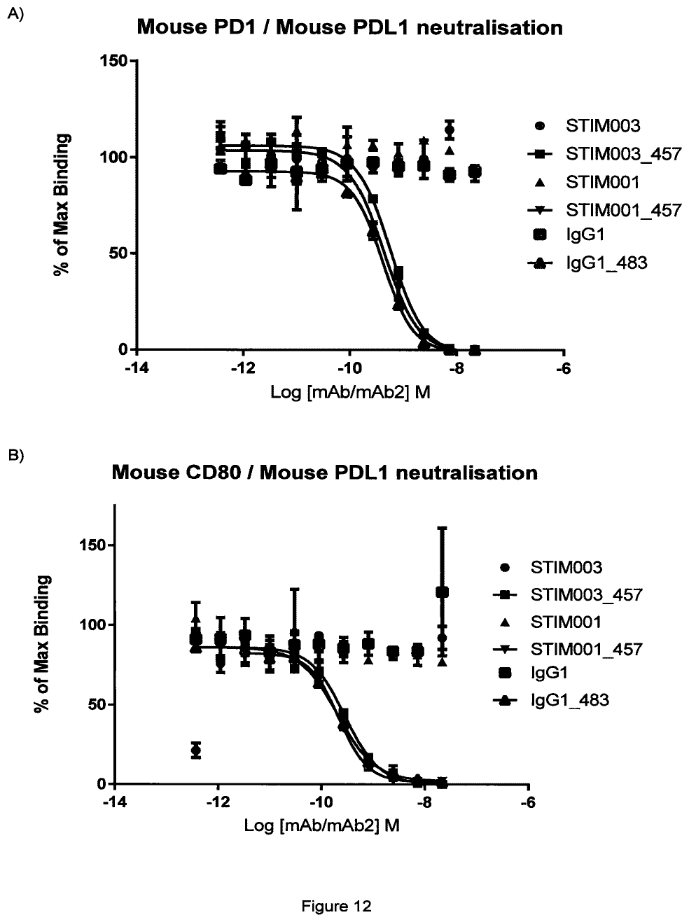

The bispecific mAb²s and an isotype control antibody IgG1_438 were assessed for ability to neutralise binding of mouse PD-L1 to its receptors mouse PD1 and mouse CD80 using FACS. Results are shown in Table E7-3, Table E7-4 and FIG. 12.

STIM001_457, STIM003_457 and IgG1_438 produced similar IC50s (0.35±0.09 nM, 0.40±0.13 nM, and 0.34±0.05 nM respectively) using the PD-L1 and PD1 neutralising system. These mAb²s also neutralised PD-L1 binding to CD80 (IC50, 0.9±0.03 nM, 0.29±0.0002 nM and 0.27±0.06 nM respectively).

Monoclonal antibodies STIM001, STIM003 and IgG1 control did not neutralise mouse PD-L1 binding to mouse PD1 or mouse CD80.

### Example 8 Effect of ICOS/PD-L1 Bispecific Antibody on T Cells in ICOS-Dependent Activation Assay

The agonistic potentials of STIM001_289 and STIM003_289 bispecific antibodies in this assay were compared with those of STIM001 and STIM003 monoclonal antibodies in a human primary T-cell activation assay where anti-CD3 and anti-CD28 antibodies were added concurrently to induce ICOS expression on effector T-cells. Effect of the ICOS co-stimulation on the level of IFN-γ produced by these activated T-cells were assessed using ELISA at 72 hrs post-activation. This assay is used to confirm activity of the ICOS-binding portion of the bispecific antibody. Retention of ability to induce ICOS-mediated T cell activation was confirmed for anti-ICOS antibodies STIM001 and STIM003 in the ICOS/PD-L1 mAb² bispecific format in which the Fcab region binds human PD-L1.

### Methods

PBMC were isolated from human peripheral blood as described in Example 5c and stored in nitrogen for further utilisation.

STIM001_289, STIM003_289, STIM001, STIM003 and their isotype controls, IgG1_289 and IgG1 were serially diluted 1:3 in PBS to give final antibody concentrations ranging from 10 μM to 40 nM (6-point curve). 100 μL of diluted antibodies were coated in duplicate into a 96-well, high-binding, flat-bottom plate (Corning EIA/RIA plate) overnight at 4° C. Plate was then washed with PBS. In some experiments an anti-PD-L1 antibody, AbV, was added at the same concentrations.

T-cells were negatively isolated from frozen PBMC using the EasySep Human T Cell Isolation Kit (from Stemcell Technologies) and resuspended at 2×10⁶/ml in R10 media supplemented with 40 μl/ml of Dynabeads Human T-Activator CD3/CD28 (from Life Technologies).

T-cell suspensions were added to antibody-coated plates to give a final cell concentration of 1×10⁶ cells/ml and cultured for 72 hrs at 37° C. and 5% CO2. Cell free supernatants were then collected and kept at minus 20° C. until analysis of secreted IFN-γ by ELISA with the R&D Systems™ Human IFNγ Duoset® ELISA, using DELFIA® Eu-N1 Streptavidin detection.

This experiment was repeated on T-cells isolated from 4 independent donors and 2 technical replicates were included for each assay condition.

### Results

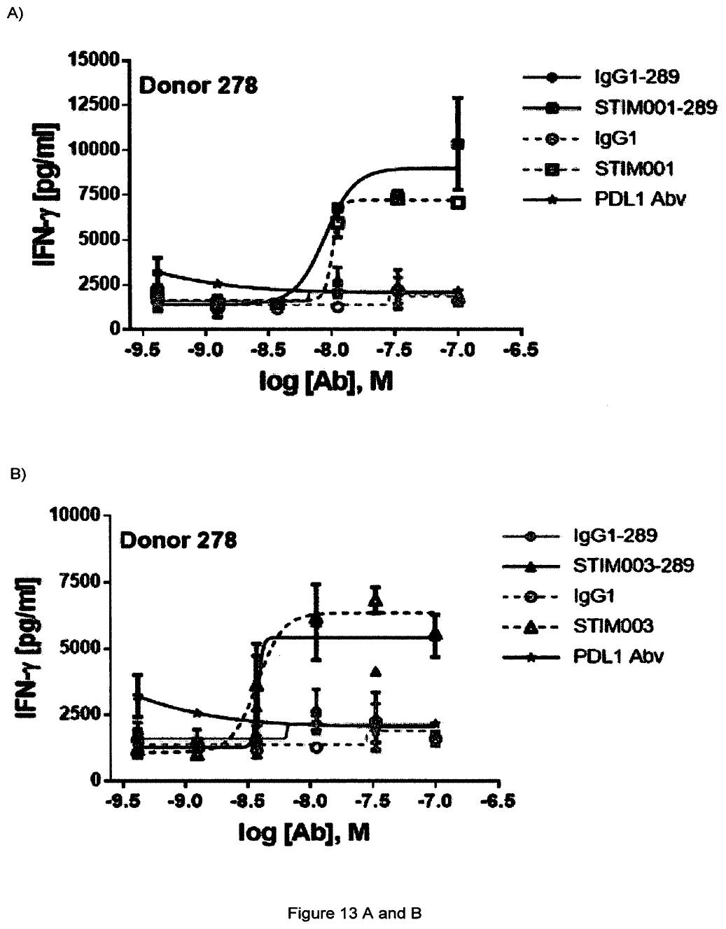

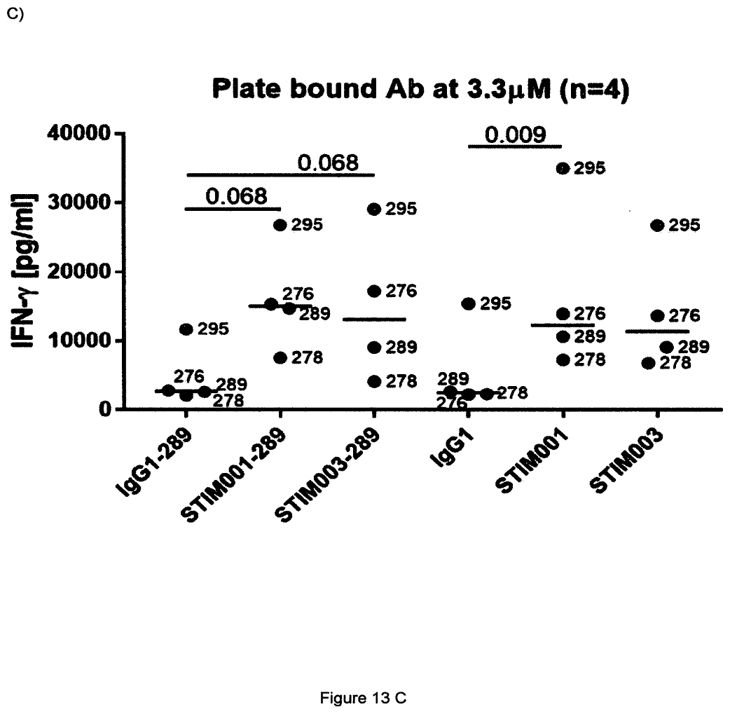

The levels of IFN-γ induced by STIM001_289, STIM003_289, STIM001, STIM003 and their isotype controls (IgG1_289 and IgG1) were measured in assay replicates and plotted as mean value±Standard Deviation (SD) against the log of antibody concentrations, as showed for one donor (FIGS. 13, A and B). All antibodies increased the levels of IFN-γ in a concentration dependent manner. In some experiments, anti-PD-L1 AbV was added as the same concentration and did not modify the levels of IFN-γ. Non-linear regression curves (variable slope, 4-parameter) were extrapolated from data obtained with 4 independent donors (EC50 values in Table E8). Both STIM001_289 and STIM003_289 increased the IFN-γ levels to the same extent as STIM001 and STIM003 respectively. Both EC50 values (Table E8) and levels of IFN-γ at the plateau (3.3 μM, FIG. 13 C) were comparable for STIM001 vs STIM001_289 and for STIM003 vs STIM003_289 (median values). These data demonstrate that the ICOS binding sites of the anti-ICOS monoclonal antibodies have conserved their ICOS agonistic effect on human primary T-cells when included in the ICOS/PD-L1 bispecific antibodies.

TABLE E8
Concentration response curves of IFN-γ following antibody
treatments (non-linear fit, 4-parameters, n = 4).

| | T-cells from donors | | | | | |
|---|---|---|---|---|---|---|
| EC50 (nM) | 278 | 276 | 289 | 295 | Median | SD |
| STIM001 | ~10.2 | ~9.9 | ~11.2 | 14.5 | 10.7 | 2.11 |
| STIM001-289 | 8.9 | ~10 | 7.1 | 11.9 | 9.45 | 2.01 |
| STIM003 | 3.8 | 7.1 | ~4 | ~11.6 | 5.55 | 3.64 |
| STIM003-289 | ~3.8 | ~6.0 | 10.2 | 12.7 | 8.1 | 4.02 |

~value corresponding to the best fit found by GraphPad Prism

### Example 9 Effect of ICOS/PD-L1 Bispecific Antibody on T Cell Activation in Monocyte Co-Culture Primary Cell Assay

This assay can be used to assess ability of the PD-L1 binding site of the bispecific antibody to block PD-L1 PD1 interaction and thereby promote activation of T cells in autologous co-culture of T and B lymphocytes from peripheral blood samples. The effects of STIM001_289, STIM003_289 and IgG1_289 mAb² antibodies on IFN-γ production were compared to those of anti-PD-L1 AbV, STIM003 and IgG1 monoclonal antibodies in a co-culture of purified peripheral blood monocytes and CD45RO+ memory T-cells from the same donor. These cultures were done in the presence of anti-CD3 antibody to provide TCR stimulation. Retention of ability to promote T cell activation in this assay was confirmed for STIM001 and STIM003 in the ICOS/PD-L1 mAb² bispecific format in which the Fcab region binds human PD-L1. The control PD-L1 mAb², lacking an ICOS binding site, was inactive in this assay.

### Methods

PBMC were isolated from human peripheral blood as described in Example 5c and stored in nitrogen for further utilisation.

STIM001_289, STIM003_289, IgG1_289, anti-PD-L1 AbV, STIM003 and their isotype control IgG1 were serially diluted 1:4 in R10 media to give final 4× antibody concentrations ranging from 40 nM to 40 PM (6-point curve). Anti-human CD3 (clone UCHT1 from eBioscience) was diluted in R10 media to a 4×Ab concentration of 2 μg/ml.

Monocytes and memory cells were isolated from frozen PBMC from the same donor. Monocytes were negatively isolated using the Pan Human Monocyte Isolation Kit (Miltenyi biotec) and resuspended at 2×10⁶/ml (4×) in R10 media. CD45RO+ T-cells were isolated by a first round of negative selection for CD3⁺ T cells (Pan T-cell isolation kit, Miltenyi Biotec), followed by a positive selection for CD45RO+ cells (Human CD45RO MicroBeads, Miltenyi Biotec). CD45RO+ T-cells were then resuspended at 2×10⁶/ml (4×) in R10 media. Cell subsets were co-cultured for 4-days at 37° C. and 5% CO2 at a 1:1 ratio in R10 media in the presence of anti-CD3 (0.5 μg/ml final concentration) and the antibodies under investigation (from 10 nM to 10 pM final concentration). In some wells, no antibody under investigation was added to be able to quantify the basal IFN-γ level (Monocytes+ T-cells+CD3 only). After 4-days culture, cell-free supernatants were analysed for IFN-γ release with the R&D Systems™ Human IFNγ Duoset® ELISA, using DELFIA® Eu-N1 Streptavidin detection. Fold increase in IFN-γ was calculated as: (Experimental IFN-γ level/Basal IFN-γ level) This experiment was repeated with monocytes and T-cells isolated from 7 independent donors and 3 to 5 technical replicates were included for each assay condition (dependent of the number of cells available).

### Results

The levels of IFN-γ induced by STIM001_289, STIM003_289, IgG1_289, STIM003, PD-L1 AbV and their isotype control (IgG1) were measured in assay replicates and plotted as mean value±standard deviation (SD) against the log of antibody concentrations, as shown for one donor (FIG. 14). Unlike STIM003, the antibodies binding PD-L1 such as STIM001_289, STIM003_289, IgG1_289 and anti-PD-L1 AbV all increased the levels of IFN-γ in a concentration dependent manner. This experiment was repeated in 7 independent donors and high variability in IFN-γ production was noticed in absence of antibody under investigation: 311±177 μg/ml (mean±SD, n=7). Levels of IFN-γ obtained were then normalized by the production of IFN-γ in absence of antibody under investigation. The maximum increase in IFN-γ (values at 10 nM) for all 7 donors was plotted and analysed using Friedman statistic test (FIG. 14). The mean increase in IFN-γ level compared to the isotype control (IgG1) induced by was significant for anti-PD-L1 AbV, STIM003_289 and STIM001_289 and was close to significance for IgG1_289 which also binds to PD-L1. Altogether this data is confirming that the PD-L1 binding sites in the bispecific antibodies retain ability to block PD-1/PD-L1 interaction and can subsequently activate T cells.

### Example 10 Anti-Tumour Efficacy of ICOS/PD-L1 Bispecific Antibody on J558 Myeloma In Vivo

The J558 syngeneic tumour model was used to assess the effect of bispecific anti-ICOS/anti-PD-L1 antibodies on myeloma. Two ICOS/PD-L1 mAb² bispecific antibodies, STIM001_457 and STIM003_457, were tested in Balb/c mice using the sub-cutaneous J558 plasmacytoma: myeloma cell line (ATCC, TIB-6), to determine how STIM001_457 hIgG1 and STIM003_457 hIgG1 affect the growth of the tumour.

### Method

Balb/c mice were supplied by Charles River UK at 6-8 weeks of age and >18g and housed under specific pathogen-free conditions. A total of 5×10⁶ cells (passage number below P15) were subcutaneously injected (in 100 μl) into the right flanks of mice. Unless stated otherwise, on day 11 post tumour cells injection, the animals were randomised based on tumour size and treatments were initiated. The J558 cells were passaged in vitro by using TrypLE™ Express Enzyme (Thermofisher), washed twice in PBS and resuspended in DMEM supplemented with 10% foetal calf serum. Cell viability was confirmed to be above 90% at the time of tumour cell injection.

Treatment was initiated when the tumours reached an average volume of ˜140 mm\hat{ }3. Animals were then allocated to 3 groups with similar average tumour size (see Table E10-1 below for the dosing groups). Both bispecific antibodies recognise mouse ICOS (Fab portion) and mouse PD-L1 (Fcab portion) and were dosed IP (dosed at 200 μg per dose) from day 11 (post tumour cell implantation) twice a week for 3 weeks unless the animals had to be removed from study due to welfare (rare) or tumour size. As a control, a group of animals (n=10) was dosed at the same time using a saline solution. Tumour growth was monitored over 37 days and compared to tumours of animals treated with saline. Animal weight and tumour volume were measured 3 times per week from the day of tumour cell injection. Tumour volume was calculated by use of the modified ellipsoid formula ½(Length×Width2). Mice were kept on studies until their tumour reached an average diameter of 12 mm3 or, rarely, when incidence of tumour ulceration was observed (welfare).

TABLE E10-1
Treatment groups for the J558 efficacy study.

| | | Treatment regimen twice per |
|---|---|---|
| Groups | Number of animals | week from day 11 (Mon/Fri) |
| 1 | 10 | Saline |
| 2 | 8 | STIM003_457 200 ug per dose |
| 3 | 8 | STIM001_457 200 ug per dose |

### Results

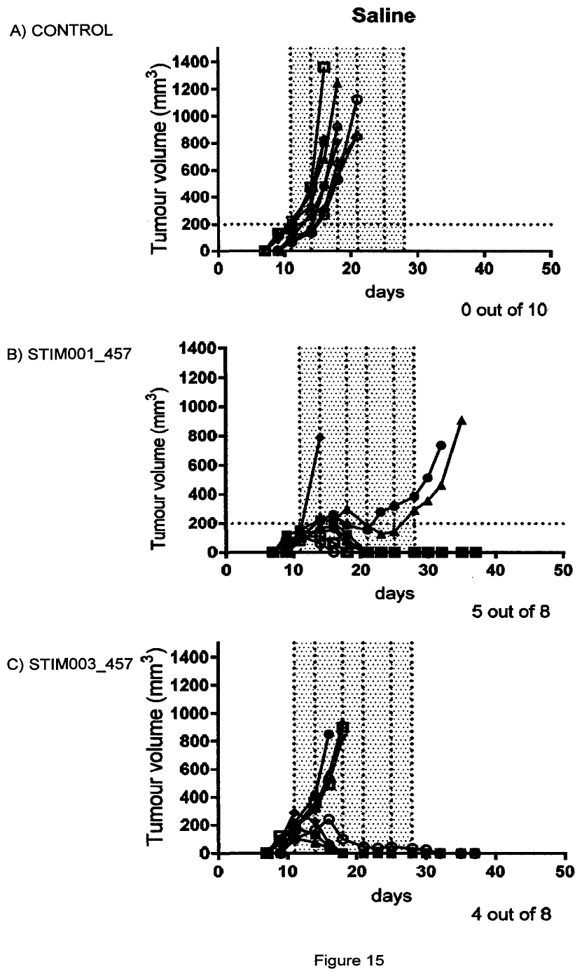

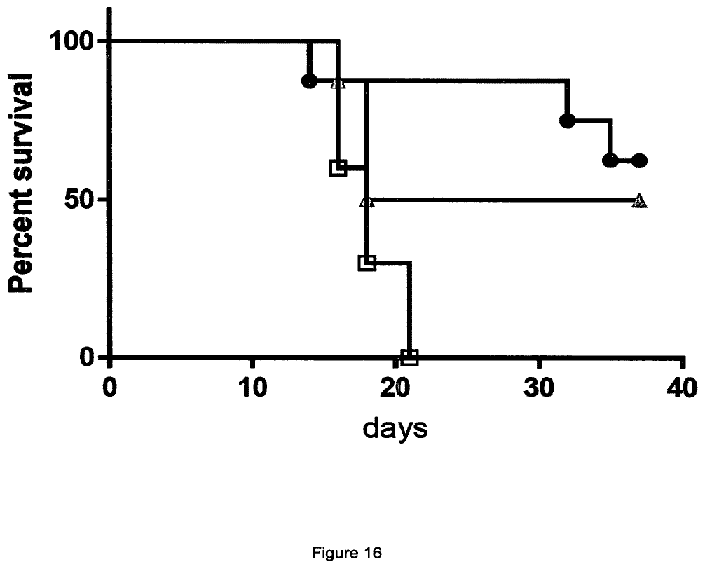

The J558 syngeneic model is highly aggressive. All animals in the saline control group (n=10) had to be removed from studies by day 21 due to tumour size. However, both bispecific antibodies STIM001_457 and STIM003_457 demonstrated good efficacy when used as the sole therapy in this model with, respectively, 50% and 62.5% of the animals cured from their disease by day 37. The anti-tumour efficacy of both bispecific antibodies resulted in improved overall survival of the treated animals (time on study) which was significant vs saline treated group for both antibodies (p<0.05 for STIM003_457 and p<0.001 for STIM001_457). See FIG. 15 and FIG. 16. Table E10-2 below shows hazard ratio (logrank) and p value for the different treatment comparisons.

TABLE E10-2

| | P value (Log Rank, | Hazard Ratio |
|---|---|---|
| Conditions | Mantel-Cox) | (logrank) |
| Saline vs STIM003_457 | P <0.05 | 2.622 |
| Saline vs STIM001_457 | P <0.001 | 5.011 |
| STIM003_457 vs STIM001_457 | Not Significant | 1.498 |

### Example 11a Anti-Tumour Efficacy of ICOS/PD-L1 Bispecific Antibody on CT26 Tumours In Vivo

This example demonstrates strong anti-tumour efficacy in vivo in a CT-26 syngeneic model by co-targeting ICOS and PD-L1 using a bispecific antibody. Bispecific mAb² STIM001_457 hIgG1 and STIM003_457 hIgG1 were both effective in this study.

### Method

Efficacy studies were performed in BALB/c mice using the sub-cutaneous CT-26 colon carcinoma model (ATCC, CRL-2638). BALB/c mice were supplied by Charles River UK at 6-8 weeks of age and >18g and housed under specific pathogen-free conditions. A total of 1×10E5 CT-26 cells (passage number below P20) were subcutaneously injected into the right flanks of mice. Unless stated otherwise, treatments were initiated at day 6 post tumour cells injection. The CT-26 cells were passaged in vitro by using TrypLET Express Enzyme (Thermofisher), washed twice in PBS and resuspended in RPMI supplemented with 10% foetal calf serum. Cell viability was confirmed to be above 90% at the time of tumour cell injection.

STIM001_457 or STIM003_457 bispecific mAb² antibody was each used as the sole therapeutic agent. These antibodies bind to mouse ICOS via the Fab domains and to mouse PD-L1 via the Fc domain (Fcab). Bispecific antibodies were dosed intraperitoneal (IP) at 200 μg each (1 mg/ml in 0.9% saline) three times per week from day 6 (dosing for 2 weeks between day 6-17) post tumour cell implantation. Tumour growth was monitored and compared with tumours of animals in a saline-treated control group and with an isotype IgG1_457 control mAb² antibody which binds to mouse PD-L1 but does not bind to ICOS.

Animal weight and tumour volume were measured 3 times per week from the day of tumour cell injection. Tumour volume was calculated by use of the modified ellipsoid formula ½(Length×Width2). Mice were kept on studies until their tumour reached an average diameter of 12 mm3 or, rarely, when incidence of tumour ulceration was observed (welfare). Mice were re-challenged at day 50. The humane endpoint survival statistics were calculated using the Kaplan-Meier method with Prism. This approach was used to determine if specific treatments were associated with improved survival.

TABLE 11a-1
Bispecific antibody treatment groups in the CT26 model.

| | Number of | Treatment regimen (dosed I.P. 3 time |
|---|---|---|
| Groups | animals | a week from day 6 for 2 weeks) |
| 1 | 10 | Saline |
| 2 | 10 | IgG1_457 Fc effector-disabled (LAGA) |
| | | control 200 μg per dose |
| 3 | 10 | STIM003_457 200 μg per dose |
| 4 | 10 | STIM001_457 200 μg per dose |

### Results

The present experiment clearly demonstrates that both bispecific antibodies significantly delayed tumour growth and extended the survival (time to reach humane endpoint/time on study) of treated animals when compared to saline or IgG1_487 LAGA treated animals. When administered individually as monotherapies rather than in combination, anti-ICOS and anti-PD-L1 antibodies were each significantly less effective at preventing CT26 tumours compared with the bispecific antibodies (data not shown).

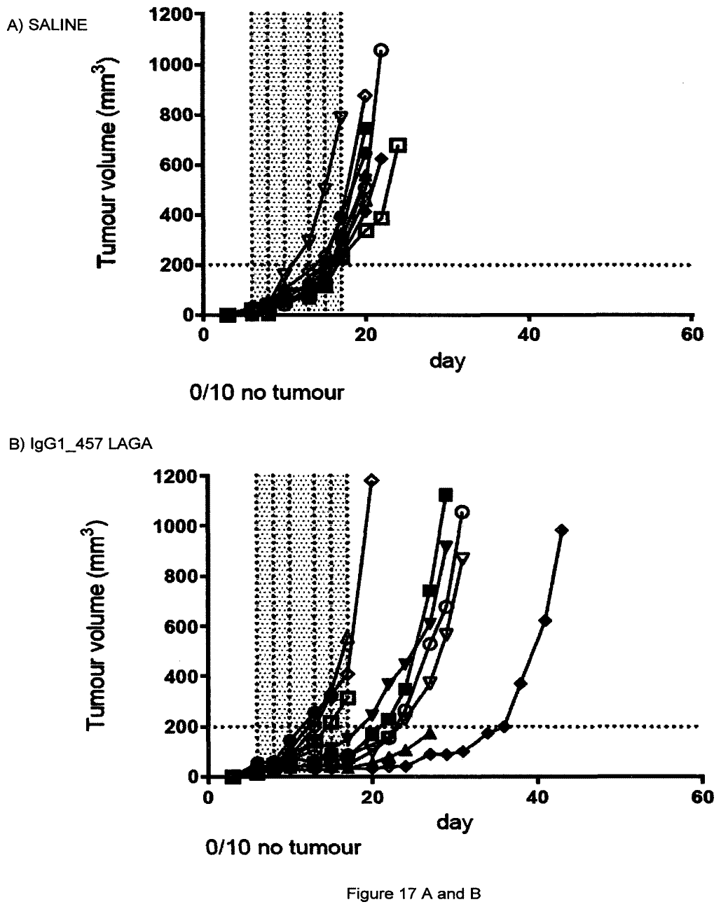

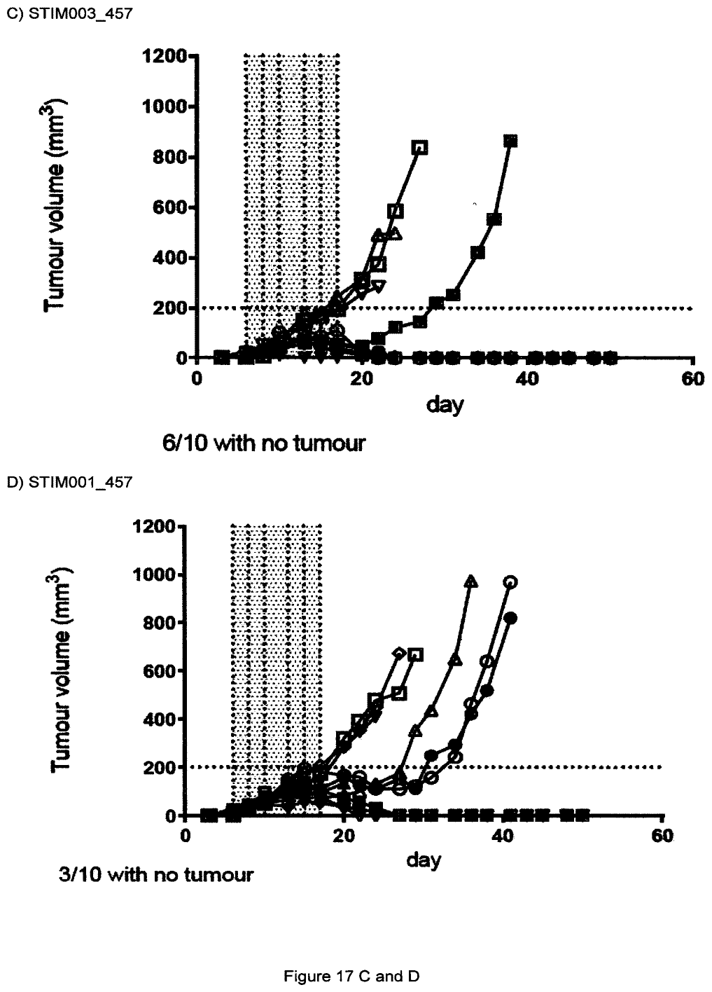

In this experiment STIM003_457 antibody was more effective at inhibiting tumour growth than STIM001_457. STIM003_457 demonstrated the strongest anti-tumour efficacy and improved survival (60% were cured from the disease at day 50) whereas STIM001_457 resulted in 3 out 10 animals (30%) with no sign of disease at the end of the study (day 50). See FIG. 17.

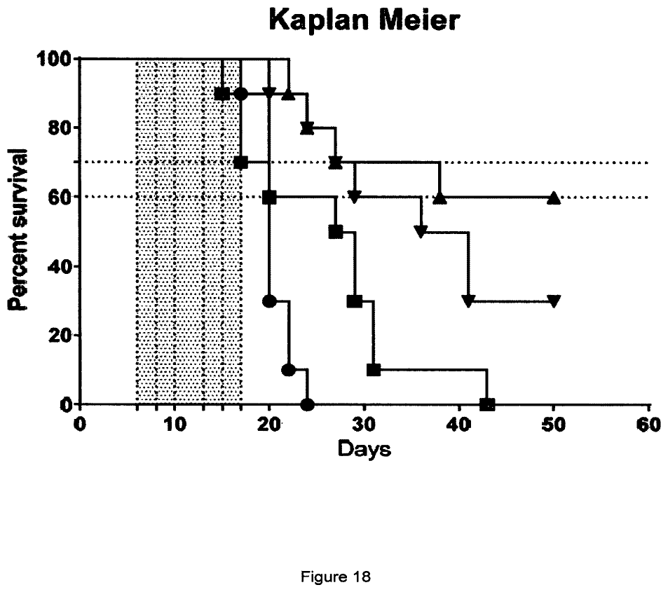

The humane endpoint survival statistics were calculated using the Kaplan-Meier method with Prism. This approach was used to determine if specific treatments were associated with improved survival. See FIG. 18.

TABLE 11a-2

| | P value Log-rank | Hazard Ratio (Mantel- |
|---|---|---|
| conditions (A vs B) | (Mantel-Cox) test | Haenszel A vs B) |
| Saline vs STIM003_457 | P < 0.0001 | 19.71 |
| saline vs STIM001_457 | P < 0.0001 | 17.55 |
| STIM003_457 vs | 0.2831 | 0.5155 |
| STIM001_457 | (not significant) | |

These data confirm that co-targeting ICOS and PD-L1 using a bispecific antibody, even as the sole treatment agent, is effective to trigger an anti-tumour response in the CT26 model.

### Example 11b Treatment with Bispecific ICOS/PD-L1 Antibody Induces Long-Term Immune Memory to Tumour Antigens

Animals that showed full tumour regression following treatment with the bispecific antibodies in Example 11a were challenged again with tumour cells. Of a total of 9 animals, 5 were re-challenged with CT26 to determine whether the animals' immune system showed a memory response to CT26 cells that could prevent these tumours from growing post re-challenge implantation. In addition, 4 animals were challenged with implanted EMT-6 tumour cells, which their immune systems had not been exposed to previously.

TABLE 11b
Bispecific antibody re-challenge groups

| Animal ID | Previous | Cells implanted |
|---|---|---|
| number | treatment groups | (re-challenge) |
| CB43 | STIM003_457 | CT-26 |
| FCA5 | STIM003_457 | CT-26 |
| 1147 | STIM003_457 | CT-26 |
| FEDD | STIM001_457 | CT-26 |
| A6E7 | STIM001_457 | CT-26 |
| E692 | STIM003_457 | EMT-6 |
| C687 | STIM003_457 | EMT-6 |
| D063 | STIM003_457 | EMT-6 |
| FC52 | STIM001_457 | EMT-6 |

Results are shown in FIG. 19. All the animals that had rejected the CT-26 tumours in response to one of the two ICOS/PD-L1 bispecifics managed to reject the newly-injected CT-26 cells in the absence of further treatment. On the other hand, animals that were injected with the new cell line EMT-6, all quickly demonstrated the presence of an established EMT-6 tumour. Altogether the data demonstrate that animals that previously rejected CT-26 tumour in response to either of the bispecific antibody treatments were fully resistant to the CT-26 tumour but not to the unrelated EMT6 tumour. This result indicates that the mice cured by the bispecific antibody therapy had established a long-term memory to tumour antigens expressed specifically by the CT-26 tumour.

### Example 12 Anti-Tumour Efficacy of ICOS-PD-L1 Bispecific Antibody on A20 Tumours In Vivo

ICOS/PD-L1 bispecific antibodies STIM001_457 and STIM003_457 showed strong anti-tumour efficacy in vivo in the A20 syngeneic model when used as sole therapy.

### Method

Efficacy studies were performed in BALB/c mice using the sub-cutaneous A20 Reticulum Cell Sarcoma model (ATCC number CRL-TIB-208). BALB/c mice were supplied by Charles River UK at 6-8 weeks of age and >18g and housed under specific pathogen-free conditions. A total of 5×10E5 A20 cells (passage number below P20) were subcutaneously injected into the right flanks of mice. Unless stated otherwise, treatment was initiated at day 8 post tumour cells injection. The A20 cells were passaged in vitro by using TrypLET Express Enzyme (Thermofisher), washed twice in PBS and resuspended in RPMI supplemented with 10% foetal calf serum. Cell viability was confirmed to be above 85% at the time of tumour cell injection.

The antibodies were dosed intraperitoneally (IP) at 200 μg each (1 mg/ml in 0.9% saline) twice per week from day 8 (dosing for 3 weeks between day 8-25, six doses in total) post tumour cell implantation. Tumour growth was monitored and compared to tumours of animals treated with a IgG2a isotype control group and IgG1_457 (anti-PD-L1 control). Animal weight and tumour volume were measured 3 times a week from the day of tumour cell injection. Tumour volume was calculated by use of the modified ellipsoid formula ½(Length×Width2). Mice were kept on study until their tumour reached an average diameter of 12 mm3 or, rarely, when incidence of tumour ulceration was observed (welfare).

TABLE E12-1
Bispecific antibody treatment groups.

| Groups | Number of animals | Treatment regimen |
|---|---|---|
| 1 | 10 | IgG2a isotype control |
| 2 | 10 | IgG1_457 |
| 3 | 10 | STIM003_457 |
| 4 | 10 | STIM001_457 |

### Results

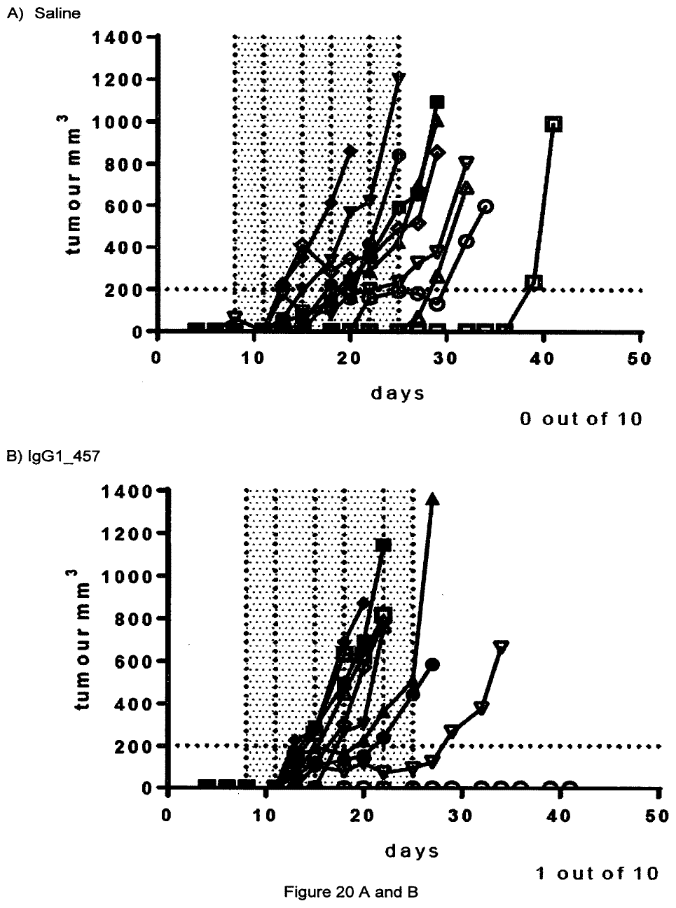

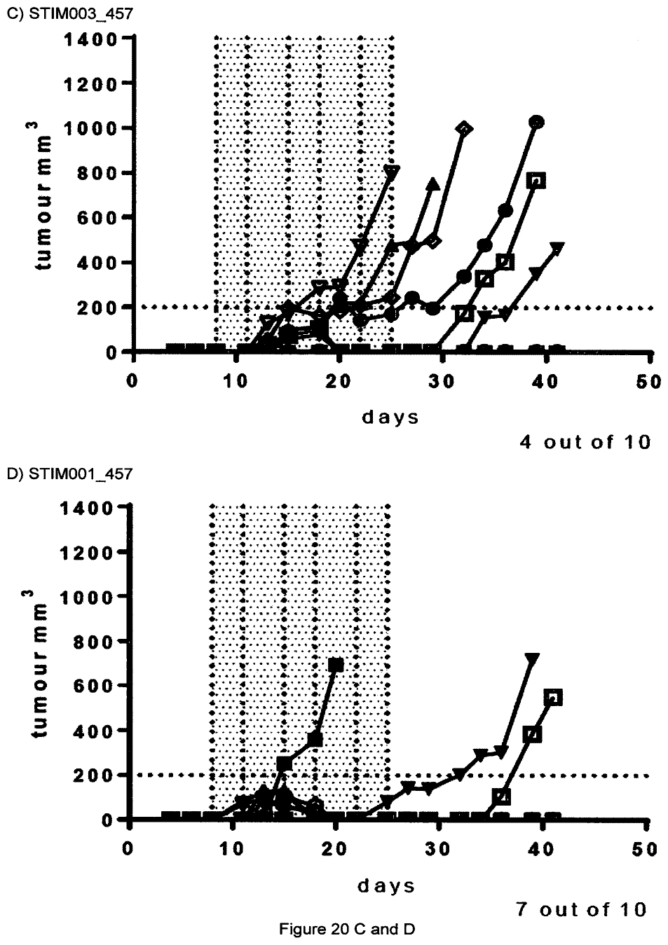

The STIM001_457 and STIM003_457 bispecific antibodies significantly delayed the growth of A20 sub-cutaneous tumours and resulted in extended survival (time to reach humane endpoint) of the treated animals when compared to IgG2a isotype control or IgG1_487 treated animals. All animals in the two control groups had to be removed from the study by day 40. Notably, both bispecific antibodies demonstrated a strong anti-tumour efficacy with 40 and 70% of the animals presented no signs of the disease at day 41. See FIG. 20 and FIG. 21.

### Example 13 Anti-Tumour Efficacy of ICOS/PD-L1 Bispecific Antibody on EMT6 Tumours In Vivo

Anti-tumour in vivo efficacy of co-targeting ICOS and PD-L1 with bispecific antibodies was assessed in an EMT-6 syngeneic model using STIM001_457 and STIM003_457.

### Method

Efficacy studies were performed in BALB/c mice using the sub-cutaneous EMT-6 breast carcinoma model (ATCC number CRL-2755). BALB/c mice were supplied by Charles River UK at 6-8 weeks of age and >18g and housed under specific pathogen-free conditions. A total of 2.5×10E5 EMT-6 cells (passage number below P20) were subcutaneously injected into the right flanks of mice. Unless stated otherwise, treatments were initiated at day 6 post tumour cells injection. The EMT-6 cells were passaged in vitro by using TrypLE™ Express Enzyme (Thermofisher), washed twice in PBS and resuspended in Waymouths MB 752/1 with 2 mM L-glutamine and supplemented with 15% foetal calf serum. Cell viability was confirmed to be above 90% at the time of tumour cell injection.

STIM001_457 and STIM003_457 were each used as single therapeutic agents, dosed intraperitoneally (IP) at 200 μg (1 mg/ml in 0.9% saline) twice a week from day 6 (dosing for 3 weeks between day 6-23) post tumour cell implantation. Tumour growth was monitored and compared to tumours of control animals treated with a saline and IgG1_457 LAGA control. IgG1_457 LAGA can bind PD-L1 and block PD1-PD-L1 interaction but does not bind to ICOS. Animal weight and tumour volume were measured 3 times per week from the day of tumour cell injection. Tumour volume was calculated by use of the modified ellipsoid formula ½(Length×Width2). Mice were kept on studies until their tumour reached an average diameter of 12 mm3 or, rarely, when incidence of tumour ulceration was observed (welfare).

TABLE E13-1
Bispecific antibody treatment groups.

| Groups | Number of animals | Treatment regimen |
|---|---|---|
| 1 | 10 | Saline |
| 7 | 10 | IgG1_457 Fc effector-disabled |
| | | (LAGA) mAb² 200 ug per dose |
| 8 | 10 | STIM003_457 200 ug per dose |
| 9 | 10 | STIM001_457 200 ug per dose |

### Results

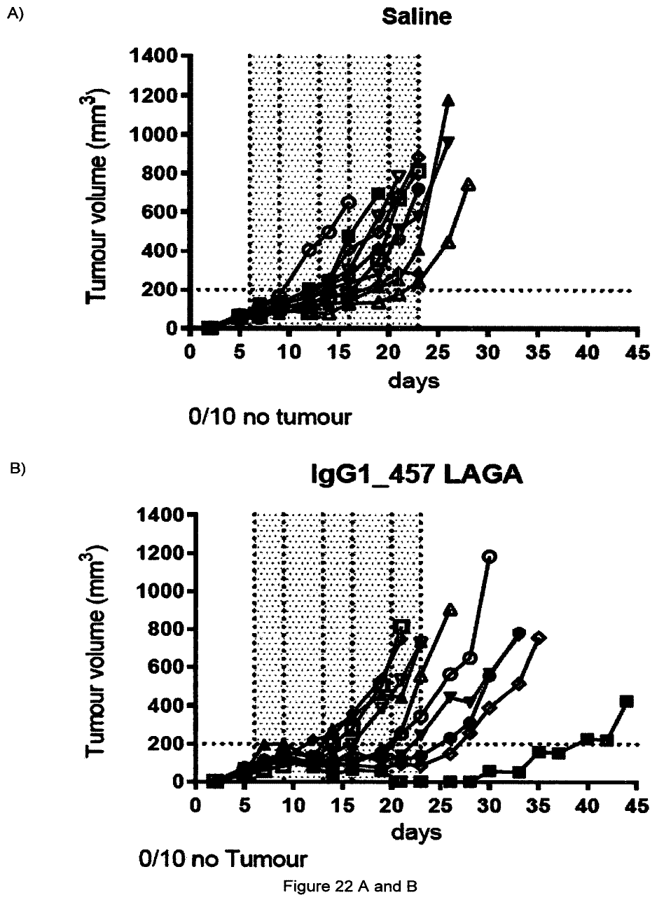

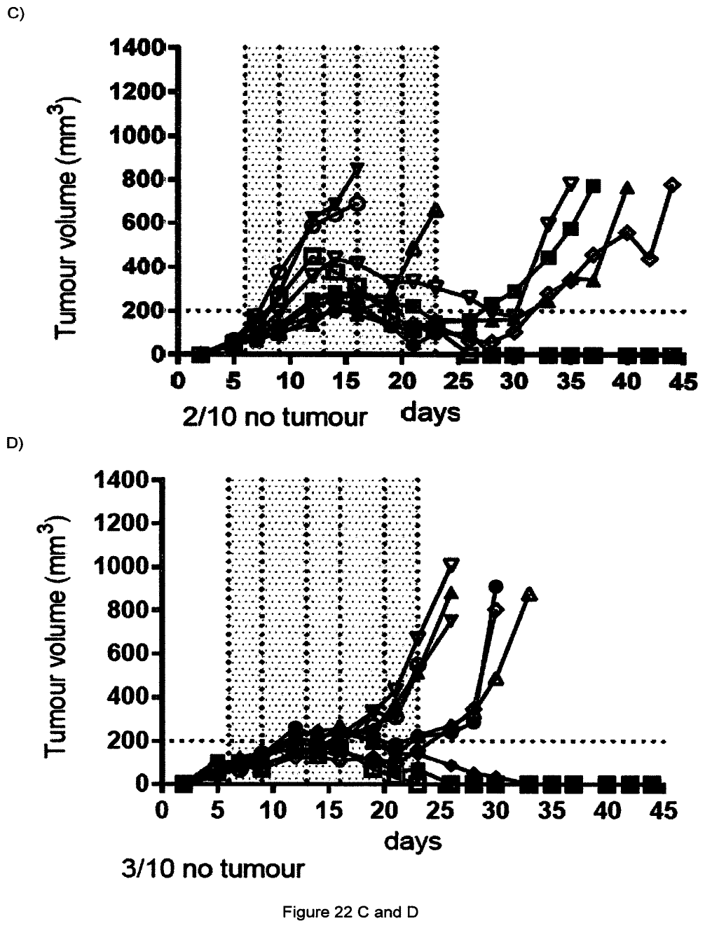

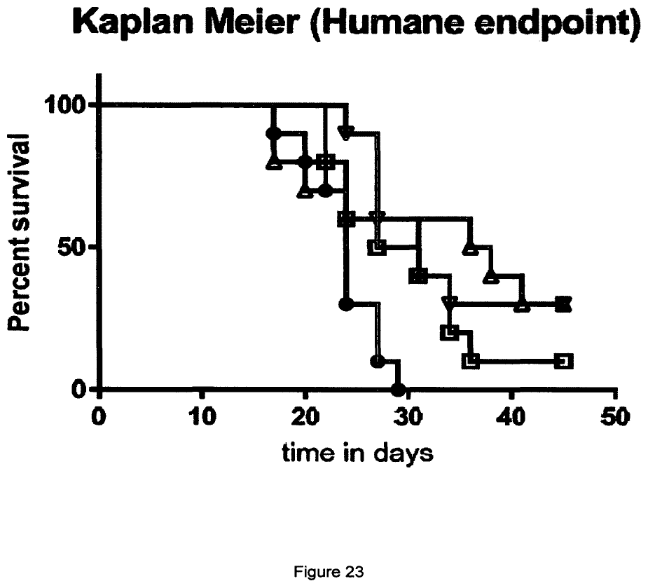

Data are shown in FIG. 22 and FIG. 23, and in Table E13-2 below.

Both bispecific antibodies significantly delayed EMT6 tumour growth and resulted in a longer survival (time to reach humane endpoint) when compared with animals treated with saline or IgG1_457 LAGA. STIM001_457 was marginally more potent in this model and resulted in the strongest anti-tumour efficacy and improved survival (30% vs 20% were cured from the disease at day 44 for STIM001_457 and STIM003_457, respectively), however this difference is not significant. Interestingly the IgG1_457 LAGA treatment resulted in tumour growth delay (vs saline treated group) but unlike what was observed for the ICOS/PD-L1 bispecific, this efficacy did not result in complete response (i.e., absence of tumour at the end of the experiment).

TABLE E13-2
P value and the hazard ratio for different
treatment comparisons.
The statistics were calculated with Prism.

| | P value Log- | Hazard Ratio |
|---|---|---|
| | rank (Mantel- | (Mantel-Hae- |
| Conditions (A vs B) | Cox) test | nszel A vs B) |
| Saline vs STIM003_457 200 ug | 0.0304 | 3.676 |
| Saline vs STIM001_457 200 ug | 0.0009 | 8.683 |
| IgG1_457 LAGA 200 ug vs | 0.2641 | 1.839 |
| STIM003_457 200 ug | | |
| IgG1_457 LAGA 200 ug vs | 0.3174 | 1.741 |
| STIM001_457 200 ug | | |
| STIM003_457 200 ug vs | 0.9692 | 1.022 |
| STIM001_457 200 ug | | |

Comparing the ICOS/PD-L1 bispecific mAb² antibodies with two separate monoclonal antibodies (anti-ICOS STIM003 and anti-PD-L1 antibody), the bispecific mAb² antibodies showed efficacy where the combination did not. See FIG. 24 and FIG. 25.

### Example 14 Anti-ICOS Agonism of MJ T Cells in PD-L1-Dependent Assay

In this ICOS-dependent T cell activation assay, the agonistic potentials of STIM001_289 and STIM003_289 bispecific antibodies were compared with those of STIM001 and STIM003 IgG1 monoclonal antibodies as well as the isotype controls of both the bispecific and monoclonal antibodies in a cell stimulation assay. ICOS expressing MJ cells were stimulated and IFN-γ at 72 hrs post-activation was measured as the readout. This assay can be used to assess the ability of the PD-L1 binding site of the bispecific antibody to present the antibody in a way that promotes activation of target cells through ICOS signalling.

PDL1-dependent release of IFN-γ by the target cells could only be seen with the molecules that bind both ICOS and PDL1 (B7-H1). There was no significant IFN-γ release above background for any of the antibodies when these were added to plates pre-coated with the negative control (BSA). When the positive control Goat anti-human IgG Fcg fragment specific F(ab′)₂, which binds the Fc-domains of both the bispecific and the IgG1 monoclonal antibodies, was coated on the plates, all antibodies that bind ICOS, i.e. STIM001_289, STIM003_289, STIM001 IgG1 and STIM003 IgG1 could induce IFN-γ release, whereas the isotype controls could not. When the plates were coated with PDL1-Fc, only STIM001_289 and STIM003_289 were able to induce IFN-γ, whereas the HYB. CTRL_289, which is not able to bind ICOS, did not. Similarly, the IgG1s, which are unable to bind PDL1 did not induce the release of IFN-γ. Altogether, this experiment demonstrates that the bispecific antibodies can induce PDL1-dependent ICOS agonism.

### Materials and Methods

### Plate Coating

96-well, sterile, flat, high binding plates (Costar) were coated in duplicate overnight at 4° C. with 100 μl/well of DPBS (Gibco) containing either 1% w/v of bovine serum albumin (BSA; Sigma) or 10 μg/ml of recombinant human B7-H1-Fc chimera (RnD Systems) or 10 μg/ml goat anti-human IgG Fcg fragment specific F(ab′)₂ (Jackson ImmunoResearch). Plates were then washed twice with 200 μl/well of DPBS and blocked with 1% BSA for 1 hr at room temperature (RT). The plates were then washed again twice with 200 μl/well of DPBS before the addition of serial dilutions of antibodies.

### Antibody Addition

Serial 1:3 dilutions of STIM001, STIM003 and HYB. CTRL (isotype control) as an IgG1 and STIM001_289, STIM003_289 and HYB. CTRL_289 from 10 μg/ml to 0.51 ng/ml were prepared in 1% BSA/DPBS, added to the plates and agitated for 1.5 hrs at RT.

The plates were washed again 2× with PBS before the addition of MJ cells. To account for background, several wells of the plate were left empty and to enable calculation of percent effect several wells were stimulated with the Cell Stimulation Cocktail ( 1/20×; eBioscience).

### Cell Stimulation

MJ [G11] cell line (ATCC© CRL-8294™) was grown in IMDM (Gibco or ATCC) supplemented with 20% heat inactivated FBS. The cells were counted and 10000 cells/well (100 μl/well) of cell suspension was added to the protein coated plates. Cells were cultured in the plates for 3 days at 37° C. and 5% CO2. Cells were separated from the media by centrifugation and the supernatants collected for IFN-γ content determination.

### Measuring IFN-γ Levels

The IFN-γ content in each well was determined using a modification of the Human IFN-gamma DuoSet ELISA kit (R&D systems). Capture antibody (50 μl/well) was coated overnight at 4 μg/ml in DPBS on black flat bottom, high binding plates (Greiner). The wells were washed three times with 200 μl/well of DPBS+0.1% Tween. The wells were blocked with 200 μl/well of 1% BSA in DPBS (w/v), washed three times with 200 μl/well of DPBS+0.1% Tween and then 50 μl/well of either the IFN-γ standard solutions in RPMI or neat cell supernatant were added to each well. The wells were washed three times with 200 μl/well of DPBS+0.1% Tween before adding 50 μl/well of the detection antibody at 200 ng/ml in DPBS+0.1% BSA. The wells were washed three times with 200 μl/well of DPBS+0.1% Tween before adding 50 μl/well of streptavidin-europium (Perkin Elmer) diluted 1:500 in Assay buffer (Perkin Elmer). The wells were washed three times with 200 μl/well of TBS+0.1% Tween before developing the assay by adding 50 μl/well of Delfia enhancement solution (Perkin Elmer) and measuring the fluorescence emitted at 615 nm on the EnVision Multilabel Plate Reader.

### Data Analysis

IFN-γ values for each well were extrapolated from the standard curve and the average background levels from media-only wells were subtracted. The percent effect was calculated as the fraction of signal compared to the IFN-γ values obtained from wells stimulated with the Cell Stimulation Cocktail. The percent effect values were then used in GraphPad prism to fit a 4-parameter log-logistic concentration response curve.

### Results

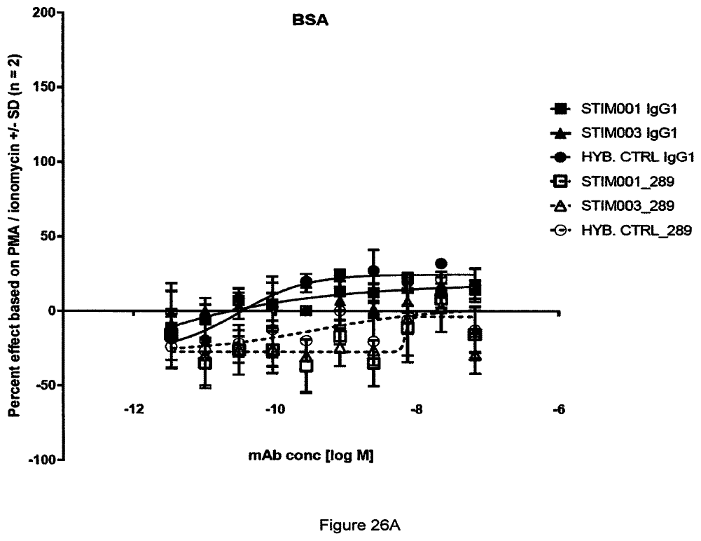

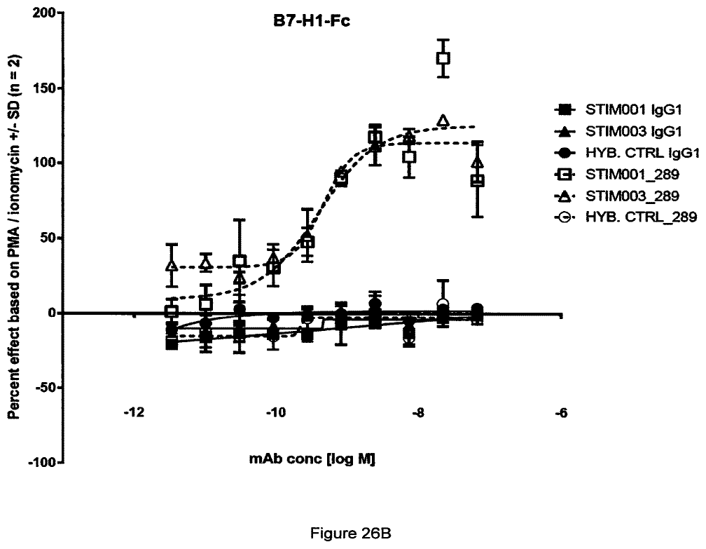

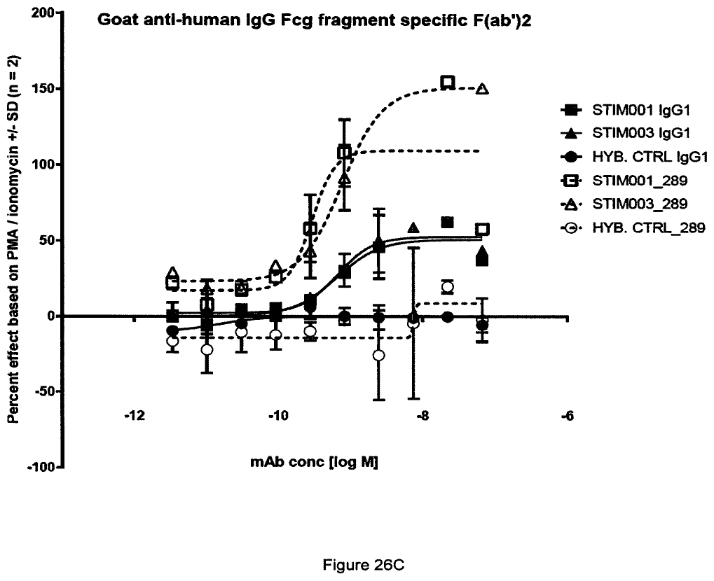

FIG. 26 shows a representative example from two independent experiments.

PDL1-dependent release of IFN-γ by ICOS positive cells could only be seen with the molecules that bind both ICOS and PDL1 (B7-H1). There was no significant IFN-γ release above the background for any of the antibodies when these were added to plates pre-coated with the negative control (BSA). When the positive control Goat anti-human IgG Fcg fragment specific F(ab′)₂ was coated on the plates, all antibodies that bind ICOS, i.e. STIM001_289, STIM003_289, STIM001 IgG1 and STIM003 IgG1 could induce IFN-γ release, whereas the isotype controls could not. When the plates were coated with PDL1-Fc, only STIM001_289 and STIM003_289 were able to induce IFN-γ, whereas the HYB. CTRL_289, which is not able to bind ICOS, did not. Similarly, the IgG1s, which are unable to bind PDL1 did not induce the release of IFN-γ. Altogether, this experiment demonstrates that the bi-specific antibodies can induce PDL1-dependent ICOS agonism.

### Example 15 Formation of Intercellular Bridge by ICOS/PD-L1 Bispecific Antibody

This Example provides data indicating that the mAb² bispecific antibodies of the invention are able to bridge cells expressing ICOS and PD-L1 respectively, in the manner illustrated in FIG. 1.

A flow cytometry protocol was developed to assess the ability of the mAb² STIM001_289 and STIM003_289 to promote the bridging of cells expressing ICOS and cells expressing PD-L1. This experiment aimed to demonstrate that the mAb² can link cells expressing the targets. This data will ultimately be critical to demonstrate that the mAb² can trigger agonism of ICOS+ve cells through cross-presentation using PD-L1 positive cells (such as antigen presenting cells and tumour cells). This PD-L1 dependent ICOS agonism is expected to be important part of the mechanism of action of the mAb² in PD-L1 rich tumour microenvironment. For this purpose, CHO cells expressing human PD-L1 were stained with CellTrace™ Far Red (Invitrogen C34572) which emits at 661 nm while CHO cells expressing human ICOS were stained with CellTrace™ Violet (Invitrogen C34571) which emits at 450 nm. Stained cells were incubated with a titration of mAb² antibodies or a combination of the parental monospecific antibodies and then processed with a flow cytometer.

### Material and Methods

CHO human PD-L1 and CHO human ICOS cells were harvested, counted, washed, and re-suspended in PBS (Gibco 14190169) at 1 million cells per mL. CellTrace™ Far Red and CellTrace™ Violet dyes were diluted 1:2000 and incubated with their respective cells for 20 min at 37° C. in the dark, according to manufacturer recommendations. Buffer (PBS (Gibco 14190169), 1% BSA (Sigma) 0.1% Na Azide (Severn Biotech 40-2010-01)) was then added in excess for an additional 5-minute incubation step. Cells were spun down, re-suspended in the above buffer at 2 million cells per mL and incubated for at least 10 minutes at 37° C. before proceeding with binding protocol. Unstained cells were kept and used to set up the gating strategy.

MAb2 STIM001_289 and STIM003_289, human IgG1 and Hybrid_289 were prepared in buffer at 450 nM and diluted as per 1:3 series, 11 points in triplicates. 50 μL of CHO human PD-L1 cells labelled with CellTrace™ Far Red, 50 μL of CHO human ICOS labelled with CellTrace™ Violet and 50 μL of antibody were added to a 96 well V-bottom PS plate (Greiner 651901).

The monospecific antibodies STIM001 and STIM003, as well as the Hybrid_289 were prepared in buffer at 900 nM and diluted as per 1:3 series, 11 points in triplicates. 25 μL of STIM001 or STIM003 were added to 25 μL of Hybrid_289, 50 μL of labelled CHO human PD-L1 and 50 μL of CHO human ICOS in a 96 well V-bottom PS plate.

Assay plates were incubated at room temperature for 1 hour under gentle agitation (450 rpm) before being read using the Attune N×T flow cytometer (Thermo Fisher) for the detection of fluorescence at 661 nm and 450 nm. FCS files were analysed with FlowJo® software V7.00. Single cells and duplets were gated based on the forward and side scatter dot plot.

### Results

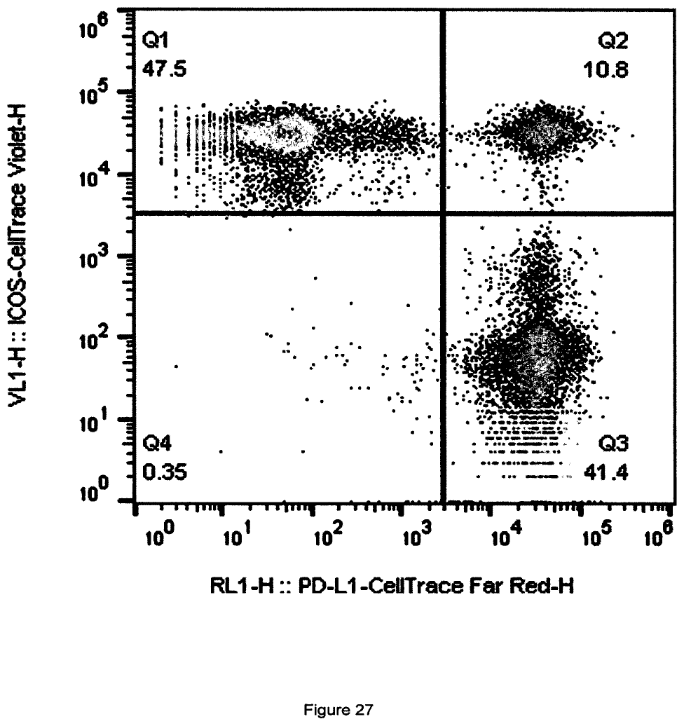

Dot plots graphs (see FIG. 27) resulted in the identification of four different gates: a double negative quadrant corresponding to a very small population (Q4 in FIG. 27) of unstained CHO human PD-L1 and unstained CHO human ICOS; two quadrants positive for one of the two fluorophores (either at 661 nm [PD-L1 CHO cells, Q3 in FIG. 27] or at 450 nm [ICOS CHO cells, Q1 in FIG. 27]); and a quadrant of dual positive staining (at 661 nm and 450 nm, Q2 in FIG. 27) composed of stained PD-L1 and ICOS CHO cells. Cells were seen in this quadrant only when the ICOS/PD-L1 mAb² were used. Percentages of double positive cells were plotted into Prism against antibody titrations.

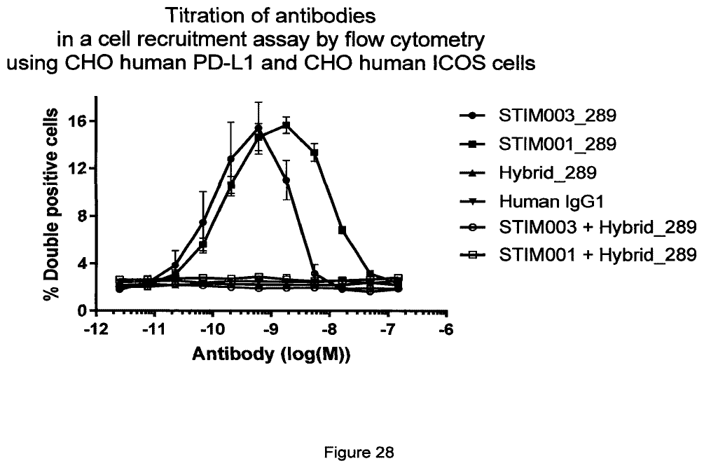

Using as baseline the average percentage of double positive cells obtained with the human IgG1 isotype control, the areas under curve were also calculated for each condition. The highest area under curve was obtained for antibodies able to recruit the most of PD-L1 and ICOS cells and for the widest range of concentrations. See Table E15 and FIG. 28.

TABLE E15

| | Area under curve | |
|---|---|---|
| STIM003_289 | 19.43 | |
| STIM001_289 | 25.45 | |
| Hybrid_289 | 1.22 | |
| Human IgG1 | 0.21 | |
| STIM003 + Hybrid_289 | 2.45 | |
| STIM001 + Hybrid_289 | 0.89 | |
| Baseline | 2.50% | Double positive Cells |

MAb² STIM001_289 and STIM003_289 were able to recruit cells expressing human PD-L1 with cells expressing human ICOS. This data confirmed that the mAb² were able to link/bridge ICOS and PD-L1 positive cells such as effector T cells and APC cells, respectively.

At low concentrations of mAb², there was not enough antibody to bind two cell lines. The maximal percentage of double positive cells was reached when the concentration of mAb² was optimal to recruit the two cell lines (at 1.85 nM for STIM001_289 and at 0.7 nM for STIM003_289). At high concentrations of mAb² antibody, a decrease in the percentage (bell shape curve) of double positive cells was observed. This is expected to be due to saturation of target binding on individual cells by the excess of the antibody. The targets of the CHO human PD-L1 cells and the targets of the CHO human ICOS were both saturated with high mAb² concentration therefore a same mAb² is not able to simultaneously bind two cells.

STIM001_289 could reach the same maximum of double positive cells (˜16%) to STIM003_289, but the double positive signal was observed over a wider range of concentrations for STIM001_289. This was confirmed by a higher area under curve for STIM001_289 than for STIM003_289. This difference may be explained by the different affinities of STIM001 and STIM003 for human ICOS. Altogether, this data confirmed the ability of the mAb² STIM001_289 and STIM003_289 to bridge ICOS and PD-L1 positive cells, such as effector and APC/tumour cells. This demonstrates the potential of triggering PD-L1 dependent cross presentation of the Mab2 to ICOS cells which would be a pre-requisite to PD-L1 dependent ICOS agonism.

### Example 16 Pharmacodynamic Study of Tumour and Spleen Treated with STIM003 457 or STIM001 457 in the CT26-WT Model

### Methods

To ascertain the effects of the bispecific antibodies on certain immune cells in the tumour microenvironment (TME) and peripheral tissue, STIM003_457 and STIM001_457 were given IP twice to CT26-WT tumour-bearing mice. The tumour and spleen were removed for immune cells content analysis by FACS. 24 female BALB/c mice were injected subcutaneously with 0.1×10⁶ cells/mouse of CT26-WT cells, and their tumours allowed to grow. 13 and 15 days post-implantation, mice were dosed intra-peritoneally with either saline, STIM003_457 or STIM001_457 at a fixed dose of 200 μg each. On day 16 post tumour cells-implantation, all mice were culled and tumour, spleen and tumour-draining lymph node (TDLN) were removed for ex vivo analysis. Tumours were dissociated using a mouse tumour dissociation kit (Miltenyi Biotec), followed along with spleen by the MACS gentle dissociator. Spleen cells were incubated for a short period with red blood cell lysis buffer, then all tissues were filtered through 70 μm (tumour) and 40 μm (spleen) cell strainers. The resulting single cell suspensions were washed twice with RPMI+10% FBS complete media, resuspended in FACS buffer and plated into a v-bottomed deep-well 96-well plate. Cells were stained with Live Dead Fixable Yellow viability dye (Life Technologies), followed by washing and an incubation with anti-CD32/CD16 mAb (eBioscience). Afterwards, the following antibodies were added according to three panels: CD3 (17A2), CD45 (30-F11), CD4 (RM4-5), CD8 (53-6.7), CD25 (PC61.5), B220 (RA3-6B2) and ICOS-L (HK5.3), all obtained from eBioscience. Fluorescence-minus-ones were performed in parallel. For staining of intracellular markers (FoxP3), samples were fixed, permeabilized, and stained with FoxP3 mAbs (FJK-16s, eBioscience). Finally, samples were resuspended in PBS and data acquired on the Attune flow cytometer (Invitrogen) and analysed using FlowJo V10 software (Treestar). Gathered data was statistically analysed using the non-parametric Kruskal-Wallis test, followed by post-hoc Dunn's multiple comparisons test (GraphPad Prism V7.0).

### STIM003_457 and STIM001_457 Preferentially Deplete TRegs in the TME

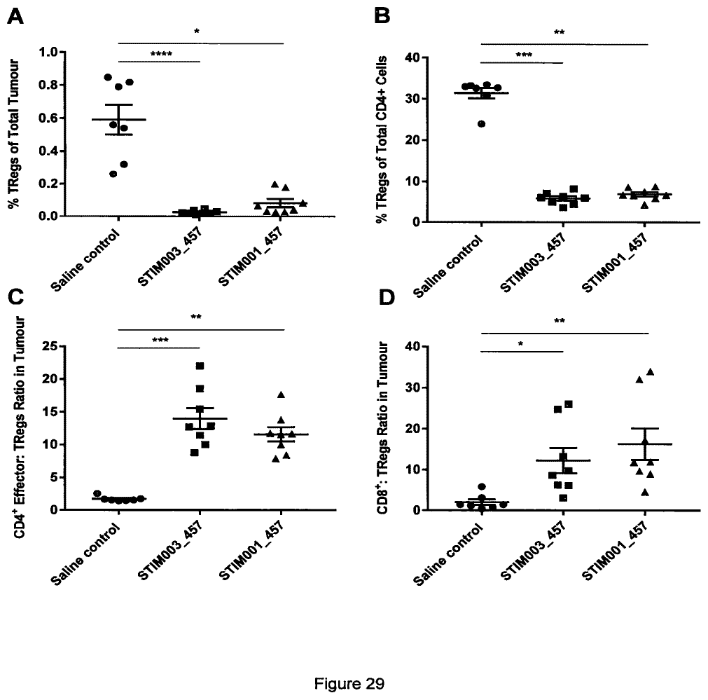

STIM003_457 and STIM001_457 significantly depleted TRegs (defined as CD4⁺ CD25⁺ FoxP3⁺ cells) in the TME when compared to saline (FIGS. 29A and B). There is a clear reduction in both the percentage of TRegs in the whole tumour (all cells), and also as a percentage of total CD4⁺ cells. Correspondingly, we saw a significant increase in the effector CD4 and CD8 T cell: TRegs ratios in the tumour. A high effector T cell: TRegs ratio has been previously associated with increased overall survival in cancer patients and is a key indicator of anti-tumour efficacy for immuno-modulatory molecules. FIG. 29C demonstrates the increase in the number of CD4⁺ effector cells (defined as CD4⁺ CD25 FoxP3 cells) compared to TRegs for both bi-specific antibodies. FIG. 29D shows the same increase in the number of CD8⁺ cells compared to TRegs. The same cell types were investigated in the spleen and as expected, a high/full depletion of TRegs was not observed.

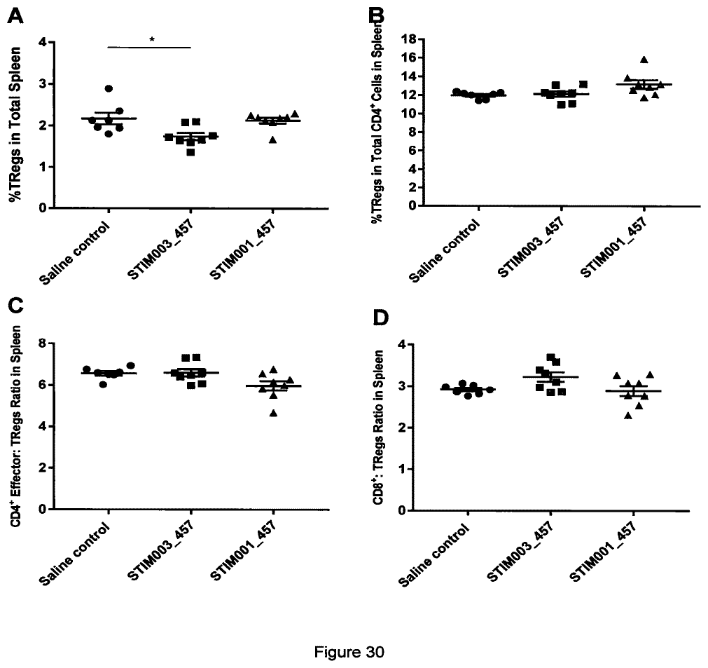

There was only a marginal but yet significant depletion of TRegs in response to STIM003_457 when compared to saline (when TRegs as a percentage of total live cells in the spleen was considered see FIG. 30A). However no significant difference was seen in either antibody group in TRegs as a percentage of total CD4⁺ cells (see FIG. 30B), and there was no significant increase in effector cell to TRegs ratio in response to either bi-specific antibodies in the spleen. Furthermore, examination of the same cells in the tumour-draining lymph node (TDLN) yielded similar results, with no significant difference in any group in any test (results not shown). Overall, (from the 3 tissues analysed), it can be concluded that STIM003_457 and STIM001_457-mediated a strong and significant TRegs depletion selectively in the TME, which results in an increase of effector T-cells to TReg ratio.

### ICOS-L Expression on B-Cells in the Periphery is Increased

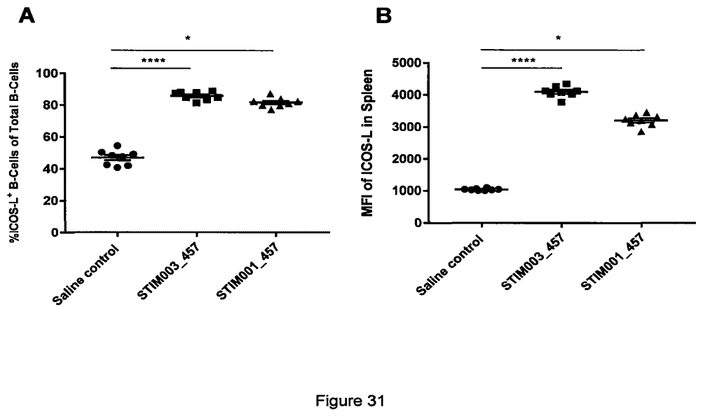

The percentage of B-cells (defined as CD45⁺ B220⁺ cells) which expressed ICOS ligand (ICOS-L) in the spleen was determined by FACS for each treatment group. Both anti-ICOS/anti-PDL1 bispecific antibodies significantly increased the percentage of ICOS-L expressing B-cells by more than 30% when compared to the saline group (see FIG. 31A). The mean fluorescence intensity (MFI) which is used as an indication of ICOS-L relative expression on B-cells was also shown to increase significantly in both groups (see FIG. 31B). In both cases, ICOS-L expression was most increased in animals treated with STIM003_457. Increase in the MFI and in the percentage of ICOS-L-expressing cells indicates that not only was ICOS-L expression more widespread within the B-cell population of antibody-treated animals, but also that expression on each cell was upregulated.

### Example 17 Strong Anti-Tumour In Vivo Efficacy in CT26 Syngeneic Model by Combining STIM003 574 with Anti-PD1 (RMT1-14) or Anti-CTLA-4 (4F10)

Efficacy studies were performed in BALB/c mice using the sub-cutaneous CT26 colon carcinoma model (ATCC, CRL-2638). BALB/c mice were supplied by Charles River UK at 6-8 weeks of age and >18g and housed under specific pathogen-free conditions. A total of 1×10E5 CT26 cells (passage number below P20) were subcutaneously injected into the right flanks of mice. All treatments were initiated at day 6 post tumour cells injection. The CT26 cells were passaged in vitro by using

TrypLE™ Express Enzyme (Thermofisher), washed twice in PBS and resuspended in RPMI supplemented with 10% foetal calf serum. Cell viability was confirmed to be above 90% at the time of tumour cell implantation.

In order to assess how the anti-ICOS/anti-PD-L1 bispecific antibody would combined with an anti-PD1 or an anti-CTLA4, we performed an efficacy study experiment using [STIM003_574 hIgG1⁺/−the PD1 antibody RMT1-14] and [STIM003_574 hIgG1⁺/−the CTLA antibody 4F10]. For the in vivo efficacy studies, STIM003_574 bi-specific (which binds both mouse ICOS and mouse PD-L1 proteins) was also compared to the efficacy of a combination between the two mAbs STIM003 mIgG2a and anti-PD-L1 (AbW). In this experiment the antibodies were administered concomitantly by intraperitoneal (IP) injections. All antibodies were diluted in (1 mg/ml in 0.9% saline) and dosed from day 6 (as shown in table below) three times a week for 2 weeks (between day 6-17) post tumour cell implantation. Tumour growth was monitored and compared to tumours of animals treated with saline. Fixed doses of 200 μg or 60 μg were used which correspond to a dose of 10 mg/kg and 3 mg/kg respectively for mice of 20 g. Tumour volume was calculated by use of the modified ellipsoid formula ½(Length×Width2). Animal weight was also recorded 3 times a week from the day of tumour cell injection. Mice were kept on studies until their tumour reached an average diameter of 12 mm3 or, in rare cases, when incidence of tumour ulceration was observed (welfare). The experiment was stopped at day 46 (40 days after the start of the treatment).

TABLE E17
Treatment groups for CT26 study.

| | Number | Treatments and dose |
|---|---|---|
| | of | (3 times per week for 2 |
| Groups | animals | weeks from day 6) |
| 1 | 8 | Saline |
| 2 | 8 | STIM003_574 hIG1 (200 μg) |
| 3 | 8 | aCTLA4 (4F10) (200 μg) |
| 4 | 8 | aPD-1 (RMT1-14) (200 μg) |
| 5 | 8 | STIM003_574 hIgG1/aCTLA4 |
| | | (4F10) combo (200 μg each) |
| 6 | 8 | STIM003_574 hIgG1/aPD-1 |
| | | (RMT1-14) combo (200 μg each) |
| 7 | 8 | STIM003 mIgG2a (60 μg) + |
| | | anti-PD-L1 (AbW) (200 μg) |

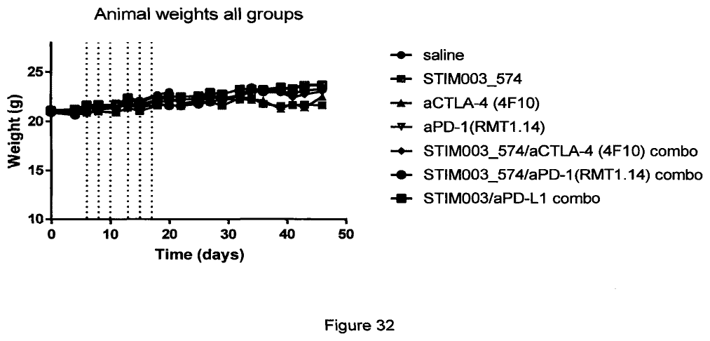

The combination of antibodies targeting immune checkpoint (e.g. anti-PD1+anti-CTLA-4) is often associated with adverse events due to the strong activation of the immune system. In the present experiment some of the groups were effectively receiving triple combinations (targeting ICOS, PD-L1 and PD1 or CTLA-4). We used the average animal weight for each group as a surrogate of tolerability of the different treatments. As shown in FIG. 32, we did not observe a decrease in average weight in any of the groups, confirming that these combinations were well tolerated in the animals.

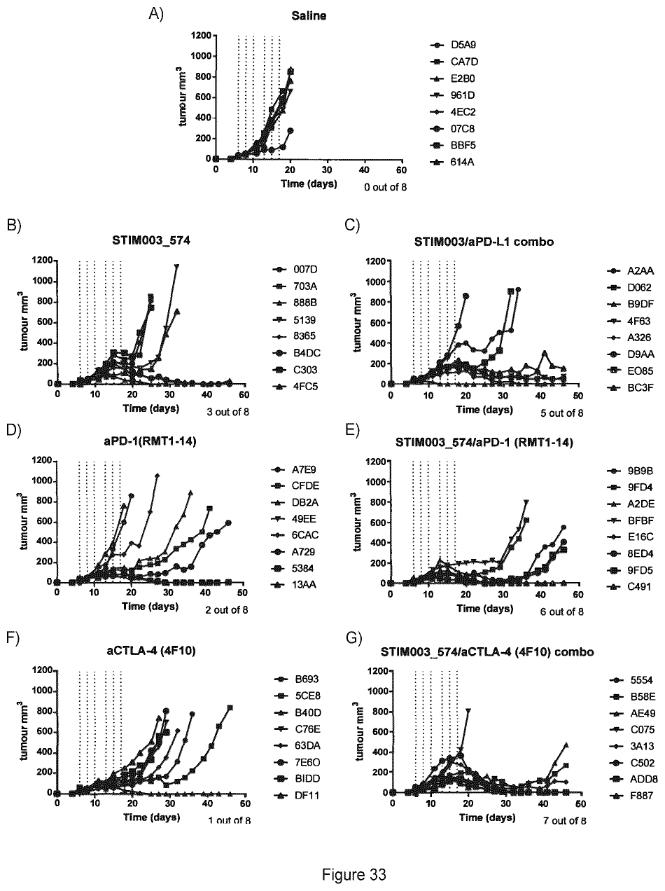

In parallel to monitoring a possible variation in weight, the tumour size for each animal was also measured over 40 days following the start of the treatments (initiated on day 6). As shown in FIG. 33, animals treated with saline had to be sacrificed by day 20 due to tumour size or in one instance due to ulceration of the tumour. On the other hand, the animals treated with STIM003_574 and the animals treated with a combination of STIM003 mIgG2a and anti-PD-L1 (AbW) demonstrated an anti-tumour response with, respectively, 37.5% and 62.5% of the animals still on study by day 46. Importantly the anti-tumour efficacy that we observed with STIM003_574 was significantly improved by combining the bi-specific with anti-PD1 or with anti-CTLA-4. Anti-PD1 and anti-CTLA4 monotherapies could trigger an anti-tumour response but this was only for a short term. In fact, only 1 and 2 animals from the anti-PD1 and the anti-CTLA-4 monotherapy groups were still on study on day 46. Whereas the combination of STIM003_574 with anti-PD1 or anti-CTLA-4 resulted in 6 and 8 animals respectively still on study by the endpoint day.

The STIM003_574 mAb² used in this study is an IgG1 in which the two Fab regions comprise the VH and VL domains of anti-ICOS antibody STIM003 and the Fcab region binds mouse PD-L1. The Fcab regions of _457 and 574 have a minor variation in amino acid sequence. In vitro, the affinity of 457 to mouse PD-L1 is lower compared with that of 574. The affinity of 574 for mouse PD-L1 is closer to the affinity of 289 for human PD-L1. In vivo, 457 and 574 have similar anti-tumour efficacy.

### Sequences

TABLE S1
SEQ ID NOS: 1-342

| SEQ | | | | |
|---|---|---|---|---|
| ID | | | | |
| NO: | Name | Description | | Sequence |
| 1 | Human | NCBI number: | | MRIFAVFIFMTYWHLLNAFTVTVPKDLYVVEYGSNMTIECKFPVEK |
| | PD-L1 | NP_054862.1 | | QLDLAALIVYWEMEDKNIIQFVHGEEDLKVQHSSYRQRARLLKDQL |
| | | (ECD highlighted | | SLGNAALQITDVKLQDAGVYRCMISYGGADYKRITVKVNAPYNKIN |
| | | in BOLD, | | QRILVVDPVTSEHELTCQAEGYPKAEVIWTSSDHQVLSGKTTTTNS |
| | | cytoplasmic domain | | KREEKLFNVTSTLRINTTTNEIFYCTFRRLDPEENHTAELVIPELP |
| | | underlined) | | LAHPPNERTHLVILGAILLCLGVALTFIFRLRKGRMMDVKKCGIQD |
| | | | | TNSKKQSDTHLEET |
| 2 | Cyno PD- | NCBI number: | | MGWSCIILFLVATATGVHSMFTVTVPKDLYVVEYGSNMTIECKFPV |
| | L1 | XP_014973154.1 | | EKQLDLTSLIVYWEMEDKNIIQFVHGEEDLKVQHSNYRQRAQLLKD |
| | | (ECD highlighted | | QLSLGNAALRITDVKLQDAGVYRCMISYGGADYKRITVKVNAPYNK |
| | | in BOLD) | | INQRILVVDPVTSEHELTCQAEGYPKAEVIWTSSDHQVLSGKTTTT |
| | | | | NSKREEKLLNVTSTLRINTTANEIFYCIFRRLDPEENHTAELVIPE |
| | | | | LPLALPPNERT |
| 3 | Human | Human PD-L1 ECD | | MRIFAVFIFMTYWHLLNAFTVTVPKDLYVVEYGSNMTIECKFPVEK |
| | PD-L1 | with C-terminal | | QLDLAALIVYWEMEDKNIIQFVHGEEDLKVQHSSYRQRARLLKDQL |
| | His | His tag | | SLGNAALQITDVKLQDAGVYRCMISYGGADYKRITVKVNAPYNKIN |
| | | | | QRILVVDPVTSEHELTCQAEGYPKAEVIWTSSDHQVLSGKTTTTNS |
| | | | | KREEKLFNVTSTLRINTTTNEIFYCTFRRLDPEENHTAELVIPELP |
| | | | | LAHPPNERTHHHHHH |
| 4 | Human | Human PD-L1 ECD | | MRIFAVFIFMTYWHLLNAFTVTVPKDLYVVEYGSNMTIECKFPVEK |
| | PD-L1 Fc | with C-term Fc | | QLDLAALIVYWEMEDKNIIQFVHGEEDLKVQHSSYRQRARLLKDQL |
| | | fusion (in bold) | | SLGNAALQITDVKLQDAGVYRCMISYGGADYKRITVKVNAPYNKIN |
| | | | | QRILVVDPVTSEHELTCQAEGYPKAEVIWTSSDHQVLSGKTTTTNS |
| | | | | KREEKLFNVTSTLRINTTTNEIFYCTFRRLDPEENHTAELVIPELP |
| | | | | LAHPPNERTIEGREPKSCDKTHTCPPCPAPELLGGPSVFLFPPKPK |
| | | | | DTLMISRTPEVTCVVVDVSHEDPEVKFNWYVDGVEVHNAKTKPREE |
| | | | | QYNSTYRVVSVLTVLHQDWLNGKEYKCKVSNKALPAPIEKTISKAK |
| | | | | GQPREPQVYTLPPSRDELTKNQVSLTCLVKGFYPSDIAVEWESNGQ |
| | | | | PENNYKTTPPVLDSDGSFFLYSKLTVDKSRWQQGNVFSCSVMHEAL |
| | | | | HNHYTQKSLSLSPGK |
| 5 | Cyno PD- | Cynomolgus PD-L1 | | MGWSCIILFLVATATGVHSMFTVTVPKDLYVVEYGSNMTIECKFPV |
| | L1 FLAG | ECD with N-term | | EKQLDLTSLIVYWEMEDKNIIQFVHGEEDLKVQHSNYRQRAQLLKD |
| | | FLAG tag | | QLSLGNAALRITDVKLQDAGVYRCMISYGGADYKRITVKVNAPYNK |
| | | | | INQRILVVDPVTSEHELTCQAEGYPKAEVIWTSSDHQVLSGKTTTT |
| | | | | NSKREEKLLNVTSTLRINTTANEIFYCIFRRLDPEENHTAELVIPE |
| | | | | LPLALPPNERTDYKDDDDK |
| 6 | Human | Human PD-1 full | | MGWSCIILFLVATATGVHSLDSPDRPWNPPTFSPALLVVTEGDNAT |
| | PD-1 Fc | length sequence | | FTCSFSNTSESFVLNWYRMSPSNQTDKLAAFPEDRSQPGQDCRFRV |
| | | derived from cDNA | | TQLPNGRDFHMSVVRARRNDSGTYLCGAISLAPKAQIKESLRAELR |
| | | as human Fc fusion | | VTERRAEVPTAHPSPSPRRAGQKLENLYFQGIEGRMDEPKSCDKTH |
| | | | | TCPPCPAPELLGGPSVFLFPPKPKDTLMISRTPEVTCVVVDVSHED |
| | | | | PEVKFNWYVDGVEVHNAKTKPREEQYNSTYRVVSVLTVLHQDWLNG |
| | | | | KEYKCKVSNKALPAPIEKTISKAKGQPREPQVYTLPPSRDELTKNQ |
| | | | | VSLTCLVKGFYPSDIAVEWESNGQPENNYKTTPPVLDSDGSFFLYS |
| | | | | KLTVDKSRWQQGNVFSCSVMHEALHNHYTQKSLSLSP |
| 7 | 84G09 - | Amino acid | | GFTFDDYA |
| | CDRH1 | sequence of CDRH1 | | |
| | (IMGT) | of 84G09 using | | |
| | | IMGT | | |
| 8 | 84G09 - | Amino acid | | ISWKSNII |
| | CDRH2 | sequence of CDRH2 | | |
| | (IMGT) | of 84G09 using | | |
| | | IMGT | | |
| 9 | 84G09 - | Amino acid | | ARDITGSGSYGWFDP |
| | CDRH3 | sequence of CDRH3 | | |
| | (IMGT) | of 84G09 using | | |
| | | IMGT | | |
| 10 | 84G09 - | Amino acid | | DYAMH |
| | CDRH1 | sequence of CDRH1 | | |
| | (Kabat) | of 84G09 using | | |
| | | Kabat | | |
| 11 | 84G09 - | Amino acid | | GISWKSNIIGYADSVKG |
| | CDRH2 | sequence of CDRH2 | | |
| | (Kabat) | of 84G09 using | | |
| | | Kabat | | |
| 12 | 84G09 - | Amino acid | | DITGSGSYGWFDP |
| | CDRH3 | sequence of CDRH3 | | |
| | (Kabat) | of 84G09 using | | |
| | | Kabat | | |
| 13 | 84G09 - | Amino acid | | EVQLVESGGGLVQPGRSLRLSCAASGFTFDDYAMHWVRQTPGKGLE |
| | Heavy | sequence of V_{H} of | | WVSGISWKSNIIGYADSVKGRFTISRDNAKNSLYLQMNSLRAEDTA |
| | chain | 84G09 (mutations | | LYYCARDITGSGSYGWFDPWGQGTLVTVSS |
| | variable | from germline are | | |
| | region | shown in bold | | |
| | | letters) | | |
| 14 | 84G09 - | Nucleic acid | | CAaGAAAAAGCTTGCCGCCACCATGGAGTTTGGGCTGAGCTGGATT |
| | Heavy | sequence of V_{H} of | | TTCCTTTTGGCTATTTTAAAAGGTGTCCAGTGTGAAGTACAATTGG |
| | chain | 84G09 | | TGGAGTCCGGGGGAGGCTTGGTACAGCCTGGCAGGTCCCTGAGACT |
| | variable | | | CTCCTGTGCAGCCTCTGGATTCACCTTTGATGATTATGCCATGCAC |
| | region | | | TGGGTCCGACAAACTCCAGGGAAGGGCCTGGAGTGGGTCTCAGGTA |
| | | | | TAAGTTGGAAGAGTAATATCATAGGCTATGCGGACTCTGTGAAGGG |
| | | | | CCGATTCACCATCTCCAGAGACAACGCCAAGAACTCCCTGTATCTG |
| | | | | CAAATGAACAGTCTGAGAGCTGAGGACACGGCCTTGTATTATTGTG |
| | | | | CAAGAGATATAACGGGTTCGGGGAGTTATGGCTGGTTCGACCCCTG |
| | | | | GGGCCAGGGAACCCTGGTCACCGTCTCCTCAGCCAAAACGACACCC |
| | | | | CCATCTGTCTATCCACTGGCCCCTGAATCTGCTAAAACTCAGCCTC |
| | | | | CG |
| 15 | 84G09 - | Amino acid | | EVQLVESGGGLVQPGRSLRLSCAASGFTFDDYAMHWVRQTPGKGLE |
| | full | sequence of 84G09 | | WVSGISWKSNIIGYADSVKGRFTISRDNAKNSLYLQMNSLRAEDTA |
| | heavy | heavy chain | | LYYCARDITGSGSYGWFDPWGQGTLVTVSSASTKGPSVFPLAPCSR |
| | chain | (mutations from | | STSESTAALGCLVKDYFPEPVTVSWNSGALTSGVHTFPAVLQSSGL |
| | sequence | germline are shown | | YSLSSVVTVPSSSLGTKTYTCNVDHKPSNTKVDKRVESKYGPPCPP |
| | | in bold letters) | | CPAPEFEGGPSVFLFPPKPKDTLMISRTPEVTCVVVDVSQEDPEVQ |
| | | | | FNWYVDGVEVHNAKTKPREEQFNSTYRVVSVLTVLHQDWLNGKEYK |
| | | | | CKVSNKGLPSSIEKTISKAKGQPREPQVYTLPPSQEEMTKNQVSLT |
| | | | | CLVKGFYPSDIAVEWESNGQPENNYKTTPPVLDSDGSFFLYSRLTV |
| | | | | DKSRWQEGNVFSCSVMHEALHNHYTQKSLSLSLGK |
| 16 | 84G09 - | Nucleic acid | | GAAGTGCAGCTGGTGGAATCTGGCGGCGGACTGGTGCAGCCTGGCA |
| | full | sequence of 84G09 | | GATCCCTGAGACTGTCTTGTGCCGCCTCCGGCTTCACCTTCGACGA |
| | heavy | heavy chain | | CTACGCTATGCACTGGGTGCGACAGACCCCTGGCAAGGGCCTGGAA |
| | chain | | | TGGGTGTCCGGCATCTCCTGGAAGTCCAACATCATCGGCTACGCCG |
| | sequence | | | ACTCCGTGAAGGGCCGGTTCACCATCTCCCGGGACAACGCCAAGAA |
| | | | | CTCCCTGTACCTGCAGATGAACAGCCTGCGGGCCGAGGACACCGCC |
| | | | | CTGTACTACTGCGCCAGAGACATCACCGGCTCCGGCTCCTACGGAT |
| | | | | GGTTCGATCCTTGGGGCCAGGGCACCCTCGTGACCGTGTCCTCTGC |
| | | | | CAGCACCAAGGGCCCCTCTGTGTTCCCTCTGGCCCCTTCCAGCAAG |
| | | | | TCCACCTCTGGCGGAACAGCCGCTCTGGGCTGCCTCGTGAAGGACT |
| | | | | ACTTCCCCGAGCCTGTGACCGTGTCCTGGAACTCTGGCGCTCTGAC |
| | | | | CAGCGGAGTGCACACCTTCCCTGCTGTGCTGCAGTCCTCCGGCCTG |
| | | | | TACTCCCTGTCCTCCGTCGTGACCGTGCCTTCCAGCTCTCTGGGCA |
| | | | | CCCAGACCTACATCTGCAACGTGAACCACAAGCCCTCCAACACCAA |
| | | | | GGTGGACAAGAAGGTGGAACCCAAGTCCTGCGACAAGACCCACACC |
| | | | | TGTCCCCCTTGTCCTGCCCCTGAACTGCTGGGCGGACCTTCCGTGT |
| | | | | TCCTGTTCCCCCCAAAGCCCAAGGACACCCTGATGATCTCCCGGAC |
| | | | | CCCCGAAGTGACCTGCGTGGTGGTGGATGTGTCCCACGAGGACCCT |
| | | | | GAAGTGAAGTTCAATTGGTACGTGGACGGCGTGGAAGTGCACAACG |
| | | | | CCAAGACCAAGCCTAGAGAGGAACAGTACAACTCCACCTACCGGGT |
| | | | | GGTGTCCGTGCTGACCGTGCTGCACCAGGATTGGCTGAACGGCAAA |
| | | | | GAGTACAAGTGCAAGGTGTCCAACAAGGCCCTGCCTGCCCCCATCG |
| | | | | AAAAGACCATCTCCAAGGCCAAGGGCCAGCCCCGGGAACCCCAGGT |
| | | | | GTACACACTGCCCCCTAGCAGGGACGAGCTGACCAAGAACCAGGTG |
| | | | | TCCCTGACCTGTCTCGTGAAAGGCTTCTACCCCTCCGATATCGCCG |
| | | | | TGGAATGGGAGTCCAACGGCCAGCCTGAGAACAACTACAAGACCAC |
| | | | | CCCCCCTGTGCTGGACTCCGACGGCTCATTCTTCCTGTACAGCAAG |
| | | | | CTGACAGTGGACAAGTCCCGGTGGCAGCAGGGCAACGTGTTCTCCT |
| | | | | GCTCCGTGATGCACGAGGCCCTGCACAACCACTACACCCAGAAGTC |
| | | | | CCTGTCCCTGAGCCCCGGCAAG |
| 17 | 84G09 - | Amino acid | | QSISSY |
| | CDRL1 | sequence of CDRL1 | | |
| | (IMGT) | of 84G09 using | | |
| | | IMGT | | |
| 18 | 84G09 - | Amino acid | | VAS |
| | CDRL2 | sequence of CDRL2 | | |
| | (IMGT) | of 84G09 using | | |
| | | IMGT | | |
| 19 | 84G09 - | Amino acid | | QQSYSNPIT |
| | CDRL3 | sequence of CDRL3 | | |
| | (IMGT) | of 84G09 using | | |
| | | IMGT | | |
| 20 | 84G09 - | Amino acid | | RASQSISSYLN |
| | CDRL1 | sequence of CDRL1 | | |
| | (Kabat) | of 84G09 using | | |
| | | Kabat | | |
| 21 | 84G09 - | Amino acid | | VASSLQS |
| | CDRL2 | sequence of CDRL2 | | |
| | (Kabat) | of 84G09 using | | |
| | | Kabat | | |
| 22 | 84G09 - | Amino acid | | QQSYSNPIT |
| | CDRL3 | sequence of CDRL3 | | |
| | (Kabat) | of 84G09 using | | |
| | | Kabat | | |
| 23 | 84G09 - | Amino acid | | DIQMTQSPSSLSASVGDRVTITCRASQSISSYLNWYQQKPGKAPKP |
| | Light | sequence of V_{L} of | | LIYVASSLQSGVPSSFSGSGSGTDFTLTISSLQPEDFATYYCQQSY |
| | chain | 84G09 | | SNPITFGQGTRLEIK |
| | variable | | | |
| | region | | | |
| 24 | 84G09 - | Nucleic acid | | GACATCCAGATGACCCAGTCTCCATCCTCCCTGTCTGCATCTGTAG |
| | Light | sequence of V_{L} of | | GAGACAGAGTCACCATCACTTGCCGGGCAAGTCAGAGCATTAGCAG |
| | chain | 84G09 | | CTATTTAAATTGGTATCAGCAGAAACCAGGGAAAGCCCCTAAGCCC |
| | variable | | | CTGATCTATGTTGCATCCAGTTTGCAAAGTGGGGTCCCATCAAGTT |
| | region | | | TCAGTGGCAGTGGATCTGGGACAGATTTCACTCTCACCATCAGCAG |
| | | | | TCTGCAACCTGAAGATTTTGCAACTTACTACTGTCAACAGAGTTAC |
| | | | | AGTAATCCGATCACCTTCGGCCAAGGGACACGACTGGAGATCAAA |
| 25 | 84G09 - | Amino acid | | DIQMTQSPSSLSASVGDRVTITCRASQSISSYLNWYQQKPGKAPKP |
| | full | sequence of 84G09 | | LIYVASSLQSGVPSSFSGSGSGTDFTLTISSLQPEDFATYYCQQSY |
| | light | light chain | | SNPITFGQGTRLEIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 26 | 84G09 - | Nucleic acid | | GACATCCAGATGACCCAGTCTCCATCCTCCCTGTCTGCATCTGTAG |
| | full | sequence of 84G09 | | GAGACAGAGTCACCATCACTTGCCGGGCAAGTCAGAGCATTAGCAG |
| | light | light chain | | CTATTTAAATTGGTATCAGCAGAAACCAGGGAAAGCCCCTAAGCCC |
| | chain | | | CTGATCTATGTTGCATCCAGTTTGCAAAGTGGGGTCCCATCAAGTT |
| | sequence | | | TCAGTGGCAGTGGATCTGGGACAGATTTCACTCTCACCATCAGCAG |
| | | | | TCTGCAACCTGAAGATTTTGCAACTTACTACTGTCAACAGAGTTAC |
| | | | | AGTAATCCGATCACCTTCGGCCAAGGGACACGACTGGAGATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 27 | 1D05 - | Amino acid | | GFTFDDYA |
| | CDRH1 | sequence of CDRH1 | | |
| | (IMGT) | of 1D05 using IMGT | | |
| 28 | 1D05 - | Amino acid | | ISWIRTGI |
| | CDRH2 | sequence of CDRH2 | | |
| | (IMGT) | of 1D05 using IMGT | | |
| 29 | 1D05 - | Amino acid | | AKDMKGSGTYGGWFDT |
| | CDRH3 | sequence of CDRH3 | | |
| | (IMGT) | of 1D05 using IMGT | | |
| 30 | 1D05 - | Amino acid | | DYAMH |
| | CDRH1 | sequence of CDRH1 | | |
| | (Kabat) | of 1D05 using | | |
| | | Kabat | | |
| 31 | 1D05 - | Amino acid | | GISWIRTGIGYADSVKG |
| | CDRH2 | sequence of CDRH2 | | |
| | (Kabat) | of 1D05 using | | |
| | | Kabat | | |
| 32 | 1D05 - | Amino acid | | DMKGSGTYGGWFDT |
| | CDRH3 | sequence of CDRH3 | | |
| | (Kabat) | of 1D05 using | | |
| | | Kabat | | |
| 33 | 1D05 - | Amino acid | | EVQLVESGGGLVQPGRSLRLSCAASGFTFDDYAMHWVRQVPGKGLE |
| | Heavy | sequence of V_{H} of | | WVSGISWIRTGIGYADSVKGRFTIFRDNAKNSLYLQMNSLRAEDTA |
| | chain | 1D05 (mutations | | LYYCAKDMKGSGTYGGWFDTWGQGTLVTVSS |
| | variable | from germline are | | |
| | region | shown in bold | | |
| | | letters) | | |
| 34 | 1D05 - | Nucleic acid | | AAGCTTGCCGCCACCATGGAGTTTGGGCTGAGCTGGATTTTCCTTT |
| | Heavy | sequence of V_{H} of | | TGGCTATTTTAAAAGGTGTCCAGTGTGAAGTGCAGCTGGTGGAGTC |
| | chain | 1D05 | | TGGGGGAGGCTTGGTGCAGCCTGGCAGGTCCCTGAGACTCTCCTGT |
| | variable | | | GCAGCCTCTGGATTCACCTTTGATGATTATGCCATGCACTGGGTCC |
| | region | | | GGCAAGTTCCAGGGAAGGGCCTGGAATGGGTCTCAGGCATTAGTTG |
| | | | | GATTCGTACTGGCATAGGCTATGCGGACTCTGTGAAGGGCCGATTC |
| | | | | ACCATTTTCAGAGACAACGCCAAGAATTCCCTGTATCTGCAAATGA |
| | | | | ACAGTCTGAGAGCTGAGGACACGGCCTTGTATTACTGTGCAAAAGA |
| | | | | TATGAAGGGTTCGGGGACTTATGGGGGGTGGTTCGACACCTGGGGC |
| | | | | CAGGGAACCCTGGTCACCGTCTCCTCAGCCAAAACAACAGCCCCAT |
| | | | | CGGTCTATCCACTGGCCCCTGC |
| 35 | 1D05 - | Amino acid | | EVQLVESGGGLVQPGRSLRLSCAASGFTFDDYAMHWVRQVPGKGLE |
| | full | sequence of 1D05 | | WVSGISWIRTGIGYADSVKGRFTIFRDNAKNSLYLQMNSLRAEDTA |
| | heavy | heavy chain | | LYYCAKDMKGSGTYGGWFDTWGQGTLVTVSSASTKGPSVFPLAPCS |
| | chain | | | RSTSESTAALGCLVKDYFPEPVTVSWNSGALTSGVHTFPAVLQSSG |
| | sequence | | | LYSLSSVVTVPSSSLGTKTYTCNVDHKPSNTKVDKRVESKYGPPCP |
| | | | | PCPAPEFEGGPSVFLFPPKPKDTLMISRTPEVTCVVVDVSQEDPEV |
| | | | | QFNWYVDGVEVHNAKTKPREEQFNSTYRVVSVLTVLHQDWLNGKEY |
| | | | | KCKVSNKGLPSSIEKTISKAKGQPREPQVYTLPPSQEEMTKNQVSL |
| | | | | TCLVKGFYPSDIAVEWESNGQPENNYKTTPPVLDSDGSFFLYSRLT |
| | | | | VDKSRWQEGNVFSCSVMHEALHNHYTQKSLSLSLGK |
| 36 | 1D05 - | Nucleic acid | | GAAGTGCAGCTGGTGGAATCTGGCGGCGGACTGGTGCAGCCTGGCA |
| | full | sequence of 1D05 | | GATCCCTGAGACTGTCTTGTGCCGCCTCCGGCTTCACCTTCGACGA |
| | heavy | heavy chain | | CTACGCTATGCACTGGGTGCGACAGGTGCCAGGCAAGGGCCTGGAA |
| | chain | | | TGGGTGTCCGGCATCTCTTGGATCCGGACCGGCATCGGCTACGCCG |
| | sequence | | | ACTCTGTGAAGGGCCGGTTCACCATCTTCCGGGACAACGCCAAGAA |
| | | | | CTCCCTGTACCTGCAGATGAACAGCCTGCGGGCCGAGGACACCGCC |
| | | | | CTGTACTACTGCGCCAAGGACATGAAGGGCTCCGGCACCTACGGCG |
| | | | | GATGGTTCGATACTTGGGGCCAGGGCACCCTCGTGACCGTGTCCTC |
| | | | | TGCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAGG |
| | | | | ACTACTTCCCCGAGCCTGTGACCGTGTCCTGGAACTCTGGCGCTCT |
| | | | | GACCAGCGGAGTGCACACCTTCCCTGCTGTGCTGCAGTCCTCCGGC |
| | | | | CTGTACTCCCTGTCCTCCGTCGTGACCGTGCCTTCCAGCTCTCTGG |
| | | | | GCACCCAGACCTACATCTGCAACGTGAACCACAAGCCCTCCAACAC |
| | | | | CAAGGTGGACAAGAAGGTGGAACCCAAGTCCTGCGACAAGACCCAC |
| | | | | ACCTGTCCCCCTTGTCCTGCCCCTGAACTGCTGGGCGGACCTTCCG |
| | | | | TGTTCCTGTTCCCCCCAAAGCCCAAGGACACCCTGATGATCTCCCG |
| | | | | GACCCCCGAAGTGACCTGCGTGGTGGTGGATGTGTCCCACGAGGAC |
| | | | | CCTGAAGTGAAGTTCAATTGGTACGTGGACGGCGTGGAAGTGCACA |
| | | | | ACGCCAAGACCAAGCCTAGAGAGGAACAGTACAACTCCACCTACCG |
| | | | | GGTGGTGTCCGTGCTGACCGTGCTGCACCAGGATTGGCTGAACGGC |
| | | | | AAAGAGTACAAGTGCAAGGTGTCCAACAAGGCCCTGCCTGCCCCCA |
| | | | | TCGAAAAGACCATCTCCAAGGCCAAGGGCCAGCCCCGGGAACCCCA |
| | | | | GGTGTACACACTGCCCCCTAGCAGGGACGAGCTGACCAAGAACCAG |
| | | | | GTGTCCCTGACCTGTCTCGTGAAAGGCTTCTACCCCTCCGATATCG |
| | | | | CCGTGGAATGGGAGTCCAACGGCCAGCCTGAGAACAACTACAAGAC |
| | | | | CACCCCCCCTGTGCTGGACTCCGACGGCTCATTCTTCCTGTACAGC |
| | | | | AAGCTGACAGTGGACAAGTCCCGGTGGCAGCAGGGCAACGTGTTCT |
| | | | | CCTGCTCCGTGATGCACGAGGCCCTGCACAACCACTACACCCAGAA |
| | | | | GTCCCTGTCCCTGAGCCCCGGCAAG |
| 37 | 1D05 - | Amino acid | | QSISSY |
| | CDRL1 | sequence of CDRL1 | | |
| | (IMGT) | of 1D05 using IMGT | | |
| 38 | 1D05 - | Amino acid | | VAS |
| | CDRL2 | sequence of CDRL2 | | |
| | (IMGT) | of 1D05 using IMGT | | |
| 39 | 1D05 - | Amino acid | | QQSYSTPIT |
| | CDRL3 | sequence of CDRL3 | | |
| | (IMGT) | of 1D05 using IMGT | | |
| 40 | 1D05 - | Amino acid | | RASQSISSYLN |
| | CDRL1 | sequence of CDRL1 | | |
| | (Kabat) | of 1D05 using | | |
| | | Kabat | | |
| 41 | 1D05 - | Amino acid | | VASSLQS |
| | CDRL2 | sequence of CDRL2 | | |
| | (Kabat) | of 1D05 using | | |
| | | Kabat | | |
| 42 | 1D05 - | Amino acid | | QQSYSTPIT |
| | CDRL3 | sequence of CDRL3 | | |
| | (Kabat) | of 1D05 using | | |
| | | Kabat | | |
| 43 | 1D05 - | Amino acid | | DIQMTQSPSSLSASVGDRVTITCRASQSISSYLNWYQQKPGKAPKL |
| | Light | sequence of V_{L} of | | LIYVASSLQSGVPSRFSGSGSGTDFTLTISSLQPEDFATYYCQQSY |
| | chain | 1D05 (mutations | | STPITFGQGTRLEIK |
| | variable | from germline are | | |
| | region | shown in bold | | |
| | | letters) | | |
| 44 | 1D05 - | Nucleic acid | | AAAGCTTGCCGCCACCATGAGGCTCCCTGCTCAGCTTCTGGGGCTC |
| | Light | sequence of V_{L} of | | CTGCTACTCTGGCTCCGAGGTGCCAGATGTGACATCCAGATGACCC |
| | chain | 1D05 | | AGTCTCCATCCTCCCTGTCTGCATCTGTAGGAGACAGAGTCACCAT |
| | variable | | | CACTTGCCGGGCAAGTCAGAGCATTAGCAGCTATTTAAATTGGTAT |
| | region | | | CAGCAGAAACCAGGGAAAGCCCCTAAACTCCTGATCTATGTTGCAT |
| | | | | CCAGTTTGCAAAGTGGGGTCCCATCAAGGTTCAGTGGCAGTGGATC |
| | | | | TGGGACAGATTTCACTCTCACTATCAGCAGTCTGCAACCTGAAGAT |
| | | | | TTTGCAACTTACTACTGTCAACAGAGTTACAGTACCCCGATCACCT |
| | | | | TCGGCCAAGGGACACGTCTGGAGATCAAACGTACGGATGCTGCACC |
| | | | | AACT |
| 45 | 1D05 - | Amino acid | | DIQMTQSPSSLSASVGDRVTITCRASQSISSYLNWYQQKPGKAPKL |
| | full | sequence of 1D05 | | LIYVASSLQSGVPSRFSGSGSGTDFTLTISSLQPEDFATYYCQQSY |
| | light | light chain | | STPITFGQGTRLEIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 46 | 1D05 - | Nucleic acid | | GACATCCAGATGACCCAGTCCCCCTCCAGCCTGTCTGCTTCCGTGG |
| | full | sequence of 1D05 | | GCGACAGAGTGACCATCACCTGTCGGGCCTCCCAGTCCATCTCCTC |
| | light | light chain | | CTACCTGAACTGGTATCAGCAGAAGCCCGGCAAGGCCCCCAAGCTG |
| | chain | | | CTGATCTACGTGGCCAGCTCTCTGCAGTCCGGCGTGCCCTCTAGAT |
| | sequence | | | TCTCCGGCTCTGGCTCTGGCACCGACTTTACCCTGACCATCAGCTC |
| | | | | CCTGCAGCCCGAGGACTTCGCCACCTACTACTGCCAGCAGTCCTAC |
| | | | | TCCACCCCTATCACCTTCGGCCAGGGCACCCGGCTGGAAATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 47 | Mutated | Amino acid | | EVQLVESGGGLVQPGRSLRLSCAASGFTFDDYAMHWVRQAPGKGLE |
| | 1D05 - | sequence of 1D05 | | WVSGISWIRTGIGYADSVKGRFTIFRDNAKNSLYLQMNSLRAEDTA |
| | HC | heavy chain with V | | LYYCAKDMKGSGTYGGWFDTWGQGTLVTVSSASTKGPSVFPLAPCS |
| | mutant 1 | to A back-mutation | | RSTSESTAALGCLVKDYFPEPVTVSWNSGALTSGVHTFPAVLQSSG |
| | | in framework | | LYSLSSVVTVPSSSLGTKTYTCNVDHKPSNTKVDKRVESKYGPPCP |
| | | region to germline | | PCPAPELAGAPSVFLFPPKPKDTLMISRTPEVTCVVVDVSQEDPEV |
| | | highlighted with | | QFNWYVDGVEVHNAKTKPREEQFNSTYRVVSVLTVLHQDWLNGKEY |
| | | IgG1 disabled | | KCKVSNKGLPSSIEKTISKAKGQPREPQVYTLPPSQEEMTKNQVSL |
| | | (LAGA) constant | | TCLVKGFYPSDIAVEWESNGQPENNYKTTPPVLDSDGSFFLYSRLT |
| | | region | | VDKSRWQEGNVFSCSVMHEALHNHYTQKSLSLSLGK |
| 48 | Mutated | Amino acid | | EVQLVESGGGLVQPGRSLRLSCAASGFTFDDYAMHWVRQVPGKGLE |
| | 1D05 - | sequence of 1D05 | | WVSGISWIRTGIGYADSVKGRFTISRDNAKNSLYLQMNSLRAEDTA |
| | HC | heavy chain with F | | LYYCAKDMKGSGTYGGWFDTWGQGTLVTVSSASTKGPSVFPLAPCS |
| | mutant 2 | to S back-mutation | | RSTSESTAALGCLVKDYFPEPVTVSWNSGALTSGVHTFPAVLQSSG |
| | | in framework | | LYSLSSVVTVPSSSLGTKTYTCNVDHKPSNTKVDKRVESKYGPPCP |
| | | region to germline | | PCPAPELAGAPSVFLFPPKPKDTLMISRTPEVTCVVVDVSQEDPEV |
| | | highlighted with | | QFNWYVDGVEVHNAKTKPREEQFNSTYRVVSVLTVLHQDWLNGKEY |
| | | IgG1 disabled | | KCKVSNKGLPSSIEKTISKAKGQPREPQVYTLPPSQEEMTKNQVSL |
| | | (LAGA) constant | | TCLVKGFYPSDIAVEWESNGQPENNYKTTPPVLDSDGSFFLYSRLT |
| | | region | | VDKSRWQEGNVFSCSVMHEALHNHYTQKSLSLSLGK |
| 49 | Mutated | Amino acid | | EVQLVESGGGLVQPGRSLRLSCAASGFTFDDYAMHWVRQVPGKGLE |
| | 1D05 - | sequence of 1D05 | | WVSGISWIRTGIGYADSVKGRFTIFRDNAKNSLYLQMNSLRAEDTA |
| | HC | heavy chain with | | LYYCAKDMKGSGTYGGWFDTWGQGTLVTVSSASTKGPSVFPLAPCS |
| | mutant 3 | ELLG to -PVA back- | | RSTSESTAALGCLVKDYFPEPVTVSWNSGALTSGVHTFPAVLQSSG |
| | | mutation in | | LYSLSSVVTVPSSSLGTKTYTCNVDHKPSNTKVDKRVESKYGPPCP |
| | | constant region to | | PCPAP- |
| | | germline | | PVAGPSVFLFPPKPKDTLMISRTPEVTCVVVDVSQEDPEVQFNWYV |
| | | highlighted | | DGVEVHNAKTKPREEQFNSTYRVVSVLTVLHQDWLNGKEYKCKVSN |
| | | | | KGLPSSIEKTISKAKGQPREPQVYTLPPSQEEMTKNQVSLTCLVKG |
| | | | | FYPSDIAVEWESNGQPENNYKTTPPVLDSDGSFFLYSRLTVDKSRW |
| | | | | QEGNVFSCSVMHEALHNHYTQKSLSLSLGK |
| 50 | Mutated | Amino acid | | DIQMTQSPSSLSASVGDRVTITCRASQSISSYLNWYQQKPGKAPKL |
| | 1D05 - | sequence of 1D05 | | LIYAASSLQSGVPSRFSGSGSGTDFTLTISSLQPEDFATYYCQQSY |
| | LC | kappa light chain | | STPITFGQGTRLEIKRTVAAPSVFIFPPSDEQLKSGTASVVCLLNN |
| | mutant 1 | with V to A back- | | FYPREAKVQWKVDNALQSGNSQESVTEQDSKDSTYSLSSTLTLSKA |
| | | mutation in CDRL2 | | DYEKHKVYACEVTHQGLSSPVTKSFNRGEC |
| | | to germline | | |
| | | highlighted | | |
| 51 | Mutated | Amino acid | | DIQMTQSPSSLSASVGDRVTITCRASQSISSYLNWYQQKPGKAPKL |
| | 1D05 - | sequence of 1D05 | | FIYVASSLQSGVPSRFSGSGSGTDFTLTISSLQPEDFATYYCQQSY |
| | LC | kappa light chain | | STPITFGQGTRLEIKRTVAAPSVFIFPPSDEQLKSGTASVVCLLNN |
| | mutant 2 | with L to F back- | | FYPREAKVQWKVDNALQSGNSQESVTEQDSKDSTYSLSSTLTLSKA |
| | | mutation in | | DYEKHKVYACEVTHQGLSSPVTKSFNRGEC |
| | | framework to | | |
| | | germline | | |
| | | highlighted | | |
| 52 | 411B08 - | Amino acid | | GFTFSSYW |
| | CDRH1 | sequence of CDRH1 | | |
| | (IMGT) | of 411B08 using | | |
| | | IMGT | | |
| 53 | 411B08 - | Amino acid | | IKEDGSEK |
| | CDRH2 | sequence of CDRH2 | | |
| | (IMGT) | of 411B08 using | | |
| | | IMGT | | |
| 54 | 411B08 - | Amino acid | | ARNRLYSDFLDN |
| | CDRH3 | sequence of CDRH3 | | |
| | (IMGT) | of 411B08 using | | |
| | | IMGT | | |
| 55 | 411B08 - | Amino acid | | SYWMS |
| | CDRH1 | sequence of CDRH1 | | |
| | (Kabat) | of 411B08 using | | |
| | | Kabat | | |
| 56 | 411B08 - | Amino acid | | NIKEDGSEKYYVDSVKG |
| | CDRH2 | sequence of CDRH2 | | |
| | (Kabat) | of 411B08 using | | |
| | | Kabat | | |
| 57 | 411B08 - | Amino acid | | NRLYSDFLDN |
| | CDRH3 | sequence of CDRH3 | | |
| | (Kabat) | of 411B08 using | | |
| | | Kabat | | |
| 58 | 411B08 - | Amino acid | | EVQLVESGGGLVQPGGSLRLSCAASGFTFSSYWMSWVRQAPGKGLE |
| | Heavy | sequence of V_{H} of | | WVANIKEDGSEKYYVDSVKGRFTISRDNAKNSLYLQMNSLRAEDTS |
| | chain | 411B08 | | VYYCARNRLYSDFLDNWGQGTLVTVSS |
| | variable | | | |
| | region | | | |
| 59 | 411B08 - | Nucleic acid | | GAGGTGCAGCTGGTGGAGTCTGGGGGAGGCTTGGTCCAGCCTGGGG |
| | Heavy | sequence of V_{H} of | | GGTCCCTGAGACTCTCCTGTGCAGCCTCTGGATTCACGTTTAGTAG |
| | chain | 411B08 | | CTATTGGATGAGTTGGGTCCGCCAGGCTCCAGGGAAGGGGCTGGAG |
| | variable | | | TGGGTGGCCAACATCAAAGAAGATGGAAGTGAGAAATACTATGTCG |
| | region | | | ACTCTGTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAAATCGACTCTACAGTGACTTCCTTGACA |
| | | | | ACTGGGGCCAGGGAACCCTGGTCACCGTCTCCTCAG |
| 60 | 411B08 - | Amino acid | | EVQLVESGGGLVQPGGSLRLSCAASGFTFSSYWMSWVRQAPGKGLE |
| | full | sequence of 411B08 | | WVANIKEDGSEKYYVDSVKGRFTISRDNAKNSLYLQMNSLRAEDTS |
| | heavy | heavy chain | | VYYCARNRLYSDFLDNWGQGTLVTVSSASTKGPSVFPLAPSSKSTS |
| | chain | | | GGTAALGCLVKDYFPEPVTVSWNSGALTSGVHTFPAVLQSSGLYSL |
| | sequence | | | SSVVTVPSSSLGTQTYICNVNHKPSNTKVDKKVEPKSCDKTHTCPP |
| | | | | CPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYK |
| | | | | CKVSNKALPAPIEKTISKAKGQPREPQVYTLPPSRDELTKNQVSLT |
| | | | | CLVKGFYPSDIAVEWESNGQPENNYKTTPPVLDSDGSFFLYSKLTV |
| | | | | DKSRWQQGNVFSCSVMHEALHNHYTQKSLSLSPGK |
| 61 | 411B08 - | Nucleic acid | | GAGGTGCAGCTGGTGGAGTCTGGGGGAGGCTTGGTCCAGCCTGGGG |
| | full | sequence of 411B08 | | GGTCCCTGAGACTCTCCTGTGCAGCCTCTGGATTCACGTTTAGTAG |
| | heavy | heavy chain | | CTATTGGATGAGTTGGGTCCGCCAGGCTCCAGGGAAGGGGCTGGAG |
| | chain | | | TGGGTGGCCAACATCAAAGAAGATGGAAGTGAGAAATACTATGTCG |
| | sequence | | | ACTCTGTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAAATCGACTCTACAGTGACTTCCTTGACA |
| | | | | ACTGGGGCCAGGGAACCCTGGTCACCGTCTCCTCAGCCAGCACCAA |
| | | | | GGGCCCCTCTGTGTTCCCTCTGGCCCCTTCCAGCAAGTCCACCTCT |
| | | | | GGCGGAACAGCCGCTCTGGGCTGCCTCGTGAAGGACTACTTCCCCG |
| | | | | AGCCTGTGACCGTGTCCTGGAACTCTGGCGCTCTGACCAGCGGAGT |
| | | | | GCACACCTTCCCTGCTGTGCTGCAGTCCTCCGGCCTGTACTCCCTG |
| | | | | TCCTCCGTCGTGACCGTGCCTTCCAGCTCTCTGGGCACCCAGACCT |
| | | | | ACATCTGCAACGTGAACCACAAGCCCTCCAACACCAAGGTGGACAA |
| | | | | GAAGGTGGAACCCAAGTCCTGCGACAAGACCCACACCTGTCCCCCT |
| | | | | TGTCCTGCCCCTGAACTGCTGGGCGGACCTTCCGTGTTCCTGTTCC |
| | | | | CCCCAAAGCCCAAGGACACCCTGATGATCTCCCGGACCCCCGAAGT |
| | | | | GACCTGCGTGGTGGTGGATGTGTCCCACGAGGACCCTGAAGTGAAG |
| | | | | TTCAATTGGTACGTGGACGGCGTGGAAGTGCACAACGCCAAGACCA |
| | | | | AGCCTAGAGAGGAACAGTACAACTCCACCTACCGGGTGGTGTCCGT |
| | | | | GCTGACCGTGCTGCACCAGGATTGGCTGAACGGCAAAGAGTACAAG |
| | | | | TGCAAGGTGTCCAACAAGGCCCTGCCTGCCCCCATCGAAAAGACCA |
| | | | | TCTCCAAGGCCAAGGGCCAGCCCCGGGAACCCCAGGTGTACACACT |
| | | | | GCCCCCTAGCAGGGACGAGCTGACCAAGAACCAGGTGTCCCTGACC |
| | | | | TGTCTCGTGAAAGGCTTCTACCCCTCCGATATCGCCGTGGAATGGG |
| | | | | AGTCCAACGGCCAGCCTGAGAACAACTACAAGACCACCCCCCCTGT |
| | | | | GCTGGACTCCGACGGCTCATTCTTCCTGTACAGCAAGCTGACAGTG |
| | | | | GACAAGTCCCGGTGGCAGCAGGGCAACGTGTTCTCCTGCTCCGTGA |
| | | | | TGCACGAGGCCCTGCACAACCACTACACCCAGAAGTCCCTGTCCCT |
| | | | | GAGCCCCGGCAAG |
| 62 | 411B08 - | Amino acid | | QGVSSW |
| | CDRL1 | sequence of CDRL1 | | |
| | (IMGT) | of 411B08 using | | |
| | | IMGT | | |
| 63 | 411B08 - | Amino acid | | GAS |
| | CDRL2 | sequence of CDRL2 | | |
| | (IMGT) | of 411B08 using | | |
| | | IMGT | | |
| 64 | 411B08 - | Amino acid | | QQANSIPFT |
| | CDRL3 | sequence of CDRL3 | | |
| | (IMGT) | of 411B08 using | | |
| | | IMGT | | |
| 65 | 411B08 - | Amino acid | | RASQGVSSWLA |
| | CDRL1 | sequence of CDRL1 | | |
| | (Kabat) | of 411B08 using | | |
| | | Kabat | | |
| 66 | 411B08 - | Amino acid | | GASSLQS |
| | CDRL2 | sequence of CDRL2 | | |
| | (Kabat) | of 411B08 using | | |
| | | Kabat | | |
| 67 | 411B08 - | Amino acid | | QQANSIPFT |
| | CDRL3 | sequence of CDRL3 | | |
| | (Kabat) | of 411B08 using | | |
| | | Kabat | | |
| 68 | 411B08 - | Amino acid | | DIQMTQSPSSVSASVGDRVTITCRASQGVSSWLAWYQQKSGKAPKL |
| | Light | sequence of V_{L} of | | LIYGASSLQSGVPSRFSGSGSGTEFILTISSLQPEDFATYYCQQAN |
| | chain | 411B08 | | SIPFTFGPGTKVDIK |
| | variable | | | |
| | region | | | |
| 69 | 411B08 - | Nucleic acid | | GACATCCAGATGACCCAGTCTCCATCTTCCGTGTCTGCATCTGTCG |
| | Light | sequence of V_{L} of | | GAGACAGAGTCACCATCACTTGTCGGGCGAGTCAGGGTGTTAGCAG |
| | chain | 411B08 | | CTGGTTAGCCTGGTATCAGCAGAAATCAGGGAAAGCCCCTAAGCTC |
| | variable | | | CTGATCTATGGTGCATCCAGTTTGCAAAGTGGGGTCCCATCAAGAT |
| | region | | | TCAGCGGCAGTGGATCTGGGACAGAGTTCATTCTCACCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| 70 | 411B08 - | Amino acid | | DIQMTQSPSSVSASVGDRVTITCRASQGVSSWLAWYQQKSGKAPKL |
| | full | sequence of 411B08 | | LIYGASSLQSGVPSRFSGSGSGTEFILTISSLQPEDFATYYCQQAN |
| | light | light chain | | SIPFTFGPGTKVDIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 71 | 411B08 - | Nucleic acid | | GACATCCAGATGACCCAGTCTCCATCTTCCGTGTCTGCATCTGTCG |
| | full | sequence of 411B08 | | GAGACAGAGTCACCATCACTTGTCGGGCGAGTCAGGGTGTTAGCAG |
| | light | light chain | | CTGGTTAGCCTGGTATCAGCAGAAATCAGGGAAAGCCCCTAAGCTC |
| | chain | | | CTGATCTATGGTGCATCCAGTTTGCAAAGTGGGGTCCCATCAAGAT |
| | sequence | | | TCAGCGGCAGTGGATCTGGGACAGAGTTCATTCTCACCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 72 | 411C04 - | Amino acid | | GFTFSSYW |
| | CDRH1 | sequence of CDRH1 | | |
| | (IMGT) | of 411C04 using | | |
| | | IMGT | | |
| 73 | 411C04 - | Amino acid | | IKEDGSEK |
| | CDRH2 | sequence of CDRH2 | | |
| | (IMGT) | of 411C04 using | | |
| | | IMGT | | |
| 74 | 411C04 - | Amino acid | | ARVRLYSDFLDY |
| | CDRH3 | sequence of CDRH3 | | |
| | (IMGT) | of 411C04 using | | |
| | | IMGT | | |
| 75 | 411C04 - | Amino acid | | SYWMS |
| | CDRH1 | sequence of CDRH1 | | |
| | (Kabat) | of 411C04 using | | |
| | | Kabat | | |
| 76 | 411C04 - | Amino acid | | NIKEDGSEKYYVDSLKG |
| | CDRH2 | sequence of CDRH2 | | |
| | (Kabat) | of 411C04 using | | |
| | | Kabat | | |
| 77 | 411C04 - | Amino acid | | VRLYSDFLDY |
| | CDRH3 | sequence of CDRH3 | | |
| | (Kabat) | of 411C04 using | | |
| | | Kabat | | |
| 78 | 411C04 - | Amino acid | | EVQLVDSGGGLVQPGGSLRLSCAASGFTFSSYWMSWVRQAPGKGLE |
| | Heavy | sequence of V_{H} of | | WVANIKEDGSEKYYVDSLKGRFTISRDNAKNSLYLQMNSLRAEDTS |
| | chain | 411C04 | | VYYCARVRLYSDFLDYWGQGTLVTVSS |
| | variable | | | |
| | region | | | |
| 79 | 411C04 - | Nucleic acid | | GAGGTGCAGCTGGTGGACTCTGGGGGAGGCTTGGTCCAGCCTGGGG |
| | Heavy | sequence of V_{H} of | | GGTCCCTGAGACTCTCCTGTGCAGCCTCTGGATTCACGTTTAGTAG |
| | chain | 411C04 | | CTATTGGATGAGTTGGGTCCGCCAGGCTCCAGGAAAGGGGCTGGAG |
| | variable | | | TGGGTGGCCAACATAAAAGAAGATGGAAGTGAGAAATACTATGTAG |
| | region | | | ACTCTTTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAGTTCGACTCTACAGTGACTTCCTTGACT |
| | | | | ACTGGGGCCAGGGAACCCTGGTCACCGTCTCCTCAG |
| 80 | 411C04 - | Amino acid | | EVQLVDSGGGLVQPGGSLRLSCAASGFTFSSYWMSWVRQAPGKGLE |
| | full | sequence of 411C04 | | WVANIKEDGSEKYYVDSLKGRFTISRDNAKNSLYLQMNSLRAEDTS |
| | heavy | heavy chain | | VYYCARVRLYSDFLDYWGQGTLVTVSSASTKGPSVFPLAPSSKSTS |
| | chain | | | GGTAALGCLVKDYFPEPVTVSWNSGALTSGVHTFPAVLQSSGLYSL |
| | sequence | | | SSVVTVPSSSLGTQTYICNVNHKPSNTKVDKKVEPKSCDKTHTCPP |
| | | | | CPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYK |
| | | | | CKVSNKALPAPIEKTISKAKGQPREPQVYTLPPSRDELTKNQVSLT |
| | | | | CLVKGFYPSDIAVEWESNGQPENNYKTTPPVLDSDGSFFLYSKLTV |
| | | | | DKSRWQQGNVFSCSVMHEALHNHYTQKSLSLSPGK |
| 81 | 411C04 - | Nucleic acid | | GAGGTGCAGCTGGTGGACTCTGGGGGAGGCTTGGTCCAGCCTGGGG |
| | full | sequence of 411C04 | | GGTCCCTGAGACTCTCCTGTGCAGCCTCTGGATTCACGTTTAGTAG |
| | heavy | heavy chain | | CTATTGGATGAGTTGGGTCCGCCAGGCTCCAGGAAAGGGGCTGGAG |
| | chain | | | TGGGTGGCCAACATAAAAGAAGATGGAAGTGAGAAATACTATGTAG |
| | sequence | | | ACTCTTTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAGTTCGACTCTACAGTGACTTCCTTGACT |
| | | | | ACTGGGGCCAGGGAACCCTGGTCACCGTCTCCTCAGCCAGCACCAA |
| | | | | GGGCCCCTCTGTGTTCCCTCTGGCCCCTTCCAGCAAGTCCACCTCT |
| | | | | GGCGGAACAGCCGCTCTGGGCTGCCTCGTGAAGGACTACTTCCCCG |
| | | | | AGCCTGTGACCGTGTCCTGGAACTCTGGCGCTCTGACCAGCGGAGT |
| | | | | GCACACCTTCCCTGCTGTGCTGCAGTCCTCCGGCCTGTACTCCCTG |
| | | | | TCCTCCGTCGTGACCGTGCCTTCCAGCTCTCTGGGCACCCAGACCT |
| | | | | ACATCTGCAACGTGAACCACAAGCCCTCCAACACCAAGGTGGACAA |
| | | | | GAAGGTGGAACCCAAGTCCTGCGACAAGACCCACACCTGTCCCCCT |
| | | | | TGTCCTGCCCCTGAACTGCTGGGCGGACCTTCCGTGTTCCTGTTCC |
| | | | | CCCCAAAGCCCAAGGACACCCTGATGATCTCCCGGACCCCCGAAGT |
| | | | | GACCTGCGTGGTGGTGGATGTGTCCCACGAGGACCCTGAAGTGAAG |
| | | | | TTCAATTGGTACGTGGACGGCGTGGAAGTGCACAACGCCAAGACCA |
| | | | | AGCCTAGAGAGGAACAGTACAACTCCACCTACCGGGTGGTGTCCGT |
| | | | | GCTGACCGTGCTGCACCAGGATTGGCTGAACGGCAAAGAGTACAAG |
| | | | | TGCAAGGTGTCCAACAAGGCCCTGCCTGCCCCCATCGAAAAGACCA |
| | | | | TCTCCAAGGCCAAGGGCCAGCCCCGGGAACCCCAGGTGTACACACT |
| | | | | GCCCCCTAGCAGGGACGAGCTGACCAAGAACCAGGTGTCCCTGACC |
| | | | | TGTCTCGTGAAAGGCTTCTACCCCTCCGATATCGCCGTGGAATGGG |
| | | | | AGTCCAACGGCCAGCCTGAGAACAACTACAAGACCACCCCCCCTGT |
| | | | | GCTGGACTCCGACGGCTCATTCTTCCTGTACAGCAAGCTGACAGTG |
| | | | | GACAAGTCCCGGTGGCAGCAGGGCAACGTGTTCTCCTGCTCCGTGA |
| | | | | TGCACGAGGCCCTGCACAACCACTACACCCAGAAGTCCCTGTCCCT |
| | | | | GAGCCCCGGCAAG |
| 82 | 411C04 - | Amino acid | | QGVSSW |
| | CDRL1 | sequence of CDRL1 | | |
| | (IMGT) | of 411C04 using | | |
| | | IMGT | | |
| 83 | 411C04 - | Amino acid | | GAS |
| | CDRL2 | sequence of CDRL2 | | |
| | (IMGT) | of 411C04 using | | |
| | | IMGT | | |
| 84 | 411C04 - | Amino acid | | QQANSIPFT |
| | CDRL3 | sequence of CDRL3 | | |
| | (IMGT) | of 411C04 using | | |
| | | IMGT | | |
| 85 | 411C04 - | Amino acid | | RASQGVSSWLA |
| | CDRL1 | sequence of CDRL1 | | |
| | (Kabat) | of 411C04 using | | |
| | | Kabat | | |
| 86 | 411C04 - | Amino acid | | GASSLQS |
| | CDRL2 | sequence of CDRL2 | | |
| | (Kabat) | of 411C04 using | | |
| | | Kabat | | |
| 87 | 411C04 - | Amino acid | | QQANSIPFT |
| | CDRL3 | sequence of CDRL3 | | |
| | (Kabat) | of 411C04 using | | |
| | | Kabat | | |
| 88 | 411C04 - | Amino acid | | DIQMTQSPSSVSASVGDRVTITCRASQGVSSWLAWYQQKSGKAPKL |
| | Light | sequence of V_{L} of | | LIYGASSLQSGVPSRFSGSGSGTEFILSISSLQPEDFATYYCQQAN |
| | chain | 411C04 | | SIPFTFGPGTKVDIK |
| | variable | | | |
| | region | | | |
| 89 | 411C04 - | Nucleic acid | | GACATCCAGATGACCCAGTCTCCATCTTCCGTGTCTGCATCTGTCG |
| | Light | sequence of V_{L} of | | GAGACAGAGTCACCATCACTTGTCGGGCGAGTCAGGGTGTTAGCAG |
| | chain | 411C04 | | TTGGTTAGCCTGGTATCAGCAGAAATCAGGGAAAGCCCCTAAGCTC |
| | variable | | | CTGATCTATGGTGCCTCCAGTTTGCAAAGTGGGGTCCCATCAAGAT |
| | region | | | TCAGCGGCAGTGGATCTGGGACAGAGTTCATTCTCAGCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| 90 | 411C04 - | Amino acid | | DIQMTQSPSSVSASVGDRVTITCRASQGVSSWLAWYQQKSGKAPKL |
| | full | sequence of 411C04 | | LIYGASSLQSGVPSRFSGSGSGTEFILSISSLQPEDFATYYCQQAN |
| | light | light chain | | SIPFTFGPGTKVDIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 91 | 411C04 - | Nucleic acid | | GACATCCAGATGACCCAGTCTCCATCTTCCGTGTCTGCATCTGTCG |
| | full | sequence of 411C04 | | GAGACAGAGTCACCATCACTTGTCGGGCGAGTCAGGGTGTTAGCAG |
| | light | light chain | | TTGGTTAGCCTGGTATCAGCAGAAATCAGGGAAAGCCCCTAAGCTC |
| | chain | | | CTGATCTATGGTGCCTCCAGTTTGCAAAGTGGGGTCCCATCAAGAT |
| | sequence | | | TCAGCGGCAGTGGATCTGGGACAGAGTTCATTCTCAGCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 92 | 411D07 - | Amino acid | | GGSIISSDW |
| | CDRH1 | sequence of CDRH1 | | |
| | (IMGT) | of 411D07 using | | |
| | | IMGT | | |
| 93 | 411D07 - | Amino acid | | IFHSGRT |
| | CDRH2 | sequence of CDRH2 | | |
| | (IMGT) | of 411D07 using | | |
| | | IMGT | | |
| 94 | 411D07 - | Amino acid | | ARDGSGSY |
| | CDRH3 | sequence of CDRH3 | | |
| | (IMGT) | of 411D07 using | | |
| | | IMGT | | |
| 95 | 411D07 - | Amino acid | | SSDWWN |
| | CDRH1 | sequence of CDRH1 | | |
| | (Kabat) | of 411D07 using | | |
| | | Kabat | | |
| 96 | 411D07 - | Amino acid | | EIFHSGRTNYNPSLKS |
| | CDRH2 | sequence of CDRH2 | | |
| | (Kabat) | of 411D07 using | | |
| | | Kabat | | |
| 97 | 411D07 - | Amino acid | | DGSGSY |
| | CDRH3 | sequence of CDRH3 | | |
| | (Kabat) | of 411D07 using | | |
| | | Kabat | | |
| 98 | 411D07 - | Amino acid | | QVQLQESGPGLVKPSGTLSLTCIVSGGSIISSDWWNWVRQPPGKGL |
| | Heavy | sequence of V_{H} of | | EWIGEIFHSGRTNYNPSLKSRVTISIDKSKNQFSLRLSSVTAADTA |
| | chain | 411D07 | | VYYCARDGSGSYWGQGTLVTVSS |
| | variable | | | |
| | region | | | |
| 99 | 411D07 - | Nucleic acid | | CAGGTGCAGCTGCAGGAGTCGGGCCCAGGACTGGTGAAGCCTTCGG |
| | Heavy | sequence of V_{H} of | | GGACCCTGTCCCTCACCTGCATTGTCTCTGGTGGCTCCATCATCAG |
| | chain | 411D07 | | TAGTGACTGGTGGAATTGGGTCCGCCAGCCCCCAGGGAAGGGGCTG |
| | variable | | | GAGTGGATTGGAGAAATCTTTCATAGTGGGAGGACCAACTACAACC |
| | region | | | CGTCCCTCAAGAGTCGAGTCACCATATCAATAGACAAGTCCAAGAA |
| | | | | TCAGTTCTCCCTGAGGCTGAGCTCTGTGACCGCCGCGGACACGGCC |
| | | | | GTGTATTACTGTGCGAGAGATGGTTCGGGGAGTTACTGGGGCCAGG |
| | | | | GAACCCTGGTCACCGTCTCCTCAG |
| 100 | 411D07 - | Amino acid | | QVQLQESGPGLVKPSGTLSLTCIVSGGSIISSDWWNWVRQPPGKGL |
| | full | sequence of 411D07 | | EWIGEIFHSGRTNYNPSLKSRVTISIDKSKNQFSLRLSSVTAADTA |
| | heavy | heavy chain | | VYYCARDGSGSYWGQGTLVTVSSASTKGPSVFPLAPSSKSTSGGTA |
| | chain | | | ALGCLVKDYFPEPVTVSWNSGALTSGVHTFPAVLQSSGLYSLSSVV |
| | sequence | | | TVPSSSLGTQTYICNVNHKPSNTKVDKKVEPKSCDKTHTCPPCPAP |
| | | | | ELLGGPSVFLFPPKPKDTLMISRTPEVTCVVVDVSHEDPEVKFNWY |
| | | | | VDGVEVHNAKTKPREEQYNSTYRVVSVLTVLHQDWLNGKEYKCKVS |
| | | | | NKALPAPIEKTISKAKGQPREPQVYTLPPSRDELTKNQVSLTCLVK |
| | | | | GFYPSDIAVEWESNGQPENNYKTTPPVLDSDGSFFLYSKLTVDKSR |
| | | | | WQQGNVFSCSVMHEALHNHYTQKSLSLSPGK |
| 101 | 411D07 - | Nucleic acid | | CAGGTGCAGCTGCAGGAGTCGGGCCCAGGACTGGTGAAGCCTTCGG |
| | full | sequence of 411D07 | | GGACCCTGTCCCTCACCTGCATTGTCTCTGGTGGCTCCATCATCAG |
| | heavy | heavy chain | | TAGTGACTGGTGGAATTGGGTCCGCCAGCCCCCAGGGAAGGGGCTG |
| | chain | | | GAGTGGATTGGAGAAATCTTTCATAGTGGGAGGACCAACTACAACC |
| | sequence | | | CGTCCCTCAAGAGTCGAGTCACCATATCAATAGACAAGTCCAAGAA |
| | | | | TCAGTTCTCCCTGAGGCTGAGCTCTGTGACCGCCGCGGACACGGCC |
| | | | | GTGTATTACTGTGCGAGAGATGGTTCGGGGAGTTACTGGGGCCAGG |
| | | | | GAACCCTGGTCACCGTCTCCTCAGCCAGCACCAAGGGCCCCTCTGT |
| | | | | GTTCCCTCTGGCCCCTTCCAGCAAGTCCACCTCTGGCGGAACAGCC |
| | | | | GCTCTGGGCTGCCTCGTGAAGGACTACTTCCCCGAGCCTGTGACCG |
| | | | | TGTCCTGGAACTCTGGCGCTCTGACCAGCGGAGTGCACACCTTCCC |
| | | | | TGCTGTGCTGCAGTCCTCCGGCCTGTACTCCCTGTCCTCCGTCGTG |
| | | | | ACCGTGCCTTCCAGCTCTCTGGGCACCCAGACCTACATCTGCAACG |
| | | | | TGAACCACAAGCCCTCCAACACCAAGGTGGACAAGAAGGTGGAACC |
| | | | | CAAGTCCTGCGACAAGACCCACACCTGTCCCCCTTGTCCTGCCCCT |
| | | | | GAACTGCTGGGCGGACCTTCCGTGTTCCTGTTCCCCCCAAAGCCCA |
| | | | | AGGACACCCTGATGATCTCCCGGACCCCCGAAGTGACCTGCGTGGT |
| | | | | GGTGGATGTGTCCCACGAGGACCCTGAAGTGAAGTTCAATTGGTAC |
| | | | | GTGGACGGCGTGGAAGTGCACAACGCCAAGACCAAGCCTAGAGAGG |
| | | | | AACAGTACAACTCCACCTACCGGGTGGTGTCCGTGCTGACCGTGCT |
| | | | | GCACCAGGATTGGCTGAACGGCAAAGAGTACAAGTGCAAGGTGTCC |
| | | | | AACAAGGCCCTGCCTGCCCCCATCGAAAAGACCATCTCCAAGGCCA |
| | | | | AGGGCCAGCCCCGGGAACCCCAGGTGTACACACTGCCCCCTAGCAG |
| | | | | GGACGAGCTGACCAAGAACCAGGTGTCCCTGACCTGTCTCGTGAAA |
| | | | | GGCTTCTACCCCTCCGATATCGCCGTGGAATGGGAGTCCAACGGCC |
| | | | | AGCCTGAGAACAACTACAAGACCACCCCCCCTGTGCTGGACTCCGA |
| | | | | CGGCTCATTCTTCCTGTACAGCAAGCTGACAGTGGACAAGTCCCGG |
| | | | | TGGCAGCAGGGCAACGTGTTCTCCTGCTCCGTGATGCACGAGGCCC |
| | | | | TGCACAACCACTACACCCAGAAGTCCCTGTCCCTGAGCCCCGGCAA |
| | | | | G |
| 102 | 411D07 - | Amino acid | | QSVLYSSNNKNY |
| | CDRL1 | sequence of CDRL1 | | |
| | (IMGT) | of 411D07 using | | |
| | | IMGT | | |
| 103 | 411D07 - | Amino acid | | WAS |
| | CDRL2 | sequence of CDRL2 | | |
| | (IMGT) | of 411D07 using | | |
| | | IMGT | | |
| 104 | 411D07 - | Amino acid | | QQYYSNRS |
| | CDRL3 | sequence of CDRL3 | | |
| | (IMGT) | of 411D07 using | | |
| | | IMGT | | |
| 105 | 411D07 - | Amino acid | | KSSQSVLYSSNNKNYLA |
| | CDRL1 | sequence of CDRL1 | | |
| | (Kabat) | of 411D07 using | | |
| | | Kabat | | |
| 106 | 411D07 - | Amino acid | | WASTRES |
| | CDRL2 | sequence of CDRL2 | | |
| | (Kabat) | of 411D07 using | | |
| | | Kabat | | |
| 107 | 411D07 - | Amino acid | | QQYYSNRS |
| | CDRL3 | sequence of CDRL3 | | |
| | (Kabat) | of 411D07 using | | |
| | | Kabat | | |
| 108 | 411D07 - | Amino acid | | DIVMTQSPDSLAVSLGERATINCKSSQSVLYSSNNKNYLAWYQQKS |
| | Light | sequence of V_{L} of | | GQPPKLLIYWASTRESGVPDRFSGSGSGTDFTLTISSLQTEDVAVY |
| | chain | 411D07 | | YCQQYYSNRSFGQGTKLEIK |
| | variable | | | |
| | region | | | |
| 109 | 411D07 - | Nucleic acid | | GACATCGTGATGACCCAGTCTCCAGACTCCCTGGCTGTGTCTCTGG |
| | Light | sequence of V_{L} of | | GCGAGAGGGCCACCATCAACTGCAAGTCCAGCCAGAGTGTTTTATA |
| | chain | 411D07 | | CAGCTCCAACAATAAGAATTACTTAGCTTGGTACCAGCAGAAATCA |
| | variable | | | GGACAGCCTCCTAAGTTGCTCATTTACTGGGCATCTACCCGGGAAT |
| | region | | | CCGGGGTCCCTGACCGATTCAGTGGCAGCGGGTCTGGGACAGATTT |
| | | | | CACTCTCACCATCAGCAGCCTGCAGACTGAAGATGTGGCAGTTTAT |
| | | | | TACTGTCAGCAATATTATAGTAATCGCAGTTTTGGCCAGGGGACCA |
| | | | | AGCTGGAGATCAAAC |
| 110 | 411D07 - | Amino acid | | DIVMTQSPDSLAVSLGERATINCKSSQSVLYSSNNKNYLAWYQQKS |
| | full | sequence of 411D07 | | GQPPKLLIYWASTRESGVPDRFSGSGSGTDFTLTISSLQTEDVAVY |
| | light | light chain | | YCQQYYSNRSFGQGTKLEIKRTVAAPSVFIFPPSDEQLKSGTASVV |
| | chain | | | CLLNNFYPREAKVQWKVDNALQSGNSQESVTEQDSKDSTYSLSSTL |
| | sequence | | | TLSKADYEKHKVYACEVTHQGLSSPVTKSFNRGEC |
| 111 | 411D07 - | Nucleic acid | | GACATCGTGATGACCCAGTCTCCAGACTCCCTGGCTGTGTCTCTGG |
| | full | sequence of 411D07 | | GCGAGAGGGCCACCATCAACTGCAAGTCCAGCCAGAGTGTTTTATA |
| | light | light chain | | CAGCTCCAACAATAAGAATTACTTAGCTTGGTACCAGCAGAAATCA |
| | chain | | | GGACAGCCTCCTAAGTTGCTCATTTACTGGGCATCTACCCGGGAAT |
| | sequence | | | CCGGGGTCCCTGACCGATTCAGTGGCAGCGGGTCTGGGACAGATTT |
| | | | | CACTCTCACCATCAGCAGCCTGCAGACTGAAGATGTGGCAGTTTAT |
| | | | | TACTGTCAGCAATATTATAGTAATCGCAGTTTTGGCCAGGGGACCA |
| | | | | AGCTGGAGATCAAACGTACGGTGGCCGCTCCCTCCGTGTTCATCTT |
| | | | | CCCACCTTCCGACGAGCAGCTGAAGTCCGGCACCGCTTCTGTCGTG |
| | | | | TGCCTGCTGAACAACTTCTACCCCCGCGAGGCCAAGGTGCAGTGGA |
| | | | | AGGTGGACAACGCCCTGCAGTCCGGCAACTCCCAGGAATCCGTGAC |
| | | | | CGAGCAGGACTCCAAGGACAGCACCTACTCCCTGTCCTCCACCCTG |
| | | | | ACCCTGTCCAAGGCCGACTACGAGAAGCACAAGGTGTACGCCTGCG |
| | | | | AAGTGACCCACCAGGGCCTGTCTAGCCCCGTGACCAAGTCTTTCAA |
| | | | | CCGGGGCGAGTGT |
| 112 | 385F01 - | Amino acid | | GFTFSSYW |
| | CDRH1 | sequence of CDRH1 | | |
| | (IMGT) | of 385F01 using | | |
| | | IMGT | | |
| 113 | 385F01 - | Amino acid | | IKEDGSEK |
| | CDRH2 | sequence of CDRH2 | | |
| | (IMGT) | of 385F01 using | | |
| | | IMGT | | |
| 114 | 385F01 - | Amino acid | | ARNRLYSDFLDN |
| | CDRH3 | sequence of CDRH3 | | |
| | (IMGT) | of 385F01 using | | |
| | | IMGT | | |
| 115 | 385F01 - | Amino acid | | SYWMS |
| | CDRH1 | sequence of CDRH1 | | |
| | (Kabat) | of 385F01 using | | |
| | | Kabat | | |
| 116 | 385F01 - | Amino acid | | NIKEDGSEKYYVDSVKG |
| | CDRH2 | sequence of CDRH2 | | |
| | (Kabat) | of 385F01 using | | |
| | | Kabat | | |
| 117 | 385F01 - | Amino acid | | NRLYSDFLDN |
| | CDRH3 | sequence of CDRH3 | | |
| | (Kabat) | of 385F01 using | | |
| | | Kabat | | |
| 118 | 385F01 - | Amino acid | | EVQLVESGGGLVQPGGSLRLSCAASGFTFSSYWMSWVRQAPGKGLE |
| | Heavy | sequence of V_{H} of | | WVANIKEDGSEKYYVDSVKGRFTISRDNAKNSLYLQMNSLRAEDTS |
| | chain | 385F01 | | VYYCARNRLYSDFLDNWGQGTLVTVSS |
| | variable | | | |
| | region | | | |
| 119 | 385F01 - | Nucleic acid | | GAGGTGCAGCTGGTGGAGTCTGGGGGAGGCTTGGTCCAGCCTGGGG |
| | Heavy | sequence of V_{H} of | | GGTCCCTGAGACTCTCCTGTGCAGCCTCTGGATTCACGTTTAGTAG |
| | chain | 385F01 | | CTATTGGATGAGTTGGGTCCGCCAGGCTCCAGGGAAGGGGCTGGAG |
| | variable | | | TGGGTGGCCAACATCAAAGAAGATGGAAGTGAGAAATACTATGTCG |
| | region | | | ACTCTGTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAAATCGACTCTACAGTGACTTCCTTGACA |
| | | | | ACTGGGGCCAGGGAACCCTGGTCACCGTCTCCTCAG |
| 120 | 385F01 - | Amino acid | | EVQLVESGGGLVQPGGSLRLSCAASGFTFSSYWMSWVRQAPGKGLE |
| | full | sequence of 385F01 | | WVANIKEDGSEKYYVDSVKGRFTISRDNAKNSLYLQMNSLRAEDTS |
| | heavy | heavy chain | | VYYCARNRLYSDFLDNWGQGTLVTVSSASTKGPSVFPLAPSSKSTS |
| | chain | | | GGTAALGCLVKDYFPEPVTVSWNSGALTSGVHTFPAVLQSSGLYSL |
| | sequence | | | SSVVTVPSSSLGTQTYICNVNHKPSNTKVDKKVEPKSCDKTHTCPP |
| | | | | CPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYK |
| | | | | CKVSNKALPAPIEKTISKAKGQPREPQVYTLPPSRDELTKNQVSLT |
| | | | | CLVKGFYPSDIAVEWESNGQPENNYKTTPPVLDSDGSFFLYSKLTV |
| | | | | DKSRWQQGNVFSCSVMHEALHNHYTQKSLSLSPGK |
| 121 | 385F01 - | Nucleic acid | | GAGGTGCAGCTGGTGGAGTCTGGGGGAGGCTTGGTCCAGCCTGGGG |
| | full | sequence of 385F01 | | GGTCCCTGAGACTCTCCTGTGCAGCCTCTGGATTCACGTTTAGTAG |
| | heavy | heavy chain | | CTATTGGATGAGTTGGGTCCGCCAGGCTCCAGGGAAGGGGCTGGAG |
| | chain | | | TGGGTGGCCAACATCAAAGAAGATGGAAGTGAGAAATACTATGTCG |
| | sequence | | | ACTCTGTGAAGGGCCGATTCACCATCTCCAGAGACAACGCCAAGAA |
| | | | | CTCACTGTATCTGCAAATGAACAGCCTGAGAGCCGAGGACACGTCT |
| | | | | GTGTATTACTGTGCGAGAAATCGACTCTACAGTGACTTCCTTGACA |
| | | | | ACTGGGGCCAGGGAACCCTGGTCACCGTCTCCTCAGCCAGCACCAA |
| | | | | GGGCCCCTCTGTGTTCCCTCTGGCCCCTTCCAGCAAGTCCACCTCT |
| | | | | GGCGGAACAGCCGCTCTGGGCTGCCTCGTGAAGGACTACTTCCCCG |
| | | | | AGCCTGTGACCGTGTCCTGGAACTCTGGCGCTCTGACCAGCGGAGT |
| | | | | GCACACCTTCCCTGCTGTGCTGCAGTCCTCCGGCCTGTACTCCCTG |
| | | | | TCCTCCGTCGTGACCGTGCCTTCCAGCTCTCTGGGCACCCAGACCT |
| | | | | ACATCTGCAACGTGAACCACAAGCCCTCCAACACCAAGGTGGACAA |
| | | | | GAAGGTGGAACCCAAGTCCTGCGACAAGACCCACACCTGTCCCCCT |
| | | | | TGTCCTGCCCCTGAACTGCTGGGCGGACCTTCCGTGTTCCTGTTCC |
| | | | | CCCCAAAGCCCAAGGACACCCTGATGATCTCCCGGACCCCCGAAGT |
| | | | | GACCTGCGTGGTGGTGGATGTGTCCCACGAGGACCCTGAAGTGAAG |
| | | | | TTCAATTGGTACGTGGACGGCGTGGAAGTGCACAACGCCAAGACCA |
| | | | | AGCCTAGAGAGGAACAGTACAACTCCACCTACCGGGTGGTGTCCGT |
| | | | | GCTGACCGTGCTGCACCAGGATTGGCTGAACGGCAAAGAGTACAAG |
| | | | | TGCAAGGTGTCCAACAAGGCCCTGCCTGCCCCCATCGAAAAGACCA |
| | | | | TCTCCAAGGCCAAGGGCCAGCCCCGGGAACCCCAGGTGTACACACT |
| | | | | GCCCCCTAGCAGGGACGAGCTGACCAAGAACCAGGTGTCCCTGACC |
| | | | | TGTCTCGTGAAAGGCTTCTACCCCTCCGATATCGCCGTGGAATGGG |
| | | | | AGTCCAACGGCCAGCCTGAGAACAACTACAAGACCACCCCCCCTGT |
| | | | | GCTGGACTCCGACGGCTCATTCTTCCTGTACAGCAAGCTGACAGTG |
| | | | | GACAAGTCCCGGTGGCAGCAGGGCAACGTGTTCTCCTGCTCCGTGA |
| | | | | TGCACGAGGCCCTGCACAACCACTACACCCAGAAGTCCCTGTCCCT |
| | | | | GAGCCCCGGCAAG |
| 122 | 385F01 - | Amino acid | | QGVSSW |
| | CDRL1 | sequence of CDRL1 | | |
| | (IMGT) | of 385F01 using | | |
| | | IMGT | | |
| 123 | 385F01 - | Amino acid | | GAS |
| | CDRL2 | sequence of CDRL2 | | |
| | (IMGT) | of 385F01 using | | |
| | | IMGT | | |
| 124 | 385F01 - | Amino acid | | QQANSIPFT |
| | CDRL3 | sequence of CDRL3 | | |
| | (IMGT) | of 385F01 using | | |
| | | IMGT | | |
| 125 | 385F01 - | Amino acid | | RASQGVSSWLA |
| | CDRL1 | sequence of CDRL1 | | |
| | (Kabat) | of 385F01 using | | |
| | | Kabat | | |
| 126 | 385F01 - | Amino acid | | GASSLQS |
| | CDRL2 | sequence of CDRL2 | | |
| | (Kabat) | of 385F01 using | | |
| | | Kabat | | |
| 127 | 385F01 - | Amino acid | | QQANSIPFT |
| | CDRL3 | sequence of CDRL3 | | |
| | (Kabat) | of 385F01 using | | |
| | | Kabat | | |
| 128 | 385F01 - | Amino acid | | DIQMTQSPSSVSASVGDRVTITCRASQGVSSWLAWYQQKSGKAPKL |
| | Light | sequence of V_{L} of | | LIYGASSLQSGVPSRFSGSGSGTEFILTISSLQPEDFATYYCQQAN |
| | chain | 385F01 | | SIPFTFGPGTKVDIK |
| | variable | | | |
| | region | | | |
| 129 | 385F01 - | Nucleic acid | | GACATCCAGATGACCCAGTCTCCATCTTCCGTGTCTGCATCTGTCG |
| | Light | sequence of V_{L} of | | GAGACAGAGTCACCATCACTTGTCGGGCGAGTCAGGGTGTTAGCAG |
| | chain | 385F01 | | CTGGTTAGCCTGGTATCAGCAGAAATCAGGGAAAGCCCCTAAGCTC |
| | variable | | | CTGATCTATGGTGCATCCAGTTTGCAAAGTGGGGTCCCATCAAGAT |
| | region | | | TCAGCGGCAGTGGATCTGGGACAGAGTTCATTCTCACCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| 130 | 385F01 - | Amino acid | | DIQMTQSPSSVSASVGDRVTITCRASQGVSSWLAWYQQKSGKAPKL |
| | full | sequence of 385F01 | | LIYGASSLQSGVPSRFSGSGSGTEFILTISSLQPEDFATYYCQQAN |
| | light | light chain | | SIPFTFGPGTKVDIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 131 | 385F01 - | Nucleic acid | | GACATCCAGATGACCCAGTCTCCATCTTCCGTGTCTGCATCTGTCG |
| | full | sequence of 385F01 | | GAGACAGAGTCACCATCACTTGTCGGGCGAGTCAGGGTGTTAGCAG |
| | light | light chain | | CTGGTTAGCCTGGTATCAGCAGAAATCAGGGAAAGCCCCTAAGCTC |
| | chain | | | CTGATCTATGGTGCATCCAGTTTGCAAAGTGGGGTCCCATCAAGAT |
| | sequence | | | TCAGCGGCAGTGGATCTGGGACAGAGTTCATTCTCACCATCAGCAG |
| | | | | CCTGCAGCCTGAAGATTTTGCAACTTACTATTGTCAACAGGCTAAC |
| | | | | AGTATCCCATTCACTTTCGGCCCTGGGACCAAAGTGGATATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 132 | 413D08 - | Amino acid | | GFTFRIYG |
| | CDRH1 | sequence of CDRH1 | | |
| | (IMGT) | of 413D08 using | | |
| | | IMGT | | |
| 133 | 413D08 - | Amino acid | | IWYDGSNK |
| | CDRH2 | sequence of CDRH2 | | |
| | (IMGT) | of 413D08 using | | |
| | | IMGT | | |
| 134 | 413D08 - | Amino acid | | ARDMDYFGMDV |
| | CDRH3 | sequence of CDRH3 | | |
| | (IMGT) | of 413D08 using | | |
| | | IMGT | | |
| 135 | 413D08 - | Amino acid | | IYGMH |
| | CDRH1 | sequence of CDRH1 | | |
| | (Kabat) | of 413D08 using | | |
| | | Kabat | | |
| 136 | 413D08 - | Amino acid | | VIWYDGSNKYYADSVKG |
| | CDRH2 | sequence of CDRH2 | | |
| | (Kabat) | of 413D08 using | | |
| | | Kabat | | |
| 137 | 413D08 - | Amino acid | | DMDYFGMDV |
| | CDRH3 | sequence of CDRH3 | | |
| | (Kabat) | of 413D08 using | | |
| | | Kabat | | |
| 138 | 413D08 - | Amino acid | | QVQLVESGGGVVQPGRSLRLSCAASGFTFRIYGMHWVRQAPGKGLE |
| | Heavy | sequence of V_{H} of | | WVAVIWYDGSNKYYADSVKGRFTISRDNSDNTLYLQMNSLRAEDTA |
| | chain | 413D08 | | VYYCARDMDYFGMDVWGQGTTVTVSS |
| | variable | | | |
| | region | | | |
| 139 | 413D08 - | Nucleic acid | | CAGGTGCAGCTGGTGGAGTCTGGGGGAGGCGTGGTCCAGCCTGGGA |
| | Heavy | sequence of V_{H} of | | GGTCCCTGAGACTCTCCTGTGCAGCGTCTGGATTCACCTTCCGTAT |
| | chain | 413D08 | | TTATGGCATGCACTGGGTCCGCCAGGCTCCAGGCAAGGGGCTGGAG |
| | variable | | | TGGGTGGCAGTTATATGGTATGATGGAAGTAATAAATACTATGCTG |
| | region | | | ACTCCGTGAAGGGCCGATTCACCATCTCCAGAGACAATTCCGACAA |
| | | | | CACGCTGTATCTGCAAATGAACAGCCTGAGAGCCGAGGACACGGCT |
| | | | | GTGTATTACTGTGCGAGAGATATGGACTACTTCGGTATGGACGTCT |
| | | | | GGGGCCAAGGGACCACGGTCACCGTCTCCTCAG |
| 140 | 413D08 - | Amino acid | | QVQLVESGGGVVQPGRSLRLSCAASGFTFRIYGMHWVRQAPGKGLE |
| | full | sequence of 413D08 | | WVAVIWYDGSNKYYADSVKGRFTISRDNSDNTLYLQMNSLRAEDTA |
| | heavy | heavy chain | | VYYCARDMDYFGMDVWGQGTTVTVSSASTKGPSVFPLAPSSKSTSG |
| | chain | | | GTAALGCLVKDYFPEPVTVSWNSGALTSGVHTFPAVLQSSGLYSLS |
| | sequence | | | SVVTVPSSSLGTQTYICNVNHKPSNTKVDKKVEPKSCDKTHTCPPC |
| | | | | RAPELLGGPSVFLFPPKPKDTLMISRTPEVTCVVVDVSHEDPEVKF |
| | | | | NWYVDGVEVHNAKTKPREEQYNSTYRVVSVLTVLHQDWLNGKEYKC |
| | | | | KVSNKALPAPIEKTISKAKGQPREPQVYTLPPSRDELTKNQVSLTC |
| | | | | LVKGFYPSDIAVEWESNGQPENNYKTTPPVLDSDGSFFLYSKLTVD |
| | | | | KSRWQQGNVFSCSVMHEALHNHYTQKSLSLSPGK |
| 141 | 413D08 - | Nucleic acid | | CAGGTGCAGCTGGTGGAGTCTGGGGGAGGCGTGGTCCAGCCTGGGA |
| | full | sequence of 413D08 | | GGTCCCTGAGACTCTCCTGTGCAGCGTCTGGATTCACCTTCCGTAT |
| | heavy | heavy chain | | TTATGGCATGCACTGGGTCCGCCAGGCTCCAGGCAAGGGGCTGGAG |
| | chain | | | TGGGTGGCAGTTATATGGTATGATGGAAGTAATAAATACTATGCTG |
| | sequence | | | ACTCCGTGAAGGGCCGATTCACCATCTCCAGAGACAATTCCGACAA |
| | | | | CACGCTGTATCTGCAAATGAACAGCCTGAGAGCCGAGGACACGGCT |
| | | | | GTGTATTACTGTGCGAGAGATATGGACTACTTCGGTATGGACGTCT |
| | | | | GGGGCCAAGGGACCACGGTCACCGTCTCCTCAGCCAGCACCAAGGG |
| | | | | CCCCTCTGTGTTCCCTCTGGCCCCTTCCAGCAAGTCCACCTCTGGC |
| | | | | GGAACAGCCGCTCTGGGCTGCCTCGTGAAGGACTACTTCCCCGAGC |
| | | | | CTGTGACCGTGTCCTGGAACTCTGGCGCTCTGACCAGCGGAGTGCA |
| | | | | CACCTTCCCTGCTGTGCTGCAGTCCTCCGGCCTGTACTCCCTGTCC |
| | | | | TCCGTCGTGACCGTGCCTTCCAGCTCTCTGGGCACCCAGACCTACA |
| | | | | TCTGCAACGTGAACCACAAGCCCTCCAACACCAAGGTGGACAAGAA |
| | | | | GGTGGAACCCAAGTCCTGCGACAAGACCCACACCTGTCCCCCTTGT |
| | | | | CCTGCCCCTGAACTGCTGGGCGGACCTTCCGTGTTCCTGTTCCCCC |
| | | | | CAAAGCCCAAGGACACCCTGATGATCTCCCGGACCCCCGAAGTGAC |
| | | | | CTGCGTGGTGGTGGATGTGTCCCACGAGGACCCTGAAGTGAAGTTC |
| | | | | AATTGGTACGTGGACGGCGTGGAAGTGCACAACGCCAAGACCAAGC |
| | | | | CTAGAGAGGAACAGTACAACTCCACCTACCGGGTGGTGTCCGTGCT |
| | | | | GACCGTGCTGCACCAGGATTGGCTGAACGGCAAAGAGTACAAGTGC |
| | | | | AAGGTGTCCAACAAGGCCCTGCCTGCCCCCATCGAAAAGACCATCT |
| | | | | CCAAGGCCAAGGGCCAGCCCCGGGAACCCCAGGTGTACACACTGCC |
| | | | | CCCTAGCAGGGACGAGCTGACCAAGAACCAGGTGTCCCTGACCTGT |
| | | | | CTCGTGAAAGGCTTCTACCCCTCCGATATCGCCGTGGAATGGGAGT |
| | | | | CCAACGGCCAGCCTGAGAACAACTACAAGACCACCCCCCCTGTGCT |
| | | | | GGACTCCGACGGCTCATTCTTCCTGTACAGCAAGCTGACAGTGGAC |
| | | | | AAGTCCCGGTGGCAGCAGGGCAACGTGTTCTCCTGCTCCGTGATGC |
| | | | | ACGAGGCCCTGCACAACCACTACACCCAGAAGTCCCTGTCCCTGAG |
| | | | | CCCCGGCAAG |
| 142 | 413D08 - | Amino acid | | QGIRND |
| | CDRL1 | sequence of CDRL1 | | |
| | (IMGT) | of 413D08 using | | |
| | | IMGT | | |
| 143 | 413D08 - | Amino acid | | AAS |
| | CDRL2 | sequence of CDRL2 | | |
| | (IMGT) | of 413D08 using | | |
| | | IMGT | | |
| 144 | 413D08 - | Amino acid | | LQHNSYPRT |
| | CDRL3 | sequence of CDRL3 | | |
| | (IMGT) | of 413D08 using | | |
| | | IMGT | | |
| 145 | 413D08 - | Amino acid | | RASQGIRNDLG |
| | CDRL1 | sequence of CDRL1 | | |
| | (Kabat) | of 413D08 using | | |
| | | Kabat | | |
| 146 | 413D08 - | Amino acid | | AASSLQS |
| | CDRL2 | sequence of CDRL2 | | |
| | (Kabat) | of 413D08 using | | |
| | | Kabat | | |
| 147 | 413D08 - | Amino acid | | LQHNSYPRT |
| | CDRL3 | sequence of CDRL3 | | |
| | (Kabat) | of 413D08 using | | |
| | | Kabat | | |
| 148 | 413D08 - | Amino acid | | DLQMTQSPSSLSASVGDRVTITCRASQGIRNDLGWYQQKPGKAPKR |
| | Light | sequence of V_{L} of | | LIYAASSLQSGVPSRFSGSGSGTEFTLTISSLQPEDFATYYCLQHN |
| | chain | 413D08 | | SYPRTFGQGTKVEIK |
| | variable | | | |
| | region | | | |
| 149 | 413D08 - | Nucleic acid | | GACCTCCAGATGACCCAGTCTCCATCCTCCCTGTCTGCATCTGTAG |
| | Light | sequence of V_{L} of | | GAGACAGAGTCACCATCACTTGCCGGGCAAGTCAGGGCATTAGAAA |
| | chain | 413D08 | | TGATTTAGGCTGGTATCAGCAGAAACCAGGGAAAGCCCCTAAGCGC |
| | variable | | | CTGATCTATGCTGCATCCAGTTTGCAAAGTGGGGTCCCATCAAGGT |
| | region | | | TCAGCGGCAGTGGATCTGGGACAGAATTCACTCTCACAATCAGCAG |
| | | | | CCTGCAGCCTGAAGATTTTGCAACTTATTACTGTCTACAGCATAAT |
| | | | | AGTTACCCTCGGACGTTCGGCCAAGGGACCAAGGTGGAAATCAAAC |
| 150 | 413D08 - | Amino acid | | DLQMTQSPSSLSASVGDRVTITCRASQGIRNDLGWYQQKPGKAPKR |
| | full | sequence of 413D08 | | LIYAASSLQSGVPSRFSGSGSGTEFTLTISSLQPEDFATYYCLQHN |
| | light | light chain | | SYPRTFGQGTKVEIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 151 | 413D08 - | Nucleic acid | | GACCTCCAGATGACCCAGTCTCCATCCTCCCTGTCTGCATCTGTAG |
| | full | sequence of 413D08 | | GAGACAGAGTCACCATCACTTGCCGGGCAAGTCAGGGCATTAGAAA |
| | light | light chain | | TGATTTAGGCTGGTATCAGCAGAAACCAGGGAAAGCCCCTAAGCGC |
| | chain | | | CTGATCTATGCTGCATCCAGTTTGCAAAGTGGGGTCCCATCAAGGT |
| | sequence | | | TCAGCGGCAGTGGATCTGGGACAGAATTCACTCTCACAATCAGCAG |
| | | | | CCTGCAGCCTGAAGATTTTGCAACTTATTACTGTCTACAGCATAAT |
| | | | | AGTTACCCTCGGACGTTCGGCCAAGGGACCAAGGTGGAAATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 152 | 386H03 - | Amino acid | | GGSISSSDW |
| | CDRH1 | sequence of CDRH1 | | |
| | (IMGT) | of 386H03 using | | |
| | | IMGT | | |
| 153 | 386H03 - | Amino acid | | IFHSGNT |
| | CDRH2 | sequence of CDRH2 | | |
| | (IMGT) | of 386H03 using | | |
| | | IMGT | | |
| 154 | 386H03 - | Amino acid | | VRDGSGSY |
| | CDRH3 | sequence of CDRH3 | | |
| | (IMGT) | of 386H03 using | | |
| | | IMGT | | |
| 155 | 386H03 - | Amino acid | | SSDWWS |
| | CDRH1 | sequence of CDRH1 | | |
| | (Kabat) | of 386H03 using | | |
| | | Kabat | | |
| 156 | 386H03 - | Amino acid | | EIFHSGNTNYNPSLKS |
| | CDRH2 | sequence of CDRH2 | | |
| | (Kabat) | of 386H03 using | | |
| | | Kabat | | |
| 157 | 386H03 - | Amino acid | | DGSGSY |
| | CDRH3 | sequence of CDRH3 | | |
| | (Kabat) | of 386H03 using | | |
| | | Kabat | | |
| 158 | 386H03 - | Amino acid | | QVQLQESGPGLVKPSGTLSLTCAVSGGSISSSDWWSWVRQPPGKGL |
| | Heavy | sequence of V_{H} of | | EWIGEIFHSGNTNYNPSLKSRVTISVDKSKNQISLRLNSVTAADTA |
| | chain | 386H03 | | VYYCVRDGSGSYWGQGTLVTVSS |
| | variable | | | |
| | region | | | |
| 159 | 386H03 - | Nucleic acid | | CAGGTGCAGCTGCAGGAGTCGGGCCCAGGACTGGTGAAGCCTTCGG |
| | Heavy | sequence of V_{H} of | | GGACCCTGTCCCTCACCTGCGCTGTCTCTGGTGGCTCCATCAGCAG |
| | chain | 386H03 | | TAGTGACTGGTGGAGTTGGGTCCGCCAGCCCCCAGGGAAGGGGCTG |
| | variable | | | GAGTGGATTGGGGAAATCTTTCATAGTGGGAACACCAACTACAACC |
| | region | | | CGTCCCTCAAGAGTCGAGTCACCATATCAGTAGACAAGTCCAAGAA |
| | | | | CCAGATCTCCCTGAGGCTGAACTCTGTGACCGCCGCGGACACGGCC |
| | | | | GTGTATTACTGTGTGAGAGATGGTTCGGGGAGTTACTGGGGCCAGG |
| | | | | GAACCCTGGTCACCGTCTCCTCAG |
| 160 | 386H03 - | Amino acid | | QVQLQESGPGLVKPSGTLSLTCAVSGGSISSSDWWSWVRQPPGKGL |
| | full | sequence of 386H03 | | EWIGEIFHSGNTNYNPSLKSRVTISVDKSKNQISLRLNSVTAADTA |
| | heavy | heavy chain | | VYYCVRDGSGSYWGQGTLVTVSSASTKGPSVFPLAPSSKSTSGGTA |
| | chain | | | ALGCLVKDYFPEPVTVSWNSGALTSGVHTFPAVLQSSGLYSLSSVV |
| | sequence | | | TVPSSSLGTQTYICNVNHKPSNTKVDKKVEPKSCDKTHTCPPCPAP |
| | | | | ELLGGPSVFLFPPKPKDTLMISRTPEVTCVVVDVSHEDPEVKFNWY |
| | | | | VDGVEVHNAKTKPREEQYNSTYRVVSVLTVLHQDWLNGKEYKCKVS |
| | | | | NKALPAPIEKTISKAKGQPREPQVYTLPPSRDELTKNQVSLTCLVK |
| | | | | GFYPSDIAVEWESNGQPENNYKTTPPVLDSDGSFFLYSKLTVDKSR |
| | | | | WQQGNVFSCSVMHEALHNHYTQKSLSLSPGK |
| 161 | 386H03 - | Nucleic acid | | CAGGTGCAGCTGCAGGAGTCGGGCCCAGGACTGGTGAAGCCTTCGG |
| | full | sequence of 386H03 | | GGACCCTGTCCCTCACCTGCGCTGTCTCTGGTGGCTCCATCAGCAG |
| | heavy | heavy chain | | TAGTGACTGGTGGAGTTGGGTCCGCCAGCCCCCAGGGAAGGGGCTG |
| | chain | | | GAGTGGATTGGGGAAATCTTTCATAGTGGGAACACCAACTACAACC |
| | sequence | | | CGTCCCTCAAGAGTCGAGTCACCATATCAGTAGACAAGTCCAAGAA |
| | | | | CCAGATCTCCCTGAGGCTGAACTCTGTGACCGCCGCGGACACGGCC |
| | | | | GTGTATTACTGTGTGAGAGATGGTTCGGGGAGTTACTGGGGCCAGG |
| | | | | GAACCCTGGTCACCGTCTCCTCAGCCAGCACCAAGGGCCCCTCTGT |
| | | | | GTTCCCTCTGGCCCCTTCCAGCAAGTCCACCTCTGGCGGAACAGCC |
| | | | | GCTCTGGGCTGCCTCGTGAAGGACTACTTCCCCGAGCCTGTGACCG |
| | | | | TGTCCTGGAACTCTGGCGCTCTGACCAGCGGAGTGCACACCTTCCC |
| | | | | TGCTGTGCTGCAGTCCTCCGGCCTGTACTCCCTGTCCTCCGTCGTG |
| | | | | ACCGTGCCTTCCAGCTCTCTGGGCACCCAGACCTACATCTGCAACG |
| | | | | TGAACCACAAGCCCTCCAACACCAAGGTGGACAAGAAGGTGGAACC |
| | | | | CAAGTCCTGCGACAAGACCCACACCTGTCCCCCTTGTCCTGCCCCT |
| | | | | GAACTGCTGGGCGGACCTTCCGTGTTCCTGTTCCCCCCAAAGCCCA |
| | | | | AGGACACCCTGATGATCTCCCGGACCCCCGAAGTGACCTGCGTGGT |
| | | | | GGTGGATGTGTCCCACGAGGACCCTGAAGTGAAGTTCAATTGGTAC |
| | | | | GTGGACGGCGTGGAAGTGCACAACGCCAAGACCAAGCCTAGAGAGG |
| | | | | AACAGTACAACTCCACCTACCGGGTGGTGTCCGTGCTGACCGTGCT |
| | | | | GCACCAGGATTGGCTGAACGGCAAAGAGTACAAGTGCAAGGTGTCC |
| | | | | AACAAGGCCCTGCCTGCCCCCATCGAAAAGACCATCTCCAAGGCCA |
| | | | | AGGGCCAGCCCCGGGAACCCCAGGTGTACACACTGCCCCCTAGCAG |
| | | | | GGACGAGCTGACCAAGAACCAGGTGTCCCTGACCTGTCTCGTGAAA |
| | | | | GGCTTCTACCCCTCCGATATCGCCGTGGAATGGGAGTCCAACGGCC |
| | | | | AGCCTGAGAACAACTACAAGACCACCCCCCCTGTGCTGGACTCCGA |
| | | | | CGGCTCATTCTTCCTGTACAGCAAGCTGACAGTGGACAAGTCCCGG |
| | | | | TGGCAGCAGGGCAACGTGTTCTCCTGCTCCGTGATGCACGAGGCCC |
| | | | | TGCACAACCACTACACCCAGAAGTCCCTGTCCCTGAGCCCCGGCAA |
| | | | | G |
| 162 | 386H03 - | Amino acid | | QSVLYSSNNKNY |
| | CDRL1 | sequence of CDRL1 | | |
| | (IMGT) | of 386H03 using | | |
| | | IMGT | | |
| 163 | 386H03 - | Amino acid | | WAS |
| | CDRL2 | sequence of CDRL2 | | |
| | (IMGT) | of 386H03 using | | |
| | | IMGT | | |
| 164 | 386H03 - | Amino acid | | QQYYSTRS |
| | CDRL3 | sequence of CDRL3 | | |
| | (IMGT) | of 386H03 using | | |
| | | IMGT | | |
| 165 | 386H03 - | Amino acid | | KSSQSVLYSSNNKNYLA |
| | CDRL1 | sequence of CDRL1 | | |
| | (Kabat) | of 386H03 using | | |
| | | Kabat | | |
| 166 | 386H03 - | Amino acid | | WASTRES |
| | CDRL2 | sequence of CDRL2 | | |
| | (Kabat) | of 386H03 using | | |
| | | Kabat | | |
| 167 | 386H03 - | Amino acid | | QQYYSTRS |
| | CDRL3 | sequence of CDRL3 | | |
| | (Kabat) | of 386H03 using | | |
| | | Kabat | | |
| 168 | 386H03 - | Amino acid | | DIVMTQSPDSLAVSLGERATINCKSSQSVLYSSNNKNYLAWYQQKP |
| | Light | sequence of V_{L} of | | GQPPKLLIYWASTRESGVPDRFSGSGSGTDFTLTISSLQAEDVAVY |
| | chain | 386H03 | | YCQQYYSTRSFGQGTKLEIK |
| | variable | | | |
| | region | | | |
| 169 | 386H03 - | Nucleic acid | | GACATCGTGATGACCCAGTCTCCAGACTCCCTGGCTGTGTCTCTGG |
| | Light | sequence of V_{L} of | | GCGAGAGGGCCACCATCAACTGCAAGTCCAGCCAGAGTGTTTTATA |
| | chain | 386H03 | | CAGCTCCAACAATAAGAACTACTTAGCTTGGTACCAGCAGAAACCA |
| | variable | | | GGACAGCCTCCTAAACTGCTCATTTACTGGGCATCTACCCGGGAAT |
| | region | | | CCGGGGTCCCTGACCGATTCAGTGGCAGCGGGTCTGGGACAGATTT |
| | | | | CACTCTCACCATCAGCAGCCTGCAGGCTGAAGATGTGGCAGTTTAT |
| | | | | TACTGTCAGCAATATTATAGTACTCGCAGTTTTGGCCAGGGGACCA |
| | | | | AGCTGGAGATCAAAC |
| 170 | 386H03 - | Amino acid | | DIVMTQSPDSLAVSLGERATINCKSSQSVLYSSNNKNYLAWYQQKP |
| | full | sequence of 386H03 | | GQPPKLLIYWASTRESGVPDRFSGSGSGTDFTLTISSLQAEDVAVY |
| | light | light chain | | YCQQYYSTRSFGQGTKLEIKRTVAAPSVFIFPPSDEQLKSGTASVV |
| | chain | | | CLLNNFYPREAKVQWKVDNALQSGNSQESVTEQDSKDSTYSLSSTL |
| | sequence | | | TLSKADYEKHKVYACEVTHQGLSSPVTKSFNRGEC |
| 171 | 386H03 - | Nucleic acid | | GACATCGTGATGACCCAGTCTCCAGACTCCCTGGCTGTGTCTCTGG |
| | full | sequence of 386H03 | | GCGAGAGGGCCACCATCAACTGCAAGTCCAGCCAGAGTGTTTTATA |
| | light | light chain | | CAGCTCCAACAATAAGAACTACTTAGCTTGGTACCAGCAGAAACCA |
| | chain | | | GGACAGCCTCCTAAACTGCTCATTTACTGGGCATCTACCCGGGAAT |
| | sequence | | | CCGGGGTCCCTGACCGATTCAGTGGCAGCGGGTCTGGGACAGATTT |
| | | | | CACTCTCACCATCAGCAGCCTGCAGGCTGAAGATGTGGCAGTTTAT |
| | | | | TACTGTCAGCAATATTATAGTACTCGCAGTTTTGGCCAGGGGACCA |
| | | | | AGCTGGAGATCAAACGTACGGTGGCCGCTCCCTCCGTGTTCATCTT |
| | | | | CCCACCTTCCGACGAGCAGCTGAAGTCCGGCACCGCTTCTGTCGTG |
| | | | | TGCCTGCTGAACAACTTCTACCCCCGCGAGGCCAAGGTGCAGTGGA |
| | | | | AGGTGGACAACGCCCTGCAGTCCGGCAACTCCCAGGAATCCGTGAC |
| | | | | CGAGCAGGACTCCAAGGACAGCACCTACTCCCTGTCCTCCACCCTG |
| | | | | ACCCTGTCCAAGGCCGACTACGAGAAGCACAAGGTGTACGCCTGCG |
| | | | | AAGTGACCCACCAGGGCCTGTCTAGCCCCGTGACCAAGTCTTTCAA |
| | | | | CCGGGGCGAGTGT |
| 172 | 389A03 - | Amino acid | | GGSISSSSYY |
| | CDRH1 | sequence of CDRH1 | | |
| | (IMGT) | of 389A03 using | | |
| | | IMGT | | |
| 173 | 389A03 - | Amino acid | | IYSTGYT |
| | CDRH2 | sequence of CDRH2 | | |
| | (IMGT) | of 389A03 using | | |
| | | IMGT | | |
| 174 | 389A03 - | Amino acid | | AISTAAGPEYFHR |
| | CDRH3 | sequence of CDRH3 | | |
| | (IMGT) | of 389A03 using | | |
| | | IMGT | | |
| 175 | 389A03 - | Amino acid | | SSSYYCG |
| | CDRH1 | sequence of CDRH1 | | |
| | (Kabat) | of 389A03 using | | |
| | | Kabat | | |
| 176 | 389A03 - | Amino acid | | SIYSTGYTYYNPSLKS |
| | CDRH2 | sequence of CDRH2 | | |
| | (Kabat) | of 389A03 using | | |
| | | Kabat | | |
| 177 | 389A03 - | Amino acid | | STAAGPEYFHR |
| | CDRH3 | sequence of CDRH3 | | |
| | (Kabat) | of 389A03 using | | |
| | | Kabat | | |
| 178 | 389A03 - | Amino acid | | QLQESGPGLVKPSETLSLTCTVSGGSISSSSYYCGWIRQPPGKGLD |
| | Heavy | sequence of V_{H} of | | WIGSIYSTGYTYYNPSLKSRVTISIDTSKNQFSCLILTSVTAADTA |
| | chain | 389A03 | | VYYCAISTAAGPEYFHRWGQGTLVTVSS |
| | variable | | | |
| | region | | | |
| 179 | 389A03 - | Nucleic acid | | CAGCTGCAGGAGTCGGGCCCAGGCCTGGTGAAGCCTTCGGAGACCC |
| | Heavy | sequence of V_{H} of | | TGTCCCTCACCTGCACTGTCTCTGGTGGCTCCATCAGCAGTAGTAG |
| | chain | 389A03 | | TTATTACTGCGGCTGGATCCGCCAGCCCCCTGGGAAGGGGCTGGAC |
| | variable | | | TGGATTGGGAGTATCTATTCTACTGGGTACACCTACTACAACCCGT |
| | region | | | CCCTCAAGAGTCGAGTCACCATTTCCATAGACACGTCCAAGAACCA |
| | | | | GTTCTCATGCCTGATACTGACCTCTGTGACCGCCGCAGACACGGCT |
| | | | | GTGTATTACTGTGCGATAAGTACAGCAGCTGGCCCTGAATACTTCC |
| | | | | ATCGCTGGGGCCAGGGCACCCTGGTCACCGTCTCCTCAG |
| 180 | 389A03 - | Amino acid | | QLQESGPGLVKPSETLSLTCTVSGGSISSSSYYCGWIRQPPGKGLD |
| | full | sequence of 389A03 | | WIGSIYSTGYTYYNPSLKSRVTISIDTSKNQFSCLILTSVTAADTA |
| | heavy | heavy chain | | VYYCAISTAAGPEYFHRWGQGTLVTVSSASTKGPSVFPLAPSSKST |
| | chain | | | SGGTAALGCLVKDYFPEPVTVSWNSGALTSGVHTFPAVLQSSGLYS |
| | sequence | | | LSSVVTVPSSSLGTQTYICNVNHKPSNTKVDKKVEPKSCDKTHTCP |
| | | | | PCPAPELLGGPSVFLFPPKPKDTLMISRTPEVTCVVVDVSHEDPEV |
| | | | | KFNWYVDGVEVHNAKTKPREEQYNSTYRVVSVLTVLHQDWLNGKEY |
| | | | | KCKVSNKALPAPIEKTISKAKGQPREPQVYTLPPSRDELTKNQVSL |
| | | | | TCLVKGFYPSDIAVEWESNGQPENNYKTTPPVLDSDGSFFLYSKLT |
| | | | | VDKSRWQQGNVFSCSVMHEALHNHYTQKSLSLSPGK |
| 181 | 389A03 - | Nucleic acid | | CAGCTGCAGGAGTCGGGCCCAGGCCTGGTGAAGCCTTCGGAGACCC |
| | full | sequence of 389A03 | | TGTCCCTCACCTGCACTGTCTCTGGTGGCTCCATCAGCAGTAGTAG |
| | heavy | heavy chain | | TTATTACTGCGGCTGGATCCGCCAGCCCCCTGGGAAGGGGCTGGAC |
| | chain | | | TGGATTGGGAGTATCTATTCTACTGGGTACACCTACTACAACCCGT |
| | sequence | | | CCCTCAAGAGTCGAGTCACCATTTCCATAGACACGTCCAAGAACCA |
| | | | | GTTCTCATGCCTGATACTGACCTCTGTGACCGCCGCAGACACGGCT |
| | | | | GTGTATTACTGTGCGATAAGTACAGCAGCTGGCCCTGAATACTTCC |
| | | | | ATCGCTGGGGCCAGGGCACCCTGGTCACCGTCTCCTCAGCCAGCAC |
| | | | | CAAGGGCCCCTCTGTGTTCCCTCTGGCCCCTTCCAGCAAGTCCACC |
| | | | | TCTGGCGGAACAGCCGCTCTGGGCTGCCTCGTGAAGGACTACTTCC |
| | | | | CCGAGCCTGTGACCGTGTCCTGGAACTCTGGCGCTCTGACCAGCGG |
| | | | | AGTGCACACCTTCCCTGCTGTGCTGCAGTCCTCCGGCCTGTACTCC |
| | | | | CTGTCCTCCGTCGTGACCGTGCCTTCCAGCTCTCTGGGCACCCAGA |
| | | | | CCTACATCTGCAACGTGAACCACAAGCCCTCCAACACCAAGGTGGA |
| | | | | CAAGAAGGTGGAACCCAAGTCCTGCGACAAGACCCACACCTGTCCC |
| | | | | CCTTGTCCTGCCCCTGAACTGCTGGGCGGACCTTCCGTGTTCCTGT |
| | | | | TCCCCCCAAAGCCCAAGGACACCCTGATGATCTCCCGGACCCCCGA |
| | | | | AGTGACCTGCGTGGTGGTGGATGTGTCCCACGAGGACCCTGAAGTG |
| | | | | AAGTTCAATTGGTACGTGGACGGCGTGGAAGTGCACAACGCCAAGA |
| | | | | CCAAGCCTAGAGAGGAACAGTACAACTCCACCTACCGGGTGGTGTC |
| | | | | CGTGCTGACCGTGCTGCACCAGGATTGGCTGAACGGCAAAGAGTAC |
| | | | | AAGTGCAAGGTGTCCAACAAGGCCCTGCCTGCCCCCATCGAAAAGA |
| | | | | CCATCTCCAAGGCCAAGGGCCAGCCCCGGGAACCCCAGGTGTACAC |
| | | | | ACTGCCCCCTAGCAGGGACGAGCTGACCAAGAACCAGGTGTCCCTG |
| | | | | ACCTGTCTCGTGAAAGGCTTCTACCCCTCCGATATCGCCGTGGAAT |
| | | | | GGGAGTCCAACGGCCAGCCTGAGAACAACTACAAGACCACCCCCCC |
| | | | | TGTGCTGGACTCCGACGGCTCATTCTTCCTGTACAGCAAGCTGACA |
| | | | | GTGGACAAGTCCCGGTGGCAGCAGGGCAACGTGTTCTCCTGCTCCG |
| | | | | TGATGCACGAGGCCCTGCACAACCACTACACCCAGAAGTCCCTGTC |
| | | | | CCTGAGCCCCGGCAAG |
| 182 | 389A03 - | Amino acid | | QSVLYSSNSKNF |
| | CDRL1 | sequence of CDRL1 | | |
| | (IMGT) | of 389A03 using | | |
| | | IMGT | | |
| 183 | 389A03 - | Amino acid | | WAS |
| | CDRL2 | sequence of CDRL2 | | |
| | (IMGT) | of 389A03 using | | |
| | | IMGT | | |
| 184 | 389A03 - | Amino acid | | QQYYSTPRT |
| | CDRL3 | sequence of CDRL3 | | |
| | (IMGT) | of 389A03 using | | |
| | | IMGT | | |
| 185 | 389A03 - | Amino acid | | KSSQSVLYSSNSKNFLA |
| | CDRL1 | sequence of CDRL1 | | |
| | (Kabat) | of 389A03 using | | |
| | | Kabat | | |
| 186 | 389A03 - | Amino acid | | WASTRGS |
| | CDRL2 | sequence of CDRL2 | | |
| | (Kabat) | of 389A03 using | | |
| | | Kabat | | |
| 187 | 389A03 - | Amino acid | | QQYYSTPRT |
| | CDRL3 | sequence of CDRL3 | | |
| | (Kabat) | of 389A03 using | | |
| | | Kabat | | |
| 188 | 389A03 - | Amino acid | | DIVMTQSPDSLAVSLGERATINCKSSQSVLYSSNSKNFLAWYQQKP |
| | Light | sequence of V_{L} of | | GQPPKLFIYWASTRGSGVPDRISGSGSGTDFNLTISSLQAEDVAVY |
| | chain | 389A03 | | YCQQYYSTPRTFGQGTKVEIK |
| | variable | | | |
| | region | | | |
| 189 | 389A03 - | Nucleic acid | | GACATCGTGATGACCCAGTCTCCAGACTCCCTGGCTGTGTCTCTGG |
| | Light | sequence of V_{L} of | | GCGAGAGGGCCACCATCAACTGCAAGTCCAGCCAGAGTGTTTTATA |
| | chain | 389A03 | | CAGCTCCAACAGTAAGAACTTCTTAGCTTGGTACCAGCAGAAACCG |
| | variable | | | GGACAGCCTCCTAAGCTGTTCATTTACTGGGCATCTACCCGGGGAT |
| | region | | | CCGGGGTCCCTGACCGAATCAGTGGCAGCGGGTCTGGGACAGATTT |
| | | | | CAATCTCACCATCAGCAGCCTGCAGGCTGAAGATGTGGCAGTTTAT |
| | | | | TACTGTCAACAATATTATAGTACTCCTCGGACGTTCGGCCAAGGGA |
| | | | | CCAAGGTGGAGATCAAAC |
| 190 | 389A03 - | Amino acid | | DIVMTQSPDSLAVSLGERATINCKSSQSVLYSSNSKNFLAWYQQKP |
| | full | sequence of 389A03 | | GQPPKLFIYWASTRGSGVPDRISGSGSGTDFNLTISSLQAEDVAVY |
| | light | light chain | | YCQQYYSTPRTFGQGTKVEIKRTVAAPSVFIFPPSDEQLKSGTASV |
| | chain | | | VCLLNNFYPREAKVQWKVDNALQSGNSQESVTEQDSKDSTYSLSST |
| | sequence | | | LTLSKADYEKHKVYACEVTHQGLSSPVTKSFNRGEC |
| 191 | 389A03 - | Nucleic acid | | GACATCGTGATGACCCAGTCTCCAGACTCCCTGGCTGTGTCTCTGG |
| | full | sequence of 389A03 | | GCGAGAGGGCCACCATCAACTGCAAGTCCAGCCAGAGTGTTTTATA |
| | light | light chain | | CAGCTCCAACAGTAAGAACTTCTTAGCTTGGTACCAGCAGAAACCG |
| | chain | | | GGACAGCCTCCTAAGCTGTTCATTTACTGGGCATCTACCCGGGGAT |
| | sequence | | | CCGGGGTCCCTGACCGAATCAGTGGCAGCGGGTCTGGGACAGATTT |
| | | | | CAATCTCACCATCAGCAGCCTGCAGGCTGAAGATGTGGCAGTTTAT |
| | | | | TACTGTCAACAATATTATAGTACTCCTCGGACGTTCGGCCAAGGGA |
| | | | | CCAAGGTGGAGATCAAACGTACGGTGGCCGCTCCCTCCGTGTTCAT |
| | | | | CTTCCCACCTTCCGACGAGCAGCTGAAGTCCGGCACCGCTTCTGTC |
| | | | | GTGTGCCTGCTGAACAACTTCTACCCCCGCGAGGCCAAGGTGCAGT |
| | | | | GGAAGGTGGACAACGCCCTGCAGTCCGGCAACTCCCAGGAATCCGT |
| | | | | GACCGAGCAGGACTCCAAGGACAGCACCTACTCCCTGTCCTCCACC |
| | | | | CTGACCCTGTCCAAGGCCGACTACGAGAAGCACAAGGTGTACGCCT |
| | | | | GCGAAGTGACCCACCAGGGCCTGTCTAGCCCCGTGACCAAGTCTTT |
| | | | | CAACCGGGGCGAGTGT |
| 192 | Human | IGHG* | Heavy Chain | gcttccaccaagggcccatccgtcttccccctggcgccctgctcca |
| | IgG4 | 01 & | Constant | ggagcacctccgagagcacagccgccctgggctgcctggtcaagga |
| | heavy | IGHG4 | Region | ctacttccccgaaccggtgacggtgtcgtggaactcaggcgccctg |
| | chain | *04 | Nucleotide | accagcggcgtgcacaccttcccggctgtcctacagtcctcaggac |
| | constant | | Sequence | tctactccctcagcagcgtggtgaccgtgccctccagcagcttggg |
| | region | | | cacgaagacctacacctgcaacgtagatcacaagcccagcaacacc |
| | #1 | | | aaggtggacaagagagttgagtccaaatatggtcccccatgcccat |
| | | | | catgcccagcacctgagttcctggggggaccatcagtcttcctgtt |
| | | | | ccccccaaaacccaaggacactctcatgatctcccggacccctgag |
| | | | | gtcacgtgcgtggtggtggacgtgagccaggaagaccccgaggtcc |
| | | | | agttcaactggtacgtggatggcgtggaggtgcataatgccaagac |
| | | | | aaagccgcgggaggagcagttcaacagcacgtaccgtgtggtcagc |
| | | | | gtcctcaccgtcctgcaccaggactggctgaacggcaaggagtaca |
| | | | | agtgcaaggtctccaacaaaggcctcccgtcctccatcgagaaaac |
| | | | | catctccaaagccaaagggcagccccgagagccacaggtgtacacc |
| | | | | ctgcccccatcccaggaggagatgaccaagaaccaggtcagcctga |
| | | | | cctgcctggtcaaaggcttctaccccagcgacatcgccgtggagtg |
| | | | | ggagagcaatgggcagccggagaacaactacaagaccacgcctccc |
| | | | | gtgctggactccgacggctccttcttcctctacagcaggctaaccg |
| | | | | tggacaagagcaggtggcaggaggggaatgtcttctcatgctccgt |
| | | | | gatgcatgaggctctgcacaaccactacacacagaagagcctctcc |
| | | | | ctgtctctgggtaaa |
| 193 | | | Heavy Chain | ASTKGPSVFPLAPCSRSTSESTAALGCLVKDYFPEPVTVSWNSGAL |
| | | | Constant | TSGVHTFPAVLQSSGLYSLSSVVTVPSSSLGTKTYTCNVDHKPSNT |
| | | | Region | KVDKRVESKYGPPCPSCPAPEFLGGPSVFLFPPKPKDTLMISRTPE |
| | | | Amino Acid | VTCVVVDVSQEDPEVQFNWYVDGVEVHNAKTKPREEQFNSTYRVVS |
| | | | Sequence | VLTVLHQDWLNGKEYKCKVSNKGLPSSIEKTISKAKGQPREPQVYT |
| | | | | LPPSQEEMTKNQVSLTCLVKGFYPSDIAVEWESNGQPENNYKTTPP |
| | | | | VLDSDGSFFLYSRLTVDKSRWQEGNVFSCSVMHEALHNHYTQKSLS |
| | | | | LSLGK |
| 194 | Human | IGHG* | Heavy Chain | gcttccaccaagggcccatccgtcttccccctggcgccctgctcca |
| | IgG4 | 02 | Constant | ggagcacctccgagagcacagccgccctgggctgcctggtcaagga |
| | heavy | | Region | ctacttccccgaaccggtgacggtgtcgtggaactcaggcgccctg |
| | chain | | Nucleotide | accagcggcgtgcacaccttcccggctgtcctacagtcctcaggac |
| | constant | | Sequence | tctactccctcagcagcgtggtgaccgtgccctccagcagcttggg |
| | region | | | cacgaagacctacacctgcaacgtagatcacaagcccagcaacacc |
| | #2 | | | aaggtggacaagagagttgagtccaaatatggtcccccgtgcccat |
| | | | | catgcccagcacctgagttcctggggggaccatcagtcttcctgtt |
| | | | | ccccccaaaacccaaggacactctcatgatctcccggacccctgag |
| | | | | gtcacgtgcgtggtggtggacgtgagccaggaagaccccgaggtcc |
| | | | | agttcaactggtacgtggatggcgtggaggtgcataatgccaagac |
| | | | | aaagccgcgggaggagcagttcaacagcacgtaccgtgtggtcagc |
| | | | | gtcctcaccgtcgtgcaccaggactggctgaacggcaaggagtaca |
| | | | | agtgcaaggtctccaacaaaggcctcccgtcctccatcgagaaaac |
| | | | | catctccaaagccaaagggcagccccgagagccacaggtgtacacc |
| | | | | ctgcccccatcccaggaggagatgaccaagaaccaggtcagcctga |
| | | | | cctgcctggtcaaaggcttctaccccagcgacatcgccgtggagtg |
| | | | | ggagagcaatgggcagccggagaacaactacaagaccacgcctccc |
| | | | | gtgctggactccgacggctccttcttcctctacagcaggctaaccg |
| | | | | tggacaagagcaggtggcaggaggggaatgtcttctcatgctccgt |
| | | | | gatgcatgaggctctgcacaaccactacacgcagaagagcctctcc |
| | | | | ctgtctctgggtaaa |
| 195 | | | Heavy Chain | ASTKGPSVFPLAPCSRSTSESTAALGCLVKDYFPEPVTVSWNSGAL |
| | | | Constant | TSGVHTFPAVLQSSGLYSLSSVVTVPSSSLGTKTYTCNVDHKPSNT |
| | | | Region | KVDKRVESKYGPPCPSCPAPEFLGGPSVFLFPPKPKDTLMISRTPE |
| | | | Amino Acid | VTCVVVDVSQEDPEVQFNWYVDGVEVHNAKTKPREEQFNSTYRVVS |
| | | | Sequence | VLTVVHQDWLNGKEYKCKVSNKGLPSSIEKTISKAKGQPREPQVYT |
| | | | | LPPSQEEMTKNQVSLTCLVKGFYPSDIAVEWESNGQPENNYKTTPP |
| | | | | VLDSDGSFFLYSRLTVDKSRWQEGNVFSCSVMHEALHNHYTQKSLS |
| | | | | LSLGK |
| 196 | Human | IGHG* | Heavy Chain | gcttccaccaagggcccatccgtcttccccctggcgccctgctcca |
| | IgG4 | 03 | Constant | ggagcacctccgagagcacagccgccctgggctgcctggtcaagga |
| | heavy | | Region | ctacttccccgaaccggtgacggtgtcgtggaactcaggcgccctg |
| | chain | | Nucleotide | accagcggcgtgcacaccttcccggctgtcctacagtcctcaggac |
| | constant | | Sequence | tctactccctcagcagcgtggtgaccgtgccctccagcagcttggg |
| | region | | | cacgaagacctacacctgcaacgtagatcacaagcccagcaacacc |
| | #3 | | | aaggtggacaagagagttgagtccaaatatggtcccccatgcccat |
| | | | | catgcccagcacctgagttcctggggggaccatcagtcttcctgtt |
| | | | | ccccccaaaacccaaggacactctcatgatctcccggacccctgag |
| | | | | gtcacgtgcgtggtggtggacgtgagccaggaagaccccgaggtcc |
| | | | | agttcaactggtacgtggatggcgtggaggtgcataatgccaagac |
| | | | | aaagccgcgggaggagcagttcaacagcacgtaccgtgtggtcagc |
| | | | | gtcctcaccgtcctgcaccaggactggctgaacggcaaggagtaca |
| | | | | agtgcaaggtctccaacaaaggcctcccgtcctccatcgagaaaac |
| | | | | catctccaaagccaaagggcagccccgagagccacaggtgtacacc |
| | | | | ctgcccccatcccaggaggagatgaccaagaaccaggtcagcctga |
| | | | | cctgcctggtcaaaggcttctaccccagcgacatcgccgtggagtg |
| | | | | ggagagcaatgggcagccggagaacaactacaagaccacgcctccc |
| | | | | gtgctggactccgacggctccttcttcctctacagcaagctcaccg |
| | | | | tggacaagagcaggtggcaggaggggaacgtcttctcatgctccgt |
| | | | | gatgcatgaggctctgcacaaccactacacgcagaagagcctctcc |
| | | | | ctgtctctgggtaaa |
| 197 | | | Heavy Chain | ASTKGPSVFPLAPCSRSTSESTAALGCLVKDYFPEPVTVSWNSGAL |
| | | | Constant | TSGVHTFPAVLQSSGLYSLSSVVTVPSSSLGTKTYTCNVDHKPSNT |
| | | | Region | KVDKRVESKYGPPCPSCPAPEFLGGPSVFLFPPKPKDTLMISRTPE |
| | | | Amino Acid | VTCVVVDVSQEDPEVQFNWYVDGVEVHNAKTKPREEQFNSTYRVVS |
| | | | Sequence | VLTVLHQDWLNGKEYKCKVSNKGLPSSIEKTISKAKGQPREPQVYT |
| | | | | LPPSQEEMTKNQVSLTCLVKGFYPSDIAVEWESNGQPENNYKTTPP |
| | | | | VLDSDGSFFLYSKLTVDKSRWQEGNVFSCSVMHEALHNHYTQKSLS |
| | | | | LSLGK |
| 198 | IgG4 | - | Heavy Chain | gcctccaccaagggcccatccgtcttccccctggcgccctgctcca |
| | heavy | IgG4- | Constant | ggagcacctccgagagcacggccgccctgggctgcctggtcaagga |
| | chain | PE | Region | ctacttccccgaaccagtgacggtgtcgtggaactcaggcgccctg |
| | constant | | Nucleotide | accagcggcgtgcacaccttcccggctgtcctacagtcctcaggac |
| | region - | | Sequence - | tctactccctcagcagcgtggtgaccgtgccctccagcagcttggg |
| | IgG4-PE | | Synthetic | cacgaagacctacacctgcaacgtagatcacaagcccagcaacacc |
| | | | Version A | aaggtggacaagagagttgagtccaaatatggtcccccatgcccac |
| | | | | catgcccagcgcctgaatttgaggggggaccatcagtcttcctgtt |
| | | | | ccccccaaaacccaaggacactctcatgatctcccggacccctgag |
| | | | | gtcacgtgcgtggtggtggacgtgagccaggaagaccccgaggtcc |
| | | | | agttcaactggtacgtggatggcgtggaggtgcataatgccaagac |
| | | | | aaagccgcgggaggagcagttcaacagcacgtaccgtgtggtcagc |
| | | | | gtcctcaccgtcctgcaccaggactggctgaacggcaaggagtaca |
| | | | | agtgcaaggtctccaacaaaggcctcccgtcatcgatcgagaaaac |
| | | | | catctccaaagccaaagggcagccccgagagccacaggtgtacacc |
| | | | | ctgcccccatcccaggaggagatgaccaagaaccaggtcagcctga |
| | | | | cctgcctggtcaaaggcttctaccccagcgacatcgccgtggagtg |
| | | | | ggagagcaatgggcagccggagaacaactacaagaccacgcctccc |
| | | | | gtgctggactccgacggatccttcttcctctacagcaggctaaccg |
| | | | | tggacaagagcaggtggcaggaggggaatgtcttctcatgctccgt |
| | | | | gatgcatgaggctctgcacaaccactacacacagaagagcctctcc |
| | | | | ctgtctctgggtaaa |
| 199 | IgG4 | | Heavy Chain | ASTKGPSVFPLAPCSRSTSESTAALGCLVKDYFPEPVTVSWNSGAL |
| | heavy | | Constant | TSGVHTFPAVLQSSGLYSLSSVVTVPSSSLGTKTYTCNVDHKPSNT |
| | chain | | Region | KVDKRVESKYGPPCPPCPAPEFEGGPSVFLFPPKPKDTLMISRTPE |
| | constant | | Amino Acid | VTCVVVDVSQEDPEVQFNWYVDGVEVHNAKTKPREEQFNSTYRVVS |
| | region - | | Sequence - | VLTVLHQDWLNGKEYKCKVSNKGLPSSIEKTISKAKGQPREPQVYT |
| | IgG4-PE | | Encoded by | LPPSQEEMTKNQVSLTCLVKGFYPSDIAVEWESNGQPENNYKTTPP |
| | | | Synthetic | VLDSDGSFFLYSRLTVDKSRWQEGNVFSCSVMHEALHNHYTQKSLS |
| | | | Version A, | LSLGK |
| | | | B & C(Two | |
| | | | residues | |
| | | | that differ | |
| | | | from the | |
| | | | wild-type | |
| | | | sequence | |
| | | | are | |
| | | | identified | |
| | | | in bold) | |
| 200 | IgG4 | | Heavy Chain | Gcctccaccaagggacctagcgtgttccctctcgccccctgttcca |
| | heavy | | Constant | ggtccacaagcgagtccaccgctgccctcggctgtctggtgaaaga |
| | chain | | Region | ctactttcccgagcccgtgaccgtctcctggaatagcggagccctg |
| | constant | | Nucleotide | acctccggcgtgcacacatttcccgccgtgctgcagagcagcggac |
| | region- | | Sequence - | tgtatagcctgagcagcgtggtgaccgtgcccagctccagcctcgg |
| | IgG4-PE | | Synthetic | caccaaaacctacacctgcaacgtggaccacaagccctccaacacc |
| | | | Version B | aaggtggacaagcgggtggagagcaagtacggccccccttgccctc |
| | | | | cttgtcctgcccctgagttcgagggaggaccctccgtgttcctgtt |
| | | | | tccccccaaacccaaggacaccctgatgatctcccggacacccgag |
| | | | | gtgacctgtgtggtcgtggacgtcagccaggaggaccccgaggtgc |
| | | | | agttcaactggtatgtggacggcgtggaggtgcacaatgccaaaac |
| | | | | caagcccagggaggagcagttcaattccacctacagggtggtgagc |
| | | | | gtgctgaccgtcctgcatcaggattggctgaacggcaaggagtaca |
| | | | | agtgcaaggtgtccaacaagggactgcccagctccatcgagaagac |
| | | | | catcagcaaggctaagggccagccgagggagccccaggtgtatacc |
| | | | | ctgcctcctagccaggaagagatgaccaagaaccaagtgtccctga |
| | | | | cctgcctggtgaagggattctacccctccgacatcgccgtggagtg |
| | | | | ggagagcaatggccagcccgagaacaactacaaaacaacccctccc |
| | | | | gtgctcgatagcgacggcagcttctttctctacagccggctgacag |
| | | | | tggacaagagcaggtggcaggagggcaacgtgttctcctgttccgt |
| | | | | gatgcacgaggccctgcacaatcactacacccagaagagcctctcc |
| | | | | ctgtccctgggcaag |
| 201 | IgG4 | | Heavy Chain | gccagcaccaagggcccttccgtgttccccctggccccttgcagca |
| | heavy | | Constant | ggagcacctccgaatccacagctgccctgggctgtctggtgaagga |
| | chain | | Region | ctactttcccgagcccgtgaccgtgagctggaacagcggcgctctg |
| | constant | | Nucleotide | acatccggcgtccacacctttcctgccgtcctgcagtcctccggcc |
| | region- | | Sequence - | tctactccctgtcctccgtggtgaccgtgcctagctcctccctcgg |
| | IgG4-PE | | Synthetic | caccaagacctacacctgtaacgtggaccacaaaccctccaacacc |
| | | | Version C | aaggtggacaaacgggtcgagagcaagtacggccctccctgccctc |
| | | | | cttgtcctgcccccgagttcgaaggcggacccagcgtgttcctgtt |
| | | | | ccctcctaagcccaaggacaccctcatgatcagccggacacccgag |
| | | | | gtgacctgcgtggtggtggatgtgagccaggaggaccctgaggtcc |
| | | | | agttcaactggtatgtggatggcgtggaggtgcacaacgccaagac |
| | | | | aaagccccgggaagagcagttcaactccacctacagggtggtcagc |
| | | | | gtgctgaccgtgctgcatcaggactggctgaacggcaaggagtaca |
| | | | | agtgcaaggtcagcaataagggactgcccagcagcatcgagaagac |
| | | | | catctccaaggctaaaggccagccccgggaacctcaggtgtacacc |
| | | | | ctgcctcccagccaggaggagatgaccaagaaccaggtgagcctga |
| | | | | cctgcctggtgaagggattctacccttccgacatcgccgtggagtg |
| | | | | ggagtccaacggccagcccgagaacaattataagaccacccctccc |
| | | | | gtcctcgacagcgacggatccttctttctgtactccaggctgaccg |
| | | | | tggataagtccaggtggcaggaaggcaacgtgttcagctgctccgt |
| | | | | gatgcacgaggccctgcacaatcactacacccagaagtccctgagc |
| | | | | ctgtccctgggaaag |
| 202 | IgG4 | | Heavy Chain | gcctccaccaagggcccatccgtcttccccctggcgccctgctcca |
| | heavy | | Constant | ggagcacctccgagagcacggccgccctgggctgcctggtcaagga |
| | chain | | Region | ctacttccccgaaccagtgacggtgtcgtggaactcaggcgccctg |
| | constant | | Nucleotide | accagcggcgtgcacaccttcccggctgtcctacagtcctcaggac |
| | region | | Sequence - | tctactccctcagcagcgtggtgaccgtgccctccagcagcttggg |
| | | | Synthetic | cacgaagacctacacctgcaacgtagatcacaagcccagcaacacc |
| | | | Version D | aaggtggacaagagagttgagtccaaatatggtcccccatgcccac |
| | | | | catgcccagcgcctccagttgcggggggaccatcagtcttcctgtt |
| | | | | ccccccaaaacccaaggacactctcatgatctcccggacccctgag |
| | | | | gtcacgtgcgtggtggtggacgtgagccaggaagaccccgaggtcc |
| | | | | agttcaactggtacgtggatggcgtggaggtgcataatgccaagac |
| | | | | aaagccgcgggaggagcagttcaacagcacgtaccgtgtggtcagc |
| | | | | gtcctcaccgtcctgcaccaggactggctgaacggcaaggagtaca |
| | | | | agtgcaaggtctccaacaaaggcctcccgtcatcgatcgagaaaac |
| | | | | catctccaaagccaaagggcagccccgagagccacaggtgtacacc |
| | | | | ctgcccccatcccaggaggagatgaccaagaaccaggtcagcctga |
| | | | | cctgcctggtcaaaggcttctaccccagcgacatcgccgtggagtg |
| | | | | ggagagcaatgggcagccggagaacaactacaagaccacgcctccc |
| | | | | gtgctggactccgacggatccttcttcctctacagcaggctaaccg |
| | | | | tggacaagagcaggtggcaggaggggaatgtcttctcatgctccgt |
| | | | | gatgcatgaggctctgcacaaccactacacacagaagagcctctcc |
| | | | | ctgtctctgggtaaa |
| 203 | | | Heavy Chain | ASTKGPSVFPLAPCSRSTSESTAALGCLVKDYFPEPVTVSWNSGAL |
| | | | Constant | TSGVHTFPAVLQSSGLYSLSSVVTVPSSSLGTKTYTCNVDHKPSNT |
| | | | Region | KVDKRVESKYGPPCPPCPAPPVAGGPSVFLFPPKPKDTLMISRTPE |
| | | | Amino Acid | VTCVVVDVSQEDPEVQFNWYVDGVEVHNAKTKPREEQFNSTYRVVS |
| | | | Sequence - | VLTVLHQDWLNGKEYKCKVSNKGLPSSIEKTISKAKGQPREPQVYT |
| | | | encoded by | LPPSQEEMTKNQVSLTCLVKGFYPSDIAVEWESNGQPENNYKTTPP |
| | | | Synthetic | VLDSDGSFFLYSRLTVDKSRWQEGNVFSCSVMHEALHNHYTQKSLS |
| | | | Version D | LSLGK |
| 204 | Disabled | Disabled | Heavy Chain | gcctccaccaagggcccatcggtcttccccctggcaccctcctcca |
| | Human | IGHG1 | Constant | agagcacctctgggggcacagcggccctgggctgcctggtcaagga |
| | IgG1 | | Region | ctacttccccgaaccggtgacggtgtcgtggaactcaggcgccctg |
| | heavy | | Nucleotide | accagcggcgtgcacaccttcccggctgtcctacagtcctcaggac |
| | chain | | Sequence | tctactccctcagcagcgtggtgaccgtgccctccagcagcttggg |
| | constant | | | cacccagacctacatctgcaacgtgaatcacaagcccagcaacacc |
| | region | | | aaggtggacaagaaagtggagcccaaatcttgtgacaaaactcaca |
| | | | | catgcccaccgtgcccagcacctgaactcgcgggggcaccgtcagt |
| | | | | cttcctcttccccccaaaacccaaggacaccctcatgatctcccgg |
| | | | | acccctgaggtcacatgcgtggtggtggacgtgagccacgaagacc |
| | | | | ctgaggtcaagttcaactggtacgtggacggcgtggaggtgcataa |
| | | | | tgccaagacaaagccgcgggaggagcagtacaacagcacgtaccgt |
| | | | | gtggtcagcgtcctcaccgtcctgcaccaggactggctgaatggca |
| | | | | aggagtacaagtgcaaggtctccaacaaagccctcccagcccccat |
| | | | | cgagaaaaccatctccaaagccaaagggcagccccgagaaccacag |
| | | | | gtgtacaccctgcccccatcccgggatgagctgaccaagaaccagg |
| | | | | tcagcctgacctgcctggtcaaaggcttctatcccagcgacatcgc |
| | | | | cgtggagtgggagagcaatgggcagccggagaacaactacaagacc |
| | | | | acgcctcccgtgctggactccgacggctccttcttcctctacagca |
| | | | | agctcaccgtggacaagagcaggtggcagcaggggaacgtcttctc |
| | | | | atgctccgtgatgcatgaggctctgcacaaccactacacgcagaag |
| | | | | agcctctccctgtctccgggtaaa |
| 205 | | | Heavy Chain | ASTKGPSVFPLAPSSKSTSGGTAALGCLVKDYFPEPVTVSWNSGAL |
| | | | Constant | TSGVHTFPAVLQSSGLYSLSSVVTVPSSSLGTQTYICNVNHKPSNT |
| | | | Region | KVDKKVEPKSCDKTHTCPPCPAPELAGAPSVFLFPPKPKDTLMISR |
| | | | Amino Acid | TPEVTCVVVDVSHEDPEVKFNWYVDGVEVHNAKTKPREEQYNSTYR |
| | | | Sequence | VVSVLTVLHQDWLNGKEYKCKVSNKALPAPIEKTISKAKGQPREPQ |
| | | | (Two | VYTLPPSRDELTKNQVSLTCLVKGFYPSDIAVEWESNGQPENNYKT |
| | | | residues | TPPVLDSDGSFFLYSKLTVDKSRWQQGNVFSCSVMHEALHNHYTQK |
| | | | that differ | SLSLSPGK |
| | | | from the | |
| | | | wild-type | |
| | | | sequence | |
| | | | are | |
| | | | identified | |
| | | | in bold) | |
| 206 | Human Cκ | IGKC* | Cκ Light | cgtacggtggccgctccctccgtgttcatcttcccaccttccgacg |
| | constant | 01 | Chain | agcagctgaagtccggcaccgcttctgtcgtgtgcctgctgaacaa |
| | region | | Constant | cttctacccccgcgaggccaaggtgcagtggaaggtggacaacgcc |
| | | | Region | ctgcagtccggcaactcccaggaatccgtgaccgagcaggactcca |
| | | | Nucleotide | aggacagcacctactccctgtcctccaccctgaccctgtccaaggc |
| | | | Sequence | cgactacgagaagcacaaggtgtacgcctgcgaagtgacccaccag |
| | | | | ggcctgtctagccccgtgaccaagtctttcaaccggggcgagtgt |
| 207 | | | Cκ Light | RTVAAPSVFIFPPSDEQLKSGTASVVCLLNNFYPREAKVQWKVDNA |
| | | | Chain | LQSGNSQESVTEQDSKDSTYSLSSTLTLSKADYEKHKVYACEVTHQ |
| | | | Constant | GLSSPVTKSFNRGEC |
| | | | Region | |
| | | | Amino Acid | |
| | | | Sequence | |
| 208 | Human Cκ | IGKC* | Cκ Light | cgaactgtggctgcaccatctgtcttcatcttcccgccatctgatg |
| | constant | 02 | Chain | agcagttgaaatctggaactgcctctgttgtgtgcctgctgaataa |
| | region | | Constant | cttctatcccagagaggccaaagtacagtggaaggtggataacgcc |
| | | | Region | ctccaatcgggtaactcccaggagagtgtcacagagcaggagagca |
| | | | Nucleotide | aggacagcacctacagcctcagcagcaccctgacgctgagcaaagc |
| | | | Sequence | agactacgagaaacacaaagtctacgccggcgaagtcacccatcag |
| | | | | ggcctgagctcgcccgtcacaaagagcttcaacaggggagagtgt |
| 209 | | | Cκ Light | RTVAAPSVFIFPPSDEQLKSGTASVVCLLNNFYPREAKVQWKVDNA |
| | | | Chain | LQSGNSQESVTEQESKDSTYSLSSTLTLSKADYEKHKVYAGEVTHQ |
| | | | Constant | GLSSPVTKSFNRGEC |
| | | | Region | |
| | | | Amino Acid | |
| | | | Sequence | |
| 210 | Human Cκ | IGKC* | Cκ Light | cgaactgtggctgcaccatctgtcttcatcttcccgccatctgatg |
| | constant | 03 | Chain | agcagttgaaatctggaactgcctctgttgtgtgcctgctgaataa |
| | region | | Constant | cttctatcccagagaggccaaagtacagcggaaggtggataacgcc |
| | | | Region | ctccaatcgggtaactcccaggagagtgtcacagagcaggagagca |
| | | | Nucleotide | aggacagcacctacagcctcagcagcaccctgacgctgagcaaagc |
| | | | Sequence | agactacgagaaacacaaagtctacgcctgcgaagtcacccatcag |
| | | | | ggcctgagctcgcccgtcacaaagagcttcaacaggggagagtgt |
| 211 | | | Cκ Light | RTVAAPSVFIFPPSDEQLKSGTASVVCLLNNFYPREAKVQRKVDNA |
| | | | Chain | LQSGNSQESVTEQESKDSTYSLSSTLTLSKADYEKHKVYACEVTHQ |
| | | | Constant | GLSSPVTKSFNRGEC |
| | | | Region | |
| | | | Amino Acid | |
| | | | Sequence | |
| 212 | Human Cκ | IGKC* | Cκ Light | cgaactgtggctgcaccatctgtcttcatcttcccgccatctgatg |
| | constant | 04 | Chain | agcagttgaaatctggaactgcctctgttgtgtgcctgctgaataa |
| | region | | Constant | cttctatcccagagaggccaaagtacagtggaaggtggataacgcc |
| | | | Region | ctccaatcgggtaactcccaggagagtgtcacagagcaggacagca |
| | | | Nucleotide | aggacagcacctacagcctcagcagcaccctgacgctgagcaaagc |
| | | | Sequence | agactacgagaaacacaaactctacgcctgcgaagtcacccatcag |
| | | | | ggcctgagctcgcccgtcacaaagagcttcaacaggggagagtgt |
| 213 | | | Cκ Light | RTVAAPSVFIFPPSDEQLKSGTASVVCLLNNFYPREAKVQWKVDNA |
| | | | Chain | LQSGNSQESVTEQDSKDSTYSLSSTLTLSKADYEKHKLYACEVTHQ |
| | | | Constant | GLSSPVTKSFNRGEC |
| | | | Region | |
| | | | Amino Acid | |
| | | | Sequence | |
| 214 | Human Cκ | IGKC* | Cκ Light | cgaactgtggctgcaccatctgtcttcatcttcccgccatctgatg |
| | constant | 05 | Chain | agcagttgaaatctggaactgcctctgttgtgtgcctgctgaataa |
| | region | | Constant | cttctatcccagagaggccaaagtacagtggaaggtggataacgcc |
| | | | Region | ctccaatcgggtaactcccaggagagtgtcacagagcaggacagca |
| | | | Nucleotide | aggacagcacctacagcctcagcaacaccctgacgctgagcaaagc |
| | | | Sequence | agactacgagaaacacaaagtctacgcctgcgaagtcacccatcag |
| | | | | ggcctgagctcgcccgtcacaaagagcttcaacaggggagagtgc |
| 215 | | | Cκ Light | RTVAAPSVFIFPPSDEQLKSGTASVVCLLNNFYPREAKVQWKVDNA |
| | | | Chain | LQSGNSQESVTEQDSKDSTYSLSNTLTLSKADYEKHKVYACEVTHQ |
| | | | Constant | GLSSPVTKSFNRGEC |
| | | | Region | |
| | | | Amino Acid | |
| | | | Sequence | |
| 216 | Human Cλ | IGCA1 | Cλ Light | cccaaggccaaccccacggtcactctgttcccgccctcctctgagg |
| | constant | *01 | Chain | agctccaagccaacaaggccacactagtgtgtctgatcagtgactt |
| | region | | Constant | ctacccgggagctgtgacagtggcttggaaggcagatggcagcccc |
| | | | Region | gtcaaggcgggagtggagacgaccaaaccctccaaacagagcaaca |
| | | | Nucleotide | acaagtacgcggccagcagctacctgagcctgacgcccgagcagtg |
| | | | Sequence | gaagtcccacagaagctacagctgccaggtcacgcatgaagggagc |
| | | | | accgtggagaagacagtggcccctacagaatgttca |
| 217 | | | Cλ Light | PKANPTVTLFPPSSEELQANKATLVCLISDFYPGAVTVAWKADGSP |
| | | | Chain | VKAGVETTKPSKQSNNKYAASSYLSLTPEQWKSHRSYSCQVTHEGS |
| | | | Constant | TVEKTVAPTECS |
| | | | Region | |
| | | | Amino Acid | |
| | | | Sequence | |
| 218 | Human Cλ | IGCA1 | Cλ Light | ggtcagcccaaggccaaccccactgtcactctgttcccgccctcct |
| | constant | *02 | Chain | ctgaggagctccaagccaacaaggccacactagtgtgtctgatcag |
| | region | | Constant | tgacttctacccgggagctgtgacagtggcctggaaggcagatggc |
| | | | Region | agccccgtcaaggcgggagtggagaccaccaaaccctccaaacaga |
| | | | Nucleotide | gcaacaacaagtacgcggccagcagctacctgagcctgacgcccga |
| | | | Sequence | gcagtggaagtcccacagaagctacagctgccaggtcacgcatgaa |
| | | | | gggagcaccgtggagaagacagtggcccctacagaatgttca |
| 219 | | | Cλ Light | GQPKANPTVTLFPPSSEELQANKATLVCLISDFYPGAVTVAWKADG |
| | | | Chain | SPVKAGVETTKPSKQSNNKYAASSYLSLTPEQWKSHRSYSCQVTHE |
| | | | Constant | GSTVEKTVAPTECS |
| | | | Region | |
| | | | Amino Acid | |
| | | | Sequence | |
| 220 | Human Cλ | IGCA2 | Cλ Light | ggtcagcccaaggccaaccccactgtcactctgttcccgccctcct |
| | constant | *01 | Chain | ctgaggagctccaagccaacaaggccacactagtgtgtctgatcag |
| | region | | Constant | tgacttctacccgggagctgtgacagtggcctggaaggcagatggc |
| | | | Region | agccccgtcaaggcgggagtggagaccaccaaaccctccaaacaga |
| | | | Nucleotide | gcaacaacaagtacgcggccagcagctacctgagcctgacgcccga |
| | | | Sequence - | gcagtggaagtcccacagaagctacagctgccaggtcacgcatgaa |
| | | | Version A | gggagcaccgtggagaagacagtggcccctacagaatgttca |
| 221 | | | Cλ Light | ggccagcctaaggccgctccttctgtgaccctgttccccccatcct |
| | | | Chain | ccgaggaactgcaggctaacaaggccaccctcgtgtgcctgatcag |
| | | | Constant | cgacttctaccctggcgccgtgaccgtggcctggaaggctgatagc |
| | | | Region | tctcctgtgaaggccggcgtggaaaccaccaccccttccaagcagt |
| | | | Nucleotide | ccaacaacaaatacgccgcctcctcctacctgtccctgacccctga |
| | | | Sequence - | gcagtggaagtcccaccggtcctacagctgccaagtgacccacgag |
| | | | Version B | ggctccaccgtggaaaagaccgtggctcctaccgagtgctcc |
| 222 | | | Cλ Light | ggccagcctaaagctgcccccagcgtcaccctgtttcctccctcca |
| | | | Chain | gcgaggagctccaggccaacaaggccaccctcgtgtgcctgatctc |
| | | | Constant | cgacttctatcccggcgctgtgaccgtggcttggaaagccgactcc |
| | | | Region | agccctgtcaaagccggcgtggagaccaccacaccctccaagcagt |
| | | | Nucleotide | ccaacaacaagtacgccgcctccagctatctctccctgacccctga |
| | | | Sequence - | gcagtggaagtcccaccggtcctactcctgtcaggtgacccacgag |
| | | | Version C | ggctccaccgtggaaaagaccgtcgcccccaccgagtgctcc |
| 223 | | | Cλ Light | GQPKANPTVTLFPPSSEELQANKATLVCLISDFYPGAVTVAWKADG |
| | | | Chain | SPVKAGVETTKPSKQSNNKYAASSYLSLTPEQWKSHRSYSCQVTHE |
| | | | Constant | GSTVEKTVAPTECS |
| | | | Region | |
| | | | Amino Acid | |
| | | | Sequence - | |
| | | | Encoded by | |
| | | | Version A, | |
| | | | B & C | |
| 224 | Human Cλ | IGCA2 | Cλ Light | ggtcagcccaaggctgccccctcggtcactctgttcccgccctcct |
| | constant | *02 & | Chain | ctgaggagcttcaagccaacaaggccacactggtgtgtctcataag |
| | region | IGLC2 | Constant | tgacttctacccgggagccgtgacagtggcctggaaggcagatagc |
| | | *03 | Region | agccccgtcaaggcgggagtggagaccaccacaccctccaaacaaa |
| | | | Nucleotide | gcaacaacaagtacgcggccagcagctatctgagcctgacgcctga |
| | | | Sequence | gcagtggaagtcccacagaagctacagctgccaggtcacgcatgaa |
| | | | | gggagcaccgtggagaagacagtggcccctacagaatgttca |
| 225 | | | Cλ Light | GQPKAAPSVTLFPPSSEELQANKATLVCLISDFYPGAVTVAWKADS |
| | | | Chain | SPVKAGVETTTPSKQSNNKYAASSYLSLTPEQWKSHRSYSCQVTHE |
| | | | Constant | GSTVEKTVAPTECS |
| | | | Region | |
| | | | Amino Acid | |
| | | | Sequence | |
| 226 | Human Cλ | IGCA3 | Cλ Light | cccaaggctgccccctcggtcactctgttcccaccctcctctgagg |
| | constant | *01 | Chain | agcttcaagccaacaaggccacactggtgtgtctcataagtgactt |
| | region | | Constant | ctacccgggagccgtgacagttgcctggaaggcagatagcagcccc |
| | | | Region | gtcaaggcgggggtggagaccaccacaccctccaaacaaagcaaca |
| | | | Nucleotide | acaagtacgcggccagcagctacctgagcctgacgcctgagcagtg |
| | | | Sequence | gaagtcccacaaaagctacagctgccaggtcacgcatgaagggagc |
| | | | | accgtggagaagacagttgcccctacggaatgttca |
| 227 | | | Cλ Light | PKAAPSVTLFPPSSEELQANKATLVCLISDFYPGAVTVAWKADSSP |
| | | | Chain | VKAGVETTTPSKQSNNKYAASSYLSLTPEQWKSHKSYSCQVTHEGS |
| | | | Constant | TVEKTVAPTECS |
| | | | Region | |
| | | | Amino Acid | |
| | | | Sequence | |
| 228 | Human Cλ | IGCA3 | Cλ Light | ggtcagcccaaggctgccccctcggtcactctgttcccaccctcct |
| | constant | *02 | Chain | ctgaggagcttcaagccaacaaggccacactggtgtgtctcataag |
| | region | | Constant | tgacttctacccggggccagtgacagttgcctggaaggcagatagc |
| | | | Region | agccccgtcaaggcgggggtggagaccaccacaccctccaaacaaa |
| | | | Nucleotide | gcaacaacaagtacgcggccagcagctacctgagcctgacgcctga |
| | | | Sequence | gcagtggaagtcccacaaaagctacagctgccaggtcacgcatgaa |
| | | | | gggagcaccgtggagaagacagtggcccctacggaatgttca |
| 229 | | | Cλ Light | GQPKAAPSVTLFPPSSEELQANKATLVCLISDFYPGPVTVAWKADS |
| | | | Chain | SPVKAGVETTTPSKQSNNKYAASSYLSLTPEQWKSHKSYSCQVTHE |
| | | | Constant | GSTVEKTVAPTECS |
| | | | Region | |
| | | | Amino Acid | |
| | | | Sequence | |
| 230 | Human Cλ | IGCA3 | Cλ Light | ggtcagcccaaggctgccccctcggtcactctgttcccaccctcct |
| | constant | *03 | Chain | ctgaggagcttcaagccaacaaggccacactggtgtgtctcataag |
| | region | | Constant | tgacttctacccgggagccgtgacagtggcctggaaggcagatagc |
| | | | Region | agccccgtcaaggcgggagtggagaccaccacaccctccaaacaaa |
| | | | Nucleotide | gcaacaacaagtacgcggccagcagctacctgagcctgacgcctga |
| | | | Sequence | gcagtggaagtcccacaaaagctacagctgccaggtcacgcatgaa |
| | | | | gggagcaccgtggagaagacagtggcccctacagaatgttca |
| 231 | | | Cλ Light | GQPKAAPSVTLFPPSSEELQANKATLVCLISDFYPGAVTVAWKADS |
| | | | Chain | SPVKAGVETTTPSKQSNNKYAASSYLSLTPEQWKSHKSYSCQVTHE |
| | | | Constant | GSTVEKTVAPTECS |
| | | | Region | |
| | | | Amino Acid | |
| | | | Sequence | |
| 232 | Human Cλ | IGCA3 | Cλ Light | ggtcagcccaaggctgccccctcggtcactctgttcccgccctcct |
| | constant | *04 | Chain | ctgaggagcttcaagccaacaaggccacactggtgtgtctcataag |
| | region | | Constant | tgacttctacccgggagccgtgacagtggcctggaaggcagatagc |
| | | | Region | agccccgtcaaggcgggagtggagaccaccacaccctccaaacaaa |
| | | | Nucleotide | gcaacaacaagtacgcggccagcagctacctgagcctgacgcctga |
| | | | Sequence | gcagtggaagtcccacagaagctacagctgccaggtcacgcatgaa |
| | | | | gggagcaccgtggagaagacagtggcccctacagaatgttca |
| 233 | | | Cλ Light | GQPKAAPSVTLFPPSSEELQANKATLVCLISDFYPGAVTVAWKADS |
| | | | Chain | SPVKAGVETTTPSKQSNNKYAASSYLSLTPEQWKSHRSYSCQVTHE |
| | | | Constant | GSTVEKTVAPTECS |
| | | | Region | |
| | | | Amino Acid | |
| | | | Sequence | |
| 234 | Human Cλ | IGCA6 | Cλ Light | ggtcagcccaaggctgccccatcggtcactctgttcccgccctcct |
| | constant | *01 | Chain | ctgaggagcttcaagccaacaaggccacactggtgtgcctgatcag |
| | region | | Constant | tgacttctacccgggagctgtgaaagtggcctggaaggcagatggc |
| | | | Region | agccccgtcaacacgggagtggagaccaccacaccctccaaacaga |
| | | | Nucleotide | gcaacaacaagtacgcggccagcagctacctgagcctgacgcctga |
| | | | Sequence | gcagtggaagtcccacagaagctacagctgccaggtcacgcatgaa |
| | | | | gggagcaccgtggagaagacagtggcccctgcagaatgttca |
| 235 | | | Cλ Light | GQPKAAPSVTLFPPSSEELQANKATLVCLISDFYPGAVKVAWKADG |
| | | | Chain | SPVNTGVETTTPSKQSNNKYAASSYLSLTPEQWKSHRSYSCQVTHE |
| | | | Constant | GSTVEKTVAPAECS |
| | | | Region | |
| | | | Amino Acid | |
| | | | Sequence | |
| 236 | Human Cλ | IGLC7 | Cλ Light | ggtcagcccaaggctgccccatcggtcactctgttcccaccctcct |
| | constant | *01 & | Chain | ctgaggagcttcaagccaacaaggccacactggtgtgtctcgtaag |
| | region | IGCλ7 | Constant | tgacttctacccgggagccgtgacagtggcctggaaggcagatggc |
| | | *02 | Region | agccccgtcaaggtgggagtggagaccaccaaaccctccaaacaaa |
| | | | Nucleotide | gcaacaacaagtatgcggccagcagctacctgagcctgacgcccga |
| | | | Sequence | gcagtggaagtcccacagaagctacagctgccgggtcacgcatgaa |
| | | | | gggagcaccgtggagaagacagtggcccctgcagaatgctct |
| 237 | | | Cλ Light | GQPKAAPSVTLFPPSSEELQANKATLVCLVSDFYPGAVTVAWKADG |
| | | | Chain | SPVKVGVETTKPSKQSNNKYAASSYLSLTPEQWKSHRSYSCRVTHE |
| | | | Constant | GSTVEKTVAPAECS |
| | | | Region | |
| | | | Amino Acid | |
| | | | Sequence | |
| 238 | 413G05 - | Amino acid | | GFTFSDYY |
| | CDRH1 | sequence of CDRH1 | | |
| | (IMGT) | of 413G05 using | | |
| | | IMGT | | |
| 239 | 413G05 - | Amino acid | | ISTSGSTI |
| | CDRH2 | sequence of CDRH2 | | |
| | (IMGT) | of 413G05 using | | |
| | | IMGT | | |
| 240 | 413G05 - | Amino acid | | ARGITGTNFYHYGLGV |
| | CDRH3 | sequence of CDRH3 | | |
| | (IMGT) | of 413G05 using | | |
| | | IMGT | | |
| 241 | 413G05 - | Amino acid | | DYYMS |
| | CDRH1 | sequence of CDRH1 | | |
| | (Kabat) | of 413G05 using | | |
| | | Kabat | | |
| 242 | 413G05 - | Amino acid | | YISTSGSTIYYADSVKG |
| | CDRH2 | sequence of CDRH2 | | |
| | (Kabat) | of 413G05 using | | |
| | | Kabat | | |
| 243 | 413G05 - | Amino acid | | GITGTNFYHYGLGV |
| | CDRH3 | sequence of CDRH3 | | |
| | (Kabat) | of 413G05 using | | |
| | | Kabat | | |
| 244 | 413G05 - | Amino acid | | QVQLVESGGGLVKPGGSLRLSCAASGFTFSDYYMSWIRQVPGKGLE |
| | Heavy | sequence of V_{H} of | | WVSYISTSGSTIYYADSVKGRFTISRDNAKNSLYLQMNSLRAEDAA |
| | chain | 413G05 | | VYHCARGITGTNFYHYGLGVWGQGTTVTVSS |
| | variable | | | |
| | region | | | |
| 245 | 413G05 - | Nucleic acid | | CAGGTGCAGCTGGTGGAGTCTGGGGGAGGCTTGGTCAAGCCTGGAG |
| | Heavy | sequence of V_{H} of | | GGTCCCTGAGACTCTCCTGTGCAGCCTCTGGATTCACCTTCAGTGA |
| | chain | 413G05 | | CTACTACATGAGCTGGATCCGCCAGGTTCCAGGGAAGGGGCTGGAG |
| | variable | | | TGGGTTTCATACATTAGTACTAGTGGTAGTACCATATACTACGCAG |
| | region | | | ACTCTGTGAAGGGCCGATTCACCATCTCCAGGGACAACGCCAAGAA |
| | | | | CTCACTGTATCTACAAATGAACAGCCTGAGAGCCGAGGACGCGGCC |
| | | | | GTGTATCACTGTGCGAGAGGTATAACTGGAACTAACTTCTACCACT |
| | | | | ACGGTTTGGGCGTCTGGGGCCAAGGGACCACGGTCACCGTCTCCTC |
| | | | | AG |
| 246 | 413G05 - | Amino acid | | QVQLVESGGGLVKPGGSLRLSCAASGFTFSDYYMSWIRQVPGKGLE |
| | full | sequence of 413G05 | | WVSYISTSGSTIYYADSVKGRFTISRDNAKNSLYLQMNSLRAEDAA |
| | heavy | heavy chain | | VYHCARGITGTNFYHYGLGVWGQGTTVTVSSASTKGPSVFPLAPSS |
| | chain | | | KSTSGGTAALGCLVKDYFPEPVTVSWNSGALTSGVHTFPAVLQSSG |
| | sequence | | | LYSLSSVVTVPSSSLGTQTYICNVNHKPSNTKVDKKVEPKSCDKTH |
| | | | | TCPPCPAPELLGGPSVFLFPPKPKDTLMISRTPEVTCVVVDVSHED |
| | | | | PEVKFNWYVDGVEVHNAKTKPREEQYNSTYRVVSVLTVLHQDWLNG |
| | | | | KEYKCKVSNKALPAPIEKTISKAKGQPREPQVYTLPPSRDELTKNQ |
| | | | | VSLTCLVKGFYPSDIAVEWESNGQPENNYKTTPPVLDSDGSFFLYS |
| | | | | KLTVDKSRWQQGNVFSCSVMHEALHNHYTQKSLSLSPGK |
| 247 | 413G05 - | Nucleic acid | | CAGGTGCAGCTGGTGGAGTCTGGGGGAGGCTTGGTCAAGCCTGGAG |
| | full | sequence of 413G05 | | GGTCCCTGAGACTCTCCTGTGCAGCCTCTGGATTCACCTTCAGTGA |
| | heavy | heavy chain | | CTACTACATGAGCTGGATCCGCCAGGTTCCAGGGAAGGGGCTGGAG |
| | chain | | | TGGGTTTCATACATTAGTACTAGTGGTAGTACCATATACTACGCAG |
| | sequence | | | ACTCTGTGAAGGGCCGATTCACCATCTCCAGGGACAACGCCAAGAA |
| | | | | CTCACTGTATCTACAAATGAACAGCCTGAGAGCCGAGGACGCGGCC |
| | | | | GTGTATCACTGTGCGAGAGGTATAACTGGAACTAACTTCTACCACT |
| | | | | ACGGTTTGGGCGTCTGGGGCCAAGGGACCACGGTCACCGTCTCCTC |
| | | | | AGCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAGG |
| | | | | ACTACTTCCCCGAGCCTGTGACCGTGTCCTGGAACTCTGGCGCTCT |
| | | | | GACCAGCGGAGTGCACACCTTCCCTGCTGTGCTGCAGTCCTCCGGC |
| | | | | CTGTACTCCCTGTCCTCCGTCGTGACCGTGCCTTCCAGCTCTCTGG |
| | | | | GCACCCAGACCTACATCTGCAACGTGAACCACAAGCCCTCCAACAC |
| | | | | CAAGGTGGACAAGAAGGTGGAACCCAAGTCCTGCGACAAGACCCAC |
| | | | | ACCTGTCCCCCTTGTCCTGCCCCTGAACTGCTGGGCGGACCTTCCG |
| | | | | TGTTCCTGTTCCCCCCAAAGCCCAAGGACACCCTGATGATCTCCCG |
| | | | | GACCCCCGAAGTGACCTGCGTGGTGGTGGATGTGTCCCACGAGGAC |
| | | | | CCTGAAGTGAAGTTCAATTGGTACGTGGACGGCGTGGAAGTGCACA |
| | | | | ACGCCAAGACCAAGCCTAGAGAGGAACAGTACAACTCCACCTACCG |
| | | | | GGTGGTGTCCGTGCTGACCGTGCTGCACCAGGATTGGCTGAACGGC |
| | | | | AAAGAGTACAAGTGCAAGGTGTCCAACAAGGCCCTGCCTGCCCCCA |
| | | | | TCGAAAAGACCATCTCCAAGGCCAAGGGCCAGCCCCGGGAACCCCA |
| | | | | GGTGTACACACTGCCCCCTAGCAGGGACGAGCTGACCAAGAACCAG |
| | | | | GTGTCCCTGACCTGTCTCGTGAAAGGCTTCTACCCCTCCGATATCG |
| | | | | CCGTGGAATGGGAGTCCAACGGCCAGCCTGAGAACAACTACAAGAC |
| | | | | CACCCCCCCTGTGCTGGACTCCGACGGCTCATTCTTCCTGTACAGC |
| | | | | AAGCTGACAGTGGACAAGTCCCGGTGGCAGCAGGGCAACGTGTTCT |
| | | | | CCTGCTCCGTGATGCACGAGGCCCTGCACAACCACTACACCCAGAA |
| | | | | GTCCCTGTCCCTGAGCCCCGGCAAG |
| 248 | 413G05 - | Amino acid | | QGINSW |
| | CDRL1 | sequence of CDRL1 | | |
| | (IMGT) | of 413G05 using | | |
| | | IMGT | | |
| 249 | 413G05 - | Amino acid | | AAS |
| | CDRL2 | sequence of CDRL2 | | |
| | (IMGT) | of 413G05 using | | |
| | | IMGT | | |
| 250 | 413G05 - | Amino acid | | QQVNSFPLT |
| | CDRL3 | sequence of CDRL3 | | |
| | (IMGT) | of 413G05 using | | |
| | | IMGT | | |
| 251 | 413G05 - | Amino acid | | RASQGINSWLA |
| | CDRL1 | sequence of CDRL1 | | |
| | (Kabat) | of 413G05 using | | |
| | | Kabat | | |
| 252 | 413G05 - | Amino acid | | AASTLQS |
| | CDRL2 | sequence of CDRL2 | | |
| | (Kabat) | of 413G05 using | | |
| | | Kabat | | |
| 253 | 413G05 - | Amino acid | | QQVNSFPLT |
| | CDRL3 | sequence of CDRL3 | | |
| | (Kabat) | of 413G05 using | | |
| | | Kabat | | |
| 254 | 413G05 - | Amino acid | | DIQMTQSPSSVSASVGDRVTITCRASQGINSWLAWYQQKPGKAPKL |
| | Light | sequence of V_{L} of | | LIYAASTLQSGVPSRFSGSGSGADFTLTISSLQPEDFATYYCQQVN |
| | chain | 413G05 | | SFPLTFGGGTKVEIK |
| | variable | | | |
| | region | | | |
| 255 | 413G05 - | Nucleic acid | | GACATCCAGATGACCCAGTCTCCATCTTCCGTGTCTGCATCTGTAG |
| | Light | sequence of V_{L} of | | GAGACAGAGTCACCATCACTTGTCGGGCGAGTCAGGGTATTAACAG |
| | chain | 413G05 | | CTGGTTAGCCTGGTATCAGCAGAAACCAGGGAAAGCCCCTAAGCTC |
| | variable | | | CTGATCTATGCTGCATCCACTTTGCAAAGTGGGGTCCCATCAAGGT |
| | region | | | TCAGCGGCAGTGGGTCTGGGGCAGATTTCACTCTCACCATCAGCAG |
| | | | | CCTGCAGCCTGAAGATTTTGCAACTTACTATTGTCAACAGGTTAAC |
| | | | | AGTTTCCCGCTCACTTTCGGCGGAGGGACCAAGGTGGAGATCAAAC |
| 256 | 413G05 - | Amino acid | | DIQMTQSPSSVSASVGDRVTITCRASQGINSWLAWYQQKPGKAPKL |
| | full | sequence of 413G05 | | LIYAASTLQSGVPSRFSGSGSGADFTLTISSLQPEDFATYYCQQVN |
| | light | light chain | | SFPLTFGGGTKVEIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 257 | 413G05 - | Nucleic acid | | GACATCCAGATGACCCAGTCTCCATCTTCCGTGTCTGCATCTGTAG |
| | full | sequence of 413G05 | | GAGACAGAGTCACCATCACTTGTCGGGCGAGTCAGGGTATTAACAG |
| | light | light chain | | CTGGTTAGCCTGGTATCAGCAGAAACCAGGGAAAGCCCCTAAGCTC |
| | chain | | | CTGATCTATGCTGCATCCACTTTGCAAAGTGGGGTCCCATCAAGGT |
| | sequence | | | TCAGCGGCAGTGGGTCTGGGGCAGATTTCACTCTCACCATCAGCAG |
| | | | | CCTGCAGCCTGAAGATTTTGCAACTTACTATTGTCAACAGGTTAAC |
| | | | | AGTTTCCCGCTCACTTTCGGCGGAGGGACCAAGGTGGAGATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 258 | 413F09 - | Amino acid | | GFTFSYYA |
| | CDRH1 | sequence of CDRH1 | | |
| | (IMGT) | of 413F09 using | | |
| | | IMGT | | |
| 259 | 413F09 - | Amino acid | | ISGGGGNT |
| | CDRH2 | sequence of CDRH2 | | |
| | (IMGT) | of 413F09 using | | |
| | | IMGT | | |
| 260 | 413F09 - | Amino acid | | AKDRMKQLVRAYYFDY |
| | CDRH3 | sequence of CDRH3 | | |
| | (IMGT) | of 413F09 using | | |
| | | IMGT | | |
| 261 | 413F09 - | Amino acid | | YYAMS |
| | CDRH1 | sequence of CDRH1 | | |
| | (Kabat) | of 413F09 using | | |
| | | Kabat | | |
| 262 | 413F09 - | Amino acid | | TISGGGGNTHYADSVKG |
| | CDRH2 | sequence of CDRH2 | | |
| | (Kabat) | of 413F09 using | | |
| | | Kabat | | |
| 263 | 413F09 - | Amino acid | | DRMKQLVRAYYFDY |
| | CDRH3 | sequence of CDRH3 | | |
| | (Kabat) | of 413F09 using | | |
| | | Kabat | | |
| 264 | 413F09 - | Amino acid | | EVPLVESGGGLVQPGGSLRLSCAASGFTFSYYAMSWVRQAPGKGLD |
| | Heavy | sequence of V_{H} of | | WVSTISGGGGNTHYADSVKGRFTISRDNSKNTLYLHMNSLRAEDTA |
| | chain | 413F09 | | VYYCAKDRMKQLVRAYYFDYWGQGTLVTVSS |
| | variable | | | |
| | region | | | |
| 265 | 413F09 - | Nucleic acid | | GAGGTGCCGCTGGTGGAGTCTGGGGGAGGCTTGGTACAGCCTGGGG |
| | Heavy | sequence of V_{H} of | | GGTCCCTGAGACTCTCCTGTGCAGCCTCTGGATTCACGTTTAGCTA |
| | chain | 413F09 | | CTATGCCATGAGCTGGGTCCGTCAGGCTCCAGGGAAGGGGCTGGAC |
| | variable | | | TGGGTCTCAACTATTAGTGGTGGTGGTGGTAACACACACTACGCAG |
| | region | | | ACTCCGTGAAGGGCCGATTCACTATATCCAGAGACAATTCCAAGAA |
| | | | | CACGCTGTATCTGCACATGAACAGCCTGAGAGCCGAAGACACGGCC |
| | | | | GTCTATTACTGTGCGAAGGATCGGATGAAACAGCTCGTCCGGGCCT |
| | | | | ACTACTTTGACTACTGGGGCCAGGGAACCCTGGTCACCGTCTCCTC |
| | | | | AG |
| 266 | 413F09 - | Amino acid | | EVPLVESGGGLVQPGGSLRLSCAASGFTFSYYAMSWVRQAPGKGLD |
| | full | sequence of 413F09 | | WVSTISGGGGNTHYADSVKGRFTISRDNSKNTLYLHMNSLRAEDTA |
| | heavy | heavy chain | | VYYCAKDRMKQLVRAYYFDYWGQGTLVTVSSASTKGPSVFPLAPSS |
| | chain | | | KSTSGGTAALGCLVKDYFPEPVTVSWNSGALTSGVHTFPAVLQSSG |
| | sequence | | | LYSLSSVVTVPSSSLGTQTYICNVNHKPSNTKVDKKVEPKSCDKTH |
| | | | | TCPPCPAPELLGGPSVFLFPPKPKDTLMISRTPEVTCVVVDVSHED |
| | | | | PEVKFNWYVDGVEVHNAKTKPREEQYNSTYRVVSVLTVLHQDWLNG |
| | | | | KEYKCKVSNKALPAPIEKTISKAKGQPREPQVYTLPPSRDELTKNQ |
| | | | | VSLTCLVKGFYPSDIAVEWESNGQPENNYKTTPPVLDSDGSFFLYS |
| | | | | KLTVDKSRWQQGNVFSCSVMHEALHNHYTQKSLSLSPGK |
| 267 | 413F09 - | Nucleic acid | | GAGGTGCCGCTGGTGGAGTCTGGGGGAGGCTTGGTACAGCCTGGGG |
| | full | sequence of 413F09 | | GGTCCCTGAGACTCTCCTGTGCAGCCTCTGGATTCACGTTTAGCTA |
| | heavy | heavy chain | | CTATGCCATGAGCTGGGTCCGTCAGGCTCCAGGGAAGGGGCTGGAC |
| | chain | | | TGGGTCTCAACTATTAGTGGTGGTGGTGGTAACACACACTACGCAG |
| | sequence | | | ACTCCGTGAAGGGCCGATTCACTATATCCAGAGACAATTCCAAGAA |
| | | | | CACGCTGTATCTGCACATGAACAGCCTGAGAGCCGAAGACACGGCC |
| | | | | GTCTATTACTGTGCGAAGGATCGGATGAAACAGCTCGTCCGGGCCT |
| | | | | ACTACTTTGACTACTGGGGCCAGGGAACCCTGGTCACCGTCTCCTC |
| | | | | AGCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAGG |
| | | | | ACTACTTCCCCGAGCCTGTGACCGTGTCCTGGAACTCTGGCGCTCT |
| | | | | GACCAGCGGAGTGCACACCTTCCCTGCTGTGCTGCAGTCCTCCGGC |
| | | | | CTGTACTCCCTGTCCTCCGTCGTGACCGTGCCTTCCAGCTCTCTGG |
| | | | | GCACCCAGACCTACATCTGCAACGTGAACCACAAGCCCTCCAACAC |
| | | | | CAAGGTGGACAAGAAGGTGGAACCCAAGTCCTGCGACAAGACCCAC |
| | | | | ACCTGTCCCCCTTGTCCTGCCCCTGAACTGCTGGGCGGACCTTCCG |
| | | | | TGTTCCTGTTCCCCCCAAAGCCCAAGGACACCCTGATGATCTCCCG |
| | | | | GACCCCCGAAGTGACCTGCGTGGTGGTGGATGTGTCCCACGAGGAC |
| | | | | CCTGAAGTGAAGTTCAATTGGTACGTGGACGGCGTGGAAGTGCACA |
| | | | | ACGCCAAGACCAAGCCTAGAGAGGAACAGTACAACTCCACCTACCG |
| | | | | GGTGGTGTCCGTGCTGACCGTGCTGCACCAGGATTGGCTGAACGGC |
| | | | | AAAGAGTACAAGTGCAAGGTGTCCAACAAGGCCCTGCCTGCCCCCA |
| | | | | TCGAAAAGACCATCTCCAAGGCCAAGGGCCAGCCCCGGGAACCCCA |
| | | | | GGTGTACACACTGCCCCCTAGCAGGGACGAGCTGACCAAGAACCAG |
| | | | | GTGTCCCTGACCTGTCTCGTGAAAGGCTTCTACCCCTCCGATATCG |
| | | | | CCGTGGAATGGGAGTCCAACGGCCAGCCTGAGAACAACTACAAGAC |
| | | | | CACCCCCCCTGTGCTGGACTCCGACGGCTCATTCTTCCTGTACAGC |
| | | | | AAGCTGACAGTGGACAAGTCCCGGTGGCAGCAGGGCAACGTGTTCT |
| | | | | CCTGCTCCGTGATGCACGAGGCCCTGCACAACCACTACACCCAGAA |
| | | | | GTCCCTGTCCCTGAGCCCCGGCAAG |
| 268 | 413F09 - | Amino acid | | QDISTY |
| | CDRL1 | sequence of CDRL1 | | |
| | (IMGT) | of 413F09 using | | |
| | | IMGT | | |
| 269 | 413F09 - | Amino acid | | GTS |
| | CDRL2 | sequence of CDRL2 | | |
| | (IMGT) | of 413F09 using | | |
| | | IMGT | | |
| 270 | 413F09 - | Amino acid | | QQLHTDPIT |
| | CDRL3 | sequence of CDRL3 | | |
| | (IMGT) | of 413F09 using | | |
| | | IMGT | | |
| 271 | 413F09 - | Amino acid | | WASQDISTYLG |
| | CDRL1 | sequence of CDRL1 | | |
| | (Kabat) | of 413F09 using | | |
| | | Kabat | | |
| 272 | 413F09 - | Amino acid | | GTSSLQS |
| | CDRL2 | sequence of CDRL2 | | |
| | (Kabat) | of 413F09 using | | |
| | | Kabat | | |
| 273 | 413F09 - | Amino acid | | QQLHTDPIT |
| | CDRL3 | sequence of CDRL3 | | |
| | (Kabat) | of 413F09 using | | |
| | | Kabat | | |
| 274 | 413F09 - | Amino acid | | DIQLTQSPSFLSASVGDRVTITCWASQDISTYLGWYQQKPGKAPKL |
| | Light | sequence of V_{L} of | | LIYGTSSLQSGVPSRFSGSGSGTEFTLTISSLQPEDFATYYCQQLH |
| | chain | 413F09 | | TDPITFGQGTRLEIK |
| | variable | | | |
| | region | | | |
| 275 | 413F09 - | Nucleic acid | | GACATCCAGTTGACCCAGTCTCCATCCTTCCTGTCTGCATCTGTAG |
| | Light | sequence of V_{L} of | | GAGACAGAGTCACCATCACTTGCTGGGCCAGTCAGGACATTAGCAC |
| | chain | 413F09 | | TTATTTAGGCTGGTATCAGCAAAAACCAGGGAAAGCCCCTAAGCTC |
| | variable | | | CTGATCTATGGTACATCCAGTTTGCAAAGTGGGGTCCCATCAAGGT |
| | region | | | TCAGCGGCAGTGGATCTGGGACAGAATTCACTCTCACAATCAGCAG |
| | | | | CCTGCAGCCTGAAGATTTTGCAACTTATTACTGTCAACAGCTTCAT |
| | | | | ACTGACCCGATCACCTTCGGCCAAGGGACACGACTGGAGATCAAAC |
| 276 | 413F09 - | Amino acid | | DIQLTQSPSFLSASVGDRVTITCWASQDISTYLGWYQQKPGKAPKL |
| | full | sequence of 413F09 | | LIYGTSSLQSGVPSRFSGSGSGTEFTLTISSLQPEDFATYYCQQLH |
| | light | light chain | | TDPITFGQGTRLEIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 277 | 413F09 - | Nucleic acid | | GACATCCAGTTGACCCAGTCTCCATCCTTCCTGTCTGCATCTGTAG |
| | full | sequence of 413F09 | | GAGACAGAGTCACCATCACTTGCTGGGCCAGTCAGGACATTAGCAC |
| | light | light chain | | TTATTTAGGCTGGTATCAGCAAAAACCAGGGAAAGCCCCTAAGCTC |
| | chain | | | CTGATCTATGGTACATCCAGTTTGCAAAGTGGGGTCCCATCAAGGT |
| | sequence | | | TCAGCGGCAGTGGATCTGGGACAGAATTCACTCTCACAATCAGCAG |
| | | | | CCTGCAGCCTGAAGATTTTGCAACTTATTACTGTCAACAGCTTCAT |
| | | | | ACTGACCCGATCACCTTCGGCCAAGGGACACGACTGGAGATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 278 | 414B06 - | Amino acid | | GFTFSSYW |
| | CDRH1 | sequence of CDRH1 | | |
| | (IMGT) | of 414B06 using | | |
| | | IMGT | | |
| 279 | 414B06 - | Amino acid | | IKQDGSEK |
| | CDRH2 | sequence of CDRH2 | | |
| | (IMGT) | of 414B06 using | | |
| | | IMGT | | |
| 280 | 414B06 - | Amino acid | | ARVRQWSDYSDY |
| | CDRH3 | sequence of CDRH3 | | |
| | (IMGT) | of 414B06 using | | |
| | | IMGT | | |
| 281 | 414B06 - | Amino acid | | SYWMN |
| | CDRH1 | sequence of CDRH1 | | |
| | (Kabat) | of 414B06 using | | |
| | | Kabat | | |
| 282 | 414B06 - | Amino acid | | NIKQDGSEKYYVDSVKG |
| | CDRH2 | sequence of CDRH2 | | |
| | (Kabat) | of 414B06 using | | |
| | | Kabat | | |
| 283 | 414B06 - | Amino acid | | VRQWSDYSDY |
| | CDRH3 | sequence of CDRH3 | | |
| | (Kabat) | of 414B06 using | | |
| | | Kabat | | |
| 284 | 414B06 - | Amino acid | | EVHLVESGGGLVQPGGSLRLSCAASGFTFSSYWMNWVRQAPGKGLE |
| | Heavy | sequence of V_{H} of | | WVANIKQDGSEKYYVDSVKGRFTVSRDNAKNSLYLQMNSLRAEDTA |
| | chain | 414B06 | | VYYCARVRQWSDYSDYWGQGTPVTVSS |
| | variable | | | |
| | region | | | |
| 285 | 414B06 - | Nucleic acid | | GAGGTGCACCTGGTGGAGTCTGGGGGAGGCTTGGTCCAGCCTGGGG |
| | Heavy | sequence of V_{H} of | | GGTCCCTGAGACTCTCCTGTGCAGCCTCTGGATTCACCTTTAGTAG |
| | chain | 414B06 | | CTATTGGATGAACTGGGTCCGCCAGGCTCCAGGGAAGGGGCTGGAG |
| | variable | | | TGGGTGGCCAACATAAAGCAAGATGGAAGTGAGAAATACTATGTGG |
| | region | | | ACTCTGTGAAGGGCCGCTTCACCGTCTCCAGAGACAACGCCAAGAA |
| | | | | CTCACTGTATCTGCAAATGAACAGCCTGAGAGCCGAGGACACGGCT |
| | | | | GTGTATTACTGTGCGAGAGTTCGACAATGGTCCGACTACTCTGACT |
| | | | | ACTGGGGCCAGGGAACCCCGGTCACCGTCTCCTCAG |
| 286 | 414B06 - | Amino acid | | EVHLVESGGGLVQPGGSLRLSCAASGFTFSSYWMNWVRQAPGKGLE |
| | full | sequence of 414B06 | | WVANIKQDGSEKYYVDSVKGRFTVSRDNAKNSLYLQMNSLRAEDTA |
| | heavy | heavy chain | | VYYCARVRQWSDYSDYWGQGTPVTVSSASTKGPSVFPLAPSSKSTS |
| | chain | | | GGTAALGCLVKDYFPEPVTVSWNSGALTSGVHTFPAVLQSSGLYSL |
| | sequence | | | SSVVTVPSSSLGTQTYICNVNHKPSNTKVDKKVEPKSCDKTHTCPP |
| | | | | CPAPELLGGPSVFLFPPKPKDTLMISRTPEVTCVVVDVSHEDPEVK |
| | | | | FNWYVDGVEVHNAKTKPREEQYNSTYRVVSVLTVLHQDWLNGKEYK |
| | | | | CKVSNKALPAPIEKTISKAKGQPREPQVYTLPPSRDELTKNQVSLT |
| | | | | CLVKGFYPSDIAVEWESNGQPENNYKTTPPVLDSDGSFFLYSKLTV |
| | | | | DKSRWQQGNVFSCSVMHEALHNHYTQKSLSLSPGK |
| 287 | 414B06 - | Nucleic acid | | GAGGTGCACCTGGTGGAGTCTGGGGGAGGCTTGGTCCAGCCTGGGG |
| | full | sequence of 414B06 | | GGTCCCTGAGACTCTCCTGTGCAGCCTCTGGATTCACCTTTAGTAG |
| | heavy | heavy chain | | CTATTGGATGAACTGGGTCCGCCAGGCTCCAGGGAAGGGGCTGGAG |
| | chain | | | TGGGTGGCCAACATAAAGCAAGATGGAAGTGAGAAATACTATGTGG |
| | sequence | | | ACTCTGTGAAGGGCCGCTTCACCGTCTCCAGAGACAACGCCAAGAA |
| | | | | CTCACTGTATCTGCAAATGAACAGCCTGAGAGCCGAGGACACGGCT |
| | | | | GTGTATTACTGTGCGAGAGTTCGACAATGGTCCGACTACTCTGACT |
| | | | | ACTGGGGCCAGGGAACCCCGGTCACCGTCTCCTCAGCCAGCACCAA |
| | | | | GGGCCCCTCTGTGTTCCCTCTGGCCCCTTCCAGCAAGTCCACCTCT |
| | | | | GGCGGAACAGCCGCTCTGGGCTGCCTCGTGAAGGACTACTTCCCCG |
| | | | | AGCCTGTGACCGTGTCCTGGAACTCTGGCGCTCTGACCAGCGGAGT |
| | | | | GCACACCTTCCCTGCTGTGCTGCAGTCCTCCGGCCTGTACTCCCTG |
| | | | | TCCTCCGTCGTGACCGTGCCTTCCAGCTCTCTGGGCACCCAGACCT |
| | | | | ACATCTGCAACGTGAACCACAAGCCCTCCAACACCAAGGTGGACAA |
| | | | | GAAGGTGGAACCCAAGTCCTGCGACAAGACCCACACCTGTCCCCCT |
| | | | | TGTCCTGCCCCTGAACTGCTGGGCGGACCTTCCGTGTTCCTGTTCC |
| | | | | CCCCAAAGCCCAAGGACACCCTGATGATCTCCCGGACCCCCGAAGT |
| | | | | GACCTGCGTGGTGGTGGATGTGTCCCACGAGGACCCTGAAGTGAAG |
| | | | | TTCAATTGGTACGTGGACGGCGTGGAAGTGCACAACGCCAAGACCA |
| | | | | AGCCTAGAGAGGAACAGTACAACTCCACCTACCGGGTGGTGTCCGT |
| | | | | GCTGACCGTGCTGCACCAGGATTGGCTGAACGGCAAAGAGTACAAG |
| | | | | TGCAAGGTGTCCAACAAGGCCCTGCCTGCCCCCATCGAAAAGACCA |
| | | | | TCTCCAAGGCCAAGGGCCAGCCCCGGGAACCCCAGGTGTACACACT |
| | | | | GCCCCCTAGCAGGGACGAGCTGACCAAGAACCAGGTGTCCCTGACC |
| | | | | TGTCTCGTGAAAGGCTTCTACCCCTCCGATATCGCCGTGGAATGGG |
| | | | | AGTCCAACGGCCAGCCTGAGAACAACTACAAGACCACCCCCCCTGT |
| | | | | GCTGGACTCCGACGGCTCATTCTTCCTGTACAGCAAGCTGACAGTG |
| | | | | GACAAGTCCCGGTGGCAGCAGGGCAACGTGTTCTCCTGCTCCGTGA |
| | | | | TGCACGAGGCCCTGCACAACCACTACACCCAGAAGTCCCTGTCCCT |
| | | | | GAGCCCCGGCAAG |
| 288 | 414B06 - | Amino acid | | QGISSW |
| | CDRL1 | sequence of CDRL1 | | |
| | (IMGT) | of 414B06 using | | |
| | | IMGT | | |
| 289 | 414B06 - | Amino acid | | AAS |
| | CDRL2 | sequence of CDRL2 | | |
| | (IMGT) | of 414B06 using | | |
| | | IMGT | | |
| 290 | 414B06 - | Amino acid | | QQANSFPFT |
| | CDRL3 | sequence of CDRL3 | | |
| | (IMGT) | of 414B06 using | | |
| | | IMGT | | |
| 291 | 414B06 - | Amino acid | | RASQGISSWLA |
| | CDRL1 | sequence of CDRL1 | | |
| | (Kabat) | of 414B06 using | | |
| | | Kabat | | |
| 292 | 414B06 - | Amino acid | | AASSLQS |
| | CDRL2 | sequence of CDRL2 | | |
| | (Kabat) | of 414B06 using | | |
| | | Kabat | | |
| 293 | 414B06 - | Amino acid | | QQANSFPFT |
| | CDRL3 | sequence of CDRL3 | | |
| | (Kabat) | of 414B06 using | | |
| | | Kabat | | |
| 294 | 414B06 - | Amino acid | | DIQMTQSPSSVSASVGDRVTITCRASQGISSWLAWYQQKPGKAPKL |
| | Light | sequence of V_{L} of | | LIYAASSLQSGVPSRFSGSGSGTDFTLTISSLQPEDFATYYCQQAN |
| | chain | 414B06 | | SFPFTFGPGTKVDIK |
| | variable | | | |
| | region | | | |
| 295 | 414B06 - | Nucleic acid | | GACATCCAGATGACCCAGTCTCCATCTTCCGTGTCTGCATCTGTAG |
| | Light | sequence of V_{L} of | | GAGACAGAGTCACCATCACTTGTCGGGCGAGTCAGGGTATTAGCAG |
| | chain | 414B06 | | CTGGTTAGCCTGGTATCAGCAGAAACCAGGGAAAGCCCCTAAGCTC |
| | variable | | | CTGATCTATGCTGCATCCAGTTTGCAAAGTGGGGTCCCATCAAGGT |
| | region | | | TCAGCGGCAGTGGATCTGGGACAGATTTCACTCTCACCATCAGCAG |
| | | | | CCTGCAGCCTGAAGATTTTGCAACTTACTATTGTCAACAGGCTAAC |
| | | | | AGTTTCCCATTCACTTTCGGCCCTGGGACCAAAGTGGATATCAAAC |
| 296 | 414B06 - | Amino acid | | DIQMTQSPSSVSASVGDRVTITCRASQGISSWLAWYQQKPGKAPKL |
| | full | sequence of 414B06 | | LIYAASSLQSGVPSRFSGSGSGTDFTLTISSLQPEDFATYYCQQAN |
| | light | light chain | | SFPFTFGPGTKVDIKRTVAAPSVFIFPPSDEQLKSGTASVVCLLNN |
| | chain | | | FYPREAKVQWKVDNALQSGNSQESVTEQDSKDSTYSLSSTLTLSKA |
| | sequence | | | DYEKHKVYACEVTHQGLSSPVTKSFNRGEC |
| 297 | 414B06 - | Nucleic acid | | GACATCCAGATGACCCAGTCTCCATCTTCCGTGTCTGCATCTGTAG |
| | full | sequence of 414B06 | | GAGACAGAGTCACCATCACTTGTCGGGCGAGTCAGGGTATTAGCAG |
| | light | light chain | | CTGGTTAGCCTGGTATCAGCAGAAACCAGGGAAAGCCCCTAAGCTC |
| | chain | | | CTGATCTATGCTGCATCCAGTTTGCAAAGTGGGGTCCCATCAAGGT |
| | sequence | | | TCAGCGGCAGTGGATCTGGGACAGATTTCACTCTCACCATCAGCAG |
| | | | | CCTGCAGCCTGAAGATTTTGCAACTTACTATTGTCAACAGGCTAAC |
| | | | | AGTTTCCCATTCACTTTCGGCCCTGGGACCAAAGTGGATATCAAAC |
| | | | | GTACGGTGGCCGCTCCCTCCGTGTTCATCTTCCCACCTTCCGACGA |
| | | | | GCAGCTGAAGTCCGGCACCGCTTCTGTCGTGTGCCTGCTGAACAAC |
| | | | | TTCTACCCCCGCGAGGCCAAGGTGCAGTGGAAGGTGGACAACGCCC |
| | | | | TGCAGTCCGGCAACTCCCAGGAATCCGTGACCGAGCAGGACTCCAA |
| | | | | GGACAGCACCTACTCCCTGTCCTCCACCCTGACCCTGTCCAAGGCC |
| | | | | GACTACGAGAAGCACAAGGTGTACGCCTGCGAAGTGACCCACCAGG |
| | | | | GCCTGTCTAGCCCCGTGACCAAGTCTTTCAACCGGGGCGAGTGT |
| 298 | Mutated | Amino acid | | DIQMTQSPSSLSASVGDRVTITCRASQSISSYLNWYQQKPGKAPKL |
| | 1D05 - | sequence of 1D05 | | LIYYASSLQSGVPSRFSGSGSGTDFTLTISSLQPEDFATYYCQQSY |
| | LC | kappa light chain | | STPITFGQGTRLEIKRTVAAPSVFIFPPSDEQLKSGTASVVCLLNN |
| | mutant 3 | with V to Y | | FYPREAKVQWKVDNALQSGNSQESVTEQDSKDSTYSLSSTLTLSKA |
| | | mutation in CDRL2 | | DYEKHKVYACEVTHQGLSSPVTKSFNRGEC |
| | | highlighted | | |
| 299 | 1D05 - | Amino acid | | EVQLVESGGGLVQPGRSLRLSCAASGFTFDDYAMHWVRQVPGKGLE |
| | heavy | sequence of IgG1 | | WVSGISWIRTGIGYADSVKGRFTIFRDNAKNSLYLQMNSLRAEDTA |
| | chain | disabled variant | | LYYCAKDMKGSGTYGGWFDTWGQGTLVTVSSASTKGPSVFPLAPSS |
| | disabled | of 1D05 | | KSTSGGTAALGCLVKDYFPEPVTVSWNSGALTSGVHTFPAVLQSSG |
| | IgG1 Fc | | | LYSLSSVVTVPSSSLGTQTYICNVNHKPSNTKVDKKVEPKSCDKTH |
| | | | | TCPPCPAPELAGAPSVFLFPPKPKDTLMISRTPEVTCVVVDVSHED |
| | | | | PEVKFNWYVDGVEVHNAKTKPREEQYNSTYRVVSVLTVLHQDWLNG |
| | | | | KEYKCKVSNKALPAPIEKTISKAKGQPREPQVYTLPPSRDELTKNQ |
| | | | | VSLTCLVKGFYPSDIAVEWESNGQPENNYKTTPPVLDSDGSFFLYS |
| | | | | KLTVDKSRWQQGNVFSCSVMHEALHNHYTQKSLSLSPGK |
| 300 | 1D05 - | 1D05 Light chain | | DIQMTQSPSSLSASVGDRVTITCRASQSISSYLNWYQQKPGKAPKL |
| | light | sequence fused to | | LIYVASSLQSGVPSRFSGSGSGTDFTLTISSLQPEDFATYYCQQSY |
| | chain | wild-type human | | STPITFGQGTRLEIKRTVAAPSVFIFPPSDEQLKSGTASVVCLLNN |
| | IL-2 | IL-2 sequence (IL- | | FYPREAKVQWKVDNALQSGNSQESVTEQDSKDSTYSLSSTLTLSKA |
| | fusion | 2 amino acid | | DYEKHKVYACEVTHQGLSSPVTKSFNRGECAPTSSSTKETQLQLEH |
| | | sequence is | | LLLDLQMILNGINNYKNPKLTRMLTFKFYMPKKATELKHLQCLEEE |
| | | underlined and | | LKPLEEVLNLAQSKNFHLRPRDLISNINVIVLELKGSETTFMCEYA |
| | | region to be | | DETATIVEFLNRWITFCQSIISTLT |
| | | varied is shown in | | |
| | | bold) | | |
| 301 | Human | Uniprot number: | | APTSSSTKETQLQLEHLLLDLQMILNGINNYKNPKLTRMLTFKFYM |
| | IL-2 | P60568 | | PKKATELKHLQCLEEELKPLEEVLNLAQSKNFHLRPRDLISNINVI |
| | | Full length amino | | VLELKGSETTFMCEYADETATIVEFLNRWITFCQSIISTLT |
| | | acid sequence of | | |
| | | human IL-2 (minus | | |
| | | signal sequence) | | |
| 302 | Control | Heavy chain 1D05 | | EVQLVESGGGLVQPGRSLRLSCAASGFTFDDYAMHWVRQVPGKGLE |
| | 1D05 | IgG1 variant fused | | WVSGISWIRTGIGYADSVKGRFTIFRDNAKNSLYLQMNSLRAEDTA |
| | immuno- | at the N-terminus | | LYYCAKDMKGSGTYGGWFDTWGQGTLVTVSSASTKGPSVFPLAPSS |
| | cytokine | to wild-type human | | KSTSGGTAALGCLVKDYFPEPVTVSWNSGALTSGVHTFPAVLQSSG |
| | HC C- | IL2 sequence | | LYSLSSVVTVPSSSLGTQTYICNVNHKPSNTKVDKKVEPKSCDKTH |
| | terminal | (control) | | TCPPCPAPELAGAPSVFLFPPKPKDTLMISRTPEVTCVVVDVSHED |
| | fusion | | | PEVKFNWYVDGVEVHNAKTKPREEQYNSTYRVVSVLTVLHQDWLNG |
| | | | | KEYKCKVSNKALPAPIEKTISKAKGQPREPQVYTLPPSRDELTKNQ |
| | | | | VSLTCLVKGFYPSDIAVEWESNGQPENNYKTTPPVLDSDGSFFLYS |
| | | | | KLTVDKSRWQQGNVFSCSVMHEALHNHYTQKSLSLSPGKAPTSSST |
| | | | | KKTQLQLEHLLLDLQMILNGINNYKNPKLTRMLTFKFYMPKKATEL |
| | | | | KHLQCLEEELKPLEEVLNLAQSKNFHLRPRDLISNINVIVLELKGS |
| | | | | ETTFMCEYADETATIVEFLNRWITFCQSIISTLT |
| 303 | IL-2 D5- | IL-2 IC45 (Del 5- | | APTSTQLQLELLLD |
| | 9 | 9) N terminal IL-2 | | |
| | | sequence | | |
| 304 | IL-2 D1- | IL-2 IC46 (Del 1- | | TQLQLEHLLLD |
| | 9 | 9) N terminal IL-2 | | |
| | | sequence | | |
| 305 | IL-2 D5- | IL-2 IC64 (Del 5- | | APTSKKTQLQLEHLLLD |
| | 7 | 7) N terminal IL-2 | | |
| | | sequence | | |
| 306 | IL-2 D1 | IL-2 D1 N terminal | | PTSSSTKKTQLQLEHLLLD |
| | | IL-2 sequence | | |
| 307 | IL-2 D1- | IL-2 D1-2 N | | TSSSTKKTQLQLEHLLLD |
| | 2 | terminal IL-2 | | |
| | | sequence | | |
| 308 | IL-2 D1- | IL-2 D1-3 N | | SSSTKKTQLQLEHLLLD |
| | 3 | terminal IL-2 | | |
| | | sequence | | |
| 309 | IL-2 D1- | IL-2 D1-4 N | | SSTKKTQLQLEHLLLD |
| | 4 | terminal IL-2 | | |
| | | sequence | | |
| 310 | IL-2 D1- | IL-2 D1-5 N | | STKKTQLQLEHLLLD |
| | 5 | terminal IL-2 | | |
| | | sequence | | |
| 311 | IL-2 D1- | IL-2 D1-6 N | | TKKTQLQLEHLLLD |
| | 6 | terminal IL-2 | | |
| | | sequence | | |
| 312 | IL-2 D1- | IL-2 D1-7 N | | KKTQLQLEHLLLD |
| | 7 | terminal IL-2 | | |
| | | sequence | | |
| 313 | IL-2 D1- | IL-2 D1-8 N | | KTQLQLEHLLLD |
| | 8 | terminal IL-2 | | |
| | | sequence | | |
| 314 | IL-2 D9 | IL-2 D9 N terminal | | APTSSSTKTQLQLEHLLLD |
| | | IL-2 sequence | | |
| 315 | IL-2 D9- | IL-2 D9-8 N | | APTSSSTTQLQLEHLLLD |
| | 8 | terminal IL-2 | | |
| | | sequence | | |
| 316 | IL-2 D9- | IL-2 D9-7 N | | APTSSSTQLQLEHLLLD |
| | 7 | terminal IL-2 | | |
| | | sequence | | |
| 317 | IL-2 D9- | IL-2 D9-6 N | | APTSSTQLQLEHLLLD |
| | 6 | terminal IL-2 | | |
| | | sequence | | |
| 318 | IL-2 D9- | IL-2 D9-4 N | | APTTQLQLEHLLLD |
| | 4 | terminal IL-2 | | |
| | | sequence | | |
| 319 | IL-2 D9- | IL-2 D9-3 N | | APTQLQLEHLLLD |
| | 3 | terminal IL-2 | | |
| | | sequence | | |
| 320 | IL-2 D9- | IL-2 D9-2 N | | ATQLQLEHLLLD |
| | 2 | terminal IL-2 | | |
| | | sequence | | |
| 321 | IL-2 D2- | IL-2 D2-6 N | | ATKKTQLQLEHLLLD |
| | 6 | terminal IL-2 | | |
| | | sequence | | |
| 322 | IL-2 D3- | IL-2 D3-7 N | | APKKTQLQLEHLLLD |
| | 7 | terminal IL-2 | | |
| | | sequence | | |
| 323 | IL-2 D4- | IL-2 D4-8 N | | APTKTQLQLEHLLLD |
| | 8 | terminal IL-2 | | |
| | | sequence | | |
| 324 | C- | Amino acids 21 to | | LQMILNGINNYKNPKLTRMLTFKFYMPKKATELKHLQCLEEELKPL |
| | terminal | 133 of hIL-2 | | EEVLNLAQSKNFHLRPRDLISNINVIVLELKGSETTFMCEYADETA |
| | amino | | | TIVEFLNRWITFCQSIISTLT |
| | acid | | | |
| | sequence | | | |
| | of hIL-2 | | | |
| 325 | Mouse | Uniprot number: | | MRIFAGIIFTACCHLLRAFTITAPKDLYVVEYGSNVTMECRFPVER |
| | PD-L1 | Q9EP73 | | ELDLLALVVYWEKEDEQVIQFVAGEEDLKPQHSNFRGRASLPKDQL |
| | | (ECD highlighted | | LKGNAALQITDVKLQDAGVYCCIISYGGADYKRITLKVNAPYRKIN |
| | | in BOLD, and | | QRISVDPATSEHELICQAEGYPEAEVIWTNSDHQPVSGKRSVTTSR |
| | | cytoplasmic domain | | TEGMLLNVTSSLRVNATANDVFYCTFWRSQPGQNHTAELIIPELPA |
| | | underlined) | | THPPQNRTHWVLLGSILLFLIVVSTVLLFLRKQVRMLDVEKCGVED |
| | | | | TSSKNRNDTQFEET |
| 326 | Mouse | Mouse PD-L1 | | FTITAPKDLYVVEYGSNVTMECRFPVERELDLLALVVYWEKEDEQV |
| | PD-L1 | extracellular | | IQFVAGEEDLKPQHSNFRGRASLPKDQLLKGNAALQITDVKLQDAG |
| | ECD His | domain with his | | VYCCIISYGGADYKRITLKVNAPYRKINQRISVDPATSEHELICQA |
| | | tag | | EGYPEAEVIWTNSDHQPVSGKRSVTTSRTEGMLLNVTSSLRVNATA |
| | | | | NDVFYCTFWRSQPGQNHTAELIIPELPATHPPQNRTHHHHHH |
| 327 | Human | Human IL-2 | | ELCDDDPPEIPHATFKAMAYKEGTMLNCECKRGFRRIKSGSLYMLC |
| | IL-2Rα | receptor alpha | | TGNSSHSSWDNQCQCTSSATRNTTKQVTPQPEEQKERKTTEMQSPM |
| | chain | chain | | QPVDQASLPGHCREPPPWENEATERIYHFVVGQMVYYQCVQGYRAL |
| | | | | HRGPAESVCKMTHGKTRWTQPQLICTGEMETSQFPGEEKPQASPEG |
| | | | | RPESETSCLVTTTDFQIQTEMAATMETSIFTTEYQVAVAGCVFLLI |
| | | | | SVLLLSGLTWQRRQRKSRRTI |
| 328 | Human | Human IL-2 | | AVNGTSQFTCFYNSRANISCVWSQDGALQDTSCQVHAWPDRRRWNQ |
| | IL-2Rβ | receptor beta | | TCELLPVSQASWACNLILGAPDSQKLTTVDIVTLRVLCREGVRWRV |
| | chain | chain | | MAIQDFKPFENLRLMAPISLQVVHVETHRCNISWEISQASHYFERH |
| | | | | LEFEARTLSPGHTWEEAPLLTLKQKQEWICLETLTPDTQYEFQVRV |
| | | | | KPLQGEFTTWSPWSQPLAFRTKPAALGKDTIPWLGHLLVGLSGAFG |
| | | | | FIILVYLLINCRNTGPWLKKVLKCNTPDPSKFFSQLSSEHGGDVQK |
| | | | | WLSSPFPSSSFSPGGLAPEISPLEVLERDKVTQLLLQQDKVPEPAS |
| | | | | LSSNHSLTSCFTNQGYFFFHLPDALEIEACQVYFTYDPYSEEDPDE |
| | | | | GVAGAPTGSSPQPLQPLSGEDDAYCTFPSRDDLLLFSPSLLGGPSP |
| | | | | PSTAPGGSGAGEERMPPSLQERVPRDWDPQPLGPPTPGVPDLVDFQ |
| | | | | PPPELVLREAGEEVPDAGPREGVSFPWSRPPGQGEFRALNARLPLN |
| | | | | TDAYLSLQELQGQDPTHLV |
| 329 | Human | Human IL-2 | | LNTTILTPNGNEDTTADFFLTTMPTDSLSVSTLPLPEVQCFVFNVE |
| | IL-2Rγ | receptor common | | YMNCTWNSSSEPQPTNLTLHYWYKNSDNDKVQKCSHYLFSEEITSG |
| | chain | gamma chain | | CQLQKKEIHLYQTFVVQLQDPREPRRQATQMLKLQNLVIPWAPENL |
| | | | | TLHKLSESQLELNWNNRFLNHCLEHLVQYRTDWDHSWTEQSVDYRH |
| | | | | KFSLPSVDGQKRYTFRVRSRFNPLCGSAQHWSEWSHPIHWGSNTSK |
| | | | | ENPFLFALEAVVISVGSMGLIISLLCVYFWLERTMPRIPTLKNLED |
| | | | | LVTEYHGNFSAWSGVSKGLAESLQPDYSERLCLVSEIPPKGGALGE |
| | | | | GPGASPCNQHSPYWAPPCYTLKPET |
| 330 | IL-7 | Human IL-7 amino | | DCDIEGKDGKQYESVLMVSIDQLLDSMKEIGSNCLNNEFNFFKRHI |
| | | acid sequence | | CDANKEGMFLFRAARKLRQFLKMNSTGDFDLHLLKVSEGTTILLNC |
| | | | | TGQVKGRKPAALGEAQPTKSLEENKSLKEQKKLNDLCFLKRLLQEI |
| | | | | KTCWNKILMGTKEH |
| 331 | IL-15 | Human IL-15 amino | | GIHVFILGCFSAGLPKTEANWVNVISDLKKIEDLIQSMHIDATLYT |
| | | acid sequence | | ESDVHPSCKVTAMKCFLLELQVISLESGDASIHDTVENLIILANNS |
| | | | | LSSNGNVTESGCKECEELEEKNIKEFLQSFVHIVQMFINTS |
| 332 | IL-21 | Human IL-21 amino | | QGQDRHMIRMRQLIDIVDQLKNYVNDLVPEFLPAPEDVETNCEWSA |
| | | acid sequence | | FSCFQKAQLKSANTGNNERIINVSIKKLKRKPPSTNAGRRQKHRLT |
| | | | | CPSCDSYEKKPPKEFLERFKSLLQKMIHQHLSSRTHGSEDS |
| 333 | GM-CSF | Human GM-CSF amino | | APARSPSPSTQPWEHVNAIQEARRLLNLSRDTAAEMNETVEVISEM |
| | | acid sequence | | FDLQEPTCLQTRLELYKQGLRGSLTKLKGPLTMMASHYKQHCPPTP |
| | | | | ETSCATQIITFESFKENLKDFLLVIPFDCWEPVQE |
| 334 | IFNα | Human IFN-α amino | | CDLPQNHGLLSRNTLVLLHQMRRISPFLCLKDRRDFRFPQEMVKGS |
| | | acid sequence | | QLQKAHVMSVLHEMLQQIFSLFHTERSSAAWNMTLLDQLHTELHQQ |
| | | | | LQHLETCLLQVVGEGESAGAISSPALTLRRYFQGIRVYLKEKKYSD |
| | | | | CAWEVVRMEIMKSLFLSTNMQERLRSKDRDLGS |
| 335 | TNFα | Extracellular | | GPQREEFPRDLSLISPLAQAVRSSSRTPSDKPVAHVVANPQAEGQL |
| | | portion of human | | QWLNRRANALLANGVELRDNQLVVPSEGLYLIYSQVLFKGQGCPST |
| | | TNF-α amino acid | | HVLLTHTISRIAVSYQTKVNLLSAIKSPCQRETPEGAEAKPWYEPI |
| | | sequence | | YLGGVFQLEKGDRLSAEINRPDYLDFAESGQVYFGIIAL |
| 336 | IL-12α | Alpha chain of | | RNLPVATPDPGMFPCLHHSQNLLRAVSNMLQKARQTLEFYPCTSEE |
| | | human IL-12 amino | | IDHEDITKDKTSTVEACLPLELTKNESCLNSRETSFITNGSCLASR |
| | | acid sequence | | KTSFMMALCLSSIYEDLKMYQVEFKTMNAKLLMDPKRQIFLDQNML |
| | | | | AVIDELMQALNFNSETVPQKSSLEEPDFYKTKIKLCILLHAFRIRA |
| | | | | VTIDRVMSYLNAS |
| 337 | IL-12β | Beta chain of | | IWELKKDVYVVELDWYPDAPGEMVVLTCDTPEEDGITWTLDQSSEV |
| | | human IL-12 amino | | LGSGKTLTIQVKEFGDAGQYTCHKGGEVLSHSLLLLHKKEDGIWST |
| | | acid sequence | | DILKDQKEPKNKTFLRCEAKNYSGRFTCWWLTTISTDLTFSVKSSR |
| | | | | GSSDPQGVTCGAATLSAERVRGDNKEYEYSVECQEDSACPAAEESL |
| | | | | PIEVMVDAVHKLKYENYTSSFFIRDIIKPDPPKNLQLKPLKNSRQV |
| | | | | EVSWEYPDTWSTPHSYFSLTFCVQVQGKSKREKKDRVFTDKTSATV |
| | | | | ICRKNASISVRAQDRYYSSSWSEWASVPCS |
| 338 | CXCL9 | Human CXCL-9 amino | | TPVVRKGRCSCISTNQGTIHLQSLKDLKQFAPSPSCEKIETIATLK |
| | | acid sequence | | NGVQTCLNPDSADVKELIKKWEKQVSQKKKQKNGKKHQKKKVLKVR |
| | | | | KSQRSRQKKTT |
| 339 | CXCL10 | Human CXCL-10 | | VPLSRTVRCTCISISNQPVNPRSLEKLEIIPASQFCPRVEIIATMK |
| | | amino acid | | KKGEKRCLNPESKAIKNLLKAVSKERSKRSP |
| | | sequence | | |
| 340 | Human WT | IGHG1 | WT human | ASTKGPSVFPLAPSSKSTSGGTAALGCLVKDYFPEPVTVSWNSGAL |
| | IgG1 | *01 & | IgG1 amino | TSGVHTFPAVLQSSGLYSLSSVVTVPSSSLGTQTYICNVNHKPSNT |
| | constant | IGHG1 | acid | KVDKKVEPKSCDKTHTCPPCPAPELLGGPSVFLFPPKPKDTLMISR |
| | region | *02 & | sequence | TPEVTCVVVDVSHEDPEVKFNWYVDGVEVHNAKTKPREEQYNSTYR |
| | | IGHG1 | | VVSVLTVLHQDWLNGKEYKCKVSNKALPAPIEKTISKAKGQPREPQ |
| | | *05 | | VYTLPPSRDELTKNQVSLTCLVKGFYPSDIAVEWESNGQPENNYKT |
| | | (IgG1) | | TPPVLDSDGSFFLYSKLTVDKSRWQQGNVFSCSVMHEALHNHYTQK |
| | | | | SLSLSPGK |
| 341 | | | WT human | GCCAGCACCAAGGGCCCCTCTGTGTTCCCTCTGGCCCCTTCCAGCA |
| | | | IgG1 | AGTCCACCTCTGGCGGAACAGCCGCTCTGGGCTGCCTCGTGAAGGA |
| | | | nucleic | CTACTTCCCCGAGCCTGTGACCGTGTCCTGGAACTCTGGCGCTCTG |
| | | | acid | ACCAGCGGAGTGCACACCTTCCCTGCTGTGCTGCAGTCCTCCGGCC |
| | | | sequence | TGTACTCCCTGTCCTCCGTCGTGACCGTGCCTTCCAGCTCTCTGGG |
| | | | | CACCCAGACCTACATCTGCAACGTGAACCACAAGCCCTCCAACACC |
| | | | | AAGGTGGACAAGAAGGTGGAACCCAAGTCCTGCGACAAGACCCACA |
| | | | | CCTGTCCCCCTTGTCCTGCCCCTGAACTGCTGGGCGGACCTTCCGT |
| | | | | GTTCCTGTTCCCCCCAAAGCCCAAGGACACCCTGATGATCTCCCGG |
| | | | | ACCCCCGAAGTGACCTGCGTGGTGGTGGATGTGTCCCACGAGGACC |
| | | | | CTGAAGTGAAGTTCAATTGGTACGTGGACGGCGTGGAAGTGCACAA |
| | | | | CGCCAAGACCAAGCCTAGAGAGGAACAGTACAACTCCACCTACCGG |
| | | | | GTGGTGTCCGTGCTGACCGTGCTGCACCAGGATTGGCTGAACGGCA |
| | | | | AAGAGTACAAGTGCAAGGTGTCCAACAAGGCCCTGCCTGCCCCCAT |
| | | | | CGAAAAGACCATCTCCAAGGCCAAGGGCCAGCCCCGGGAACCCCAG |
| | | | | GTGTACACACTGCCCCCTAGCAGGGACGAGCTGACCAAGAACCAGG |
| | | | | TGTCCCTGACCTGTCTCGTGAAAGGCTTCTACCCCTCCGATATCGC |
| | | | | CGTGGAATGGGAGTCCAACGGCCAGCCTGAGAACAACTACAAGACC |
| | | | | ACCCCCCCTGTGCTGGACTCCGACGGCTCATTCTTCCTGTACAGCA |
| | | | | AGCTGACAGTGGACAAGTCCCGGTGGCAGCAGGGCAACGTGTTCTC |
| | | | | CTGCTCCGTGATGCACGAGGCCCTGCACAACCACTACACCCAGAAG |
| | | | | TCCCTGTCCCTGAGCCCCGGCAAGTGATGA |

| | | | |
|---|---|---|---|
| 342 | Mutated | Amino acid | EVQLVESGGGLVQPGRSLRLSCAASGFTFDDYAMHWVRQAPGKGLE |
| | 1D05 - | sequence of 1D05 | WVSGISWIRTGIGYADSVKGRFTISRDNAKNSLYLQMNSLRAEDTA |
| | HC | heavy chain with V | LYYCAKDMKGSGTYGGWFDTWGQGTLVTVSSASTKGPSVFPLAPCS |
| | mutant 2 | to A and F to S | RSTSESTAALGCLVKDYFPEPVTVSWNSGALTSGVHTFPAVLQSSG |
| | | back-mutation in | LYSLSSVVTVPSSSLGTKTYTCNVDHKPSNTKVDKRVESKYGPPCP |
| | | framework region | PCPAPELAGAPSVFLFPPKPKDTLMISRTPEVTCVVVDVSQEDPEV |
| | | to germline | QFNWYVDGVEVHNAKTKPREEQFNSTYRVVSVLTVLHQDWLNGKEY |
| | | highlighted with | KCKVSNKGLPSSIEKTISKAKGQPREPQVYTLPPSQEEMTKNQVSL |
| | | IgG1 disabled | TCLVKGFYPSDIAVEWESNGQPENNYKTTPPVLDSDGSFFLYSRLT |
| | | (LAGA) constant | VDKSRWQEGNVFSCSVMHEALHNHYTQKSLSLSLGK |
| | | region | |

TABLE S2
SEQ ID NOS: 343-538

| SEQ | | | | |
|---|---|---|---|---|
| ID | | | | |
| NO: | Name | | Description | Sequence |
| 343 | 416E01 - | | Amino acid | GFTFSNYA |
| | CDRH1 | | sequence of CDRH1 | |
| | (IMGT) | | of 416E01 using | |
| | | | IMGT | |
| 344 | 416E01 - | | Amino acid | ISFSGGTT |
| | CDRH2 | | sequence of CDRH2 | |
| | (IMGT) | | of 416E01 using | |
| | | | IMGT | |
| 345 | 416E01 - | | Amino acid | AKDEAPAGATFFDS |
| | CDRH3 | | sequence of CDRH3 | |
| | (IMGT) | | of 416E01 using | |
| | | | IMGT | |
| 346 | 416E01 - | | Amino acid | NYAMS |
| | CDRH1 | | sequence of CDRH1 | |
| | (Kabat) | | of 416E01 using | |
| | | | Kabat | |
| 347 | 416E01 - | | Amino acid | AISFSGGTTYYADSVKG |
| | CDRH2 | | sequence of CDRH2 | |
| | (Kabat) | | of 416E01 using | |
| | | | Kabat | |
| 348 | 416E01 - | | Amino acid | DEAPAGATFFDS |
| | CDRH3 | | sequence of CDRH3 | |
| | (Kabat) | | of 416E01 using | |
| | | | Kabat | |
| 349 | 416E01 - | | Amino acid | EVQLAESGGGLVQPGGSLRLSCAASGFTFSNYAMSWVRQTPGKGL |
| | Heavy | | sequence of V_{H} of | EWVSAISFSGGTTYYADSVKGRFTISRDNSKNTLYLHMNSLRADD |
| | chain | | 416E01 (mutations | TAVYYCAKDEAPAGATFFDSWGQGTLVTVSS |
| | variable | | from germline are | |
| | region | | shown in bold | |
| | | | letters) | |
| 350 | 416E01 - | | Nucleic acid | GAAGTGCAACTGGCGGAGTCTGGGGGAGGCTTGGTACAGCCGGGG |
| | Heavy | | sequence of V_{H} of | GGGTCCCTGAGACTCTCCTGTGCAGCCTCTGGATTCACCTTTAGC |
| | chain | | 416E01 | AACTATGCCATGAGTTGGGTCCGCCAGACTCCAGGAAAGGGGCTG |
| | variable | | | GAGTGGGTCTCAGCTATTAGTTTTAGTGGTGGTACTACATACTAC |
| | region | | | GCTGACTCCGTGAAGGGCCGGTTCACCATCTCCAGAGACAATTCC |
| | | | | AAGAACACGCTGTATTTGCACATGAACAGCCTGAGAGCCGATGAC |
| | | | | ACGGCCGTATATTACTGTGCGAAAGATGAGGCACCAGCTGGCGCA |
| | | | | ACCTTCTTTGACTCCTGGGGCCAGGGAACGCTGGTCACCGTCTCC |
| | | | | TCAG |
| 351 | 416E01 - | | Amino acid | EVQLAESGGGLVQPGGSLRLSCAASGFTFSNYAMSWVRQTPGKGL |
| | full | | sequence of 416E01 | EWVSAISFSGGTTYYADSVKGRFTISRDNSKNTLYLHMNSLRADD |
| | heavy | | heavy chain | TAVYYCAKDEAPAGATFFDSWGQGTLVTVSSASTKGPSVFPLAPC |
| | chain | | | SRSTSESTAALGCLVKDYFPEPVTVSWNSGALTSGVHTFPAVLQS |
| | sequence | | | SGLYSLSSVVTVPSSSLGTKTYTCNVDHKPSNTKVDKRVESKYGP |
| | | | | PCPPCPAPEFEGGPSVFLFPPKPKDTLMISRTPEVTCVVVDVSQE |
| | | | | DPEVQFNWYVDGVEVHNAKTKPREEQFNSTYRVVSVLTVLHQDWL |
| | | | | NGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSF |
| | | | | FLYSRLTVDKSRWQEGNVFSCSVMHEALHNHYTQKSLSLSLGK |
| 352 | 416E01 - | | Nucleic acid | GAAGTGCAACTGGCGGAGTCTGGGGGAGGCTTGGTACAGCCGGGG |
| | full | | sequence of 416E01 | GGGTCCCTGAGACTCTCCTGTGCAGCCTCTGGATTCACCTTTAGC |
| | heavy | | heavy chain | AACTATGCCATGAGTTGGGTCCGCCAGACTCCAGGAAAGGGGCTG |
| | chain | | | GAGTGGGTCTCAGCTATTAGTTTTAGTGGTGGTACTACATACTAC |
| | sequence | | | GCTGACTCCGTGAAGGGCCGGTTCACCATCTCCAGAGACAATTCC |
| | | | | AAGAACACGCTGTATTTGCACATGAACAGCCTGAGAGCCGATGAC |
| | | | | ACGGCCGTATATTACTGTGCGAAAGATGAGGCACCAGCTGGCGCA |
| | | | | ACCTTCTTTGACTCCTGGGGCCAGGGAACGCTGGTCACCGTCTCC |
| | | | | TCAGCCAGCACCAAGGGCCCTTCCGTGTTCCCCCTGGCCCCTTGC |
| | | | | AGCAGGAGCACCTCCGAATCCACAGCTGCCCTGGGCTGTCTGGTG |
| | | | | AAGGACTACTTTCCCGAGCCCGTGACCGTGAGCTGGAACAGCGGC |
| | | | | GCTCTGACATCCGGCGTCCACACCTTTCCTGCCGTCCTGCAGTCC |
| | | | | TCCGGCCTCTACTCCCTGTCCTCCGTGGTGACCGTGCCTAGCTCC |
| | | | | TCCCTCGGCACCAAGACCTACACCTGTAACGTGGACCACAAACCC |
| | | | | TCCAACACCAAGGTGGACAAACGGGTCGAGAGCAAGTACGGCCCT |
| | | | | CCCTGCCCTCCTTGTCCTGCCCCCGAGTTCGAAGGCGGACCCAGC |
| | | | | GTGTTCCTGTTCCCTCCTAAGCCCAAGGACACCCTCATGATCAGC |
| | | | | CGGACACCCGAGGTGACCTGCGTGGTGGTGGATGTGAGCCAGGAG |
| | | | | GACCCTGAGGTCCAGTTCAACTGGTATGTGGATGGCGTGGAGGTG |
| | | | | CACAACGCCAAGACAAAGCCCCGGGAAGAGCAGTTCAACTCCACC |
| | | | | TACAGGGTGGTCAGCGTGCTGACCGTGCTGCATCAGGACTGGCTG |
| | | | | AACGGCAAGGAGTACAAGTGCAAGGTCAGCAATAAGGGACTGCCC |
| | | | | AGCAGCATCGAGAAGACCATCTCCAAGGCTAAAGGCCAGCCCCGG |
| | | | | GAACCTCAGGTGTACACCCTGCCTCCCAGCCAGGAGGAGATGACC |
| | | | | AAGAACCAGGTGAGCCTGACCTGCCTGGTGAAGGGATTCTACCCT |
| | | | | TCCGACATCGCCGTGGAGTGGGAGTCCAACGGCCAGCCCGAGAAC |
| | | | | AATTATAAGACCACCCCTCCCGTCCTCGACAGCGACGGATCCTTC |
| | | | | TTTCTGTACTCCAGGCTGACCGTGGATAAGTCCAGGTGGCAGGAA |
| | | | | GGCAACGTGTTCAGCTGCTCCGTGATGCACGAGGCCCTGCACAAT |
| | | | | CACTACACCCAGAAGTCCCTGAGCCTGTCCCTGGGAAAG |
| 353 | 416E01 - | | Amino acid | QGIRRW |
| | CDRL1 | | sequence of CDRL1 | |
| | (IMGT) | | of 416E01 using | |
| | | | IMGT | |
| 354 | 416E01 - | | Amino acid | GAS |
| | CDRL2 | | sequence of CDRL2 | |
| | (IMGT) | | of 416E01 using | |
| | | | IMGT | |
| 355 | 416E01 - | | Amino acid | QQANSFPIT |
| | CDRL3 | | sequence of CDRL3 | |
| | (IMGT) | | of 416E01 using | |
| | | | IMGT | |
| 356 | 416E01 - | | Amino acid | RASQGIRRWLA |
| | CDRL1 | | sequence of CDRL1 | |
| | (Kabat) | | of 416E01 using | |
| | | | Kabat | |
| 357 | 416E01 - | | Amino acid | GASSLQS |
| | CDRL2 | | sequence of CDRL2 | |
| | (Kabat) | | of 416E01 using | |
| | | | Kabat | |
| 358 | 416E01 - | | Amino acid | QQANSFPIT |
| | CDRL3 | | sequence of CDRL3 | |
| | (Kabat) | | of 416E01 using | |
| | | | Kabat | |
| 359 | 416E01 - | | Amino acid | DIQMTQSPSSVSASVGDRVTITCRASQGIRRWLAWYQQKPGKAPK |
| | Light | | sequence of V_{L} of | LLISGASSLQSGVPSRFSGSGSGTDFTLIITSLQPEDFATYYCQQ |
| | chain | | 416E01 (mutations | ANSFPITFGQGTRLEIK |
| | variable | | from germline are | |
| | region | | shown in bold | |
| | | | letters) | |
| 360 | 416E01 - | | Nucleic acid | GACATCCAGATGACCCAGTCTCCATCTTCCGTGTCTGCATCTGTA |
| | Light | | sequence of V_{L} of | GGAGACAGAGTCACCATCACTTGTCGGGCGAGTCAGGGTATTAGG |
| | chain | | 416E01 | AGGTGGTTAGCCTGGTATCAGCAGAAACCAGGGAAAGCCCCTAAA |
| | variable | | | CTCCTGATCTCTGGTGCATCCAGTTTGCAAAGTGGGGTCCCATCA |
| | region | | | AGGTTCAGCGGCAGTGGATCTGGGACAGATTTCACTCTCATCATT |
| | | | | ACCAGTCTGCAGCCTGAAGATTTTGCAACTTACTATTGTCAACAG |
| | | | | GCTAACAGTTTCCCGATCACCTTCGGCCAAGGGACACGACTGGAG |
| | | | | ATCAAAC |
| 361 | 416E01 - | | Amino acid | DIQMTQSPSSVSASVGDRVTITCRASQGIRRWLAWYQQKPGKAPK |
| | full | | sequence of 416E01 | LLISGASSLQSGVPSRFSGSGSGTDFTLIITSLQPEDFATYYCQQ |
| | light | | light chain | ANSFPITFGQGTRLEIKRTVAAPSVFIFPPSDEQLKSGTASVVCL |
| | chain | | | LNNFYPREAKVQWKVDNALQSGNSQESVTEQDSKDSTYSLSSTLT |
| | sequence | | | LSKADYEKHKVYACEVTHQGLSSPVTKSFNRGEC |
| 362 | 416E01 - | | Nucleic acid | GACATCCAGATGACCCAGTCTCCATCTTCCGTGTCTGCATCTGTA |
| | full | | sequence of 416E01 | GGAGACAGAGTCACCATCACTTGTCGGGCGAGTCAGGGTATTAGG |
| | light | | light chain | AGGTGGTTAGCCTGGTATCAGCAGAAACCAGGGAAAGCCCCTAAA |
| | chain | | | CTCCTGATCTCTGGTGCATCCAGTTTGCAAAGTGGGGTCCCATCA |
| | sequence | | | AGGTTCAGCGGCAGTGGATCTGGGACAGATTTCACTCTCATCATT |
| | | | | ACCAGTCTGCAGCCTGAAGATTTTGCAACTTACTATTGTCAACAG |
| | | | | GCTAACAGTTTCCCGATCACCTTCGGCCAAGGGACACGACTGGAG |
| | | | | ATCAAACGTACGGTGGCCGCTCCCTCCGTGTTCATCTTCCCACCT |
| | | | | TCCGACGAGCAGCTGAAGTCCGGCACCGCTTCTGTCGTGTGCCTG |
| | | | | CTGAACAACTTCTACCCCCGCGAGGCCAAGGTGCAGTGGAAGGTG |
| | | | | GACAACGCCCTGCAGTCCGGCAACTCCCAGGAATCCGTGACCGAG |
| | | | | CAGGACTCCAAGGACAGCACCTACTCCCTGTCCTCCACCCTGACC |
| | | | | CTGTCCAAGGCCGACTACGAGAAGCACAAGGTGTACGCCTGCGAA |
| | | | | GTGACCCACCAGGGCCTGTCTAGCCCCGTGACCAAGTCTTTCAAC |
| | | | | CGGGGCGAGTGT |
| 363 | STIM001 - | | Amino acid | GYTFSTFG |
| | CDRH1 | | sequence of CDRH1 | |
| | | | of STIM001 using | |
| | | | IMGT | |
| 364 | STIM001 - | | Amino acid | ISAYNGDT |
| | CDRH2 | | sequence of CDRH2 | |
| | | | of STIM001 using | |
| | | | IMGT | |
| 365 | STIM001 - | | Amino acid | ARSSGHYYYYGMDV |
| | CDRH3 | | sequence of CDRH3 | |
| | | | of STIM001 using | |
| | | | IMGT | |
| 366 | STIM001 - | | Amino acid | QVQVVQSGAEVKKPGASVKVSCKASGYTFSTFGITWVRQAPGQGL |
| | Heavy | | sequence of V_{H} of | EWMGWISAYNGDTNYAQNLQGRVIMTTDTSTSTAYMELRSLRSDD |
| | chain | | STIM001 | TAVYYCARSSGHYYYYGMDVWGQGTTVTVSS |
| | variable | | | |
| | region | | | |
| 367 | STIM001 - | | Nucleic acid | CAGGTTCAGGTGGTGCAGTCTGGAGCTGAGGTGAAGAAGCCTGGG |
| | Heavy | | sequence of V_{H} of | GCCTCAGTGAAGGTCTCCTGCAAGGCTTCTGGTTACACCTTTTCC |
| | chain | | STIM001 | ACCTTTGGTATCACCTGGGTGCGACAGGCCCCTGGACAAGGGCTT |
| | variable | | | GAATGGATGGGATGGATCAGCGCTTACAATGGTGACACAAACTAT |
| | region | | | GCACAGAATCTCCAGGGCAGAGTCATCATGACCACAGACACATCC |
| | | | | ACGAGCACAGCCTACATGGAGCTGAGGAGCCTGAGATCTGACGAC |
| | | | | ACGGCCGTTTATTACTGTGCGAGGAGCAGTGGCCACTACTACTAC |
| | | | | TACGGTATGGACGTCTGGGGCCAAGGGACCACGGTCACCGTCTCC |
| | | | | TCA |
| 368 | STIM001 - | | Amino acid | QVQVVQSGAEVKKPGASVKVSCKASGYTFSTFGITWVRQAPGQGL |
| | full | | sequence of | EWMGWISAYNGDTNYAQNLQGRVIMTTDTSTSTAYMELRSLRSDD |
| | heavy | | STIM001 heavy | TAVYYCARSSGHYYYYGMDVWGQGTTVTVSSASTKGPSVFPLAPS |
| | chain | | chain | SKSTSGGTAALGCLVKDYFPEPVTVSWNSGALTSGVHTFPAVLQS |
| | sequence | | | SGLYSLSSVVTVPSSSLGTQTYICNVNHKPSNTKVDKKVEPKSCD |
| | | | | KTHTCPPCPAPELLGGPSVFLFPPKPKDTLMISRTPEVTCVVVDV |
| | | | | SHEDPEVKFNWYVDGVEVHNAKTKPREEQYNSTYRVVSVLTVLHQ |
| | | | | DWLNGKEYKCKVSNKALPAPIEKTISKAKGQPREPQVYTLPPSRD |
| | | | | ELTKNQVSLTCLVKGFYPSDIAVEWESNGQPENNYKTTPPVLDSD |
| | | | | GSFFLYSKLTVDKSRWQQGNVFSCSVMHEALHNHYTQKSLSLSPG |
| | | | | K |
| 369 | STIM001 - | | Nucleic acid | CAGGTTCAGGTGGTGCAGTCTGGAGCTGAGGTGAAGAAGCCTGGG |
| | full | | sequence of | GCCTCAGTGAAGGTCTCCTGCAAGGCTTCTGGTTACACCTTTTCC |
| | heavy | | STIM001 heavy | ACCTTTGGTATCACCTGGGTGCGACAGGCCCCTGGACAAGGGCTT |
| | chain | | chain | GAATGGATGGGATGGATCAGCGCTTACAATGGTGACACAAACTAT |
| | sequence | | | GCACAGAATCTCCAGGGCAGAGTCATCATGACCACAGACACATCC |
| | | | | ACGAGCACAGCCTACATGGAGCTGAGGAGCCTGAGATCTGACGAC |
| | | | | ACGGCCGTTTATTACTGTGCGAGGAGCAGTGGCCACTACTACTAC |
| | | | | TACGGTATGGACGTCTGGGGCCAAGGGACCACGGTCACCGTCTCC |
| | | | | TCAGCCAGCACCAAGGGCCCCTCTGTGTTCCCTCTGGCCCCTTCC |
| | | | | AGCAAGTCCACCTCTGGCGGAACAGCCGCTCTGGGCTGCCTCGTG |
| | | | | AAGGACTACTTCCCCGAGCCTGTGACCGTGTCCTGGAACTCTGGC |
| | | | | GCTCTGACCAGCGGAGTGCACACCTTCCCTGCTGTGCTGCAGTCC |
| | | | | TCCGGCCTGTACTCCCTGTCCTCCGTCGTGACCGTGCCTTCCAGC |
| | | | | TCTCTGGGCACCCAGACCTACATCTGCAACGTGAACCACAAGCCC |
| | | | | TCCAACACCAAGGTGGACAAGAAGGTGGAACCCAAGTCCTGCGAC |
| | | | | AAGACCCACACCTGTCCCCCTTGTCCTGCCCCTGAACTGCTGGGC |
| | | | | GGACCTTCCGTGTTCCTGTTCCCCCCAAAGCCCAAGGACACCCTG |
| | | | | ATGATCTCCCGGACCCCCGAAGTGACCTGCGTGGTGGTGGATGTG |
| | | | | TCCCACGAGGACCCTGAAGTGAAGTTCAATTGGTACGTGGACGGC |
| | | | | GTGGAAGTGCACAACGCCAAGACCAAGCCTAGAGAGGAACAGTAC |
| | | | | AACTCCACCTACCGGGTGGTGTCCGTGCTGACCGTGCTGCACCAG |
| | | | | GATTGGCTGAACGGCAAAGAGTACAAGTGCAAGGTGTCCAACAAG |
| | | | | GCCCTGCCTGCCCCCATCGAAAAGACCATCTCCAAGGCCAAGGGC |
| | | | | CAGCCCCGGGAACCCCAGGTGTACACACTGCCCCCTAGCAGGGAC |
| | | | | GAGCTGACCAAGAACCAGGTGTCCCTGACCTGTCTCGTGAAAGGC |
| | | | | TTCTACCCCTCCGATATCGCCGTGGAATGGGAGTCCAACGGCCAG |
| | | | | CCTGAGAACAACTACAAGACCACCCCCCCTGTGCTGGACTCCGAC |
| | | | | GGCTCATTCTTCCTGTACAGCAAGCTGACAGTGGACAAGTCCCGG |
| | | | | TGGCAGCAGGGCAACGTGTTCTCCTGCTCCGTGATGCACGAGGCC |
| | | | | CTGCACAACCACTACACCCAGAAGTCCCTGTCCCTGAGCCCCGGC |
| | | | | AAGTGATGA |
| 370 | STIM001 - | | Amino acid | QSLLHSNEYNY |
| | CDRL1 | | sequence of CDRL1 | |
| | | | of STIM001 using | |
| | | | IMGT | |
| 371 | STIM001 - | | Amino acid | LGS |
| | CDRL2 | | sequence of CDRL2 | |
| | | | of STIM001 using | |
| | | | IMGT | |
| 372 | STIM001 - | | Amino acid | MQSLQTPLT |
| | CDRL3 | | sequence of CDRL3 | |
| | | | of STIM001 using | |
| | | | IMGT | |
| 373 | STIM001 - | | Amino acid | DIVMTQSPLSLPVTPGEPASISCRSSQSLLHSNEYNYLDWYLQKP |
| | Light | | sequence of V_{L} of | GQSPQLLIFLGSNRASGVPDRFSGSGSGTDFTLKITRVEAEDVGI |
| | chain | | STIM001 | YYCMQSLQTPLTFGGGTKVEIK |
| | variable | | | |
| | region | | | |
| 374 | STIM001 - | | Nucleic acid | GATATTGTGATGACTCAGTCTCCACTCTCCCTGCCCGTCACCCCT |
| | Light | | sequence of V_{L} of | GGAGAGCCGGCCTCCATCTCCTGCAGGTCTAGTCAGAGCCTCCTG |
| | chain | | STIM001 | CATAGTAATGAATACAACTATTTGGATTGGTACCTGCAGAAGCCA |
| | variable | | | GGGCAGTCTCCACAGCTCCTGATCTTTTTGGGTTCTAATCGGGCC |
| | region | | | TCCGGGGTCCCTGACAGGTTCAGTGGCAGTGGATCAGGCACAGAT |
| | | | | TTTACACTGAAAATCACCAGAGTGGAGGCTGAGGATGTTGGAATT |
| | | | | TATTACTGCATGCAATCTCTACAAACTCCGCTCACTTTCGGCGGA |
| | | | | GGGACCAAGGTGGAGATCAAA |
| 375 | STIM001 - | | Amino acid | DIVMTQSPLSLPVTPGEPASISCRSSQSLLHSNEYNYLDWYLQKP |
| | full | | sequence of | GQSPQLLIFLGSNRASGVPDRFSGSGSGTDFTLKITRVEAEDVGI |
| | light | | STIM001 light | YYCMQSLQTPLTFGGGTKVEIK |
| | chain | | chain | RTVAAPSVFIFPPSDEQLKSGTASVVCLLNNFYPREAKVQWKVDN |
| | sequence | | | ALQSGNSQESVTEQDSKDSTYSLSSTLTLSKADYEKHKVYACEVT |
| | | | | HQGLSSPVTKSFNRGEC |
| 376 | STIM001 - | | Nucleic acid | GATATTGTGATGACTCAGTCTCCACTCTCCCTGCCCGTCACCCCT |
| | full | | sequence of | GGAGAGCCGGCCTCCATCTCCTGCAGGTCTAGTCAGAGCCTCCTG |
| | light | | STIM001 light | CATAGTAATGAATACAACTATTTGGATTGGTACCTGCAGAAGCCA |
| | chain | | chain | GGGCAGTCTCCACAGCTCCTGATCTTTTTGGGTTCTAATCGGGCC |
| | sequence | | | TCCGGGGTCCCTGACAGGTTCAGTGGCAGTGGATCAGGCACAGAT |
| | | | | TTTACACTGAAAATCACCAGAGTGGAGGCTGAGGATGTTGGAATT |
| | | | | TATTACTGCATGCAATCTCTACAAACTCCGCTCACTTTCGGCGGA |
| | | | | GGGACCAAGGTGGAGATCAAAcgtacggtggccgctccctccgtg |
| | | | | ttcatcttcccaccttccgacgagcagctgaagtccggcaccgct |
| | | | | tctgtcgtgtgcctgctgaacaacttctacccccgcgaggccaag |
| | | | | gtgcagtggaaggtggacaacgccctgcagtccggcaactcccag |
| | | | | gaatccgtgaccgagcaggactccaaggacagcacctactccctg |
| | | | | tcctccaccctgaccctgtccaaggccgactacgagaagcacaag |
| | | | | gtgtacgcctgcgaagtgacccaccagggcctgtctagccccgtg |
| | | | | accaagtctttcaaccggggcgagtgt |
| 377 | STIM002 - | | Amino acid | GYTFTSYG |
| | CDRH1 | | sequence of CDRH1 | |
| | | | of STIM002 using | |
| | | | IMGT | |
| 378 | STIM002 - | | Amino acid | ISAYNGNT |
| | CDRH2 | | sequence of CDRH2 | |
| | | | of STIM002 using | |
| | | | IMGT | |
| 379 | STIM002 - | | Amino acid | ARSTYFYGSGTLYGMDV |
| | CDRH3 | | sequence of CDRH3 | |
| | | | of STIM002 using | |
| | | | IMGT | |
| 380 | STIM002 - | | Amino acid | QVQLVQSGGEVKKPGASVKVSCKASGYTFTSYGFSWVRQAPGQGL |
| | Heavy | | sequence of V_{H} of | EWMGWISAYNGNTNYAQKLQGRVTMTTDTSTSTAYMELRSLRSDD |
| | chain | | STIM002 | TAVYYCARSTYFYGSGTLYGMDVWGQGTTVTVSS |
| | variable | | | |
| | region | | | |
| 381 | STIM002 - | | Nucleic acid | CAGGTTCAACTGGTGCAGTCTGGAGGTGAGGTGAAGAAGCCTGGG |
| | Heavy | | sequence of V_{H} of | GCCTCAGTGAAGGTCTCCTGCAAGGCTTCTGGTTACACCTTTACC |
| | chain | | STIM002 | AGCTATGGTTTCAGCTGGGTGCGACAGGCCCCTGGACAAGGACTA |
| | variable | | | GAGTGGATGGGATGGATCAGCGCTTACAATGGTAACACAAACTAT |
| | region | | | GCACAGAAGCTCCAGGGCAGAGTCACCATGACCACAGACACATCC |
| | | | | ACGAGCACAGCCTACATGGAGCTGAGGAGCTTGAGATCTGACGAC |
| | | | | ACGGCCGTGTATTACTGTGCGAGATCTACGTATTTCTATGGTTCG |
| | | | | GGGACCCTCTACGGTATGGACGTCTGGGGCCAAGGGACCACGGTC |
| | | | | ACCGTCTCCTCA |
| 382 | STIM002 - | | Amino acid | QVQLVQSGGEVKKPGASVKVSCKASGYTFTSYGFSWVRQAPGQGL |
| | full | | sequence of | EWMGWISAYNGNTNYAQKLQGRVTMTTDTSTSTAYMELRSLRSDD |
| | heavy | | STIM002 heavy | TAVYYCARSTYFYGSGTLYGMDVWGQGTTVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 383 | STIM002 - | | Nucleic acid | CAGGTTCAACTGGTGCAGTCTGGAGGTGAGGTGAAGAAGCCTGGG |
| | full | | sequence of | GCCTCAGTGAAGGTCTCCTGCAAGGCTTCTGGTTACACCTTTACC |
| | heavy | | STIM002 heavy | AGCTATGGTTTCAGCTGGGTGCGACAGGCCCCTGGACAAGGACTA |
| | chain | | chain | GAGTGGATGGGATGGATCAGCGCTTACAATGGTAACACAAACTAT |
| | sequence | | | GCACAGAAGCTCCAGGGCAGAGTCACCATGACCACAGACACATCC |
| | | | | ACGAGCACAGCCTACATGGAGCTGAGGAGCTTGAGATCTGACGAC |
| | | | | ACGGCCGTGTATTACTGTGCGAGATCTACGTATTTCTATGGTTCG |
| | | | | GGGACCCTCTACGGTATGGACGTCTGGGGCCAAGGGACCACGGTC |
| | | | | ACCGTCTCCTCA |
| | | | | GCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAG |
| | | | | GACTACTTCCCCGAGCCTGTGACCGTGTCCTGGAACTCTGGCGCT |
| | | | | CTGACCAGCGGAGTGCACACCTTCCCTGCTGTGCTGCAGTCCTCC |
| | | | | GGCCTGTACTCCCTGTCCTCCGTCGTGACCGTGCCTTCCAGCTCT |
| | | | | CTGGGCACCCAGACCTACATCTGCAACGTGAACCACAAGCCCTCC |
| | | | | AACACCAAGGTGGACAAGAAGGTGGAACCCAAGTCCTGCGACAAG |
| | | | | ACCCACACCTGTCCCCCTTGTCCTGCCCCTGAACTGCTGGGCGGA |
| | | | | CCTTCCGTGTTCCTGTTCCCCCCAAAGCCCAAGGACACCCTGATG |
| | | | | ATCTCCCGGACCCCCGAAGTGACCTGCGTGGTGGTGGATGTGTCC |
| | | | | CACGAGGACCCTGAAGTGAAGTTCAATTGGTACGTGGACGGCGTG |
| | | | | GAAGTGCACAACGCCAAGACCAAGCCTAGAGAGGAACAGTACAAC |
| | | | | TCCACCTACCGGGTGGTGTCCGTGCTGACCGTGCTGCACCAGGAT |
| | | | | TGGCTGAACGGCAAAGAGTACAAGTGCAAGGTGTCCAACAAGGCC |
| | | | | CTGCCTGCCCCCATCGAAAAGACCATCTCCAAGGCCAAGGGCCAG |
| | | | | CCCCGGGAACCCCAGGTGTACACACTGCCCCCTAGCAGGGACGAG |
| | | | | CTGACCAAGAACCAGGTGTCCCTGACCTGTCTCGTGAAAGGCTTC |
| | | | | TACCCCTCCGATATCGCCGTGGAATGGGAGTCCAACGGCCAGCCT |
| | | | | GAGAACAACTACAAGACCACCCCCCCTGTGCTGGACTCCGACGGC |
| | | | | TCATTCTTCCTGTACAGCAAGCTGACAGTGGACAAGTCCCGGTGG |
| | | | | CAGCAGGGCAACGTGTTCTCCTGCTCCGTGATGCACGAGGCCCTG |
| | | | | CACAACCACTACACCCAGAAGTCCCTGTCCCTGAGCCCCGGCAAG |
| | | | | TGATGA |
| 384 | STIM002 - | | Amino acid | QSLLHSDGYNY |
| | CDRL1 | | sequence of CDRL1 | |
| | | | of STIM002 using | |
| | | | IMGT | |
| 385 | STIM002 - | | Amino acid | LGS |
| | CDRL2 | | sequence of CDRL2 | |
| | | | of STIM002 using | |
| | | | IMGT | |
| 386 | STIM002 - | | Amino acid | MQALQTPLS |
| | CDRL3 | | sequence of CDRL3 | |
| | | | of STIM002 using | |
| | | | IMGT | |
| 387 | STIM002 - | | Amino acid | DIVMTQSPLSLPVTPGEPASISCRSSQSLLHSDGYNYLDWYLQKP |
| | Light | | sequence of V_{L} of | GQSPQLLIYLGSTRASGFPDRFSGSGSGTDFTLKISRVEAEDVGV |
| | chain | | STIM002 | YYCMQALQTPLSFGQGTKLEIK |
| | variable | | | |
| | region | | | |
| 388 | STIM002 - | | Nucleic acid | GATATTGTGATGACTCAGTCTCCACTCTCCCTGCCCGTCACCCCT |
| | Light | | sequence of V_{L} of | GGAGAGCCGGCCTCCATCTCCTGCAGGTCTAGTCAGAGCCTCCTG |
| | chain | | STIM002 | CATAGTGATGGATACAACTGTTTGGATTGGTACCTGCAGAAGCCA |
| | variable | | | GGGCAGTCTCCACAGCTCCTGATCTATTTGGGTTCTACTCGGGCC |
| | region | | | TCCGGGTTCCCTGACAGGTTCAGTGGCAGTGGATCAGGCACAGAT |
| | | | | TTTACACTGAAAATCAGCAGAGTGGAGGCTGAGGATGTTGGGGTT |
| | | | | TATTACTGCATGCAAGCTCTACAAACTCCGTGCAGTTTTGGCCAG |
| | | | | GGGACCAAGCTGGAGATCAAA |
| 389 | STIM002 - | | Amino acid | DIVMTQSPLSLPVTPGEPASISCRSSQSLLHSDGYNYLDWYLQKP |
| | full | | sequence of | GQSPQLLIYLGSTRASGFPDRFSGSGSGTDFTLKISRVEAEDVGV |
| | light | | STIM002 light | YYCMQALQTPLSFGQGTKLEIK |
| | chain | | chain | RTVAAPSVFIFPPSDEQLKSGTASVVCLLNNFYPREAKVQWKVDN |
| | sequence | | | ALQSGNSQESVTEQDSKDSTYSLSSTLTLSKADYEKHKVYACEVT |
| | | | | HQGLSSPVTKSFNRGEC |
| 390 | STIM002 - | | Nucleic acid | GATATTGTGATGACTCAGTCTCCACTCTCCCTGCCCGTCACCCCT |
| | full | | sequence of | GGAGAGCCGGCCTCCATCTCCTGCAGGTCTAGTCAGAGCCTCCTG |
| | light | | STIM002 light | CATAGTGATGGATACAACTGTTTGGATTGGTACCTGCAGAAGCCA |
| | chain | | chain | GGGCAGTCTCCACAGCTCCTGATCTATTTGGGTTCTACTCGGGCC |
| | sequence | | | TCCGGGTTCCCTGACAGGTTCAGTGGCAGTGGATCAGGCACAGAT |
| | | | | TTTACACTGAAAATCAGCAGAGTGGAGGCTGAGGATGTTGGGGTT |
| | | | | TATTACTGCATGCAAGCTCTACAAACTCCGTGCAGTTTTGGCCAG |
| | | | | GGGACCAAGCTGGAGATCAAAcgtacggtggccgctccctccgtg |
| | | | | ttcatcttcccaccttccgacgagcagctgaagtccggcaccgct |
| | | | | tctgtcgtgtgcctgctgaacaacttctacccccgcgaggccaag |
| | | | | gtgcagtggaaggtggacaacgccctgcagtccggcaactcccag |
| | | | | gaatccgtgaccgagcaggactccaaggacagcacctactccctg |
| | | | | tcctccaccctgaccctgtccaaggccgactacgagaagcacaag |
| | | | | gtgtacgcctgcgaagtgacccaccagggcctgtctagccccgtg |
| | | | | accaagtctttcaaccggggcgagtgt |
| 391 | STIM002- | | Amino acid | GYTFTSYG |
| | B - | | sequence of CDRH1 | |
| | CDRH1 | | of STIM002-B using | |
| | | | IMGT | |
| 392 | STIM002- | | Amino acid | ISAYNGNT |
| | B - | | sequence of CDRH2 | |
| | CDRH2 | | of STIM002-B using | |
| | | | IMGT | |
| 393 | STIM002- | | Amino acid | ARSTYFYGSGTLYGMDV |
| | B - | | sequence of CDRH3 | |
| | CDRH3 | | of STIM002-B using | |
| | | | IMGT | |
| 394 | STIM002- | | Amino acid | QVQLVQSGGEVKKPGASVKVSCKASGYTFTSYGFSWVRQAPGQGL |
| | B - | | sequence of V_{H} of | EWMGWISAYNGNTNYAQKLQGRVTMTTDTSTSTAYMELRSLRSDD |
| | Heavy | | STIM002-B | TAVYYCARSTYFYGSGTLYGMDVWGQGTTVTVSS |
| | chain | | | |
| | variable | | | |
| | region | | | |
| 395 | STIM002- | | Nucleic acid | CAGGTTCAACTGGTGCAGTCTGGAGGTGAGGTGAAGAAGCCTGGG |
| | B - | | sequence of V_{H} of | GCCTCAGTGAAGGTCTCCTGCAAGGCTTCTGGTTACACCTTTACC |
| | Heavy | | STIM002-B | AGCTATGGTTTCAGCTGGGTGCGACAGGCCCCTGGACAAGGACTA |
| | chain | | | GAGTGGATGGGATGGATCAGCGCTTACAATGGTAACACAAACTAT |
| | variable | | | GCACAGAAGCTCCAGGGCAGAGTCACCATGACCACAGACACATCC |
| | region | | | ACGAGCACAGCCTACATGGAGCTGAGGAGCTTGAGATCTGACGAC |
| | | | | ACGGCCGTGTATTACTGTGCGAGATCTACGTATTTCTATGGTTCG |
| | | | | GGGACCCTCTACGGTATGGACGTCTGGGGCCAAGGGACCACGGTC |
| | | | | ACCGTCTCCTCA |
| 396 | STIM002- | | Amino acid | QVQLVQSGGEVKKPGASVKVSCKASGYTFTSYGFSWVRQAPGQGL |
| | B - full | | sequence of | EWMGWISAYNGNTNYAQKLQGRVTMTTDTSTSTAYMELRSLRSDD |
| | heavy | | STIM002-B heavy | TAVYYCARSTYFYGSGTLYGMDVWGQGTTVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 397 | STIM002- | | Nucleic acid | CAGGTTCAACTGGTGCAGTCTGGAGGTGAGGTGAAGAAGCCTGGG |
| | B - full | | sequence of | GCCTCAGTGAAGGTCTCCTGCAAGGCTTCTGGTTACACCTTTACC |
| | heavy | | STIM002-B heavy | AGCTATGGTTTCAGCTGGGTGCGACAGGCCCCTGGACAAGGACTA |
| | chain | | chain | GAGTGGATGGGATGGATCAGCGCTTACAATGGTAACACAAACTAT |
| | sequence | | | GCACAGAAGCTCCAGGGCAGAGTCACCATGACCACAGACACATCC |
| | | | | ACGAGCACAGCCTACATGGAGCTGAGGAGCTTGAGATCTGACGAC |
| | | | | ACGGCCGTGTATTACTGTGCGAGATCTACGTATTTCTATGGTTCG |
| | | | | GGGACCCTCTACGGTATGGACGTCTGGGGCCAAGGGACCACGGTC |
| | | | | ACCGTCTCCTCAGCCAGCACCAAGGGCCCCTCTGTGTTCCCTCTG |
| | | | | GCCCCTTCCAGCAAGTCCACCTCTGGCGGAACAGCCGCTCTGGGC |
| | | | | TGCCTCGTGAAGGACTACTTCCCCGAGCCTGTGACCGTGTCCTGG |
| | | | | AACTCTGGCGCTCTGACCAGCGGAGTGCACACCTTCCCTGCTGTG |
| | | | | CTGCAGTCCTCCGGCCTGTACTCCCTGTCCTCCGTCGTGACCGTG |
| | | | | CCTTCCAGCTCTCTGGGCACCCAGACCTACATCTGCAACGTGAAC |
| | | | | CACAAGCCCTCCAACACCAAGGTGGACAAGAAGGTGGAACCCAAG |
| | | | | TCCTGCGACAAGACCCACACCTGTCCCCCTTGTCCTGCCCCTGAA |
| | | | | CTGCTGGGCGGACCTTCCGTGTTCCTGTTCCCCCCAAAGCCCAAG |
| | | | | GACACCCTGATGATCTCCCGGACCCCCGAAGTGACCTGCGTGGTG |
| | | | | GTGGATGTGTCCCACGAGGACCCTGAAGTGAAGTTCAATTGGTAC |
| | | | | GTGGACGGCGTGGAAGTGCACAACGCCAAGACCAAGCCTAGAGAG |
| | | | | GAACAGTACAACTCCACCTACCGGGTGGTGTCCGTGCTGACCGTG |
| | | | | CTGCACCAGGATTGGCTGAACGGCAAAGAGTACAAGTGCAAGGTG |
| | | | | TCCAACAAGGCCCTGCCTGCCCCCATCGAAAAGACCATCTCCAAG |
| | | | | GCCAAGGGCCAGCCCCGGGAACCCCAGGTGTACACACTGCCCCCT |
| | | | | AGCAGGGACGAGCTGACCAAGAACCAGGTGTCCCTGACCTGTCTC |
| | | | | GTGAAAGGCTTCTACCCCTCCGATATCGCCGTGGAATGGGAGTCC |
| | | | | AACGGCCAGCCTGAGAACAACTACAAGACCACCCCCCCTGTGCTG |
| | | | | GACTCCGACGGCTCATTCTTCCTGTACAGCAAGCTGACAGTGGAC |
| | | | | AAGTCCCGGTGGCAGCAGGGCAACGTGTTCTCCTGCTCCGTGATG |
| | | | | CACGAGGCCCTGCACAACCACTACACCCAGAAGTCCCTGTCCCTG |
| | | | | AGCCCCGGCAAGTGATGA |
| 398 | STIM002- | | Amino acid | QSLLHSDGYNC |
| | B - | | sequence of CDRL1 | |
| | CDRL1 | | of STIM002-B using | |
| | | | IMGT | |
| 399 | STIM002- | | Amino acid | LGS |
| | B - | | sequence of CDRL2 | |
| | CDRL2 | | of STIM002-B using | |
| | | | IMGT | |
| 400 | STIM002- | | Amino acid | MQALQTPCS |
| | B - | | sequence of CDRL3 | |
| | CDRL3 | | of STIM002-B using | |
| | | | IMGT | |
| 401 | STIM002- | | Amino acid | DIVMTQSPLSLPVTPGEPASISCRSSQSLLHSDGYNCLDWYLQKP |
| | B - | | sequence of V_{L} of | GQSPQLLIYLGSTRASGFPDRFSGSGSGTDFTLKISRVEAEDVGV |
| | Light | | STIM002-B | YYCMQALQTPCSFGQGTKLEIK |
| | chain | | | |
| | variable | | | |
| | region | | | |
| 402 | STIM002- | | Nucleic acid | GATATTGTGATGACTCAGTCTCCACTCTCCCTGCCCGTCACCCCT |
| | B - | | sequence of V_{L} of | GGAGAGCCGGCCTCCATCTCCTGCAGGTCTAGTCAGAGCCTCCTG |
| | Light | | STIM002-B | CATAGTGATGGATACAACTGTTTGGATTGGTACCTGCAGAAGCCA |
| | chain | | | GGGCAGTCTCCACAGCTCCTGATCTATTTGGGTTCTACTCGGGCC |
| | variable | | | TCCGGGTTCCCTGACAGGTTCAGTGGCAGTGGATCAGGCACAGAT |
| | region | | | TTTACACTGAAAATCAGCAGAGTGGAGGCTGAGGATGTTGGGGTT |
| | | | | TATTACTGCATGCAAGCTCTACAAACTCCGTGCAGTTTTGGCCAG |
| | | | | GGGACCAAGCTGGAGATCAAA |
| 403 | STIM002- | | Amino acid | DIVMTQSPLSLPVTPGEPASISCRSSQSLLHSDGYNCLDWYLQKP |
| | B - full | | sequence of | GQSPQLLIYLGSTRASGFPDRFSGSGSGTDFTLKISRVEAEDVGV |
| | light | | STIM002-B light | YYCMQALQTPCSFGQGTKLEIK |
| | chain | | chain | RTVAAPSVFIFPPSDEQLKSGTASVVCLLNNFYPREAKVQWKVDN |
| | sequence | | | ALQSGNSQESVTEQDSKDSTYSLSSTLTLSKADYEKHKVYACEVT |
| | | | | HQGLSSPVTKSFNRGEC |
| 404 | STIM002- | | Nucleic acid | GATATTGTGATGACTCAGTCTCCACTCTCCCTGCCCGTCACCCCT |
| | B - full | | sequence of | GGAGAGCCGGCCTCCATCTCCTGCAGGTCTAGTCAGAGCCTCCTG |
| | light | | STIM002-B light | CATAGTGATGGATACAACTGTTTGGATTGGTACCTGCAGAAGCCA |
| | chain | | chain | GGGCAGTCTCCACAGCTCCTGATCTATTTGGGTTCTACTCGGGCC |
| | sequence | | | TCCGGGTTCCCTGACAGGTTCAGTGGCAGTGGATCAGGCACAGAT |
| | | | | TTTACACTGAAAATCAGCAGAGTGGAGGCTGAGGATGTTGGGGTT |
| | | | | TATTACTGCATGCAAGCTCTACAAACTCCGTGCAGTTTTGGCCAG |
| | | | | GGGACCAAGCTGGAGATCAAAcgtacggtggccgctccctccgtg |
| | | | | ttcatcttcccaccttccgacgagcagctgaagtccggcaccgct |
| | | | | tctgtcgtgtgcctgctgaacaacttctacccccgcgaggccaag |
| | | | | gtgcagtggaaggtggacaacgccctgcagtccggcaactcccag |
| | | | | gaatccgtgaccgagcaggactccaaggacagcacctactccctg |
| | | | | tcctccaccctgaccctgtccaaggccgactacgagaagcacaag |
| | | | | gtgtacgcctgcgaagtgacccaccagggcctgtctagccccgtg |
| | | | | accaagtctttcaaccggggcgagtgt |
| 405 | STIM003 - | | Amino acid | GVTFDDYG |
| | CDRH1 | | sequence of CDRH1 | |
| | | | of STIM003 using | |
| | | | IMGT | |
| 406 | STIM003 - | | Amino acid | INWNGGDT |
| | CDRH2 | | sequence of CDRH2 | |
| | | | of STIM003 using | |
| | | | IMGT | |
| 407 | STIM003 - | | Amino acid | ARDFYGSGSYYHVPFDY |
| | CDRH3 | | sequence of CDRH3 | |
| | | | of STIM003 using | |
| | | | IMGT | |
| 408 | STIM003 - | | Amino acid | EVQLVESGGGVVRPGGSLRLSCVASGVTFDDYGMSWVRQAPGKGL |
| | Heavy | | sequence of V_{H} of | EWVSGINWNGGDTDYSDSVKGRFTISRDNAKNSLYLQMNSLRAED |
| | chain | | STIM003 | TALYYCARDFYGSGSYYHVPFDYWGQGILVTVSS |
| | variable | | | |
| | region | | | |
| 409 | STIM003 - | | Nucleic acid | GAGGTGCAGCTGGTGGAGTCTGGGGGAGGTGTGGTACGGCCTGGG |
| | Heavy | | sequence of V_{H} of | GGGTCCCTGAGACTCTCCTGTGTAGCCTCTGGAGTCACCTTTGAT |
| | chain | | STIM003 | GATTATGGCATGAGCTGGGTCCGCCAAGCTCCAGGGAAGGGGCTG |
| | variable | | | GARTGGGTCTCTGGTATTAATTGGAATGGTGGCGACACAGATTAT |
| | region | | | TCAGACTCTGTGAAGGGCCGATTCACCATCTCCAGAGACAACGCC |
| | | | | AAGAACTCCCTGTATCTACAAATGAATAGTCTGAGAGCCGAGGAC |
| | | | | ACGGCCTTGTATTACTGTGCGAGGGATTTCTATGGTTCGGGGAGT |
| | | | | TATTATCACGTTCCTTTTGACTACTGGGGCCAGGGAATCCTGGTC |
| | | | | ACCGTCTCCTCA |
| 410 | STIM003 - | | Amino acid | EVQLVESGGGVVRPGGSLRLSCVASGVTFDDYGMSWVRQAPGKGL |
| | full | | sequence of | EWVSGINWNGGDTDYSDSVKGRFTISRDNAKNSLYLQMNSLRAED |
| | heavy | | STIM003 heavy | TALYYCARDFYGSGSYYHVPFDYWGQGILVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 411 | STIM003 - | | Nucleic acid | GAGGTGCAGCTGGTGGAGTCTGGGGGAGGTGTGGTACGGCCTGGG |
| | full | | sequence of | GGGTCCCTGAGACTCTCCTGTGTAGCCTCTGGAGTCACCTTTGAT |
| | heavy | | STIM003 heavy | GATTATGGCATGAGCTGGGTCCGCCAAGCTCCAGGGAAGGGGCTG |
| | chain | | chain | GARTGGGTCTCTGGTATTAATTGGAATGGTGGCGACACAGATTAT |
| | sequence | | | TCAGACTCTGTGAAGGGCCGATTCACCATCTCCAGAGACAACGCC |
| | | | | AAGAACTCCCTGTATCTACAAATGAATAGTCTGAGAGCCGAGGAC |
| | | | | ACGGCCTTGTATTACTGTGCGAGGGATTTCTATGGTTCGGGGAGT |
| | | | | TATTATCACGTTCCTTTTGACTACTGGGGCCAGGGAATCCTGGTC |
| | | | | ACCGTCTCCTCAGCCAGCACCAAGGGCCCCTCTGTGTTCCCTCTG |
| | | | | GCCCCTTCCAGCAAGTCCACCTCTGGCGGAACAGCCGCTCTGGGC |
| | | | | TGCCTCGTGAAGGACTACTTCCCCGAGCCTGTGACCGTGTCCTGG |
| | | | | AACTCTGGCGCTCTGACCAGCGGAGTGCACACCTTCCCTGCTGTG |
| | | | | CTGCAGTCCTCCGGCCTGTACTCCCTGTCCTCCGTCGTGACCGTG |
| | | | | CCTTCCAGCTCTCTGGGCACCCAGACCTACATCTGCAACGTGAAC |
| | | | | CACAAGCCCTCCAACACCAAGGTGGACAAGAAGGTGGAACCCAAG |
| | | | | TCCTGCGACAAGACCCACACCTGTCCCCCTTGTCCTGCCCCTGAA |
| | | | | CTGCTGGGCGGACCTTCCGTGTTCCTGTTCCCCCCAAAGCCCAAG |
| | | | | GACACCCTGATGATCTCCCGGACCCCCGAAGTGACCTGCGTGGTG |
| | | | | GTGGATGTGTCCCACGAGGACCCTGAAGTGAAGTTCAATTGGTAC |
| | | | | GTGGACGGCGTGGAAGTGCACAACGCCAAGACCAAGCCTAGAGAG |
| | | | | GAACAGTACAACTCCACCTACCGGGTGGTGTCCGTGCTGACCGTG |
| | | | | CTGCACCAGGATTGGCTGAACGGCAAAGAGTACAAGTGCAAGGTG |
| | | | | TCCAACAAGGCCCTGCCTGCCCCCATCGAAAAGACCATCTCCAAG |
| | | | | GCCAAGGGCCAGCCCCGGGAACCCCAGGTGTACACACTGCCCCCT |
| | | | | AGCAGGGACGAGCTGACCAAGAACCAGGTGTCCCTGACCTGTCTC |
| | | | | GTGAAAGGCTTCTACCCCTCCGATATCGCCGTGGAATGGGAGTCC |
| | | | | AACGGCCAGCCTGAGAACAACTACAAGACCACCCCCCCTGTGCTG |
| | | | | GACTCCGACGGCTCATTCTTCCTGTACAGCAAGCTGACAGTGGAC |
| | | | | AAGTCCCGGTGGCAGCAGGGCAACGTGTTCTCCTGCTCCGTGATG |
| | | | | CACGAGGCCCTGCACAACCACTACACCCAGAAGTCCCTGTCCCTG |
| | | | | AGCCCCGGCAAGTGATGA |
| 412 | STIM003 - | | Amino acid | QSVSRSY |
| | CDRL1 | | sequence of CDRL1 | |
| | | | of STIM003 using | |
| | | | IMGT | |
| 413 | STIM003 - | | Amino acid | GAS |
| | CDRL2 | | sequence of CDRL2 | |
| | | | of STIM003 using | |
| | | | IMGT | |
| 414 | STIM003 - | | Amino acid | HQYDMSPFT |
| | CDRL3 | | sequence of CDRL3 | |
| | | | of STIM003 using | |
| | | | IMGT | |
| 415 | STIM003 - | | Amino acid | EIVLTQSPGTLSLSPGERATLSCRASQSVSRSYLAWYQQKRGQAP |
| | Light | | sequence of V_{L} of | RLLIYGASSRATGIPDRFSGDGSGTDFTLSISRLEPEDFAVYYCH |
| | chain | | STIM003 | QYDMSPFTFGPGTKVDIK |
| | variable | | | |
| | region | | | |
| 416 | STIM003 - | | Nucleic acid | GAAATTGTGTTGACGCAGTCTCCAGGGACCCTGTCTTTGTCTCCA |
| | Light | | sequence of V_{L} of | GGGGAAAGAGCCACCCTCTCCTGCAGGGCCAGTCAGAGTGTTAGC |
| | chain | | STIM003 | AGAAGCTACTTAGCCTGGTACCAGCAGAAACGTGGCCAGGCTCCC |
| | variable | | | AGGCTCCTCATCTATGGTGCATCCAGCAGGGCCACTGGCATCCCA |
| | region | | | GACAGGTTCAGTGGCGATGGGTCTGGGACAGACTTCACTCTCTCC |
| | | | | ATCAGCAGACTGGAGCCTGAAGATTTTGCAGTGTATTACTGTCAC |
| | | | | CAGTATGATATGTCACCATTCACTTTCGGCCCTGGGACCAAAGTG |
| | | | | GATATCAAA |
| 417 | STIM003 - | | Amino acid | EIVLTQSPGTLSLSPGERATLSCRASQSVSRSYLAWYQQKRGQAP |
| | full | | sequence of | RLLIYGASSRATGIPDRFSGDGSGTDFTLSISRLEPEDFAVYYCH |
| | light | | STIM003 light | QYDMSPFTFGPGTKVDIKRTVAAPSVFIFPPSDEQLKSGTASVVC |
| | chain | | chain | LLNNFYPREAKVQWKVDNALQSGNSQESVTEQDSKDSTYSLSSTL |
| | sequence | | | TLSKADYEKHKVYACEVTHQGLSSPVTKSFNRGEC |
| 418 | STIM003 - | | Nucleic acid | GAAATTGTGTTGACGCAGTCTCCAGGGACCCTGTCTTTGTCTCCA |
| | full | | sequence of | GGGGAAAGAGCCACCCTCTCCTGCAGGGCCAGTCAGAGTGTTAGC |
| | light | | STIM003 light | AGAAGCTACTTAGCCTGGTACCAGCAGAAACGTGGCCAGGCTCCC |
| | chain | | chain | AGGCTCCTCATCTATGGTGCATCCAGCAGGGCCACTGGCATCCCA |
| | sequence | | | GACAGGTTCAGTGGCGATGGGTCTGGGACAGACTTCACTCTCTCC |
| | | | | ATCAGCAGACTGGAGCCTGAAGATTTTGCAGTGTATTACTGTCAC |
| | | | | CAGTATGATATGTCACCATTCACTTTCGGCCCTGGGACCAAAGTG |
| | | | | GATATCAAAcgtacggtggccgctccctccgtgttcatcttccca |
| | | | | ccttccgacgagcagctgaagtccggcaccgcttctgtcgtgtgc |
| | | | | ctgctgaacaacttctacccccgcgaggccaaggtgcagtggaag |
| | | | | gtggacaacgccctgcagtccggcaactcccaggaatccgtgacc |
| | | | | gagcaggactccaaggacagcacctactccctgtcctccaccctg |
| | | | | accctgtccaaggccgactacgagaagcacaaggtgtacgcctgc |
| | | | | gaagtgacccaccagggcctgtctagccccgtgaccaagtctttc |
| | | | | aaccggggcgagtgt |
| 419 | STIM004 - | | Amino acid | GLTFDDYG |
| | CDRH1 | | sequence of CDRH1 | |
| | | | of STIM004 using | |
| | | | IMGT | |
| 420 | STIM004 - | | Amino acid | INWNGDNT |
| | CDRH2 | | sequence of CDRH2 | |
| | | | of STIM004 using | |
| | | | IMGT | |
| 421 | STIM004 - | | Amino acid | ARDYYGSGSYYNVPFDY |
| | CDRH3 | | sequence of CDRH3 | |
| | | | of STIM004 using | |
| | | | IMGT | |
| 422 | STIM004 - | | Amino acid | EVQLVESGGGVVRPGGSLRLSCAASGLTFDDYGMSWVRQVPGKGL |
| | Heavy | | sequence of V_{H} of | EWVSGINWNGDNTDYADSVKGRFTISRDNAKNSLYLQMNSLRAED |
| | chain | | STIM004 | TALYYCARDYYGSGSYYNVPFDYWGQGTLVTVSS |
| | variable | | | |
| | region | | | |
| 423 | STIM004 - | | Nucleic acid | GAGGTGCAGCTGGTGGAGTCTGGGGGAGGTGTGGTACGGCCTGGG |
| | Heavy | | sequence of V_{H} of | GGGTCCCTGAGACTCTCCTGTGCAGCCTCTGGACTCACCTTTGAT |
| | chain | | STIM004 | GATTATGGCATGAGCTGGGTCCGCCAAGTTCCAGGGAAGGGGCTG |
| | variable | | | GAGTGGGTCTCTGGTATTAATTGGAATGGTGATAACACAGATTAT |
| | region | | | GCAGACTCTGTGAAGGGCCGATTCACCATCTCCAGAGACAACGCC |
| | | | | AAGAACTCCCTGTATCTGCAAATGAACAGTCTGAGAGCCGAGGAC |
| | | | | ACGGCCTTGTATTACTGTGCGAGGGATTACTATGGTTCGGGGAGT |
| | | | | TATTATAACGTTCCTTTTGACTACTGGGGCCAGGGAACCCTGGTC |
| | | | | ACCGTCTCCTCA |
| 424 | STIM004 - | | Amino acid | EVQLVESGGGVVRPGGSLRLSCAASGLTFDDYGMSWVRQVPGKGL |
| | full | | sequence of | EWVSGINWNGDNTDYADSVKGRFTISRDNAKNSLYLQMNSLRAED |
| | heavy | | STIM004 heavy | TALYYCARDYYGSGSYYNVPFDYWGQGTLVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 425 | STIM004 - | | Nucleic acid | GAGGTGCAGCTGGTGGAGTCTGGGGGAGGTGTGGTACGGCCTGGG |
| | full | | sequence of | GGGTCCCTGAGACTCTCCTGTGTAGCCTCTGGAGTCACCTTTGAT |
| | heavy | | STIM004 heavy | GATTATGGCATGAGCTGGGTCCGCCAAGTTCCAGGGAAGGGGCTG |
| | chain | | chain | GAGTGGGTCTCTGGTATTAATTGGAATGGTGATAACACAGATTAT |
| | sequence | | | GCAGACTCTGTGAAGGGCCGATTCACCATCTCCAGAGACAACGCC |
| | | | | AAGAACTCCCTGTATCTGCAAATGAACAGTCTGAGAGCCGAGGAC |
| | | | | ACGGCCTTGTATTACTGTGCGAGGGATTACTATGGTTCGGGGAGT |
| | | | | TATTATAACGTTCCTTTTGACTACTGGGGCCAGGGAACCCTGGTC |
| | | | | ACCGTCTCCTCAGCCAGCACCAAGGGCCCCTCTGTGTTCCCTCTG |
| | | | | GCCCCTTCCAGCAAGTCCACCTCTGGCGGAACAGCCGCTCTGGGC |
| | | | | TGCCTCGTGAAGGACTACTTCCCCGAGCCTGTGACCGTGTCCTGG |
| | | | | AACTCTGGCGCTCTGACCAGCGGAGTGCACACCTTCCCTGCTGTG |
| | | | | CTGCAGTCCTCCGGCCTGTACTCCCTGTCCTCCGTCGTGACCGTG |
| | | | | CCTTCCAGCTCTCTGGGCACCCAGACCTACATCTGCAACGTGAAC |
| | | | | CACAAGCCCTCCAACACCAAGGTGGACAAGAAGGTGGAACCCAAG |
| | | | | TCCTGCGACAAGACCCACACCTGTCCCCCTTGTCCTGCCCCTGAA |
| | | | | CTGCTGGGCGGACCTTCCGTGTTCCTGTTCCCCCCAAAGCCCAAG |
| | | | | GACACCCTGATGATCTCCCGGACCCCCGAAGTGACCTGCGTGGTG |
| | | | | GTGGATGTGTCCCACGAGGACCCTGAAGTGAAGTTCAATTGGTAC |
| | | | | GTGGACGGCGTGGAAGTGCACAACGCCAAGACCAAGCCTAGAGAG |
| | | | | GAACAGTACAACTCCACCTACCGGGTGGTGTCCGTGCTGACCGTG |
| | | | | CTGCACCAGGATTGGCTGAACGGCAAAGAGTACAAGTGCAAGGTG |
| | | | | TCCAACAAGGCCCTGCCTGCCCCCATCGAAAAGACCATCTCCAAG |
| | | | | GCCAAGGGCCAGCCCCGGGAACCCCAGGTGTACACACTGCCCCCT |
| | | | | AGCAGGGACGAGCTGACCAAGAACCAGGTGTCCCTGACCTGTCTC |
| | | | | GTGAAAGGCTTCTACCCCTCCGATATCGCCGTGGAATGGGAGTCC |
| | | | | AACGGCCAGCCTGAGAACAACTACAAGACCACCCCCCCTGTGCTG |
| | | | | GACTCCGACGGCTCATTCTTCCTGTACAGCAAGCTGACAGTGGAC |
| | | | | AAGTCCCGGTGGCAGCAGGGCAACGTGTTCTCCTGCTCCGTGATG |
| | | | | CACGAGGCCCTGCACAACCACTACACCCAGAAGTCCCTGTCCCTG |
| | | | | AGCCCCGGCAAGTGATGA |
| 426 | STIM004 - | | Amino acid | QSVSSSY |
| | CDRL1 | | sequence of CDRL1 | |
| | | | of STIM004 using | |
| | | | IMGT | |
| 427 | STIM004 - | | Amino acid | GAS |
| | CDRL2 | | sequence of CDRL2 | |
| | | | of STIM004 using | |
| | | | IMGT | |
| 428 | STIM004 - | | Amino acid | QQYGSSPF |
| | CDRL3 | | sequence of CDRL3 | |
| | | | of STIM004 using | |
| | | | IMGT | |
| 429 | STIM004 | | Amino acid | EIVLTQSPGTLSLSPGERATLSCRASQSVSSSYLAWYQQKPGQAP |
| | Corrected | | sequence of | RLLIYGASSRATGIPDRFSGSGSGTDFTLTIRRLEPEDFAVYYCQ |
| | light | | corrected V_{L} of | QYGSSPFFGPGTKVDIK |
| | chain | | STIM004 | |
| | variable | | | |
| | region | | | |
| 430 | STIM004 | | Nucleic acid | GAAATTGTGTTGACGCAGTCTCCAGGCACCCTGTCTTTGTCTCCA |
| | Corrected | | sequence of | GGGGAAAGAGCCACCCTCTCCTGCAGGGCCAGTCAGAGTGTTAGC |
| | light | | corrected V_{L} of | AGCAGCTACTTAGCCTGGTACCAGCAGAAACCTGGCCAGGCTCCC |
| | chain | | STIM004 | AGGCTCCTCATATATGGTGCATCCAGCAGGGCCACTGGCATCCCA |
| | variable | | | GACAGGTTCAGTGGCAGTGGGTCTGGGACAGACTTCACTCTCACC |
| | region | | | ATCAGAAGACTGGAGCCTGAAGATTTTGCAGTGTATTACTGTCAG |
| | | | | CAGTATGGTAGTTCACCATTCTTCGGCCCTGGGACCAAAGTGGAT |
| | | | | ATCAAA |
| 431 | STIM004 - | | Nucleic acid | GAAATTGTGTTGACGCAGTCTCCAGGCACCCTGTCTTTGTCTCCA |
| | Light | | sequence of V_{L} of | GGGGAAAGAGCCACCCTCTCCTGCAGGGCCAGTCAGAGTGTTAGC |
| | chain | | STIM004 | AGCAGCTACTTAGCCTGGTACCAGCAGAAACCTGGCCAGGCTCCC |
| | variable | | | AGGCTCCTCATATATGGTGCATCCAGCAGGGCCACTGGCATCCCA |
| | region | | | GACAGGTTCAGTGGCAGTGGGTCTGGGACAGACTTCACTCTCACC |
| | | | | ATCAGAAGACTGGAGCCTGAAGATTTTGCAGTGTATTACTGTCAG |
| | | | | CAGTATGGTAGTTCACCATTCACTTCGGCCCTGGGACCAAAGTGG |
| | | | | ATATCAAA |
| 432 | STIM004 - | | Amino acid | EIVLTQSPGTLSLSPGERATLSCRASQSVSSSYLAWYQQKPGQAP |
| | full | | sequence of | RLLIYGASSRATGIPDRFSGSGSGTDFTLTIRRLEPEDFAVYYCQ |
| | corrected | | STIM004 light | QYGSSPFFGPGTKVDIKRTVAAPSVFIFPPSDEQLKSGTASVVCL |
| | light | | chain | LNNFYPREAKVQWKVDNALQSGNSQESVTEQDSKDSTYSLSSTLT |
| | chain | | | LSKADYEKHKVYACEVTHQGLSSPVTKSFNRGEC |
| | sequence | | | |
| 433 | STIM004 - | | Nucleic acid | GAAATTGTGTTGACGCAGTCTCCAGGCACCCTGTCTTTGTCTCCA |
| | full | | sequence of | GGGGAAAGAGCCACCCTCTCCTGCAGGGCCAGTCAGAGTGTTAGC |
| | corrected | | corrected STIM004 | AGCAGCTACTTAGCCTGGTACCAGCAGAAACCTGGCCAGGCTCCC |
| | light | | light chain | AGGCTCCTCATATATGGTGCATCCAGCAGGGCCACTGGCATCCCA |
| | chain | | | GACAGGTTCAGTGGCAGTGGGTCTGGGACAGACTTCACTCTCACC |
| | sequence | | | ATCAGAAGACTGGAGCCTGAAGATTTTGCAGTGTATTACTGTCAG |
| | | | | CAGTATGGTAGTTCACCATTCTTCGGCCCTGGGACCAAAGTGGAT |
| | | | | ATCAAAcgtacggtggccgctccctccgtgttcatcttcccacct |
| | | | | tccgacgagcagctgaagtccggcaccgcttctgtcgtgtgcctg |
| | | | | ctgaacaacttctacccccgcgaggccaaggtgcagtggaaggtg |
| | | | | gacaacgccctgcagtccggcaactcccaggaatccgtgaccgag |
| | | | | caggactccaaggacagcacctactccctgtcctccaccctgacc |
| | | | | ctgtccaaggccgactacgagaagcacaaggtgtacgcctgcgaa |
| | | | | gtgacccaccagggcctgtctagccccgtgaccaagtctttcaac |
| | | | | cggggcgagtgt |
| 434 | STIM004 - | | Nucleic acid | GAAATTGTGTTGACGCAGTCTCCAGGCACCCTGTCTTTGTCTCCA |
| | full | | sequence of | GGGGAAAGAGCCACCCTCTCCTGCAGGGCCAGTCAGAGTGTTAGC |
| | light | | STIM004 light | AGCAGCTACTTAGCCTGGTACCAGCAGAAACCTGGCCAGGCTCCC |
| | chain | | chain | AGGCTCCTCATATATGGTGCATCCAGCAGGGCCACTGGCATCCCA |
| | sequence | | | GACAGGTTCAGTGGCAGTGGGTCTGGGACAGACTTCACTCTCACC |
| | | | | ATCAGAAGACTGGAGCCTGAAGATTTTGCAGTGTATTACTGTCAG |
| | | | | CAGTATGGTAGTTCACCATTCACTTCGGCCCTGGGACCAAAGTGG |
| | | | | ATATCAAAcgtacggtggccgctccctccgtgttcatcttcccac |
| | | | | cttccgacgagcagctgaagtccggcaccgcttctgtcgtgtgcc |
| | | | | tgctgaacaacttctacccccgcgaggccaaggtgcagtggaagg |
| | | | | tggacaacgccctgcagtccggcaactcccaggaatccgtgaccg |
| | | | | agcaggactccaaggacagcacctactccctgtcctccaccctga |
| | | | | ccctgtccaaggccgactacgagaagcacaaggtgtacgcctgcg |
| | | | | aagtgacccaccagggcctgtctagccccgtgaccaagtctttca |
| | | | | accggggcgagtgt |
| 435 | STIM005 - | | Amino acid | GYTFNSYG |
| | CDRH1 | | sequence of CDRH1 | |
| | | | of STIM005 using | |
| | | | IMGT | |
| 436 | STIM005 - | | Amino acid | ISVHNGNT |
| | CDRH2 | | sequence of CDRH2 | |
| | | | of STIM005 using | |
| | | | IMGT | |
| 437 | STIM005 - | | Amino acid | ARAGYDILTDFSDAFDI |
| | CDRH3 | | sequence of CDRH3 | |
| | | | of STIM005 using | |
| | | | IMGT | |
| 438 | STIM005 - | | Amino acid | QVQLVQSGAEVKKPGASVKVSCKASGYTFNSYGIIWVRQAPGQGL |
| | Heavy | | sequence of V_{H} of | EWMGWISVHNGNTNCAQKLQGRVTMTTDTSTSTAYMELRSLRTDD |
| | chain | | STIM005 | TAVYYCARAGYDILTDFSDAFDIWGHGTMVTVSS |
| | variable | | | |
| | region | | | |
| 439 | STIM005 - | | Nucleic acid | CAGGTTCAGTTGGTGCAGTCTGGAGCTGAGGTGAAGAAGCCTGGG |
| | Heavy | | sequence of V_{H} of | GCCTCAGTGAAGGTCTCCTGCAAGGCTTCTGGTTACACCTTTAAT |
| | chain | | STIM005 | AGTTATGGTATCATCTGGGTGCGACAGGCCCCTGGACAAGGGCTT |
| | variable | | | GAGTGGATGGGATGGATCAGCGTTCACAATGGTAACACAAACTGT |
| | region | | | GCACAGAAGCTCCAGGGTAGAGTCACCATGACCACAGACACATCC |
| | | | | ACGAGCACAGCCTACATGGAGCTGAGGAGCCTGAGAACTGACGAC |
| | | | | ACGGCCGTGTATTACTGTGCGAGAGCGGGTTACGATATTTTGACT |
| | | | | GATTTTTCCGATGCTTTTGATATCTGGGGCCACGGGACAATGGTC |
| | | | | ACCGTCTCTTCA |
| 440 | STIM005 - | | Amino acid | QVQLVQSGAEVKKPGASVKVSCKASGYTFNSYGIIWVRQAPGQGL |
| | full | | sequence of | EWMGWISVHNGNTNCAQKLQGRVTMTTDTSTSTAYMELRSLRTDD |
| | heavy | | STIM005 heavy | TAVYYCARAGYDILTDFSDAFDIWGHGTMVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 441 | STIM005 - | | Nucleic acid | CAGGTTCAGTTGGTGCAGTCTGGAGCTGAGGTGAAGAAGCCTGGG |
| | full | | sequence of | GCCTCAGTGAAGGTCTCCTGCAAGGCTTCTGGTTACACCTTTAAT |
| | heavy | | STIM005 heavy | AGTTATGGTATCATCTGGGTGCGACAGGCCCCTGGACAAGGGCTT |
| | chain | | chain | GAGTGGATGGGATGGATCAGCGTTCACAATGGTAACACAAACTGT |
| | sequence | | | GCACAGAAGCTCCAGGGTAGAGTCACCATGACCACAGACACATCC |
| | | | | ACGAGCACAGCCTACATGGAGCTGAGGAGCCTGAGAACTGACGAC |
| | | | | ACGGCCGTGTATTACTGTGCGAGAGCGGGTTACGATATTTTGACT |
| | | | | GATTTTTCCGATGCTTTTGATATCTGGGGCCACGGGACAATGGTC |
| | | | | ACCGTCTCTTCA |
| | | | | GCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAG |
| | | | | GACTACTTCCCCGAGCCTGTGACCGTGTCCTGGAACTCTGGCGCT |
| | | | | CTGACCAGCGGAGTGCACACCTTCCCTGCTGTGCTGCAGTCCTCC |
| | | | | GGCCTGTACTCCCTGTCCTCCGTCGTGACCGTGCCTTCCAGCTCT |
| | | | | CTGGGCACCCAGACCTACATCTGCAACGTGAACCACAAGCCCTCC |
| | | | | AACACCAAGGTGGACAAGAAGGTGGAACCCAAGTCCTGCGACAAG |
| | | | | ACCCACACCTGTCCCCCTTGTCCTGCCCCTGAACTGCTGGGCGGA |
| | | | | CCTTCCGTGTTCCTGTTCCCCCCAAAGCCCAAGGACACCCTGATG |
| | | | | ATCTCCCGGACCCCCGAAGTGACCTGCGTGGTGGTGGATGTGTCC |
| | | | | CACGAGGACCCTGAAGTGAAGTTCAATTGGTACGTGGACGGCGTG |
| | | | | GAAGTGCACAACGCCAAGACCAAGCCTAGAGAGGAACAGTACAAC |
| | | | | TCCACCTACCGGGTGGTGTCCGTGCTGACCGTGCTGCACCAGGAT |
| | | | | TGGCTGAACGGCAAAGAGTACAAGTGCAAGGTGTCCAACAAGGCC |
| | | | | CTGCCTGCCCCCATCGAAAAGACCATCTCCAAGGCCAAGGGCCAG |
| | | | | CCCCGGGAACCCCAGGTGTACACACTGCCCCCTAGCAGGGACGAG |
| | | | | CTGACCAAGAACCAGGTGTCCCTGACCTGTCTCGTGAAAGGCTTC |
| | | | | TACCCCTCCGATATCGCCGTGGAATGGGAGTCCAACGGCCAGCCT |
| | | | | GAGAACAACTACAAGACCACCCCCCCTGTGCTGGACTCCGACGGC |
| | | | | TCATTCTTCCTGTACAGCAAGCTGACAGTGGACAAGTCCCGGTGG |
| | | | | CAGCAGGGCAACGTGTTCTCCTGCTCCGTGATGCACGAGGCCCTG |
| | | | | CACAACCACTACACCCAGAAGTCCCTGTCCCTGAGCCCCGGCAAG |
| | | | | TGATGA |
| 442 | STIM005 - | | Amino acid | QNINNF |
| | CDRL1 | | sequence of CDRL1 | |
| | | | of STIM005 using | |
| | | | IMGT | |
| 443 | STIM005 - | | Amino acid | AAS |
| | CDRL2 | | sequence of CDRL2 | |
| | | | of STIM005 using | |
| | | | IMGT | |
| 444 | STIM005 - | | Amino acid | QQSYGIPW |
| | CDRL3 | | sequence of CDRL3 | |
| | | | of STIM005 using | |
| | | | IMGT | |
| 445 | STIM005 - | | Amino acid | DIQMTQSPSSLSASVGDRVTITCRASQNINNFLNWYQQKEGKGPK |
| | Light | | sequence of V_{L} of | LLIYAASSLQRGIPSTFSGSGSGTDFTLTISSLQPEDFATYICQQ |
| | chain | | STIM005 | SYGIPWVGQGTKVEIK |
| | variable | | | |
| | region | | | |
| 446 | STIM005 - | | Nucleic acid | GACATCCAGATGACCCAGTCTCCATCCTCCCTGTCTGCATCTGTA |
| | Light | | sequence of V_{L} of | GGAGACAGAGTCACCATCACTTGCCGGGCAAGTCAGAACATTAAT |
| | chain | | STIM005 | AACTTTTTAAATTGGTATCAGCAGAAAGAAGGGAAAGGCCCTAAG |
| | variable | | | CTCCTGATCTATGCAGCATCCAGTTTGCAAAGAGGGATACCATCA |
| | region | | | ACGTTCAGTGGCAGTGGATCTGGGACAGACTTCACTCTCACCATC |
| | | | | AGCAGTCTGCAACCTGAAGATTTTGCAACTTACATCTGTCAACAG |
| | | | | AGCTACGGTATCCCGTGGGTCGGCCAAGGGACCAAGGTGGAAATC |
| | | | | AAA |
| 447 | STIM005 - | | Amino acid | DIQMTQSPSSLSASVGDRVTITCRASQNINNFLNWYQQKEGKGPK |
| | full | | sequence of | LLIYAASSLQRGIPSTFSGSGSGTDFTLTISSLQPEDFATYICQQ |
| | light | | STIM005 light | SYGIPWVGQGTKVEIK |
| | chain | | chain | RTVAAPSVFIFPPSDEQLKSGTASVVCLLNNFYPREAKVQWKVDN |
| | sequence | | | ALQSGNSQESVTEQDSKDSTYSLSSTLTLSKADYEKHKVYACEVT |
| | | | | HQGLSSPVTKSFNRGEC |
| 448 | STIM005 - | | Nucleic acid | GACATCCAGATGACCCAGTCTCCATCCTCCCTGTCTGCATCTGTA |
| | full | | sequence of | GGAGACAGAGTCACCATCACTTGCCGGGCAAGTCAGAACATTAAT |
| | light | | STIM005 light | AACTTTTTAAATTGGTATCAGCAGAAAGAAGGGAAAGGCCCTAAG |
| | chain | | chain | CTCCTGATCTATGCAGCATCCAGTTTGCAAAGAGGGATACCATCA |
| | sequence | | | ACGTTCAGTGGCAGTGGATCTGGGACAGACTTCACTCTCACCATC |
| | | | | AGCAGTCTGCAACCTGAAGATTTTGCAACTTACATCTGTCAACAG |
| | | | | AGCTACGGTATCCCGTGGGTCGGCCAAGGGACCAAGGTGGAAATC |
| | | | | AAAcgtacggtggccgctccctccgtgttcatcttcccaccttcc |
| | | | | gacgagcagctgaagtccggcaccgcttctgtcgtgtgcctgctg |
| | | | | aacaacttctacccccgcgaggccaaggtgcagtggaaggtggac |
| | | | | aacgccctgcagtccggcaactcccaggaatccgtgaccgagcag |
| | | | | gactccaaggacagcacctactccctgtcctccaccctgaccctg |
| | | | | tccaaggccgactacgagaagcacaaggtgtacgcctgcgaagtg |
| | | | | acccaccagggcctgtctagccccgtgaccaagtctttcaaccgg |
| | | | | ggcgagtgt |
| 449 | STIM006 - | | Amino acid | GFTFSDYF |
| | CDRH1 | | sequence of CDRH1 | |
| | | | of STIM006 using | |
| | | | IMGT | |
| 450 | STIM006 - | | Amino acid | ISSSGSTI |
| | CDRH2 | | sequence of CDRH2 | |
| | | | of STIM006 using | |
| | | | IMGT | |
| 451 | STIM006 - | | Amino acid | ARDHYDGSGIYPLYYYYGLDV |
| | CDRH3 | | sequence of CDRH3 | |
| | | | of STIM006 using | |
| | | | IMGT | |
| 452 | STIM006 - | | Amino acid | QVQLVESGGGLVKPGGSLRLSCAASGFTFSDYFMSWIRQAPGKGL |
| | Heavy | | sequence of V_{H} of | EWISYISSSGSTIYYADSVRGRFTISRDNAKYSLYLQMNSLRSED |
| | chain | | STIM006 | TAVYYCARDHYDGSGIYPLYYYYGLDVWGQGTTVTVSS |
| | variable | | | |
| | region | | | |
| 453 | STIM006 - | | Nucleic acid | CAGGTGCAGCTGGTGGAGTCTGGGGGAGGCTTGGTCAAGCCTGGA |
| | Heavy | | sequence of V_{H} of | GGGTCCCTGAGACTCTCCTGTGCAGCCTCTGGATTCACCTTCAGT |
| | chain | | STIM006 | GACTACTTCATGAGCTGGATCCGCCAGGCGCCAGGGAAGGGGCTG |
| | variable | | | GAGTGGATTTCATACATTAGTTCTAGTGGTAGTACCATATACTAC |
| | region | | | GCAGACTCTGTGAGGGGCCGATTCACCATCTCCAGGGACAACGCC |
| | | | | AAGTACTCACTGTATCTGCAAATGAACAGCCTGAGATCCGAGGAC |
| | | | | ACGGCCGTGTATTACTGTGCGAGAGATCACTACGATGGTTCGGGG |
| | | | | ATTTATCCCCTCTACTACTATTACGGTTTGGACGTCTGGGGCCAG |
| | | | | GGGACCACGGTCACCGTCTCCTCA |
| 454 | STIM006 - | | Amino acid | QVQLVESGGGLVKPGGSLRLSCAASGFTFSDYFMSWIRQAPGKGL |
| | full | | sequence of | EWISYISSSGSTIYYADSVRGRFTISRDNAKYSLYLQMNSLRSED |
| | heavy | | STIM006 heavy | TAVYYCARDHYDGSGIYPLYYYYGLDVWGQGTTVTVSSASTKGPS |
| | chain | | chain | VFPLAPSSKSTSGGTAALGCLVKDYFPEPVTVSWNSGALTSGVHT |
| | sequence | | | FPAVLQSSGLYSLSSVVTVPSSSLGTQTYICNVNHKPSNTKVDKK |
| | | | | VEPKSCDKTHTCPPCPAPELLGGPSVFLFPPKPKDTLMISRTPEV |
| | | | | TCVVVDVSHEDPEVKFNWYVDGVEVHNAKTKPREEQYNSTYRVVS |
| | | | | VLTVLHQDWLNGKEYKCKVSNKALPAPIEKTISKAKGQPREPQVY |
| | | | | TLPPSRDELTKNQVSLTCLVKGFYPSDIAVEWESNGQPENNYKTT |
| | | | | PPVLDSDGSFFLYSKLTVDKSRWQQGNVFSCSVMHEALHNHYTQK |
| | | | | SLSLSPGK |
| 455 | STIM006 - | | Nucleic acid | CAGGTGCAGCTGGTGGAGTCTGGGGGAGGCTTGGTCAAGCCTGGA |
| | full | | sequence of | GGGTCCCTGAGACTCTCCTGTGCAGCCTCTGGATTCACCTTCAGT |
| | heavy | | STIM006 heavy | GACTACTTCATGAGCTGGATCCGCCAGGCGCCAGGGAAGGGGCTG |
| | chain | | chain | GAGTGGATTTCATACATTAGTTCTAGTGGTAGTACCATATACTAC |
| | sequence | | | GCAGACTCTGTGAGGGGCCGATTCACCATCTCCAGGGACAACGCC |
| | | | | AAGTACTCACTGTATCTGCAAATGAACAGCCTGAGATCCGAGGAC |
| | | | | ACGGCCGTGTATTACTGTGCGAGAGATCACTACGATGGTTCGGGG |
| | | | | ATTTATCCCCTCTACTACTATTACGGTTTGGACGTCTGGGGCCAG |
| | | | | GGGACCACGGTCACCGTCTCCTCAGCCAGCACCAAGGGCCCCTCT |
| | | | | GTGTTCCCTCTGGCCCCTTCCAGCAAGTCCACCTCTGGCGGAACA |
| | | | | GCCGCTCTGGGCTGCCTCGTGAAGGACTACTTCCCCGAGCCTGTG |
| | | | | ACCGTGTCCTGGAACTCTGGCGCTCTGACCAGCGGAGTGCACACC |
| | | | | TTCCCTGCTGTGCTGCAGTCCTCCGGCCTGTACTCCCTGTCCTCC |
| | | | | GTCGTGACCGTGCCTTCCAGCTCTCTGGGCACCCAGACCTACATC |
| | | | | TGCAACGTGAACCACAAGCCCTCCAACACCAAGGTGGACAAGAAG |
| | | | | GTGGAACCCAAGTCCTGCGACAAGACCCACACCTGTCCCCCTTGT |
| | | | | CCTGCCCCTGAACTGCTGGGCGGACCTTCCGTGTTCCTGTTCCCC |
| | | | | CCAAAGCCCAAGGACACCCTGATGATCTCCCGGACCCCCGAAGTG |
| | | | | ACCTGCGTGGTGGTGGATGTGTCCCACGAGGACCCTGAAGTGAAG |
| | | | | TTCAATTGGTACGTGGACGGCGTGGAAGTGCACAACGCCAAGACC |
| | | | | AAGCCTAGAGAGGAACAGTACAACTCCACCTACCGGGTGGTGTCC |
| | | | | GTGCTGACCGTGCTGCACCAGGATTGGCTGAACGGCAAAGAGTAC |
| | | | | AAGTGCAAGGTGTCCAACAAGGCCCTGCCTGCCCCCATCGAAAAG |
| | | | | ACCATCTCCAAGGCCAAGGGCCAGCCCCGGGAACCCCAGGTGTAC |
| | | | | ACACTGCCCCCTAGCAGGGACGAGCTGACCAAGAACCAGGTGTCC |
| | | | | CTGACCTGTCTCGTGAAAGGCTTCTACCCCTCCGATATCGCCGTG |
| | | | | GAATGGGAGTCCAACGGCCAGCCTGAGAACAACTACAAGACCACC |
| | | | | CCCCCTGTGCTGGACTCCGACGGCTCATTCTTCCTGTACAGCAAG |
| | | | | CTGACAGTGGACAAGTCCCGGTGGCAGCAGGGCAACGTGTTCTCC |
| | | | | TGCTCCGTGATGCACGAGGCCCTGCACAACCACTACACCCAGAAG |
| | | | | TCCCTGTCCCTGAGCCCCGGCAAGTGATGA |
| 456 | STIM006 - | | Amino acid | QSLLHSNGYNY |
| | CDRL1 | | sequence of CDRL1 | |
| | | | of STIM006 using | |
| | | | IMGT | |
| 457 | STIM006 - | | Amino acid | LGS |
| | CDRL2 | | sequence of CDRL2 | |
| | | | of STIM006 using | |
| | | | IMGT | |
| 458 | STIM006 - | | Amino acid | MQALQTPRS |
| | CDRL3 | | sequence of CDRL3 | |
| | | | of STIM006 using | |
| | | | IMGT | |
| 459 | STIM006 - | | Amino acid | IVMTQSPLSLPVTPGEPASISCRSSQSLLHSNGYNYLDYYLQKPG |
| | Light | | sequence of V_{L} of | QSPQLLIYLGSYRASGVPDRFSGSGSGTDFTLKISRVEAEDVGVY |
| | chain | | STIM006 | YCMQALQTPRSFGQGTTLEIK |
| | variable | | | |
| | region | | | |
| 460 | STIM006 - | | Nucleic acid | ATTGTGATGACTCAGTCTCCACTCTCCCTACCCGTCACCCCTGGA |
| | Light | | sequence of V_{L} of | GAGCCGGCCTCCATCTCCTGCAGGTCTAGTCAGAGCCTCCTGCAT |
| | chain | | STIM006 | AGTAATGGATACAACTATTTGGATTATTACCTGCAGAAGCCAGGG |
| | variable | | | CAGTCTCCACAGCTCCTGATCTATTTGGGTTCTTATCGGGCCTCC |
| | region | | | GGGGTCCCTGACAGGTTCAGTGGCAGTGGATCAGGCACAGATTTT |
| | | | | ACACTGAAAATCAGCAGAGTGGAGGCTGAGGATGTTGGGGTTTAT |
| | | | | TACTGCATGCAAGCTCTACAAACTCCTCGCAGTTTTGGCCAGGGG |
| | | | | ACCACGCTGGAGATCAAA |
| 461 | STIM006 - | | Amino acid | IVMTQSPLSLPVTPGEPASISCRSSQSLLHSNGYNYLDYYLQKPG |
| | full | | sequence of | QSPQLLIYLGSYRASGVPDRFSGSGSGTDFTLKISRVEAEDVGVY |
| | light | | STIM006 light | YCMQALQTPRSFGQGTTLEIKRTVAAPSVFIFPPSDEQLKSGTAS |
| | chain | | chain | VVCLLNNFYPREAKVQWKVDNALQSGNSQESVTEQDSKDSTYSLS |
| | sequence | | | STLTLSKADYEKHKVYACEVTHQGLSSPVTKSFNRGEC |
| 462 | STIM006 - | | Nucleic acid | ATTGTGATGACTCAGTCTCCACTCTCCCTACCCGTCACCCCTGGA |
| | full | | sequence of | GAGCCGGCCTCCATCTCCTGCAGGTCTAGTCAGAGCCTCCTGCAT |
| | light | | STIM006 light | AGTAATGGATACAACTATTTGGATTATTACCTGCAGAAGCCAGGG |
| | chain | | chain | CAGTCTCCACAGCTCCTGATCTATTTGGGTTCTTATCGGGCCTCC |
| | sequence | | | GGGGTCCCTGACAGGTTCAGTGGCAGTGGATCAGGCACAGATTTT |
| | | | | ACACTGAAAATCAGCAGAGTGGAGGCTGAGGATGTTGGGGTTTAT |
| | | | | TACTGCATGCAAGCTCTACAAACTCCTCGCAGTTTTGGCCAGGGG |
| | | | | ACCACGCTGGAGATCAAAcgtacggtggccgctccctccgtgttc |
| | | | | atcttcccaccttccgacgagcagctgaagtccggcaccgcttct |
| | | | | gtcgtgtgcctgctgaacaacttctacccccgcgaggccaaggtg |
| | | | | cagtggaaggtggacaacgccctgcagtccggcaactcccaggaa |
| | | | | tccgtgaccgagcaggactccaaggacagcacctactccctgtcc |
| | | | | tccaccctgaccctgtccaaggccgactacgagaagcacaaggtg |
| | | | | tacgcctgcgaagtgacccaccagggcctgtctagccccgtgacc |
| | | | | aagtctttcaaccggggcgagtgt |
| 463 | STIM007 - | | Amino acid | GFSLSTTGVG |
| | CDRH1 | | sequence of CDRH1 | |
| | | | of STIM007 using | |
| | | | IMGT | |
| 464 | STIM007 - | | Amino acid | IYWDDDK |
| | CDRH2 | | sequence of CDRH2 | |
| | | | of STIM007 using | |
| | | | IMGT | |
| 465 | STIM007 - | | Amino acid | THGYGSASYYHYGMDV |
| | CDRH3 | | sequence of CDRH3 | |
| | | | of STIM007 using | |
| | | | IMGT | |
| 466 | STIM007 - | | Amino acid | QITLKESGPTLVKPTQTLTLTCTFSGFSLSTTGVGVGWIRQPPGK |
| | Heavy | | sequence of V_{H} of | ALEWLAVIYWDDDKRYSPSLKSRLTITKDTSKNQVVLTMTNMDPV |
| | chain | | STIM007 | DTATYFCTHGYGSASYYHYGMDVWGQGTTVTVSS |
| | variable | | | |
| | region | | | |
| 467 | STIM007 - | | Nucleic acid | CAGATCACCTTGAAGGAGTCTGGTCCTACGCTGGTGAAACCCACA |
| | Heavy | | sequence of V_{H} of | CAGACCCTCACGCTGACCTGCACCTTCTCTGGGTTCTCACTCAGC |
| | chain | | STIM007 | ACTACTGGAGTGGGTGTGGGCTGGATCCGTCAGCCCCCAGGAAAG |
| | variable | | | GCCCTGGAGTGGCTTGCAGTCATTTATTGGGATGATGATAAGCGC |
| | region | | | TACAGCCCATCTCTGAAGAGCAGACTCACCATCACCAAGGACACC |
| | | | | TCCAAAAACCAGGTGGTCCTTACAATGACCAACATGGACCCTGTG |
| | | | | GACACAGCCACATATTTCTGTACACACGGATATGGTTCGGCGAGT |
| | | | | TATTACCACTACGGTATGGACGTCTGGGGCCAAGGGACCACGGTC |
| | | | | ACCGTCTCCTCA |
| 468 | STIM007 - | | Amino acid | QITLKESGPTLVKPTQTLTLTCTFSGFSLSTTGVGVGWIRQPPGK |
| | full | | sequence of | ALEWLAVIYWDDDKRYSPSLKSRLTITKDTSKNQVVLTMTNMDPV |
| | heavy | | STIM007 heavy | DTATYFCTHGYGSASYYHYGMDVWGQGTTVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 469 | STIM007 - | | Nucleic acid | CAGATCACCTTGAAGGAGTCTGGTCCTACGCTGGTGAAACCCACA |
| | full | | sequence of | CAGACCCTCACGCTGACCTGCACCTTCTCTGGGTTCTCACTCAGC |
| | heavy | | STIM007 heavy | ACTACTGGAGTGGGTGTGGGCTGGATCCGTCAGCCCCCAGGAAAG |
| | chain | | chain | GCCCTGGAGTGGCTTGCAGTCATTTATTGGGATGATGATAAGCGC |
| | sequence | | | TACAGCCCATCTCTGAAGAGCAGACTCACCATCACCAAGGACACC |
| | | | | TCCAAAAACCAGGTGGTCCTTACAATGACCAACATGGACCCTGTG |
| | | | | GACACAGCCACATATTTCTGTACACACGGATATGGTTCGGCGAGT |
| | | | | TATTACCACTACGGTATGGACGTCTGGGGCCAAGGGACCACGGTC |
| | | | | ACCGTCTCCTCA |
| | | | | GCCAGCACCAAGGGCCCCTCTGTGTTCCCTCTGGCCCCTTCCAGC |
| | | | | AAGTCCACCTCTGGCGGAACAGCCGCTCTGGGCTGCCTCGTGAAG |
| | | | | GACTACTTCCCCGAGCCTGTGACCGTGTCCTGGAACTCTGGCGCT |
| | | | | CTGACCAGCGGAGTGCACACCTTCCCTGCTGTGCTGCAGTCCTCC |
| | | | | GGCCTGTACTCCCTGTCCTCCGTCGTGACCGTGCCTTCCAGCTCT |
| | | | | CTGGGCACCCAGACCTACATCTGCAACGTGAACCACAAGCCCTCC |
| | | | | AACACCAAGGTGGACAAGAAGGTGGAACCCAAGTCCTGCGACAAG |
| | | | | ACCCACACCTGTCCCCCTTGTCCTGCCCCTGAACTGCTGGGCGGA |
| | | | | CCTTCCGTGTTCCTGTTCCCCCCAAAGCCCAAGGACACCCTGATG |
| | | | | ATCTCCCGGACCCCCGAAGTGACCTGCGTGGTGGTGGATGTGTCC |
| | | | | CACGAGGACCCTGAAGTGAAGTTCAATTGGTACGTGGACGGCGTG |
| | | | | GAAGTGCACAACGCCAAGACCAAGCCTAGAGAGGAACAGTACAAC |
| | | | | TCCACCTACCGGGTGGTGTCCGTGCTGACCGTGCTGCACCAGGAT |
| | | | | TGGCTGAACGGCAAAGAGTACAAGTGCAAGGTGTCCAACAAGGCC |
| | | | | CTGCCTGCCCCCATCGAAAAGACCATCTCCAAGGCCAAGGGCCAG |
| | | | | CCCCGGGAACCCCAGGTGTACACACTGCCCCCTAGCAGGGACGAG |
| | | | | CTGACCAAGAACCAGGTGTCCCTGACCTGTCTCGTGAAAGGCTTC |
| | | | | TACCCCTCCGATATCGCCGTGGAATGGGAGTCCAACGGCCAGCCT |
| | | | | GAGAACAACTACAAGACCACCCCCCCTGTGCTGGACTCCGACGGC |
| | | | | TCATTCTTCCTGTACAGCAAGCTGACAGTGGACAAGTCCCGGTGG |
| | | | | CAGCAGGGCAACGTGTTCTCCTGCTCCGTGATGCACGAGGCCCTG |
| | | | | CACAACCACTACACCCAGAAGTCCCTGTCCCTGAGCCCCGGCAAG |
| | | | | TGATGA |
| 470 | STIM007- | | Amino acid | QSVTNY |
| | CDRL1 | | sequence of CDRL1 | |
| | | | of STIM007 using | |
| | | | IMGT | |
| 471 | STIM007- | | Amino acid | DAS |
| | CDRL2 | | sequence of CDRL2 | |
| | | | of STIM007 using | |
| | | | IMGT | |
| 472 | STIM007- | | Amino acid | QHRSNWPLT |
| | CDRL3 | | sequence of CDRL3 | |
| | | | of STIM007 using | |
| | | | IMGT | |
| 473 | STIM007 - | | Amino acid | EIVLTQSPATLSLSPGERATLSCRASQSVTNYLAWHQQKPGQAPR |
| | Light | | sequence of V_{L} of | LLIYDASNRATGIPARFSGSGSGTDFTLTISSLEPEDFAVYYCQH |
| | chain | | STIM007 | RSNWPLTFGGGTKVEIK |
| | variable | | | |
| | region | | | |
| 474 | STIM007 - | | Nucleic acid | GAAATTGTATTGACACAGTCTCCAGCCACCCTGTCTTTGTCTCCA |
| | Light | | sequence of V_{L} of | GGGGAAAGAGCCACCCTCTCCTGCAGGGCCAGTCAGAGTGTTACC |
| | chain | | STIM007 | AACTACTTAGCCTGGCACCAACAGAAACCTGGCCAGGCTCCCAGG |
| | variable | | | CTCCTCATCTATGATGCATCCAACAGGGCCACTGGCATCCCAGCC |
| | region | | | AGGTTCAGTGGCAGTGGGTCTGGGACAGACTTCACTCTCACCATC |
| | | | | AGCAGCCTAGAGCCTGAAGATTTTGCAGTTTATTACTGTCAGCAC |
| | | | | CGTAGCAACTGGCCTCTCACTTTCGGCGGAGGGACCAAGGTGGAG |
| | | | | ATCAAAC |
| 475 | STIM007 - | | Amino acid | EIVLTQSPATLSLSPGERATLSCRASQSVTNYLAWHQQKPGQAPR |
| | full | | sequence of | LLIYDASNRATGIPARFSGSGSGTDFTLTISSLEPEDFAVYYCQH |
| | light | | STIM007 light | RSNWPLTFGGGTKVEIKRTVAAPSVFIFPPSDEQLKSGTASVVCL |
| | chain | | chain | LNNFYPREAKVQWKVDNALQSGNSQESVTEQDSKDSTYSLSSTLT |
| | sequence | | | LSKADYEKHKVYACEVTHQGLSSPVTKSFNRGEC |
| 476 | STIM007 - | | Nucleic acid | GAAATTGTATTGACACAGTCTCCAGCCACCCTGTCTTTGTCTCCA |
| | full | | sequence of | GGGGAAAGAGCCACCCTCTCCTGCAGGGCCAGTCAGAGTGTTACC |
| | light | | STIM007 light | AACTACTTAGCCTGGCACCAACAGAAACCTGGCCAGGCTCCCAGG |
| | chain | | chain | CTCCTCATCTATGATGCATCCAACAGGGCCACTGGCATCCCAGCC |
| | sequence | | | AGGTTCAGTGGCAGTGGGTCTGGGACAGACTTCACTCTCACCATC |
| | | | | AGCAGCCTAGAGCCTGAAGATTTTGCAGTTTATTACTGTCAGCAC |
| | | | | CGTAGCAACTGGCCTCTCACTTTCGGCGGAGGGACCAAGGTGGAG |
| | | | | ATCAAACcgtacggtggccgctccctccgtgttcatcttcccacc |
| | | | | ttccgacgagcagctgaagtccggcaccgcttctgtcgtgtgcct |
| | | | | gctgaacaacttctacccccgcgaggccaaggtgcagtggaaggt |
| | | | | ggacaacgccctgcagtccggcaactcccaggaatccgtgaccga |
| | | | | gcaggactccaaggacagcacctactccctgtcctccaccctgac |
| | | | | cctgtccaaggccgactacgagaagcacaaggtgtacgcctgcga |
| | | | | agtgacccaccagggcctgtctagccccgtgaccaagtctttcaa |
| | | | | ccggggcgagtgt |
| 477 | STIM008- | | Amino acid | GFSLSTSGVG |
| | CDRH1 | | sequence of CDRH1 | |
| | | | of STIM008 using | |
| | | | IMGT | |
| 478 | STIM008- | | Amino acid | IYWDDDK |
| | CDRH2 | | sequence of CDRH2 | |
| | | | of STIM008 using | |
| | | | IMGT | |
| 479 | STIM008- | | Amino acid | THGYGSASYYHYGMDV |
| | CDRH3 | | sequence of CDRH3 | |
| | | | of STIM008 using | |
| | | | IMGT | |
| 480 | STIM008 - | | Amino acid | QITLKESGPTLVKPTQTLTLTCTFSGFSLSTSGVGVGWIRQPPGK |
| | Heavy | | sequence of V_{H} of | ALEWLAVIYWDDDKRYSPSLKSRLTITKDTSKNQVVLTMTNMDPV |
| | chain | | STIM008 | DTATYFCTHGYGSASYYHYGMDVWGQGTTVTVSS |
| | variable | | | |
| | region | | | |
| 481 | STIM008 - | | Nucleic acid | CAGATCACCTTGAAGGAGTCTGGTCCTACGCTGGTGAAACCCACA |
| | Heavy | | sequence of V_{H} of | CAGACCCTCACGCTGACCTGCACCTTCTCTGGGTTCTCACTCAGC |
| | chain | | STIM008 | ACTAGTGGAGTGGGTGTGGGCTGGATCCGTCAGCCCCCAGGAAAG |
| | variable | | | GCCCTGGAGTGGCTTGCAGTCATTTATTGGGATGATGATAAGCGC |
| | region | | | TACAGCCCATCTCTGAAGAGCAGGCTCACCATCACCAAGGACACC |
| | | | | TCCAAAAACCAGGTGGTCCTTACAATGACCAACATGGACCCTGTG |
| | | | | GACACAGCCACATATTTCTGTACACACGGATATGGTTCGGCGAGT |
| | | | | TATTACCACTACGGTATGGACGTCTGGGGCCAAGGGACCACGGTC |
| | | | | ACCGTCTCCTCA |
| 482 | STIM008 - | | Amino acid | QITLKESGPTLVKPTQTLTLTCTFSGFSLSTSGVGVGWIRQPPGK |
| | full | | sequence of | ALEWLAVIYWDDDKRYSPSLKSRLTITKDTSKNQVVLTMTNMDPV |
| | heavy | | STIM008 heavy | DTATYFCTHGYGSASYYHYGMDVWGQGTTVTVSSASTKGPSVFPL |
| | chain | | chain | APSSKSTSGGTAALGCLVKDYFPEPVTVSWNSGALTSGVHTFPAV |
| | sequence | | | LQSSGLYSLSSVVTVPSSSLGTQTYICNVNHKPSNTKVDKKVEPK |
| | | | | SCDKTHTCPPCPAPELLGGPSVFLFPPKPKDTLMISRTPEVTCVV |
| | | | | VDVSHEDPEVKFNWYVDGVEVHNAKTKPREEQYNSTYRVVSVLTV |
| | | | | LHQDWLNGKEYKCKVSNKALPAPIEKTISKAKGQPREPQVYTLPP |
| | | | | SRDELTKNQVSLTCLVKGFYPSDIAVEWESNGQPENNYKTTPPVL |
| | | | | DSDGSFFLYSKLTVDKSRWQQGNVFSCSVMHEALHNHYTQKSLSL |
| | | | | SPGK |
| 483 | STIM008 - | | Nucleic acid | CAGATCACCTTGAAGGAGTCTGGTCCTACGCTGGTGAAACCCACA |
| | full | | sequence of | CAGACCCTCACGCTGACCTGCACCTTCTCTGGGTTCTCACTCAGC |
| | heavy | | STIM008 heavy | ACTAGTGGAGTGGGTGTGGGCTGGATCCGTCAGCCCCCAGGAAAG |
| | chain | | chain | GCCCTGGAGTGGCTTGCAGTCATTTATTGGGATGATGATAAGCGC |
| | sequence | | | TACAGCCCATCTCTGAAGAGCAGGCTCACCATCACCAAGGACACC |
| | | | | TCCAAAAACCAGGTGGTCCTTACAATGACCAACATGGACCCTGTG |
| | | | | GACACAGCCACATATTTCTGTACACACGGATATGGTTCGGCGAGT |
| | | | | TATTACCACTACGGTATGGACGTCTGGGGCCAAGGGACCACGGTC |
| | | | | ACCGTCTCCTCAGCCAGCACCAAGGGCCCCTCTGTGTTCCCTCTG |
| | | | | GCCCCTTCCAGCAAGTCCACCTCTGGCGGAACAGCCGCTCTGGGC |
| | | | | TGCCTCGTGAAGGACTACTTCCCCGAGCCTGTGACCGTGTCCTGG |
| | | | | AACTCTGGCGCTCTGACCAGCGGAGTGCACACCTTCCCTGCTGTG |
| | | | | CTGCAGTCCTCCGGCCTGTACTCCCTGTCCTCCGTCGTGACCGTG |
| | | | | CCTTCCAGCTCTCTGGGCACCCAGACCTACATCTGCAACGTGAAC |
| | | | | CACAAGCCCTCCAACACCAAGGTGGACAAGAAGGTGGAACCCAAG |
| | | | | TCCTGCGACAAGACCCACACCTGTCCCCCTTGTCCTGCCCCTGAA |
| | | | | CTGCTGGGCGGACCTTCCGTGTTCCTGTTCCCCCCAAAGCCCAAG |
| | | | | GACACCCTGATGATCTCCCGGACCCCCGAAGTGACCTGCGTGGTG |
| | | | | GTGGATGTGTCCCACGAGGACCCTGAAGTGAAGTTCAATTGGTAC |
| | | | | GTGGACGGCGTGGAAGTGCACAACGCCAAGACCAAGCCTAGAGAG |
| | | | | GAACAGTACAACTCCACCTACCGGGTGGTGTCCGTGCTGACCGTG |
| | | | | CTGCACCAGGATTGGCTGAACGGCAAAGAGTACAAGTGCAAGGTG |
| | | | | TCCAACAAGGCCCTGCCTGCCCCCATCGAAAAGACCATCTCCAAG |
| | | | | GCCAAGGGCCAGCCCCGGGAACCCCAGGTGTACACACTGCCCCCT |
| | | | | AGCAGGGACGAGCTGACCAAGAACCAGGTGTCCCTGACCTGTCTC |
| | | | | GTGAAAGGCTTCTACCCCTCCGATATCGCCGTGGAATGGGAGTCC |
| | | | | AACGGCCAGCCTGAGAACAACTACAAGACCACCCCCCCTGTGCTG |
| | | | | GACTCCGACGGCTCATTCTTCCTGTACAGCAAGCTGACAGTGGAC |
| | | | | AAGTCCCGGTGGCAGCAGGGCAACGTGTTCTCCTGCTCCGTGATG |
| | | | | CACGAGGCCCTGCACAACCACTACACCCAGAAGTCCCTGTCCCTG |
| | | | | AGCCCCGGCAAGTGATGA |
| 484 | STIM008- | | Amino acid | QSVTNY |
| | CDRL1 | | sequence of CDRL1 | |
| | | | of STIM008 using | |
| | | | IMGT | |
| 485 | STIM008- | | Amino acid | DAS |
| | CDRL2 | | sequence of CDRL2 | |
| | | | of STIM008 using | |
| | | | IMGT | |
| 486 | STIM008- | | Amino acid | QQRSNWPLT |
| | CDRL3 | | sequence of CDRL3 | |
| | | | of STIM008 using | |
| | | | IMGT | |
| 487 | STIM008 - | | Amino acid | EIVLTQSPATLSLSPGERATLSCRASQSVTNYLAWHQQKPGQAPR |
| | Light | | sequence of V_{L} of | LLIYDASNRATGIPARFSGSGSGTDFTLTISSLEPEDFAVYYCQQ |
| | chain | | STIM008 | RSNWPLTFGGGTKVEIK |
| | variable | | | |
| | region | | | |
| 488 | STIM008 - | | Nucleic acid | GAAATTGTGTTGACACAGTCTCCAGCCACCCTGTCTTTGTCTCCA |
| | Light | | sequence of V_{L} of | GGGGAAAGAGCCACCCTCTCCTGCAGGGCCAGTCAGAGTGTTACC |
| | chain | | STIM008 | AACTACTTAGCCTGGCACCAACAGAAACCTGGCCAGGCTCCCAGG |
| | variable | | | CTCCTCATCTATGATGCATCCAACAGGGCCACTGGCATCCCAGCC |
| | region | | | AGGTTCAGTGGCAGTGGGTCTGGGACAGACTTCACTCTCACCATC |
| | | | | AGCAGCCTAGAGCCTGAAGATTTTGCAGTTTATTACTGTCAGCAG |
| | | | | CGTAGCAACTGGCCTCTCACTTTCGGCGGAGGGACCAAGGTGGAG |
| | | | | ATCAAA |
| 489 | STIM008 - | | Amino acid | EIVLTQSPATLSLSPGERATLSCRASQSVTNYLAWHQQKPGQAPR |
| | full | | sequence of | LLIYDASNRATGIPARFSGSGSGTDFTLTISSLEPEDFAVYYCQQ |
| | light | | STIM008 light | RSNWPLTFGGGTKVEIKRTVAAPSVFIFPPSDEQLKSGTASVVCL |
| | chain | | chain | LNNFYPREAKVQWKVDNALQSGNSQESVTEQDSKDSTYSLSSTLT |
| | sequence | | | LSKADYEKHKVYACEVTHQGLSSPVTKSFNRGEC |
| 490 | STIM008 - | | Nucleic acid | GAAATTGTGTTGACACAGTCTCCAGCCACCCTGTCTTTGTCTCCA |
| | full | | sequence of | GGGGAAAGAGCCACCCTCTCCTGCAGGGCCAGTCAGAGTGTTACC |
| | light | | STIM008 light | AACTACTTAGCCTGGCACCAACAGAAACCTGGCCAGGCTCCCAGG |
| | chain | | chain | CTCCTCATCTATGATGCATCCAACAGGGCCACTGGCATCCCAGCC |
| | sequence | | | AGGTTCAGTGGCAGTGGGTCTGGGACAGACTTCACTCTCACCATC |
| | | | | AGCAGCCTAGAGCCTGAAGATTTTGCAGTTTATTACTGTCAGCAG |
| | | | | CGTAGCAACTGGCCTCTCACTTTCGGCGGAGGGACCAAGGTGGAG |
| | | | | ATCAAAcgtacggtggccgctccctccgtgttcatcttcccacct |
| | | | | tccgacgagcagctgaagtccggcaccgcttctgtcgtgtgcctg |
| | | | | ctgaacaacttctacccccgcgaggccaaggtgcagtggaaggtg |
| | | | | gacaacgccctgcagtccggcaactcccaggaatccgtgaccgag |
| | | | | caggactccaaggacagcacctactccctgtcctccaccctgacc |
| | | | | ctgtccaaggccgactacgagaagcacaaggtgtacgcctgcgaa |
| | | | | gtgacccaccagggcctgtctagccccgtgaccaagtctttcaac |
| | | | | cggggcgagtgt |
| 491 | STIM009- | | Amino acid | GFTFSDYY |
| | CDRH1 | | sequence of CDRH1 | |
| | | | of STIM009 using | |
| | | | IMGT | |
| 492 | STIM009- | | Amino acid | ISSSGSTI |
| | CDRH2 | | sequence of CDRH2 | |
| | | | of STIM009 using | |
| | | | IMGT | |
| 493 | STIM009- | | Amino acid | ARDFYDILTDSPYFYYGVDV |
| | CDRH3 | | sequence of CDRH3 | |
| | | | of STIM009 using | |
| | | | IMGT | |
| 494 | STIM009 - | | Amino acid | QVQLVESGGGLVKPGGSLRLSCAASGFTFSDYYMSWIRQAPGKGL |
| | Heavy | | sequence of V_{H} of | EWVSYISSSGSTIYYADSVKGRFTISRDNAKNSLYLQINSLRAED |
| | chain | | STIM009 | TAVYYCARDFYDILTDSPYFYYGVDVWGQGTTVTVSS |
| | variable | | | |
| | region | | | |
| 495 | STIM009 - | | Nucleic acid | CAGGTGCAGCTGGTGGAGTCTGGGGGAGGCTTGGTCAAGCCTGGA |
| | Heavy | | sequence of V_{H} of | GGGTCCCTGAGACTCTCCTGTGCAGCCTCTGGATTCACCTTCAGT |
| | chain | | STIM009 | GACTACTACATGAGCTGGATCCGCCAGGCTCCAGGGAAGGGGCTG |
| | variable | | | GAGTGGGTTTCATACATTAGTAGTAGTGGTAGTACCATATACTAC |
| | region | | | GCAGACTCTGTGAAGGGCCGATTCACCATCTCCAGGGACAACGCC |
| | | | | AAGAACTCACTGTATCTGCAAATTAACAGCCTGAGAGCCGAGGAC |
| | | | | ACGGCCGTGTATTACTGTGCGAGAGATTTTTACGATATTTTGACT |
| | | | | GATAGTCCGTACTTCTACTACGGTGTGGACGTCTGGGGCCAAGGG |
| | | | | ACCACGGTCACCGTCTCCTCA |
| 496 | STIM009 - | | Amino acid | QVQLVESGGGLVKPGGSLRLSCAASGFTFSDYYMSWIRQAPGKGL |
| | full | | sequence of | EWVSYISSSGSTIYYADSVKGRFTISRDNAKNSLYLQINSLRAED |
| | heavy | | STIM009 heavy | TAVYYCARDFYDILTDSPYFYYGVDVWGQGTTVTVSSASTKGPSV |
| | chain | | chain | FPLAPSSKSTSGGTAALGCLVKDYFPEPVTVSWNSGALTSGVHTF |
| | sequence | | | PAVLQSSGLYSLSSVVTVPSSSLGTQTYICNVNHKPSNTKVDKKV |
| | | | | EPKSCDKTHTCPPCPAPELLGGPSVFLFPPKPKDTLMISRTPEVT |
| | | | | CVVVDVSHEDPEVKFNWYVDGVEVHNAKTKPREEQYNSTYRVVSV |
| | | | | LTVLHQDWLNGKEYKCKVSNKALPAPIEKTISKAKGQPREPQVYT |
| | | | | LPPSRDELTKNQVSLTCLVKGFYPSDIAVEWESNGQPENNYKTTP |
| | | | | PVLDSDGSFFLYSKLTVDKSRWQQGNVFSCSVMHEALHNHYTQKS |
| | | | | LSLSPGK |
| 497 | STIM009 - | | Nucleic acid | CAGGTGCAGCTGGTGGAGTCTGGGGGAGGCTTGGTCAAGCCTGGA |
| | full | | sequence of | GGGTCCCTGAGACTCTCCTGTGCAGCCTCTGGATTCACCTTCAGT |
| | heavy | | STIM009 heavy | GACTACTACATGAGCTGGATCCGCCAGGCTCCAGGGAAGGGGCTG |
| | chain | | chain | GAGTGGGTTTCATACATTAGTAGTAGTGGTAGTACCATATACTAC |
| | sequence | | | GCAGACTCTGTGAAGGGCCGATTCACCATCTCCAGGGACAACGCC |
| | | | | AAGAACTCACTGTATCTGCAAATTAACAGCCTGAGAGCCGAGGAC |
| | | | | ACGGCCGTGTATTACTGTGCGAGAGATTTTTACGATATTTTGACT |
| | | | | GATAGTCCGTACTTCTACTACGGTGTGGACGTCTGGGGCCAAGGG |
| | | | | ACCACGGTCACCGTCTCCTCAGCCAGCACCAAGGGCCCCTCTGTG |
| | | | | TTCCCTCTGGCCCCTTCCAGCAAGTCCACCTCTGGCGGAACAGCC |
| | | | | GCTCTGGGCTGCCTCGTGAAGGACTACTTCCCCGAGCCTGTGACC |
| | | | | GTGTCCTGGAACTCTGGCGCTCTGACCAGCGGAGTGCACACCTTC |
| | | | | CCTGCTGTGCTGCAGTCCTCCGGCCTGTACTCCCTGTCCTCCGTC |
| | | | | GTGACCGTGCCTTCCAGCTCTCTGGGCACCCAGACCTACATCTGC |
| | | | | AACGTGAACCACAAGCCCTCCAACACCAAGGTGGACAAGAAGGTG |
| | | | | GAACCCAAGTCCTGCGACAAGACCCACACCTGTCCCCCTTGTCCT |
| | | | | GCCCCTGAACTGCTGGGCGGACCTTCCGTGTTCCTGTTCCCCCCA |
| | | | | AAGCCCAAGGACACCCTGATGATCTCCCGGACCCCCGAAGTGACC |
| | | | | TGCGTGGTGGTGGATGTGTCCCACGAGGACCCTGAAGTGAAGTTC |
| | | | | AATTGGTACGTGGACGGCGTGGAAGTGCACAACGCCAAGACCAAG |
| | | | | CCTAGAGAGGAACAGTACAACTCCACCTACCGGGTGGTGTCCGTG |
| | | | | CTGACCGTGCTGCACCAGGATTGGCTGAACGGCAAAGAGTACAAG |
| | | | | TGCAAGGTGTCCAACAAGGCCCTGCCTGCCCCCATCGAAAAGACC |
| | | | | ATCTCCAAGGCCAAGGGCCAGCCCCGGGAACCCCAGGTGTACACA |
| | | | | CTGCCCCCTAGCAGGGACGAGCTGACCAAGAACCAGGTGTCCCTG |
| | | | | ACCTGTCTCGTGAAAGGCTTCTACCCCTCCGATATCGCCGTGGAA |
| | | | | TGGGAGTCCAACGGCCAGCCTGAGAACAACTACAAGACCACCCCC |
| | | | | CCTGTGCTGGACTCCGACGGCTCATTCTTCCTGTACAGCAAGCTG |
| | | | | ACAGTGGACAAGTCCCGGTGGCAGCAGGGCAACGTGTTCTCCTGC |
| | | | | TCCGTGATGCACGAGGCCCTGCACAACCACTACACCCAGAAGTCC |
| | | | | CTGTCCCTGAGCCCCGGCAAGTGATGA |
| 498 | STIM009- | | Amino acid | QSLLHSNGYNY |
| | CDRL1 | | sequence of CDRL1 | |
| | | | of STIM009 using | |
| | | | IMGT | |
| 499 | STIM009- | | Amino acid | LGS |
| | CDRL2 | | sequence of CDRL2 | |
| | | | of STIM009 using | |
| | | | IMGT | |
| 500 | STIM009- | | Amino acid | MQALQTPRT |
| | CDRL3 | | sequence of CDRL3 | |
| | | | of STIM009 using | |
| | | | IMGT | |
| 501 | STIM009 - | | Amino acid | DIVMTQSPLSLPVTPGEPASISCRSSQSLLHSNGYNYLDWYLQKP |
| | Light | | sequence of V_{L} of | GQSPQLLIYLGSNRASGVPDRFSGSGSGTDFTLKISRVEAEDVGV |
| | chain | | STIM009 | YYCMQALQTPRTFGQGTKVEIK |
| | variable | | | |
| | region | | | |
| 502 | STIM009 - | | Nucleic acid | GATATTGTGATGACTCAGTCTCCACTCTCCCTGCCCGTCACCCCT |
| | Light | | sequence of V_{L} of | GGAGAGCCGGCCTCCATCTCCTGCAGGTCTAGTCAGAGCCTCCTG |
| | chain | | STIM009 | CATAGTAATGGATACAACTATTTGGATTGGTACCTGCAGAAGCCA |
| | variable | | | GGGCAGTCTCCACAGCTCCTGATCTATTTGGGTTCTAATCGGGCC |
| | region | | | TCCGGGGTCCCTGACAGGTTCAGTGGCAGTGGATCAGGCACAGAT |
| | | | | TTTACACTGAAAATCAGCAGAGTGGAGGCTGAGGATGTTGGGGTT |
| | | | | TATTACTGCATGCAAGCTCTACAAACTCCTCGGACGTTCGGCCAA |
| | | | | GGGACCAAGGTGGAAATCAAA |
| 503 | STIM009 - | | Amino acid | DIVMTQSPLSLPVTPGEPASISCRSSQSLLHSNGYNYLDWYLQKP |
| | full | | sequence of | GQSPQLLIYLGSNRASGVPDRFSGSGSGTDFTLKISRVEAEDVGV |
| | light | | STIM009 light | YYCMQALQTPRTFGQGTKVEIKRTVAAPSVFIFPPSDEQLKSGTA |
| | chain | | chain | SVVCLLNNFYPREAKVQWKVDNALQSGNSQESVTEQDSKDSTYSL |
| | sequence | | | SSTLTLSKADYEKHKVYACEVTHQGLSSPVTKSFNRGEC |
| 504 | STIM009 - | | Nucleic acid | GATATTGTGATGACTCAGTCTCCACTCTCCCTGCCCGTCACCCCT |
| | full | | sequence of | GGAGAGCCGGCCTCCATCTCCTGCAGGTCTAGTCAGAGCCTCCTG |
| | light | | STIM009 light | CATAGTAATGGATACAACTATTTGGATTGGTACCTGCAGAAGCCA |
| | chain | | chain | GGGCAGTCTCCACAGCTCCTGATCTATTTGGGTTCTAATCGGGCC |
| | sequence | | | TCCGGGGTCCCTGACAGGTTCAGTGGCAGTGGATCAGGCACAGAT |
| | | | | TTTACACTGAAAATCAGCAGAGTGGAGGCTGAGGATGTTGGGGTT |
| | | | | TATTACTGCATGCAAGCTCTACAAACTCCTCGGACGTTCGGCCAA |
| | | | | GGGACCAAGGTGGAAATCAAAcgtacggtggccgctccctccgtg |
| | | | | ttcatcttcccaccttccgacgagcagctgaagtccggcaccgct |
| | | | | tctgtcgtgtgcctgctgaacaacttctacccccgcgaggccaag |
| | | | | gtgcagtggaaggtggacaacgccctgcagtccggcaactcccag |
| | | | | gaatccgtgaccgagcaggactccaaggacagcacctactccctg |
| | | | | tcctccaccctgaccctgtccaaggccgactacgagaagcacaag |
| | | | | gtgtacgcctgcgaagtgacccaccagggcctgtctagccccgtg |
| | | | | accaagtctttcaaccggggcgagtgt |
| 505 | Human | | Amino acid | FTVTVPKDLYVVEYGSNMTIECKFPVEKQLDLAALIVYWEMEDKN |
| | PD-L1 | | sequence of | IIQFVHGEEDLKVQHSSYRQRARLLKDQLSLGNAALQITDVKLQD |
| | Flag His | | KYPROT286 with | AGVYRCMISYGGADYKRITVKVNAPYNKINQRILVVDPVTSEHEL |
| | (KYPROT2 | | FLAG tag in bold | TCQAEGYPKAEVIWTSSDHQVLSGKTTTTNSKREEKLFNVTSTLR |
| | 86) | | and underlined and | INTTTNEIFYCTFRRLDPEENHTAELVIPELPLAHPPNERTIEGR |
| | | | histidine tag in | DYKDDDDKHHHHHH |
| | | | bold | |
| 506 | Mature | | Mature amino acid | EINGSANYEMFIFHNGGVQILCKYPDIVQQFKMQLLKGGQILCDL |
| | human | | sequence of human | TKTKGSGNTVSIKSLKFCHSQLSNNSVSFFLYNLDHSHANYYFCN |
| | ICOS | | ICOS | LSIFDPPPFKVTLTGGYLHIYESQLCCQLKFWLPIGCAAFVVVCI |
| | | | | LGCILICWLTKKKYSSSVHDPNGEYMEMRAVNTAKKSRLTDVTL |
| 507 | Human | | Amino acid | EINGSANYEMFIFHNGGVQILCKYPDIVQQFKMQLLKGGQILCDL |
| | ICOS | | sequence of human | TKTKGSGNTVSIKSLKFCHSQLSNNSVSFFLYNLDHSHANYYFCN |
| | extracellular | | ICOS extracellular | LSIFDPPPFKVTLTGGYLHIYESQLCCQLKF |
| | domain | | domain | |
| 508 | Human | | Amino acid | MKSGLWYFFLFCLRIKVLTGEINGSANYEMFIFHNGGVQILCKYP |
| | ICOS | | sequence of human | DIVQQFKMQLLKGGQILCDLTKTKGSGNTVSIKSLKFCHSQLSNN |
| | with | | ICOS (signal | SVSFFLYNLDHSHANYYFCNLSIFDPPPFKVTLTGGYLHIYESQL |
| | signal | | peptide is | CCQLKFWLPIGCAAFVVVCILGCILICWLTKKKYSSSVHDPNGEY |
| | peptide | | underlined) | MFMRAVNTAKKSRLTDVTL |
| 509 | Isoform | | Amino acid | The sequence of this isoform differs from the |
| | of human | | sequence of a | canonical sequence in its cytoplasmic domain |
| | ICOS | | human ICOS isoform | as follows: 168-199: |
| | (Q9Y6W8-2) | | | KYSSSVHDPNGEYMFMRAVNTAKKSRLTDVTLM |
| 510 | Mature | | Amino acid | EINGSADHRMFSFHNGGVQISCKYPETVQQLKMRLFREREVLCEL |
| | mouse | | sequence of mature | TKTKGSGNAVSIKNPMLCLYHLSNNSVSFFLNNPDSSQGSYYFCS |
| | ICOS | | mouse ICOS | LSIFDPPPFQERNLSGGYLHIYESQLCCQLKIVVQVTE |
| 511 | Mouse | | Amino acid | EINGSADHRMFSFHNGGVQISCKYPETVQQLKMRLFREREVLCEL |
| | ICOS | | sequence of the | TKTKGSGNAVSIKNPMLCLYHLSNNSVSFFLNNPDSSQGSYYFCS |
| | extracellular | | extracellular | LSIFDPPPFQERNLSGGYLHIYESQLCCQLK |
| | domain | | domain of mouse | |
| | | | ICOS | |
| 512 | Mouse | | Amino acid | MGWSCIILFLVATATGVHSEINGSADHRMFSFHNGGVQISCKYPE |
| | ICOS | | sequence of mouse | TVQQLKMRLFREREVLCELTKTKGSGNAVSIKNPMLCLYHLSNNS |
| | with | | ICOS (signal | VSFFLNNPDSSQGSYYFCSLSIFDPPPFQERNLSGGYLHIYESQL |
| | signal | | peptide is | CCQLKIVVQVTE |
| | peptide | | underlined) | |
| 513 | Cynomolgus | | Amino acid | MKSGLWYFFL FCLHMKVLTG EINGSANYEM FIFHNGGVQI |
| | ICOS | | sequence of | LCKYPDIVQQ |
| | with | | cynomolgus ICOS | FKMQLLKGGQILCDLTKTKGSGNKVSIKSLKFCHSQLSNNSVSFF |
| | signal | | (signal peptide is | LYNLD |
| | peptide | | underlined) | RSHANYYFCNLSIFDPPPFKVTLTGGYLHIYESQLCCQLKFWLPI |
| | | | | GCATF |
| | | | | VVVCIFGCILICWLTKKKYSSTVHDPNGEYMFMRAVNTAKKSRLT |
| | | | | GTTP |
| 514 | Cynomolgus | | Amino acid | EINGSANYEMFIFHNGGVQILCKYPDIVQQFKMQLLKGGQILCDL |
| | ICOS | | sequence of | TKTKG |
| | extracellular | | cynomolgus ICOS | SGNKVSIKSLKFCHSQLSNNSVSFFLYNLDRSHANYYFCNLSIFD |
| | domain | | extracellular | PPPFK VTLTGGYLHIYESQLCCQLK |
| | | | domain | |
| 515 | Human | | Amino acid | DTQEKEVRAMVGSDVELSCACPEGSRFDLNDVYVYWQTSESKTVV |
| | ICOS | | sequence of human | TYHIPQNSSLENVDSRYRNRALMSPAGMLRGDFSLRLFNVTPQDE |
| | ligand | | ICOS ligand | QKFHCLVLSQSLGFQEVLSVEVTLHVAANFSVPVVSAPHSPSQDE |
| | | | comprising | LTFTCTSINGYPRPNVYWINKTDNSLLDQALQNDTVFLNMRGLYD |
| | | | extracellular | VVSVLRIARTPSVNIGCCIENVLLQQNLTVGSQTGNDIGERDKIT |
| | | | domain | ENPVSTGEKNAATWS |
| 516 | Human | | Amino acid | MRLGSPGLLFLLFSSLRADTQEKEVRAMVGSDVELSCACPEGSRF |
| | ICOS | | sequence of human | DLNDVYVYWQTSESKTVVTYHIPQNSSLENVDSRYRNRALMSPAG |
| | ligand | | ICOS ligand | MLRGDFSLRLFNVTPQDEQKFHCLVLSQSLGFQEVLSVEVTLHVA |
| | | | including signal | ANFSVPVVSAPHSPSQDELTFTCTSINGYPRPNVYWINKTDNSLL |
| | | | peptide | DQALQNDTVFLNMRGLYDVVSVLRIARTPSVNIGCCIENVLLQQN |
| | | | | LTVGSQTGNDIGERDKITENPVSTGEKNAATWSILAVLCLLVVVA |
| | | | | VAIGWVCRDRCLQHSYAGAWAVSPETELTGHV |
| 517 | C-terminal amino | | Amino acids 21 to | LQMILNGINNYKNPKLTAMLTFKFYMPKKATELKHLQC |
| | acid sequence of | | 133 of hIL-2 with | LEEELKPLEEVLNLAQSKNFHLRPRDLISNINVIVLEL |
| | hIL-2 | | R38W mutation | KGSETTFMCEYADETATIVEFLNRWITFCQSIISTLT |
| | | | (bold & | |
| | | | underlined) | |
| 518 | C-terminal amino | | Amino acids 21 to | LQMILNGINNYKNPKLTQMLTFKFYMPKKATELKHLQC |
| | acid sequence of | | 133 of hIL-2 with | LEEELKPLEEVLNLAQSKNFHLRPRDLISNINVIVLEL |
| | hIL-2 | | R38Q mutation | KGSETTFMCEYADETATIVEFLNRWITFCQSIISTLT |
| | | | (bold & | |
| | | | underlined) | |
| 519 | STIM002 - | | Nucleic acid | GATATTGTGATGACTCAGTCTCCACTCTCCCTGCCCGT |
| | Corrected Light | | sequence of | CACCCCTGGAGAGCCGGCCTCCATCTCCTGCAGGTCTA |
| | chain variable | | corrected V_{L} of | GTCAGAGCCTCCTGCATAGTGATGGATACAACTATTTG |
| | region | | STIM002 | GATTGGTACCTGCAGAAGCCAGGGCAGTCTCCACAGCT |
| | | | | CCTGATCTATTTGGGTTCTACTCGGGCCTCCGGGTTCC |
| | | | | CTGACAGGTTCAGTGGCAGTGGATCAGGCACAGATTTT |
| | | | | ACACTGAAAATCAGCAGAGTGGAGGCTGAGGATGTTGG |
| | | | | GGTTTATTACTGCATGCAAGCTCTACAAACTCCGCTCA |
| | | | | GTTTTGGCCAGGGGACCAAGCTGGAGATCAAA |
| 520 | STIM002 - | | Nucleic acid | GATATTGTGATGACTCAGTCTCCACTCTCCCTGCCCGT |
| | Corrected full | | sequence of | CACCCCTGGAGAGCCGGCCTCCATCTCCTGCAGGTCTA |
| | light chain | | corrected STIM002 | GTCAGAGCCTCCTGCATAGTGATGGATACAACTATTTG |
| | sequence | | light chain | GATTGGTACCTGCAGAAGCCAGGGCAGTCTCCACAGCT |
| | | | | CCTGATCTATTTGGGTTCTACTCGGGCCTCCGGGTTCC |
| | | | | CTGACAGGTTCAGTGGCAGTGGATCAGGCACAGATTTT |
| | | | | ACACTGAAAATCAGCAGAGTGGAGGCTGAGGATGTTGG |
| | | | | GGTTTATTACTGCATGCAAGCTCTACAAACTCCGCTCA |
| | | | | GTTTTGGCCAGGGGACCAAGCTGGAGATCAAAcgtacg |
| | | | | gtggccgctccctccgtgttcatcttcccaccttccga |
| | | | | cgagcagctgaagtccggcaccgcttctgtcgtgtgcc |
| | | | | tgctgaacaacttctacccccgcgaggccaaggtgcag |
| | | | | tggaaggtggacaacgccctgcagtccggcaactccca |
| | | | | ggaatccgtgaccgagcaggactccaaggacagcacct |
| | | | | actccctgtcctccaccctgaccctgtccaaggccgac |
| | | | | tacgagaagcacaaggtgtacgcctgcgaagtgaccca |
| | | | | ccagggcctgtctagccccgtgaccaagtctttcaacc |
| | | | | ggggcgagtgt |
| 521 | STIM003 - | | Nucleic acid | GAGGTGCAGCTGGTGGAGTCTGGGGGAGGTGTGGTACG |
| | Corrected heavy | | sequence of | GCCTGGGGGGTCCCTGAGACTCTCCTGTGTAGCCTCTG |
| | chain variable | | corrected VH of | GAGTCACCTTTGATGATTATGGCATGAGCTGGGTCCGC |
| | region | | STIM003 | CAAGCTCCAGGGAAGGGGCTGGAGTGGGTCTCTGGTAT |
| | | | | TAATTGGAATGGTGGCGACACAGATTATTCAGACTCTG |
| | | | | TGAAGGGCCGATTCACCATCTCCAGAGACAACGCCAAG |
| | | | | AACTCCCTGTATCTACAAATGAATAGTCTGAGAGCCGA |
| | | | | GGACACGGCCTTGTATTACTGTGCGAGGGATTTCTATG |
| | | | | GTTCGGGGAGTTATTATCACGTTCCTTTTGACTACTGG |
| | | | | GGCCAGGGAATCCTGGTCACCGTCTCCTCA |
| 522 | STIM003 - | | Nucleic acid | GAGGTGCAGCTGGTGGAGTCTGGGGGAGGTGTGGTACG |
| | Corrected full | | sequence of | GCCTGGGGGGTCCCTGAGACTCTCCTGTGTAGCCTCTG |
| | heavy chain | | corrected STIM003 | GAGTCACCTTTGATGATTATGGCATGAGCTGGGTCCGC |
| | sequence | | heavy chain | CAAGCTCCAGGGAAGGGGCTGGAGTGGGTCTCTGGTAT |
| | | | | TAATTGGAATGGTGGCGACACAGATTATTCAGACTCTG |
| | | | | TGAAGGGCCGATTCACCATCTCCAGAGACAACGCCAAG |
| | | | | AACTCCCTGTATCTACAAATGAATAGTCTGAGAGCCGA |
| | | | | GGACACGGCCTTGTATTACTGTGCGAGGGATTTCTATG |
| | | | | GTTCGGGGAGTTATTATCACGTTCCTTTTGACTACTGG |
| | | | | GGCCAGGGAATCCTGGTCACCGTCTCCTCAGCCAGCAC |
| | | | | CAAGGGCCCCTCTGTGTTCCCTCTGGCCCCTTCCAGCA |
| | | | | AGTCCACCTCTGGCGGAACAGCCGCTCTGGGCTGCCTC |
| | | | | GTGAAGGACTACTTCCCCGAGCCTGTGACCGTGTCCTG |
| | | | | GAACTCTGGCGCTCTGACCAGCGGAGTGCACACCTTCC |
| | | | | CTGCTGTGCTGCAGTCCTCCGGCCTGTACTCCCTGTCC |
| | | | | TCCGTCGTGACCGTGCCTTCCAGCTCTCTGGGCACCCA |
| | | | | GACCTACATCTGCAACGTGAACCACAAGCCCTCCAACA |
| | | | | CCAAGGTGGACAAGAAGGTGGAACCCAAGTCCTGCGAC |
| | | | | AAGACCCACACCTGTCCCCCTTGTCCTGCCCCTGAACT |
| | | | | GCTGGGCGGACCTTCCGTGTTCCTGTTCCCCCCAAAGC |
| | | | | CCAAGGACACCCTGATGATCTCCCGGACCCCCGAAGTG |
| | | | | ACCTGCGTGGTGGTGGATGTGTCCCACGAGGACCCTGA |
| | | | | AGTGAAGTTCAATTGGTACGTGGACGGCGTGGAAGTGC |
| | | | | ACAACGCCAAGACCAAGCCTAGAGAGGAACAGTACAAC |
| | | | | TCCACCTACCGGGTGGTGTCCGTGCTGACCGTGCTGCA |
| | | | | CCAGGATTGGCTGAACGGCAAAGAGTACAAGTGCAAGG |
| | | | | TGTCCAACAAGGCCCTGCCTGCCCCCATCGAAAAGACC |
| | | | | ATCTCCAAGGCCAAGGGCCAGCCCCGGGAACCCCAGGT |
| | | | | GTACACACTGCCCCCTAGCAGGGACGAGCTGACCAAGA |
| | | | | ACCAGGTGTCCCTGACCTGTCTCGTGAAAGGCTTCTAC |
| | | | | CCCTCCGATATCGCCGTGGAATGGGAGTCCAACGGCCA |
| | | | | GCCTGAGAACAACTACAAGACCACCCCCCCTGTGCTGG |
| | | | | ACTCCGACGGCTCATTCTTCCTGTACAGCAAGCTGACA |
| | | | | GTGGACAAGTCCCGGTGGCAGCAGGGCAACGTGTTCTC |
| | | | | CTGCTCCGTGATGCACGAGGCCCTGCACAACCACTACA |
| | | | | CCCAGAAGTCCCTGTCCCTGAGCCCCGGCAAGTGATGA |
| 523 | Human | IGHG | Human Heavy Chain | gcctccaccaagggcccatcggtcttccccctggcacc |
| | IgG1 | 1*03 | Constant Region | ctcctccaagagcacctctgggggcacagcggccctgg |
| | constant | | (IGHG1*03) | gctgcctggtcaaggactacttccccgaaccggtgacg |
| | region | | Nucleotide | gtgtcgtggaactcaggcgccctgaccagcggcgtgca |
| | Sequence | | | caccttcccggctgtcctacagtcctcaggactctact |
| | | | | ccctcagcagcgtggtgaccgtgccctccagcagcttg |
| | | | | ggcacccagacctacatctgcaacgtgaatcacaagcc |
| | | | | cagcaacaccaaggtggacaagagagttgagcccaaat |
| | | | | cttgtgacaaaactcacacatgcccaccgtgcccagca |
| | | | | cctgaactcctggggggaccgtcagtcttcctcttccc |
| | | | | cccaaaacccaaggacaccctcatgatctcccggaccc |
| | | | | ctgaggtcacatgcgtggtggtggacgtgagccacgaa |
| | | | | gaccctgaggtcaagttcaactggtacgtggacggcgt |
| | | | | ggaggtgcataatgccaagacaaagccgcgggaggagc |
| | | | | agtacaacagcacgtaccgtgtggtcagcgtcctcacc |
| | | | | gtcctgcaccaggactggctgaatggcaaggagtacaa |
| | | | | gtgcaaggtctccaacaaagccctcccagcccccatcg |
| | | | | agaaaaccatctccaaagccaaagggcagccccgagaa |
| | | | | ccacaggtgtacaccctgcccccatcccgggaggagat |
| | | | | gaccaagaaccaggtcagcctgacctgcctggtcaaag |
| | | | | gcttctatcccagcgacatcgccgtggagtgggagagc |
| | | | | aatgggcagccggagaacaactacaagaccacgcctcc |
| | | | | cgtgctggactccgacggctccttcttcctctatagca |
| | | | | agctcaccgtggacaagagcaggtggcagcaggggaac |
| | | | | gtcttctcatgctccgtgatgcatgaggctctgcacaa |
| | | | | ccactacacgcagaagagcctctccctgtccccgggta |
| | | | | aa |
| 524 | | | Human Heavy Chain | A S T K G P S V F P L A P S S K S T S |
| | | | Constant Region | G G T A A L G C L V K D Y F P E P V T |
| | | | (IGHG1*03) | V S W N S G A L T S G V H T F P A V L |
| | | | Protein Sequence | Q S S G L Y S L S S V V T V P S S S L |
| | | | | G T Q T Y I C N V N H K P S N T K V D |
| | | | | K R V E P K S C D K T H T C P P C P A |
| | | | | P E L L G G P S V F L F P P K P K D T |
| | | | | L M I S R T P E V T C V V V D V S H E |
| | | | | D P E V K F N W Y V D G V E V H N A K |
| | | | | T K P R E E Q Y N S T Y R V V S V L T |
| | | | | V L H Q D W L N G K E Y K C K V S N K |
| | | | | A L P A P I E K T I S K A K G Q P R E |
| | | | | P Q V Y T L P P S R E E M T K N Q V S |
| | | | | L T C L V K G F Y P S D I A V E W E S |
| | | | | N G Q P E N N Y K T T P P V L D S D G |
| | | | | S F F L Y S K L T V D K S R W Q Q G N |
| | | | | V F S C S V M H E A L H N H Y T Q K S |
| | | | | L S L S P G K |
| 525 | Human | IGHG | Human Heavy Chain | gcctccaccaagggcccatcggtcttccccctggcacc |
| | IgG1 | 1*04 | Constant Region | ctcctccaagagcacctctgggggcacagcggccctgg |
| | constant | | (IGHG1*04) | gctgcctggtcaaggactacttccccgaaccggtgacg |
| | region | | Nucleotide | gtgtcgtggaactcaggcgccctgaccagcggcgtgca |
| | | | Sequence | caccttcccggctgtcctacagtcctcaggactctact |
| | | | | ccctcagcagcgtggtgaccgtgccctccagcagcttg |
| | | | | ggcacccagacctacatctgcaacgtgaatcacaagcc |
| | | | | cagcaacaccaaggtggacaagaaagttgagcccaaat |
| | | | | cttgtgacaaaactcacacatgcccaccgtgcccagca |
| | | | | cctgaactcctggggggaccgtcagtcttcctcttccc |
| | | | | cccaaaacccaaggacaccctcatgatctcccggaccc |
| | | | | ctgaggtcacatgcgtggtggtggacgtgagccacgaa |
| | | | | gaccctgaggtcaagttcaactggtacgtggacggcgt |
| | | | | ggaggtgcataatgccaagacaaagccgcgggaggagc |
| | | | | agtacaacagcacgtaccgtgtggtcagcgtcctcacc |
| | | | | gtcctgcaccaggactggctgaatggcaaggagtacaa |
| | | | | gtgcaaggtctccaacaaagccctcccagcccccatcg |
| | | | | agaaaaccatctccaaagccaaagggcagccccgagaa |
| | | | | ccacaggtgtacaccctgcccccatcccgggatgagct |
| | | | | gaccaagaaccaggtcagcctgacctgcctggtcaaag |
| | | | | gcttctatcccagcgacatcgccgtggagtgggagagc |
| | | | | aatgggcagccggagaacaactacaagaccacgcctcc |
| | | | | cgtgctggactccgacggctccttcttcctctacagca |
| | | | | agctcaccgtggacaagagcaggtggcagcaggggaac |
| | | | | atcttctcatgctccgtgatgcatgaggctctgcacaa |
| | | | | ccactacacgcagaagagcctctccctgtctccgggta |
| | | | | aa |
| 526 | | | Human Heavy Chain | ASTKGPSVFPLAPSSKSTSGGTAALGCLVKDYFPEPVT |
| | | | Constant Region | VSWNSGALTSGVHTFPAVLQSSGLYSLSSVVTVPSSSL |
| | | | (IGHG1*04) | GTQTYICNVNHKPSNTKVDKKVEPKSCDKTHTCPPCPA |
| | | | Protein Sequence | PELLGGPSVFLFPPKPKDTLMISRTPEVTCVVVDVSHE |
| | | | | DPEVKFNWYVDGVEVHNAKTKPREEQYNSTYRVVSVLT |
| | | | | VLHQDWLNGKEYKCKVSNKALPAPIEKTISKAKGQPRE |
| | | | | PQVYTLPPSRDELTKNQVSLTCLVKGFYPSDIAVEWES |
| | | | | NGQPENNYKTTPPVLDSDGSFFLYSKLTVDKSRWQQGN |
| | | | | IFSCSVMHEALHNHYTQKSLSLSPGK |
| 527 | Human | IGHG | Human Heavy Chain | gcctccaccaagggcccatcggtcttccccctggcgcc |
| | IgG2 | 2*01 | Constant Region | ctgctccaggagcacctccgagagcacagccgccctgg |
| | constant | & | (IGHG2*01) | gctgcctggtcaaggactacttccccgaaccggtgacg |
| | region | IGHG | Nucleotide | gtgtcgtggaactcaggcgctctgaccagcggcgtgca |
| | | 2*03 | Sequence | caccttcccagctgtcctacagtcctcaggactctact |
| | | & | | ccctcagcagcgtggtgaccgtgccctccagcaacttc |
| | | IGHG | | ggcacccagacctacacctgcaacgtagatcacaagcc |
| | | 2*05 | | cagcaacaccaaggtggacaagacagttgagcgcaaat |
| | | | | gttgtgtcgagtgcccaccgtgcccagcaccacctgtg |
| | | | | gcaggaccgtcagtcttcctcttccccccaaaacccaa |
| | | | | ggacaccctcatgatctcccggacccctgaggtcacgt |
| | | | | gcgtggtggtggacgtgagccacgaagaccccgaggtc |
| | | | | cagttcaactggtacgtggacggcgtggaggtgcataa |
| | | | | tgccaagacaaagccacgggaggagcagttcaacagca |
| | | | | cgttccgtgtggtcagcgtcctcaccgttgtgcaccag |
| | | | | gactggctgaacggcaaggagtacaagtgcaaggtctc |
| | | | | caacaaaggcctcccagcccccatcgagaaaaccatct |
| | | | | ccaaaaccaaagggcagccccgagaaccacaggtgtac |
| | | | | accctgcccccatcccgggaggagatgaccaagaacca |
| | | | | ggtcagcctgacctgcctggtcaaaggcttctacccca |
| | | | | gcgacatcgccgtggagtgggagagcaatgggcagccg |
| | | | | gagaacaactacaagaccacacctcccatgctggactc |
| | | | | cgacggctccttcttcctctacagcaagctcaccgtgg |
| | | | | acaagagcaggtggcagcaggggaacgtcttctcatgc |
| | | | | tccgtgatgcatgaggctctgcacaaccactacacgca |
| | | | | gaagagcctctccctgtctccgggtaaa |
| 528 | | | Human Heavy Chain | ASTKGPSVFPLAPCSRSTSESTAALGCLVKDYFPEPVT |
| | | | Constant Region | VSWNSGALTSGVHTFPAVLQSSGLYSLSSVVTVPSSNF |
| | | | (IGHG2*01) | GTQTYTCNVDHKPSNTKVDKTVERKCCVECPPCPAPPV |
| | | | Protein Sequence | AGPSVFLFPPKPKDTLMISRTPEVTCVVVDVSHEDPEV |
| | | | | QFNWYVDGVEVHNAKTKPREEQFNSTFRVVSVLTVVHQ |
| | | | | DWLNGKEYKCKVSNKGLPAPIEKTISKTKGQPREPQVY |
| | | | | TLPPSREEMTKNQVSLTCLVKGFYPSDIAVEWESNGQP |
| | | | | ENNYKTTPPMLDSDGSFFLYSKLTVDKSRWQQGNVFSC |
| | | | | SVMHEALHNHYTQKSLSLSPGK |
| 529 | Human | IGHG | Human Heavy Chain | GCCTCCACCAAGGGCCCATCGGTCTTCCCCCTGGCGCC |
| | IgG2 | 2*02 | Constant Region | CTGCTCCAGGAGCACCTCCGAGAGCACAGCGGCCCTGG |
| | constant | | (IGHG2*02) | GCTGCCTGGTCAAGGACTACTTCCCCGAACCGGTGACG |
| | region | | Nucleotide | GTGTCGTGGAACTCAGGCGCTCTGACCAGCGGCGTGCA |
| | | | Sequence | CACCTTCCCGGCTGTCCTACAGTCCTCAGGACTCTACT |
| | | | | CCCTCAGCAGCGTGGTGACCGTGACCTCCAGCAACTTC |
| | | | | GGCACCCAGACCTACACCTGCAACGTAGATCACAAGCC |
| | | | | CAGCAACACCAAGGTGGACAAGACAGTTGAGCGCAAAT |
| | | | | GTTGTGTCGAGTGCCCACCGTGCCCAGCACCACCTGTG |
| | | | | GCAGGACCGTCAGTCTTCCTCTTCCCCCCAAAACCCAA |
| | | | | GGACACCCTCATGATCTCCCGGACCCCTGAGGTCACGT |
| | | | | GCGTGGTGGTGGACGTGAGCCACGAAGACCCCGAGGTC |
| | | | | CAGTTCAACTGGTACGTGGACGGCATGGAGGTGCATAA |
| | | | | TGCCAAGACAAAGCCACGGGAGGAGCAGTTCAACAGCA |
| | | | | CGTTCCGTGTGGTCAGCGTCCTCACCGTCGTGCACCAG |
| | | | | GACTGGCTGAACGGCAAGGAGTACAAGTGCAAGGTCTC |
| | | | | CAACAAAGGCCTCCCAGCCCCCATCGAGAAAACCATCT |
| | | | | CCAAAACCAAAGGGCAGCCCCGAGAACCACAGGTGTAC |
| | | | | ACCCTGCCCCCATCCCGGGAGGAGATGACCAAGAACCA |
| | | | | GGTCAGCCTGACCTGCCTGGTCAAAGGCTTCTACCCCA |
| | | | | GCGACATCGCCGTGGAGTGGGAGAGCAATGGGCAGCCG |
| | | | | GAGAACAACTACAAGACCACACCTCCCATGCTGGACTC |
| | | | | CGACGGCTCCTTCTTCCTCTACAGCAAGCTCACCGTGG |
| | | | | ACAAGAGCAGGTGGCAGCAGGGGAACGTCTTCTCATGC |
| | | | | TCCGTGATGCATGAGGCTCTGCACAACCACTACACACA |
| | | | | GAAGAGCCTCTCCCTGTCTCCGGGTAAA |
| 530 | | | Human Heavy Chain | ASTKGPSVFPLAPCSRSTSESTAALGCLVKDYFPEPVT |
| | | | Constant Region | VSWNSGALTSGVHTFPAVLQSSGLYSLSSVVTVTSSNF |
| | | | (IGHG2*02) | GTQTYTCNVDHKPSNTKVDKTVERKCCVECPPCPAPPV |
| | | | Protein Sequence | AGPSVFLFPPKPKDTLMISRTPEVTCVVVDVSHEDPEV |
| | | | | QFNWYVDGMEVHNAKTKPREEQFNSTFRVVSVLTVVHQ |
| | | | | DWLNGKEYKCKVSNKGLPAPIEKTISKTKGQPREPQVY |
| | | | | TLPPSREEMTKNQVSLTCLVKGFYPSDIAVEWESNGQP |
| | | | | ENNYKTTPPMLDSDGSFFLYSKLTVDKSRWQQGNVFSC |
| | | | | SVMHEALHNHYTQKSLSLSPGK |
| 531 | Human | IGHG | Human Heavy Chain | gcctccaccaagggcccatcggtcttccccctggcgcc |
| | IgG2 | 2*04 | Constant Region | ctgctccaggagcacctccgagagcacagcggccctgg |
| | constant | | (IGHG2*04) | gctgcctggtcaaggactacttccccgaaccggtgacg |
| | region | | Nucleotide | gtgtcgtggaactcaggcgctctgaccagcggcgtgca |
| | | | Sequence | caccttcccagctgtcctacagtcctcaggactctact |
| | | | | ccctcagcagcgtggtgaccgtgccctccagcagcttg |
| | | | | ggcacccagacctacacctgcaacgtagatcacaagcc |
| | | | | cagcaacaccaaggtggacaagacagttgagcgcaaat |
| | | | | gttgtgtcgagtgcccaccgtgcccagcaccacctgtg |
| | | | | gcaggaccgtcagtcttcctcttccccccaaaacccaa |
| | | | | ggacaccctcatgatctcccggacccctgaggtcacgt |
| | | | | gcgtggtggtggacgtgagccacgaagaccccgaggtc |
| | | | | cagttcaactggtacgtggacggcgtggaggtgcataa |
| | | | | tgccaagacaaagccacgggaggagcagttcaacagca |
| | | | | cgttccgtgtggtcagcgtcctcaccgttgtgcaccag |
| | | | | gactggctgaacggcaaggagtacaagtgcaaggtctc |
| | | | | caacaaaggcctcccagcccccatcgagaaaaccatct |
| | | | | ccaaaaccaaagggcagccccgagaaccacaggtgtac |
| | | | | accctgcccccatcccgggaggagatgaccaagaacca |
| | | | | ggtcagcctgacctgcctggtcaaaggcttctacccca |
| | | | | gcgacatcgccgtggagtgggagagcaatgggcagccg |
| | | | | gagaacaactacaagaccacacctcccatgctggactc |
| | | | | cgacggctccttcttcctctacagcaagctcaccgtgg |
| | | | | acaagagcaggtggcagcaggggaacgtcttctcatgc |
| | | | | tccgtgatgcatgaggctctgcacaaccactacacgca |
| | | | | gaagagcctctccctgtctccgggtaaa |
| 532 | | | Human Heavy Chain | ASTKGPSVFPLAPCSRSTSESTAALGCLVKDYFPEPVT |
| | | | Constant Region | VSWNSGALTSGVHTFPAVLQSSGLYSLSSVVTVPSSSL |
| | | | (IGHG2*04) | GTQTYTCNVDHKPSNTKVDKTVERKCCVECPPCPAPPV |
| | | | Protein Sequence | AGPSVFLFPPKPKDTLMISRTPEVTCVVVDVSHEDPEV |
| | | | | QFNWYVDGVEVHNAKTKPREEQFNSTFRVVSVLTVVHQ |
| | | | | DWLNGKEYKCKVSNKGLPAPIEKTISKTKGQPREPQVY |
| | | | | TLPPSREEMTKNQVSLTCLVKGFYPSDIAVEWESNGQP |
| | | | | ENNYKTTPPMLDSDGSFFLYSKLTVDKSRWQQGNVFSC |
| | | | | SVMHEALHNHYTQKSLSLSPGK |
| 533 | Human | IGHG | Human Heavy Chain | GCCTCCACCAAGGGCCCATCGGTCTTCCCCCTGGCGCC |
| | IgG2 | 2*06 | Constant Region | CTGCTCCAGGAGCACCTCCGAGAGCACAGCGGCCCTGG |
| | constant | | (IGHG2*06) | GCTGCCTGGTCAAGGACTACTTCCCCGAACCGGTGACG |
| | region | | Nucleotide | GTGTCGTGGAACTCAGGCGCTCTGACCAGCGGCGTGCA |
| | | | Sequence | CACCTTCCCGGCTGTCCTACAGTCCTCAGGACTCTACT |
| | | | | CCCTCAGCAGCGTGGTGACCGTGCCCTCCAGCAACTTC |
| | | | | GGCACCCAGACCTACACCTGCAACGTAGATCACAAGCC |
| | | | | CAGCAACACCAAGGTGGACAAGACAGTTGAGCGCAAAT |
| | | | | GTTGTGTCGAGTGCCCACCGTGCCCAGCACCACCTGTG |
| | | | | GCAGGACCGTCAGTCTTCCTCTTCCCCCCAAAACCCAA |
| | | | | GGACACCCTCATGATCTCCCGGACCCCTGAGGTCACGT |
| | | | | GCGTGGTGGTGGACGTGAGCCACGAAGACCCCGAGGTC |
| | | | | CAGTTCAACTGGTACGTGGACGGCGTGGAGGTGCATAA |
| | | | | TGCCAAGACAAAGCCACGGGAGGAGCAGTTCAACAGCA |
| | | | | CGTTCCGTGTGGTCAGCGTCCTCACCGTCGTGCACCAG |
| | | | | GACTGGCTGAACGGCAAGGAGTACAAGTGCAAGGTCTC |
| | | | | CAACAAAGGCCTCCCAGCCCCCATCGAGAAAACCATCT |
| | | | | CCAAAACCAAAGGGCAGCCCCGAGAACCACAGGTGTAC |
| | | | | ACCCTGCCCCCATCCCGGGAGGAGATGACCAAGAACCA |
| | | | | GGTCAGCCTGACCTGCCTGGTCAAAGGCTTCTACCCCA |
| | | | | GCGACATCTCCGTGGAGTGGGAGAGCAATGGGCAGCCG |
| | | | | GAGAACAACTACAAGACCACACCTCCCATGCTGGACTC |
| | | | | CGACGGCTCCTTCTTCCTCTACAGCAAGCTCACCGTGG |
| | | | | ACAAGAGCAGGTGGCAGCAGGGGAACGTCTTCTCATGC |
| | | | | TCCGTGATGCATGAGGCTCTGCACAACCACTACACACA |
| | | | | GAAGAGCCTCTCCCTGTCTCCGGGTAAA |
| 534 | | | Human Heavy Chain | ASTKGPSVFPLAPCSRSTSESTAALGCLVKDYFPEPVT |
| | | | Constant Region | VSWNSGALTSGVHTFPAVLQSSGLYSLSSVVTVPSSNF |
| | | | (IGHG2*06) | GTQTYTCNVDHKPSNTKVDKTVERKCCVECPPCPAPPV |
| | | | Protein Sequence | AGPSVFLFPPKPKDTLMISRTPEVTCVVVDVSHEDPEV |
| | | | | QFNWYVDGVEVHNAKTKPREEQFNSTFRVVSVLTVVHQ |
| | | | | DWLNGKEYKCKVSNKGLPAPIEKTISKTKGQPREPQVY |
| | | | | TLPPSREEMTKNQVSLTCLVKGFYPSDISVEWESNGQP |
| | | | | ENNYKTTPPMLDSDGSFFLYSKLTVDKSRWQQGNVFSC |
| | | | | SVMHEALHNHYTQKSLSLSPGK |
| 535 | Human Cλ | IGLC | Cλ Light Chain | GGTCAGCCCAAGGCTGCCCCCTCGGTCACTCTGTTCCC |
| | constant | 7*03 | Constant Region | ACCCTCCTCTGAGGAGCTTCAAGCCAACAAGGCCACAC |
| | region | | (IGLC7*03) | TGGTGTGTCTCGTAAGTGACTTCAACCCGGGAGCCGTG |
| | | | Nucleotide | ACAGTGGCCTGGAAGGCAGATGGCAGCCCCGTCAAGGT |
| | | | Sequence | GGGAGTGGAGACCACCAAACCCTCCAAACAAAGCAACA |
| | | | | ACAAGTATGCGGCCAGCAGCTACCTGAGCCTGACGCCC |
| | | | | GAGCAGTGGAAGTCCCACAGAAGCTACAGCTGCCGGGT |
| | | | | CACGCATGAAGGGAGCACCGTGGAGAAGACAGTGGCCC |
| | | | | CTGCAGAATGCTCT |
| 536 | | | Cλ Light Chain | GQPKAAPSVTLFPPSSEELQANKATLVCLVSDFNPGAV |
| | | | Constant Region | TVAWKADGSPVKVGVETTKPSKQSNNKYAASSYLSLTP |
| | | | (IGLC7*03) Amino | EQWKSHRSYSCRVTHEGSTVEKTVAPAECS |
| | | | Acid Sequence | |
| 537 | Human WT | IGHG | WT human IgG1 | gcctccaccaagggcccatcggtcttccccctggcacc |
| | IgG1 | 1*01 | nucleotide | ctcctccaagagcacctctgggggcacagcggccctgg |
| | constant | & | sequence #2 | gctgcctggtcaaggactacttccccgaaccggtgacg |
| | region | IGHG | | gtgtcgtggaactcaggcgccctgaccagcggcgtgca |
| | | 1*05 | | caccttcccggctgtcctacagtcctcaggactctact |
| | | (IgG | | ccctcagcagcgtggtgaccgtgccctccagcagcttg |
| | | 1) | | ggcacccagacctacatctgcaacgtgaatcacaagcc |
| | | | | cagcaacaccaaggtggacaagaaagttgagcccaaat |
| | | | | cttgtgacaaaactcacacatgcccaccgtgcccagca |
| | | | | cctgaactcctggggggaccgtcagtcttcctcttccc |
| | | | | cccaaaacccaaggacaccctcatgatctcccggaccc |
| | | | | ctgaggtcacatgcgtggtggtggacgtgagccacgaa |
| | | | | gaccctgaggtcaagttcaactggtacgtggacggcgt |
| | | | | ggaggtgcataatgccaagacaaagccgcgggaggagc |
| | | | | agtacaacagcacgtaccgggtggtcagcgtcctcacc |
| | | | | gtcctgcaccaggactggctgaatggcaaggagtacaa |
| | | | | gtgcaaggtctccaacaaagccctcccagcccccatcg |
| | | | | agaaaaccatctccaaagccaaagggcagccccgagaa |
| | | | | ccacaggtgtacaccctgcccccatcccgggatgagct |
| | | | | gaccaagaaccaggtcagcctgacctgcctggtcaaag |
| | | | | gcttctatcccagcgacatcgccgtggagtgggagagc |
| | | | | aatgggcagccggagaacaactacaagaccacgcctcc |
| | | | | cgtgctggactccgacggctccttcttcctctacagca |
| | | | | agctcaccgtggacaagagcaggtggcagcaggggaac |
| | | | | gtcttctcatgctccgtgatgcatgaggctctgcacaa |
| | | | | ccactacacgcagaagagcctctccctgtctccgggta |
| | | | | aa |
| 538 | Human Cλ | IGLC | Cλ Light Chain | GQPKAAPSVTLFPPSSEELQANKATLVCLISDFYPGAV |
| | constant | 2*01 | Constant Region | TVAWKADSSPVKAGVETTTPSKQSNNKYAASSYLSLTP |
| | region | | Amino Acid | EQWKSHRSYSCQVTHEGSTVEKTVAPTECS |
| | | | Sequence #2 - | |
| | | | Encoded by | |
| | | | nucleotide | |
| | | | sequence version | |
| | | | A & B | |

TABLE S3
SEQ ID NOS: 539-562

Sequence

hIgG1 FIT-Ig bispecific 1a

| | | |
|---|---|---|
| Antibody A | anti-ICOS | |
| | STIM003 | |
| Antibody B | anti-PD-L1 | |
| | 84G09 | |
| FIT-Ig | SEQ ID NO: | DIQMTQSPASLSASLGETVTIQCRASEDIYSGLAWFQQKPGKSPQLLIYGASS |
| Construct #1 | 539 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRWQQG |
| | | NVFSCSVMHEALHNHYTQKSLSLSPGK |
| FIT-Ig | SEQ ID NO: | EVQLVESGGGLVQPGRSLKLSCAASGFTFSDFYMAWVRQAPKKGLEWVASISY |
| Construct #2 | 540 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 541 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 1b

| | | |
|---|---|---|
| Antibody A | anti-PD-L1 | |
| | 84G09 | |
| Antibody B | anti-ICOS | |
| | STIM003 | |
| FIT-Ig | SEQ ID NO: | DIVMTQSPSSLAVSPGEKVTMTCKSSQSLYYSGVKENLLAWYQQKPGQSPKLL |
| Construct #1 | 542 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSKLTVDKSR |
| | | WQQGNVFSCSVMHEALHNHYTQKSLSLSPGK |
| FIT-Ig | SEQ ID NO: | EVQLVESGGGLTQPGKSLKLSCEASGFTFSSFTMHWVRQSPGKGLEWVAFIRS |
| Construct #2 | 543 | GSGIVFYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 544 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

hIgG1 FIT-Ig bispecific 2a

| | | |
|---|---|---|
| Antibody A | anti-ICOS | |
| | STIM001 | |
| Antibody B | anti-PD-L1 | |
| | 1D05 | |
| FIT-Ig | SEQ ID NO: | DIQMTQSPASLSASLGETVTIQCRASEDIYSGLAWFQQKPGKSPQLLIYGASS |
| Construct #1 | 545 | LQDGVPSRFSGSGSGTQYSLKISSMQTEDEGVYFCQQGLKYPPTFGSGTKLEI |
| | | KRTDAAPTVSIFPPSSEQLTSGGASVVCFLNNFYPKDINVKWKIDGSERQNGV |
| | | LNSWTDQDSKDSTYSMSSTLTLTKDEYERHNSYTCEATHKTSTSPIVKSFNRN |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSAKTTAPSVYPLAPVCGDTTGSSVTLGCLVKGYFPEPVT |
| | | LTWNSGSLSSGVHTFPAVLQSDLYTLSSSVTVTSSTWPSQSITCNVAHPASST |
| | | KVDKKIEPRGPTIKPCPPCKCPAPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDFMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 546 | EGSSTYYGDSVMGRFTISRDNAKSTLYLQMNSLRSEDTATYYCARQREANWED |
| | | WGQGVMVTVSSAKTTAPSVYPLAPVCGDTTGSSVTLGCLVKGYFPEPVTLTWN |
| | | SGSLSSGVHTFPAVLQSDLYTLSSSVTVTSSTWPSQSITCNVAHPASSTKVDK |
| | | KI |
| FIT-Ig | SEQ ID NO: | DIVMTQSPSSLAVSPGEKVTMTCKSSQSLYYSGVKENLLAWYQQKPGQSPKLL |
| Construct #3 | 547 | IYYASIRFTGVPDRFTGSGSGTDYTLTITSVQAEDMGQYFCQQGINNPLTFGD |
| | | GTKLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSPIV |
| | | KSFNRNEC |

hIgG1 FIT-Ig bispecific 2b

| | | |
|---|---|---|
| Antibody A | anti-PD-L1 | |
| | 1D05 | |
| Antibody B | anti-ICOS | |
| | STIM001 | |
| FIT-Ig | SEQ ID NO: | DIVMTQSPSSLAVSPGEKVTMTCKSSQSLYYSGVKENLLAWYQQKPGQSPKLL |
| Construct #1 | 548 | IYYASIRFTGVPDRFTGSGSGTDYTLTITSVQAEDMGQYFCQQGINNPLTFGD |
| | | GTKLEIKRTDAAPTVSIFPPSSEQLTSGGASVVCFLNNFYPKDINVKWKIDGS |
| | | ERQNGVLNSWTDQDSKDSTYSMSSTLTLTKDEYERHNSYTCEATHKTSTSPIV |
| | | KSFNRNECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSAKTTAPSVYPLAPVCGDTTGSSVTLGCLVKGYFP |
| | | EPVTLTWNSGSLSSGVHTFPAVLQSDLYTLSSSVTVTSSTWPSQSITCNVAHP |
| | | ASSTKVDKKIEPRGPTIKPCPPCKCPAPNLLGGPSVFIFPPKIKDVLMISLSP |
| | | IVTCVVVDVSEDDPDVQISWFVNNVEVHTAQTQTHREDYNSTLRVVSALPIQH |
| | | QDWMSGKEFKCKVNNKDLPAPIERTISKPKGSVRAPQVYVLPPPEEEMTKKQV |
| | | TLTCMVTDFMPEDIYVEWTNNGKTELNYKNTEPVLDSDGSYFMYSKLRVEKKN |
| | | WVERNSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 549 | GSGIVFYADAVRGRFTISRDNAKNLLFLQMNDLKSEDTAMYYCARRPLGHNTF |
| | | DSWGQGTLVTVSSAKTTAPSVYPLAPVCGDTTGSSVTLGCLVKGYFPEPVTLT |
| | | WNSGSLSSGVHTFPAVLQSDLYTLSSSVTVTSSTWPSQSITCNVAHPASSTKV |
| | | DKKI |
| FIT-Ig | SEQ ID NO: | DIQMTQSPASLSASLGETVTIQCRASEDIYSGLAWFQQKPGKSPQLLIYGASS |
| Construct #3 | 550 | LQDGVPSRFSGSGSGTQYSLKISSMQTEDEGVYFCQQGLKYPPTFGSGTKLEI |
| | | KRTDAAPTVSIFPPSSEQLTSGGASVVCFLNNFYPKDINVKWKIDGSERQNGV |
| | | LNSWTDQDSKDSTYSMSSTLTLTKDEYERHNSYTCEATHKTSTSPIVKSFNRN |
| | | EC |

hIgG1 FIT-Ig bispecific 3a

| | | |
|---|---|---|
| Antibody A | anti-ICOS | |
| | STIM003 | |
| Antibody B | anti-PD-L1 | |
| | 1D05 | |
| FIT-Ig | SEQ ID NO: | DIQMTQSPASLSASLGETVTIQCRASEDIYSGLAWFQQKPGKSPQLLIYGASS |
| Construct #1 | 551 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPNLLGGPSVFIFPPKIKDVLMISLSPIVTCVVVD |
| | | VSEDDPDVQISWFVNNVEVHTAQTQTHREDYNSTLRVVSALPIQHQDWMSGKE |
| | | FKCKVNNKDLPAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWVERNSYS |
| | | CSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 552 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 553 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 3b

| | | |
|---|---|---|
| Antibody A | anti-PD-L1 | |
| | 1D05 | |
| Antibody B | anti-ICOS | |
| | STIM003 | |
| FIT-Ig | SEQ ID NO: | DIVMTQSPSSLAVSPGEKVTMTCKSSQSLYYSGVKENLLAWYQQKPGQSPKLL |
| Construct #1 | 554 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDFMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 555 | GSGIVFYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 556 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

hIgG1 FIT-Ig bispecific 4a

| | | |
|---|---|---|
| Antibody A | anti-ICOS | |
| | STIM001 | |
| Antibody B | anti-PD-L1 | |
| | 84G09 | |
| FIT-Ig | SEQ ID NO: | DIQMTQSPASLSASLGETVTIQCRASEDIYSGLAWFQQKPGKSPQLLIYGASS |
| Construct #1 | 557 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | ECEVQLVESGGGLTQPGKSLKLSCEASGFTFSSFTMHWVRQSPGKGLEWVAFI |
| | | RSGSGIVFYADAVRGRFTISRDNAKNLLFLQMNDLKSEDTAMYYCARRPLGHN |
| | | TFDSWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSN |
| | | TKVDKKVEPKSCDKTHTCPPNLLGGPSVFIFPPKIKDVLMISLSPIVTCVVVD |
| | | VSEDDPDVQISWFVNNVEVHTAQTQTHREDYNSTLRVVSALPIQHQDWMSGKE |
| | | FKCKVNNKDLPAPIERTISKPKGSVRAPQVYVLPPPEEEMTKKQVTLTCMVTD |
| | | FMPEDIYVEWTNNGKTELNYKNTEPVLDSDGSYFMYSKLRVEKKNWVERNSYS |
| | | CSVVHEGLHNHHTTKSFSRTPGK |
| FIT-Ig | SEQ ID NO: | EVQLVESGGGLVQPGRSLKLSCAASGFTFSDFYMAWVRQAPKKGLEWVASISY |
| Construct #2 | 558 | EGSSTYYGDSVMGRFTISRDNAKSTLYLQMNSLRSEDTATYYCARQREANWED |
| | | WGQGVMVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKV |
| FIT-Ig | SEQ ID NO: | DIVMTQSPSSLAVSPGEKVTMTCKSSQSLYYSGVKENLLAWYQQKPGQSPKLL |
| Construct #3 | 559 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |

hIgG1 FIT-Ig bispecific 4b

| | | |
|---|---|---|
| Antibody A | anti-PD-L1 | |
| | 84G09 | |
| Antibody B | anti-ICOS | |
| | STIM001 | |
| FIT-Ig | SEQ ID NO: | DIVMTQSPSSLAVSPGEKVTMTCKSSQSLYYSGVKENLLAWYQQKPGQSPKLL |
| Construct #1 | 560 | IYYASIRFTGVPDRFTGSGSGTDYTLTITSVQAEDMGQYFCQQGINNPLTFGD |
| | | GTKLEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGECEVQLVESGGGLVQPGRSLKLSCAASGFTFSDFYMAWVRQAPKKGL |
| | | EWVASISYEGSSTYYGDSVMGRFTISRDNAKSTLYLQMNSLRSEDTATYYCAR |
| | | QREANWEDWGQGVMVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPNLLGGPSVFIFPPKIKDVLMISLSPIVTC |
| | | VVVDVSEDDPDVQISWFVNNVEVHTAQTQTHREDYNSTLRVVSALPIQHQDWM |
| | | SGKEFKCKVNNKDLPAPIERTISKPKGSVRAPQVYVLPPPEEEMTKKQVTLTC |
| | | MVTDFMPEDIYVEWTNNGKTELNYKNTEPVLDSDGSYFMYSKLRVEKKNWVER |
| | | NSYSCSVVHEGLHNHHTTKSFSRTPGK |
| FIT-Ig | SEQ ID NO: | EVQLVESGGGLTQPGKSLKLSCEASGFTFSSFTMHWVRQSPGKGLEWVAFIRS |
| Construct #2 | 561 | GSGIVFYADAVRGRFTISRDNAKNLLFLQMNDLKSEDTAMYYCARRPLGHNTF |
| | | DSWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKV |
| FIT-Ig | SEQ ID NO: | DIQMTQSPASLSASLGETVTIQCRASEDIYSGLAWFQQKPGKSPQLLIYGASS |
| Construct #3 | 562 | LQDGVPSRFSGSGSGTQYSLKISSMQTEDEGVYFCQQGLKYPPTFGSGTKLEI |
| | | KRTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNRG |
| | | EC |

### REF

1 Dong, H., et al. (1999), “PD-L1, a third member of the B7 family, co-stimulates T-cell proliferation and interleukin-10 secretion,” Nat. Med. 5 (12), 1365-1369
2 Brahmer et al., Journal of Clinical Oncology, 2010
3 Topalian et al., NEJM, 2012
4 Herbst R S, et al., “Predictive correlates of response to the anti-PD-L1 antibody MPDL3280A in cancer patients”, Nature, 2014 Nov. 27, 515(7528):563-7, doi: 10.1038/nature14011
5 Fehrenbacher et al., 2016, The Lancet, http://doi.org/10.1016/S0140-6736(16)00587-0
6 Rosenberg et al., 2016, The Lancet, http://doi.org/10.1016/S0140-6736(16)00561-4
7 Hutloff A, et al. ICOS is an inducible T-cell co-stimulator structurally and functionally related to CD28. Nature. 1999 Jan. 21; 397(6716):263-6.
8 Beier K C, et al. Induction, binding specificity and function of human ICOS. Eur J Immunol. 2000 December; 30(12):3707-17.
9 Coyle A J, et al. The CD28-related molecule ICOS is required for effective T cell-dependent immune responses. Immunity. 2000 July; 13(1):95-105.
10 Dong C, et al. ICOS co-stimulatory receptor is essential for T-cell activation and function. Nature. 2001 Jan. 4; 409(6816):97-101.
11 Mak T W, et al., Costimulation through the inducible costimulator ligand is essential for both T helper and B cell functions in T cell-dependent B cell responses. Nat Immunol. 2003 August; 4(8):765-72.
12 Swallow M M, Wallin J J, Sha W C. B7h, a novel costimulatory homolog of B7.1 and B7.2, is induced by TNFalpha. Immunity. 1999 October; 11(4):423-32.
13 Wang S, et al. Costimulation of T cells by B7-H2, a B7-like molecule that binds ICOS. Blood. 2000 Oct. 15; 96(8):2808-13.
14 Conrad C, Gilliet M. Plasmacytoid dendritic cells and regulatory T cells in the tumor microenvironment: A dangerous liaison. Oncoimmunology. 2013 May 1; 2(5):e2388.
15 Simpson et al., Fc-dependent depletion of tumor-infiltrating regulatory T cells co-defines the efficacy of anti-CTLA-4 therapy against melanoma. J. Exp. Med. 210(9):1695-1710 2013
16 Fu T, He Q, Sharma P. The ICOS/ICOSL pathway is required for optimal antitumor responses mediated by anti-CTLA-4 therapy. Cancer Res. 2011 Aug. 15; 71(16):5445-54.
17 Fan X, Quezada S A, Sepulveda M A, Sharma P, Allison J P. Engagement of the ICOS pathway markedly enhances efficacy of CTLA-4 blockade in cancer immunotherapy. J Exp Med. 2014 Apr. 7; 211(4):715-25.
18 Carthon, B. C., et al. Preoperative CTLA-4 blockade: Tolerability and immune monitoring in the setting of a presurgical clinical trial. Clin. Cancer Res. 16:2861-2871.
19 Liakou C I, et al. CTLA-4 blockade increases IFNgamma-producing CD4+ICOShi cells to shift the ratio of effector to regulatory T cells in cancer patients. Proc Natl Acad Sci USA. 2008 Sep. 30; 105(39):14987-92.
20 Vonderheide, R. H., et al. 2010. Tremelimumab in combination with exemestane in patients with advanced breast cancer and treatment-associated modulation of inducible costimulator expression on patient T cells. Clin. Cancer Res. 16:3485-3494.
21 Larkin J., et al. NEJM 373(1):23-34 2015
22 Larkin J., et al. The Oncologist 11 May 2017
23 Chattopadhyay et al., Structural Basis of Inducible Costimulatory Ligand Function: Determination of the Cell Surface Oligomeric State and Functional Mapping of the Receptor Binding Site of the Protein, J. Immunol. 177(6):3920-3929 2006

1. A composition comprising
a multispecific antibody which binds ICOS and PD-L1,
a pharmaceutically acceptable excipient, diluent or carrier, and
an anti-CTLA-4 antibody or an anti-PD-1 antibody.

2. (canceled)

3. A method of treating cancer in a human patient, comprising administering to the patient (i) a multispecific antibody which binds ICOS and PD-L1 and (ii) an anti-CTLA-4 antibody or an anti-PD-1 antibody.

4. The method of claim 3, wherein the cancer is associated with Tregs and/or tests positive for expression of ICOS and FOXP3.

5. The composition of claim 1, wherein the anti-CTLA-4 antibody is ipilimumab or tremelimumab.

6. The composition of claim 1, wherein the anti-PD-1 antibody is pembrolizumab, nivolumab or genolimzumab.

7. The composition of claim 1, wherein the multispecific antibody comprises a binding site for ICOS provided by a VH domain amino acid sequence at least 90% identical to SEQ ID NO: 408 and a VL domain amino acid sequence at least 90% identical to SEQ ID NO: 415.

8. The composition of claim 1, wherein the multispecific antibody comprises a binding site for ICOS provided by a VH domain amino acid sequence at least 95% identical to SEQ ID NO: 408.

9. The composition of claim 1, wherein the multispecific antibody comprises a binding site for ICOS provided by a VH domain having HCDR1, HCDR2 and HCDR3 sequences wherein
HCDR1 is SEQ ID NO: 405, optionally comprising a conservative substitution at residue 28;
HCDR2 is SEQ ID NO: 406, optionally comprising a substitution at residue 59, residue 63 and/or residue 64; and
HCDR3 is SEQ ID NO: 407, optionally comprising a substitution at residue 108, residue 109 and/or residue 112.

10. The composition of claim 9, wherein the substitution at residue 28 of HCDR1 is a conservative substitution, e.g., V28F.

11. The composition of claim 9,
wherein the substitution at residue 59 of HCDR2 is N59I,
wherein the substitution at residue 63 of HCDR2 is G63D, and/or
wherein the substitution at residue 64 of HCDR2 is D64N

12. The composition of claim 9,
wherein the substitution at residue 108 of HCDR3 is F108Y;
wherein the substitution at residue 109 of HCDR3 is Y109F; and/or
wherein the substitution at residue 112 of HCDR3 is H112N.

13. The composition of claim 9, comprising a VH domain having HCDR1, HCDR2 and HCDR3 sequences wherein HCDR1 is SEQ ID NO: 405, HCDR2 is SEQ ID NO: 406, and HCDR3 is SEQ ID NO: 407.

14. The composition of claim 1, wherein the antibody comprises a VH domain amino acid sequence SEQ ID NO: 408.

15. The composition of claim 1, wherein the antibody comprises a VL domain amino acid sequence at least 95% identical to SEQ ID NO: 415.

16. The composition of claim 1, wherein the multispecific antibody comprises a VL domain having LCDR1, LCDR2 and LCDR3 sequences wherein
LCDR1 is SEQ ID NO: 412, optionally comprising a substitution at residue 36,
LCDR2 is SEQ ID NO: 413, and
LCDR3 is the SEQ ID NO 414, optionally comprising a substitution at residue 108 or residue 109.

17. The composition of claim 16, wherein the substitution at residue 36 of LCDR1 is R36S.

18. The composition of claim 16, wherein the substitution at residue 108 of LCDR3 is D108G and/or wherein the substitution at residue 109 of LCDR3 is M109N.

19. (canceled)

20. The composition of claim 1, wherein the multispecific antibody comprises a VL domain amino acid sequence comprising SEQ ID NO: 415.


SEQUENCE LISTING (565 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 241 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERT

SEQ ID NO: 3 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHHHHHH

SEQ ID NO: 4 - protein, 475 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 249 aa, Macaca fascicularis
MGWSCIILFLVATATGVHSMFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTDYKDDDDK

SEQ ID NO: 6 - protein, 405 aa, Homo sapiens
MGWSCIILFLVATATGVHSLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQKLENLYFQGIEGRMDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 7 - protein, 8 aa, Homo sapiens
GFTFDDYA

SEQ ID NO: 8 - protein, 8 aa, Homo sapiens
ISWKSNII

SEQ ID NO: 9 - protein, 15 aa, Homo sapiens
ARDITGSGSYGWFDP

SEQ ID NO: 10 - protein, 5 aa, Homo sapiens
DYAMH

SEQ ID NO: 11 - protein, 17 aa, Homo sapiens
GISWKSNIIGYADSVKG

SEQ ID NO: 12 - protein, 13 aa, Homo sapiens
DITGSGSYGWFDP

SEQ ID NO: 13 - protein, 122 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSS

SEQ ID NO: 14 - DNA, 508 bp, Homo sapiens
  source 1..508: mol_type = unassigned DNA
caagaaaaagcttgccgccaccatggagtttgggctgagctggattttccttttggctattttaaaaggtgtccagtgtgaagtacaattggtggagtccgggggaggcttggtacagcctggcaggtccctgagactctcctgtgcagcctctggattcacctttgatgattatgccatgcactgggtccgacaaactccagggaagggcctggagtgggtctcaggtataagttggaagagtaatatcataggctatgcggactctgtgaagggccgattcaccatctccagagacaacgccaagaactccctgtatctgcaaatgaacagtctgagagctgaggacacggccttgtattattgtgcaagagatataacgggttcggggagttatggctggttcgacccctggggccagggaaccctggtcaccgtctcctcagccaaaacgacacccccatctgtctatccactggcccctgaatctgctaaaactcagcctccg

SEQ ID NO: 15 - protein, 449 aa, Homo sapiens
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 1356 bp, Homo sapiens
  source 1..1356: mol_type = unassigned DNA
gaagtgcagctggtggaatctggcggcggactggtgcagcctggcagatccctgagactgtcttgtgccgcctccggcttcaccttcgacgactacgctatgcactgggtgcgacagacccctggcaagggcctggaatgggtgtccggcatctcctggaagtccaacatcatcggctacgccgactccgtgaagggccggttcaccatctcccgggacaacgccaagaactccctgtacctgcagatgaacagcctgcgggccgaggacaccgccctgtactactgcgccagagacatcaccggctccggctcctacggatggttcgatccttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 17 - protein, 6 aa, Homo sapiens
QSISSY

SEQ ID NO: 18 - skipped

SEQ ID NO: 19 - protein, 9 aa, Homo sapiens
QQSYSNPIT

SEQ ID NO: 20 - protein, 11 aa, Homo sapiens
RASQSISSYLN

SEQ ID NO: 21 - protein, 7 aa, Homo sapiens
VASSLQS

SEQ ID NO: 22 - protein, 9 aa, Homo sapiens
QQSYSNPIT

SEQ ID NO: 23 - protein, 107 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIK

SEQ ID NO: 24 - DNA, 321 bp, Homo sapiens
  source 1..321: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaa

SEQ ID NO: 25 - protein, 214 aa, Homo sapiens
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, Homo sapiens
  source 1..642: mol_type = unassigned DNA
gacatccagatgacccagtctccatcctccctgtctgcatctgtaggagacagagtcaccatcacttgccgggcaagtcagagcattagcagctatttaaattggtatcagcagaaaccagggaaagcccctaagcccctgatctatgttgcatccagtttgcaaagtggggtcccatcaagtttcagtggcagtggatctgggacagatttcactctcaccatcagcagtctgcaacctgaagattttgcaacttactactgtcaacagagttacagtaatccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 27 - protein, 8 aa, Homo sapiens
GFTFDDYA

SEQ ID NO: 28 - protein, 8 aa, Homo sapiens
ISWIRTGI

SEQ ID NO: 29 - protein, 16 aa, Homo sapiens
AKDMKGSGTYGGWFDT

SEQ ID NO: 30 - protein, 5 aa, Homo sapiens
DYAMH

SEQ ID NO: 31 - protein, 17 aa, Homo sapiens
GISWIRTGIGYADSVKG

SEQ ID NO: 32 - protein, 14 aa, Homo sapiens
DMKGSGTYGGWFDT

SEQ ID NO: 33 - protein, 123 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSS

SEQ ID NO: 34 - DNA, 482 bp, Homo sapiens
  source 1..482: mol_type = unassigned DNA
aagcttgccgccaccatggagtttgggctgagctggattttccttttggctattttaaaaggtgtccagtgtgaagtgcagctggtggagtctgggggaggcttggtgcagcctggcaggtccctgagactctcctgtgcagcctctggattcacctttgatgattatgccatgcactgggtccggcaagttccagggaagggcctggaatgggtctcaggcattagttggattcgtactggcataggctatgcggactctgtgaagggccgattcaccattttcagagacaacgccaagaattccctgtatctgcaaatgaacagtctgagagctgaggacacggccttgtattactgtgcaaaagatatgaagggttcggggacttatggggggtggttcgacacctggggccagggaaccctggtcaccgtctcctcagccaaaacaacagccccatcggtctatccactggcccctgc

SEQ ID NO: 35 - protein, 450 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - DNA, 1359 bp, Homo sapiens
  source 1..1359: mol_type = unassigned DNA
gaagtgcagctggtggaatctggcggcggactggtgcagcctggcagatccctgagactgtcttgtgccgcctccggcttcaccttcgacgactacgctatgcactgggtgcgacaggtgccaggcaagggcctggaatgggtgtccggcatctcttggatccggaccggcatcggctacgccgactctgtgaagggccggttcaccatcttccgggacaacgccaagaactccctgtacctgcagatgaacagcctgcgggccgaggacaccgccctgtactactgcgccaaggacatgaagggctccggcacctacggcggatggttcgatacttggggccagggcaccctcgtgaccgtgtcctctgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 37 - protein, 6 aa, Homo sapiens
QSISSY

SEQ ID NO: 38 - skipped

SEQ ID NO: 39 - protein, 9 aa, Homo sapiens
QQSYSTPIT

SEQ ID NO: 40 - protein, 11 aa, Homo sapiens
RASQSISSYLN

SEQ ID NO: 41 - protein, 7 aa, Homo sapiens
VASSLQS

SEQ ID NO: 42 - protein, 9 aa, Homo sapiens
QQSYSTPIT

SEQ ID NO: 43 - protein, 107 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIK

SEQ ID NO: 44 - DNA, 418 bp, Homo sapiens
  source 1..418: mol_type = unassigned DNA
aaagcttgccgccaccatgaggctccctgctcagcttctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaaactcctgatctatgttgcatccagtttgcaaagtggggtcccatcaaggttcagtggcagtggatctgggacagatttcactctcactatcagcagtctgcaacctgaagattttgcaacttactactgtcaacagagttacagtaccccgatcaccttcggccaagggacacgtctggagatcaaacgtacggatgctgcaccaact

SEQ ID NO: 45 - protein, 214 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 642 bp, Homo sapiens
  source 1..642: mol_type = unassigned DNA
gacatccagatgacccagtccccctccagcctgtctgcttccgtgggcgacagagtgaccatcacctgtcgggcctcccagtccatctcctcctacctgaactggtatcagcagaagcccggcaaggcccccaagctgctgatctacgtggccagctctctgcagtccggcgtgccctctagattctccggctctggctctggcaccgactttaccctgaccatcagctccctgcagcccgaggacttcgccacctactactgccagcagtcctactccacccctatcaccttcggccagggcacccggctggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 47 - protein, 450 aa, Homo sapiens
EVQLVESGGGLVQPGRSLRLSCAASGFTFDDYAMHWVRQAPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 450 aa, Homo sapiens
EVQLVESGGGLVQPGRSLRLSCAASGFTFDDYAMHWVRQVPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 449 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, Homo sapiens
DIQMTQSPSSLSASVGDRVTITCRASQSISSYLNWYQQKPGKAPKLFIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 8 aa, Homo sapiens
GFTFSSYW

SEQ ID NO: 53 - protein, 8 aa, Homo sapiens
IKEDGSEK

SEQ ID NO: 54 - protein, 12 aa, Homo sapiens
ARNRLYSDFLDN

SEQ ID NO: 55 - protein, 5 aa, Homo sapiens
SYWMS

SEQ ID NO: 56 - protein, 17 aa, Homo sapiens
NIKEDGSEKYYVDSVKG

SEQ ID NO: 57 - protein, 10 aa, Homo sapiens
NRLYSDFLDN

SEQ ID NO: 58 - protein, 119 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSS

SEQ ID NO: 59 - DNA, 358 bp, Homo sapiens
  source 1..358: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcag

SEQ ID NO: 60 - protein, 449 aa, Homo sapiens
EVQLVESGGGLVQPGGSLRLSCAASGFTFSSYWMSWVRQAPGKGLEWVANIKEDGSEKYYVDSVKGRFTISRDNAKNSLYLQMNSLRAEDTSVYYCARNRLYSDFLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1347 bp, Homo sapiens
  source 1..1347: mol_type = unassigned DNA
gaggtgcagctggtggagtctgggggaggcttggtccagcctggggggtccctgagactctcctgtgcagcctctggattcacgtttagtagctattggatgagttgggtccgccaggctccagggaaggggctggagtgggtggccaacatcaaagaagatggaagtgagaaatactatgtcgactctgtgaagggccgattcaccatctccagagacaacgccaagaactcactgtatctgcaaatgaacagcctgagagccgaggacacgtctgtgtattactgtgcgagaaatcgactctacagtgacttccttgacaactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 62 - protein, 6 aa, Homo sapiens
QGVSSW

SEQ ID NO: 63 - skipped

SEQ ID NO: 64 - protein, 9 aa, Homo sapiens
QQANSIPFT

SEQ ID NO: 65 - protein, 11 aa, Homo sapiens
RASQGVSSWLA

SEQ ID NO: 66 - protein, 7 aa, Homo sapiens
GASSLQS

SEQ ID NO: 67 - protein, 9 aa, Homo sapiens
QQANSIPFT

SEQ ID NO: 68 - protein, 107 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIK

SEQ ID NO: 69 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaac

SEQ ID NO: 70 - protein, 214 aa, Homo sapiens
DIQMTQSPSSVSASVGDRVTITCRASQGVSSWLAWYQQKSGKAPKLLIYGASSLQSGVPSRFSGSGSGTEFILTISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagctggttagcctggtatcagcagaaatcagggaaagcccctaagctcctgatctatggtgcatccagtttgcaaagtggggtcccatcaagattcagcggcagtggatctgggacagagttcattctcaccatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 72 - protein, 8 aa, Homo sapiens
GFTFSSYW

SEQ ID NO: 73 - protein, 8 aa, Homo sapiens
IKEDGSEK

SEQ ID NO: 74 - protein, 12 aa, Homo sapiens
ARVRLYSDFLDY

SEQ ID NO: 75 - protein, 5 aa, Homo sapiens
SYWMS

SEQ ID NO: 76 - protein, 17 aa, Homo sapiens
NIKEDGSEKYYVDSLKG

SEQ ID NO: 77 - protein, 10 aa, Homo sapiens
VRLYSDFLDY

SEQ ID NO: 78 - protein, 119 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSS

SEQ ID NO: 79 - DNA, 358 bp, Homo sapiens
  source 1..358: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcag

SEQ ID NO: 80 - protein, 449 aa, Homo sapiens
EVQLVDSGGGLVQPGGSLRLSCAASGFTFSSYWMSWVRQAPGKGLEWVANIKEDGSEKYYVDSLKGRFTISRDNAKNSLYLQMNSLRAEDTSVYYCARVRLYSDFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1347 bp, Homo sapiens
  source 1..1347: mol_type = unassigned DNA
gaggtgcagctggtggactctgggggaggcttggtccagcctggggggtccctgagactctcctgtgcagcctctggattcacgtttagtagctattggatgagttgggtccgccaggctccaggaaaggggctggagtgggtggccaacataaaagaagatggaagtgagaaatactatgtagactctttgaagggccgattcaccatctccagagacaacgccaagaactcactgtatctgcaaatgaacagcctgagagccgaggacacgtctgtgtattactgtgcgagagttcgactctacagtgacttccttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 82 - protein, 6 aa, Homo sapiens
QGVSSW

SEQ ID NO: 83 - skipped

SEQ ID NO: 84 - protein, 9 aa, Homo sapiens
QQANSIPFT

SEQ ID NO: 85 - protein, 11 aa, Homo sapiens
RASQGVSSWLA

SEQ ID NO: 86 - protein, 7 aa, Homo sapiens
GASSLQS

SEQ ID NO: 87 - protein, 9 aa, Homo sapiens
QQANSIPFT

SEQ ID NO: 88 - protein, 107 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIK

SEQ ID NO: 89 - DNA, 322 bp, Homo sapiens
  source 1..322: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaac

SEQ ID NO: 90 - protein, 214 aa, Homo sapiens
DIQMTQSPSSVSASVGDRVTITCRASQGVSSWLAWYQQKSGKAPKLLIYGASSLQSGVPSRFSGSGSGTEFILSISSLQPEDFATYYCQQANSIPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 642 bp, Homo sapiens
  source 1..642: mol_type = unassigned DNA
gacatccagatgacccagtctccatcttccgtgtctgcatctgtcggagacagagtcaccatcacttgtcgggcgagtcagggtgttagcagttggttagcctggtatcagcagaaatcagggaaagcccctaagctcctgatctatggtgcctccagtttgcaaagtggggtcccatcaagattcagcggcagtggatctgggacagagttcattctcagcatcagcagcctgcagcctgaagattttgcaacttactattgtcaacaggctaacagtatcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 92 - protein, 9 aa, Homo sapiens
GGSIISSDW

SEQ ID NO: 93 - protein, 7 aa, Homo sapiens
IFHSGRT

SEQ ID NO: 94 - protein, 8 aa, Homo sapiens
ARDGSGSY

SEQ ID NO: 95 - protein, 6 aa, Homo sapiens
SSDWWN

SEQ ID NO: 96 - protein, 16 aa, Homo sapiens
EIFHSGRTNYNPSLKS

SEQ ID NO: 97 - protein, 6 aa, Homo sapiens
DGSGSY

SEQ ID NO: 98 - protein, 115 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSS

SEQ ID NO: 99 - DNA, 346 bp, Homo sapiens
  source 1..346: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcag

SEQ ID NO: 100 - protein, 445 aa, Homo sapiens
QVQLQESGPGLVKPSGTLSLTCIVSGGSIISSDWWNWVRQPPGKGLEWIGEIFHSGRTNYNPSLKSRVTISIDKSKNQFSLRLSSVTAADTAVYYCARDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1335 bp, Homo sapiens
  source 1..1335: mol_type = unassigned DNA
caggtgcagctgcaggagtcgggcccaggactggtgaagccttcggggaccctgtccctcacctgcattgtctctggtggctccatcatcagtagtgactggtggaattgggtccgccagcccccagggaaggggctggagtggattggagaaatctttcatagtgggaggaccaactacaacccgtccctcaagagtcgagtcaccatatcaatagacaagtccaagaatcagttctccctgaggctgagctctgtgaccgccgcggacacggccgtgtattactgtgcgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 102 - protein, 12 aa, Homo sapiens
QSVLYSSNNKNY

SEQ ID NO: 103 - skipped

SEQ ID NO: 104 - protein, 8 aa, Homo sapiens
QQYYSNRS

SEQ ID NO: 105 - protein, 17 aa, Homo sapiens
KSSQSVLYSSNNKNYLA

SEQ ID NO: 106 - protein, 7 aa, Homo sapiens
WASTRES

SEQ ID NO: 107 - protein, 8 aa, Homo sapiens
QQYYSNRS

SEQ ID NO: 108 - protein, 112 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIK

SEQ ID NO: 109 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaac

SEQ ID NO: 110 - protein, 219 aa, Homo sapiens
DIVMTQSPDSLAVSLGERATINCKSSQSVLYSSNNKNYLAWYQQKSGQPPKLLIYWASTRESGVPDRFSGSGSGTDFTLTISSLQTEDVAVYYCQQYYSNRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaattacttagcttggtaccagcagaaatcaggacagcctcctaagttgctcatttactgggcatctacccgggaatccggggtccctgaccgattcagtggcagcgggtctgggacagatttcactctcaccatcagcagcctgcagactgaagatgtggcagtttattactgtcagcaatattatagtaatcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 112 - protein, 8 aa, Homo sapiens
GFTFSSYW

SEQ ID NO: 113 - protein, 8 aa, Homo sapiens
IKEDGSEK

SEQ ID NO: 114 - protein, 12 aa, Homo sapiens
ARNRLYSDFLDN

SEQ ID NO: 115 - protein, 5 aa, Homo sapiens
SYWMS

SEQ ID NO: 116 - protein, 17 aa, Homo sapiens
NIKEDGSEKYYVDSVKG

SEQ ID NO: 117 - protein, 10 aa, Homo sapiens
NRLYSDFLDN

SEQ ID NO: 118 - protein, 119 aa, Homo sapiens; identical to SEQ ID NO: 58

SEQ ID NO: 119 - DNA, 358 bp, Homo sapiens; identical to SEQ ID NO: 59
  source 1..358: mol_type = unassigned DNA

SEQ ID NO: 120 - protein, 449 aa, Homo sapiens; identical to SEQ ID NO: 60

SEQ ID NO: 121 - DNA, 1347 bp, Homo sapiens; identical to SEQ ID NO: 61
  source 1..1347: mol_type = unassigned DNA

SEQ ID NO: 122 - protein, 6 aa, Homo sapiens
QGVSSW

SEQ ID NO: 123 - skipped

SEQ ID NO: 124 - protein, 9 aa, Homo sapiens
QQANSIPFT

SEQ ID NO: 125 - protein, 11 aa, Homo sapiens
RASQGVSSWLA

SEQ ID NO: 126 - protein, 7 aa, Homo sapiens
GASSLQS

SEQ ID NO: 127 - protein, 9 aa, Homo sapiens
QQANSIPFT

SEQ ID NO: 128 - protein, 107 aa, Homo sapiens; identical to SEQ ID NO: 68

SEQ ID NO: 129 - DNA, 322 bp, Homo sapiens; identical to SEQ ID NO: 69
  source 1..322: mol_type = unassigned DNA

SEQ ID NO: 130 - protein, 214 aa, Homo sapiens; identical to SEQ ID NO: 70

SEQ ID NO: 131 - DNA, 642 bp, Homo sapiens; identical to SEQ ID NO: 71
  source 1..642: mol_type = unassigned DNA

SEQ ID NO: 132 - protein, 8 aa, Homo sapiens
GFTFRIYG

SEQ ID NO: 133 - protein, 8 aa, Homo sapiens
IWYDGSNK

SEQ ID NO: 134 - protein, 11 aa, Homo sapiens
ARDMDYFGMDV

SEQ ID NO: 135 - protein, 5 aa, Homo sapiens
IYGMH

SEQ ID NO: 136 - protein, 17 aa, Homo sapiens
VIWYDGSNKYYADSVKG

SEQ ID NO: 137 - protein, 9 aa, Homo sapiens
DMDYFGMDV

SEQ ID NO: 138 - protein, 118 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSS

SEQ ID NO: 139 - DNA, 355 bp, Homo sapiens
  source 1..355: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcag

SEQ ID NO: 140 - protein, 448 aa, Homo sapiens
QVQLVESGGGVVQPGRSLRLSCAASGFTFRIYGMHWVRQAPGKGLEWVAVIWYDGSNKYYADSVKGRFTISRDNSDNTLYLQMNSLRAEDTAVYYCARDMDYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1344 bp, Homo sapiens
  source 1..1344: mol_type = unassigned DNA
caggtgcagctggtggagtctgggggaggcgtggtccagcctgggaggtccctgagactctcctgtgcagcgtctggattcaccttccgtatttatggcatgcactgggtccgccaggctccaggcaaggggctggagtgggtggcagttatatggtatgatggaagtaataaatactatgctgactccgtgaagggccgattcaccatctccagagacaattccgacaacacgctgtatctgcaaatgaacagcctgagagccgaggacacggctgtgtattactgtgcgagagatatggactacttcggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 142 - protein, 6 aa, Homo sapiens
QGIRND

SEQ ID NO: 143 - skipped

SEQ ID NO: 144 - protein, 9 aa, Homo sapiens
LQHNSYPRT

SEQ ID NO: 145 - protein, 11 aa, Homo sapiens
RASQGIRNDLG

SEQ ID NO: 146 - protein, 7 aa, Homo sapiens
AASSLQS

SEQ ID NO: 147 - protein, 9 aa, Homo sapiens
LQHNSYPRT

SEQ ID NO: 148 - protein, 107 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 149 - DNA, 322 bp, Homo sapiens
  source 1..322: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaac

SEQ ID NO: 150 - protein, 214 aa, Homo sapiens
DLQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, Homo sapiens
  source 1..642: mol_type = unassigned DNA
gacctccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 152 - protein, 9 aa, Homo sapiens
GGSISSSDW

SEQ ID NO: 153 - protein, 7 aa, Homo sapiens
IFHSGNT

SEQ ID NO: 154 - protein, 8 aa, Homo sapiens
VRDGSGSY

SEQ ID NO: 155 - protein, 6 aa, Homo sapiens
SSDWWS

SEQ ID NO: 156 - protein, 16 aa, Homo sapiens
EIFHSGNTNYNPSLKS

SEQ ID NO: 157 - protein, 6 aa, Homo sapiens
DGSGSY

SEQ ID NO: 158 - protein, 115 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSS

SEQ ID NO: 159 - DNA, 346 bp, Homo sapiens
  source 1..346: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcag

SEQ ID NO: 160 - protein, 445 aa, Homo sapiens
QVQLQESGPGLVKPSGTLSLTCAVSGGSISSSDWWSWVRQPPGKGLEWIGEIFHSGNTNYNPSLKSRVTISVDKSKNQISLRLNSVTAADTAVYYCVRDGSGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 1335 bp, Homo sapiens
  source 1..1335: mol_type = unassigned DNA
caggtgcagctgcaggagtcgggcccaggactggtgaagccttcggggaccctgtccctcacctgcgctgtctctggtggctccatcagcagtagtgactggtggagttgggtccgccagcccccagggaaggggctggagtggattggggaaatctttcatagtgggaacaccaactacaacccgtccctcaagagtcgagtcaccatatcagtagacaagtccaagaaccagatctccctgaggctgaactctgtgaccgccgcggacacggccgtgtattactgtgtgagagatggttcggggagttactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 162 - protein, 12 aa, Homo sapiens
QSVLYSSNNKNY

SEQ ID NO: 163 - skipped

SEQ ID NO: 164 - protein, 8 aa, Homo sapiens
QQYYSTRS

SEQ ID NO: 165 - protein, 17 aa, Homo sapiens
KSSQSVLYSSNNKNYLA

SEQ ID NO: 166 - protein, 7 aa, Homo sapiens
WASTRES

SEQ ID NO: 167 - protein, 8 aa, Homo sapiens
QQYYSTRS

SEQ ID NO: 168 - protein, 112 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIK

SEQ ID NO: 169 - DNA, 337 bp, Homo sapiens
  source 1..337: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaac

SEQ ID NO: 170 - protein, 219 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTRSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - DNA, 657 bp, Homo sapiens
  source 1..657: mol_type = unassigned DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtactcgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 172 - protein, 10 aa, Homo sapiens
GGSISSSSYY

SEQ ID NO: 173 - protein, 7 aa, Homo sapiens
IYSTGYT

SEQ ID NO: 174 - protein, 13 aa, Homo sapiens
AISTAAGPEYFHR

SEQ ID NO: 175 - protein, 7 aa, Homo sapiens
SSSYYCG

SEQ ID NO: 176 - protein, 16 aa, Homo sapiens
SIYSTGYTYYNPSLKS

SEQ ID NO: 177 - protein, 11 aa, Homo sapiens
STAAGPEYFHR

SEQ ID NO: 178 - protein, 120 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSS

SEQ ID NO: 179 - DNA, 361 bp, Homo sapiens
  source 1..361: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcag

SEQ ID NO: 180 - protein, 450 aa, Homo sapiens
QLQESGPGLVKPSETLSLTCTVSGGSISSSSYYCGWIRQPPGKGLDWIGSIYSTGYTYYNPSLKSRVTISIDTSKNQFSCLILTSVTAADTAVYYCAISTAAGPEYFHRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1350 bp, Homo sapiens
  source 1..1350: mol_type = unassigned DNA
cagctgcaggagtcgggcccaggcctggtgaagccttcggagaccctgtccctcacctgcactgtctctggtggctccatcagcagtagtagttattactgcggctggatccgccagccccctgggaaggggctggactggattgggagtatctattctactgggtacacctactacaacccgtccctcaagagtcgagtcaccatttccatagacacgtccaagaaccagttctcatgcctgatactgacctctgtgaccgccgcagacacggctgtgtattactgtgcgataagtacagcagctggccctgaatacttccatcgctggggccagggcaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 182 - protein, 12 aa, Homo sapiens
QSVLYSSNSKNF

SEQ ID NO: 183 - skipped

SEQ ID NO: 184 - protein, 9 aa, Homo sapiens
QQYYSTPRT

SEQ ID NO: 185 - protein, 17 aa, Homo sapiens
KSSQSVLYSSNSKNFLA

SEQ ID NO: 186 - protein, 7 aa, Homo sapiens
WASTRGS

SEQ ID NO: 187 - protein, 9 aa, Homo sapiens
QQYYSTPRT

SEQ ID NO: 188 - protein, 113 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIK

SEQ ID NO: 189 - DNA, 340 bp, Homo sapiens
  source 1..340: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaac

SEQ ID NO: 190 - protein, 220 aa, Homo sapiens
DIVMTQSPDSLAVSLGERATINCKSSQSVLYSSNSKNFLAWYQQKPGQPPKLFIYWASTRGSGVPDRISGSGSGTDFNLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 660 bp, Homo sapiens
  source 1..660: mol_type = unassigned DNA
gacatcgtgatgacccagtctccagactccctggctgtgtctctgggcgagagggccaccatcaactgcaagtccagccagagtgttttatacagctccaacagtaagaacttcttagcttggtaccagcagaaaccgggacagcctcctaagctgttcatttactgggcatctacccggggatccggggtccctgaccgaatcagtggcagcgggtctgggacagatttcaatctcaccatcagcagcctgcaggctgaagatgtggcagtttattactgtcaacaatattatagtactcctcggacgttcggccaagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 192 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 193 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 195 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - DNA, 981 bp, Homo sapiens
  source 1..981: mol_type = unassigned DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 197 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 198 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 199 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - DNA, 981 bp, Homo sapiens
  source 1..981: mol_type = unassigned DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 201 - DNA, 981 bp, Homo sapiens
  source 1..981: mol_type = unassigned DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 202 - DNA, 981 bp, Homo sapiens
  source 1..981: mol_type = unassigned DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 203 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 205 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - DNA, 321 bp, Homo sapiens
  source 1..321: mol_type = unassigned DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 207 - protein, 107 aa, Homo sapiens [sequence omitted]

SEQ ID NO: 208 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 209 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 210 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 211 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 212 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 213 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 214 - DNA, 321 bp, Homo sapiens
  source 1..321: mol_type = unassigned DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 215 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 216 - DNA, 312 bp, Homo sapiens
  source 1..312: mol_type = unassigned DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 217 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 218 - DNA, 318 bp, Homo sapiens
  source 1..318: mol_type = unassigned DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 219 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 220 - DNA, 318 bp, Homo sapiens; identical to SEQ ID NO: 218
  source 1..318: mol_type = unassigned DNA

SEQ ID NO: 221 - DNA, 318 bp, Homo sapiens
  source 1..318: mol_type = unassigned DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 222 - DNA, 318 bp, Homo sapiens
  source 1..318: mol_type = unassigned DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 223 - protein, 106 aa, Homo sapiens; identical to SEQ ID NO: 219

SEQ ID NO: 224 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 225 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 226 - DNA, 312 bp, Homo sapiens
  source 1..312: mol_type = unassigned DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 227 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 228 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 229 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 230 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 231 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 232 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 233 - protein, 106 aa, Homo sapiens; identical to SEQ ID NO: 225

SEQ ID NO: 234 - DNA, 318 bp, Homo sapiens
  source 1..318: mol_type = unassigned DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 235 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 236 - DNA, 318 bp, Homo sapiens
  source 1..318: mol_type = unassigned DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 237 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 238 - protein, 8 aa, Homo sapiens
GFTFSDYY

SEQ ID NO: 239 - protein, 8 aa, Homo sapiens
ISTSGSTI

SEQ ID NO: 240 - protein, 16 aa, Homo sapiens
ARGITGTNFYHYGLGV

SEQ ID NO: 241 - protein, 5 aa, Homo sapiens
DYYMS

SEQ ID NO: 242 - protein, 17 aa, Homo sapiens
YISTSGSTIYYADSVKG

SEQ ID NO: 243 - protein, 14 aa, Homo sapiens
GITGTNFYHYGLGV

SEQ ID NO: 244 - protein, 123 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSS

SEQ ID NO: 245 - DNA, 370 bp, Homo sapiens
  source 1..370: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcag

SEQ ID NO: 246 - protein, 453 aa, Homo sapiens
QVQLVESGGGLVKPGGSLRLSCAASGFTFSDYYMSWIRQVPGKGLEWVSYISTSGSTIYYADSVKGRFTISRDNAKNSLYLQMNSLRAEDAAVYHCARGITGTNFYHYGLGVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1359 bp, Homo sapiens
  source 1..1359: mol_type = unassigned DNA
caggtgcagctggtggagtctgggggaggcttggtcaagcctggagggtccctgagactctcctgtgcagcctctggattcaccttcagtgactactacatgagctggatccgccaggttccagggaaggggctggagtgggtttcatacattagtactagtggtagtaccatatactacgcagactctgtgaagggccgattcaccatctccagggacaacgccaagaactcactgtatctacaaatgaacagcctgagagccgaggacgcggccgtgtatcactgtgcgagaggtataactggaactaacttctaccactacggtttgggcgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 248 - protein, 6 aa, Homo sapiens
QGINSW

SEQ ID NO: 249 - skipped

SEQ ID NO: 250 - protein, 9 aa, Homo sapiens
QQVNSFPLT

SEQ ID NO: 251 - protein, 11 aa, Homo sapiens
RASQGINSWLA

SEQ ID NO: 252 - protein, 7 aa, Homo sapiens
AASTLQS

SEQ ID NO: 253 - protein, 9 aa, Homo sapiens
QQVNSFPLT

SEQ ID NO: 254 - protein, 107 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIK

SEQ ID NO: 255 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaac

SEQ ID NO: 256 - protein, 214 aa, Homo sapiens
DIQMTQSPSSVSASVGDRVTITCRASQGINSWLAWYQQKPGKAPKLLIYAASTLQSGVPSRFSGSGSGADFTLTISSLQPEDFATYYCQQVNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaacagctggttagcctggtatcagcagaaaccagggaaagcccctaagctcctgatctatgctgcatccactttgcaaagtggggtcccatcaaggttcagcggcagtgggtctggggcagatttcactctcaccatcagcagcctgcagcctgaagattttgcaacttactattgtcaacaggttaacagtttcccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 258 - protein, 8 aa, Homo sapiens
GFTFSYYA

SEQ ID NO: 259 - protein, 8 aa, Homo sapiens
ISGGGGNT

SEQ ID NO: 260 - protein, 16 aa, Homo sapiens
AKDRMKQLVRAYYFDY

SEQ ID NO: 261 - protein, 5 aa, Homo sapiens
YYAMS

SEQ ID NO: 262 - protein, 17 aa, Homo sapiens
TISGGGGNTHYADSVKG

SEQ ID NO: 263 - protein, 14 aa, Homo sapiens
DRMKQLVRAYYFDY

SEQ ID NO: 264 - protein, 123 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSS

SEQ ID NO: 265 - DNA, 370 bp, Homo sapiens
  source 1..370: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcag

SEQ ID NO: 266 - protein, 453 aa, Homo sapiens
EVPLVESGGGLVQPGGSLRLSCAASGFTFSYYAMSWVRQAPGKGLDWVSTISGGGGNTHYADSVKGRFTISRDNSKNTLYLHMNSLRAEDTAVYYCAKDRMKQLVRAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - DNA, 1359 bp, Homo sapiens
  source 1..1359: mol_type = unassigned DNA
gaggtgccgctggtggagtctgggggaggcttggtacagcctggggggtccctgagactctcctgtgcagcctctggattcacgtttagctactatgccatgagctgggtccgtcaggctccagggaaggggctggactgggtctcaactattagtggtggtggtggtaacacacactacgcagactccgtgaagggccgattcactatatccagagacaattccaagaacacgctgtatctgcacatgaacagcctgagagccgaagacacggccgtctattactgtgcgaaggatcggatgaaacagctcgtccgggcctactactttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 268 - protein, 6 aa, Homo sapiens
QDISTY

SEQ ID NO: 269 - skipped

SEQ ID NO: 270 - protein, 9 aa, Homo sapiens
QQLHTDPIT

SEQ ID NO: 271 - protein, 11 aa, Homo sapiens
WASQDISTYLG

SEQ ID NO: 272 - protein, 7 aa, Homo sapiens
GTSSLQS

SEQ ID NO: 273 - protein, 9 aa, Homo sapiens
QQLHTDPIT

SEQ ID NO: 274 - protein, 107 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIK

SEQ ID NO: 275 - DNA, 322 bp, Homo sapiens
  source 1..322: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaac

SEQ ID NO: 276 - protein, 214 aa, Homo sapiens
DIQLTQSPSFLSASVGDRVTITCWASQDISTYLGWYQQKPGKAPKLLIYGTSSLQSGVPSRFSGSGSGTEFTLTISSLQPEDFATYYCQQLHTDPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - DNA, 642 bp, Homo sapiens
  source 1..642: mol_type = unassigned DNA
gacatccagttgacccagtctccatccttcctgtctgcatctgtaggagacagagtcaccatcacttgctgggccagtcaggacattagcacttatttaggctggtatcagcaaaaaccagggaaagcccctaagctcctgatctatggtacatccagtttgcaaagtggggtcccatcaaggttcagcggcagtggatctgggacagaattcactctcacaatcagcagcctgcagcctgaagattttgcaacttattactgtcaacagcttcatactgacccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 278 - protein, 8 aa, Homo sapiens
GFTFSSYW

SEQ ID NO: 279 - protein, 8 aa, Homo sapiens
IKQDGSEK

SEQ ID NO: 280 - protein, 12 aa, Homo sapiens
ARVRQWSDYSDY

SEQ ID NO: 281 - protein, 5 aa, Homo sapiens
SYWMN

SEQ ID NO: 282 - protein, 17 aa, Homo sapiens
NIKQDGSEKYYVDSVKG

SEQ ID NO: 283 - protein, 10 aa, Homo sapiens
VRQWSDYSDY

SEQ ID NO: 284 - protein, 119 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSS

SEQ ID NO: 285 - DNA, 358 bp, Homo sapiens
  source 1..358: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcag

SEQ ID NO: 286 - protein, 449 aa, Homo sapiens
EVHLVESGGGLVQPGGSLRLSCAASGFTFSSYWMNWVRQAPGKGLEWVANIKQDGSEKYYVDSVKGRFTVSRDNAKNSLYLQMNSLRAEDTAVYYCARVRQWSDYSDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - DNA, 1347 bp, Homo sapiens
  source 1..1347: mol_type = unassigned DNA
gaggtgcacctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgcttcaccgtctccagagacaacgccaagaactcactgtatctgcaaatgaacagcctgagagccgaggacacggctgtgtattactgtgcgagagttcgacaatggtccgactactctgactactggggccagggaaccccggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaag

SEQ ID NO: 288 - protein, 6 aa, Homo sapiens
QGISSW

SEQ ID NO: 289 - skipped

SEQ ID NO: 290 - protein, 9 aa, Homo sapiens
QQANSFPFT

SEQ ID NO: 291 - protein, 11 aa, Homo sapiens
RASQGISSWLA

SEQ ID NO: 292 - protein, 7 aa, Homo sapiens
AASSLQS

SEQ ID NO: 293 - protein, 9 aa, Homo sapiens
QQANSFPFT

SEQ ID NO: 294 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIK

SEQ ID NO: 295 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaac

SEQ ID NO: 296 - protein, 214 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 298 - protein, 214 aa, Homo sapiens
DIQMTQSPSSLSASVGDRVTITCRASQSISSYLNWYQQKPGKAPKLLIYYASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 453 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 347 aa, Homo sapiens
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 301 - protein, 133 aa, Homo sapiens [sequence omitted]

SEQ ID NO: 302 - protein, 586 aa, Homo sapiens
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 303 - protein, 14 aa, Homo sapiens
APTSTQLQLELLLD

SEQ ID NO: 304 - protein, 11 aa, Homo sapiens
TQLQLEHLLLD

SEQ ID NO: 305 - protein, 17 aa, Homo sapiens
APTSKKTQLQLEHLLLD

SEQ ID NO: 306 - protein, 19 aa, Homo sapiens
PTSSSTKKTQLQLEHLLLD

SEQ ID NO: 307 - protein, 18 aa, Homo sapiens
TSSSTKKTQLQLEHLLLD

SEQ ID NO: 308 - protein, 17 aa, Homo sapiens
SSSTKKTQLQLEHLLLD

SEQ ID NO: 309 - protein, 16 aa, Homo sapiens
SSTKKTQLQLEHLLLD

SEQ ID NO: 310 - protein, 15 aa, Homo sapiens
STKKTQLQLEHLLLD

SEQ ID NO: 311 - protein, 14 aa, Homo sapiens
TKKTQLQLEHLLLD

SEQ ID NO: 312 - protein, 13 aa, Homo sapiens
KKTQLQLEHLLLD

SEQ ID NO: 313 - protein, 12 aa, Homo sapiens
KTQLQLEHLLLD

SEQ ID NO: 314 - protein, 19 aa, Homo sapiens
APTSSSTKTQLQLEHLLLD

SEQ ID NO: 315 - protein, 18 aa, Homo sapiens
APTSSSTTQLQLEHLLLD

SEQ ID NO: 316 - protein, 17 aa, Homo sapiens
APTSSSTQLQLEHLLLD

SEQ ID NO: 317 - protein, 16 aa, Homo sapiens
APTSSTQLQLEHLLLD

SEQ ID NO: 318 - protein, 14 aa, Homo sapiens
APTTQLQLEHLLLD

SEQ ID NO: 319 - protein, 13 aa, Homo sapiens
APTQLQLEHLLLD

SEQ ID NO: 320 - protein, 12 aa, Homo sapiens
ATQLQLEHLLLD

SEQ ID NO: 321 - protein, 15 aa, Homo sapiens
ATKKTQLQLEHLLLD

SEQ ID NO: 322 - protein, 15 aa, Homo sapiens
APKKTQLQLEHLLLD

SEQ ID NO: 323 - protein, 15 aa, Homo sapiens
APTKTQLQLEHLLLD

SEQ ID NO: 324 - protein, 113 aa, Homo sapiens
LQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 325 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 326 - protein, 226 aa, Mus musculus
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHHHHHH

SEQ ID NO: 327 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 328 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 329 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 330 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 331 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 332 - protein, 133 aa, Homo sapiens [sequence omitted]

SEQ ID NO: 333 - protein, 127 aa, Homo sapiens [sequence omitted]

SEQ ID NO: 334 - protein, 171 aa, Homo sapiens
CDLPQNHGLLSRNTLVLLHQMRRISPFLCLKDRRDFRFPQEMVKGSQLQKAHVMSVLHEMLQQIFSLFHTERSSAAWNMTLLDQLHTELHQQLQHLETCLLQVVGEGESAGAISSPALTLRRYFQGIRVYLKEKKYSDCAWEVVRMEIMKSLFLSTNMQERLRSKDRDLGS

SEQ ID NO: 335 - protein, 177 aa, Homo sapiens
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 336 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 337 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 338 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 339 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 340 - protein, 330 aa, Homo sapiens [sequence omitted]

SEQ ID NO: 341 - DNA, 996 bp, Homo sapiens
  source 1..996: mol_type = unassigned DNA
gccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 342 - protein, 450 aa, Homo sapiens
EVQLVESGGGLVQPGRSLRLSCAASGFTFDDYAMHWVRQAPGKGLEWVSGISWIRTGIGYADSVKGRFTISRDNAKNSLYLQMNSLRAEDTALYYCAKDMKGSGTYGGWFDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPELAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 343 - protein, 8 aa, Homo sapiens
GFTFSNYA

SEQ ID NO: 344 - protein, 8 aa, Homo sapiens
ISFSGGTT

SEQ ID NO: 345 - protein, 14 aa, Homo sapiens
AKDEAPAGATFFDS

SEQ ID NO: 346 - protein, 5 aa, Homo sapiens
NYAMS

SEQ ID NO: 347 - protein, 17 aa, Homo sapiens
AISFSGGTTYYADSVKG

SEQ ID NO: 348 - protein, 12 aa, Homo sapiens
DEAPAGATFFDS

SEQ ID NO: 349 - protein, 121 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSS

SEQ ID NO: 350 - DNA, 364 bp, Homo sapiens
  source 1..364: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcag

SEQ ID NO: 351 - protein, 448 aa, Homo sapiens
EVQLAESGGGLVQPGGSLRLSCAASGFTFSNYAMSWVRQTPGKGLEWVSAISFSGGTTYYADSVKGRFTISRDNSKNTLYLHMNSLRADDTAVYYCAKDEAPAGATFFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 352 - DNA, 1344 bp, Homo sapiens
  source 1..1344: mol_type = unassigned DNA
gaagtgcaactggcggagtctgggggaggcttggtacagccgggggggtccctgagactctcctgtgcagcctctggattcacctttagcaactatgccatgagttgggtccgccagactccaggaaaggggctggagtgggtctcagctattagttttagtggtggtactacatactacgctgactccgtgaagggccggttcaccatctccagagacaattccaagaacacgctgtatttgcacatgaacagcctgagagccgatgacacggccgtatattactgtgcgaaagatgaggcaccagctggcgcaaccttctttgactcctggggccagggaacgctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 353 - protein, 6 aa, Homo sapiens
QGIRRW

SEQ ID NO: 354 - skipped

SEQ ID NO: 355 - protein, 9 aa, Homo sapiens
QQANSFPIT

SEQ ID NO: 356 - protein, 11 aa, Homo sapiens
RASQGIRRWLA

SEQ ID NO: 357 - protein, 7 aa, Homo sapiens
GASSLQS

SEQ ID NO: 358 - protein, 9 aa, Homo sapiens
QQANSFPIT

SEQ ID NO: 359 - protein, 107 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIK

SEQ ID NO: 360 - DNA, 322 bp, Homo sapiens
  source 1..322: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaac

SEQ ID NO: 361 - protein, 214 aa, Homo sapiens
DIQMTQSPSSVSASVGDRVTITCRASQGIRRWLAWYQQKPGKAPKLLISGASSLQSGVPSRFSGSGSGTDFTLIITSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - DNA, 642 bp, Homo sapiens
  source 1..642: mol_type = unassigned DNA
gacatccagatgacccagtctccatcttccgtgtctgcatctgtaggagacagagtcaccatcacttgtcgggcgagtcagggtattaggaggtggttagcctggtatcagcagaaaccagggaaagcccctaaactcctgatctctggtgcatccagtttgcaaagtggggtcccatcaaggttcagcggcagtggatctgggacagatttcactctcatcattaccagtctgcagcctgaagattttgcaacttactattgtcaacaggctaacagtttcccgatcaccttcggccaagggacacgactggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 363 - protein, 8 aa, Homo sapiens
GYTFSTFG

SEQ ID NO: 364 - protein, 8 aa, Homo sapiens
ISAYNGDT

SEQ ID NO: 365 - protein, 14 aa, Homo sapiens
ARSSGHYYYYGMDV

SEQ ID NO: 366 - protein, 121 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSS

SEQ ID NO: 367 - DNA, 363 bp, Homo sapiens
  source 1..363: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctca

SEQ ID NO: 368 - protein, 451 aa, Homo sapiens
QVQVVQSGAEVKKPGASVKVSCKASGYTFSTFGITWVRQAPGQGLEWMGWISAYNGDTNYAQNLQGRVIMTTDTSTSTAYMELRSLRSDDTAVYYCARSSGHYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - DNA, 1359 bp, Homo sapiens
  source 1..1359: mol_type = unassigned DNA
caggttcaggtggtgcagtctggagctgaggtgaagaagcctggggcctcagtgaaggtctcctgcaaggcttctggttacaccttttccacctttggtatcacctgggtgcgacaggcccctggacaagggcttgaatggatgggatggatcagcgcttacaatggtgacacaaactatgcacagaatctccagggcagagtcatcatgaccacagacacatccacgagcacagcctacatggagctgaggagcctgagatctgacgacacggccgtttattactgtgcgaggagcagtggccactactactactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 370 - protein, 11 aa, Homo sapiens
QSLLHSNEYNY

SEQ ID NO: 371 - skipped

SEQ ID NO: 372 - protein, 9 aa, Homo sapiens
MQSLQTPLT

SEQ ID NO: 373 - protein, 112 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIK

SEQ ID NO: 374 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaa

SEQ ID NO: 375 - protein, 219 aa, Homo sapiens
DIVMTQSPLSLPVTPGEPASISCRSSQSLLHSNEYNYLDWYLQKPGQSPQLLIFLGSNRASGVPDRFSGSGSGTDFTLKITRVEAEDVGIYYCMQSLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatgaatacaactatttggattggtacctgcagaagccagggcagtctccacagctcctgatctttttgggttctaatcgggcctccggggtccctgacaggttcagtggcagtggatcaggcacagattttacactgaaaatcaccagagtggaggctgaggatgttggaatttattactgcatgcaatctctacaaactccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 377 - protein, 8 aa, Homo sapiens
GYTFTSYG

SEQ ID NO: 378 - protein, 8 aa, Homo sapiens
ISAYNGNT

SEQ ID NO: 379 - protein, 17 aa, Homo sapiens
ARSTYFYGSGTLYGMDV

SEQ ID NO: 380 - protein, 124 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSS

SEQ ID NO: 381 - DNA, 372 bp, Homo sapiens
  source 1..372: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctca

SEQ ID NO: 382 - protein, 454 aa, Homo sapiens
QVQLVQSGGEVKKPGASVKVSCKASGYTFTSYGFSWVRQAPGQGLEWMGWISAYNGNTNYAQKLQGRVTMTTDTSTSTAYMELRSLRSDDTAVYYCARSTYFYGSGTLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - DNA, 1368 bp, Homo sapiens
  source 1..1368: mol_type = unassigned DNA
caggttcaactggtgcagtctggaggtgaggtgaagaagcctggggcctcagtgaaggtctcctgcaaggcttctggttacacctttaccagctatggtttcagctgggtgcgacaggcccctggacaaggactagagtggatgggatggatcagcgcttacaatggtaacacaaactatgcacagaagctccagggcagagtcaccatgaccacagacacatccacgagcacagcctacatggagctgaggagcttgagatctgacgacacggccgtgtattactgtgcgagatctacgtatttctatggttcggggaccctctacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 384 - protein, 11 aa, Homo sapiens
QSLLHSDGYNY

SEQ ID NO: 385 - skipped

SEQ ID NO: 386 - protein, 9 aa, Homo sapiens
MQALQTPLS

SEQ ID NO: 387 - protein, 112 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIK

SEQ ID NO: 388 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaa

SEQ ID NO: 389 - protein, 219 aa, Homo sapiens
DIVMTQSPLSLPVTPGEPASISCRSSQSLLHSDGYNYLDWYLQKPGQSPQLLIYLGSTRASGFPDRFSGSGSGTDFTLKISRVEAEDVGVYYCMQALQTPLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 390 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactgtttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgtgcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 391 - protein, 8 aa, Homo sapiens
GYTFTSYG

SEQ ID NO: 392 - protein, 8 aa, Homo sapiens
ISAYNGNT

SEQ ID NO: 393 - protein, 17 aa, Homo sapiens
ARSTYFYGSGTLYGMDV

SEQ ID NO: 394 - protein, 124 aa, Homo sapiens; identical to SEQ ID NO: 380

SEQ ID NO: 395 - DNA, 372 bp, Homo sapiens; identical to SEQ ID NO: 381
  source 1..372: mol_type = unassigned DNA

SEQ ID NO: 396 - protein, 454 aa, Homo sapiens; identical to SEQ ID NO: 382

SEQ ID NO: 397 - DNA, 1368 bp, Homo sapiens; identical to SEQ ID NO: 383
  source 1..1368: mol_type = unassigned DNA

SEQ ID NO: 398 - protein, 11 aa, Homo sapiens
QSLLHSDGYNC

SEQ ID NO: 399 - skipped

SEQ ID NO: 400 - protein, 9 aa, Homo sapiens
MQALQTPCS

SEQ ID NO: 401 - protein, 112 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIK

SEQ ID NO: 402 - DNA, 336 bp, Homo sapiens; identical to SEQ ID NO: 388
  source 1..336: mol_type = unassigned DNA

SEQ ID NO: 403 - protein, 219 aa, Homo sapiens
DIVMTQSPLSLPVTPGEPASISCRSSQSLLHSDGYNCLDWYLQKPGQSPQLLIYLGSTRASGFPDRFSGSGSGTDFTLKISRVEAEDVGVYYCMQALQTPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 404 - DNA, 657 bp, Homo sapiens; identical to SEQ ID NO: 390
  source 1..657: mol_type = unassigned DNA

SEQ ID NO: 405 - protein, 8 aa, Homo sapiens
GVTFDDYG

SEQ ID NO: 406 - protein, 8 aa, Homo sapiens
INWNGGDT

SEQ ID NO: 407 - protein, 17 aa, Homo sapiens
ARDFYGSGSYYHVPFDY

SEQ ID NO: 408 - protein, 124 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 409 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 410 - protein, 454 aa, Homo sapiens
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggartgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 412 - protein, 7 aa, Homo sapiens
QSVSRSY

SEQ ID NO: 413 - skipped

SEQ ID NO: 414 - protein, 9 aa, Homo sapiens
HQYDMSPFT

SEQ ID NO: 415 - protein, 108 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 416 - DNA, 324 bp, Homo sapiens
  source 1..324: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaa

SEQ ID NO: 417 - protein, 215 aa, Homo sapiens
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - DNA, 645 bp, Homo sapiens
  source 1..645: mol_type = unassigned DNA
gaaattgtgttgacgcagtctccagggaccctgtctttgtctccaggggaaagagccaccctctcctgcagggccagtcagagtgttagcagaagctacttagcctggtaccagcagaaacgtggccaggctcccaggctcctcatctatggtgcatccagcagggccactggcatcccagacaggttcagtggcgatgggtctgggacagacttcactctctccatcagcagactggagcctgaagattttgcagtgtattactgtcaccagtatgatatgtcaccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 419 - protein, 8 aa, Homo sapiens
GLTFDDYG

SEQ ID NO: 420 - protein, 8 aa, Homo sapiens
INWNGDNT

SEQ ID NO: 421 - protein, 17 aa, Homo sapiens
ARDYYGSGSYYNVPFDY

SEQ ID NO: 422 - protein, 124 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSS

SEQ ID NO: 423 - DNA, 372 bp, Homo sapiens
  source 1..372: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctca

SEQ ID NO: 424 - protein, 454 aa, Homo sapiens
EVQLVESGGGVVRPGGSLRLSCAASGLTFDDYGMSWVRQVPGKGLEWVSGINWNGDNTDYADSVKGRFTISRDNAKNSLYLQMNSLRAEDTALYYCARDYYGSGSYYNVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - DNA, 1368 bp, Homo sapiens
  source 1..1368: mol_type = unassigned DNA
gaggtgcagctggtggagtctgggggaggtgtggtacggcctggggggtccctgagactctcctgtgcagcctctggactcacctttgatgattatggcatgagctgggtccgccaagttccagggaaggggctggagtgggtctctggtattaattggaatggtgataacacagattatgcagactctgtgaagggccgattcaccatctccagagacaacgccaagaactccctgtatctgcaaatgaacagtctgagagccgaggacacggccttgtattactgtgcgagggattactatggttcggggagttattataacgttccttttgactactggggccagggaaccctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 426 - protein, 7 aa, Homo sapiens
QSVSSSY

SEQ ID NO: 427 - skipped

SEQ ID NO: 428 - protein, 8 aa, Homo sapiens
QQYGSSPF

SEQ ID NO: 429 - protein, 107 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIK

SEQ ID NO: 430 - DNA, 321 bp, Homo sapiens
  source 1..321: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaa

SEQ ID NO: 431 - DNA, 323 bp, Homo sapiens
  source 1..323: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaa

SEQ ID NO: 432 - protein, 214 aa, Homo sapiens
EIVLTQSPGTLSLSPGERATLSCRASQSVSSSYLAWYQQKPGQAPRLLIYGASSRATGIPDRFSGSGSGTDFTLTIRRLEPEDFAVYYCQQYGSSPFFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 433 - DNA, 642 bp, Homo sapiens
  source 1..642: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 434 - DNA, 644 bp, Homo sapiens
  source 1..644: mol_type = unassigned DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatatatggtgcatccagcagggccactggcatcccagacaggttcagtggcagtgggtctgggacagacttcactctcaccatcagaagactggagcctgaagattttgcagtgtattactgtcagcagtatggtagttcaccattcacttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 435 - protein, 8 aa, Homo sapiens
GYTFNSYG

SEQ ID NO: 436 - protein, 8 aa, Homo sapiens
ISVHNGNT

SEQ ID NO: 437 - protein, 17 aa, Homo sapiens
ARAGYDILTDFSDAFDI

SEQ ID NO: 438 - protein, 124 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSS

SEQ ID NO: 439 - DNA, 372 bp, Homo sapiens
  source 1..372: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttca

SEQ ID NO: 440 - protein, 454 aa, Homo sapiens
QVQLVQSGAEVKKPGASVKVSCKASGYTFNSYGIIWVRQAPGQGLEWMGWISVHNGNTNCAQKLQGRVTMTTDTSTSTAYMELRSLRTDDTAVYYCARAGYDILTDFSDAFDIWGHGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - DNA, 1368 bp, Homo sapiens
  source 1..1368: mol_type = unassigned DNA
caggttcagttggtgcagtctggagctgaggtgaagaagcctggggcctcagtgaaggtctcctgcaaggcttctggttacacctttaatagttatggtatcatctgggtgcgacaggcccctggacaagggcttgagtggatgggatggatcagcgttcacaatggtaacacaaactgtgcacagaagctccagggtagagtcaccatgaccacagacacatccacgagcacagcctacatggagctgaggagcctgagaactgacgacacggccgtgtattactgtgcgagagcgggttacgatattttgactgatttttccgatgcttttgatatctggggccacgggacaatggtcaccgtctcttcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 442 - protein, 6 aa, Homo sapiens
QNINNF

SEQ ID NO: 443 - skipped

SEQ ID NO: 444 - protein, 8 aa, Homo sapiens
QQSYGIPW

SEQ ID NO: 445 - protein, 106 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIK

SEQ ID NO: 446 - DNA, 318 bp, Homo sapiens
  source 1..318: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaa

SEQ ID NO: 447 - protein, 213 aa, Homo sapiens
DIQMTQSPSSLSASVGDRVTITCRASQNINNFLNWYQQKEGKGPKLLIYAASSLQRGIPSTFSGSGSGTDFTLTISSLQPEDFATYICQQSYGIPWVGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - DNA, 639 bp, Homo sapiens
  source 1..639: mol_type = unassigned DNA
gacatccagatgacccagtctccatcctccctgtctgcatctgtaggagacagagtcaccatcacttgccgggcaagtcagaacattaataactttttaaattggtatcagcagaaagaagggaaaggccctaagctcctgatctatgcagcatccagtttgcaaagagggataccatcaacgttcagtggcagtggatctgggacagacttcactctcaccatcagcagtctgcaacctgaagattttgcaacttacatctgtcaacagagctacggtatcccgtgggtcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 449 - protein, 8 aa, Homo sapiens
GFTFSDYF

SEQ ID NO: 450 - protein, 8 aa, Homo sapiens
ISSSGSTI

SEQ ID NO: 451 - protein, 21 aa, Homo sapiens
ARDHYDGSGIYPLYYYYGLDV

SEQ ID NO: 452 - protein, 128 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSS

SEQ ID NO: 453 - DNA, 384 bp, Homo sapiens
  source 1..384: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctca

SEQ ID NO: 454 - protein, 458 aa, Homo sapiens
QVQLVESGGGLVKPGGSLRLSCAASGFTFSDYFMSWIRQAPGKGLEWISYISSSGSTIYYADSVRGRFTISRDNAKYSLYLQMNSLRSEDTAVYYCARDHYDGSGIYPLYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - DNA, 1380 bp, Homo sapiens
  source 1..1380: mol_type = unassigned DNA
caggtgcagctggtggagtctgggggaggcttggtcaagcctggagggtccctgagactctcctgtgcagcctctggattcaccttcagtgactacttcatgagctggatccgccaggcgccagggaaggggctggagtggatttcatacattagttctagtggtagtaccatatactacgcagactctgtgaggggccgattcaccatctccagggacaacgccaagtactcactgtatctgcaaatgaacagcctgagatccgaggacacggccgtgtattactgtgcgagagatcactacgatggttcggggatttatcccctctactactattacggtttggacgtctggggccaggggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 456 - protein, 11 aa, Homo sapiens
QSLLHSNGYNY

SEQ ID NO: 457 - skipped

SEQ ID NO: 458 - protein, 9 aa, Homo sapiens
MQALQTPRS

SEQ ID NO: 459 - protein, 111 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIK

SEQ ID NO: 460 - DNA, 333 bp, Homo sapiens
  source 1..333: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaa

SEQ ID NO: 461 - protein, 218 aa, Homo sapiens
IVMTQSPLSLPVTPGEPASISCRSSQSLLHSNGYNYLDYYLQKPGQSPQLLIYLGSYRASGVPDRFSGSGSGTDFTLKISRVEAEDVGVYYCMQALQTPRSFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - DNA, 654 bp, Homo sapiens
  source 1..654: mol_type = unassigned DNA
attgtgatgactcagtctccactctccctacccgtcacccctggagagccggcctccatctcctgcaggtctagtcagagcctcctgcatagtaatggatacaactatttggattattacctgcagaagccagggcagtctccacagctcctgatctatttgggttcttatcgggcctccggggtccctgacaggttcagtggcagtggatcaggcacagattttacactgaaaatcagcagagtggaggctgaggatgttggggtttattactgcatgcaagctctacaaactcctcgcagttttggccaggggaccacgctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 463 - protein, 10 aa, Homo sapiens
GFSLSTTGVG

SEQ ID NO: 464 - protein, 7 aa, Homo sapiens
IYWDDDK

SEQ ID NO: 465 - protein, 16 aa, Homo sapiens
THGYGSASYYHYGMDV

SEQ ID NO: 466 - protein, 124 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 467 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 468 - protein, 454 aa, Homo sapiens
QITLKESGPTLVKPTQTLTLTCTFSGFSLSTTGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactactggagtgggtgtgggctggatccgtcagcccccaggaaaggccctggagtggcttgcagtcatttattgggatgatgataagcgctacagcccatctctgaagagcagactcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 470 - protein, 6 aa, Homo sapiens
QSVTNY

SEQ ID NO: 471 - skipped

SEQ ID NO: 472 - protein, 9 aa, Homo sapiens
QHRSNWPLT

SEQ ID NO: 473 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIK

SEQ ID NO: 474 - DNA, 322 bp, Homo sapiens
  source 1..322: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaac

SEQ ID NO: 475 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQHRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 476 - DNA, 643 bp, Homo sapiens
  source 1..643: mol_type = unassigned DNA
gaaattgtattgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaccgtagcaactggcctctcactttcggcggagggaccaaggtggagatcaaaccgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 477 - protein, 10 aa, Homo sapiens
GFSLSTSGVG

SEQ ID NO: 478 - protein, 7 aa, Homo sapiens
IYWDDDK

SEQ ID NO: 479 - protein, 16 aa, Homo sapiens
THGYGSASYYHYGMDV

SEQ ID NO: 480 - protein, 124 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSS

SEQ ID NO: 481 - DNA, 372 bp, Homo sapiens
  source 1..372: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctca

SEQ ID NO: 482 - protein, 454 aa, Homo sapiens
QITLKESGPTLVKPTQTLTLTCTFSGFSLSTSGVGVGWIRQPPGKALEWLAVIYWDDDKRYSPSLKSRLTITKDTSKNQVVLTMTNMDPVDTATYFCTHGYGSASYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - DNA, 1368 bp, Homo sapiens
  source 1..1368: mol_type = unassigned DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcaggctcaccatcaccaaggacacctccaaaaaccaggtggtccttacaatgaccaacatggaccctgtggacacagccacatatttctgtacacacggatatggttcggcgagttattaccactacggtatggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 484 - protein, 6 aa, Homo sapiens
QSVTNY

SEQ ID NO: 485 - skipped

SEQ ID NO: 486 - protein, 9 aa, Homo sapiens
QQRSNWPLT

SEQ ID NO: 487 - protein, 107 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIK

SEQ ID NO: 488 - DNA, 321 bp, Homo sapiens
  source 1..321: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 489 - protein, 214 aa, Homo sapiens
EIVLTQSPATLSLSPGERATLSCRASQSVTNYLAWHQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - DNA, 642 bp, Homo sapiens
  source 1..642: mol_type = unassigned DNA
gaaattgtgttgacacagtctccagccaccctgtctttgtctccaggggaaagagccaccctctcctgcagggccagtcagagtgttaccaactacttagcctggcaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 491 - protein, 8 aa, Homo sapiens
GFTFSDYY

SEQ ID NO: 492 - protein, 8 aa, Homo sapiens
ISSSGSTI

SEQ ID NO: 493 - protein, 20 aa, Homo sapiens
ARDFYDILTDSPYFYYGVDV

SEQ ID NO: 494 - protein, 127 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSS

SEQ ID NO: 495 - DNA, 381 bp, Homo sapiens
  source 1..381: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctca

SEQ ID NO: 496 - protein, 457 aa, Homo sapiens
QVQLVESGGGLVKPGGSLRLSCAASGFTFSDYYMSWIRQAPGKGLEWVSYISSSGSTIYYADSVKGRFTISRDNAKNSLYLQINSLRAEDTAVYYCARDFYDILTDSPYFYYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 497 - DNA, 1377 bp, Homo sapiens
  source 1..1377: mol_type = unassigned DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaattaacagcctgagagccgaggacacggccgtgtattactgtgcgagagatttttacgatattttgactgatagtccgtacttctactacggtgtggacgtctggggccaagggaccacggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 498 - protein, 11 aa, Homo sapiens
QSLLHSNGYNY

SEQ ID NO: 499 - skipped

SEQ ID NO: 500 - protein, 9 aa, Homo sapiens
MQALQTPRT

SEQ ID NO: 501 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 502 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 503 - protein, 219 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 505 - protein, 239 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTIEGRDYKDDDDKHHHHHH

SEQ ID NO: 506 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 507 - protein, 121 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKF

SEQ ID NO: 508 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 509 - protein, 33 aa, Homo sapiens
KYSSSVHDPNGEYMFMRAVNTAKKSRLTDVTLM

SEQ ID NO: 510 - protein, 128 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 511 - protein, 121 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLK

SEQ ID NO: 512 - protein, 147 aa, Mus musculus
MGWSCIILFLVATATGVHSEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKIVVQVTE

SEQ ID NO: 513 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 514 - protein, 120 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLK

SEQ ID NO: 515 - protein, 240 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWS

SEQ ID NO: 516 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 517 - protein, 113 aa, Homo sapiens
LQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 518 - protein, 113 aa, Homo sapiens
LQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 519 - DNA, 336 bp, Homo sapiens
  source 1..336: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaa

SEQ ID NO: 520 - DNA, 657 bp, Homo sapiens
  source 1..657: mol_type = unassigned DNA
gatattgtgatgactcagtctccactctccctgcccgtcacccctggagagccggcctccatctcctgcaggtctagtcagagcctcctgcatagtgatggatacaactatttggattggtacctgcagaagccagggcagtctccacagctcctgatctatttgggttctactcgggcctccgggttccctgacaggttcagtggcagtggatcaggcacagattttacactgaaaatcagcagagtggaggctgaggatgttggggtttattactgcatgcaagctctacaaactccgctcagttttggccaggggaccaagctggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 521 - DNA, 372 bp, Homo sapiens
  source 1..372: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctca

SEQ ID NO: 522 - DNA, 1368 bp, Homo sapiens
  source 1..1368: mol_type = unassigned DNA
gaggtgcagctggtggagtctgggggaggtgtggtacggcctggggggtccctgagactctcctgtgtagcctctggagtcacctttgatgattatggcatgagctgggtccgccaagctccagggaaggggctggagtgggtctctggtattaattggaatggtggcgacacagattattcagactctgtgaagggccgattcaccatctccagagacaacgccaagaactccctgtatctacaaatgaatagtctgagagccgaggacacggccttgtattactgtgcgagggatttctatggttcggggagttattatcacgttccttttgactactggggccagggaatcctggtcaccgtctcctcagccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagcagggacgagctgaccaagaaccaggtgtccctgacctgtctcgtgaaaggcttctacccctccgatatcgccgtggaatgggagtccaacggccagcctgagaacaactacaagaccaccccccctgtgctggactccgacggctcattcttcctgtacagcaagctgacagtggacaagtcccggtggcagcagggcaacgtgttctcctgctccgtgatgcacgaggccctgcacaaccactacacccagaagtccctgtccctgagccccggcaagtgatga

SEQ ID NO: 523 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 524 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 526 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 527 - DNA, 978 bp, Homo sapiens
  source 1..978: mol_type = unassigned DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 528 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 529 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 530 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 531 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 532 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 533 - DNA, 978 bp, Homo sapiens
  source 1..978: mol_type = unassigned DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 534 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 535 - DNA, 318 bp, Homo sapiens
  source 1..318: mol_type = unassigned DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 536 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 537 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 538 - protein, 106 aa, Homo sapiens; identical to SEQ ID NO: 225

SEQ ID NO: 539 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 215 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 541 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 542 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 217 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 544 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 545 - protein, 663 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 546 - protein, 214 aa, Homo sapiens
EVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 547 - protein, 220 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 548 - protein, 667 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 549 - protein, 216 aa, Homo sapiens
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 550 - protein, 214 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 551 - protein, 659 aa, Homo sapiens
DIQMTQSPASLSASLGETVTIQCRASEDIYSGLAWFQQKPGKSPQLLIYGASSLQDGVPSRFSGSGSGTQYSLKISSMQTEDEGVYFCQQGLKYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 552 - protein, 215 aa, Homo sapiens; identical to SEQ ID NO: 540

SEQ ID NO: 553 - protein, 220 aa, Homo sapiens; identical to SEQ ID NO: 541

SEQ ID NO: 554 - protein, 663 aa, Homo sapiens
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGRSLKLSCAASGFTFSDFYMAWVRQAPKKGLEWVASISYEGSSTYYGDSVMGRFTISRDNAKSTLYLQMNSLRSEDTATYYCARQREANWEDWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 555 - protein, 217 aa, Homo sapiens; identical to SEQ ID NO: 543

SEQ ID NO: 556 - protein, 214 aa, Homo sapiens; identical to SEQ ID NO: 544

SEQ ID NO: 557 - protein, 659 aa, Homo sapiens; identical to SEQ ID NO: 551

SEQ ID NO: 558 - protein, 215 aa, Homo sapiens; identical to SEQ ID NO: 540

SEQ ID NO: 559 - protein, 220 aa, Homo sapiens; identical to SEQ ID NO: 541

SEQ ID NO: 560 - protein, 663 aa, Homo sapiens; identical to SEQ ID NO: 554

SEQ ID NO: 561 - protein, 217 aa, Homo sapiens; identical to SEQ ID NO: 543

SEQ ID NO: 562 - protein, 214 aa, Homo sapiens; identical to SEQ ID NO: 544

SEQ ID NO: 563 - protein, 11 aa, synthetic construct
  REGION 1..11: note = Description of Artificial Sequence: Synthetic peptide
  VAR_SEQ 1: note = Any amino acid
  VAR_SEQ 5: note = Any amino acid
  VAR_SEQ 8: note = Any amino acid
  VAR_SEQ 9: note = Any amino acid or absent
XGSGXYGXXFD

SEQ ID NO: 564 - protein, 11 aa, synthetic construct
  REGION 1..11: note = Description of Artificial Sequence: Synthetic peptide
  VAR_SEQ 3: note = Any amino acid
  VAR_SEQ 5..6: note = Any amino acid
  VAR_SEQ 9..10: note = Any amino acid
ARXRXXSDXXD

SEQ ID NO: 565 - protein, 8 aa, synthetic construct
  REGION 1..8: note = Description of Artificial Sequence: Synthetic peptide
  MOD_RES 1: note = Any amino acid
  VAR_SEQ 1: note = Any amino acid
XRDGSGSY